(19) United States
(12) Patent Application Publication
(10) Pub. No.: US 2025/0205344 A1
(43) Pub. Date: Jun. 26, 2025

(54) 2,4-DIOXOTETRAHYDROPYRIMIDINYL DERATIVES AS DEGRONS IN PROTACS

(71) Applicant: GLAXOSMITHKLINE INTELLECTUAL PROPERTY DEVELOPMENT LIMITED, Stevenage (GB)

(72) Inventors: Paul BAMBOROUGH, Stevenage (GB); John David HARLING, Stevenage (GB); Afjal Hussain MIAH, Stevenage (GB); Alan John NADIN, Stevenage (GB); Craig Michael ROBERTSON, Stevenage (GB); Rishi Rajnikant SHAH, Stevenage (GB); Ian Edward David SMITH, Stevenage (GB); Christopher Patrick TINWORTH, Stevenage (GB); Christopher Roland WELLAWAY, Stevenage (GB)

(21) Appl. No.: 18/849,815

(22) PCT Filed: Mar. 22, 2023
(86) PCT No.: PCT/EP2023/057338

§ 371 Date: Sep. 23, 2024

(30) Foreign Application Priority Data
Mar. 24, 2022 (GB) 2204142.0
Sep. 22, 2022 (GB) 2213816.8
Feb. 1, 2023 (GB) 2301448.3
Feb. 8, 2023 (GB) 2301757.7

(51) Int. Cl. A61K 47/55; A61K 47/54; C07D 401/14; C07D 403/04; C07D 403/14; C07D 471/04; C07D 471/10; C07D 487/04

(52) U.S. Cl. CPC A61K 47/55; A61K 47/545; C07D 401/14; C07D 403/04; C07D 403/14; C07D 471/04; C07D 471/10; C07D 487/04

(57) ABSTRACT

The present invention provides a series of 2,4-dioxotetrahydropyrimidinyl derivatives that bind cereblon, and their application as degrons in PROTACs. Androgen receptor PROTACs containing the 2,4-dioxotetrahydropyrimidinyl containing degrons and medical uses of these PROTACS are also disclosed.

### FIELD OF THE INVENTION

The present invention provides a series of 2,4-dioxotetrahydropyrimidinyl derivatives that bind cereblon, and their application as degrons in PROTACs. Androgen receptor PROTACs containing the 2,4-dioxotetrahydropyrimidinyl containing degrons and medical uses of these PROTACS are also disclosed.

### BACKGROUND TO THE INVENTION

The Ubiquitin Proteosome Pathway System (UPS) is a pathway for degrading regulatory proteins as well as misfolded or abnormal proteins. It achieves this by post translational modification of substrate proteins by the covalent attachment of ubiquitin. The covalent attachment of ubiquitin to specific protein substrates is achieved through the action of E3 ubiquitin ligases. Over 500 E3 ubiquitin ligases are known. One such E3 ubiquitin ligase is Cereblon. Cereblon (CRBN) forms a complex with damaged DNA binding protein 1 (DDB1), Cullin-4A (CUL4A), and regulator of cullins 1 (ROC1). This complex recognises naturally occurring protein substrates and catalyses the addition of ubiquitin, targeting the substrates for destruction.

The PROTAC (PROteolysis TArgeting Chimeric) approach hijacks the UPS to degrade proteins that would not normally be substrates. PROTAC compounds are compounds that are typically comprised of three parts, a portion dedicated to binding to the target protein (target binding moiety), a portion binding to an E3 ligase capable of recruiting the complex capable of ubiquitinating the target protein (degron), and a linker connecting the two portions. Upon binding to a target, the PROTAC generates a ternary complex of all of the components needed to ubiquitinate the target, leading to ubiquitination and subsequently degradation of the target.

Cereblon is a molecular target of immunomodulatory agents such as thalidomide, lenalidomide, and pomalidomide. These agents have been widely used as E3 ligase binding moieties in PROTAC compounds. WO2022069520 discloses compounds that are said to be cereblon binding moieties.

The PROTAC approach is a principle capable of general application and it is reasonable to expect that a PROTAC capable of degrading a particular target can be found using a validated target binding moiety and linkers known in the art. Despite this, it has become clear that not all PROTACs have equivalent activity in vitro or in vivo. There are several reasons for this including differential absorption through the intestinal tract, differential absorption into the cells, differential pharmacokinetics and the differential ability of the PROTAC molecule to form a functional ternary complex (a pre-requisite to ubiquitination and degradation of the target). Additionally, for some reasons that are not entirely clear, differential selectivity of PROTACs for their target has been observed. Accordingly, it is clear that, whilst the PROTAC approach is one of general application, it is not correct to view them as modular molecules, and that optimisation is possible by modification of each PROTAC component.

Androgens normally exert their biological effects via binding to the Androgen Receptor (AR). In the absence of androgens, AR is bound by Heat Shock Protein 90 (Hsp90) in the cytosol in such as way as to mask the Nuclear Localisation Signal (NLS). When an androgen binds AR. This induces a conformational change leading to the release of Hsp90, exposing the NLS. The AR then translocates into the nucleus where it acts as a transcription factor (Endocrin Rev. 1987, 8(1): 1-28; Mol Endocrinol. 2002, 16(10), 2181-7).

Androgens have long been known to be associated with prostate carcinogenesis. The evidence for this comes from several sources. First, androgens induce prostate cancer in rodent animal models (Noble, Cancer Res., 37, 1929-1933 (1977)): Second, men receiving androgens in the form of anabolic steroids have a higher incidence of prostate cancer (Roberts and Essenhigh, Lancet, 2, 742 (1986) and prostate cancer does not develop following castration (Wilson and Roehrborn, J Clin Endrocrin Metab, 84, 4324-4331, 1999). Finally, and most convincingly, the only effective treatment available for advanced prostate cancer is withdrawal of androgens and is referred to as androgen ablation therapy (ABT) or androgen depravation therapy (ADT) or chemical castration. However, most patients develop resistance and the disease progresses (Huber et al., 1987, Scan J Urol 104, 33-39).

Castration resistant prostate cancer cells have undergone changes to enable them to survive under castration levels of androgen. These mechanisms include AR overexpression, changes to androgen biosynthesis, the expression of constitutively active AR splice variants, changes to androgen cofactors and the expression of mutated versions of the AR. For example, gain of function mutations in the ligand binding domain of the AR such as L702H, W742C, W742L, H875Y and T878A can change ligand binding affinity, which results in increased sensitivity to steroid ligands or the conversion of anti-androgens to agonists. The T878A mutation is associated with resistance to abiraterone acetate and hydroxyflutamide. The L702H mutation is associated with receptor promiscuity, that is increased AR sensitivity to glucocorticoids.

AR is therefore a critical driver of tumorigenesis in prostate cancer, including castration resistant prostate cancer and its elimination should lead to therapeutically beneficial response.

In addition to their role in prostate cancer, androgens also play a role in other diseases. One example is ovarian cancer where elevated levels of androgens are associated with an increased risk of developing ovarian cancer (Helzlsouer et al., JAMA 274, 1926-1930 (1995), Edmondson et al., Br J Cancer 86, 879-885 (2002)). Indeed, the androgen receptor is detected in the majority of ovarian cancers (Risch, J. Natl. Cancer Inst., 90, 1774-1786, 1998, .Rao and Slotman, Endocr Rev., 12, 14-26, 1991, Clinton and Hua, Crit Rev Oncol., Hematol., 25, 1-9, 1997)).

### SUMMARY OF THE INVENTION

In a first aspect, the invention provides a compound of formula (I), a tautomer of a compound of formula (I), or a salt thereof:

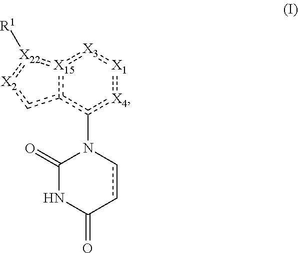

wherein:
X₁ is N or C—R² wherein R² is selected from the group consisting of hydrogen, halogen, C₁₋₄alkyl, C₁₋₄haloalkyl, C₁₋₄alkoxy, —CONR⁵R⁶ and —(CONR³R⁴)m(L)p(TBM)q;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
n is 0 or 1;
R⁵ and R⁶ are independently selected from hydrogen or C₁₋₄alkyl;
R¹ is C₁₋₄alkyl, C₁₋₄haloalkyl, C₁₋₄alkoxy or a group of formula (II), wherein * represents the position of attachment to the compound of formula (I):

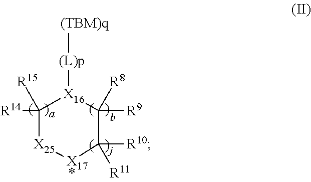

a and b are independently 0 or 1;
X₁₆ is N or CH;
X₁₇ is CR³⁴R³⁵ wherein either R³⁴ and R³⁵ together with the carbon atom to which they are attached, join together to form a cyclobutyl ring, or wherein R³⁴ is —(CHR³⁶)r-, or a bond to the compound of formula (I) and R³⁵ is hydrogen or halogen;
X₂₅ is CR¹²R¹³ or O;
r is 0, 1 or 2;
a is 0, 1 or 2 and b and j are independently 0 or 1 with the proviso that b and j cannot both be 0;
R³⁶ is hydrogen or methyl;
R⁸, R⁹, R¹⁰, R¹¹, R¹², R¹³, R¹⁴ and R¹⁵ are independently selected from hydrogen or halogen;
L is a chemical linker;
TBM is a target binding moiety;
m, p and q are independently 0 and 1;
wherein when X₁₅ is N, then X₂₂ is C; and
wherein when X₁ is —(CONR³R⁴)m(L)p(TBM)q, then R¹ is C₁₋₄alkyl, C₁₋₄haloalkyl or C₁₋₄alkoxy.

For the avoidance of doubt, the double dotted lines in the 5,6-bicyclic ring structure describe an aromatic structure.

The single dotted line paired with a single solid line in the 6-membered monocyclic ring may either be a single bond or a double bond.

In one embodiment, the invention provides a compound of formula (I), a tautomer of a compound of formula (I), or a salt thereof:

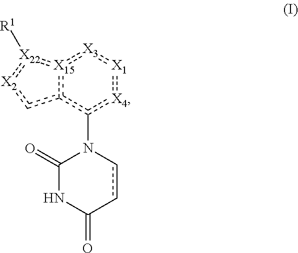

wherein:
X₁ is N or C—R² wherein R² is selected from the group consisting of hydrogen, halogen, C₁₋₄alkyl, C₁₋₄haloalkyl, C₁₋₄alkoxy, —CONR⁵R⁶ and —CONR³R⁴(L)p(TBM)q;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
n is 0 or 1;
R⁵ and R⁶ are independently selected from H or C₁₋₄alkyl;
R¹ is C₁₋₄alkyl, C₁₋₄haloalkyl, C₁₋₄alkoxy or a group of formula (II), wherein * represents the position of attachment to the compound of formula (I):

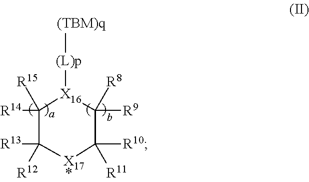

a and b are independently 0 or 1;
X₁₆ is N or CH;
X₁₇ is CR³⁴R³⁵ wherein either R³⁴ and R³⁵ together with the carbon atom to which they are attached, join together to form a cyclobutyl ring, or wherein R³⁴ is —(CHR³⁶)—, or a bond to the compound of formula (I) and R³⁵ is H or a halogen;
R³⁶ is hydrogen or methyl;
R⁸, R⁹, R¹⁰, R¹¹, R¹², R¹³, R¹⁴ and R¹⁵ are independently selected from H or halogen;
L is a chemical linker;
TBM is a target binding moiety;
p and q are independently 0 and 1;
wherein when X₁₅ is N, X₂₂ is C; and
wherein when X₁ is —CONR³R⁴(L)p(TBM)q, then R¹ is C₁₋₄alkyl, C₁₋₄haloalkyl or C₁₋₄alkoxy.

Compounds of formula (I), tautomers or salts thereof wherein in which p and q are each 0 are degrons capable of binding cereblon. When incorporated into PROTACs, a degron is capable of recruiting the a complex capable of ubiquitinating the target protein.

In particular embodiments, the invention provides degrons of formulae (Iaa) and (Ibb) and (Icc), tautomers or salts thereof, wherein X₁, X₂, X₃, X₄, X₁₅, X₁₆, X₂₂, X₂₅, R¹, R³, R⁴, R⁸, R⁹, R¹⁰, R¹¹, R¹², R¹³, R¹⁴, R¹⁵, R³⁵, R³⁶, a, b m, j, k, l and r are defined as for formula (I), X₁₈ is CR³⁵. 1.

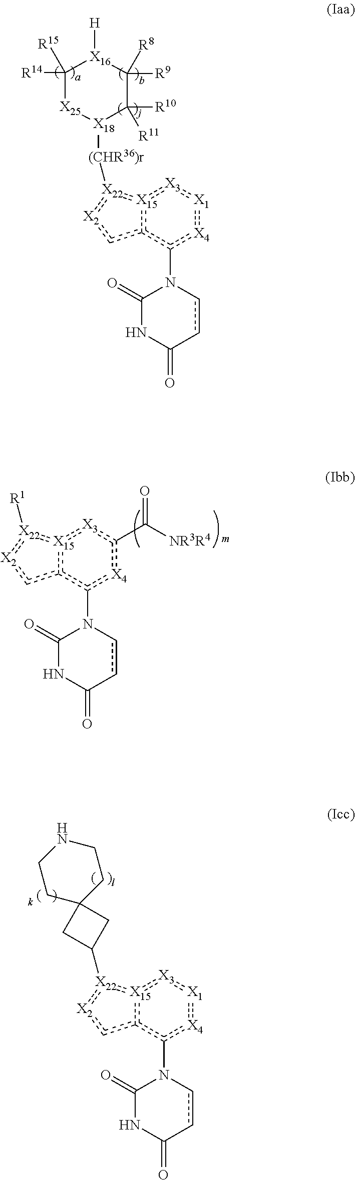

In other embodiments, the invention provides PROTACs comprising the degron of formula (I), (Iaa), (Ibb) or (Icc) or a tautomer thereof. Compounds of formula (I), tautomers or salts thereof in which p is 1 are “PROTACs”.

In particular embodiments, the invention provides PROTACs in which the target binding moiety is an androgen receptor binding moiety. Pharmaceutical compositions and medical uses of androgen receptor PROTACs are also provided.

### DETAILED DESCRIPTION OF THE INVENTION

### Definitions

The term “alkyl” refers to a monovalent, saturated hydrocarbon radical, straight or branched, having the specified number of carbon atoms. For example, the term “C₁₋₄ alkyl” refers to an alkyl group having 1 to 4 carbon atoms. Exemplary groups include, but are not limited to, methyl, ethyl, propyl (n-propyl and isopropyl) and butyl (n-butyl, sec-butyl, isobutyl and tert-butyl).

The term “alkoxy” refers to an —O-alkyl group, i.e. an alkyl group which is attached through an oxygen linking atom, wherein “alkyl” is defined above. For example, the term “C₁₋₄ alkoxy” refers to an alkoxy group having 1 to 4 carbon atoms. Exemplary groups include, but are not limited to, methoxy, ethoxy, n-propoxy, isopropoxy, n-butoxy, s-butoxy, isobutoxy, and t-butoxy.

The term “cycloalkyl” refers to a non-aromatic, saturated, monocyclic, hydrocarbon ring containing the specified number of carbon atoms. For example, C₃₋₆ cycloalkyl contains 3 to 6 carbon atoms as member atoms in the ring. Examples of C₃₋₆ cycloalkyl include, but are not limited to, cyclobutyl, cyclopentyl and cyclohexyl. The term “cycloalkylene” refers specifically to a bivalent cycloalkyl ring.

The terms “halogen” and “halo” represent chloro, fluoro, bromo, or iodo substituents.

The term “haloalkyl” “is intended to mean a radical having one or more halogen atoms, which may be the same or different, at one or more carbon atoms of an alkyl group, where “alkyl” is defined above. Exemplary groups include, but are not limited to, —CF₃ (trifluoromethyl), —CCl₃ (trichloromethyl), 1,1-difluoroethyl, 2,2,2-trifluoroethyl, and hexafluoroisopropyl.

The term “heteroaryl” refers to a group or moiety comprising an aromatic monovalent monocyclic or bicyclic radical, containing 5 to 10 ring atoms, including at least one heteroatom independently selected from nitrogen, oxygen and sulfur. This term also encompasses bicyclic heterocyclic-aryl compounds containing an aryl ring moiety fused to a heterocycloalkyl ring moiety, containing 5 to 10 ring atoms, including at least one heteroatom independently selected from nitrogen, oxygen and sulfur. The number of ring atoms may be specified. For example, a “6-membered heteroaryl,” is a heteroaryl monocyclic ring as defined above consisting of six ring atoms. The presence of particular heteroatoms may also be specified. For example, a “6 membered nitrogen containing heteroaryl” is a 6-membered heteroaryl as defined above containing at least one nitrogen atom. Examples of 6-membered nitrogen containing heteroaryl groups include pyridinyl, pyridazinyl, pyrazinyl, and pyrimidinyl. The term “heteroarylene” refers specifically to a bivalent heteroaryl group.

The term “heterocyclic ring” refers to a saturated or unsaturated 3 to 10 membered monocyclic or bicyclic ring, which must contain at least one heteroatom, which is selected from nitrogen, oxygen, and sulfur. Heterocyclic rings may contain one or more C(O), S(O) or SO₂ groups. Bicyclic heterocyclic rings may be fused, bridged or spiro bicyclic groups. However, heterocyclic rings are not aromatic. Heterocyclic rings containing more than one heteroatom may contain different heteroatoms. The number of ring atoms may be specified. For example, a “6-membered heterocyclylic ring,” is a heterocyclylic ring as defined above consisting of six ring atoms. The presence of particular heteroatoms may also be specified. For example, a “6 membered nitrogen containing heterocyclic ring” is a 6-membered heterocyclylic ring as defined above containing at least one nitrogen atom. Exemplary 6 membered nitrogen containing heterocyclic rings include, but are not limited to, piperidinyl, piperazinyl, morpholinyl, morpholinyl-3-one, piperidyl-2-one and pyrimidinyl-2,4(1H,3H)-dione. The term heterocyclene refers specifically to a bivalent heterocyclic ring.

The term “target binding moiety” refers to a chemical moiety capable of binding to a target protein, in particular a protein of therapeutic importance. The nature of the target is not limited, other than it must be an intracellular protein or a protein comprising an intracellular domain.

### STATEMENT OF THE INVENTION

In a first aspect, the invention provides a compound of formula (I), a tautomer of a compound of formula (I), or a salt thereof:

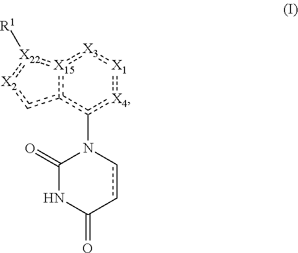

wherein:
X₁ is N or C—R² wherein R² is selected from the group consisting of hydrogen, halogen, C₁₋₄alkyl, C₁₋₄haloalkyl, C₁₋₄alkoxy, —CONR⁵R⁶ and —(CONR³R⁴)m(L)p(TBM)q;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
n is 0 or 1;
R⁵ and R⁶ are independently selected from hydrogen or C₁₋₄alkyl;
R¹ is C₁₋₄alkyl, C₁₋₄haloalkyl, C₁₋₄alkoxy or a group of formula (II), wherein * represents the position of attachment to the compound of formula (I):

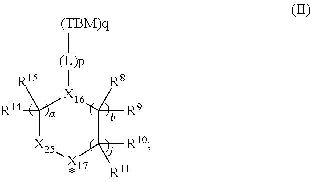

a and b are independently 0 or 1;
X₁₆ is N or CH;
X₁₇ is CR³⁴R³⁵ wherein either R³⁴ and R³⁵ together with the carbon atom to which they are attached, join together to form a cyclobutyl ring, or wherein R³⁴ is —(CHR³⁶)r-, or a bond to the compound of formula (I) and R³⁵ is hydrogen or halogen;
X₂₅ is CR¹²R¹³ or O;
r is 0, 1 or 2;
a is 0, 1 or 2 and b and j are independently 0 or 1 with the proviso that b and j cannot both be 0;
R³⁶ is hydrogen or methyl;
R⁸, R⁹, R¹⁰, R¹¹, R¹², R¹³, R¹⁴ and R¹⁵ are independently selected from hydrogen or halogen;
L is a chemical linker;
TBM is a target binding moiety;
m, p and q are independently 0 and 1;
wherein when X₁₅ is N, then X₂₂ is C; and
wherein when X₁ is —(CONR³R⁴)m(L)p(TBM)q, then R¹ is C₁₋₄alkyl, C₁₋₄haloalkyl or C₁₋₄alkoxy.

A compound of formula (I), a tautomer or a salt thereof may be degron or a PROTAC depending upon the values of p and q. PROTACs are compounds or salts containing a degron, a target binding moiety and optionally a linker. The “components” of a PROTAC will be described further below.

In one embodiment, q is 1 and p is 0 or 1.

In one embodiment, m is 1.

In one embodiment, X₁ is N or C—R² wherein R² is selected from the group consisting of hydrogen, halogen, C₁₋₄alkyl, C₁₋₄haloalkyl, and —CONR⁵R⁶.

### Degron

When p and q are both 0, the compound of formula (I) or tautomer thereof is a degron, capable of binding the E3 ligase cereblon.

The invention provides a degron which is a compound of formula (Iaa), a tautomer of a compound of formula (Iaa), or a salt thereof:

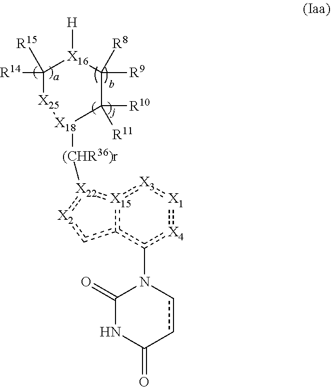

wherein:
X₁ is N or C—R² wherein R² is selected from the group consisting of hydrogen, halogen, C₁₋₄alkyl, C₁₋₄haloalkyl, C₁₋₄alkoxy and —CONR⁵R⁶;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
r is 0, 1 or 2;
a is 0, 1 or 2 and b and j are independently 0 or 1 with the proviso that b and j cannot both be 0;
X₁₆ is N or CH;
X₁₈ is CR³⁵, wherein R³⁵ is hydrogen or halogen;
X₂₅ is CR¹²R¹³ or O;
R⁵ and R⁶ are independently selected from hydrogen or C₁₋₄alkyl;
R⁸, R⁹, R¹⁰, R¹¹, R¹², R¹³, R¹⁴, R¹⁵ and R³⁵ are independently selected from hydrogen or halogen;
R³⁶ is hydrogen or methyl; and
wherein when X₁₅ is N, then X₂₂ is C.

In a more particular embodiment, the invention provides a degron which is a compound of formula (Iaaa), a tautomer of a compound of formula (Iaaa), or a salt thereof:

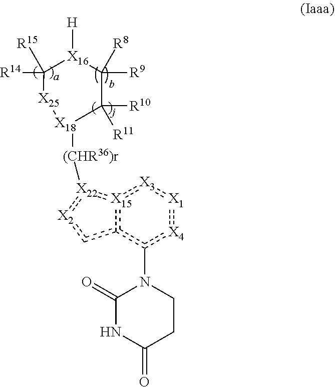

wherein:
X₁ is N or C—R² wherein R² is selected from the group consisting of hydrogen, halogen, C₁₋₄alkyl, C₁₋₄haloalkyl, C₁₋₄alkoxy and —CONR⁵R⁶;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
r is 0, 1 or 2;
a is 0, 1 or 2 and b and j are independently 0 or 1 with the proviso that b and j cannot both be 0;
X₁₆ is N or CH;
X₁₈ is CR³⁵, wherein R³⁵ is hydrogen or halogen;
X₂₅ is CR¹²R¹³ or O;
R⁵ and R⁶ are independently selected from hydrogen or C₁₋₄alkyl;
R⁸, R⁹, R¹⁰, R¹¹, R¹², R¹³, R¹⁴, R¹⁵ and R³⁵ are independently selected from H or halo;
R³⁶ is hydrogen or methyl; and
wherein when X₁₅ is N, then X₂₂ is C.

In one embodiment of the compound of formula (Iaa) or (Iaaa), tautomer or salt thereof, X₁ is N or C—R² wherein R² is selected from the group consisting of hydrogen, halogen, C₁₋₄alkyl, C₁₋₄haloalkyl, and —CONR⁵R⁶.

In one embodiment of the compound of formula (Iaa) or (Iaaa), tautomer or salt thereof, R² is selected from hydrogen and halo.

In one embodiment of the compound of formula (Iaa) or (Iaaa), tautomer or salt thereof, X₂₂ is N. In certain embodiments in which X₂₂ is N, one or more of X₁, X₂, X₃, X₄, are additionally N.

In one embodiment of the compound of formula (Iaa) or (Iaaa), tautomer or salt thereof, X₁₅ is C.

In one embodiment of the compound of formula (Iaa) or (Iaaa), tautomer or salt thereof, X₁ is N or CH.

In another embodiment of formula (Iaa) or (Iaaa), tautomer or salt thereof, X₁ is C—R² wherein R² is selected from the group consisting of CF₃, F or Cl. In a more particular embodiment, X₁ is C—R² wherein R² is selected from the group consisting of F or Cl.

In one embodiment of the compound of formula (Iaa) or (Iaaa), tautomer or salt thereof, X₄ is C—R⁷. In a more particular embodiment, X₄ is C—R⁷ wherein R⁷ is F, Cl or CF₃. In one embodiment, X₄ is C—R⁷ wherein R⁷ is F. In another embodiment, X₄ is CH. In an alternative embodiment, X₄ is N.

In one embodiment of the compound of formula (Iaa) or (Iaaa), tautomer or salt thereof, where X₂₂ is N, X₁ is CR² wherein R² is H, halogen or C₁₋₄haloalkyl, X₂ and X₃, are both CH, X₄ is CR⁷ wherein R⁷ is hydrogen or halogen, and X₁₅ is C. In a more particular embodiment, X₂₂ is N, X₁, X₂, X₃, X₄ are each CH and X₁₅ is C.

In certain embodiments in which X₂₂ is N, X₁, X₂, X₃, X₄ are each CH and X₁₅ is C, either:
a, b and j are not each 1, or
r is 1.

In one embodiment in which X₂₂ is N, X₁, X₂, X₃, X₄ are each CH and X₁₅ is C, the compound of formula (Iaa) or (Iaaa) is not 1-(1-(piperidin-4-yl)-1H-indol-4-yl)dihydropyrimidine-2,4-(1H, 3H)-dione.

In another embodiment where X₂₂ and X₂ are N, X₁, is CR² wherein R² is H, halogen or C₁₋₄haloalkyl, X₃ is CH, X₄ is CR⁷ wherein R⁷ is hydrogen or halogen, and X₁₅ is C. In another embodiment where X₂₂ is N, X₁, X₃ and X₄ are each CH, X₁₅ is C and X₂ is N. In a further embodiment where X₂₂ is N, X₁ is N and X₂, X₃ and X₄ are each CH and X₁₅ is C. In an alternative embodiment where X₂₂ is N, X₁ and X₂ are each N and X₃, and X₄ are each CH and X₁₅ is C. In an alternative embodiment where X₂₂ is N, X₂ and X₄ are each N and X₁, and X₃ are each CH and X₁₅ is C. In one embodiment where X₂₂ is N, X₃ and X₄ are each N, is CR² wherein R² is H, halogen, X₂ is CH and X₁₅ is C. In an alternative embodiment where X₂₂ is N, X₃ and X₄ are each N, X₁ and X₂ are each CH and X₁₅ is C. In an alternative embodiment where X₂₂ is N, X₂ and X₃ are each N, X₁ and X₄ are each CH and X₁₅ is C. In an alternative embodiment where X₂₂ is N, X₄ is N, X₁, X₂ and X₃ are each CH and X₁₅ is C. In an alternative embodiment where X₂₂ is N, X₃ is N, X₁, X₂ and X₄ are each CH and X₁₅ is C.

In an alternative embodiment of the compound of formula (Iaa) or (Iaaa), tautomer or salt thereof, where X₂ and X₁₅ are N, X₁, X₃, X₄ and are each CH and X₂₂ is C.

In one embodiment of the compound of formula (Iaa) or (Iaaa), tautomer or salt thereof, R³⁵ is CH.

In one embodiment of the compound of formula (Iaa) or (Iaaa), tautomer or salt thereof, r is 0 or 1. In one embodiment of the compound of formula (Iaa), tautomer a salt thereof, r is 0. In an alternative embodiment, r is 1 and R³⁶ is H. In an alternative embodiment, r is 1 and R³⁶ is methyl.

In an alternative embodiment of the compound of formula (Iaa) or (Iaaa), tautomer or salt thereof, r is 2 and and each R³⁶ is H.

In one embodiment of the compound of formula (Iaa) or (Iaaa), tautomer or salt thereof, X₁₆ is N.

In one embodiment of the compound of formula (Iaa) or (Iaaa), tautomer or salt thereof, X₂₅ is CR¹²R¹³.

In one embodiment of the compound of formula (Iaa) or (Iaaa), tautomer or salt thereof, j is 1 and a and b are independently 0 or 1. In a more particular embodiment, X₁₆ is N, X₂₅ is CR¹²R¹³, j is 1 and a and b are independently 0 or 1.

In one embodiment of the compound of formula (Iaa) or (Iaaa), tautomer or salt thereof, a, b and j are each 1, X₁₆ is CH or N and X₂₅ is CR¹²R¹³. In a more particular embodiment, a, b and j are both 1, X₁₆ is N and X₂₅ is CR¹²R¹³. In an alternative embodiment, a, b and j are both 1, X₁₆ is CH and X₂₅ is CR¹²R¹³.

In one embodiment of the compound of formula (Iaa) or (Iaaa), tautomer or salt thereof, a is 0 and b and j are each 1, X₁₆ is N and X₂₅ is CR¹²R¹³.

In one embodiment of the compound of formula (Iaa) or (Iaaa), tautomer or salt thereof, a and b are both 0, j is 1, X₁₆ is CH and X₂₅ is CR¹²R¹³.

In an alternative embodiment of the compound of formula (Iaa) or (Iaaa), tautomer or salt thereof, a and b are both 0, j is 1, X₁₆ is N and X₂₅ is CR¹²R¹³.

In one embodiment of the compound of formula (Iaa) or (Iaaa), tautomer or salt thereof, a is 2, b is 0, j is 1, X₁₆ is N and X₂₅ is CR¹²R¹³.

In one embodiment of the compound of formula (Iaa) or (Iaaa), tautomer or salt thereof, a is 2, b is 0, j is 1, X₁₆ is N and X₂₅ is O.

In one embodiment of the compound of formula (Iaa) or (Iaaa), tautomer or salt thereof, R⁸, R⁹, R¹⁰, R¹¹, R¹², R¹³, R¹⁴, R¹⁵ and R³⁵ are independently selected from H or fluoro. In another embodiment, R⁸, R⁹ and R³⁵ are independently selected from H or halo and R¹⁰, R¹¹, R¹², R¹³, R¹⁴ and R¹⁵ are each H. In a more particular embodiment, R⁸ and R⁹ are independently selected from H or fluoro and R¹⁰, R¹¹, R¹², R¹³, R¹⁴, R¹⁵ and R³⁵ are each H. In another embodiment, R¹², R¹³ and R³⁵ are independently selected from H or halo and R⁸, R⁹, R¹⁰, R¹¹, R¹⁴ and R¹⁵ are each H. In a more particular embodiment, R¹² and R¹³ are independently selected from H or fluoro and R⁸, R⁹, R¹⁰, R¹¹, R¹⁴, R¹⁵ and R³⁵ are each H. In a more particular embodiment, R⁸, R⁹ R¹⁰, R¹¹, R¹², R¹³, R¹⁴ and R¹⁵ are each H and R³⁵ is H or fluoro.

In particular embodiments of the compound of formula (Iaa) or (Iaaa), tautomer of salt thereof, in which X₂₂ is N, X₁ is CR² wherein R² is H, halogen or C₁₋₄haloalkyl, X₂ and X₃, are both CH, X₄ is CR⁷ wherein R⁷ is hydrogen or halogen, and X₁₅ is C, r is 1 or 2.

In particular embodiments of the compound of formula (Iaa) or (Iaaa), tautomer of salt thereof, in which X₂₂ is N, X₁ is CR² wherein R² is H, halogen or C₁₋₄haloalkyl, X₂ and X₃, are both CH, X₄ is CR⁷ wherein R⁷ is hydrogen or halogen, and X₁₅ is C, a, b and j are not each 1.

In particular embodiments of the compound of formula (Iaa) or (Iaaa), tautomer of salt thereof, in which X₂₂ is N, X₁ is CR² wherein R² is H, halogen or C₁₋₄haloalkyl, X₂ and X₃, are both CH, X₄ is CR⁷ wherein R⁷ is hydrogen or halogen, and X₁₅ is C, r is 1 or 2 and a, b and j are not each 1.

The invention provides a degron which is a compound of formula (Ibb), a tautomer of a compound of formula (Ibb), or a salt thereof:

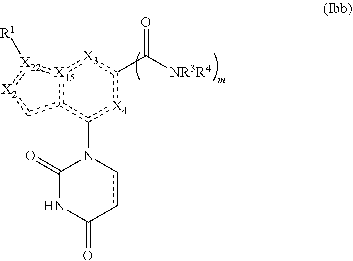

wherein:
R¹ is C₁₋₄alkyl, C₁₋₄haloalkyl or C₁₋₄alkoxy, wherein said C₁₋₄alkyl group is optionally substituted by one C₁₋₄alkoxy group;
X₂ and X₃ are independently selected from N or CH;
X₁₅ and X₂₂ are independently selected from N or C;
X₄ is C—R⁷ or N wherein R⁷ is selected from the group consisting of hydrogen, halogen, C₁₋₃alkyl, C₁₋₃haloalkyl and C₁₋₃alkoxy;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
m is 0 or 1;
n is 0 or 1; and
wherein when X₁₅ is N, then X₂₂ is C.

In one embodiment, the invention provides a degron which is a compound of formula (Ibbb), a tautomer of a compound of formula (Ibbb), or a salt thereof:

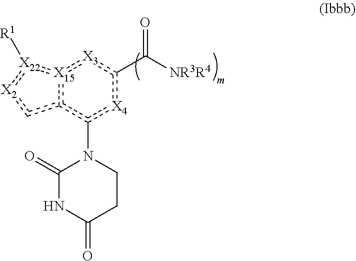

wherein:
R¹ is C₁₋₄alkyl, C₁₋₄haloalkyl or C₁₋₄alkoxy, wherein said C₁₋₄alkyl group is optionally substituted by one C₁₋₄alkoxy group;
X₂ and X₃ are independently selected from N or CH;
X₁₅ and X₂₂ are independently selected from N or C;
X₄ is C—R⁷ or N wherein R⁷ is selected from the group consisting of hydrogen, halogen, C₁₋₃alkyl, C₁₋₃haloalkyl and C₁₋₃alkoxy;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
m is 0 or 1;
n is 0 or 1; and
wherein when X₁₅ is N, then X₂₂ is C.

In one embodiment of the compound of formula (Ibb) or (Ibbb), tautomer, or salt thereof, m is 1. In an alternative embodiment of the compound of formula (Ibb) or (Ibbb), tautomer, or salt thereof, m is 0.

In one embodiment of the compound of formula (Ibb) or (Ibbb), tautomer, or salt thereof, R¹ is C₁₋₄alkyl optionally substituted by by one C₁₋₄alkoxy group. In a more particular embodiment of the compound of formula (Ibb) or (Ibbb), tautomer, or salt thereof, R¹ is C₁₋₄alkyl. In a more particular embodiment, R¹ is methyl, ethyl or isopropyl. In a more particular embodiment, R¹ is ethyl or isopropyl. In one embodiment, R¹ is isopropyl.

In one embodiment of the compound of formula (Ibb) or (Ibbb), tautomer, or salt thereof, X₂₂ is N.

In one embodiment of the compound of formula (Ibb) or (Ibbb), tautomer, or salt thereof, X₂ is CH.

In one embodiment of the compound of formula (Ibb) or (Ibbb), tautomer, or salt thereof, X₃ is CH.

In one embodiment of the compound of formula (Ibb) or (Ibbb), tautomer, or salt thereof, X₁₅ is C.

In one embodiment of the compound of formula (Ibb) or (Ibbb), tautomer or salt thereof, X₄ is C—R⁷ wherein R⁷ is F, Cl or CF₃. In one embodiment, X₄ is C—R⁷ wherein R⁷ is F. In another embodiment, X₄ is CH. In an alternative embodiment, X₄ is N.

In one embodiment of the compound of formula (Ibb) or (Ibbb), tautomer or salt thereof, where X₂₂ is N, X₁ is CR² wherein R² is H, halogen or C₁₋₄haloalkyl, X₂ and X₃, are both CH, X₄ is CR⁷ wherein R⁷ is hydrogen or halogen, and X₁₅ is C. In a more particular embodiment, X₂₂ is N, X₁, X₂, X₃, X₄ are each CH and X₁₅ is C. In another embodiment where X₂₂ and X₂ are N, X₁, is CR² wherein R² is H, halogen or C₁₋₄haloalkyl, X₃ is CH, X₄ is CR⁷ wherein R⁷ is hydrogen or halogen, and X₁₅ is C. In another embodiment where X₂₂ is N, X₁, X₃ and X₄ are each CH, X₁₅ is C and X₂ is N. In a further embodiment where X₂₂ is N, X₁ is N and X₂, X₃ and X₄ are each CH and X₁₅ is C. In an alternative embodiment where X₂₂ is N, X₁ and X₂ are each N and X₃, and X₄ are each CH and X₁₅ is C. In an alternative embodiment where X₂₂ is N, X₂ and X₄ are each N and X₁, and X₃ are each CH and X₁₅ is C. In one embodiment where X₂₂ is N, X₃ and X₄ are each N, is CR² wherein R² is H, halogen, X₂ is CH and X₁₅ is C. In an alternative embodiment where X₂₂ is N, X₃ and X₄ are each N, X₁ and X₂ are each CH and X₁₅ is C. In an alternative embodiment where X₂₂ is N, X₂ and X₃ are each N, X₁ and X₄ are each CH and X₁₅ is C. In an alternative embodiment where X₂₂ is N, X₄ is N, X₁, X₂ and X₃ are each CH and X₁₅ is C. In an alternative embodiment where X₂₂ is N, X₃ is N, X₁, X₂ and X₄ are each CH and X₁₅ is C.

In an alternative embodiment of the compound of formula (Ibb) or (Ibbb), tautomer or salt thereof, X₂ and X₁₅ are N, X₁, X₃, X₄ and are each CH and X₂₂ is C.

In an alternative embodiment of the compound of formula (Ibb) or (Ibbb), tautomer or salt thereof, X₂₂ is C, X₃ and X₄ are each CH, and X₁₅ and X₂ are each N.

In certain embodiments of the compound of formula (Ibb) or (Ibbb), tautomer or salt thereof, R³ is H or C₁₋₄alkyl and R⁴ is (CH₂)ₙR²⁸, wherein R²⁸ is a monocyclic nitrogen containing heterocyclic ring, for example a monocyclic 5-6 membered nitrogen containing heterocyclic ring, more particularly a monocyclic 6 membered nitrogen containing heterocyclic ring. In a more particular embodiment, R³ is C₁₋₄alkyl and R⁴ is —(CH₂)ₙR²⁸, wherein R²⁸ is a monocyclic nitrogen containing heterocyclic ring, for example a monocyclic 5-6 membered nitrogen containing heterocyclic ring, more particularly a monocyclic 6 membered nitrogen containing heterocyclic ring. In an even more particular embodiment, R³ is C₁₋₄alkyl and R⁴ is —(CH₂)ₙR²⁸, wherein R²⁸ is a piperidine ring (e.g. piperdin-4-yl).

In certain embodiments of the compound of formula (Ibb) or (Ibbb), tautomer or salt thereof, R³ and R⁴ together with the nitrogen atom to which they are attached, join together to form a monocyclic nitrogen containing heterocyclic ring. In one embodiment, R³ and R⁴ together with the nitrogen atom to which they are attached, join together to form a monocyclic 5-6 membered nitrogen containing heterocyclic ring, more particularly a monocyclic 6 membered nitrogen containing heterocyclic ring. In an even more particular embodiment, R³ and R⁴ together with the nitrogen atom to which they are attached, join together to form a piperazinyl or piperidinyl ring, more particularly a piperazinyl ring.

In certain embodiments of the compound of formula (Ibb) or (Ibbb), tautomer or salt thereof, R³ and R⁴ together with the nitrogen atom to which they are attached, join together to form a spiro nitrogen containing heterocyclic ring. In one embodiment, R³ and R⁴ together with the nitrogen atom to which they are attached, join together to form a spiro 10-11 membered nitrogen containing heterocyclic ring.

In certain embodiments of the compound of formula (Ibb) or (Ibbb), tautomer or salt thereof, the group NR³R⁴; has a structure selected from the following:

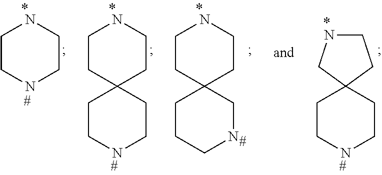

wherein the asterisk represents the position of attachment to the carbonyl group and the # represents the point of attachment to L.

The invention provides a degron which is a compound of formula (Icc), a tautomer of a compound of formula (Icc), or a salt thereof:

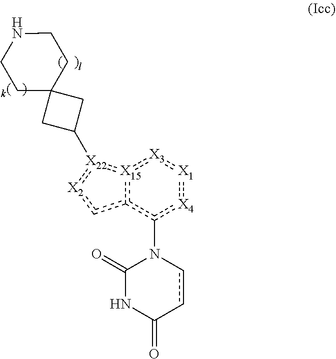

wherein:
X₁ is N or C—R² wherein R² is selected from the group consisting of hydrogen, halogen, C₁₋₄alkyl, C₁₋₄haloalkyl, C₁₋₄alkoxy and —CONR⁵R⁶;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
R⁵ and R⁶ are independently selected from H or C₁₋₄alkyl; and
k and l are independently selected from 0 or 1;
wherein when X₁₅ is N, X₂₂ is C.

In one embodiment, invention provides a degron which is a compound of formula (Iccc), a tautomer of a compound of formula (Icc), or a salt thereof:

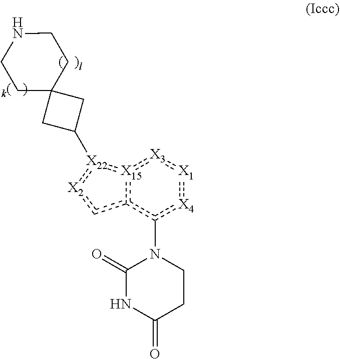

wherein:
X₁ is N or C—R² wherein R² is selected from the group consisting of hydrogen, halogen, C₁₋₄alkyl, C₁₋₄haloalkyl, C₁₋₄alkoxy and —CONR⁵R⁶;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
R⁵ and R⁶ are independently selected from H or C₁₋₄alkyl; and
k and l are independently selected from 0 or 1;
wherein when X₁₅ is N, X₂₂ is C.

In one embodiment of the compound of formula (Icc) or (Iccc), tautomer, or salt thereof, k and l are each 1.

In another embodiment of the compound of formula (Icc) or (Iccc), tautomer, or salt thereof, k and l are each 0.

In one embodiment of the compound of formula (Icc) or (Iccc), tautomer, or salt thereof, X₂₂ is N. In certain embodiments in which X₂₂ is N, one or more of X₁, X₂, X₃, X₄, are additionally N.

In one embodiment of the compound of formula (Icc) or (Iccc), tautomer, or salt thereof, X₁₅ is C.

In one embodiment of the compound of formula (Icc) or (Iccc), tautomer or salt thereof, X₁ is N or CH.

In another embodiment of formula (Icc) or (Iccc), tautomer or salt thereof, X₁ is C—R² wherein R² is selected from the group consisting of CF₃, F or Cl. In a more particular embodiment, X₁ is C—R² wherein R² is selected from the group consisting of F or Cl.

In one embodiment of the compound of formula (Icc) or (Iccc), tautomer or salt thereof, X₄ is C—R⁷. In a more particular embodiment, X₄ is C—R⁷ wherein R⁷ is F, Cl or CF₃. In one embodiment, X₄ is C—R⁷ wherein R⁷ is F. In another embodiment, X₄ is CH. In an alternative embodiment, X₄ is N.

In one embodiment of the compound of formula (Icc) or (Iccc), tautomer or salt thereof, R² is selected from hydrogen and halo.

In one embodiment of the compound of formula (Icc) or (Iccc), tautomer or salt thereof, X₁ is N or CH.

In another embodiment of formula (Icc) or (Iccc), tautomer or salt thereof, X₁ is C—R² wherein R² is selected from the group consisting of CF₃, F or Cl. In a more particular embodiment, X₁ is C—R² wherein R² is selected from the group consisting of F or Cl.

In one embodiment of the compound of formula (Icc) or (Iccc), tautomer or salt thereof, X₄ is C—R⁷. In a more particular embodiment, X₄ is C—R⁷ wherein R⁷ is F, Cl or CF₃. In one embodiment, X₄ is C—R⁷ wherein R⁷ is F. In another embodiment, X₄ is CH. In an alternative embodiment, X₄ is N.

In one embodiment of the compound of formula (Icc) or (Iccc), tautomer or salt thereof, X₂₂ is N, X₁ is CR² wherein R² is H, halogen or C₁₋₄haloalkyl, X₂ and X₃, are both CH, X₄ is CR⁷ wherein R⁷ is hydrogen or halogen, and X₁₅ is C. In a more particular embodiment, X₂₂ is N, X₁, X₂, X₃, X₄ are each CH and X₁₅ is C. In another embodiment where X₂₂ and X₂ are N, X₁, is CR² wherein R² is H, halogen or C₁₋₄haloalkyl, X₃ is CH, X₄ is CR⁷ wherein R⁷ is hydrogen or halogen, and X₁₅ is C. In another embodiment where X₂₂ is N, X₁, X₃ and X₄ are each CH, X₁₅ is C and X₂ is N. In a further embodiment where X₂₂ is N, X₁ is N and X₂, X₃ and X₄ are each CH and X₁₅ is C. In an alternative embodiment where X₂₂ is N, X₁ and X₂ are each N and X₃, and X₄ are each CH and X₁₅ is C. In an alternative embodiment where X₂₂ is N, X₂ and X₄ are each N and X₁, and X₃ are each CH and X₁₅ is C. In one embodiment where X₂₂ is N, X₃ and X₄ are each N, is CR² wherein R² is H, halogen, X₂ is CH and X₁₅ is C. In an alternative embodiment where X₂₂ is N, X₃ and X₄ are each N, X₁ and X₂ are each CH and X₁₅ is C. In an alternative embodiment where X₂₂ is N, X₂ and X₃ are each N, X₁ and X₄ are each CH and X₁₅ is C. In an alternative embodiment where X₂₂ is N, X₄ is N, X₁, X₂ and X₃ are each CH and X₁₅ is C. In an alternative embodiment where X₂₂ is N, X₃ is N, X₁, X₂ and X₄ are each CH and X₁₅ is C.

In an alternative embodiment of the compound of formula (Icc) or (Iccc), tautomer or salt thereof, X₂ and X₁₅ are N, X₁, X₃, X₄ are each CH and X₂₂ is C.

### Target Binding Moiety

In one embodiment, the invention relates to PROTACs using the novel degron of the invention and the nature of the target is not limited, except in the fact that it must be an intracellular protein or at least be a protein comprising an intracellular domain. Many proteins of therapeutic importance are well studied with a plethora of compounds known that are capable of binding to these. These compounds may be utilised as the target binding moiety in PROTAC compounds. The Examples include compounds in which the target binding moiety is an androgen receptor binding moiety, a RIPK2 binding moiety and and IRAK4 binding moiety. Degradation of each of these targets has been demonstrated. This is evidence that the nature of the target is not limited, and that the degrons of the invention can be utilised to mediate degradation of a wide variety of targets.

In one embodiment, the target is the androgen receptor. Compounds binding the androgen receptor are well known in the art. In one embodiment, the androgen receptor binding moiety has the structure of formula (III)

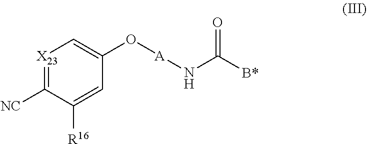

wherein the attachment position to L is shown by the asterisk;
A is selected from the group consisting of: cyclohexyl, cyclobutyl or a 6 membered nitrogen containing heterocyclic ring, wherein said cyclohexyl, cyclobutyl or 6 membered nitrogen containing heterocyclic ring may be optionally substituted with up to 4 C₁₋₄alkyl groups;
B is selected from the group consisting of phenyl, a 6 membered nitrogen containing heteroaryl group or a fused bicyclic nitrogen containing heterocyclic ring, wherein B is optionally substituted by one or more halogen groups;
X₂₃ is CH or N, and
R¹⁶ is selected from the group consisting of halo or CF₃.

In one embodiment, X₂₃ is CH.

In one embodiment, X₂₃ is N.

In one embodiment where the androgen receptor binding moiety has the structure of formula (III), A is selected from the group consisting of: cyclohexyl or cyclobutyl, wherein said cyclohexyl or cyclobutyl may be optionally substituted with up to 4 C₁₋₄alkyl groups. In one embodiment where the androgen binding moiety has the structure of formula (III), A is unsubstituted cyclohexyl. In another embodiment where the androgen binding moiety has the structure of formula (III), A is cyclobutyl substituted with 4 C₁₋₄alkyl groups. In another embodiment where the androgen binding moiety has the structure of formula (III), A is cyclobutyl substituted with 4 methyl groups.

In one embodiment where the androgen receptor binding moiety has the structure of formula (III), B is selected from phenyl or a 6 membered nitrogen containing heteroaryl group wherein said phenyl or 6 membered nitrogen containing heteroaryl group is optionally substituted by one or more halogen groups. In one embodiment, B is selected from phenyl or a 6 membered nitrogen containing heteroaryl group wherein said phenyl or 6 membered nitrogen containing heteroaryl group is unsubstituted. In one embodiment, B is phenyl which is optionally substituted by one or more halogen groups. In one embodiment, B is phenyl which is substituted by one or more fluoro groups. In one embodiment, B is unsubstituted phenyl. In an alternative embodiment, B is selected from a 6 membered nitrogen containing heteroaryl group which is optionally substituted by one or more halogen groups. In one embodiment, B is an unsubstituted 6 membered nitrogen containing heteroaryl group. In a more particular embodiment, B is selected from pyridinyl, pyridazinyl pyrimidinyl and pyrazinyl. In an even more particular embodiment, B is selected from pyridazin-3-yl, pyrimidin-5-yl, pyrazin-2-yl and pyridin-3-yl wherein the numbering represents the attachment position to the carbonyl group.

In an alternative embodiment where the androgen receptor binding moiety has the structure of formula (III), B is a fused bicyclic nitrogen containing heterocyclylic ring which is optionally substituted by one or more halogen groups. In a more particular embodiment, B is a 9-10 membered fused bicyclic nitrogen containing heterocyclylic ring which is optionally substituted by one or more halogen groups. In one embodiment where the androgen receptor binding moiety has the structure of formula (III), B is a 9-10 membered fused bicyclic nitrogen containing heterocyclylic ring which is unsubstituted. In particular embodiments, B has the structure set out below (where * represents the attachment to the carbonyl group and # represents the attachment to the linker):

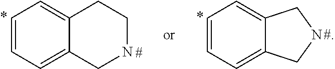 [SMILES: c1ccc2c(c1)CCNC2 ; c1ccc2c(c1)CNC2]

In one embodiment of the androgen receptor binding moiety of formula (III), R¹⁶ is chloro or CF₃.

In a more particular embodiment, the androgen receptor binding moiety has the structure of formula (IIIa)

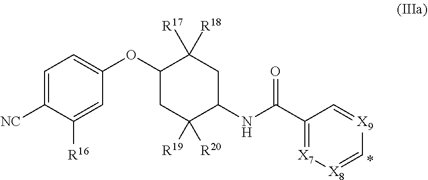

wherein the attachment position to L is shown by the asterisk;
X₇, X₈ and X₉ are independently CH, C—F or N;
R¹⁶ is selected from the group consisting of halo or CF₃;
R¹⁷, R¹⁸, R¹⁹ and R²⁰ are independently H or C₁₋₄alkyl.

In particular embodiments of the androgen receptor binding moiety of formula (IIIa), R¹⁶ is chloro or CF₃. In a particular embodiment of the androgen receptor binding moiety of formula (IIIa), R¹⁶ is chloro.

In particular embodiments of the androgen receptor binding moiety of formula (IIIa), R¹⁷, R¹⁸, R¹⁹ and R²⁰ are H or methyl. In one embodiment, R¹⁷ and R¹⁹ are methyl, and R¹⁸ and R²⁰ are H. In one embodiment, R¹⁷, R¹⁸, R¹⁹ and R²⁰ are each H.

In particular embodiments of the androgen receptor binding moiety of formula (IIIa), either:
X₇ and X₈ are N and X₉ is CH; or
X₈ and X₉ are N and X₇ is CH; or
X₇ and X₉ are N and X₈ is CH; or
X₇ is N and X₈ and X₉ are CH; or
X₈ is N and X₇ and X₉ are CH; or
X₇ is C—F and X₈ and X₉ are CH; or
X₇, X₈ and X₉ are each CH.

In more particular embodiments of the androgen receptor binding moiety of formula (IIIa), X₇ and X₈ are N and X₉ is CH. In particular embodiments in which X₇ and X₈ are N and X₉ is CH, R¹⁷, R¹⁸, R¹⁹ and R²⁰ are each H.

In more particular embodiments of the androgen receptor binding moiety of formula (IIIa), X₇ and X₉ are N and X₈ is CH. In particular embodiments in which X₇ and X₉ are N and X₈ is CH, R¹⁷, R¹⁸, R¹⁹ and R²⁰ are each H.

In an alternative embodiment, the androgen receptor binding moiety has the structure of formula (IIIb):

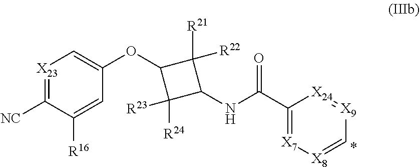

wherein the attachment position to L is shown by the asterisk;
X₇, X₈ and X₉ are independently CH, C—F or N;
X₂₃ and X₂₄ are independently CH or N;
R¹⁶ is selected from the group consisting of halo or CF₃; and
R²¹, R²², R²³ and R²⁴ are independently H or C₁₋₄alkyl.

The androgen receptor binding moiety of formula (IIIb) can bind to certain mutated versions of the androgen receptor. Protacs containing the androgen receptor binding moiety of formula (IIIb) exhibit activity in the dual mutant (T878A/L702H) Androgen Receptor Degradation Assay. Such protacs are expected to be useful for the treatment of castration resistant prostate cancer.

In one embodiment, X₂₃ is CH.

In one embodiment, X₂₃ is N.

In particular embodiments of the androgen receptor binding moiety of formula (IIIb), R¹⁶ is chloro or CF₃. In a particular embodiment of the androgen receptor binding moiety of formula (IIIb), R¹⁶ is chloro.

In particular embodiments of the androgen receptor binding moiety of formula (IIIb), R²¹, R²², R²³ and R²⁴ are each methyl.

In particular embodiments of the androgen receptor binding moiety of formula (IIIb), either:
X₇ and X₈ are N and X₉ and X₂₄ are CH; or
X₈ and X₉ are N and X₇ and X₂₄ are CH; or
X₇ and X₉ are N and X₈ and X₂₄ are CH; or
X₇ is N and X₈, X₉ and X₂₄ are CH; or
X₈ is N and X₇, X₉ and X₂₄ are CH; or
X₇ is C—F and X₈, X₉ and X₂₄ are CH; or
X₇, X₈, X₉ and X₂₄ are each CH; or
X₇ and X₂₄ are N and X₈ and X₉ are CH.

In a more particular embodiment of the androgen receptor binding moiety of formula (IIIb), X₂₃ is N and either:
X₇ and X₈ are N and X₉ and X₂₄ are CH; or
X₈ and X₉ are N and X₇ and X₂₄ are CH; or
X₇ is N and X₈, X₉ and X₂₄ are CH; or
X₇ and X₂₄ are N and X₈ and X₉ are CH.

In an even more particular embodiment of the androgen receptor binding moiety of formula (IIIb), X₂₃ is N, R²¹, R²², R²³ and R²⁴ are each methyl, R¹⁶ is chloro or CF₃ and either:
X₇ and X₈ are N and X₉ and X₂₄ are CH; or
X₈ and X₉ are N and X₇ and X₂₄ are CH; or
X₇ is N and X₈, X₉ and X₂₄ are CH; or
X₇ and X₂₄ are N and X₈ and X₉ are CH.

In an alternative embodiment, the androgen receptor binding moiety has the structure of formula (IIIc):

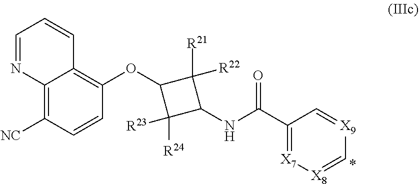

wherein the attachment position to L is shown by the asterisk;
X₇, X₈ and X₉ are independently CH, C—F or N;
R²¹, R²², R²³ and R²⁴ are independently H or C₁₋₄alkyl.

In particular embodiments of the androgen receptor binding moiety of formula (IIIc), R²¹, R²², R²³ and R²⁴ are each methyl.

In particular embodiments of the androgen receptor binding moiety of formula (IIIc), either:
X₇ and X₈ are N and X₉ is CH; or
X₈ and X₉ are N and X₇ is CH; or
X₇ and X₉ are N and X₈ is CH; or
X₇ is N and X₈ and X₉ are CH; or
X₈ is N and X₇ and X₉ are CH; or
X₇ is C—F and X₈ and X₉ are CH; or
X₇, X₈ and X₉ are each CH.

In a more particular embodiment of the androgen receptor binding moiety of formula (IIIc), either:
X₇ and X₈ are N and X₉ is CH; or
X₇ is N and X₈ and X₉ are CH; or
X₈ is N and X₇ and X₉ are CH.

In an alternative embodiment, the androgen receptor binding moiety has the structure of formula (IV):

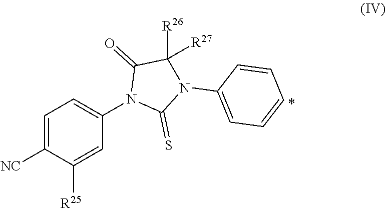

wherein the attachment position to L is shown by the asterisk;
R²⁵ is selected from the group consisting of halo or CF₃; and
R²⁶ and R²⁷ are independently H or C₁₋₄alkyl.

In particular embodiments of the androgen receptor binding moiety of formula (IV), R²⁵ is chloro or CF₃. In a more particular embodiment, R²⁵ is CF₃.

In particular embodiments of the androgen receptor binding moiety of formula (IV), R²⁶ and R²⁷ are each methyl.

In an alternative embodiment, the androgen receptor binding moiety has the structure of formula (V):

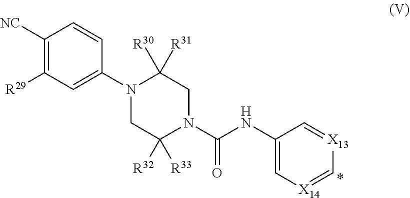

wherein the attachment position to L is shown by the asterisk;
X₁₃ and X₁₄ are independently CH or N;
R²⁹ is selected from the group consisting of halo or CF₃;
R³⁰, R³¹, R³² and R³³ are independently H or C₁₋₄alkyl.

In particular embodiments of the androgen receptor binding moiety of formula (V), R²⁹ is chloro or CF₃. In a more particular embodiment of the androgen receptor binding moiety of formula (VI), R²⁹ is chloro.

In particular embodiments of the androgen receptor binding moiety of formula (V), R³⁰, R³¹, R³² and R³³ are H or methyl. In one embodiment, R³⁰ and R³² are methyl, and R³¹ and R³³ are H. In one embodiment, R³⁰, R³¹, R³² and R³³ are each H.

In particular embodiments of the androgen receptor binding moiety of formula (V), either:
X₁₃ and X₁₄ are N; or
X₁₃ is N and X₁₄ is CH.

### Linker

L is a chemical linker. Linkers for PROTACs are well known in the art and can be chemically diverse. In one embodiment, L is a chemical linker group containing between 1-50 atoms in total. In more particular embodiments, L contains between 1 and 30 atoms, between 1-20 atoms, and between 10 and 20 atoms. In one embodiment, L is a hydrocarbon chain wherein one or more carbon atoms are replaced by —O—, —NH—, —NCH₃—, —CO—, phenylene, 5-6 membered heteroarylene, C₄-6cycloalkylene and -4-6 membered heterocyclylene, wherein the carbon atoms in said hydrocarbon chain or in said phenyl, 5-6 membered heteroaryl, C₄-6cycloalkyl and -4-6 membered heterocyclylic rings are optionally substituted by one or more substituents selected from the group consisting of oxo, C₁₋₃ alkyl, C₁₋₃ alkoxy, OH, halogen, NH₂, NH(C₁₋₃ alkyl), N(C₁₋₃ alkyl)₂ or CN.

Suitable L groups are well known in the art. Suitable L groups include those described in, for example, WO2017197055, WO2017007612, WO2015160845, WO2021077010, WO2018071606, WO2020211822, WO2020198711, WO2020160295 and WO2020214952. In addition, a wide range of linkers suitable for PROTAC development are commercially available from vendors including Selleck Chemicals, BroadPharm and MedChemExpress.com.

Whilst the PROTAC approach is a principle capable of general application, such that a PROTAC capable of degrading a particular target using a validated target binding moiety may be found without undue burden using linkers known in the art, it has become clear that not all PROTACs have equivalent activity in vitro or in vivo. There are several reasons for this including differential absorption through the intestinal tract, differential absorption into the cells, differential pharmacokinetics and the differential ability of the PROTAC molecule to form a functional ternary complex (a pre-requisite to ubiquitination and degradation of the target). PROTACs can be optimised by selection of different target binding moieties, E3 ligase binders and also different linkers. Whilst a variety of linkers are expected to result in at least some degradation of the target, the length and chemical nature of L can be optimised for specific TBMs according to methods known in the art. In some cases, flexible linkers may be optimal, whilst for other TBMs, more rigid linkers that maintain the relative positions of key functional groups within the target binding moiety and degron are suitable.

When the target binding moiety is an androgen receptor binding moiety of formula (III), (IIIa), (IIIb), (IIIc), (IV) or (V), L is a group of formula (VI):

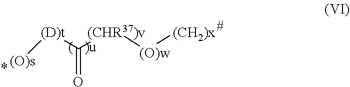

wherein * represents the attachment to the androgen receptor binding moiety, and # represents the attachment to the compound of formula (I);
D is a nitrogen containing heterocyclic ring which nitrogen containing heterocyclic ring is optionally substituted with one or more substituents selected from the group consisting of methyl, halo, CF₃ and CN;
R³⁷ is methyl or hydrogen;
s is 0 or 1;
t is 0 or 1;
u is 0 or 1;
v is 0, 1 or 2;
w is 0 or 1;
x is 0, 1 2, 3 or 4
wherein when v is 0, u and w are not both 1;
wherein when t is 0, s and u are not both 1.

In one embodiment, D is a 4-6 membered monocyclic nitrogen containing heterocyclic ring, which 4-6 membered monocyclic nitrogen containing heterocyclic ring is optionally substituted with one or more substituents selected from the group consisting of methyl, halo, CF₃ and CN.

In an alternative embodiment, D is a spiro nitrogen containing heterocyclic ring, which spiro nitrogen containing heterocyclic ring is optionally substituted with one or more substituents selected from the group consisting of methyl, halo, CF₃ and CN.

In an alternative embodiment, D is a bridged or fused nitrogen containing heterocyclic ring, which bridged or fused nitrogen containing heterocyclic ring is optionally substituted with one or more substituents selected from the group consisting of methyl, halo, CF₃ and CN. In one embodiment, the bridged or fused heterocyclic ring has a structure selected from the following:

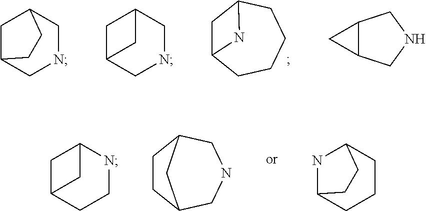

In a more particular embodiment, the bridged or fused heterocyclic ring has a structure selected from the following:

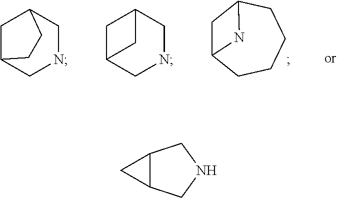

In one embodiment, R³⁷ is hydrogen.

In particular embodiments, L is a group of formula (VIa):

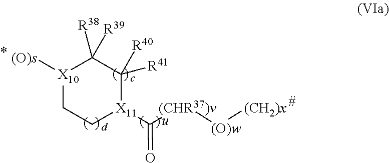

wherein * represents the attachment to the androgen receptor binding moiety, and # represents the attachment to the compound of formula (I);
X₁₀ is CH or N;
X₁₁ is a CR⁴² or N;
c is 0 or 1;
d is 0 or 1;
s is 0 or 1;
u is 0 or 1;
v is 0, 1 or 2;
w is 0 or 1;
x is 0, 1 or 2;
R³⁷ is H or methyl; and
R³⁸, R³⁹, R⁴⁰, R⁴¹ and R⁴² are independently selected from H, halo, methyl, CF₃ and CN.

In particular embodiments of the group of formula (VIa):
c and d are each 0; or
c is 1 and d is 0; or
c and d are each 1.

In one embodiment of the group of formula (VIa), R³⁷ is H. In an alternative embodiment, R³⁷ is methyl.

In one embodiment of the group of formula (VIa), R⁴² is hydrogen, methyl, fluoro or CN. In a more particular embodiment, R⁴² is hydrogen.

In one embodiment of the group of formula (VIa), R³⁸, R³⁹, R⁴⁰ and R⁴¹ are independently selected from H, CF₃ and methyl. In a more particular embodiment, R³⁸, R³⁹, R⁴⁰ and R⁴¹ are independently selected from H and methyl. In a more particular embodiment, R³⁸, R³⁹, R⁴⁰ and R⁴¹ are each H.

In particular embodiment of formula (VIa):
X₁₀ is N and X₁₁ is CR⁴² or N;
c is 0 or 1;
d is 0 or 1;
s is 0;
u is 0 or 1; and
v is 0, 1 or 2;
w is 0 or 1;
x is 0, 1 or 2; and
R³⁷, R³⁸, R³⁹, R⁴⁰, R⁴¹ and R⁴² are independently selected from H or methyl.

In more particular embodiment of formula (VIa):
X₁₀ is N and X₁₁ is CR⁴²;
c is 1;
d is 1;
s is 0;
u is 0 or 1; and
v is 0, 1 or 2;
w is 0 or 1;
x is an integer between 0 and 2; and
R³⁷, R³⁸, R³⁹, R⁴⁰, R⁴¹ and R⁴² are independently selected from H or methyl.

In more particular embodiment of formula (VIa):
X₁₀ is N and X₁₁ is CR⁴²;
c is 1;
d is 1;
s is 0;
u is 0 or 1; and
v is 0, 1 or 2;
w is 0;
x is 0; and
R³⁷, R³⁸, R³⁹, R⁴⁰, R⁴¹ and R⁴² are independently selected from H or methyl.

In more particular embodiment of formula (VIa) where X₁₀ is N and X₁₁ is CR⁴², c is 1, d is 1; s is 0, u is 1, v is 0, w is 0, x is 0; and R³⁸, R³⁹, R⁴⁰, R⁴¹ and R⁴² are independently selected from H or methyl.

In more particular embodiment of formula (VIa) where X₁₀ is N and X₁₁ is CR⁴² c is 1, d is 1, s is 0, u is 0, v is an integer between 0 and 2, w is 0, x is 0 and R³⁷, R³⁸, R³⁹, R⁴⁰, R⁴¹ and R⁴² are independently selected from H or methyl. In one embodiment, v is 0. In another embodiment, v is 1. In a further embodiment, v is 2.

In more particular embodiment of formula (VIa), X₁₀ is N and X₁₁ is CR⁴², c is 1, d is 1, s is 0, u is 0,
v is 1, w is 0, x is 0 and R³⁷, R³⁸, R³⁹, R⁴⁰, R⁴¹ and R⁴² are each H.

In a different embodiment of formula (VIa):
X₁₀ is CH and X₁₁ is CR⁴² or N;
c is 1;
d is 1;
s is 0 or 1;
u is 0 or 1;
v is 0 or 1;
w is 0;
x is 0; and
R³⁷, R³⁸, R³⁹, R⁴⁰, R⁴¹ and R⁴² are independently selected from H or methyl.

In more particular embodiment:
X₁₀ is CH and X₁₁ is N;
c is 1;
d is 1;
s is 0 or 1; and
u and v are each 1;
w is 0;
x is 0; and
R³⁷, R³⁸, R³⁹, R⁴⁰ and R⁴¹ are independently selected from H or methyl.

In an alternative embodiment of formula (VIa), X₁₀ and X₁₁ are each CH, c and d are each 1, s, u, v, w and x are each 0, and R³⁸, R³⁹, R⁴⁰ and R⁴¹ are independently selected from H or methyl.

In an alternative embodiment of formula (VIa):
X₁₀ is N and X₁₁ is N;
c is 1;
d is 1;
s is 0;
u is 0; and
v is 1 or 2;
w is 1; and
x is 1 or 2; and
R³⁷, R³⁸, R³⁹, R⁴⁰ and R⁴¹ are independently selected from H or methyl.

In a more particular embodiment where X₁₀ is N and X₁₁ is N, c and d are 1, s and u are 0, v is 2, w is 1, x is 2, and R³⁷, R³⁸, R³⁹, R⁴⁰ and R⁴¹ are independently selected from H or methyl.

In particular embodiments, L is a group of formula (VIb):

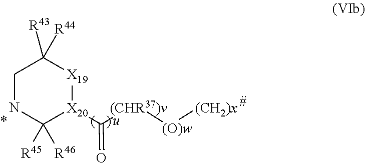

wherein * represents the attachment to the androgen receptor binding moiety, and # represents the attachment to the compound of formula (I);
X₁₉ is CH or O;
X₂₀ is CR⁴⁷;
u is 0 or 1;
v is 0, 1 or 2;
w is 0 or 1;
x is 0, 1 or 2;
R³⁷ is H or methyl; and
R⁴³, R⁴⁴, R⁴⁵, R⁴⁶ and R⁴⁷ are independently selected from H, halo, methyl, CF₃ and CN.

In one embodiment of the group of formula (VIb), R³⁷ is H. In an alternative embodiment, R³⁷ is methyl.

In one embodiment of the group of formula (VIb), R⁴⁷ is hydrogen, methyl, fluoro or CN. In a more particular embodiment, R⁴⁷ is hydrogen.

In one embodiment of the group of formula (VIb), R⁴³, R⁴⁴, R⁴⁵ and R⁴⁶ are independently selected from H, CF₃ and methyl. In a more particular embodiment, R⁴³, R⁴⁴, R⁴⁵ and R⁴⁶ are independently selected from H and methyl. In a more particular embodiment, R⁴³, R⁴⁴, R⁴⁵ and R⁴⁶ are each H.

In particular embodiment of formula (VIb):
X₁₉ is CH or O;
X₂₀ is CH;
u is 0; and
v is 0, 1 or 2;
w is 0;
x is 0; and
R⁴³, R⁴⁴, R⁴⁵ and R⁴⁶ are independently selected from H and methyl.

In particular embodiments, L is a group of formula (VIc):

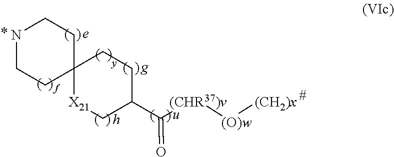

wherein * represents the attachment to the androgen receptor binding moiety, and # represents the attachment to the compound of formula (I);
X₂₁ is CH₂ or O;
e is 0 or 1;
f is 0 or 1;
g is 0 or 1;
h is 0 or 1;
u is 0 or 1;
v is 0, 1 or 2;
w is 0 or 1;
x is 0, 1 or 2;
y is 0 or 1; and
R³⁷ is H or methyl; and
wherein at least one of g h and y is 1.

In one embodiment of the group of formula (VIc), R³⁷ is H. In an alternative embodiment, R³⁷ is methyl.

In one embodiment of the group of formula (VIc), X₂₁ is CH₂.

In particular embodiments of the group of formula (VIc):
e, f, g, h and y are each 1; or
e and f are 0 and g, h and y are 1; or
e, f, g and h are 0, and y is; or
e, g and h are 0 and f and y are 1; or
e, f, g and y are 0 and h is 1.

In one embodiment of the group of formula (VIc), u, w and x are each 0 and v is 0 or 1.

In particular embodiments, L is a group of formula (VId):

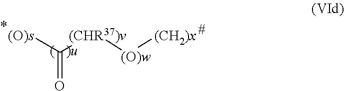

wherein * represents the attachment to the androgen receptor binding moiety, and # represents the attachment to the compound of formula (I);
s is 0 or 1;
u is 0 or 1;
v is 0, 1 or 2;
x is 0, 1 2, 3 or 4; and
R³⁷ is H or methyl;
wherein when u is 1, is is 0; and
wherein when v is 0, u and w are not both 1.

In a particular embodiment of formula (VId), s and w are both 0 and u is 1. In more particular embodiments, the sum of v and x is between 1 and 4, such that L is either *CO(CH₂)₁₋₄ #. In a more particular embodiment, L is *CO(CH₂)#.

In a particular embodiment of formula (VId), s is 1 and u and w are both 0. In more particular embodiments, the sum of v and x is 3 or 4, such that L is either *O(CH₂)₃ # or *O(CH₂)₄ #.

In a particular embodiment of formula (VId), s, u and w are each 0. In more particular embodiments, the sum of v and x is between 1 and 6, such that L is *(CH₂)₁₋₆ #. In more particular embodiments, L is (CH₂)₄, (CH₂)₅ or (CH₂)₆.

In certain embodiments, linkers of formula (VId) as described herein may be used in compounds of formula (Ib) in which R³ and R⁴ together with the nitrogen atom to which they are attached, join together to form a spiro nitrogen containing heterocyclic ring. More particularly, linkers of formula (VId) as described herein may be used in compounds of formula (Ib) in which R³ and R⁴ together with the nitrogen atom to which they are attached, join together to form a spiro nitrogen containing heterocyclic ring having a structure selected from the group consisting of:

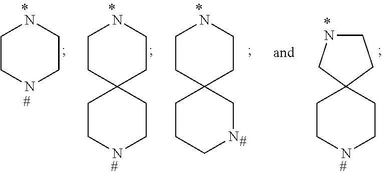

wherein the asterisk represents the position of attachment to the carbonyl group and the # represents the point of attachment to L.

In certain embodiments, linkers of formula (VId) as described herein may be used in compounds of formula (I), (Ia) and (Ib) in which the androgen binding has the structure of formula (III) wherein B is a fused bicyclic nitrogen containing heterocyclyl ring. In particular embodiments, B has the structure set out below (where * represents the attachment to the carbonyl group and # represents the attachment to the linker):

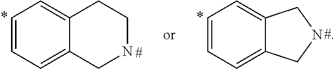 [SMILES: c1ccc2c(c1)CCNC2 ; c1ccc2c(c1)CNC2]

When the target binding moiety is an androgen receptor binding moiety of formula (III), (IIIa), (IIIb), (IV) or (V), L may be a group of formula *(CH₂)zN(CH₃)#, wherein * represents the attachment to the androgen receptor binding moiety, and # represents the attachment to the compound of formula (I) and wherein z is 1, 2, 3, 4, or 5.

### PROTACs

Compounds of formula (I), (Ia) and (Ib) in which q is 1 are “PROTACs”. The nature of the linker present in the PROTAC is determined by p. Where p is 0, there is no linker (or, alternatively, the linker could be considered to be a bond). Where p is 1, the linker is as defined herein for L. Compounds in which p is 1 but q is 0 are compounds comprising a degron and a linker. Such compounds are useful as intermediates in the manufacture of PROTACs.

Whilst it is convenient to mentally divide up the PROTAC compounds of the invention as being comprised of target binding moieties, linkers and a cereblon binding moiety or degron, it is appreciated that these are not discrete moieties and impact upon one another due to the conformation adopted by the compound as a whole. For example, it is possible that a portion of the compound that falls under the definition of L may, in fact bind to either the target binding moiety or to cereblon, depending upon the conformation of the compound as a whole. Conversely, it is possible that a part of the cereblon binding moiety may not in fact be involved in binding cereblon due to the conformation of the compound as a whole. The skilled person will appreciate that the designation of a part of the molecule as a linker, cereblon binder or target binder (androgen binding moiety) is simply for convenience and is not intended to limit the function of these portions of the compounds.

Compounds of formula (I), tautomers, or salts thereof where q is 1 and the target binding moiety is an androgen binding moiety of formula (III), (IIIa), (IIIb), (IV) or (V) are androgen receptor PROTACs (or salts thereof). In particular embodiments, the compound is a compound of formula (Ia), a tautomer or a salt thereof. In more particular embodiments, the compound is a compound of formula (Ia), a tautomer or a salt thereof, and has an androgen binding moiety of formula (IIIa).

In particular embodiments of the androgen receptor binding moiety of formula (IIIa), R¹⁶ is chloro or CF₃.

In particular embodiments of the androgen receptor binding moiety of formula (IIIa), R¹⁷, R¹⁸, R¹⁹ and R²⁰ are H or methyl. In one embodiment, R¹⁷ and R¹⁹ are methyl, and R¹⁸ and R²⁰ are H. In one embodiment, R¹⁷, R¹⁸, R¹⁹ and R²⁰ are each H.

In particular embodiments of the androgen receptor binding moiety of formula (IIIa), either:
X₇ and X₈ are N and X₉ is CH; or
X₈ and X₉ are N and X₇ is CH; or
X₇ and X₉ are N and X₈ is CH; or
X₇ is N and X₈ and X₉ are CH; or
X₈ is N and X₇ and X₉ are CH; or
X₇ is C—F and X₈ and X₉ are CH; or
X₇, X₈ and X₉ are each CH.

In more particular embodiments of the androgen receptor binding moiety of formula (IIIa), X₇ and X₈ are N and X₉ is CH. In particular embodiments in which X₇ and X₈ are N and X₉ is CH, R¹⁷, R¹⁸, R¹⁹ and R²⁰ are each H.

In particular embodiments, the invention provides a compound of formula (VII), a tautomer of a compound of formula (VII), or a salt thereof:

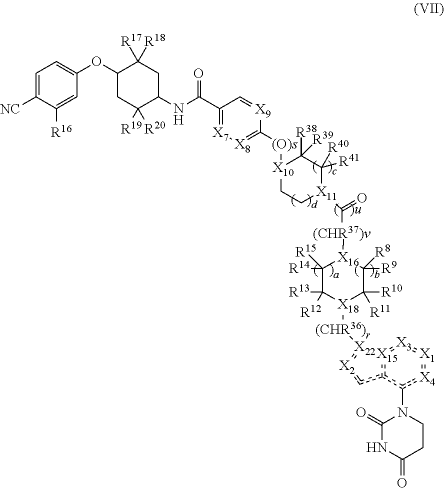

wherein:
   X₁ is N or C—R² wherein R² is selected from the group consisting of hydrogen, halogen, C₁₋₄alkyl, C₁₋₄haloalkyl, C₁₋₄alkoxy and —CONR⁵R⁶;
   X₄ is C—R⁷ or N wherein R⁷ is selected from the group consisting of hydrogen, halogen, C₁₋₃alkyl, C₁₋₃haloalkyl and C₁₋₃alkoxy;
   X₂ and X₃ are independently selected from N or CH;
   X₁₅ and X₂₂ are independently selected from N or C;
   X₁₆ is N or CH;
   X₁₈ is CR³⁵, wherein R³⁵ is H or halo;
   a and b are independently 0 or 1;
   r is 0 or 1;
   R⁵ and R⁶ are independently selected from H or or C₁₋₄alkyl;
   R⁸, R⁹, R¹⁰, R¹¹, R¹², R¹³, R¹⁴, R¹⁵ and R³⁵ are independently selected from H or halo;
   R³⁶ is hydrogen or methyl.
   X₁₀ is CH or N;
   X₁₁ is CR³⁴ or N wherein R³⁴ is hydrogen or halo;
   c is 0 or 1;
   d is 0 or 1;
   R³⁷ is H or methyl;
   R³⁸, R³⁹, R⁴⁰, R⁴¹ and R⁴² are independently selected from H, halo, methyl, CF₃ and CN;
   s is 0 or 1;
   u is 0 or 1; and
   v is 0, 1 or 2;
   X₇, X₈ and X₉ are independently CH, C—F or N;
   R¹⁶ is selected from the group consisting of halo or CF₃;
   R¹⁷, R¹⁸, R¹⁹ and R²⁰ are independently H or C₁₋₄alkyl; and
   wherein when X₁₅ is N, X₂₂ is CH.

In one embodiment of the compound of formula (VII), tautomer or salt thereof, R² is selected from hydrogen and halo.

In one embodiment of the compound of formula (VII), tautomer or salt thereof, X₁ is N or CH.

In another embodiment of formula (VII), tautomer or salt thereof, X₁ is C—R² wherein R² is selected from the group consisting of CF₃, F or Cl. In a more particular embodiment, X₁ is C—R² wherein R² is selected from the group consisting of F or Cl.

In one embodiment of the compound of formula (VII), tautomer or salt thereof, X₄ is C—R⁷. In a more particular embodiment, X₄ is C—R⁷ wherein R⁷ is F, Cl or CF₃. In one embodiment, X₄ is C—R⁷ wherein R⁷ is F. In another embodiment, X₄ is CH. In an alternative embodiment, X₄ is N.

In one embodiment of the compound of formula (VII), tautomer or salt thereof, X₂₂ is N.

In one embodiment of the compound of formula (VII), tautomer or salt thereof, X₁₅ is C.

In particular embodiments where X₂₂ is N, X₁, X₂, X₃, X₄ are each CH and X₁₅ is C. In another embodiment where X₂₂ is N, X₁, X₃ and X₄ are each CH, X₁₅ is C and X₂ is N. In a further embodiment where X₂₂ is N, X₁ is N and X₂, X₃ and X₄ are each CH and X₁₅ is C. In an alternative embodiment where X₂₂ is N, X₁ and X₂ are each N and X₃, and X₄ are each CH and X₁₅ is C. In an alternative embodiment where X₂₂ is N, X₂ and X₄ are each N and X₁, and X₃ are each CH and X₁₅ is C.

In an alternative embodiment where X₂ and X₁₅ are N, X₁, X₃ and X₄ are each CH and X₂₂ is C.

In one embodiment of the compound of formula (VII), tautomer or salt thereof, R⁸, R⁹, R¹⁰, R¹¹, R¹², R¹³, R¹⁴, R¹⁵ and R³⁵ are independently selected from H or fluoro. In another embodiment, R⁸, R⁹ and R³⁵ are independently selected from H or halo and R¹⁰, R¹¹, R¹², R¹³, R¹⁴ and R¹⁵ are each H. In a more particular embodiment, R⁸ and R⁹ are independently selected from H or fluoro and R¹⁰, R¹¹, R¹², R¹³, R¹⁴, R¹⁵ and R³⁵ are each H. In a more particular embodiment, R⁸, R⁹ R¹⁰, R¹¹, R¹², R¹³, R¹⁴ and R¹⁵ are each H and R³⁵ is H or fluoro.

In one embodiment of the compound of formula (VII), tautomer a salt thereof, r is 0. In an alternative embodiment, r is 1 and R³⁶ is H. In an alternative embodiment, r is 1 and R³⁶ is methyl.

In one embodiment of the compound of formula (VII), tautomer a salt thereof, a and b are both 1 and X₁₆ is CH or N. In a more particular embodiment, a and b are both 1 and X₁₆ is N. In an alternative embodiment, a and b are both 1 and X₁₆ is CH.

In one embodiment of the compound of formula (VII), tautomer a salt thereof, a is 0 and b is 1 and X₁₆ is N.

In one embodiment of the compound of formula (VII), tautomer a salt thereof, a and b are both 0 and X₁₆ is CH.

In one embodiment of the compound of formula (VII), tautomer or salt thereof, R³⁴ is hydrogen or fluoro. In a more particular embodiment, R³⁴ is hydrogen.

In one embodiment of the compound of formula (VII), tautomer or salt thereof:
c and d are each 0; or
c is 1 and d is 0; or
c and d are each 1.

In one embodiment of the compound of formula (VII), tautomer or salt thereof, R³⁷ is H. In an alternative embodiment, R³⁷ is methyl.

In one embodiment of the compound of formula (VII), tautomer or salt thereof, R⁴² is hydrogen, methyl, fluoro or CN. In a more particular embodiment, R⁴² is hydrogen.

In one embodiment of the compound of formula (VII), tautomer or salt thereof, R³⁸, R³⁹, R⁴⁰ and R⁴¹ are independently selected from H, CF₃ and methyl. In a more particular embodiment, R³⁸, R³⁹, R⁴⁰ and R⁴¹ are independently selected from H and methyl. In a more particular embodiment, R³⁸, R³⁹, R⁴⁰ and R⁴¹ are each H.

In particular embodiment of the compound of formula (VII), tautomer or salt thereof:
X₁₀ is N and X₁₁ is CR³⁴;
s is 0;
u is 0 or 1;
v is 0, 1 or 2; and
R³⁴ is hydrogen or halo.

In more particular embodiment of the compound of formula (VII), tautomer or salt thereof where X₁₀ is N and X₁₁ is CR³⁴, s is 0, u is 1, v is 0 and R³⁴ is hydrogen or halo. In a particular embodiment, R³⁴ is H.

In more particular embodiment of of the compound of formula (VII), tautomer or salt thereof where X₁₀ is N and X₁₁ is CR³⁴, s is 0, u is 0 and v is an integer between 0 and 2 and R³⁴ is hydrogen or halo. In one embodiment, v is 0. In another embodiment, v is 1. In a further embodiment, v is 2. In a particular embodiment, R³⁴ is H.

In a different embodiment of the compound of formula (VII), tautomer or salt thereof:
X₁₀ is CH and X₁₁ is CH or N;
s is 0 or 1;
u is 0 or 1; and
v is 0 or 1.

In more particular embodiment:
X₁₀ is CH and X₁₁ is N;
s is 0 or 1; and
u and v are each 1.

In an alternative embodiment of the compound of formula (VII), tautomer or salt thereof, X₁₀ and X₁₁ are each CH and s, u and v are each 0.

In particular embodiments of the compound of formula (VII), tautomer or salt thereof, R¹⁶ is chloro or CF₃.

In particular embodiments of the compound of formula (VII), tautomer or salt thereof, R¹⁷, R¹⁸, R¹⁹ and R²⁰ are H or methyl. In one embodiment, R¹⁷ and R¹⁹ are methyl, and R¹⁸ and R²⁰ are H. In one embodiment, R¹⁷, R¹⁸, R¹⁹ and R²⁰ are each H.

In particular embodiments of the compound of formula (VII), tautomer or salt thereof, either:
X₇ and X₈ are N and X₉ is CH; or
X₈ and X₉ are N and X₇ is CH; or
X₇ and X₉ are N and X₈ is CH; or
X₇ is N and X₈ and X₉ are CH; or
X₈ is N and X₇ and X₉ are CH; or
X₇ is C—F and X₈ and X₉ are CH; or
X₇, X₈ and X₉ are each CH.

In more particular embodiments of the compound of formula (VII), tautomer or salt thereof, X₇ and X₈ are N and X₉ is CH. In particular embodiments in which X₇ and X₈ are N and X₉ is CH, R¹⁷, R¹⁸, R¹⁹ and R²⁰ are each H.

In more particular embodiments of the compound of formula (VII), tautomer or salt thereof, X₇ and X₉ are N and X₈ is CH. In particular embodiments in which X₇ and X₉ are N and X₈ is CH, R¹⁷, R¹⁸, R¹⁹ and R²⁰ are each H.

In particular embodiments, the invention provides a compound of formula (XXXXV), a tautomer of a compound of formula (XXXXV), or a salt thereof:

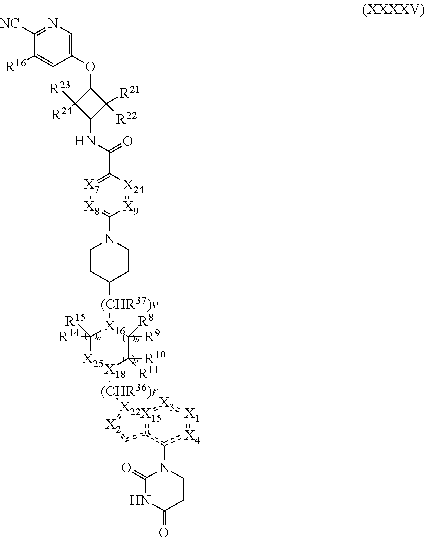

wherein:
   X₁ is N or C—R² wherein R² is selected from the group consisting of hydrogen, halogen, C₁₋₄alkyl, C₁₋₄haloalkyl, C₁₋₄alkoxy and —CONR⁵R⁶;
   X₄ is C—R⁷ or N wherein R⁷ is selected from the group consisting of hydrogen, halogen, C₁₋₃alkyl, C₁₋₃haloalkyl and C₁₋₃alkoxy;
   X₂ and X₃ are independently selected from N or CH;
   X₁₅ and X₂₂ are independently selected from N or C, wherein when X₁₅ is N, X₂₂ is C;
   r is 0, 1 or 2;
   a is 0, 1 or 2 and b and j are independently 0 or 1 with the proviso that b and j cannot both be 0;
   X₁₆ is N or CH;
   X₁₈ is CR³⁵, wherein R³⁵ is hydrogen or halogen;
   X₂₅ is CR¹²R¹³ or O;
   R⁵ and R⁶ are independently selected from hydrogen or C₁₋₄alkyl;
   R⁸, R⁹, R¹⁰, R¹¹, R¹², R¹³, R¹⁴ and R¹⁵ are independently selected from hydrogen or halogen;
   R³⁶ and R³⁷ are independently hydrogen or methyl;
   v is 0, 1 or 2;
   X₇, X₈ and X₉ are independently CH, C—F or N;
   X₂₄ is CH or N;
   R¹⁶ is halogen or CF₃; and
   R²¹, R²², R²³ and R²⁴ are independently H or C₁₋₄alkyl.

A compound of formula (XXXXV), tautomer or salt thereof comprises an androgen receptor binding moiety of formula (IIIb). The androgen receptor binding moiety of formula (IIIb) can bind to certain mutated versions of the androgen receptor including the dual mutant (T878A/L702H) Androgen Receptor. A compound of formula (XXXXV), tautomer or salt thereof is expected to be useful for the treatment of castration resistant prostate cancer.

In one embodiment of the compound of formula (XXXXV), tautomer or salt thereof, X₂₂ is N. In certain embodiments in which X₂₂ is N, one or more of X₁, X₂, X₃, X₄, are additionally N.

In one embodiment of the compound of formula (XXXXV), tautomer or salt thereof, X₁₅ is C.

In one embodiment of the compound of formula (XXXXV), tautomer or salt thereof, X₁ is N or CH.

In another embodiment of formula (XXXXV), tautomer or salt thereof, X₁ is C—R² wherein R² is selected from the group consisting of CF₃, F or Cl. In a more particular embodiment, X₁ is C—R² wherein R² is selected from the group consisting of F or Cl.

In one embodiment of the compound of formula (XXXXV), tautomer or salt thereof, X₄ is C—R⁷. In a more particular embodiment, X₄ is C—R⁷ wherein R⁷ is F, Cl or CF₃. In one embodiment, X₄ is C—R⁷ wherein R⁷ is F. In another embodiment, X₄ is CH. In an alternative embodiment, X₄ is N.

In one embodiment of the compound of formula (XXXXV), tautomer or salt thereof, either:
X₂₂ and X₂ are N and X₁, X₃ and X₄ are CH and X₁₅ is C; or
X₂₂ and X₃ are N and X₁, X₂ and X₄ are CH and X₁₅ is C; or
X₂₂ and X₄ are N and X₁, X₂ and X₃ are CH and X₁₅ is C; or
X₂₂, X₂ and X₃ are N and X and X₄ are CH and X₁₅ is C; or
X₂₂, X₃ and X₄ are N and X₁ and X₂ are CH and X₁₅ is C; or
X₂₂, X₂ and X₄ are N and X₁ and X₃ are CH and X₁₅ is C.

In one embodiment of the compound of formula (XXXXV), tautomer or salt thereof, X₁₈ is CH.

In one embodiment of the compound of formula (XXXXV), tautomer or salt thereof, r is 0 or 1. In one embodiment of the compound of formula (XXXXV), tautomer a salt thereof, r is 0. In an alternative embodiment, r is 1 and R³⁶ is H. In an alternative embodiment, r is 1 and R³⁶ is methyl.

In an alternative embodiment of the compound of formula (XXXXV), tautomer or salt thereof, r is 2 and and each R³⁶ is H.

In one embodiment of the compound of formula (XXXXV), tautomer or salt thereof, R³⁵ is H.

In one embodiment of the compound of formula (XXXXV), tautomer or salt thereof, X₁₆ is N.

In one embodiment of the compound of formula (XXXXV), tautomer or salt thereof, tautomer or salt thereof, X₂₅ is CR¹²R¹³.

In one embodiment of the compound of formula (XXXXV), tautomer or salt thereof, j is 1 and a and b are independently 0 or 1. In a more particular embodiment, X₁₆ is N, X₂₅ is CR¹²R¹³, j is 1 and a and b are independently 0 or 1. In a more particular embodiment, X₁₆ is N, X₂₅ is CR¹²R¹³, and a, b and j are each 1.

In one embodiment of the compound of formula (XXXXV), tautomer or salt thereof, a is 0 and b and j are each 1, X₁₆ is N and X₂₅ is CR¹²R¹³.

In one embodiment of the compound of formula (XXXXV), tautomer or salt thereof, R⁸, R⁹, R¹⁰, R¹¹, R¹², R¹³, R¹⁴ and R¹⁵ are independently selected from H or fluoro. In another embodiment, R⁸ and R⁹ are independently selected from H or halo and R¹⁰, R¹¹, R¹², R¹³, R¹⁴ and R¹⁵ are each H. In a more particular embodiment, R⁸ and R⁹ are independently selected from H or fluoro and R¹⁰, R¹¹, R¹², R¹³, R¹⁴ and R¹⁵ are each H. In another embodiment, R¹² and R¹³ are independently selected from H or halo and R⁸, R⁹, R¹⁰, R¹¹, R¹⁴ and R¹⁵ are each H. In a more particular embodiment, R¹² and R¹³ are independently selected from H or fluoro and R⁸, R⁹, R¹⁰, R¹¹, R¹⁴ and R¹⁵ are each H. In a more particular embodiment, R⁸, R⁹ R¹⁰, R¹¹, R¹², R¹³, R¹⁴ and R¹⁵ are each H.

In particular embodiments of the compound of formula (XXXXV), tautomer or salt thereof, v is 1.

In particular embodiments of the compound of formula (XXXXV), tautomer or salt thereof, R³⁷ is hydrogen.

In particular embodiments of the compound of formula (XXXXV), tautomer or salt thereof, R¹⁶ is chloro or CF₃. In a particular embodiment of the compound of formula (XXXXV), tautomer or salt thereof, R¹⁶ is chloro.

In particular embodiments of the compound of formula (XXXXV), tautomer or salt thereof, R²¹, R²², R²³ and R²⁴ are each methyl.

In a more particular embodiment of the compound of formula (XXXXV), tautomer or salt thereof, either:
X₇ and X₈ are N and X₉ and X₂₄ are CH; or
X₈ and X₉ are N and X₇ and X₂₄ are CH; or
X₇ is N and X₈, X₉ and X₂₄ are CH; or
X₇ and X₂₄ are N and X₈ and X₉ are CH.

In particular embodiments, the invention provides a compound of formula (XXXXVI), a tautomer of a compound of formula (XXXXVI), or a salt thereof:

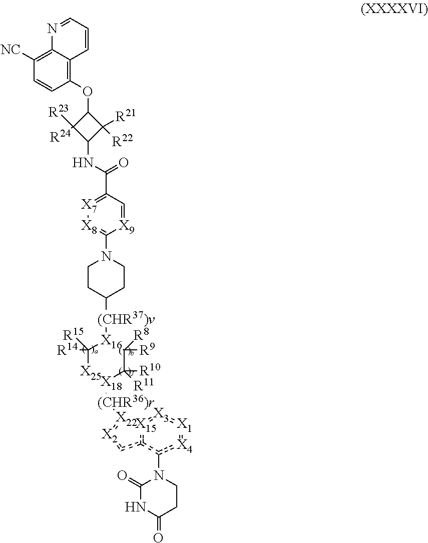

wherein:
   X₁ is N or C—R² wherein R² is selected from the group consisting of hydrogen, halogen, C₁₋₄alkyl, C₁₋₄haloalkyl, C₁₋₄alkoxy and —CONR⁵R⁶;
   X₄ is C—R⁷ or N wherein R⁷ is selected from the group consisting of hydrogen, halogen, C₁₋₃alkyl, C₁₋₃haloalkyl and C₁₋₃alkoxy;
   X₂ and X₃ are independently selected from N or CH;
   X₁₅ and X₂₂ are independently selected from N or C, wherein when X₁₅ is N, X₂₂ is C;
   r is 0, 1 or 2;
   a is 0, 1 or 2 and b and j are independently 0 or 1 with the proviso that b and j cannot both be 0;
   X₁₆ is N or CH;
   X₁₈ is CR³⁵, wherein R³⁵ is hydrogen or halogen;
   X₂₅ is CR¹²R¹³ or O;
   R⁵ and R⁶ are independently selected from hydrogen or C₁₋₄alkyl;
   R⁸, R⁹, R¹⁰, R¹¹, R¹², R¹³, R¹⁴ and R¹⁵ are independently selected from hydrogen or halogen;
   R³⁶ and R³⁷ are independently hydrogen or methyl;
   v is 0, 1 or 2;
   X₇, X₈ and X₉ are independently CH, C—F or N;
   R²¹, R²², R²³ and R²⁴ are independently H or C₁₋₄alkyl.

In one embodiment of the compound of formula (XXXXVI), tautomer or salt thereof, X₂₂ is N. In certain embodiments in which X₂₂ is N, one or more of X₁, X₂, X₃, X₄, are additionally N.

In one embodiment of the compound of formula (XXXXVI), tautomer or salt thereof, X₁₅ is C.

In one embodiment of the compound of formula (XXXXVI), tautomer or salt thereof, X₁ is N or CH.

In another embodiment of formula (XXXXVI), tautomer or salt thereof, X₁ is C—R² wherein R² is selected from the group consisting of CF₃, F or Cl. In a more particular embodiment, X₁ is C—R² wherein R² is selected from the group consisting of F or Cl.

In one embodiment of the compound of formula (XXXXVI), tautomer or salt thereof, X₄ is C—R⁷. In a more particular embodiment, X₄ is C—R⁷ wherein R⁷ is F, Cl or CF₃. In one embodiment, X₄ is C—R⁷ wherein R⁷ is F. In another embodiment, X₄ is CH. In an alternative embodiment, X₄ is N.

In one embodiment of the compound of formula (XXXXVI), tautomer or salt thereof, either:
X₂₂ and X₂ are N and X₁, X₃ and X₄ are CH and X₁₅ is C; or
X₂₂ and X₃ are N and X₁, X₂ and X₄ are CH and X₁₅ is C; or
X₂₂ and X₄ are N and X₁, X₂ and X₃ are CH and X₁₅ is C; or
X₂₂, X₂ and X₃ are N and Xand X₄ are CH and X₁₅ is C; or
X₂₂, X₃ and X₄ are N and X₁ and X₂ are CH and X₁₅ is C; or
X₂₂, X₂ and X₄ are N and X₁ and X₃ are CH and X₁₅ is C.

In one embodiment of the compound of formula (XXXXVI), tautomer or salt thereof, X₁₈ is CH.

In one embodiment of the compound of formula (XXXXVI), tautomer or salt thereof, r is 0 or 1. In one embodiment of the compound of formula (XXXXVI), tautomer a salt thereof, r is 0. In an alternative embodiment, r is 1 and R³⁶ is H. In an alternative embodiment, r is 1 and R³⁶ is methyl.

In an alternative embodiment of the compound of formula (XXXXVI), tautomer or salt thereof, r is 2 and and each R³⁶ is H.

In one embodiment of the compound of formula (XXXXVI), tautomer or salt thereof, X₁₆ is N.

In one embodiment of the compound of formula (XXXXV), tautomer or salt thereof, tautomer or salt thereof, X₂₅ is CR 12R¹³.

In one embodiment of the compound of formula (XXXXVI), tautomer or salt thereof, j is 1 and a and b are independently 0 or 1. In a more particular embodiment, X₁₆ is N, X₂₅ is CR¹²R¹³, j is 1 and a and b are independently 0 or 1. In a more particular embodiment, X₁₆ is N, X₂₅ is CR¹²R¹³ and a, b and j are each 1.

In one embodiment of the compound of formula (XXXXVI), tautomer or salt thereof, a is 0 and b and j are each 1, X₁₆ is N and X₂₅ is CR¹²R¹³.

In one embodiment of the compound of formula (XXXXVI), tautomer or salt thereof, R⁸, R⁹, R¹⁰, R¹¹, R¹², R¹³, R¹⁴ and R¹⁵ are independently selected from H or fluoro. In another embodiment, R⁸ and R⁹ are independently selected from H or halo and R¹⁰, R¹¹, R¹², R¹³, R¹⁴ and R¹⁵ are each H. In a more particular embodiment, R⁸ and R⁹ are independently selected from H or fluoro and R¹⁰, R¹¹, R¹², R¹³, R¹⁴ and R¹⁵ are each H. In another embodiment, R¹² and R¹³ are independently selected from H or halo and R⁸, R⁹, R¹⁰, R¹¹, R¹⁴ and R¹⁵ are each H. In a more particular embodiment, R¹² and R¹³ are independently selected from H or fluoro and R⁸, R⁹, R¹⁰, R¹¹, R¹⁴ and R¹⁵ are each H. In a more particular embodiment, R⁸, R⁹ R¹⁰, R¹¹, R¹², R¹³, R¹⁴ and R¹⁵ are each H.

In particular embodiments of the compound of formula (XXXXVI), tautomer or salt thereof, v is 1.

In particular embodiments of the compound of formula (XXXXVI), tautomer or salt thereof, R³⁷ is hydrogen.

In particular embodiments of the compound of formula (XXXXVI), tautomer or salt thereof, R²¹, R²², R²³ and R²⁴ are each methyl.

In particular embodiments of the compound of formula (XXXXVI), tautomer or salt thereof, either:
X₇ and X₈ are N and X₉ is CH; or
X₈ and X₉ are N and X₇ is CH; or
X₇ and X₉ are N and X₈ is CH; or
X₇ is N and X₈ and X₉ are CH; or
X₈ is N and X₇ and X₉ are CH; or
X₇ is C—F and X₈ and X₉ are CH; or
X₇, X₈ and X₉ are each CH.

In a more particular embodiment of the compound of formula (XXXXVI), tautomer or salt thereof, either:
X₇ and X₈ are N and X₉ is CH; or
X₇ is N and X₈ and X₉ are CH; or
X₈ is N and X₇ and X₉ are CH.

Compounds of formula (I), (Iaa), (Iaaa), (Ibb), (Ibbb), (Ia), (Ib), (VII), (XXXXV) and (XXXXVI) exist in tautomeric forms. The predominant form of these compounds is the lactam form (depicted in the structures). It is to be understood that any reference to a named compound or a structurally depicted compound is intended to encompass all tautomers of such compound.

In one embodiment, the invention provides a compound of formula (I), a tautomer or a pharmaceutically acceptable salt thereof that is selected from ECB1-53, a tautomer or a pharmaceutically acceptable salt thereof.

In one embodiment, the invention provides a compound of formula (I), a tautomer or a pharmaceutically acceptable salt thereof that is selected from EAR1-273, a tautomer or a pharmaceutically acceptable salt thereof.

In one embodiment, the invention provides N-((1r,4r)-4-(3-Chloro-4-cyanophenoxy)cyclohexyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide, a tautomer or a pharmaceutically acceptable salt thereof. In embodiment, the one invention provides N-((1r,4r)-4-(3-Chloro-4-cyanophenoxy)cyclohexyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide free base or a tautomer thereof. In another embodiment, the invention provides N-((1r,4r)-4-(3-Chloro-4-cyanophenoxy)cyclohexyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide hydrochloride or a tautomer thereof.

In one embodiment, the invention provides N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-indazol-1-yl)piperidin-1-yl)methyl)piperidin-1-yl)pyridazine-3-carboxamide, a tautomer or a pharmaceutically acceptable salt thereof.

In one embodiment, the invention provides N-((1r,3r)-3-(3-Chloro-4-cyanophenoxy)-2,2,4,4-tetramethylcyclobutyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-indazol-1-yl)piperidin-1-yl)methyl)piperidin-1-yl)pyridazine-3-carboxamide, a tautomer or a pharmaceutically acceptable salt thereof.

In one embodiment, the invention provides N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyridazine-3-carboxamide, a tautomer or a pharmaceutically acceptable salt thereof.

In one embodiment, the invention provides N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyridazine-3-carboxamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-2-(4-((4-(4-(2,4-dioxotetrahydropyrimidin-1(2H)-yl)-1H-indol-1-yl)piperidin-1-yl)methyl)piperidin-1-yl)pyrimidine-5-carboxamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-2-(4-((4-(4-(2,4-dioxotetrahydropyrimidin-1(2H)-yl)-1H-indazol-1-yl)piperidin-1-yl)methyl)piperidin-1-yl)pyrimidine-5-carboxamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-indazol-1-yl)piperidin-1-yl)methyl)piperidin-1-yl)nicotinamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)nicotinamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)nicotinamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)nicotinamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)nicotinamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)nicotinamide, a tautomer or a pharmaceutically acceptable salt thereof.

In one embodiment, the invention provides N-((1r,3r)-3-((8-Cyanoquinolin-5-yl)oxy)-2,2,4,4-tetramethylcyclobutyl)-5-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)picolinamide, a tautomer or a pharmaceutically acceptable salt thereof.

In one embodiment, the invention provides N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-indazol-1-yl)piperidin-1-yl)methyl)piperidin-1-yl)nicotinamide, a tautomer or a pharmaceutically acceptable salt thereof.

In one embodiment, the invention provides N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyridazine-3-carboxamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyridazine-3-carboxamide, a tautomer or a pharmaceutically acceptable salt thereof.

In one embodiment, the invention provides N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)nicotinamide, a tautomer or a pharmaceutically acceptable salt thereof.

In one embodiment, the invention provides N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)nicotinamide, a tautomer or a pharmaceutically acceptable salt thereof.

In one embodiment, the invention provides N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)nicotinamide, a tautomer or a pharmaceutically acceptable salt thereof.

In one embodiment, the invention provides N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)nicotinamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-5-(4-((4-(4-(2,4-dioxotetrahydropyrimidin-1(2H)-yl)-1H-indazol-1-yl)piperidin-1-yl)methyl)piperidin-1-yl)pyrimidine-2-carboxamide, a tautomer or a pharmaceutically acceptable salt thereof.

In one embodiment, the invention provides N-((1r,3r)-3-((5-Chloro-6-cyanopyridin-3-yl)oxy)-2,2,4,4-tetramethylcyclobutyl)-5-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyrimidine-2-carboxamide, a tautomer or a pharmaceutically acceptable salt thereof.

In one embodiment, the invention provides N-((1r,3r)-3-((6-Cyano-5-(trifluoromethyl)pyridin-3-yl)oxy)-2,2,4,4-tetramethylcyclobutyl)-5-(4-((4-(4-(2,4-dioxotetrahydropyrimidin-1(2H)-yl)-1H-indazol-1-yl)piperidin-1-yl)methyl)piperidin-1-yl)pyrimidine-2-carboxamide, a tautomer or a pharmaceutically acceptable salt thereof.

In one embodiment, the invention provides N-((1r,3r)-3-((6-Cyano-5-(trifluoromethyl)pyridin-3-yl)oxy)-2,2,4,4-tetramethylcyclobutyl)-5-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyrimidine-2-carboxamide, a tautomer or a pharmaceutically acceptable salt thereof.

In one embodiment, the invention provides N-((1r,3r)-3-(3-Chloro-4-cyanophenoxy)-2,2,4,4-tetramethylcyclobutyl)-5-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)pyrimidine-2-carboxamide, a tautomer or a pharmaceutically acceptable salt thereof.

In one embodiment, the invention provides N-((1r,3r)-3-(3-Chloro-4-cyanophenoxy)-2,2,4,4-tetramethylcyclobutyl)-5-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)pyrimidine-2-carboxamide, a tautomer or a pharmaceutically acceptable salt thereof.

In one embodiment, the invention provides, a tautomer or a pharmaceutically acceptable salt thereof.

Because of their use in medicine, salts of PROTACs including the PROTAC of formula (VII), (XXXXV) and (XXXXVI) are preferably pharmaceutically acceptable.

Pharmaceutically acceptable salts include, amongst others, those described in Berge, J. Pharm. Sci., 1977, 66, 1-19, or those listed in P H Stahl and C G Wermuth, editors, Handbook of Pharmaceutical Salts; Properties, Selection and Use, Second Edition Stahl/Wermuth: Wiley-VCH/VHCA, 2011 (see http://www.wiley.com/WileyCDA/WileyTitle/productCd-3906390519.html).

Suitable pharmaceutically acceptable salts include acid addition salts. Such acid addition salts can be formed by reaction of a compound of formula (I), (Ia), (Ib), (Ic) (VII), (XXXXV) or (XXXXVI) (which, for example contains a basic amine or other basic functional group) with the appropriate acid, optionally in a suitable solvent such as an organic solvent, to give the salt which can be isolated by a variety of methods, including crystallisation and filtration.

Salts may be prepared in situ during the final isolation and purification of a compound of formula (I), (Ia), (Ib), (Ic), (VII), (XXXXV) or (XXXXVI). If a basic compound of formula (I), (Ia), (Ib), (Ic), (VII), (XXXXV) or (XXXXVI) is isolated as a salt, the corresponding free base form of that compound may be prepared by any suitable method known to the art, including treatment of the salt with an inorganic or organic base. Similarly, if a compound of Formula (I) containing a carboxylic acid or other acidic functional group is isolated as a salt, the corresponding free acid form of that compound may be prepared by any suitable method known to the art, including treatment of the salt with an inorganic or organic acid.

Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate. It will be understood that if a compound of formula (I), (Ia), (Ib), (Ic), (VII), (XXXXV) or (XXXXVI) contains two or more basic moieties, the stoichiometry of salt formation may include 1, 2 or more equivalents of acid. Such salts would contain 1, 2 or more acid counterions, for example, a dihydrochloride salt.

Stoichiometric and non-stoichiometric forms of a pharmaceutically acceptable salt of a compound of formula (I), (Ia), (Ib), (Ic), (VII), (XXXXV) or (XXXXVI) are included within the scope of the invention, including sub-stoichiometric salts, for example where a counterion contains more than one acidic proton.

### Process for Preparing Compounds

The process to be utilized in the preparation of compounds described herein depends upon the desired compounds. Such factors as the selection of the specific substituent and various possible locations of the specific substituent all play a role in the path to be followed in the preparation of the specific compounds of this invention. Those factors are readily recognized by one of ordinary skill in the art.

In general, the compounds of the present invention may be prepared by standard techniques known in the art and by known processes analogous thereto. General methods for preparing compounds of Formula (Iaa), (Ibb), (III), (IV) and (V) are set forth below. All starting material and reagents described in the below general experimental schemes are commercially available or can be prepared by methods known to one skilled in the art. It is noted for completeness that group L is not typically synthesised as a separate intermediate. Rather, L can be synthesised attached to either the compound of formula (Iaa) or (Ibb) or the target binding moiety, or part of L can be synthesised attached to formula (Iaa) or (Ibb) and the other part of L can be synthesised attached to the target binding moiety, by standard techniques in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

General Scheme 1 provides exemplary processes of synthesis for preparing compounds of formula (Iaa) where X₁₆ is N. In General Scheme 1, X₁, X₂, X₃, X₄, X₁₅, X₁₈, X₂₂, X₂₅, R⁸, R⁹, R¹⁰, R¹¹, R¹², R¹³, R¹⁴, R¹⁵, R³⁶, a, b and r are as defined as for formula (Iaa), PG₁ and PG₂ are suitable protecting groups, L is a suitable leaving group and Hal is Br or I. Compounds of formula (XXXXI) may be prepared by methods described in Chemistry—A European Journal (2011), 17(49), 13698-13705. It is noted that where the amine version of compound (XVIII) is not readily available, this may be prepared by reduction of the nitro version of compound (XIX) with iron in the presence of ammonium chloride. The skilled person would also realise that compounds of formula (Iaa) where X₁₆ is CH could be prepared by an analogous process, without the need for protection of the nitrogen. Compounds of formula (Icc) may be prepared in an analogous manner.

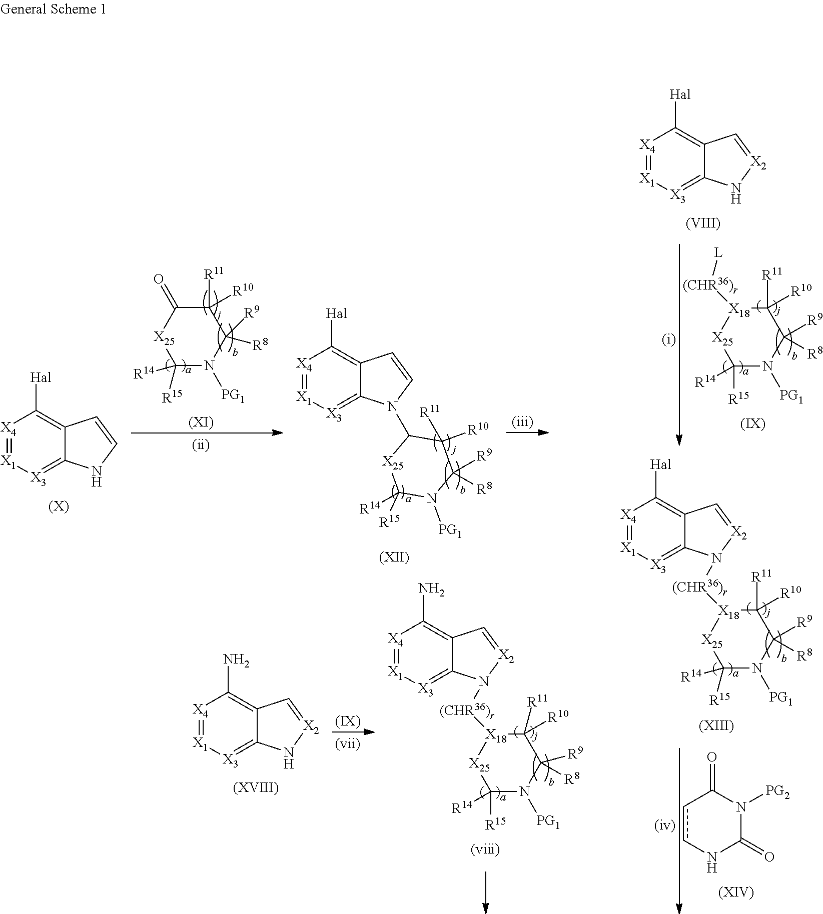

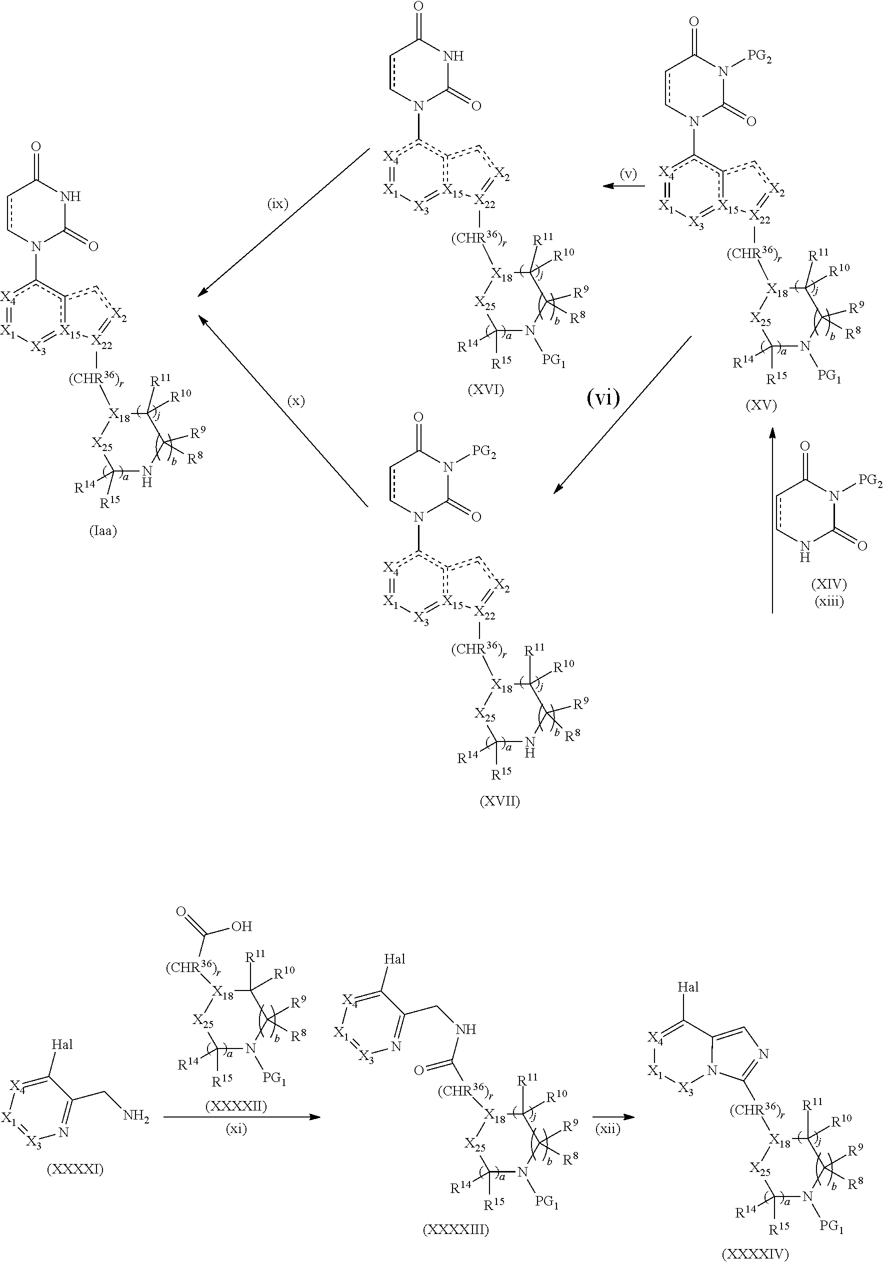

When L is bromo or methylsulfonyloxy, steps (i) and (vii) may comprise treatment with sodium hydride followed by reaction a compound of formula (IX). When L is methylsulfonyloxy, steps (i) and (vii) may alternatively comprise reaction with a compound of formula (IX) in the presence of a base such as caesium carbonate.

Step (ii) is a reductive amination reaction utilising a suitable reducing agent such as sodium triacetoxyborohydride or sodium cyanoborohydride using acetic acid. The reaction takes place at a suitable temperature such as 0° C. to room temperature.

Step (iii) is dehydrogenation reaction with a suitable oxidant such as DDQ. Step (ii) takes place in the presence of a suitable solvent such as THF at a suitable temperature such as between 0° C. and room temperature.

Steps (iv) and (xiii) are amination reactions. The reaction may be catalysed by a palladium catalyst/ligand system such as BrettPhos Pd G3 precatalyst and BrettPhos ligand in the presence of a suitable base such as tribasic potassium phosphate. Alternatively, it may be catalysed by copper (I) iodide in the presence of Trans-N,N′-Dimethylcyclohexane-1,2-diamine and a suitable base such as potassium carbonate.

Steps (v) and (x) are deprotection reactions. Where PG₂ is a trimethylsilylethoxymethyl group, deprotection may be effected by treatment with trifluoroacetic acid.

Steps (vi) and (ix) are also deprotection reactions. Where PG₁ is benzyl carboxylate, deprotection may be effected by hydrogenation catalysed by Pd—C 10% on carbon. Where PG₁ is tert-butoxycarbonyl, deprotection may be effected by treatment with trifluoroacetic acid.

Step (viii) comprises treatment of the compound of formula (IX) with acrylic acid followed by urea.

Step (xi) is an amide formation by reaction with an amine in the presence of HATU and a suitable base such as DIPEA or triethylamine.

Step (xii) is a two part reaction in which the compound of formula (XXXIII) is treated with phosphorous oxychloride, followed by di-tert-butyl dicarbonate in the presence of a suitable base such as triethylamine.

General Scheme 2 provides exemplary processes of synthesis for preparing compounds of formula (Ibbb) wherein X₁₅ is C and X₂₂ is N. In General Scheme 2, X₂, X₃, X₄, R³ and R⁴ are as defined as for formula (Ibbb) and PG₃ is a suitable protecting group. Compounds of formula (Ibbb) wherein X₁₅ is N and X₂₂ is C and compounds of formula (Ibb) can be made according to processes known to one skilled in the art.

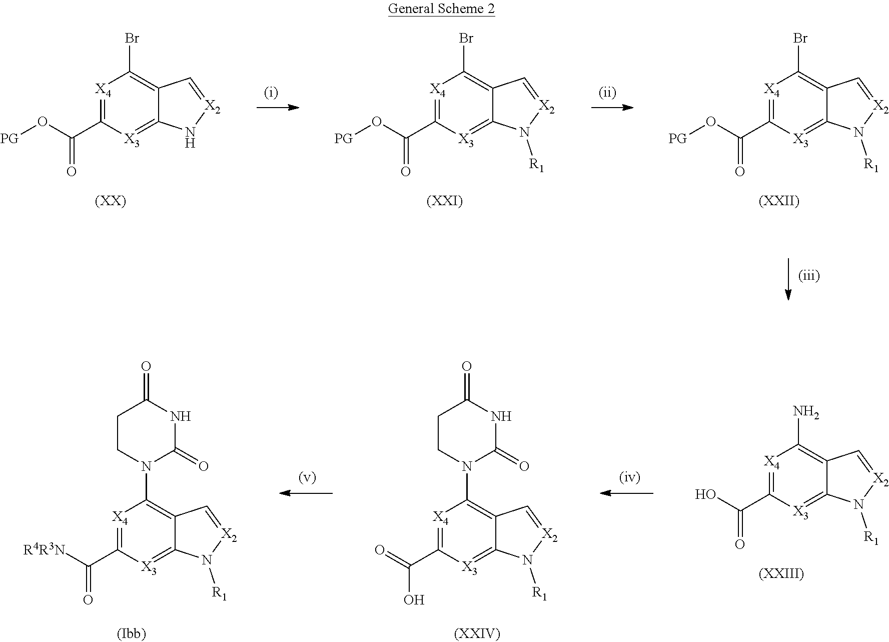

Step (i) comprises reaction with 2-iodopropane in the presence of a suitable base such as caesium carbonate.

Step (ii) comprises reaction with benzophenone imine in the presence of BINAP, Pd₂(dba)₃ and a suitable base such as cesium carbonate.

Step (iii) is a deprotection reaction, where PG₃ is a methyl group, this may comprise treatment with sodium hydroxide.

Step (iv) comprises treatment of the compound of formula (XXIII) with acrylic acid followed by urea.

Step (v) is a amide formation step by reaction with an amine in the presence of HATU and a suitable base such as triethylamine or DIPEA.

General Scheme 3 provides exemplary processes of synthesis for preparing compounds of formula (III). In General Scheme 3, A, B, X₂₃ and R¹⁶ are as defined as for formula (III) and PG₄ and PG₅ are suitable protecting groups.

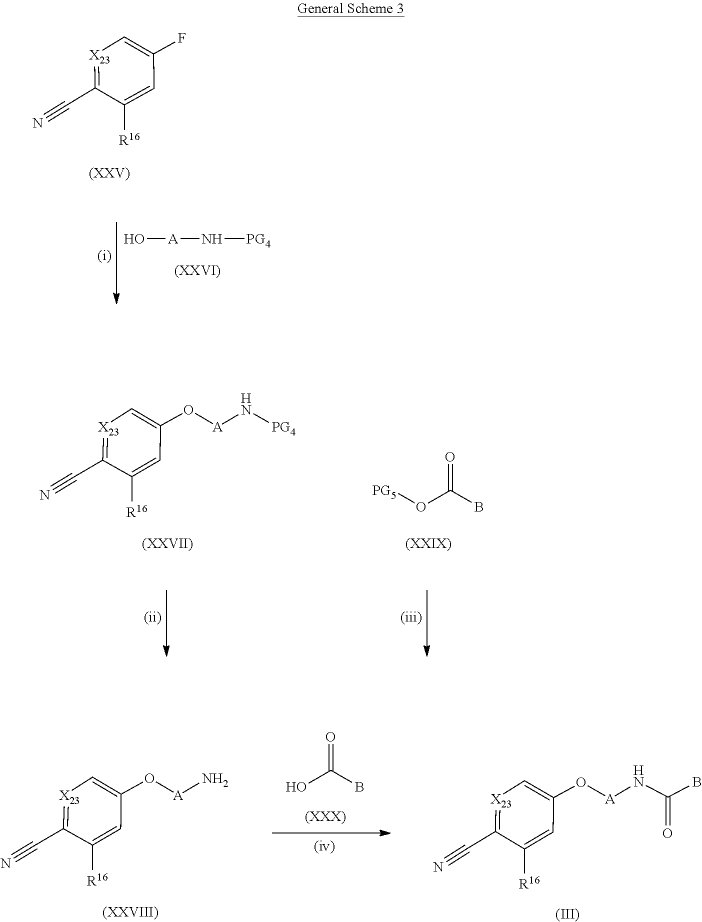

Step (i) treatment with sodium hydride followed by reaction with a compound of formula (XXVI).

Step (ii) is a deprotection reaction. When PG₄ is tert-butoxycarbonyl, deprotection may be achieved by treatment with an acid such as HCl or TFA.

Step (iii) is also a deprotection reaction. When PG₅ is methyl or ethyl, deprotection may be achieved by treatment with an alkali such as sodium hydroxide.

Step (iv) is an amide formation by reaction with an amine in the presence of HATU and a suitable base such as DIPEA or triethylamine. Alternatively, PyBOP may be used as a reagent in the presence of ethyl (E)-2-cyano-2-(hydroxyimino)acetate and N-methylmorpholine or OxymaPure and DIPEA.

General Scheme 4 provides exemplary processes of synthesis for preparing compounds of formula (IV). In General Scheme 4, R²⁵, R²⁶ and R²⁷ are as defined as for formula (IV).

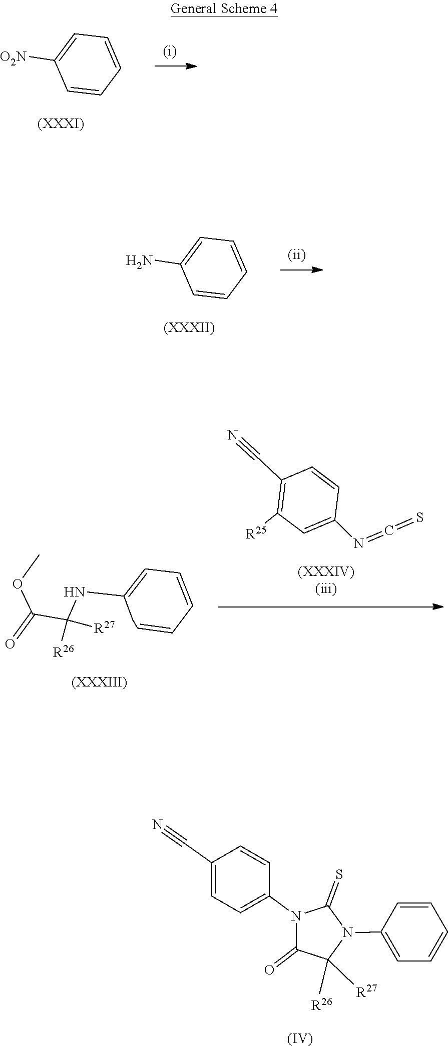

Step (i) is a hydrogenation reaction. Palladium on carbon (10 wt %) may be used as a catalyst where the reaction is performed under a hydrogen atmosphere. Alternatively, the reaction may comprise reaction with ammonium chloride and iron.

Step (ii) is an alkylation reaction. Where R²⁶ and R²⁷ are methyl, this comprises reaction with methyl 2-bromo-2-methylpropanoate in the presence of a suitable base such as DIPEA.

Step (iii) is a cyclisation reaction comprising reaction with a compound of formula (XXXIV) in the presence of a suitable solvent such as a mixture of isopropyl acetate and DMSO.

General Scheme 5 provides exemplary processes of synthesis for preparing compounds of formula (V). In General Scheme 5, R²⁹, R³⁰, R³¹, R³² and R³³ are as defined as for formula (V).

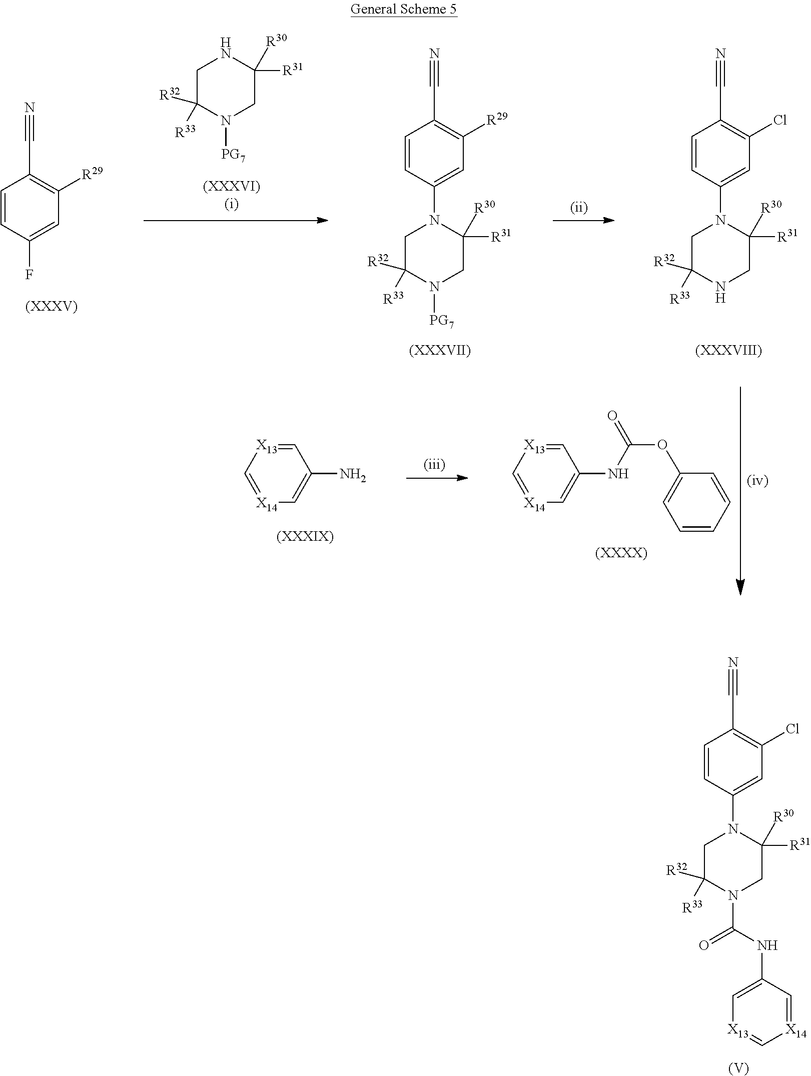

Step (i) typically takes place with the presence of a suitable base such as potassium carbonate.

Step (ii) is a deprotection reaction. When PG₇ is tert-butyl carboxylate, deprotection may be effected by treatment with acid, such as hydrochloric acid.

Step (iii) comprises reaction with phenyl carbonochloridate in the presence of a suitable base such as DIPEA.

Step (iv) takes place in a suitable solvent such as MeCN.

### Pharmaceutical Compositions

PROTACs of the invention or pharmaceutically acceptable salts thereof may be administered by any convenient route. In particular embodiments, the PROTAC or pharmaceutically acceptable salt thereof may be administered by orally, parenterally, intranasally or by inhalation. In one embodiment, the PROTAC or pharmaceutically acceptable salt thereof is administered in a pharmaceutical composition. In one embodiment, the PROTAC or pharmaceutically acceptable salt thereof is formulated in a pharmaceutical composition adapted for oral or parenteral administration, or for administration intranasally or by inhalation. Appropriate doses will readily be appreciated by those skilled in the art.

According to one aspect, the invention provides a pharmaceutical composition comprising a PROTAC or pharmaceutically acceptable salt thereof and a pharmaceutically acceptable excipient. According to another aspect, the invention provides a process for the preparation of a pharmaceutical composition comprising admixing a PROTAC or pharmaceutically acceptable salt thereof with a pharmaceutically acceptable excipient.

Pharmaceutical formulations adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Pharmaceutical formulations adapted for nasal administration can comprise a coarse powder having a particle size for example in the range 20 to 500 microns which is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the PROTAC or pharmaceutically acceptable salt thereof.

Pharmaceutical formulations adapted for administration by inhalation include fine particle dusts or mists, which may be generated by means of various types of metered, dose pressurized aerosols, nebulizers or insufflators.

Pharmaceutical formulations adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the formulations described herein may include other agents conventional in the art having regard to the type of formulation in question, for example those suitable for oral administration may include flavouring agents.

The present invention also provides unitary pharmaceutical compositions in which the PROTAC or pharmaceutically acceptable salt thereof and one or more other therapeutic agent(s) may be administered together. When a PROTAC or pharmaceutically acceptable salt thereof is used in combination with a second therapeutic agent, the dose of each therapeutic agent may differ from the dose of that therapeutic agent when used alone.

### Medical Use

Compounds of Formula (I), (Ia), (Ib), (Ic) or tautomers or pharmaceutically acceptable salts thereof wherein q is 1 may degrade the protein targeted by the TBM. Where this protein is a potential therapeutic target, compounds of Formula (I), (Ia), (Ib), (Ic) or tautomers or pharmaceutically acceptable salts thereof wherein q is 1 are useful in medicine.

Accordingly, in one aspect the invention provides a compound of formula (I), (Ia), (Ib), (Ic) or a tautomer or a pharmaceutically acceptable salt thereof wherein q is 1 for use in therapy.

Where the TBM is an androgen receptor binding moiety as described herein, compounds of formula (I), (Ia), (Ib) or (Ic), a tautomer or a pharmaceutically acceptable salt thereof wherein q is 1 are useful in the treatment of cancer, benign prostatic hyperplasia, ovarian cysts, polycystic ovary syndrome or Kennedy's Disease.

Accordingly, in one aspect, the invention provides a compound of formula (I), (Ia), (Ib), (Ic) a tautomer or a pharmaceutically acceptable salt thereof wherein q is 1 and TBM is an androgen receptor binding moiety for use in in the treatment of cancer, benign prostatic hyperplasia, ovarian cysts, polycystic ovary syndrome or Kennedy's Disease.

In another embodiment, the invention provides use of a compound of formula (I), (Ia), (Ib), or (Ic), a tautomer or a pharmaceutically acceptable salt thereof wherein q is 1 and TBM is an androgen receptor binding moiety in the manufacture of the medicament for the treatment of cancer, benign prostatic hyperplasia, ovarian cysts, polycystic ovary syndrome or Kennedy's Disease.

In another embodiment, the invention provides a method of treating cancer, benign prostatic hyperplasia, ovarian cysts, polycystic ovary syndrome or Kennedy's Disease, which method comprises administering to said subject a compound of formula (I), (Ia), (Ib) or (Ic), a tautomer or a pharmaceutically acceptable salt thereof wherein q is 1 and TBM is an androgen receptor binding moiety.

Suitably, the subject is a mammal. In a particular embodiment, the subject is human.

In one embodiment, the cancer is selected from prostate cancer, ovarian cancer, breast cancer endometrial cancer, bladder cancer, pancreatic cancer hepatocellular cancer and salivary gland cancer. In a more particular embodiment, the cancer is selected from prostate cancer or breast cancer.

In one embodiment, the prostate cancer is androgen dependent prostate cancer. In another embodiment, treatment is secondary to androgen ablation therapy. In one embodiment, treatment is secondary to treatment with abiraterone acetate or hydroxyflutamide.

In a more particular embodiment, the prostate cancer is castration resistant prostate cancer. In one embodiment, the prostate cancer is metastatic castration resistant prostate cancer. In another embodiment, the prostate cancer is non-metastatic castration resistant prostate cancer. In one embodiment, the prostate cancer is locally advanced prostate cancer.

In one embodiment, the breast cancer is triple negative breast cancer.

In one particular embodiment, the disorder treated is Kennedy's Disease.

When a compound of a compound of formula (I), (Ia), (Ib) or (Ic), a tautomer or a pharmaceutically acceptable salt thereof wherein q is 1 and TBM is an androgen receptor binding moiety is intended for use in the treatment of cancer, it may be used in combination with one or more additional anti-cancer agents, for example, a PARP inhibitor. Accordingly, in one embodiment, the invention provides a combination of a compound of formula (I), (Ia), (Ib) or (Ic), a tautomer or a pharmaceutically acceptable salt thereof wherein q is 1 and TBM is an androgen receptor binding moiety with an active pharmaceutical ingredient that is an anti-cancer agent, such as a PARP inhibitor.

### NUMBERED EMBODIMENTS: SET 1

Embodiment 1. A compound of formula (I), a tautomer of a compound of formula (I), or a salt thereof

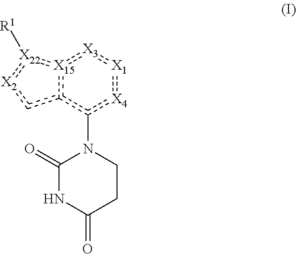

wherein:
X₁ is N or C—R² wherein R² is selected from the group consisting of hydrogen, halogen, C₁₋₄alkyl, C₁₋₄haloalkyl, C₁₋₄alkoxy, —CONR⁵R⁶ and —CONR³R⁴(L)p(TBM)q;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
n is 0 or 1;
R⁵ and R⁶ are independently selected from H or C₁₋₄alkyl;
R¹ is C₁₋₄alkyl, C₁₋₄haloalkyl, C₁₋₄alkoxy or a group of formula (II), wherein * represents the position of attachment to the compound of formula (I):

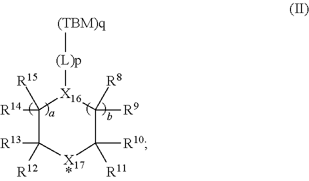

a and b are independently 0 or 1;
X₁₆ is N or CH;
X₁₇ is CR³⁴R³⁵ wherein either R³⁴ and R³⁵ together with the carbon atom to which they are attached, join together to form a cyclobutyl ring, or wherein R³⁴ is —(CHR³⁶)— or a bond to the compound of formula (I) and R³⁵ is H or a halogen;
R³⁶ is hydrogen or methyl;
R⁸, R⁹, R¹⁰, R¹¹, R¹², R¹³, R¹⁴ and R¹⁵ are independently selected from H or halogen;
L is a chemical linker;
TBM is a target binding moiety;
p and q are independently 0 and 1;
wherein when X₁₅ is N, X₂₂ is CH; and
wherein when X₁ is —CONR³R⁴(L)p(TBM)q, then R¹ is C₁₋₄alkyl, C₁₋₄haloalkyl or C₁₋₄alkoxy.
Embodiment 2. A compound of formula (I), tautomer or salt thereof according to embodiment 1, which has formula (Ia):

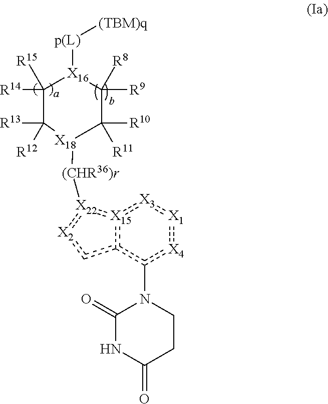

wherein:
X₁ is N or C—R² wherein R² is selected from the group consisting of hydrogen, halogen, C₁₋₄alkyl, C₁₋₄haloalkyl, C₁₋₄alkoxy and —CONR⁵R⁶;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
X₁₆ is N or CH;
X₁₈ is CR³⁵, wherein R³⁵ is H or halo;
r is 0 or 1;
R⁵ and R⁶ are independently selected from H or or C₁₋₄alkyl;
R⁸, R⁹, R¹⁰, R¹¹, R¹², R¹³, R¹⁴, R¹⁵ and R³⁵ are independently selected from H or halo; and
R³⁶ is hydrogen or methyl.
L is a chemical linker;
TBM is a target binding moiety;
p and q are independently 0 and 1; and
wherein when X₁₅ is N, X₂₂ is CH.
Embodiment 3. A compound of formula (Ia), tautomer or salt thereof according to embodiment 2, wherein X₁ is N or CH.
Embodiment 4. A compound of formula (Ia), tautomer or salt thereof according to embodiment 2 or embodiment 3, wherein X₄ is C—R⁷.
Embodiment 5. A compound of formula (Ia), tautomer or salt thereof according to embodiment 2 or embodiment 3, wherein X₄ is N.
Embodiment 6. A compound of formula (Ia), tautomer or salt thereof according to claim any one of embodiments 2 to 5, wherein X₂ is CH.
Embodiment 7. A compound of formula (Ia), tautomer or salt thereof according to any one of embodiments 2 to 6, wherein X₃ is CH.
Embodiment 8. A compound of formula (Ia), tautomer or salt thereof according to any one of embodiments 2 to 7, wherein X₁₅ is C.
Embodiment 9. A compound of formula (Ia), tautomer or salt thereof according to any one of embodiments 2 to 8, wherein X₂₂ is N.
Embodiment 10. A compound of formula (Ia), tautomer or salt thereof according to any one of embodiments 2 to 9, wherein a and b are both 1 and X₁₆ is N.
Embodiment 11. A compound of formula (Ia), tautomer or salt thereof according to any one of embodiments 2 to 10, wherein r is 0.
Embodiment 12. A compound of formula (Ia), tautomer or salt thereof according to any one of embodiments 2 to 11, wherein:
   R⁸, R⁹ and R³⁵ are independently selected from H or halo; and
   R¹⁰, R¹¹, R¹², R¹³, R¹⁴ and R¹⁵ are each H.
Embodiment 13. A compound of formula (I), tautomer or salt thereof according to embodiment 1, which has formula (Ib)

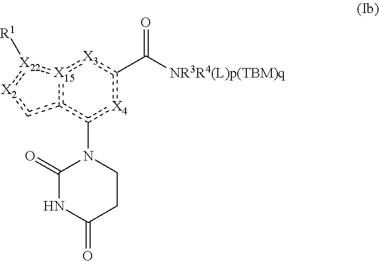

wherein:
R¹ is C₁₋₄alkyl, C₁₋₄haloalkyl or C₁₋₄alkoxy;
X₂ and X₃ are independently selected from N or CH;
X₁₅ and X₂₂ are independently selected from N or C;
X₄ is C—R⁷ or N wherein R⁷ is selected from the group consisting of hydrogen, halogen, C₁₋₃alkyl, C₁₋₃haloalkyl and C₁₋₃alkoxy;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
n is 0 or 1;
L is a chemical linker;
TBM is a target binding moiety; and
p and q are independently 0 and 1; and
wherein when X₁₅ is N, X₂₂ is CH.
Embodiment 14. A compound of formula (Ib), tautomer or salt thereof according to embodiment 13, wherein X₄ is N or CH.
Embodiment 15. A compound of formula (Ib), tautomer or salt thereof according to embodiment 14, wherein X₄ is CH.
Embodiment 16. A compound of formula (Ib), tautomer or salt thereof according to any one of embodiments 13 to 15, wherein X₂ is CH.
Embodiment 17. A compound of formula (Ib), tautomer or salt thereof according to any one of embodiments 13 to 16, wherein X₃ is CH.
Embodiment 18. A compound of formula (Ib), tautomer or salt thereof according to any one of embodiments 13 to 16, wherein X₁₅ is C.
Embodiment 19. A compound of formula (Ib), tautomer or salt thereof according to any one of embodiments 13 to 18, wherein X₂₂ is N.
Embodiment 20. A compound of formula (Ib), tautomer or salt thereof according to any one of embodiments 13 to 19, wherein R³ is H or C₁₋₄alkyl and R⁴ is —(CH₂)ₙR²⁸, wherein R²⁸ is a monocyclic nitrogen containing heterocyclic ring.
Embodiment 21. A compound of formula (Ib), tautomer or salt thereof according to any one of embodiments 13 to 19, wherein the group NR³R⁴; has a structure selected from the following:

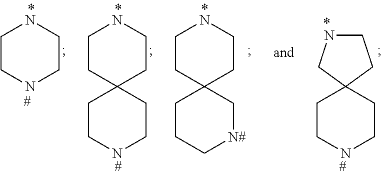

wherein the asterisk represents the position of attachment to the carbonyl group and the # represents the point of attachment to L.
Embodiment 22. A compound of formula (Ib), tautomer or salt thereof according to any one of embodiments 13 to 21, wherein R¹ is C₁₋₄alkyl.
Embodiment 23. A compound of formula (Ib), tautomer or salt thereof according to embodiment 22, wherein R¹ is isopropyl.
Embodiment 24. A compound of formula (I), (Ia), (Ib), tautomer or salt thereof according to any preceding embodiment, wherein q is 1 and the TBM is an androgen receptor binding moiety.
Embodiment 25. A compound of formula (I), (Ia), (Ib), tautomer or salt thereof according to embodiment 24, wherein the androgen receptor binding moiety has the structure of formula (III)

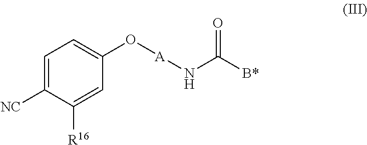

wherein the attachment position to L is shown by the asterisk;
A is selected from the group consisting of: cyclohexyl, cyclobutyl or a 6 membered nitrogen containing heterocyclic ring, wherein said cyclohexyl, cyclobutyl or 6 membered nitrogen containing heterocyclyl ring may be optionally substituted with up to 4 C₁₋₄alkyl groups;
B is selected from the group consisting of phenyl, a 6 membered nitrogen containing heteroaryl group or a fused bicyclic nitrogen containing heterocyclic ring, wherein B is optionally substituted by one or more halogen groups; and
R¹⁶ is selected from the group consisting of halo or CF₃.
Embodiment 26. A compound of formula (I), (Ia), (Ib), tautomer or salt thereof according to embodiment 25, wherein the androgen receptor binding moiety has the structure of formula (IIIa)

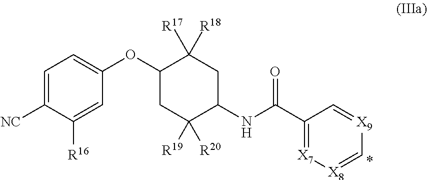

wherein the attachment position to L is shown by the asterisk;
X₇, X₈ and X₉ are independently CH, C—F or N;
R¹⁶ is selected from the group consisting of halo or CF₃;
R¹⁷, R¹⁸, R¹⁹ and R²⁰ are independently H or C₁₋₄alkyl.
Embodiment 27. A compound of formula (I), (Ia), (Ib), tautomer or salt thereof according to embodiment 26, wherein X₇ and X₈ are N and X₉ is CH.
Embodiment 28. A compound of formula (I), (Ia), (Ib), tautomer or salt thereof according to embodiment 26 or embodiment 27, wherein R¹⁷, R¹⁸, R¹⁹ and R²⁰ are each H.
Embodiment 29. A compound of formula (I), (Ia). (Ib), tautomer or salt thereof according to embodiment 25, wherein the androgen receptor binding moiety has a structure of formula (IIIb):

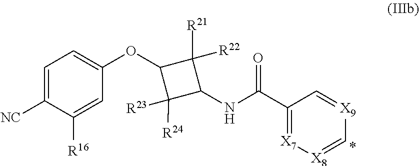

wherein the attachment position to L is shown by the asterisk;
X₇, X₈ and X₉ are independently CH or N;
R¹⁶ is selected from the group consisting of halo or CF₃; and
R²¹, R²², R²³ and R²⁴ are independently H or C₁₋₄alkyl.
Embodiment 30. A compound of formula (I), (Ia), (Ib), tautomer or salt thereof according to embodiment 29, wherein R²¹, R²², R²³ and R²⁴ are each methyl.
Embodiment 31. A compound of formula (I), (Ia), (Ib), tautomer or salt thereof according to embodiment 24, wherein the androgen receptor binding moiety has the structure of formula (IV):

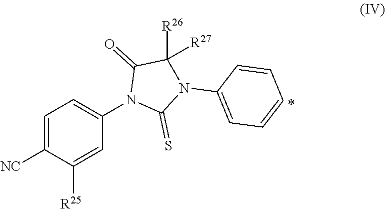

wherein the attachment position to L is shown by the asterisk;
R²⁵ is selected from the group consisting of halo or CF₃; and
R²⁶ and R²⁷ are independently H or C₁₋₄alkyl.
Embodiment 32. A compound of formula (I), (Ia), (Ib), tautomer or salt thereof according to embodiment 31, wherein R²⁶ and R²⁷ are each methyl.
Embodiment 33. A compound of formula (I), (Ia), (Ib), tautomer or salt thereof according to any one of embodiments 24 to 32, wherein p is 1 and L is a group of formula (VI):

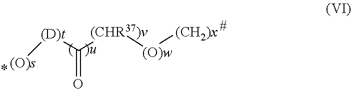

wherein * represents the attachment to the androgen receptor binding moiety, and # represents the attachment to the compound of formula (I);
D is a nitrogen containing heterocyclic ring which nitrogen containing heterocyclic ring is optionally substituted with one or more substituents selected from the group consisting of methyl, halo, CF₃ and CN;
R³⁷ is methyl or hydrogen;
s is 0 or 1;
t is 0 or 1;
u is 0 or 1;
v is 0, 1 or 2;
w is 0 or 1;
x is 0, 1, 2, 3 or 4
wherein when v is 0, u and w are not both 1;
wherein when t is 0, s and u are not both 1.
Embodiment 34. A compound of formula (I), (Ia), (Ib), tautomer or salt thereof according to embodiment 33, wherein L is a group of formula (VIa):

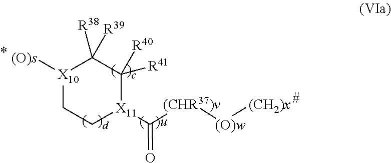

wherein * represents the attachment to the androgen receptor binding moiety, and # represents the attachment to the compound of formula (I);
X₁₀ is CH or N;
X₁₁ is a CR⁴² or N;
c is 0 or 1;
d is 0 or 1;
s is 0 or 1;
u is 0 or 1;
v is 0, 1 or 2;
w is 0 or 1;
x is 0, 1 or 2;
R³⁷ is H or methyl; and
R³⁸, R³⁹, R⁴⁰, R⁴¹ and R⁴² are independently selected from H, halo, methyl, CF₃ and CN.
Embodiment 35. A compound of formula (I), (Ia), (Ib), tautomer or salt thereof according to embodiment 34, wherein:
   X₁₀ is N and X₁₁ is CR⁴² or N;
   c is 0 or 1;
   d is 0 or 1;
   s is 0;
   u is 0 or 1; and
   v is 0, 1 or 2;
   w is 0 or 1;
   x is 0, 1 or 2; and
   R³⁷, R³⁸, R³⁹, R⁴⁰, R⁴¹ and R⁴² are independently selected from H or methyl.
Embodiment 36. A pharmaceutical composition comprising the compound, tautomer or pharmaceutically acceptable salt thereof according to any one of embodiments 24-35 and a pharmaceutically acceptable excipient.
Embodiment 37. A compound of formula (I), (Ia), (Ib), tautomer or pharmaceutically acceptable salt thereof according to any one of embodiments 24 to 35 for use in the treatment of cancer, benign prostatic hyperplasia, ovarian cysts, polycystic ovary syndrome or Kennedy's Disease.
Embodiment 38. Use of the compound, tautomer or pharmaceutically acceptable salt thereof as defined in any one of embodiments 24-35, in the manufacture of a medicament for use in the treatment of cancer, benign prostatic hyperplasia, ovarian cysts, polycystic ovary syndrome or Kennedy's Disease.
Embodiment 39. A method of treating a disorder selected from the group consisting of cancer, benign prostatic hyperplasia, ovarian cysts, polycystic ovary syndrome or Kennedy's Disease, comprising administering to a human in need thereof a therapeutically effective amount of the compound, tautomer or pharmaceutically acceptable salt thereof as defined in any one of embodiments 24-35 or the pharmaceutical composition according to embodiment 36.
Embodiment 40. A compound of formula (I), (Ia), (Ib), tautomer or salt thereof according to any one of embodiments 1 to 23, which is not 1-(1-(Piperidin-4-yl)-1H-indol-4-yl)dihydropyrimidine-2,4-(1H,3H)-dione or 1-(1-(1-Methylpiperidin-4-yl)-1H-indol-4-yl)dihydropyrimidine-2,4-(1H,3H)-dione.
Embodiment 41. A compound of formula (Ia), tautomer or salt thereof according to embodiment 9, which is not 1-(1-(piperidin-4-yl)-1H-indol-4-yl)dihydropyrimidine-2,4-(1H, 3H)-dione.

### NUMBERED EMBODIMENTS: SET 2

Embodiment 1. A compound of formula (I), a tautomer of a compound of formula (I), or a salt thereof:

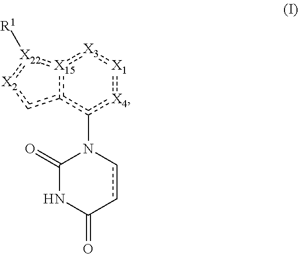

wherein:
X₁ is N or C—R² wherein R² is selected from the group consisting of hydrogen, halogen, C₁₋₄alkyl, C₁₋₄haloalkyl, C₁₋₄alkoxy, —CONR⁵R⁶ and —(CONR³R⁴)m(L)p(TBM)q;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
n is 0 or 1;
R⁵ and R⁶ are independently selected from H or C₁₋₄alkyl;
R¹ is C₁₋₄alkyl, C₁₋₄haloalkyl, C₁₋₄alkoxy or a group of formula (II), wherein * represents the position of attachment to the compound of formula (I):

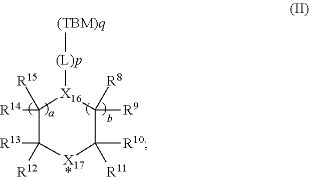

a and b are independently 0 or 1;
X₁₆ is N or CH;
X₁₇ is CR³⁴R³⁵ wherein either R³⁴ and R³⁵ together with the carbon atom to which they are attached, join together to form a cyclobutyl ring, or wherein R³⁴ is —(CHR³⁶)r-, or a bond to the compound of formula (I) and R³⁵ is H or a halogen;
r is 0, 1 or 2;
a is 0, 1 or 2 and b and j are independently 0 or 1 with the proviso that b and j cannot both be 0;
R³⁶ is hydrogen or methyl;
R⁸, R⁹, R¹⁰, R¹¹, R¹², R¹³, R¹⁴ and R¹⁵ are independently selected from H or halogen;
L is a chemical linker;
TBM is a target binding moiety;
m, p and q are independently 0 and 1;
wherein when X₁₅ is N, X₂₂ is C; and
wherein when X₁ is —(CONR³R⁴)m(L)p(TBM)q, then R¹ is C₁₋₄alkyl, C₁₋₄haloalkyl or C₁₋₄alkoxy.
Embodiment 2. A compound of formula (I), tautomer or salt thereof according to embodiment 1, which has formula (Ia):

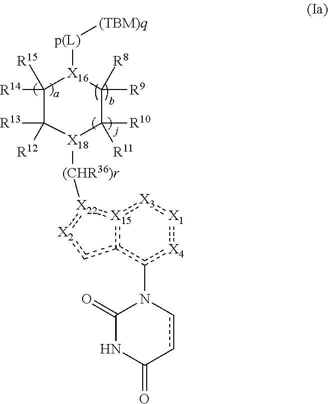

wherein:
X₁ is N or C—R² wherein R² is selected from the group consisting of hydrogen, halogen, C₁₋₄alkyl, C₁₋₄haloalkyl, C₁₋₄alkoxy and —CONR⁵R⁶;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
r is 0, 1 or 2;
a is 0, 1 or 2 and b and j are independently 0 or 1 with the proviso that b and j cannot both be 0;
X₁₆ is N or CH;
X₁₈ is CR³⁵, wherein R³⁵ is H or halo;
R⁵ and R⁶ are independently selected from H or C₁₋₄alkyl;
R⁸, R⁹, R¹⁰, R¹¹, R¹², R¹³, R¹⁴, R¹⁵ and R³⁵ are independently selected from H or halo;
R³⁶ is hydrogen or methyl;
L is a chemical linker;
TBM is a target binding moiety;
p and q are independently 0 and 1; and
wherein when X₁₅ is N, X₂₂ is CH.
Embodiment 3. A compound of formula (Ia), tautomer or salt thereof according to embodiment 2, wherein X₁ is N or CH.
Embodiment 4. A compound of formula (Ia), tautomer or salt thereof according to embodiment 2 or embodiment 3, wherein X₄ is C—R⁷.
Embodiment 5. A compound of formula (Ia), tautomer or salt thereof according to embodiment 2 or embodiment 3, wherein X₄ is N.
Embodiment 6. A compound of formula (Ia), tautomer or salt thereof according to claim any one of embodiments 2 to 5, wherein X₂ is CH.
Embodiment 7. A compound of formula (Ia), tautomer or salt thereof according to any one of embodiments 2 to 6, wherein X₃ is CH.
Embodiment 8. A compound of formula (Ia), tautomer or salt thereof according to any one of embodiments 2 to 7, wherein X₁₅ is C.
Embodiment 9. A compound of formula (Ia), tautomer or salt thereof according to any one of embodiments 2 to 8, wherein X₂₂ is N.
Embodiment 10. A compound of formula (Ia), tautomer or salt thereof according to any one of embodiments 2 to 9, wherein X₁₆ is N, j is 1 and a and b are independently 0 or 1.
Embodiment 11. A compound of formula (Ia), tautomer or salt thereof according to any one of embodiments 2 to 10, wherein r is 0.
Embodiment 12. A compound of formula (Ia), tautomer or salt thereof according to any one of embodiments 2 to 11, wherein:
   R⁸, R⁹ and R³⁵ are independently selected from H or halo; and
   R¹⁰, R¹¹, R¹², R¹³, R¹⁴ and R¹⁵ are each H.
Embodiment 13. A compound of formula (Ia), tautomer or salt thereof according to any one of embodiments 2 to 12, which has the formula (Iaaaa):

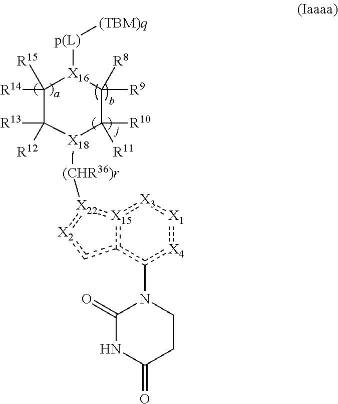

Embodiment 14. A compound of formula (I), tautomer or salt thereof according to embodiment 1, which has formula (Ib)

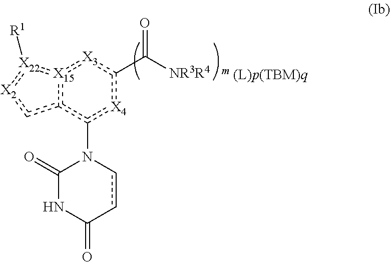

wherein:
R¹ is C₁₋₄alkyl, C₁₋₄haloalkyl or C₁₋₄alkoxy, wherein said C₁₋₄alkyl group is optionally substituted by one C₁₋₄alkoxy group;
X₂ and X₃ are independently selected from N or CH;
X₁₅ and X₂₂ are independently selected from N or C;
X₄ is C—R⁷ or N wherein R⁷ is selected from the group consisting of hydrogen, halogen, C₁₋₃alkyl, C₁₋₃haloalkyl and C₁₋₃alkoxy;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
m is 1 or 1;
n is 0 or 1;
L is a chemical linker;
TBM is a target binding moiety; and
p and q are independently 0 and 1; and
wherein when X₁₅ is N, X₂₂ is CH.
Embodiment 15. A compound of formula (Ib), tautomer or salt thereof according to embodiment 14, wherein X₄ is N or CH.
Embodiment 16. A compound of formula (Ib), tautomer or salt thereof according to embodiment 15, wherein X₄ is CH.
Embodiment 17. A compound of formula (Ib), tautomer or salt thereof according to any one of embodiments 14 to 16, wherein X₂ is CH.
Embodiment 18. A compound of formula (Ib), tautomer or salt thereof according to any one of embodiments 14 to 17, wherein X₃ is CH.
Embodiment 19. A compound of formula (Ib), tautomer or salt thereof according to any one of embodiments 14 to 18, wherein X₁₅ is C.
Embodiment 20. A compound of formula (Ib), tautomer or salt thereof according to any one of embodiments 14 to 19, wherein X₂₂ is N.
Embodiment 21. A compound of formula (Ib), tautomer or salt thereof according to any one of embodiments 14 to 20, wherein m is 1.
Embodiment 22. A compound of formula (Ib), tautomer or salt thereof according to any one of embodiments 14 to 21, wherein R³ is H or C₁₋₄alkyl and R⁴ is —(CH₂)ₙR²⁸, wherein R²⁸ is a monocyclic nitrogen containing heterocyclic ring.
Embodiment 23. A compound of formula (Ib), tautomer or salt thereof according to any one of embodiments 14 to 21, wherein the group NR³R⁴; has a structure selected from the following:

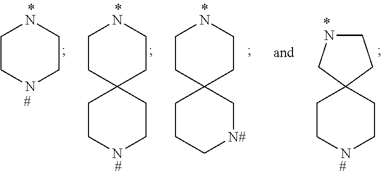

wherein the asterisk represents the position of attachment to the carbonyl group and the # represents the point of attachment to L.
Embodiment 24. A compound of formula (Ib), tautomer or salt thereof according to any one of embodiments 14 to 23, wherein R¹ is C₁₋₄alkyl.
Embodiment 25. A compound of formula (Ib), tautomer or salt thereof according to embodiment 24, wherein R¹ is isopropyl.
Embodiment 26. A compound of formula (Ib), tautomer or salt thereof according to any one of embodiments 2 to 12, which has the formula (Ibbbb):

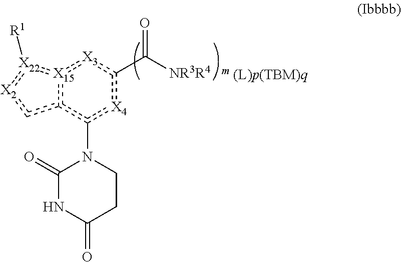

Embodiment 27. A compound of formula (I), (Ia), (Iaaaa), (Ib), (Ibbbb), tautomer or salt thereof according to any preceding embodiment, wherein q is 1 and the TBM is an androgen receptor binding moiety.
Embodiment 28. A compound of formula (I), (Ia), (Iaaaa), (Ib), (Ibbbb), tautomer or salt thereof according to embodiment 27, wherein the androgen receptor binding moiety has the structure of formula (III)

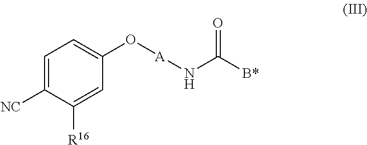

wherein the attachment position to L is shown by the asterisk;
A is selected from the group consisting of: cyclohexyl, cyclobutyl or a 6 membered nitrogen containing heterocyclic ring, wherein said cyclohexyl, cyclobutyl or 6 membered nitrogen containing heterocyclyl ring may be optionally substituted with up to 4 C₁₋₄alkyl groups;
B is selected from the group consisting of phenyl, a 6 membered nitrogen containing heteroaryl group or a fused bicyclic nitrogen containing heterocyclic ring, wherein B is optionally substituted by one or more halogen groups; and
R¹⁶ is selected from the group consisting of halo or CF₃.
Embodiment 29. A compound of formula (I), (Ia), (Iaaaa), (Ib), (Ibbbb), tautomer or salt thereof according to embodiment 28, wherein the androgen receptor binding moiety has the structure of formula (IIIa)

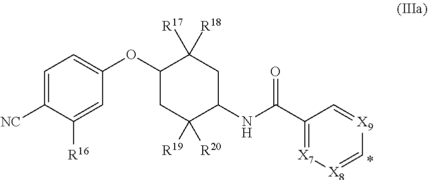

wherein the attachment position to L is shown by the asterisk;
X₇, X₈ and X₉ are independently CH, C—F or N;
R¹⁶ is selected from the group consisting of halo or CF₃;
R¹⁷, R¹⁸, R¹⁹ and R²⁰ are independently H or C₁₋₄alkyl.
Embodiment 30. A compound of formula (I), (Ia), (Iaaaa), (Ib), (Ibbbb), tautomer or salt thereof according to embodiment 29, wherein X₇ and X₈ are N and X₉ is CH.
Embodiment 31. A compound of formula (I), (Ia), (Iaaaa), (Ib), (Ibbbb), tautomer or salt thereof according to embodiment 29 or embodiment 30, wherein R¹⁷, R¹⁸, R¹⁹ and R²⁰ are each H.
Embodiment 32. A compound of formula (I), (Ia). (Iaaaa), (Ib), (Ibbbb), tautomer or salt thereof according to embodiment 28, wherein the androgen receptor binding moiety has a structure of formula (IIIb):

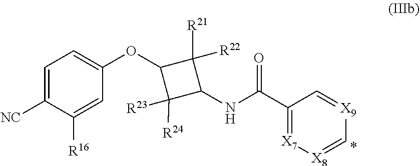

wherein the attachment position to L is shown by the asterisk;
X₇, X₈ and X₉ are independently CH or N;
R¹⁶ is selected from the group consisting of halo or CF₃; and
R²¹, R²², R²³ and R²⁴ are independently H or C₁₋₄alkyl.
Embodiment 33. A compound of formula (I), (Ia), (Iaaaa), (Ib), (Ibbbb), tautomer or salt thereof according to embodiment 32, wherein R²¹, R²², R²³ and R²⁴ are each methyl.
Embodiment 34. A compound of formula (I), (Ia), (Iaaaa), (Ib), (Ibbbb), tautomer or salt thereof according to embodiment 27, wherein the androgen receptor binding moiety has the structure of formula (IV):

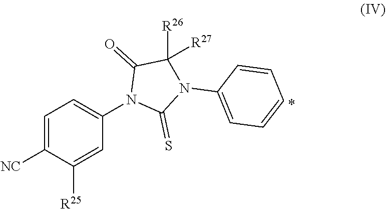

wherein the attachment position to L is shown by the asterisk;
R²⁵ is selected from the group consisting of halo or CF₃; and
R²⁶ and R²⁷ are independently H or C₁₋₄alkyl.
Embodiment 35. A compound of formula (I), (Ia), (Iaaaa), (Ib), (Ibbbb), tautomer or salt thereof according to embodiment 34, wherein R²⁶ and R²⁷ are each methyl.
Embodiment 36. A compound of formula (I), (Ia), (Iaaaa), (Ib), (Ibbbb), tautomer or salt thereof according to any one of embodiments 27 to 35, wherein p is 1 and L is a group of formula (VI):

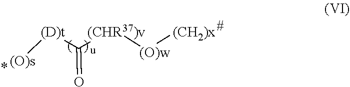

wherein * represents the attachment to the androgen receptor binding moiety, and # represents the attachment to the compound of formula (I);
D is a nitrogen containing heterocyclic ring which nitrogen containing heterocyclic ring is optionally substituted with one or more substituents selected from the group consisting of methyl, halo, CF₃ and CN;
R³⁷ is methyl or hydrogen;
s is 0 or 1;
t is 0 or 1;
u is 0 or 1;
v is 0, 1 or 2;
w is 0 or 1;
x is 0, 1, 2, 3 or 4
wherein when v is 0, u and w are not both 1;
wherein when t is 0, s and u are not both 1.
Embodiment 37. A compound of formula (I), (Ia), (Ib), tautomer or salt thereof according to embodiment 36, wherein L is a group of formula (VIa):

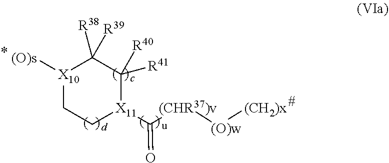

wherein * represents the attachment to the androgen receptor binding moiety, and # represents the attachment to the compound of formula (I);
X₁₀ is CH or N;
X₁₁ is a CR⁴² or N;
c is 0 or 1;
d is 0 or 1;
s is 0 or 1;
u is 0 or 1;
v is 0, 1 or 2;
w is 0 or 1;
x is 0, 1 or 2;
R³⁷ is H or methyl; and
R³⁸, R³⁹, R⁴⁰, R⁴¹ and R⁴² are independently selected from H, halo, methyl, CF₃ and CN.
Embodiment 38. A compound of formula (I), (Ia), (Ib), tautomer or salt thereof according to embodiment 37, wherein:
   X₁₀ is N and X₁₁ is CR⁴² or N;
   c is 0 or 1;
   d is 0 or 1;
   s is 0;
   u is 0 or 1; and
   v is 0, 1 or 2;
   w is 0 or 1;
   x is 0, 1 or 2; and
   R³⁷, R³⁸, R³⁹, R⁴⁰, R⁴¹ and R⁴² are independently selected from H or methyl.
Embodiment 39. N-((1r,4r)-4-(3-Chloro-4-cyanophenoxy)cyclohexyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide, a tautomer or a salt thereof.
Embodiment 40. A pharmaceutical composition comprising the compound, tautomer or pharmaceutically acceptable salt thereof according to any one of embodiments 27-39 and a pharmaceutically acceptable excipient.
Embodiment 41. A compound of formula (I), (Ia), (Ib), tautomer or pharmaceutically acceptable salt thereof according to any one of embodiments 27 to 39 for use in the treatment of cancer, benign prostatic hyperplasia, ovarian cysts, polycystic ovary syndrome or Kennedy's Disease.
Embodiment 42. Use of the compound, tautomer or pharmaceutically acceptable salt thereof as defined in any one of embodiments 27-39, in the manufacture of a medicament for use in the treatment of cancer, benign prostatic hyperplasia, ovarian cysts, polycystic ovary syndrome or Kennedy's Disease.
Embodiment 43. A method of treating a disorder selected from the group consisting of cancer, benign prostatic hyperplasia, ovarian cysts, polycystic ovary syndrome or Kennedy's Disease, comprising administering to a human in need thereof a therapeutically effective amount of the compound, tautomer or pharmaceutically acceptable salt thereof as defined in any one of embodiments 27-39 or the pharmaceutical composition according to embodiment 40.

### EXAMPLES

### Abbreviations

aq Aqueous
BINAP 2,2′-Bis(diphenylphosphino)-1,1′-binaphthalene
Boc tert-Butoxycarbonyl
BrettPhos 2-Dicyclohexylphosphino-3,6-dimethoxy-2′,4′,6′-triisopropyl-1,1′-biphenyl
DavePhos 2-Dicyclohexylphosphino-2′-(N,N-dimethylamino)biphenyl
DCE 1,2-Dichloroethane
DCM Dichloromethane
DIAD Diisopropyl azodicarboxylate
DIBAL-H Diisobutylaluminium hydride
DIPEA N,N-Diisopropylethylamine
DDQ 2,3-Dichloro-5,6-dicyano-p-benzoquinone
DMA N,N-Dimethylacetamide
DMP Dess-Martin Periodinane
DMAP 4-(Dimethylamino)pyridine
DMF N,N-Dimethylformamide
DMSO Dimethylsulfoxide
EtOAc Ethyl acetate
EtOH Ethanol
FHT Fixed hold time
GCMS Gas chromatography mass spectrometry
h Hour(s)
HATU 1-[Bis(dimethylamino)methylene]-1H-1,2,3-triazolo[4,5-b]pyridinium 3-oxid hexafluorophosphate
LCMS Liquid chromatography mass spectrometry
MDAP Mass directed autopreparative HPLC
MeCN Acetonitrile
MeOH Methanol
min Minute(s)
MsCl Methanesulfonyl chloride
MTBE tert-Butyl methyl ether
NMP N-methyl-2-pyrrolidone
NMR Nuclear Magnetic Resonance
OxymaPure Ethyl 2-cyano-2-(hydroxyimino)acetate
PCC Pyridinium chlorochromate
Pd₂(dba)₃ Tris(dibenzylideneacetone) dipalladium
PPh₃ Triphenylphosphine
PyBOP (Benzotriazol-1-yloxy)tripyrrolidinophosphonium hexafluorophosphate
Pd-PEPPSI-IPent Dichloro[1,3-bis(2,6-di-3-pentylphenyl)imidazol-2-ylidene](3-chloropyridyl)palladium(II)
Rt Retention time
RT Room temperature
TBDMS tert-Butyldimethylsilyl
TBME tert-Butyl methyl ether
TEA Trimethylamine
TFA Trifluoroacetic acid
THF Tetrahydrofuran
TMU Tetramethylurea
Tosyl-Cl 4-Toluenesulfonyl chloride
XantPhos Bis(diphenylphosphino)-9,9-dimethylxanthene

### Purification Methods

GCMS was conducted with a 30 mm×0.32 mm, 0.25 μm HP5 column using an Agilent 5977B MS detector

LCMS was conducted using one of the following methods:

### Formic Method A (“Formic A”)

LC Conditions
Column: 30 mm×2.1 mm×1.7 μm, or 50 mm×2.1 mm, 1.7 μm CSH C18
Temperature: 40° C.
Injection volume: 0.2 or 0.3 μL
Mobile phases: A=0.1% v/v solution of formic acid in water and B=0.1% v/v solution of formic acid in MeCN
Flow rate=1 mL/min

### Gradient:

| Time (min) | % A | % B |
|---|---|---|
| 0 | 97 | 3 |
| 1.5 | 3 | 97 |
| 1.9 | 3 | 97 |
| 2.0 | 98 | 2 |

The UV detection was a summed signal from wavelength of 210 nm to 350 nm.
MS Conditions
MS: Waters QDA
Ionisation mode: Alternate-scan Positive and Negative Electrospray
Scan Range: 100 to 1000 AMU
Scan Frequency: 5 hertz

### Formic Method B (“Formic B”)

LC Conditions
Column: 30 mm×2.1 mm×3.5 μm Sunfire C18
Injection volume: 0.4 μL
Mobile phases: A=0.1% formic acid in water and B=0.1% formic acid in MeCN
Gradient:

| | | Flow rate |
|---|---|---|
| Time (min) | % B | (mL/min) |
| 0 | 0 | 1.0 |
| 0.1 | 0 | 1.0 |
| 3.1 | 100 | 1.0 |
| 3.8 | 100 | 1.0 |
| 3.9 | 0 | 1.0 |
| 4.0 | 0 | 2.0 |
| 4.4 | 0 | 2.0 |
| 4.5 | 0 | 2.0 |

### High pH Method a (“High pH A”)

### LC Conditions

Column: 30 mm×2.1 mm×1.7 μm, or 50 mm×2.1 mm, 1.7 μm CSH C18
Temperature: 40° C.
Injection volume: 0.3 μL
Mobile phases: A=10 mM ammonium bicarbonate in water adjusted to pH 10 with ammonia solution
and B=MeCN
Flow rate=1 mL/min
Gradient:

| Time (min) | % A | % B |
|---|---|---|
| 0 | 100 | 0 |
| 0.05 | 100 | 0 |
| 1.5 | 3 | 97 |
| 1.9 | 3 | 97 |
| 2 | 100 | 0 |

The UV detection was a summed signal from wavelength of 210 nm to 350 nm.

### MS Conditions

MS: Waters QDA
Ionisation mode: Alternate-scan Positive and Negative Electrospray
Scan Range: 100 to 1000 AMU
Scan Frequency: 5 Hertz

### High pH Method a (“High pH B”)

### LC Conditions

Column: 50 mm×4.6 mm×3.5 μm Xbridge C8
Injection volume: 0.2 μL
Mobile phases: A=10 mM ammonium bicarbonate in water and B=MeCN
Flow rate=1.2 mL/min
Gradient:

| Time (min) | % B |
|---|---|
| 0 | 10 |
| 4.0 | 95 |
| 5.0 | 95 |
| 5.5 | 10 |
| 6.5 | 10 |

### TFA Method A (“TFA A”)

Column: 30 mm×2.1 mm, 1.7 μm CSH C18
Temperature: 45° C.
Injection volume: 0.5 μL
Mobile phases: A=0.1% v/v solution of TFA in water and B=0.1% v/v solution of TFA in MeCN
Flow rate=1.3 mL/min
Gradient:

| Time (min) | % A | % B |
|---|---|---|
| 0 | 99 | 1 |
| 1.85 | 0 | 100 |
| 1.9 | 0 | 100 |
| 2.0 | 99 | 1 |

UV detection was an averaged signal from wavelength of 210 nm to 350 nm.

### MS Conditions (Waters SQD or QDa)

### QDa Settings:

MS: Waters Acquity QDa mass detector
Ionisation mode: Alternate-scan Positive and Negative Electrospray
Scan Range: 100 to 1000 AMU
Targeted Sampling Frequency: 8 Hz

### SQD Settings:

MS: Waters Acquity SQD
Ionisation mode: Alternate-scan Positive and Negative Electrospray
Scan Range: 100 to 1000 AMU
Scan Time: 0.1 seconds

### TFA Method B (“TFA B”)

Column: 50 mm×4.6 mm, 5.0 μm Atlantis C18
Injection volume: 2.0 μL
Mobile phases: A=0.1% v/v solution of TFA in water and B=MeOH
Flow rate=1.0 mL/min
Gradient:

| Time (min) | % B |
|---|---|
| 0 | 5 |
| 5 | 95 |
| 6.5 | 95 |
| 6.8 | 5 |
| 8.0 | 5 |

Mass-directed autopurification (MDAP) was conducted using any of these methods:

### MDAP Formic

Column: 150 mm×30 mm, 5 μm or 75 mm×30 mm, 5 μm XSelect CSH C18
Mobile Phase A: 0.1% v/v solution of formic acid in Water
Mobile Phase B: 0.1% v/v solution of formic acid in MeCN.
   Total Flow Rate: 40 mL/min
   Temperature: Ambient
   Injection Volume: varied
   Instrument Name: Waters MDAP
   UV Detection Parameters: 210-350 nm
   Gradient:

Gradients ranged from 100% A and 0% B to 0% A and 100% B across various lengths of time up to 32 min.

### MDAP High pH

Column: 150 mm×30 mm, 5 μm or 100 mm×19 mm, 5 μm or 75 mm×30 mm, 5 μm XSelect CSH C18
Mobile Phase A: 10 mM ammonium bicarbonate in water adjusted to pH 10 with ammonia solution
Mobile Phase B: MeCN
Total Flow Rate: 40 mL/min
Temperature: Ambient
Injection Volume: varied
Instrument Name: Waters MDAP
UV Detection Parameters: 210-350 nm (1.2 nm Resolution; 1 Hz)
Gradient:

Gradients ranged from 100% A and 0% B to 0% A and 100% B across various lengths of time up to 32 min.

### MDAP TFA

Column: 150 mm×30 mm, 5 μm Sunfire C18
Mobile Phase A: 0.1% TFA in water
Mobile Phase B: MeOH
Total Flow Rate: 40 ml/min
Temperature: Ambient
Injection Volume: varied volume, typically 300-350 μL
Instrument Name: Waters MDAP
UV Detection Parameters: 210-350 nm (1.2 nm Resolution; 1 Hz)
MS Detection Parameters: Waters QDa; ESI+/−; 100-1000AMU; 0.3 s/scan; 0.1 s inter-scan delay
Collection Mode: Fractionation by UV (DAD) and MS(+/−)

The gradient was delivered by two pumps in order to use at-column dilution when injecting the sample. The gradient program used for the purification is summarised below:

| Chromatographic Pump: | | | Regeneration Pump: | | |
|---|---|---|---|---|---|
| Time | | Flow Rate | Time | | Flow Rate |
| (min) | % B | (mL/min) | (min) | % B | (mL/min) |
| 0 | 35 | 4 | 0 | 25 | 36 |
| 4 | 35 | 4 | 4 | 25 | 36 |
| 4.5 | 35 | 20 | 4.5 | 35 | 20 |
| 16 | 70 | 20 | 16 | 70 | 20 |
| 16.5 | 100 | 20 | 16.5 | 100 | 20 |
| 19 | 100 | 20 | 19 | 100 | 20 |

### SFC1

Column: 150 mm×30 mm, 5 μm, Reprospher PEI
Mobile Phase A: CO₂
Mobile Phase B: 0.5% v/v isopropylamine in MeOH
Total Flow Rate: 120 mL/min
Temperature: 55° C.
Injection Volume: 2000 μL injected every 6.5 min
Instrument: PIC Solutions ‘Sapphire (PIC-1)’ Preparative SFC
Outlet Pressure: 130 bar
UV Detection Parameters: 220 nm
MS Detection Parameters: None
Collection Mode: Fractionation by Time and UV (DAD)

| Time (mins) | % B |
|---|---|
| 0 | 35 |
| 0.25 | 35 |
| 3.75 | 50 |
| 4.1 | 35 |
| 4.5 | 35 |
| 5.5 | 35 |

### SFC2

Column: 150 mm×30 mm, 5 μm, Reprospher PEI
Mobile Phase A: CO₂
Mobile Phase B: 0.5% v/v Isopropylamine in MeOH
Total Flow Rate: 120 mL/min
Temperature: 55° C.
Injection Volume: 400 μL injected every 6.5 mins
Instrument: PIC Solutions ‘Sapphire (PIC-1)’ Preparative SFC
Outlet Pressure: 130 bar
UV Detection Parameters: 220 nm
MS Detection Parameters: None
Collection Mode: Fractionation by Time and UV (DAD)

| Time (min) | % B |
|---|---|
| 0 | 35 |
| 0.25 | 35 |
| 3.75 | 50 |
| 4.1 | 55 |
| 5.49 | 55 |
| 5.5 | 35 |
| 5.9 | 35 |

Appropriate fractions were combined then evaporated to dryness using a rotary evaporator. The dried sample was then transferred in a low volume of MeOH into a final vial. The sample was then blown down under a nitrogen flow at 25° C. to dryness to yield the title compound

### INTERMEDIATES

### Description 1

### 3-((2-(Trimethylsilyl)ethoxy)methyl)dihydropyrimidine-2,4(1H,3H)-dione (D1)

Dihydropyrimidine-2,4(1H,3H)-dione (50 g×2, 438 mmol) was suspended in DMF (500 mL) and heated to 140° C. until the solution was clear. The reaction mixture was cooled to room temperature and added Cs₂CO₃ (214 g, 657 mmol) portion wise over a period of 30 min. The reaction mixture was further cooled to 10° C. and added (2-(chloromethoxy)ethyl)trimethylsilane (29.2 g, 175 mmol) dropwise and then reaction was allowed to stir at room temperature for 48 h. Reaction mixture was filtered under vacuum. Filtrate was diluted with water (2 L) and extracted with EtOAc (2×1 L). The combined organic layers were evaporated under reduced pressure to give crude compound. Crude was purified portion wise, dissolved in DCM (200 mL) and purified using a 330 g silica column, eluting with 50-100% EtOAc:petroleum ether, and the desired fractions were combined and concentrated in vacuo to give the title compound as a colourless gummy solid (47 g, 187 mmol, 21% yield). GCMS: Rt=4.94 min, M−H⁻=243.1

### Description 2

### Benzyl 4-(4-bromoindolin-1-yl)piperidine-1-carboxylate (D2)

Benzyl 4-oxopiperidine-1-carboxylate (8 g, 34.3 mmol) and 4-bromoindoline (6 g, 30.3 mmol) were dissolved in acetic acid (100 mL) and stirred for 1 h, then cooled in an ice bath and sodium triacetoxyborohydride (12.84 g, 60.6 mmol) was added. The mixture was stirred for 18 h, then evaporated to about half its original volume, diluted with water (200 mL) and extracted with EtOAc (2×200 mL). The combined organics were washed with water and sodium bicarbonate solution (200 ml of each) and then dried and evaporated in vacuo to give the title compound as a pale yellow gum (16.0 g) which was used in the next step without purification. LCMS (high pH A): Rt=1.52 min, MH⁺=415.1, 417.1

### Description 3

### Benzyl 4-(4-bromo-1H-indol-1-yl)piperidine-1-carboxylate (D3)

Benzyl 4-(4-bromoindolin-1-yl)piperidine-1-carboxylate (may be prepared as described in Description 2; 20 g, 38.5 mmol) (crude, assumed to be about 80% pure) was dissolved in THF (60 mL) and cooled in an ice bath, then DDQ (8.74 g, 38.5 mmol) was added and the mixture stirred for 10 min, then allowed to warm to room temperature. The mixture was diluted with EtOAc (200 mL) and washed with sodium bicarbonate solution (200 mL), then with 1 M NaOH (200 mL) and the organic layer dried and evaporated in vacuo to give a dark brown gum. The crude product was dissolved in DCM and loaded onto a 330 g silica column, then eluted with 0-50% MTBE/cyclohexane and product-containing fractions evaporated in vacuo to give the title compound (15.7 g, 38.0 mmol, 99% yield) as a pale yellow gum. LCMS (high pH A): Rt=1.52 min, MH⁺=413.1, 415.1.

### Description 4

### Benzyl 4-(4-(2,4-dioxo-3-((2-(trimethylsilyl)ethoxy)methyl)tetrahydropyrimidin-1(2H)-yl)-1H-indol-1-yl)piperidine-1-carboxylate (D4)

To BrettPhos Pd G3 (1.162 g, 1.282 mmol), BrettPhos (0.688 g, 1.282 mmol)₃-((2-(trimethylsilyl)ethoxy)methyl)dihydropyrimidine-2,4(1H,3H)-dione (may be prepared as described in Description 1; 4 g, 16.37 mmol) and potassium phosphate tribasic (6.80 g, 32.1 mmol) was added a solution of benzyl 4-(4-bromo-1H-indol-1-yl)piperidine-1-carboxylate (may be prepared as described in Description 3; 5.3 g, 12.82 mmol) in 1,4-dioxane (100 mL). The reaction mixture was degassed (vacuum/nitrogen×3) and then heated under nitrogen to 100° C. for 18 h. The reaction mixture was allowed to cool. The mixture was diluted with EtOAc and filtered through a pad of celite. The pad was washed with EtOAc. The combined filtrate and washings were evaporated in vacuo. The residue was dissolved in DCM and applied to a 330 g silica cartridge. This was eluted with a gradient of 0-60% EtOAc in cyclohexane over 30 min. The required fractions were combined and evaporated in vacuo to give the title compound (5.61 g, 9.73 mmol, 76% yield). LCMS (high pH A): Rt=1.44 min, MH⁺=577.1

### Description 5

### Benzyl 4-(4-(2,4-dioxotetrahydropyrimidin-1(2H)-yl)-1H-indol-1-yl)piperidine-1-carboxylate (D5)

Benzyl 4-(4-(2,4-dioxo-3-((2-(trimethylsilyl)ethoxy)methyl)tetrahydropyrimidin-1(2H)-yl)-1H-indol-1-yl)piperidine-1-carboxylate (may be prepared as described in Description 4; 7.6 g, 13.18 mmol) was dissolved in DCM (30 mL) and cooled to 0° C. in an ice bath, then TFA (10 mL, 130 mmol) was added and the mixture was stirred for 2 h, then evaporated in vacuo to give a brown oil. This was suspended in MeOH (30 mL) and treated with SG0.88 ammonium hydroxide (20 mL) and stirred for 10 min, then diluted with DCM (100 mL) and washed with saturated sodium bicarbonate solution. The organic layer was dried and evaporated in vacuo to give a beige solid. The crude was dissolved in DCM and loaded onto a 120 g silica column, then eluted with 0-100% EtOAc/cyclohexane and product-containing fractions evaporated in vacuo to give the title compound as a colourless solid (5.23 g, 11.71 mmol, 89% yield). LCMS (high pH A): Rt=1.07 min, MH⁺=447.2.

### Description 6

### Tert-butyl 4-((4-bromo-1H-indol-1-yl)methyl)piperidine-1-carboxylate (D6)

To a solution of 4-bromo-1H-indole (0.3 mL, 2.392 mmol) in DMF (13 mL) stirred under nitrogen at 0° C. was added 60% sodium hydride (0.124 g, 3.11 mmol), portionwise. The reaction mixture was stirred at 0° C. for 5 min. To the reaction mixture was added was added tert-butyl 4-(bromomethyl)piperidine-1-carboxylate (0.8 g, 2.88 mmol) and potassium iodide (0.476 g, 2.87 mmol). The reaction mixture was stirred at 60° C. for 1.5 h, then cooled to room temperature. The reaction was quenched by addition of saturated aqueous ammonium chloride (50 mL), and the mixture was extracted with EtOAc (3×50 mL). The organics were combined, washed with 5% aq. LiCl (4×25 mL), brine (40 mL), dried by passing through a hydrophobic frit and the solvent removed in vacuo. The residue was dissolved in DCM (1 mL) and loaded onto a cyclohexane preconditioned 80 g Redisep silica column. The crude material on silica was eluted by Combiflash with 100% EtOAc (user error). The fractions containing the crude product were collected and the solvent removed in vacuo. The resulting crude gum was dissolved in DCM (1 mL) and loaded onto a cyclohexane preconditioned 80 g Redisep silica column. The crude material on silica was purified by Combiflash using a gradient of 0-20% TBME in cyclohexane over 12 column volumes, followed by 20-30% TBME in cyclohexane over 3 column volumes. Fractions containing the desired product were collected, and the solvent was removed in vacuo to give a pale yellow gum (Contains 5% cyclohexane and 8% TBME by NMR) The sample was further dried in the vacuum oven to give the title compound as a pale yellow solid (560 mg, 1.424 mmol, 60% yield). LCMS (high pH A): Rt=1.52 min, MH⁺=337, 339.

### Description 7

### tert-Butyl 4-((4-(2,4-dioxo-3-((2-(trimethylsilyl)ethoxy)methyl)tetrahydropyrimidin-1(2H)-yl)-1H-indol-1-yl)methyl)piperidine-1-carboxylate (D7)

3-((2-(Trimethylsilyl)ethoxy)methyl)dihydropyrimidine-2,4(1H,3H)-dione (maybe prepared as described in Description 1; 383 mg, 1.566 mmol), potassium carbonate (394 mg, 2.85 mmol), copper(I) iodide (40.7 mg, 0.214 mmol), Trans-N,N′-Dimethylcyclohexane-1,2-diamine (0.067 mL, 0.427 mmol) and a solution of tert-butyl 4-((4-bromo-1H-indol-1-yl)methyl)piperidine-1-carboxylate (may be prepared as described in Description 6; 560 mg, 1.424 mmol) in anhydrous 1,4-dioxane (16.4 mL) were mixed in a microwave vial, flushed with nitrogen, sealed and heated at 140° C. for 16 h. The reaction mixture was evaporated in vacuo, diluted with DCM (25 mL) and water (25 mL). The layers were separated, and the aqueous was extracted with DCM (25 mL). The organics were combined, dried by passing through a hydrophobic frit and concentrated in vacuo. The crude material was dissolved in minimal DCM and loaded onto a cyclohexane preconditioned 40 g Redisep silica column and purified by Combiflash using a gradient of 0-60% EtOAc in cyclohexane over 15 column volumes. Fractions containing the desired product were collected and the solvent removed in vacuo and under high vacuum to give the title compound as a light brown foam (528 mg, 0.948 mmol, 67% yield). LCMS (high pH A): Rt=1.47 min, MNH₄⁺=574.

### Description 8

### Tert-butyl 4-(4-bromo-1H-indol-1-yl)-3,3-difluoropiperidine-1-carboxylate (D8)

4-Bromoindoline (1 g, 5.05 mmol) and tert-butyl 3,3-difluoro-4-oxopiperidine-1-carboxylate (1.425 g, 6.06 mmol) were heated at 140° C. for 40 min, then cooled to room temperature. Acetic acid (10 mL) and sodium cyanoborohydride (0.952 g, 15.15 mmol) were added, and the reaction was stirred for 5 h, then overnight. The reaction was diluted with ether and 1 M NaOH solution. The organic phase was washed with brine (twice), dried (MgSO4), filtered and evaporated in vacuo. The residue was taken up in THF (10.00 mL), cooled to 0° C., treated with DDQ (1.719 g, 7.57 mmol) and allowed to warm to room temperature. After 1 h, the reaction mixture was diluted with EtOAc and washed with NaOH solution, sodium bicarbonate solution and brine, dried (MgSO4), filtered and evaporated in vacuo to give a brown oil. The material was purified by flash chromatography (silica, 120 g, 0-30% EtOAc/cyclohexane) to give the title compound as a pale yellow collapsed foam (1.35 g, 3.09 mmol, 61% yield). LCMS (high pH A): Rt=1.41 min, MH⁺=415, 417.

### Description 9

### Benzyl 4-(4-bromo-6-fluoro-1H-indol-1-yl)piperidine-1-carboxylate (D9)

A mixture of 4-bromo-6-fluoro-1H-indole (917 mg, 4.28 mmol), benzyl 4-((methylsulfonyl)oxy)piperidine-1-carboxylate (2014 mg, 6.43 mmol) and cesium carbonate (2094 mg, 6.43 mmol) in anhydrous DMF (11 mL), was stirred at 100° C. for 16 h. The reaction mixture was allowed to cool to room temperature and diluted with EtOAc (50 mL). The solution was washed sequentially with water (50 mL), 5% LiCl (aq) (50 mL) and brine (50 mL). The organic layer was passed through a hydrophobic frit and the filtrate evaporated in vacuo. The oil (ca 1.8 g) was purified by reverse phase flash chromatography on a 100 g C18 cartridge eluting with a 30-95% gradient of MeCN and 10 mM ammonium carbonate in water adjusted to pH10 with ammonia solution over 22 min using a 60 mL/min flow rate. The appropriate fractions were combined and the solvent removed by rotary evaporation to give the title compound as a dark brown gum (267 mg, 0.619 mmol, 14% yield). LCMS (high pH A): Rt=1.47 min, MH⁺=431, 433.

### Description 10

### Benzyl 4-(4-(2,4-dioxo-3-((2-(trimethylsilyl)ethoxy)methyl)tetrahydropyrimidin-1(2H)-yl)-6-fluoro-1H-indol-1-yl)piperidine-1-carboxylate (D10)

A mixture of copper(I) iodide (12 mg, 0.063 mmol), potassium carbonate (170 mg, 1.228 mmol) and 3-((2-(trimethylsilyl)ethoxy)methyl)dihydropyrimidine-2,4(1H,3H)-dione (maybe prepared as described in Description 1; 150 mg, 0.614 mmol) was diluted with a solution of benzyl 4-(4-bromo-6-fluoro-1H-indol-1-yl)piperidine-1-carboxylate (may be prepared as described in Description 9; 264 mg, 0.612 mmol) in anhydrous 1,4-dioxane (3.0 mL). Trans-N,N′-Dimethylcyclohexane-1,2-diamine (0.019 mL, 0.123 mmol) was added, the vessel sealed, and evacuated and purged with nitrogen (×3). The mixture was stirred at 140° C. for 4 h. The reaction was allowed to cool to room temperature, the lid removed, and further copper(I) iodide (24 mg) and Trans-N,N′-Dimethylcyclohexane-1,2-diamine (0.019 mL, 0.123 mmol) added. The vial was sealed and evacuated and purged with nitrogen (×3). The mixture was stirred at 140° C. for 16 h. The reaction was allowed to cool to room temperature, the suspension diluted with DCM (5 mL), and passed through a hydrophobic frit. The filtrate was evaporated in vacuo and the residue loaded in DCM (3 mL) and purified on a 24 g silica cartridge using a gradient of 0-75% EtOAc in cyclohexane over 14 column volumes. The appropriate fractions were combined and the solvent evaporated in vacuo to give the title compound as a light brown gum (270 mg, 0.454 mmol, 74% yield). LCMS (high pH A): Rt=1.42 min, M-C₂H₆+567.

### Description 11

### 1-(6-Fluoro-1-(piperidin-4-yl)-1H-indol-4-yl)-3-((2-(trimethylsilyl)ethoxy)methyl)dihydropyrimidine-2,4(1H,3H)-dione (D11)

A flask containing 10 wt. % palladium on carbon (22 mg, 0.021 mmol) was evacuated and purged with nitrogen (×3) and a solution of benzyl 4-(4-(2,4-dioxo-3-((2-(trimethylsilyl)ethoxy)methyl)tetrahydropyrimidin-1(2H)-yl)-6-fluoro-1H-indol-1-yl)piperidine-1-carboxylate (may be prepared as described in Description 10; 235 mg, 0.395 mmol) in Ethanol (20 mL) added. The mixture was evacuated and purged with nitrogen (×3) and stirred under atmospheric pressure hydrogen at room temperature for 16 h. The mixture was evacuated and purged with nitrogen (×3) and 10 wt. % palladium on carbon (22 mg, 0.021 mmol) added. The mixture was evacuated and purged with nitrogen (×3) and stirred under atmospheric pressure hydrogen at room temperature for 24 h. The reaction mixture was filtered through Celite and the pad washed with ethanol (20 mL). The filtrate was evaporated in vacuo and the gum dried under high vacuum to give the title compound as a light green gum (155 mg, 0.336 mmol). LCMS (high pH A): Rt=1.22 min, MH⁺=461.

### Description 12

### Tert-butyl 4-(4-bromo-1H-indazol-1-yl)piperidine-1-carboxylate (D12)

4-bromo-1H-indazole (38 g, 193 mmol) was dissolved in DMF (400 mL). Cs₂CO₃ (126 g, 386 mmol) and tert-butyl 4-((methylsulfonyl)oxy)piperidine-1-carboxylate (81 g, 289 mmol) were added, and the reaction mixture heated at 60° C. for 16 h. The reaction mixture was quenched by adding water (500 mL) and extracted with EtOAc (2×500 mL). The combined organic layers were washed with water (250 mL), dried over anhydrous sodium sulphate (3 g) and concentrated under reduced pressure. The crude material was purified on a 330 g silica cartridge using EtOAc in hexane as eluent to give the title compound as an orange gummy solid (29 g, 76 mmol, 40% yield). LCMS (formic A): Rt=1.33 min, M-^{t}Bu⁺=324.

### Description 13

### Tert-butyl 4-(4-(2,4-dioxo-3-((2-(trimethylsilyl)ethoxy)methyl)tetrahydropyrimidin-1(2H)-yl)-1H-indazol-1-yl)piperidine-1-carboxylate (D13)

To a solution of tert-butyl 4-(4-bromo-1H-indazol-1-yl)piperidine-1-carboxylate (may be prepared as described in Description 12; 1 g, 2.63 mmol), 3-((2-(trimethylsilyl)ethoxy)methyl)dihydropyrimidine-2,4(1H,3H)-dione (may be prepared as described in Description 1; 0.643 g, 2.63 mmol), (1R,2R)—N1,N2-dimethylcyclohexane-1,2-diamine (0.224 g, 1.578 mmol) and K₂CO₃ (0.909 g, 6.57 mmol) in 1,4-dioxane (20 mL) was purged with nitrogen gas for 5 min. Copper(I) iodide (0.100 g, 0.526 mmol) was added and the mixture purged with nitrogen gas for 5 min. The reaction was stirred at 120° C. in a microwave for 3 h. This reaction procedure was repeated for a further 5× batches. The 6× reaction mixtures were filtered through Celite and the filtrate concentrated under reduced pressure. The material was washed with water (100 mL) and dried under vacuum. The material was dissolved in DCM (20 mL) and loaded onto a 120 g silica cartridge and purified using 40% EtOAc in hexane to give the title compound as an orange gummy liquid (4.7 g, 8.46 mmol, 54% yield). LCMS (formic A): Rt=1.31 min, M-C₆H₁₁⁺=460.

### Description 14

### Tert-butyl 4-(6-chloro-4-iodo-1H-indazol-1-yl)piperidine-1-carboxylate (D14)

A mixture of 6-chloro-4-iodo-1H-indazole (216 mg, 0.776 mmol), tert-butyl 4-((methylsulfonyl)oxy)piperidine-1-carboxylate (260 mg, 0.931 mmol) and cesium carbonate (379 mg, 1.163 mmol) in anhydrous DMF (6.0 mL), was stirred at 60° C. for 5 h. Further tert-butyl 4-((methylsulfonyl)oxy)piperidine-1-carboxylate (390 mg) was added and the mixture stirred at 60° C. for 18 h. The reaction mixture was allowed to cool to room temperature and diluted with EtOAc (20 mL). The solution was washed sequentially with water (20 mL), 5% LiCl (aq) (20 mL) and brine (20 mL). The organic layer was passed through a hydrophobic frit and the filtrate evaporated in vacuo. The resulting oil was loaded in DCM (3 mL) and purified on a 24 g silica cartridge using a gradient of 0-40% EtOAc in cyclohexane over 12 column volumes. The appropriate fractions were combined and the solvent evaporated in vacuo to give the title compound as a light yellow solid (172 mg, 0.373 mmol). LCMS (formic A): Rt=1.59 min, M-^{t}Bu⁺=406, 408.

### Description 15

### tert-Butyl 4-(6-chloro-4-(2,4-dioxo-3-((2-(trimethylsilyl)ethoxy)methyl)tetrahydropyrimidin-1(2H)-yl)-1H-indazol-1-yl)piperidine-1-carboxylate (D15)

A mixture of copper(I) iodide (10 mg, 0.053 mmol), potassium carbonate (96 mg, 0.697 mmol) and 3-((2-(trimethylsilyl)ethoxy)methyl)dihydropyrimidine-2,4(1H,3H)-dione (may be prepared as described in Description 1; 102 mg, 0.418 mmol) was suspended in a solution of tert-butyl 4-(6-chloro-4-iodo-1H-indazol-1-yl)piperidine-1-carboxylate (may be prepared as described in Description 14; 161 mg, 0.349 mmol) in anhydrous 1,4-dioxane (3.5 mL). Trans-N,N′-Dimethylcyclohexane-1,2-diamine (0.016 mL, 0.105 mmol) was added, the vessel sealed and evacuated and purged with nitrogen (×3). The mixture was stirred at 120° C. in the sealed vessel for 16 h. The reaction was allowed to cool to room temperature, the suspension filtered through Celite and the Celite washed with EtOAc (10 mL). The filtrate was evaporated in vacuo and the residue loaded in DCM (3 mL) and purified on a 24 g silica cartridge using a gradient of 0-80% EtOAc in cyclohexane over 12 column volumes. The appropriate fractions were combined and the solvent evaporated in vacuo to give the title compound as a white solid (136 mg, 0.235 mmol, 68% yield). LCMS (high pH A): Rt=1.47 min, M−H⁻=576, 578.

### Description 16

### 4-Bromo-N,N-dimethyl-1H-indole-6-carboxamide (D16)

A mixture of 4-bromo-1H-indole-6-carboxylic acid (900 mg, 3.75 mmol) and HATU (2138 mg, 5.62 mmol) in anhydrous DMF (15 mL) was treated with DIPEA (1.310 mL, 7.50 mmol) and the solution left to stand at room temperature in a stoppered vessel for 20 min, then treated with 2 M dimethylamine solution in THF (12 mL, 24.00 mmol) and allowed to stand for 16 h. The reaction mixture was evaporated under vacuum (not to dryness) and the remaining solution diluted with EtOAc (30 mL). The solution was washed sequentially with water (30 mL), 1 M HCl (aq) (30 mL), saturated NaHCO₃ (aq) (30 mL) and brine (30 mL), and passed through a hydrophobic frit. A solid was collected on the frit and was suspended in water (10 mL), filtered, and washed with water (30 mL). This solid was added to the EtOAc filtrate from the hydrophobic frit and evaporated in vacuo. The solid was dissolved in MeOH (30 mL) and preadsorbed onto Florisil. The material was purified on a 40 g silica cartridge using a gradient of 0-90% EtOAc:ethanol (3:1) in TBME over 14 column volumes. The appropriate fractions were combined and the solvent evaporated in vacuo. The solid was dried under high vacuum for 16 h to give the title compound as an off-white solid (586 mg, 2.194 mmol, 59% yield). LCMS (high pH A): Rt=0.90 min, MH⁺=267, 269.

### Description 17

### Benzyl 4-(4-bromo-6-(dimethylcarbamoyl)-1H-indol-1-yl)piperidine-1-carboxylate (D17)

A mixture of 4-bromo-N,N-dimethyl-1H-indole-6-carboxamide (may be prepared as described in Description 16; 584 mg, 2.186 mmol), benzyl 4-((methylsulfonyl)oxy)piperidine-1-carboxylate (1400 mg, 4.47 mmol) and cesium carbonate (1400 mg, 4.30 mmol) in anhydrous DMF (12 mL), was evacuated and purged with nitrogen (×3). The reaction was stirred under nitrogen at 100° C. for 74 h. The reaction mixture was allowed to cool to room temperature and diluted with EtOAc (25 mL). The organic solution was washed sequentially with water (30 mL), 5% LiCl (aq) (30 mL) and brine (30 mL), and passed through a hydrophobic frit. The filtrate was evaporated in vacuo. The residue was loaded in DCM (10 mL) and purified on a 80 g silica cartridge using a gradient of 0-4% MeOH in DCM over 12 column volumes. The appropriate fractions were combined and the solvent evaporated in vacuo to give the title compound as a yellow gum (465 mg, 0.960 mmol, 44% yield). LCMS (high pH A): Rt=1.28 min, MH⁺=484, 486.

### Description 18

### Benzyl 4-(6-(dimethylcarbamoyl)-4-(2,4-dioxo-3-((2-(trimethylsilyl)ethoxy)methyl)tetrahydropyrimidin-1(2H)-yl)-1H-indol-1-yl)piperidine-1-carboxylate (D18)

A mixture of copper(I) iodide (12 mg, 0.063 mmol), potassium carbonate (170 mg, 1.228 mmol) and 3-((2-(trimethylsilyl)ethoxy)methyl)dihydropyrimidine-2,4(1H,3H)-dione (may be prepared as described in Description 1; 150 mg, 0.614 mmol) was diluted with a solution of benzyl 4-(4-bromo-6-(dimethylcarbamoyl)-1H-indol-1-yl)piperidine-1-carboxylate (may be prepared as described in Description 17; 315 mg, 0.650 mmol) in anhydrous 1,4-dioxane (4 mL). Trans-N,N′-Dimethylcyclohexane-1,2-diamine (0.019 mL, 0.123 mmol) was added, the vessel sealed, and evacuated and purged with nitrogen (×3). The mixture was stirred at 140° C. for 21 h and allowed to cool to room temperature. The lid was removed, and copper(I) iodide (12 mg, 0.063 mmol) and trans-N,N′-Dimethylcyclohexane-1,2-diamine (0.019 mL, 0.123 mmol) were added. The vessel was sealed, and evacuated and purged with nitrogen (×3). The mixture was stirred at 140° C. for 16 h. The reaction was allowed to cool to room temperature, the suspension filtered through Celite and the Celite washed with 1:1 MeOH:MeCN (10 mL). The filtrate was evaporated in vacuo and the residue loaded in DCM (3 mL) and purified on a 40 g silica cartridge using a gradient of 0-5% MeOH in DCM over 14 column volumes. The appropriate fractions were combined and the solvent evaporated in vacuo to give the title compound as an off white gum (190 mg, 0.293 mmol, 48% yield). LCMS (high pH A): Rt=1.31 min, MH⁺=648.

### Description 19

### 4-(2,4-Dioxo-3-((2-(trimethylsilyl)ethoxy)methyl)tetrahydropyrimidin-1(2H)-yl)-N,N-dimethyl-1-(piperidin-4-yl)-1H-indole-6-carboxamide (D19)

A flask containing 10 wt. % palladium on carbon (20 mg, 0.019 mmol) was evacuated and purged with nitrogen (×3) and a solution of benzyl 4-(6-(dimethylcarbamoyl)-4-(2,4-dioxo-3-((2-(trimethylsilyl)ethoxy)methyl)tetrahydropyrimidin-1(2H)-yl)-1H-indol-1-yl)piperidine-1-carboxylate (may be prepared as described in Description 18; 148 mg, 0.228 mmol) in Ethanol (12 mL) added.

The mixture was evacuated and purged with nitrogen (×3) and stirred under atmospheric pressure hydrogen at room temperature for 3 days. The reaction mixture was filtered and the filtrate evaporated in vacuo. The resulting gum was dissolved in MeOH (4 mL) and the solution was hydrogenated using a flow hydrogenator (settings: 60° C., 60 bar pressure, 1 mL/min) and 10% Pd/C CatCart 30 as the catalyst. The eluent was evaporated in vacuo to give the title compound as a light yellow gum (91 mg, 0.177 mmol). LCMS (formic A): Rt=0.71 min; MH⁺=514.

### Description 20

### Tert-butyl 4-(4-bromo-1H-pyrrolo[2,3-c]pyridin-1-yl)piperidine-1-carboxylate (D20)

A mixture of 4-bromo-1H-pyrrolo[2,3-c]pyridine (10 g, 50.8 mmol), tert-butyl 4-((methylsulfonyl)oxy)piperidine-1-carboxylate (28.4 g, 102 mmol) and cesium carbonate (33.1 g, 102 mmol) in DMF (100 mL) was heated at 100° C. for 16 h then allowed to stand at room temperature for 2 days. It was then treated with tert-butyl 4-((methylsulfonyl)oxy)piperidine-1-carboxylate (28.4 g, 102 mmol) and cesium carbonate (33.1 g, 102 mmol) and stirred at 100° C. for 4 h. The reaction mixture was evaporated in vacuo, then partitioned between water and EtOAc, washed with water and brine, dried (MgSO₄), filtered and evaporated to give a brown oil. Flash chromatography on 330 g silica eluting with 50-100% EtOAc in cyclohexane) gave after evaporation of the fractions the title compound as a colourless oil which solidified to a hard white solid on standing (15 g, 35.5 mmol, 70% yield). LCMS (high pH A): Rt=1.24 min, MH⁺=380, 382.

### Description 21

### Benzyl 4-(4-(2,4-dioxo-3-((2-(trimethylsilyl)ethoxy)methyl)tetrahydropyrimidin-1(2H)-yl)-1H-pyrazolo[4,3-c]pyridin-1-yl)piperidine-1-carboxylate (D21)

4-Bromo-1H-pyrazolo[4,3-c]pyridine (2.16 g, 10.91 mmol) and DMF (30 mL) were mixed and stirred at room temperature and treated with 60% NaH (0.873 g, 21.82 mmol). The mixture was stirred for 15 min, treated with benzyl 4-((methylsulfonyl)oxy)piperidine-1-carboxylate (3.14 g, 10.9 mmol) and heated to 80° C. After approximately 1 h, benzyl 4-((methylsulfonyl)oxy)piperidine-1-carboxylate (3.14 g, 10.9 mmol) was added. After an additional 1 h, benzyl 4-((methylsulfonyl)oxy)piperidine-1-carboxylate (3.14 g, 10.9 mmol) was added and the reaction mixture was stirred for 3 h at 80° C. The reaction mixture was diluted with EtOAc and NH₄Cl, washed with brine (twice), dried (MgSO₄), filtered and evaporated in vacuo. Purification by flash column chromatography (120 g, silica, 0-70% EtOAc/cyclohexane) gave after evaporation of the fractions a mixture of the N1 and N2 alkylated regioisomers as a brown oil (4.3 g). This was mixed with 3-((2-(trimethylsilyl)ethoxy)methyl)dihydropyrimidine-2,4(1H,3H)-dione (may be prepared as described in Description 1; 2.78 g, 11.39 mmol) potassium carbonate (2.86 g, 20.71 mmol), trans-N,N′-Dimethylcyclohexane-1,2-diamine (0.490 mL, 3.11 mmol), copper(I) iodide (0.296 g, 1.553 mmol) and anhydrous 1,4-dioxane (45 mL), degassed with vacuum/nitrogen several times, then heated at 135° C. for 36 h. The reaction mixture was diluted with water and EtOAc, washed with brine (twice), dried (MgSO₄), filtered and evaporated in vacuo to give a brown oil. Purification by flash column chromatography (silica, 120 g, 50-100% EtOAc/cyclohexane) followed by further chromatography (C18-silica, 150 g 40-90% (10 mM pH 10 NH₄CO₂H/water-MeCN) gave the title compound as a fine white powder (600 mg, 0.985 mmol, 10% yield). LCMS (high pH A): Rt=1.35 min, MH⁺=579.4.

### Description 22

### tert-Butyl 4-(4-bromo-1H-pyrazolo[3,4-c]pyridin-1-yl)piperidine-1-carboxylate (D22)

4-Bromo-1H-pyrazolo[3,4-c]pyridine (7.17 g, 36.2 mmol) DMF (100 mL) mixed and stirred at room temperature, treated with 60% NaH (2.90 g, 72.4 mmol), stirred 15 min, then treated with one-third of the tert-butyl 4-((methylsulfonyl)oxy)piperidine-1-carboxylate (30.3 g, 109 mmol), heated to 80° C. and then at approximately 1 h intervals, the remaining ⅔ was added. After 1 h, the reaction mixture was diluted with EtOAc and NH₄Cl and the layers were separated. The organic layer was washed with brine, dried, filtered, evaporated in vacuo and purified using a 330 g silica column, eluting with 0-100% EtOAc:cyclohexane, and the product-containing fractions were combined and concentrated in vacuo. The resulting yellow oil was divided into five, diluting with DMSO-MeOH (1:1, ca 20 mL), and each batch was further purified using a 150 g C18 column, eluting with 30-85% MeCN: 10 mM aqueous ammonium bicarbonate solution, and the desired fractions were combined and concentrated in vacuo to give the title compound (6.1 g, 15.20 mmol, 42% yield). LCMS (high pH A): Rt=1.26 min, MH⁺=381, 383.

### Description 23

### tert-Butyl 4-(4-(2,4-dioxo-3-((2-(trimethylsilyl)ethoxy)methyl)tetrahydropyrimidin-1(2H)-yl)-1H-pyrazolo[3,4-c]pyridin-1-yl)piperidine-1-carboxylate (D23)

3-((2-(Trimethylsilyl)ethoxy)methyl)dihydropyrimidine-2,4(1H,3H)-dione (may be prepared as described in Description 1; 3.91 g, 16.00 mmol), potassium carbonate (4.42 g, 32.0 mmol), tert-butyl 4-(4-bromo-1H-pyrazolo[3,4-c]pyridin-1-yl)piperidine-1-carboxylate (may be prepared as described in Description 22; 6.1 g, 16.00 mmol), trans-N,N-dimethylcyclohexane-1,2-diamine (0.757 mL, 4.80 mmol), copper(I) iodide (0.457 g, 2.400 mmol) and anhydrous 1,4-dioxane (150 mL) were mixed, degassed with N₂ for 10 min, then heated at 120° C. (heating block temperature) for 36 h. The resulting mixture was filtered through celite, evaporated in vacuo, taken up in EtOAc, and washed with water and brine, dried (MgSO₄), filtered and evaporated in vacuo to give a green oil. The crude product was purified using an 80 g silica column, eluting with, 0-8% 4 M NH₃-MeOH):DCM, and the desired fractions were combined and concentrated in vacuo to give the title compound as a pale yellow sticky gum (4.5 g, 7.43 mmol, 47% yield). LCMS (high pH A): Rt=1.27 min, MH⁺=545.

### Description 24

### Benzyl 4-(6-chloro-4-nitro-1H-indol-1-yl)piperidine-1-carboxylate (D24)

A mixture of 6-chloro-4-nitro-1H-indole (900 mg, 4.58 mmol), benzyl 4-((methylsulfonyl)oxy)piperidine-1-carboxylate (2.15 g, 6.86 mmol) and cesium carbonate (2.25 g, 6.91 mmol) in anhydrous DMF (10 mL), was evacuated and purged with nitrogen (×3). The reaction was stirred under nitrogen at 85° C. for 40 h. Further benzyl 4-((methylsulfonyl)oxy)piperidine-1-carboxylate (2.15 g, 6.86 mmol) and cesium carbonate (2.25 g, 6.91 mmol) were added and reaction stirred under nitrogen at 100° C. for 3 days. The reaction mixture was diluted with EtOAc (50 mL) and washed sequentially with water (50 mL) and brine (25 mL). The organic phase was dried (MgSO₄), filtered and evaporated to dryness. The resulting residue was dissolved in DCM and purified by flash column chromatography (Silica, 80 g, eluting with 14 column volumes over a gradient of 0-80% Cyclohexane/EtOAc). The appropriate fractions were combined and evaporated to dryness to give the title compound as a brown solid (1.15 g, 2.78 mmol) which was used crude in the next step. LCMS (high pH A): Rt=1.44 min, MH⁺=414.

### Description 25

### Benzyl 4-(4-amino-6-chloro-1H-indol-1-yl)piperidine-1-carboxylate (D25)

A solution of benzyl 4-(6-chloro-4-nitro-1H-indol-1-yl)piperidine-1-carboxylate (may be prepared as described in Description 24; 1.15 g, 2.78 mmol), iron (0.776 g, 13.89 mmol) and ammonium chloride (1.189 g, 22.23 mmol) in ethanol (12 mL) and water (6.00 mL) was stirred at 90° C. for 2 h. The reaction mixture was allowed to cool to room temperature and filtered through a 10 g Celite cartridge, washing with ethanol. The filtrate was evaporated, diluted with EtOAc (100 mL) and washed sequentially with water (100 mL) and brine (100 mL). The organic layer was then evaporated to dryness to give the title compound as a dark brown gum (268 mg, 0.698 mmol) which was used crude in the next step. LCMS (high pH A): Rt=1.26 min, MH⁺=384.

### Description 26

### Benzyl 4-(6-chloro-4-(2,4-dioxotetrahydropyrimidin-1(2H)-yl)-1H-indol-1-yl)piperidine-1-carboxylate (D26)

Benzyl 4-(4-amino-6-chloro-1H-indol-1-yl)piperidine-1-carboxylate (may be prepared as described in Description 25; 268 mg, 0.698 mmol) was dissolved in Toluene (1 mL) and treated with acrylic acid (0.144 mL, 2.094 mmol). The mixture was stirred for 2.5 h at 50° C. then at 70° C. for 4 h, then at 40° C. for 24 h. More acrylic acid (0.048 mL) was added and the mixture was stirred at 70° C. for 5 h. The reaction mixture was evaporated evaporated, taken up in acetic acid (2.5 mL), treated with urea (189 mg, 3.14 mmol) and heated for 24 h at 140° C. The reaction mixture was evaporated in vacuo and partitioned between saturated sodium bicarbonate (10 mL) and EtOAc (50 mL). The organic phase was washed sequentially with water (50 mL) and brine (40 mL) and evaporated to dryness to give a dark brown gum. This was dissolved in DCM (3 mL) and purified by flash column chromatography (silica, 40 g, eluting with 14 column volumes using a gradient of 0-80% 3:1 EtOAc:Ethanol in TBME). The appropriate fractions were combined and evaporated to dryness to yield the title compound as an orange solid (98 mg, 0.137 mmol, 20% yield). LCMS (high pH A): Rt=1.14 min, MH⁺=481.

### Description 27

### Benzyl 4-(4-amino-6-fluoro-1H-indazol-1-yl)piperidine-1-carboxylate (D27)

6-Fluoro-1H-indazol-4-amine (3 g, 19.85 mmol) and DMF (30 mL) were mixed and stirred at room temperature, treated with NaH (1.588 g, 39.7 mmol), stirred 15 min, then benzyl 4-((methylsulfonyl)oxy)piperidine-1-carboxylate (6.22 g, 19.8 mmol) and heated to 80° C. After approximately 1 h benzyl 4-((methylsulfonyl)oxy)piperidine-1-carboxylate (6.22 g, 19.8 mmol) was added. After an additional 1 h, benzyl 4-((methylsulfonyl)oxy)piperidine-1-carboxylate (6.22 g, 19.8 mmol) was added and the reaction mixture heated to 80° C. for 3 h. The reaction mixture was diluted with EtOAc and NH₄Cl, washed with brine (twice), dried (MgSO₄), filtered, evaporated in vacuo and purified by flash column chromatography (120 g, silica, 0-70% EtOAc/cyclohexane) to give after evaporation of the fractions the title compound as a colourless gum (2.1 g, 5.70 mmol, 29% yield). LCMS (high pH A): Rt=1.11 min, MH⁺=369.2.

### Description 28

### Tert-butyl 4-fluoro-4-formylpiperidine-1-carboxylate (D28)

Dess-Martin periodinane (635 mg, 1.498 mmol) was added portionwise to a stirred solution of tert-butyl 4-fluoro-4-(hydroxymethyl)piperidine-1-carboxylate (obtainable from Porse Fine Chemical Co. Ltd.; 233 mg, 0.999 mmol) in DCM (5 mL). After complete addition the reaction mixture was stirred at room temperature for 1.5 h. Saturated sodium bicarbonate solution (10 mL) was added and the mixture was stirred for 10 min. The organic phase was separated. The aqueous phase was extracted with DCM (2×5 mL). The combined organics were dried and evaporated to give the title compound as a colourless solid (231 mg, 0.999 mmol, 100% yield).

### Description 29

### Tert-butyl 4-((4-(4-(2,4-dioxotetrahydropyrimidin-1(2H)-yl)-1H-pyrrolo[2,3-c]pyridin-1-yl)piperidin-1-yl)methyl)-4-fluoropiperidine-1-carboxylate (D29)

A mixture of 1-(1-(piperidin-4-yl)-1H-pyrrolo[2,3-c]pyridin-4-yl)dihydropyrimidine-2,4(1H,3H)-dione (may be prepared as described in Example CB6; 100 mg, 0.319 mmol) and tert-butyl 4-fluoro-4-formylpiperidine-1-carboxylate (may be prepared as described in Description 28; 148 mg, 0.638 mmol) in DCM (5 mL) was stirred at room temperature for 1 h. Triethylamine (65 mg, 0.09 mL, 0.642 mmol) was added followed by sodium triacetoxyborohydride (203 mg, 0.957 mmol). The reaction mixture was stirred at room temperature for 1 h then allowed to stand for 16 h. Saturated sodium bicarbonate solution (10 mL) was added. The mixture was stirred for 10 min. The organic phase was separated. The aqueous phase was extracted with DCM (5 mL). The combined organics were dried and evaporated. The residue was chromatographed [0-20% ethanol/EtOAc] to give the title compound as a colourless solid (90 mg, 0.170 mmol, 53% yield). LCMS (high pH A): Rt=1.04 min, MH⁺=529

### Description 30

### Tert-butyl 2-(4-(4-(2,4-dioxotetrahydropyrimidin-1(2H)-yl)-1H-indol-1-yl)piperidin-1-yl)acetate (D30)

A mixture of 1-(1-(piperidin-4-yl)-1H-indol-4-yl)dihydropyrimidine-2,4(1H,3H)-dione (may be prepared as described in Example CB1; 500 mg, 1.601 mmol), tert-butyl 2-bromoacetate (0.25 mL, 1.693 mmol) and triethylamine (0.45 mL, 3.23 mmol) were dissolved in anhydrous DMF (9 mL). The reaction vessel was then sealed and evacuated and purged with nitrogen (×3) before being left to stir at room temperature for 4 h. The mixture was diluted with EtOAc (30 mL) and washed with brine (2×20 mL). Solid formed between partitions and was isolated and dried to give the title compound as a white powder (433 mg, 1.015 mmol, 63% yield). LCMS (high pH A): Rt=1.04 min, MH⁺=427

### Description 31

### tert-Butyl 4-(4-(4-(2,4-dioxotetrahydropyrimidin-1(2H)-yl)-1H-indol-1-yl)piperidin-1-yl)benzoate (D31)

A mixture of 1-(1-(piperidin-4-yl)-1H-indol-4-yl)dihydropyrimidine-2,4(1H,3H)-dione (may be prepared as described in Example ECB1; 150 mg, 0.480 mmol) and tert-butyl 4-fluorobenzoate (119 mg, 0.606 mmol) in anhydrous N-Methyl-2-pyrrolidone (NMP) (3500 μL) in a 2.0-5.0 mL vial was treated with DIPEA (210 μL, 1.200 mmol). The vial was sealed, stirred and heated conventionally at 140° C. for 88 h. The reaction mixture was then diluted in water (10 mL) before being extracted with EtOAc (50 mL) and washed with brine (30 mL). The organic layer was then passed through a hydrophobic frit before the filtrate was evaporated using rotary evaporation. The resulting sample was loaded in DCM (5 mL) and purified on a 40 g silica cartridge using a gradient of 0-100% EtOAc in cyclohexane over 14 column volumes. The appropriate fractions were combined and the solvent removed by rotary evaporation to give the title compound as a white solid (60.8 mg, 0.124 mmol, 26% yield). LCMS (high pH A): Rt=1.27 min, MH⁺=489

### Description 32

### Methyl 4-bromo-1-isopropyl-1H-indole-6-carboxylate (D32)

A mixture of methyl 4-bromo-1H-indole-6-carboxylate (30 g, 118 mmol) and cesium carbonate (115 g, 354 mmol) in CH₃CN (500 mL) was treated with 2-iodopropane (23.61 mL, 236 mmol) and stirred at 80° C. for 16 h. The reaction mixture was filtered and concentrated. The brown residue was partitioned between sat. NaHCO₃ (aq) and EtOAc. The organic layer was washed with brine, dried over MgSO₄ and concentrated to a brown residue. This was purified on a 300 g silica column using a gradient of 5-50% EtOAc in hexane to give the title compound as a yellow solid (29.22 g, 99 mmol, 84% yield). LCMS (TFA A): Rt=1.27 min, MH⁺=296, 298

### Description 33

### Methyl 4-amino-1-isopropyl-1H-indole-6-carboxylate (D33)

Methyl 4-bromo-1-isopropyl-1H-indole-6-carboxylate (may be prepared as described in Description 32; 15.3 g, 51.7 mmol), cesium carbonate (42.1 g, 129 mmol), BINAP (6.43 g, 10.33 mmol) and Pd₂(dba)₃ (4.73 g, 5.17 mmol) were mixed in toluene (250 mL), and benzophenone imine (10.40 mL, 62.0 mmol) added. The mixture was stirred at 110° C. for 16 h. The mixture was cool to room temperature and filtered. The filtrate was partitioned between sat. NaHCO₃ (aq) and EtOAc. The organic layer was washed by brine, dried over MgSO₄ and concentrated to a brown residue. The residue was dissolved in THF (300 mL), and 3 N HCl (aq) (34.4 mL, 103 mmol) was added. The mixture was stirred at room temperature for 2 h. Na₂CO₃ was added to the mixture portionwise until pH˜9. The mixture was then partitioned between saturated NaHCO₃ (aq) and EtOAc. The organic layer was washed by brine, dried over MgSO₄ and concentrated to a brown residue. The residue was purified on a 330 g silica column using a gradient of 5-50% 3:1 EtOAc:ethanol in hexane to give the title compound as a yellow oil, which solidified into a yellow foam under high vacuum (7.48 g, 32.2 mmol, 62% yield). LCMS (formic A): Rt=0.68 min, MH⁺=233

### Description 34

### 4-Amino-1-isopropyl-1H-indole-6-carboxylic acid (D34)

Methyl 4-amino-1-isopropyl-1H-indole-6-carboxylate (may be prepared as described in Description 33; 7.48 g, 32.2 mmol) was dissolved in THF (50 mL), and then NaOH (2.5 M in H₂O) (38.6 mL, 97 mmol) was added and heated at 70° C. for 16 h. The mixture was cooled to room temperature and partitioned between water (100 mL) and EtOAc (100 mL). The organic layer contained impurity and was discarded. To the aqueous layer was added conc. HCl (aq) (40 mL) dropwise to pH˜5. The resulting black oil was extracted by EtOAc. The organic layer was washed with brine, dried over MgSO₄, and concentrated to give the title compounds as a brown residue (6.23 g, 28.5 mmol, 89% yield). LCMS (TFA A): Rt=0.43 min, MH⁺=219

### Description 35

### 4-(2,4-dioxotetrahydropyrimidin-1(2H)-yl)-1-isopropyl-1H-indole-6-carboxylic acid (D35)

4-Amino-1-isopropyl-1H-indole-6-carboxylic acid (may be prepared as described in Description 34; 7.23 g, 33.1 mmol) was suspended in toluene (100 mL). Acrylic acid (6.82 mL, 99 mmol) was added and heated at 40° C. for 16 h. Further acrylic acid (6.82 mL, 99 mmol) was added and heated at 60° C. for 48 h. The mixture was concentrated to give a mixture of 4-((2- carboxyethyl)amino)-1-isopropyl-1H-indole-6-carboxylic acid and 3,3′-((6-carboxy-1-isopropyl-1H-indol-4-yl) azanediyl)dipropionic acid (9.62 g) as a black oil. The mixture was dissolved in acetic acid (50 mL) and urea (9.95 g, 166 mmol) added, and heated at 130° C. for 16 h. The mixture was cooled to room temperature and added to rapidly stirred water (250 mL). The resulting off-white precipitate was filtered and dried under vacuum to give the title compound as a light brown solid (8.07 g, 25.6 mmol, 77% yield). LCMS (formic A): Rt=0.63 min, MH⁺=316

### Description 36

### tert-Butyl 4-(4-(2,4-dioxotetrahydropyrimidin-1(2H)-yl)-1-isopropyl-1H-indole-6-carbonyl)piperazine-1-carboxylate (D36)

A mixture of 4-(2,4-dioxotetrahydropyrimidin-1(2H)-yl)-1-isopropyl-1H-indole-6-carboxylic acid (may be prepared as described in Description 35; 3.5 g, 11.10 mmol), tert-butyl piperazine-1-carboxylate (2.89 g, 15.54 mmol) and triethylamine (7.74 mL, 55.5 mmol) in DMF (20 mL) was treated with HATU (5.91 g, 15.54 mmol) and stirred at ambient temperature for 2 h. The mixture was partitioned between EtOAc (120 mL) and brine (50 mL). The organic phase was washed with further brine (3×50 mL), dried (MgSO₄), filtered and evaporated to dryness. The product was purified by chromatography on silica using a gradient elution from 0% to 10% MeOH in DCM to give the title compound (4.3 g, 8.89 mmol, 80% yield). LCMS (formic A): Rt=0.96 min, M-^{t}Bu⁺=428

### Description 37

### tert-Butyl 4-(4-(2,4-dioxotetrahydropyrimidin-1(2H)-yl)-1-isopropyl-N-methyl-1H-indole-6-carboxamido)piperidine-1-carboxylate (D37)

A mixture of 4-(2,4-dioxotetrahydropyrimidin-1(2H)-yl)-1-isopropyl-1H-indole-6-carboxylic acid (may be prepared as described in Description 35; 2 g, 6.34 mmol), tert-butyl 4-(methylamino)piperidine-1-carboxylate (1.903 g, 8.88 mmol) and triethylamine (4.42 mL, 31.7 mmol) in DMF (10 mL) was treated with HATU (3.38 g, 8.88 mmol) and stirred at ambient temperature for 2 h. The mixture was partitioned between EtOAc (120 mL) and brine (50 mL). The organic phase was washed with further brine (3×50 mL), dried, filtered and evaporated to dryness. The product was purified by chromatography on C18 silica using a gradient elution from 30% to 70% MeCN in 10 mM aqueous ammonium bicarbonate (pH10) to give the title compound (2.6 g, 5.08 mmol, 80% yield). LCMS (high pH A): Rt=0.98 min, M+Na⁺=534 M-Boc+H⁺=412.

### Description 38 tert-Butyl 4-((4-(2,4-dioxotetrahydropyrimidin-1(2H)-yl)-1-isopropyl-N-methyl-1H-indole-6-carboxamido)methyl)piperidine-1-carboxylate (D38)

A mixture of 4-(2,4-dioxotetrahydropyrimidin-1(2H)-yl)-1-isopropyl-1H-indole-6-carboxylic acid (may be prepared as described in Description 35; 2 g, 6.34 mmol), tert-butyl 4-((methylamino)methyl)piperidine-1-carboxylate (2.028 g, 8.88 mmol) and triethylamine (4.42 mL, 31.7 mmol) in DMF (10 mL) was treated with HATU (3.38 g, 8.88 mmol) and stirred at ambient temperature for 2 h. The mixture was partitioned between EtOAc (120 mL) and brine (50 mL). The organic phase was washed with further brine (3×50 mL), dried, filtered and evaporated to dryness. The product was purified by chromatography on silica using a gradient elution from 0% to 10% MeOH in DCM to afford the title compound (2.72 g, 5.17 mmol, 82% yield). LCMS (formic A): Rt=1.02 min, M-Boc+H⁺=426.

### Description 39

### tert-Butyl 9-(4-(2,4-dioxotetrahydropyrimidin-1(2H)-yl)-1-isopropyl-1H-indole-6-carbonyl)-2,9-diazaspiro[5.5]undecane-2-carboxylate (D39)

HATU (149 mg, 0.392 mmol) was added carefully to a mixture of DIPEA (0.170 mL, 0.980 mmol), 4-(2,4-dioxotetrahydropyrimidin-1(2H)-yl)-1-isopropyl-1H-indole-6-carboxylic acid (may be prepared as described in Description 35; 103 mg, 0.327 mmol) and tert-butyl 2,9-diazaspiro[5.5]undecane-2-carboxylate (100 mg, 0.392 mmol) in DMF (5 mL). The reaction mixture was stirred at room temperature for 2 h. The reaction mixture was diluted in EtOAc (20 mL) and quenched with sodium bicarbonate solution (15 mL). The organic layer was separated and washed with 1 M hydrochloric acid (15 mL) then brine (15 mL). The organic layer was dried and evaporated to give the title compound as a brown gum (204 mg, 0.370 mmol). LCMS (high pH A): Rt=1.11 min, M-^{t}Bu⁺=496

### Description 40

### tert-Butyl 9-(4-(2,4-dioxotetrahydropyrimidin-1(2H)-yl)-1-isopropyl-1H-indole-6-carbonyl)-3,9-diazaspiro[5.5]undecane-3-carboxylate hydrochloride (D40)

HATU (145 mg, 0.381 mmol) was added carefully to a mixture of DIPEA (0.165 mL, 0.951 mmol), 4-(2,4-dioxotetrahydropyrimidin-1(2H)-yl)-1-isopropyl-1H-indole-6-carboxylic acid (may be prepared as described in Description 35; 100 mg, 0.317 mmol) and tert-butyl 3,9-diazaspiro[5.5]undecane-3-carboxylate (97 mg, 0.381 mmol) in DMF (5 mL). The reaction mixture was stirred at room temperature for 90 min. The reaction mixture was diluted in EtOAc (20 mL) and quenched with sodium bicarbonate solution (15 mL). The organic layer was separated and washed with 1 M hydrochloric acid (15 mL) then brine (15 mL). The organic layer was dried and evaporated to give the title compound as a brown gum (191 mg, 0.346 mmol). LCMS (high pH A): Rt=0.68 min, MH⁺=452

### Description 41

### tert-Butyl 2-(4-(2,4-dioxotetrahydropyrimidin-1(2H)-yl)-1-isopropyl-1H-indole-6-carbonyl)-2,8-diazaspiro[4.5]decane-8-carboxylate (D41)

HATU (145 mg, 0.381 mmol) was added carefully to a mixture of DIPEA (0.165 mL, 0.951 mmol), 4-(2,4-dioxotetrahydropyrimidin-1(2H)-yl)-1-isopropyl-1H-indole-6-carboxylic acid (may be prepared as described in Description 35; 100 mg, 0.317 mmol), and tert-butyl 2,8-diazaspiro[4.5]decane-8-carboxylate (91 mg, 0.381 mmol) in DMF (5 mL). The reaction mixture was stirred at RT for 90 min. The reaction mixture was diluted in EtOAc (20 mL) and quenched with sodium bicarbonate solution (15 mL). The organic layer was separated and washed with 1 M hydrochloric acid (15 mL) then brine (15 mL). The organic layer was dried and evaporated. The residue was chromatographed [0-100% EtOAc/cyclohexane, 0-10% ethanol/EtOAc] to give the title compound as a brown solid (60 mg, 0.112 mmol, 35% yield). LCMS (high pH A): Rt=1.05 min, M-^{t}Bu⁺=482

### Description 42

### tert-Butyl ((1r,4r)-4-(3-chloro-4-cyanophenoxy)cyclohexyl)carbamate (D42)

To a stirred solution of tert-butyl ((1r,4r)-4-hydroxycyclohexyl)carbamate (15.3 g, 71.1 mmol) in DMF (100 mL), cooled in an ice-water bath, was added 60% NaH in mineral oil (3.70 g, 92 mmol) and the reaction mixture was stirred for 20 min, then 2-chloro-4-fluorobenzonitrile (12.16 g, 78 mmol) was added in small portions (effervescence) and the mixture stirred for 2 h, allowing it to warm to room temperature. The mixture was quenched with water (100 mL) and the resulting suspension stirred for 30 min, then the product collected by filtration and washed with water to give a pale yellow solid. The crude was dissolved in DCM (100 mL) and washed with water (100 mL), then the organics dried and evaporated in vacuo to give the title compound as a colourless solid (28.6 g, 82 mmol). Material carried through to the next step without purification. LCMS (formic A): Rt=1.33 min, M-^{t}Bu⁺=295, 297.

### Description 43

### tert-Butyl ((1r,4r)-4-(4-cyano-3-(trifluoromethyl)phenoxy)cyclohexyl)carbamate (D43)

To a stirred solution of 4-fluoro-2-(trifluoromethyl)benzonitrile (5.27 g, 27.9 mmol) and tert-butyl ((1r,4r)-4-hydroxycyclohexyl)carbamate (5 g, 34.8 mmol) in DMF (100 mL), in an ice bath, was added 60% NaH in mineral oil (1.393 g, 34.8 mmol), and the reaction mixture was stirred for 3 h under a nitrogen atmosphere. The reaction mixture was diluted with water (100 mL) and extracted with EtOAc (3×200 mL). The organic layers were combined, passed through a hydrophobic frit, and the solvent removed in vacuo. The residue was split into two batches, which were both purified by normal phase column chromatography (0-30%, EtOAc in cyclohexane, 330 g silica, 10 column volumes), then combined to give the title compound as a white solid (7.32 g, 19.04 mmol, 82% yield). LCMS (high pH A): Rt=1.34 min, M-Boc+285.

### Description 44

### tert-Butyl ((1r,3r)-3-(3-chloro-4-cyanophenoxy)-2,2,4,4-tetramethylcyclobutyl)carbamate (D44)

To a stirred solution of tert-butyl ((1r,3r)-3-hydroxy-2,2,4,4-tetramethylcyclobutyl)carbamate (1 g, 4.11 mmol) in DMF (100 mL), cooled in an ice-water bath, was added 60% NaH in mineral oil (0.214 g, 5.34 mmol) and the reaction mixture was stirred for 20 min, then 2-chloro-4-fluorobenzonitrile (0.703 g, 4.52 mmol) was added in small portions (effervescence) and the mixture stirred for 2 h, allowing it to warm to room temperature. The mixture was quenched with water (100 mL) and the resulting suspension stirred for 30 min, then the product collected by filtration and washed with water to give a pale yellow solid. The crude was dissolved in DCM and loaded onto a 40 g silica column, then eluted with 0-50% EtOAc/cyclohexane and product-containing fractions evaporated in vacuo to give the title compound as a colourless gum (1.58 g, 4.17 mmol, 101% yield). LCMS (formic A): Rt=1.49 min, M-^{t}Bu⁺=323, 325.

### Description 45

### 4-(((1r,4r)-4-Aminocyclohexyl)oxy)-2-chlorobenzonitrile hydrochloride (D45)

To tert-butyl ((1r,4r)-4-(3-chloro-4-cyanophenoxy)cyclohexyl)carbamate (may be prepared as described in Description 42; 7.1204 g, 20.30 mmol) was added 4 M HCl in dioxane (25.4 mL, 101 mmol) and the reaction mixture was stirred at room temperature for 35 min. The solvent was removed in vacuo to afford the title compound as a white solid (6.59 g, 20.42 mmol, 101% yield).

LCMS (high pH A): Rt=0.93 min, MH⁺=251, 253.

### Description 46

### 4-(((1r,4r)-4-Aminocyclohexyl)oxy)-2-chlorobenzonitrile (D46)

tert-Butyl ((1r,4r)-4-(3-chloro-4-cyanophenoxy)cyclohexyl)carbamate (may be prepared as described in Description 42; 24 g, 68.4 mmol) was dissolved in DCM (100 mL) and TFA (26.4 mL, 342 mmol) was added, then the mixture was stirred for 2 h at room temperature. The solvent was evaporated in vacuo and the residue partitioned between 1 M HCl and MTBE (200 mL each). The aqueous layer was basified with potassium carbonate and extracted with DCM (2×200 mL), the organics dried and evaporated in vacuo to give the title compound as a colourless liquid (16.8 g, 67.0 mmol, 98% yield). LCMS (high pH A): Rt=0.91 min, MH⁺=251, 253

### Description 47

### 4-(((1r,4r)-4-Aminocyclohexyl)oxy)-2-chlorobenzonitrile 2,2,2-trifluoroacetate (D47)

A solution of tert-butyl ((1r,4r)-4-(3-chloro-4-cyanophenoxy)cyclohexyl)carbamate (may be prepared as described in Description 42; 20.0 g, 57.0 mmol) in DCM (80 mL) was cooled to 0° C. A solution of TFA (20 mL) in DCM (20 mL) was added slowly. After complete addition the reaction mixture was allowed to warm to room temperature and stirred for 18 h. TFA (20 mL) was added slowly and the reaction mixture was stirred at RT for 2 h. The reaction mixture was diluted with diethyl ether (200 mL) and stirred for 30 min. The precipitate was filtered off, washed with diethyl ether and dried to give the title compound as an off white solid (18.02 g, 49.4 mmol, 87% yield). LCMS (high pH A): Rt=0.91 min, MH⁺=251, 253.

### Description 48

### 4-(((1r,4r)-4-Aminocyclohexyl)oxy)-2-(trifluoromethyl)benzonitrile hydrochloride (D48)

To tert-butyl ((1r,4r)-4-(4-cyano-3-(trifluoromethyl)phenoxy)cyclohexyl)carbamate (may be prepared as described in Description 43; 7.32 g, 19.04 mmol) was added 4 M HCl in dioxane (23.80 mL, 95 mmol), and the reaction mixture was stirred at room temperature for 10 min. The solvent was removed in vacuo to give the title compound (6.67 g, 19.34 mmol, 102% yield) as a white solid. Carried through to the next step without purification. LCMS (high pH A): Rt=0.99 min, MH⁺=285.

### Description 49

### 4-((1r,3r)-3-Amino-2,2,4,4-tetramethylcyclobutoxy)-2-chlorobenzonitrile (D49)

TFA (3 mL, 38.9 mmol) was added to a solution of tert-butyl ((1r,3r)-3-(3-chloro-4-cyanophenoxy)-2,2,4,4-tetramethylcyclobutyl)carbamate (may be prepared as described in Description 44; 1.58 g, 4.17 mmol) in DCM (10 mL) and the mixture was stirred at room temperature for 2 h, then evaporated in vacuo. The gummy residue was stirred in water and 1 M HCl (20 mL each), giving a dense suspension. The mixture was basified with solid potassium carbonate and extracted with DCM (3×20 mL), the organics dried and evaporated in vacuo to give the title compound as a colourless solid (1.05 g, 3.77 mmol, 90% yield). Carried through to the next step without purification. LCMS (formic A): Rt=0.64 min, MH⁺=279, 281.

### Description 50

### 4-((1r,3r)-3-Amino-2,2,4,4-tetramethylcyclobutoxy)-2-(trifluoromethyl)benzonitrile hydrochloride (D50)

A solution of tert-butyl ((1r,3r)-3-(4-cyano-3-(trifluoromethyl)phenoxy)-2,2,4,4-tetramethylcyclobutyl)carbamate (may be prepared as described in Description 186; 1 g, 2.425 mmol) in 1,4-dioxane (6 mL) was treated with hydrogen chloride (4 M in 1, 4-dioxane) (6 mL, 24.00 mmol) and stirred at ambient temperature for 6 h. The mixture was blown to dryness with a stream of nitrogen to give the title compound. LCMS (formic A): Rt=0.69 min, MH⁺=313.

### Description 51

### Methyl 5-(4-(hydroxymethyl)piperidin-1-yl)pyrazine-2-carboxylate (D51)

A solution of methyl 5-chloropyrazine-2-carboxylate (5 g, 29.0 mmol) and piperidin-4-ylmethanol (4.00 g, 34.8 mmol) in DCM (40 mL) was treated with triethylamine (8.08 mL, 57.9 mmol), and stirred at 60° C. for 4 h. The mixture was partitioned between DCM (50 mL) and water (20 mL), and the organic phase was evaporated to dryness to give the title compound (6.6 g, 26.3 mmol, 91% yield). LCMS (formic A): Rt=0.62 min, MH⁺=252

### Description 52

### Methyl 6-(4-(hydroxymethyl)piperidin-1-yl)pyridazine-3-carboxylate (D52)

A solution of methyl 6-chloropyridazine-3-carboxylate (6 g, 34.8 mmol) and piperidin-4-ylmethanol (5 g, 43.4 mmol) in MeCN (100 mL) was treated with triethylamine (5 mL, 35.9 mmol), and stirred at ambient temperature for 18 h. The mixture was evaporated in vacuo and the residue dissolved in DCM and washed with water. The solvent was dried and evaporated in vacuo to give the title compound as a grey solid (5.4 g, 21.49 mmol, 62% yield). LCMS (formic A): Rt=0.50 min, MH⁺=252.

### Description 53

### Ethyl 2-(4-(hydroxymethyl)piperidin-1-yl)pyrimidine-5-carboxylate (D53)

A solution of ethyl 2-chloropyrimidine-5-carboxylate (5 g, 26.8 mmol) and piperidin-4-ylmethanol (3.70 g, 32.2 mmol) in DCM (40 mL) was treated with triethylamine (7.47 mL, 53.6 mmol), and stirred at 60° C. for 4 h. The mixture was partitioned between DCM (50 mL) and water (20 mL), the organic phase was evaporated to dryness to give the title compound (6.8 g, 25.6 mmol, 96% yield).

LCMS (formic A): Rt=0.86 min, MH⁺=266.

### Description 54

### Methyl 6-(4-(hydroxymethyl)piperidin-1-yl) nicotinate (D54)

A solution of methyl 6-chloronicotinate (5.5 g, 32.1 mmol) and piperidin-4-ylmethanol (4.43 g, 38.5 mmol) in Chloroform (60 mL) was treated with DIPEA (11.20 mL, 64.1 mmol), and stirred at 60° C. for 4 h. The mixture was partitioned between DCM (50 mL) and water (20 mL), and the organic phase was evaporated to dryness to give a pale yellow solid The crude was dissolved in DCM and loaded onto a 120 g silica column, then eluted with 0-100% EtOAc/cycloehxane and product-containing fractions evaporated in vacuo to give the title compound as a colourless solid (5.2 g, 20.78 mmol, 65% yield). LCMS (high pH A): Rt=0.80 min, MH⁺=251.

### Description 55

### Methyl 6-(1,4-dioxa-8-azaspiro[4.5]decan-8-yl)pyridazine-3-carboxylate (D55)

A mixture of methyl 6-chloropyridazine-3-carboxylate (1.7 g, 9.85 mmol), 1,4-dioxa-8-azaspiro[4.5]decane (1.25 mL, 9.75 mmol) and DIPEA (3.5 mL, 20.04 mmol) was dissolved in DMSO (13 mL). The reaction mixture was then sealed within a microwave vessel and heated in a Biotage Initiator microwave for 45 min at 90° C. using a high absorption setting. The reaction mixture was allowed to cool to room temperature before being diluted with water (50 mL), extracted with EtOAc (100 mL) and then washed with Brine (30 mL). The organic layer was then passed through a hydrophobic frit and the filtrate evaporated to dryness to give the title compound as an off white powder (2.2 g, 7.88 mmol). LCMS (formic A): Rt=1.66 min, MH⁺=280.

### Description 56

### Methyl 6-(4-(2-((tert-butyldimethylsilyl)oxy)ethyl)piperidin-1-yl)pyridazine-3-carboxylate (D56)

DIPEA (3.34 mL, 19.12 mmol) was added to a mixture of crude 4-(2-((tert-butyldimethylsilyl)oxy)ethyl)piperidine (2.54 g, 10.43 mmol) and methyl 6-chloropyridazine-3-carboxylate (1.5 g, 8.69 mmol) in DMSO (11 mL). The mixture was stirred at 90° C. in a Biotage microwave for 45 min (cooling-off; FHT-on; absorbance-high). A precipitate had formed which was diluted with water (20 mL) and filtered. The solid was washed with water (200 mL), filtered and dried in a vacuum oven for 16 h to give the title compound as tan crystals (2.74 g, 7.22 mmol, 83% yield). LCMS (formic A): Rt=1.45 min, MH⁺=380.

### Description 57

### Methyl 4-(3-hydroxypropyl)benzoate (D57)

Palladium (II) acetate (112 mg, 5 mol %) was added to a stirred mixture of methyl 4-iodobenzoate (2.62 g, 10.0 mmol), allyl alcohol (1.162 g, 1.36 mL, 20 mmol), sodium bicarbonate (2.52 g, 30.0 mmol) and benzyl triethylammonum chloride (2.278 g, 10 mmol) in dry MeCN (40 mL) under nitrogen. The reaction mixture was stirred at 40° C. for 18 h. The reaction mixture was cooled to 0° C. Sodium borohydride (1.135 g, 30 mmol) was added portionwise over 10 min. After complete addition the reaction mixture was stirred at room temperature for 2 h. Saturated ammonium chloride solution (50 mL) was added slowly. The mixture was stirred at room temperature for 10 min. The mixture was extracted with EtOAc (3×25 mL). The combined extracts were washed with brine (30 mL), dried and evaporated. The residue was chromatographed [0-60% EtOAc/cyclohexane] to give the title compound as a yellow oil (1.36 g, 7.00 mmol, 70% yield). LCMS (formic A): Rt=0.80 min, MH⁺=195.

### Description 58

### Methyl 4-(5-hydroxypentyl)benzoate (D58)

Palladium (II) acetate (112 mg, 5 mol %) was added to a stirred mixture of methyl 4-iodobenzoate (2.62 g, 10.0 mmol), pent-4-en-1-ol (1.723 g, 2.039 mL, 20 mmol), sodium bicarbonate (2.52 g, 30.0 mmol) and benzyl triethylammonium chloride (2.278 g, 10 mmol) in dry MeCN (40 mL) under nitrogen. The reaction mixture was stirred at 40° C. for 18 h. The reaction mixture was cooled to 0° C. Sodium borohydride (1.135 g, 30 mmol) was added portionwise over 10 min. After complete addition the reaction mixture was stirred at room temperature for 2 h. Saturated ammonium chloride solution (50 mL) was added slowly. The mixture was stirred at room temperature for 10 min. The mixture was extracted with EtOAc (3×25 mL). The combined extracts were washed with brine (30 mL), dried and evaporated. The residue was chromatographed [0-60% EtOAc/cyclohexane] to give the title compound as a colourless oil (1.69 g, 7.60 mmol, 76% yield). LCMS (high pH A): Rt=1.01 min, poor ionisation.

### Description 59

### Methyl 4-(6-hydroxyhex-1-yn-1-yl)benzoate (D59)

A solution of a mixture of methyl 4-iodobenzoate (3 g, 11.45 mmol), hex-5-yn-1-ol (1.515 mL, 13.74 mmol)) and triethylamine (4.79 mL, 34.3 mmol) in anhydrous THF (20 mL) was degassed by the alternate application of nitrogen and vacuum repeated 4 times and then left under an atmosphere of nitrogen. The mixture was treated with bis(triphenylphosphine)palladium(II) chloride (0.241 g, 0.343 mmol) and copper(I) iodide (0.131 g, 0.687 mmol). This was then stirred at ambient temperature for a further 2 h. The mixture was treated with aqueous ammonium chloride (10%, 50 mL) and extracted with methyl tert-butyl ether (3×40 mL). The combined organics were washed with brine (40 mL) and then dried (MgSO₄), filtered and evaporated to dryness. The product was purified by chromatography on silica using a gradient elution from 0% to 50% EtOAc in DCM to give the title compound (2.43 g, 10.46 mmol, 91% yield). LCMS (formic A): Rt=0.99 min, MH⁺=233.

### Description 60

### Methyl 4-(6-hydroxyhexyl)benzoate (D60)

A solution of methyl 4-(6-hydroxyhex-1-yn-1-yl)benzoate (may be prepared as described in Description 59; 2.5 g, 10.76 mmol) in MeOH (20 mL) was added to palladium on carbon (10% degussa type) (100 mg, 0.940 mmol) in a nitrogen atmosphere then stirred under an atmosphere of hydrogen for 6 h. The mixture was filtered through a bed of florisil and the filtrate was evaporated to dryness to give the title compound (2.1 g, 8.89 mmol, 83% yield). LCMS (formic A): Rt=1.04 min, MH⁺=237 (weak), M-CH₃⁺=223.

### Description 61

### 6-(4-(hydroxymethyl)piperidin-1-yl)pyridazine-3-carboxylic acid (D61)

A solution of methyl 6-(4-(hydroxymethyl)piperidin-1-yl)pyridazine-3-carboxylate (may be prepared as described in Description 52; 17 g, 67.7 mmol) in MeOH (50 mL) was treated with 2 M aqueous sodium hydroxide (80 mL, 160 mmol), and stirred at 60° C. for 2 h. The mixture was evaporated to dryness and treated with water (50 mL). The pH of the mixture was adjusted to 4 with the addition of concentrated aqueous HCl, then the mixture was allowed to stand at room temperature over the weekend, giving a dense suspension. The solid was collected by filtration and washed with water, then dried in vacuo to give the title compound as a beige solid (14.4 g, 60.7 mmol, 90% yield).

LCMS (formic A): Rt=0.33 min, MH⁺=238.

### Description 62

### 5-(4-(Hydroxymethyl)piperidin-1-yl)pyrazine-2-carboxylic acid (D62)

A solution of methyl 5-(4-(hydroxymethyl)piperidin-1-yl)pyrazine-2-carboxylate (may be prepared as described in Description 51; 7.2 g, 28.7 mmol) in MeOH (50 mL) was treated with 2 M aqueous sodium hydroxide (28.7 mL, 57.3 mmol), and stirred at 60° C. for 2 h. The mixture was evaporated to dryness and treated with water (50 mL). The pH of the mixture was adjusted to 4 with the addition of 6 M aqueous HCl then cooled in an ice-water bath, and the precipitated product was filtered off, washed with minimal ice/water, then with minimum diethyl ether and dried under vacuum to afford the title compound (3.9 g, 16.44 mmol, 57% yield). LCMS (formic A): Rt=0.51 min, MH⁺=238.

### Description 63

### 2-(4-(Hydroxymethyl)piperidin-1-yl)pyrimidine-5-carboxylic acid (D63)

A solution of ethyl 2-(4-(hydroxymethyl)piperidin-1-yl)pyrimidine-5-carboxylate (may be prepared as described in Description 53; 7.1 g, 26.8 mmol) in MeOH (50 mL) was treated with 2 M aqueous sodium hydroxide (26.8 mL, 53.5 mmol), and stirred at 60° C. for 2 h. The mixture was evaporated to dryness and treated with water (30 mL). The pH of the mixture was adjusted to 4 with the addition of 6 M aqueous HCl then cooled in an ice-water bath and the precipitated product was filtered off, washed with minimal ice/water, then with minimum diethyl ether and dried under vacuum to give the title compound (2.95 g, 12.43 mmol, 46% yield). LCMS (formic A): Rt=0.59 min, MH⁺=238.

### Description 64

### 6-(4-(Hydroxymethyl)piperidin-1-yl) nicotinic acid (D64)

A solution of methyl 6-(4-(hydroxymethyl)piperidin-1-yl) nicotinate (may be prepared as described in Description 54; 5.2 g, 20.78 mmol) in MeOH (50 mL) was treated with 2 M aqueous sodium hydroxide (20.78 mL, 41.6 mmol), and stirred at 60° C. for 2 h. The mixture was evaporated to dryness and treated with water (50 mL). The pH of the mixture was adjusted to 4 with the addition of 6 M aqueous HCl then cooled in an ice-water bath, and the precipitated product was filtered off, washed with minimal ice/water, then with minimal diethyl ether and dried under vacuum to to give the title compound (4.2 g, 17.78 mmol, 86% yield). LCMS (high pH A): Rt=0.40 min, MH⁺=237

### Description 65

### 6-(4-(2-((tert-Butyldimethylsilyl)oxy)ethyl)piperidin-1-yl)pyridazine-3-carboxylic acid (D65)

A suspension of methyl 6-(4-(2-((tert-butyldimethylsilyl)oxy)ethyl)piperidin-1-yl)pyridazine-3-carboxylate (may be prepared as described in Description 56; 2.45 g, 6.45 mmol) in MeOH (20 mL) was treated with 2 M aqueous sodium hydroxide (4.84 mL, 9.68 mmol) and stirred in a stoppered vessel at room temperature for 22 h. The reaction mixture was evaporated in vacuo and water (5 mL) added. The mixture was carefully acidified with 2 M HCl (aq) to PH˜6-7. The aqueous was extracted with EtOAc (2×25 mL), the extracts combined, and passed through a hydrophobic frit. The filtrate was evaporated in vacuo to give the title compound as an off white solid (1.92 g, 5.25 mmol). LCMS (high pH A): Rt=0.95 min, MH⁺=366.

### Description 66

### Sodium 6-(1,4-dioxa-8-azaspiro[4.5]decan-8-yl)pyridazine-3-carboxylate (D66)

A mixture of methyl 6-(1,4-dioxa-8-azaspiro[4.5]decan-8-yl)pyridazine-3-carboxylate (may be prepared as described in Description 55; 2.2 g, 7.88 mmol) and aqueous 2 M sodium hydroxide solution (11.82 mL, 23.63 mmol) was diluted in MeOH (15 mL) and THF (15 mL) and stirred in stoppered vessel at 45° C. for 2 h. The reaction mixture was then evaporated using a rotary evaporator and the resulting gum was then acidified with aqueous 2 M HCl solution to pH 5. This solution was then extracted with DCM (150 mL) and the organic layer then passed through a hydrophobic frit. The filtrate was then evaporated to dryness using a rotary evaporator to give 6-(1,4-dioxa-8-azaspiro[4.5]decan-8-yl)pyridazine-3-carboxylic acid as a light yellow powder (450 mg, 1.696 mmol). The aqueous layer was then evaporated to dryness using a rotary evaporator to give the title compound as an off white powder (2.0 g, 6.94 mmol) which was used without purification in the next step. LCMS (formic A): Rt=0.43 min, MH⁺=266.

### Description 67

### 4-(3-Hydroxypropyl)benzoic acid (D67)

2 M Sodium hydroxide (5 mL, 10 mmol) was added to a stirred solution of methyl 4-(3-hydroxypropyl)benzoate (may be prepared as described in Description 57; 1.36 g, 7.00 mmol) in ethanol (5 mL). The reaction mixture was stirred at room temperature for 18 h. The ethanol was evaporated. The residue was diluted with water (20 mL). The mixture was acidified by the dropwise addition of 5M hydrochloric acid. The mixture was extracted with EtOAc (3×25 mL). The combined extracts were dried and evaporated to give the title compound as a yellow solid (1.20 g, 6.66 mmol, 95% yield). LCMS (formic A): Rt=0.60 min, weak ionisation.

### Description 68

### 4-(5-Hydroxypentyl)benzoic acid (D68)

2 M sodium hydroxide (10 mL, 20 mmol) as added to a stirred solution of methyl 4-(5-hydroxypentyl)benzoate (may be prepared as described in Description 58; 1.60 g, 7.20 mmol) in ethanol (10 mL). The reaction mixture was stirred at room temperature for 2 h, then allowed to stand for 18 h. The ethanol was evaporated. The residue was diluted with water (15 mL). The mixture was acidified by the dropwise addition of 5M hydrochloric acid. The mixture was extracted with EtOAc (3×10 mL). The combined extracts were dried and evaporated to give the title compound as a colourless solid (1.31 g, 6.29 mmol, 87% yield). LCMS (formic A): Rt=0.78 min, MH⁺=209.

### Description 69

### 4-(6-Hydroxyhexyl)benzoic acid (D69)

A solution of methyl 4-(6-hydroxyhexyl)benzoate (may be prepared as described in Description 60; 2 g, 8.46 mmol) in Ethanol (10 mL) was treated with aqueous sodium hydroxide (2 M) (6.35 mL, 12.70 mmol) and the mixture was stirred at 50° C. for 2 h. The mixture was reduced in volume by a half and then treated with water (10 m). The solution was treated dropwise with aqueous hydrochloric acid (2 M) until pH4 was achieved and a white suspension was produced. The suspension was filtered off, washed with water and dried under vacuum to give the title compound as a white solid (1.52 g, 6.84 mmol, 81% yield). LCMS (formic A): Rt=0.88 min, poor ionisation.

### Description 70

### N-((1r,4r)-4-(3-Chloro-4-cyanophenoxy)cyclohexyl)-6-(4-(hydroxymethyl)piperidin-1-yl)pyridazine-3-carboxamide (D70)

4-(((1r,4r)-4-Aminocyclohexyl)oxy)-2-chlorobenzonitrile (may be prepared as described in Description 46; 2.8 g, 11.17 mmol), 6-(4-(hydroxymethyl)piperidin-1-yl)pyridazine-3-carboxylic acid (may be prepared as described in Description 61; 2.8 g, 11.80 mmol), HATU (5.52 g, 14.52 mmol) and DIPEA (5.85 mL, 33.5 mmol) were stirred in DCM (30 mL) at room temperature for 4 h, then the mixture was washed with water (40 mL) and the organic layer dried and evaporated in vacuo to give a brown gum. The crude was dissolved in DCM and loaded onto a 40 g silica column, then eluted with 0-100% (25% ethanol/EtOAc 1% NH₄OH)/cyclohexane and product-containing fractions were evaporated in vacuo to give the title compound as a colourless solid (3.8 g, 8.09 mmol, 72% yield). LCMS (formic A): Rt=1.08 min, MH⁺=470, 472

### Description 71

### N-((1r,4r)-4-(3-Chloro-4-cyanophenoxy)cyclohexyl)-6-(4-(hydroxymethyl)piperidin-1-yl)nicotinamide (D71)

4-(((1r,4r)-4-Aminocyclohexyl)oxy)-2-chlorobenzonitrile (may be prepared as described in Description 46; 1 g, 3.99 mmol), 6-(4-(hydroxymethyl)piperidin-1-yl) nicotinic acid (may be prepared as described in Description 64; 0.942 g, 3.99 mmol), HATU (1.971 g, 5.18 mmol) and DIPEA (2.090 mL, 11.97 mmol) were stirred in DCM (30 mL) at room temperature for 4 h, then the mixture was washed with water (40 mL) and the organic layer dried and evaporated in vacuo to give the title compound as a pale yellow gum (3.4 g) which contained residual DCM and TMU. Used in the next step without purification. LCMS (high pH A): Rt=1.07 min, MH⁺=469, 471.

### Description 72

### N-((1r,3r)-3-(3-Chloro-4-cyanophenoxy)-2,2,4,4-tetramethylcyclobutyl)-6-(4-(hydroxymethyl)piperidin-1-yl)pyridazine-3-carboxamide (D72)

4-((1r,3r)-3-Amino-2,2,4,4-tetramethylcyclobutoxy)-2-chlorobenzonitrile (may be prepared as described in Description 49; 1.02 g, 3.66 mmol), 6-(4-(hydroxymethyl)piperidin-1-yl)pyridazine-3-carboxylic acid (may be prepared as described in Description 61; 0.868 g, 3.66 mmol), HATU (1.809 g, 4.76 mmol) and DIPEA (1.917 mL, 10.98 mmol) were stirred in DCM (30 mL) at room temperature for 4 h, then the mixture was washed with water (40 mL) and the organic layer dried and evaporated in vacuo to give a brown gum The crude was dissolved in DCM and loaded onto a 40 g silica column, then eluted with 0-100% (25% ethanol/EtOAc 1% NH₄OH)/cyclohexane and product-containing fractions were evaporated in vacuo to give the title compound (1.42 g, 2.85 mmol, 78% yield) as a colourless solid. LCMS (formic A): Rt=1.23 min, MH⁺=498, 500.

### Description 73

### N-((1r,3r)-3-(3-Chloro-4-cyanophenoxy)-2,2,4,4-tetramethylcyclobutyl)-5-(4-(hydroxymethyl)piperidin-1-yl)pyrazine-2-carboxamide (D73)

4-((1r,3r)-3-Amino-2,2,4,4-tetramethylcyclobutoxy)-2-chlorobenzonitrile (may be prepared as described in Description 49; 0.59 g, 2.116 mmol), 5-(4-(hydroxymethyl)piperidin-1-yl)pyrazine-2-carboxylic acid (may be prepared as described in Description 62; 0.502 g, 2.116 mmol), HATU (1.046 g, 2.75 mmol) and DIPEA (1.109 mL, 6.35 mmol) were stirred in DCM (30 mL) at room temperature for 4 h, then the mixture was washed with water (40 mL) and the organic layer dried and evaporated in vacuo to give the title compound as a brown gum. The compound was carried through to the next step without purification. LCMS (high pH A): Rt=1.29 min, MH⁺=496, 498.

### Description 74

### N-((1r,3r)-3-(3-chloro-4-cyanophenoxy)-2,2,4,4-tetramethylcyclobutyl)-2-(4-(hydroxymethyl)piperidin-1-yl)pyrimidine-5-carboxamide (D74)

A mixture of 2-(4-(hydroxymethyl)piperidin-1-yl)pyrimidine-5-carboxylic acid (may be prepared as described in Description 63; 400 mg, 1.686 mmol) and HATU (800 mg, 2.104 mmol) in anhydrous DMF (4 mL) was treated with DIPEA (0.73 mL, 4.18 mmol) and the suspension stirred in a stoppered vessel at room temperature for 20 min (a solution formed over this time). 4-((1r,3r)-3-Amino-2,2,4,4-tetramethylcyclobutoxy)-2-chlorobenzonitrile (may be prepared as described in Description 49; 470 mg, 1.686 mmol) was added and the mixture stirred in a stoppered vessel at room temperature for 1 h (a suspension formed over this time). The reaction mixture was diluted water (25 mL) and filtered. The solid was washed sequentially with water (100 mL) and diethylether (25 mL) and dried in a vacuum oven to give the title compound as an off white solid (648 mg, 1.301 mmol). The compound was carried through to the next step without purification. LCMS (high pH A): Rt=1.24 min, MH⁺=498, 500.

### Description 75

### N-((1r,3r)-3-(3-Chloro-4-cyanophenoxy)-2,2,4,4-tetramethylcyclobutyl)-6-(4-(hydroxymethyl)piperidin-1-yl)nicotinamide (D75)

4-((1r,3r)-3-Amino-2,2,4,4-tetramethylcyclobutoxy)-2-chlorobenzonitrile (may be prepared as Description 49; 0.5 g, 1.794 mmol), 6-(4-(hydroxymethyl)piperidin-1-yl) nicotinic acid (may be prepared as described in Description 64; 0.424 g, 1.794 mmol), HATU (0.887 g, 2.332 mmol) and DIPEA (0.940 mL, 5.38 mmol) were stirred in DCM (30 mL) at room temperature for 4 h, giving a dense suspension. This was filtered and the solid washed with DCM (10 mL) and dried to give the title compound as a colourless solid (0.72 g, 1.449 mmol, 81% yield). The compound was carried through to the next step without purification. LCMS (formic A): Rt=1.02 min, MH⁺=497, 499.

### Description 76

### N-((1r,4r)-4-(4-Cyano-3-(trifluoromethyl)phenoxy)cyclohexyl)-6-(4-(hydroxymethyl)piperidin-1-yl)pyridazine-3-carboxamide (D76)

To a suspension of 6-(4-(hydroxymethyl)piperidin-1-yl)pyridazine-3-carboxylic acid (may be prepared as described in Description 61; 0.814 g, 3.43 mmol), 4-(((1r,4r)-4-aminocyclohexyl)oxy)-2-(trifluoromethyl)benzonitrile hydrochloride (may be prepared as described in Description 48; 1 g, 3.12 mmol), PyBOP (1.947 g, 3.74 mmol) and ethyl (E)-2-cyano-2-(hydroxyimino)acetate (0.532 g, 3.74 mmol) in DMF (37.1 mL) was added N-methylmorpholine (1.028 mL, 9.35 mmol), the vessel was sealed, then the reaction mixture was stirred at room temperature for 57 h. Additional PyBOP (0.649 g, 1.25 mmol) and N-methylmorpholine (0.34 mL, 3.12 mmol) were added, then the reaction mixture was stirred at room temperature for 4 h. The reaction mixture was diluted with EtOAc (80 mL) and washed sequentially with water (70 mL), 5% LiCl (aq) (70 mL) and brine (70 mL). The water layer was further extracted with EtOAc (3×80 mL). The organic layers were combined and washed with brine (100 mL), then the organic layer was passed through a hydrophobic frit, and the solvent removed in vacuo. The residue was purified by normal phase column chromatography (0-25% 3:1 EtOAc in ethanol in TBME, 120 g silica, 10 column volumes) to give the title compound as a yellow oil (1.547 g, 2.504 mmol, 77% yield). LCMS (formic A): Rt=1.06 min, MH⁺=504.

### Description 77

### N-((1r,4r)-4-(3-chloro-4-cyanophenoxy)cyclohexyl)-5-(4-(hydroxymethyl)piperidin-1-yl)pyrazine-2-carboxamide (D77)

4-(((1r,4r)-4-Aminocyclohexyl)oxy)-2-chlorobenzonitrile (may be prepared as described in Description 46; 0.5 g, 1.994 mmol), 5-(4-(hydroxymethyl)piperidin-1-yl)pyrazine-2-carboxylic acid (may be prepared as described in Description 62; 0.473 g, 1.994 mmol), HATU (0.986 g, 2.59 mmol) and DIPEA (1.045 mL, 5.98 mmol) were stirred in DCM (30 mL) at room temperature for 4 h, then the mixture was washed with water (40 mL) and the organic layer dried and evaporated in vacuo to give the title compound as a brown gum, which contained about 1 equivalent of tetramethyl urea. Used in the next step without purification. LCMS (high pH A): Rt=1.12 min, MH⁺=470, 472.

### Description 78

### N-((1r,4r)-4-(4-Cyano-3-(trifluoromethyl)phenoxy)cyclohexyl)-2-(4-(hydroxymethyl)piperidin-1-yl)pyrimidine-5-carboxamide (D78)

A mixture of 2-(4-(hydroxymethyl)piperidin-1-yl)pyrimidine-5-carboxylic acid (may be prepared as described in Description 63; 400 mg, 1.686 mmol) and HATU (800 mg, 2.104 mmol) in anhydrous DMF (4 mL) was treated with DIPEA (1.03 mL), and the suspension stirred in a stoppered vessel at room temperature for 20 min (a solution formed over this time). 4-(((1r,4r)-4-Aminocyclohexyl)oxy)-2-(trifluoromethyl)benzonitrile hydrochloride (may be prepared as described in Description 48; 541 mg, 1.686 mmol) was added, and the mixture stirred in a stoppered vessel at room temperature for 1 h. The reaction mixture was diluted with EtOAc (25 mL) and washed sequentially with water (25 mL), 5% LiCl (aq) (25 mL) and brine (25 mL). The organic layer was passed through a hydrophobic frit and the filtrate evaporated in vacuo. The resulting solid was triturated with petroleum ether (2×10 mL) and dried in a vacuum oven to give the title compound as a light brown solid (922 mg, 1.831 mmol). LCMS (high pH A): Rt=1.12 min, MH⁺=504.

### Description 79

### N-((1r,4r)-4-(4-Cyano-3-(trifluoromethyl)phenoxy)cyclohexyl)-5-(4-(hydroxymethyl)piperidin-1-yl)pyrazine-2-carboxamide (D79)

4-(((1r,4r)-4-Aminocyclohexyl)oxy)-2-(trifluoromethyl)benzonitrile hydrochloride (may be prepared as described in Description 48; 0.52 g, 1.621 mmol), 5-(4-(hydroxymethyl)piperidin-1-yl)pyrazine-2-carboxylic acid (may be prepared as described in Description 62; 0.385 g, 1.621 mmol), HATU (0.801 g, 2.108 mmol) and DIPEA (0.849 mL, 4.86 mmol) were stirred in DCM (30 mL) at room temperature for 4 h, then the mixture was washed with water (40 mL) and the organic layer dried and evaporated in vacuo to give the title compound a brown gum. Carried through to the next step without further purification. LCMS (high pH A): Rt=1.15 min, MH⁺=504.

### Description 80

### N-((1r,4r)-4-(4-Cyano-3-(trifluoromethyl)phenoxy)cyclohexyl)-6-(4-(hydroxymethyl)piperidin-1-yl)nicotinamide (D80)

4-(((1r,4r)-4-Aminocyclohexyl)oxy)-2-(trifluoromethyl)benzonitrile hydrochloride (may be prepared as described in Description 48; 0.52 g, 1.621 mmol), 6-(4-(hydroxymethyl)piperidin-1-yl) nicotinic acid (may be prepared as described in Description 64; 0.383 g, 1.621 mmol), HATU (0.801 g, 2.108 mmol) and DIPEA (0.849 mL, 4.86 mmol) were stirred in DCM (30 mL) at room temperature for 4 h, then the mixture was washed with water (40 mL) and the organic layer dried and evaporated in vacuo to give a brown gum The crude was dissolved in DCM and loaded onto a 40 g silica column, then eluted with 0-100% EtOAc/cyclohexane and product-containing fractions evaporated in vacuo to give the title compound (750 mg, 1.492 mmol, 92% yield) as a colourless foam. LCMS (high pH A): Rt=1.10 min, MH⁺=503.

### Description 81

### N-((1r,4r)-4-(3-Chloro-4-cyanophenoxy)cyclohexyl)-2-(4-(hydroxymethyl)piperidin-1-yl)pyrimidine-5-carboxamide (D81)

A mixture of 2-(4-(hydroxymethyl)piperidin-1-yl)pyrimidine-5-carboxylic acid (may be prepared as described in Description 63; 400 mg, 1.686 mmol) and HATU (800 mg, 2.104 mmol) in anhydrous DMF (4 mL) was treated with DIPEA (0.73 mL, 4.18 mmol), and the suspension was stirred in a stoppered vessel at room temperature for 20 min (a solution formed over this time). 4-(((1r,4r)-4-Aminocyclohexyl)oxy)-2-chlorobenzonitrile (may be prepared as described in Description 46; 423 mg, 1.686 mmol) was added, and the mixture was stirred in a stoppered vessel at room temperature for 1 h. The reaction mixture was diluted with EtOAc (25 mL) and washed sequentially with water (25 mL), 5% LiCl (aq) (25 mL) and brine (25 mL). The organic layer was passed through a hydrophobic frit and the filtrate evaporated in vacuo. The resulting solid was triturated with diethylether (2×10 mL) and dried in a vacuum oven to give the title compound as a light brown solid (879 mg, 1.870 mmol). LCMS (high pH A): Rt=1.08 min, MH⁺=470, 472

### Description 82

### N-((1r,4r)-4-(3-Chloro-4-cyanophenoxy)cyclohexyl)-4-(hydroxymethyl)benzamide (D82)

A mixture of 4-(hydroxymethyl)benzoic acid (2 g, 13.15 mmol), 4-(((1r,4r)-4-aminocyclohexyl)oxy)-2-chlorobenzonitrile (may be prepared as described in Description 46; 3.30 g, 13.15 mmol), DIPEA (6.89 mL, 39.4 mmol) and HATU (6.00 g, 15.77 mmol) in DCM (50 mL) was stirred at room temperature for 1.5 h. The mixture was washed with water, dried and evaporated in vacuo. The crude was dissolved in EtOAc (50 mL, reflux), then allowed to cool to room temperature and the resulting solid collected by filtration to give the title compound as a colourless solid (4.5 g, 11.69 mmol, 89% yield). LCMS (high pH A): Rt=1.04 min, MH⁺=385, 387.

### Description 83

### N-((1r,4r)-4-(3-Chloro-4-cyanophenoxy)cyclohexyl)-4-(3-hydroxypropyl)benzamide (D83)

A mixture of 4-(3-hydroxypropyl)benzoic acid (may be prepared as described in Description 67; 500 mg, 2.77 mmol), 4-(((1r,4r)-4-aminocyclohexyl)oxy)-2-chlorobenzonitrile (may be prepared as described in Description 46; 835 mg. 3.33 mmol), DIPEA (717 mg, 0.969 mL, 5.55 mmol) and HATU (1266 mg, 3.33 mmol) in DMF (5 mL) was stirred at room temperature for 1.5 h. The reaction mixture was partitioned between EtOAc (25 mL) and saturated sodium bicarbonate solution (25 mL). The aqueous phase was separated. The organic phase was washed with 2 M hydrochloric acid (10 mL), water (10 mL) and brine (10 mL). The organic phase was dried and evaporated. The residue was chromatographed [0-10% ethanol/DCM] to give the title compound as an off white solid (895 mg, 2.168 mmol, 78% yield). LCMS (high pH A): Rt=1.07 min, MH⁺=413, 415.

### Description 84

### N-((1r,4r)-4-(3-Chloro-4-cyanophenoxy)cyclohexyl)-4-(5-hydroxypentyl)benzamide (D84)

A mixture of 4-(5-hydroxypentyl)benzoic acid (may be prepared as described in Description 68; 500 mg, 2.401 mmol), 4-(((1r,4r)-4-aminocyclohexyl)oxy)-2-chlorobenzonitrile (may be prepared as described in Description 46; 722 mg. 2.88 mmol), DIPEA (621 mg, 0.839 mL, 4.80 mmol) and HATU (1095 mg, 2.88 mmol) in DMF (5 mL) was stirred at room temperature for 1.5 h. The reaction mixture was partitioned between EtOAc (25 mL) and saturated sodium bicarbonate solution (20 mL). The aqueous phase was separated. The organic phase was washed with 2 M hydrochloric acid (10 mL), water (10 mL) and brine (10 mL). The organic phase was dried and evaporated. The residue was chromatographed [0-10% ethanol/DCM] to give the title compound as an off white solid (0.97 g, 2.200 mmol, 92% yield). LCMS (formic A): Rt=1.17 min, MH⁺=441, 443.

### Description 85

### N-((1r,4r)-4-(3-Chloro-4-cyanophenoxy)cyclohexyl)-4-(6-hydroxyhexyl)benzamide (D85)

A mixture of 4-(6-hydroxyhexyl)benzoic acid (500 mg, 2.249 mmol), 4-(((1r,4r)-4-aminocyclohexyl)oxy)-2-chlorobenzonitrile hydrochloride (may be prepared as described in Description 45; 775 mg, 2.70 mmol), HATU (1026 mg, 2.70 mmol), and DIPEA (1.179 mL, 6.75 mmol) in DMF (7 mL) was stirred at room temperature for 2 h. The reaction mixture was partitioned between EtOAc (15 mL) and saturated sodium bicarbonate solution (15 mL). The aqueous phase was separated. The organic phase was washed with 2 M hydrochloric acid (10 mL), water (10 mL) and brine (10 mL). The organic phase was dried and evaporated. The residue was purified by normal phase column chromatography (30-70% EtOAc in cyclohexane, 80 g silica, 12 column volumes) to afford the title compound as a white solid (863 mg, 1.897 mmol, 84% yield). LCMS (formic A): Rt=1.22 min, MH⁺=455.

### Description 86

### N-((1r,4r)-4-(3-Chloro-4-cyanophenoxy)cyclohexyl)-6-(4-(2-hydroxyethyl)piperidin-1-yl)pyridazine-3-carboxamide (D86)

A suspension of 6-(4-(2-((tert-butyldimethylsilyl)oxy)ethyl)piperidin-1-yl)pyridazine-3-carboxylic acid (may be prepared as described in Description 65; 969 mg, 2.65 mmol), ethyl (hydroxyimino) cyanoacetate (436 mg, 3.07 mmol), 4-(((1r,4r)-4-aminocyclohexyl)oxy)-2-chlorobenzonitrile hydrochloride (may be prepared as described in Description 45; 720 mg, 2.507 mmol) and PyBOP (1610 mg, 3.09 mmol) in anhydrous DMF (15 mL) was treated with N-methylmorpholine (0.83 mL, 7.55 mmol), and the mixture was allowed to stir at room temperature in a stoppered vessel for 22 h. The mixture was diluted with EtOAc (30 mL) and washed sequentially with water (25 mL), 5% LiCl (aq) (25 mL) and brine (25 mL). The organic layer was passed through a hydrophobic frit and the filtrate evaporated in vacuo. The resulting gum was dissolved in MeOH (15 mL) and treated with 2 M HCl (aq) (5.0 mL, 10.00 mmol). The solution was allowed to stand in a stoppered vessel at room temperature for 2 h. The mixture was basified with 2 M NaOH (aq) to ˜pH=10 and partially evaporated in vacuo. The remaining aqueous solution was extracted with EtOAc (25 mL) and the organic layer washed sequentially with sat. NaHCO₃ (aq) (25 mL) and brine (25 mL). The organic layer was passed through a hydrophobic frit and the filtrate evaporated in vacuo. The residue was loaded in DCM (5 mL) and purified on a 80 g silica cartridge using a gradient of 0-75% EtOAc:ethanol (3:1) in TBME over 12 column volumes. The appropriate fractions were combined and the solvent evaporated in vacuo. The solid was dried under high vacuum for 16 h to give the title compound as an off white solid (835 mg, 1.725 mmol). LCMS (formic A): Rt=1.03 min, MH⁺=484, 486.

### Description 87

### N-((1r,4r)-4-(3-Chloro-4-cyanophenoxy)cyclohexyl)-4-(3-hydroxypiperidin-1-yl)benzamide (D87)

A mixture of 4-(3-hydroxypiperidin-1-yl)benzoic acid (100 mg, 0.452 mmol), 4-(((1r,4r)-4-aminocyclohexyl)oxy)-2-chlorobenzonitrile trifluoroacetic acid salt (may be prepared as described in Description 47; 198 mg, 0.542 mmol), DIPEA (117 mg, 0.158 mL, 0.904 mmol) and HATU (206 mg, 0.542 mmol) in DMF (3 mL) was stirred at room temperature for 1.5 h. The reaction mixture was partitioned between EtOAc (15 mL) and saturated sodium bicarbonate solution (15 mL). The aqueous phase was separated. The organic phase was washed with water (10 mL) and brine (10 mL). The organic phase was dried and evaporated. The residue was chromatographed [0-10% ethanol/EtOAc] to give the title compound as an off white solid (155 mg, 0.341 mmol, 76% yield). LCMS (high pH A): Rt=1.10 min, MH⁺=454, 456.

### Description 88

### N-((1r,3r)-3-(4-Cyano-3-(trifluoromethyl)phenoxy)-2,2,4,4-tetramethylcyclobutyl)-4-(6-hydroxyhexyl)benzamide (D88)

A mixture of 4-((1r,3r)-3-amino-2,2,4,4-tetramethylcyclobutoxy)-2-(trifluoromethyl)benzonitrile hydrochloride (may be prepared as described in Description 50; 300 mg, 0.860 mmol), 4-(6-hydroxyhexyl)benzoic acid (191 mg, 0.860 mmol) and triethylamine (0.480 mL, 3.44 mmol) in DCM (10 mL) was treated with HATU (425 mg, 1.118 mmol) and stirred at ambient temperature for 30 min. The mixture was treated with DCM (10 mL) and saturated aqueous sodium bicarbonate (20 mL). The aqueous phase was extracted with further DCM (10 mL) and the combined organics were evaporated to dryness. The product was purified by chromatography on silica using a gradient elution from 0% to 100% EtOAc in cyclohexane to give the title compound (390 mg, 0.755 mmol, 88% yield). LCMS (formic A): Rt=1.38 min, MH⁺=517.

### Description 89

### N-((1r,4r)-4-(3-Chloro-4-cyanophenoxy)cyclohexyl)-6-(1,4-dioxa-8-azaspiro[4.5]decan-8-yl)pyridazine-3-carboxamide (D89)

A mixture of sodium 6-(1,4-dioxa-8-azaspiro[4.5]decan-8-yl)pyridazine-3-carboxylate (may be prepared as described in Description 66; 1.45 g, 5.05 mmol), PyBop (3.15 g, 6.06 mmol), OxymaPure (0.85 g, 5.98 mmol) and DIPEA (3.10 mL, 17.75 mmol) were suspended in DMF (35 mL) and allowed to stir at room temperature for 30 min. 4-(((1r,4r)-4-Aminocyclohexyl)oxy)-2-chlorobenzonitrile (may be prepared as described in Description 46; 1.75 g, 6.09 mmol) was then added to the mixture before being allowed to stir at room temperature for 16 h. The mixture was diluted with water (30 mL), extracted with EtOAc (100 mL) and washed with 5% aq LiCl solution (20 mL). The organic layer was then passed through a hydrophobic frit and the filtrate evaporated to dryness. The resulting residue was then loaded in DCM (10 mL) and purified on a 40 g silica cartridge using a gradient of 0-60% EtOAc:ethanol (3:1) in TBME over 14 column volumes. The appropriate fractions were combined and the solvent removed by rotary evaporation to give the title compound as an orange solid (700 mg, 1.406 mmol). LCMS (formic A): Rt=1.17 min, MH⁺=498, 500.

### Description 90

### 6-Chloro-N-((1r,4r)-4-(3-chloro-4-cyanophenoxy)cyclohexyl)pyridazine-3-carboxamide (D90)

A mixture of 6-chloropyridazine-3-carboxylic acid (0.5 g, 3.15 mmol), 4-(((1r,4r)-4-aminocyclohexyl)oxy)-2-chlorobenzonitrile trifluoroacetic acid salt (may be prepared as described in Description 47; 1.380 g. 3.78 mmol), DIPEA (815 mg, 1.102 mL, 6.31 mmol) and HATU (1.439 g, 3.78 mmol) in DMF (10 mL) was stirred at room temperature for 1.5 h. The reaction mixture was partitioned between EtOAc (50 mL) and saturated sodium bicarbonate solution (50 mL). The mixture was filtered through Celite. The aqueous phase was separated. The organic phase was washed with water (10 mL) and brine (10 mL). The organic phase was dried and evaporated. The residue was chromatographed [30-75% EtOAc/cyclohexane] to give the title compound (640 mg, 1.636 mmol, 52% yield), as an off white solid. LCMS (high pH A): Rt=1.17 min, MH⁺=389, 391.

### Description 91

### N-((1r,4r)-4-(3-Chloro-4-cyanophenoxy)cyclohexyl)-6-(3-(hydroxymethyl)piperidin-1-yl)pyridazine-3-carboxamide (D91)

6-Chloro-N-((1r,4r)-4-(3-chloro-4-cyanophenoxy)cyclohexyl)pyridazine-3-carboxamide (may be prepared as described in Description 90; 200 mg, 0.511 mmol), piperidin-3-ylmethanol (70.6 mg, 0.613 mmol) and DIPEA (0.107 mL, 0.613 mmol) were added to a microwave vial to which was added DMSO (1 mL), and the mixture heated at 70° C. for 2 h, then the mixture was diluted with water and extracted with EtOAc. The organics were washed with water, dried and evaporated in vacuo to give a pale yellow gum. This was dissolved in DCM and loaded onto a 24 g silica column, then eluted with 0-100% (25% ethanol/EtOAc 1% NH₄OH)/cyclohexane and product-containing fractions evaporated in vacuo to give the title compound (232 mg, 0.494 mmol, 97% yield) as a pale yellow gum. LCMS (high pH A): Rt=1.10 min, MH⁺=470, 472.

### Description 92

### N-((1r,3r)-3-(3-Chloro-4-cyanophenoxy)-2,2,4,4-tetramethylcyclobutyl)-5-(4-formylpiperidin-1-yl)pyrazine-2-carboxamide (D92)

N-((1r,3r)-3-(3-Chloro-4-cyanophenoxy)-2,2,4,4-tetramethylcyclobutyl)-5-(4-(hydroxymethyl)piperidin-1-yl)pyrazine-2-carboxamide (may be prepared as described in Description 73; 1.52 g, 2.136 mmol) was dissolved in DCM (20 mL) and cooled in an ice bath, and Dess-Martin periodinane (1.2 g, 2.83 mmol) was added, then the mixture stirred at room temperature for 2 h, then evaporated in vacuo to give a yellow gum. The crude was suspended in DCM, filtered and the filtrate purified by chromatography on a 12 g silica column eluting with 0-100% EtOAc/cyclohexane. Product-containing fractions were evaporated in vacuo to give the title compound as a pale yellow solid (0.76 g, 1.532 mmol, 72% yield). LCMS (formic A): Rt=1.35 min, M+MeOH+528, 530

### Description 93

### N-((1r,3r)-3-(3-Chloro-4-cyanophenoxy)-2,2,4,4-tetramethylcyclobutyl)-2-(4-formylpiperidin-1-yl)pyrimidine-5-carboxamide (D93)

N-((1r,3r)-3-(3-Chloro-4-cyanophenoxy)-2,2,4,4-tetramethylcyclobutyl)-2-(4-(hydroxymethyl)piperidin-1-yl)pyrimidine-5-carboxamide (may be prepared as described in Description 74; 648 mg, 1.301 mmol) was dissolved in DCM (8 mL) and Dess-Martin periodinane (1.2 g, 2.83 mmol) was added, then the mixture stirred at room temperature for 4 h, suspended in DCM (10 mL) and filtered through Celite. The filtrate was purified on a 40 g silica cartridge using a gradient of 0-100% EtOAc in cyclohexane over 12 column volumes. The appropriate fractions were combined and the solvent evaporated in vacuo. The residue was suspended in 10% MeOH in DCM (20 mL) and washed with 0.5 M NaOH (aq) (2×10 mL) (4 mL of brine was added to aid separation). The organic layer was passed through a hydrophobic frit, evaporated under a stream of nitrogen and dried in a vacuum oven to give the title compound as an off white solid (132 mg, 0.266 mmol). LCMS (formic A): Rt=1.33 min, MH⁺=496, 498.

### Description 94

### N-((1r,4r)-4-(3-Chloro-4-cyanophenoxy)cyclohexyl)-6-(4-formylpiperidin-1-yl)nicotinamide (D94)

N-((1r,4r)-4-(3-Chloro-4-cyanophenoxy)cyclohexyl)-6-(4-(hydroxymethyl)piperidin-1-yl)nicotinamide (may be prepared as described in Description 71; 3.4 g, 4.35 mmol) was dissolved in DCM (20 mL) and cooled in an ice bath, and Des-Martin iodinane (2.398 g, 5.65 mmol) was added, then the mixture stirred at room temperature for 2 h, then evaporated in vacuo to give a colourless gum/solid. The crude was suspended in DCM, filtered and the filtrate purified by chromatography on a 40 g silica column eluting with 0-100% EtOAc/cyclohexane. Product-containing fractions were evaporated in vacuo to give the title compound (1.72 g, 3.68 mmol, 85% yield), containing ˜0.5 eq TMU from the earlier HATU step. LCMS (high pH A): Rt=1.11 min, MH⁺=467, 469.

### Description 95

### N-((1r,4r)-4-(3-Chloro-4-cyanophenoxy)cyclohexyl)-6-(4-formylpiperidin-1-yl)pyridazine-3-carboxamide (D95)

N-((1r,4r)-4-(3-chloro-4-cyanophenoxy)cyclohexyl)-6-(4-(hydroxymethyl)piperidin-1-yl)pyridazine-3-carboxamide (may be prepared as described in Description 70; 3.8 g, 8.09 mmol) was dissolved in DCM (20 mL) and cooled in an ice bath and Dess-Martin periodinane (5.14 g, 12.13 mmol) was added, then the mixture stirred at room temperature for 2 h, then evaporated in vacuo to give a gummy solid. The crude was suspended in DCM and loaded onto a 24 g silica column, then eluted with 0-100% EtOAc/cyclohexane and product-containing fractions evaporated in vacuo to give the title compound (3.01 g, 6.43 mmol, 80% yield) as a colourless foam. LCMS (formic A): Rt=1.11 min, MH⁺=468, 470.

### Description 96

### N-((1r,3r)-3-(3-Chloro-4-cyanophenoxy)-2,2,4,4-tetramethylcyclobutyl)-6-(4-formylpiperidin-1-yl)nicotinamide (D96)

N-((1r,3r)-3-(3-Chloro-4-cyanophenoxy)-2,2,4,4-tetramethylcyclobutyl)-6-(4-(hydroxymethyl)piperidin-1-yl)nicotinamide (may be prepared as described in Description 75; 720 mg, 1.449 mmol) was dissolved in DCM (20 mL) and cooled in an ice bath, and Dess-Martin periodinane (799 mg, 1.883 mmol) was added, then the mixture stirred at room temperature for 2 h, then evaporated in vacuo to give a colourless gum/solid The crude was suspended in DCM, filtered and the filtrate purified by chromatography on a 12 g silica column eluting with 0-100% EtOAc/cyclohexane. Product-containing fractions were evaporated in vacuo to give the title compound as a beige solid (410 mg, 0.828 mmol, 57% yield). LCMS (high pH A): Rt=1.35 min, MH⁺=495, 497.

### Description 97

### N-((1r,4r)-4-(4-Cyano-3-(trifluoromethyl)phenoxy)cyclohexyl)-6-(4-formylpiperidin-1-yl)pyridazine-3-carboxamide (D97)

N-((1r,4r)-4-(4-Cyano-3-(trifluoromethyl)phenoxy)cyclohexyl)-6-(4-(hydroxymethyl)piperidin-1-yl)pyridazine-3-carboxamide (may be prepared as described in Description 76; 700 mg, 1.112 mmol) was dissolved in DCM (11.5 mL) and cooled in an ice bath. Dess-Martin periodinane (708 mg, 1.668 mmol) was added, then the mixture stirred at room temperature for 4.5 h. The reaction mixture was concentrated in vacuo. The resulting yellow gum was diluted with DCM, filtered and the filtrate partially concentrated then loaded onto a cyclohexane preconditioned 12 g Redisep silica column and purified by Combiflash using a gradient of 0-100% EtOAc in cyclohexane over 15 column volumes. Fractions containing the desired product were collected, and the solvent removed in vacuo to give the title compound as a yellow gum (495 mg, 0.592 mmol, 53% yield). LCMS (high pH A): Rt=1.20 min, MH⁺=502

### Description 98

### N-((1r,4r)-4-(4-Cyano-3-(trifluoromethyl)phenoxy)cyclohexyl)-5-(4-formylpiperidin-1-yl)pyrazine-2-carboxamide (D98)

N-((1r,4r)-4-(4-Cyano-3-(trifluoromethyl)phenoxy)cyclohexyl)-5-(4-(hydroxymethyl)piperidin-1-yl)pyrazine-2-carboxamide (may be prepared as described in Description 79; 1.35 g, 1.877 mmol) was dissolved in DCM (20 mL) and cooled in an ice bath and Dess-Martin periodinane (1.2 g, 2.83 mmol) was added, then the mixture stirred at room temperature for 2 h, then evaporated in vacuo to give a yellow gum. The crude was suspended in DCM, filtered, and the filtrate purified by chromatography on a 12 g silica column eluting with 0-100% EtOAc/cyclohexane. Product-containing fractions were evaporated in vacuo to give the title compound as a pale yellow gum (0.68 g, 1.356 mmol, 72% yield). LCMS (high pH A): Rt=1.20 min, MH⁺=502.

### Description 99

### N-((1r,4r)-4-(3-Chloro-4-cyanophenoxy)cyclohexyl)-5-(4-formylpiperidin-1-yl)pyrazine-2-carboxamide (D99)

N-((1r,4r)-4-(3-Chloro-4-cyanophenoxy)cyclohexyl)-5-(4-(hydroxymethyl)piperidin-1-yl)pyrazine-2-carboxamide (may be prepared as described in Description 77; 1.3 g, 1.936 mmol) was dissolved in DCM (20 mL) and cooled in an ice bath and Dess-Martin periodinane (1.2 g, 2.83 mmol) was added, then the mixture stirred at room temperature for 2 h, then evaporated in vacuo to give a colourless gum/solid. The crude was suspended in DCM, filtered and the filtrate purified by chromatography on a 12 g silica column eluting with 0-100% EtOAc/cyclohexane. Product-containing fractions were evaporated in vacuo to give the title compound (0.72 g, 1.54 mmol, 79% yield). LCMS (high pH A): Rt=1.15 min, MH⁺=468, 470.

### Description 100

### N-((1r,4r)-4-(4-Cyano-3-(trifluoromethyl)phenoxy)cyclohexyl)-2-(4-formylpiperidin-1-yl)pyrimidine-5-carboxamide (D100)

N-((1r,4r)-4-(4-Cyano-3-(trifluoromethyl)phenoxy)cyclohexyl)-2-(4-(hydroxymethyl)piperidin-1-yl)pyrimidine-5-carboxamide (may be prepared as described in Description 78; 922 mg, 1.831 mmol) was dissolved in DCM (12 mL) and Dess-Martin periodinane (1100 mg, 2.59 mmol) was added, then the mixture stirred at room temperature for 4 h, then evaporated in vacuo. The crude was suspended in DCM (10 mL), filtered, and the filtrate purified by chromatography on a 40 g silica column eluting with 0-100% EtOAc/cyclohexane. Product-containing fractions were evaporated in vacuo. The residue was suspended in 10% MeOH in DCM (20 mL) and washed with 0.5 M NaOH (aq) (2×10 mL) (4 mL of brine was added to aid separation). The organic layer was passed through a hydrophobic frit, evaporated under a stream of nitrogen and dried in a vacuum to give the title compound (142 mg, 0.283 mmol). LCMS (formic A): Rt=1.19 min, MH⁺=502.

### Description 101

### N-((1r,4r)-4-(4-Cyano-3-(trifluoromethyl)phenoxy)cyclohexyl)-6-(4-formylpiperidin-1-yl)nicotinamide (D101)

N-((1r,4r)-4-(4-Cyano-3-(trifluoromethyl)phenoxy)cyclohexyl)-6-(4-(hydroxymethyl)piperidin-1-yl)nicotinamide (may be prepared as described in Description 80; 750 mg, 1.492 mmol) was dissolved in DCM (20 mL) and cooled in an ice bath and Dess-Martin periodinane (760 mg, 1.791 mmol) was added, then the mixture stirred at room temperature for 2 h, then evaporated in vacuo to give a colourless gum/solid. The crude was suspended in DCM, filtered, and the filtrate purified by chromatography on a 24 g silica column eluting with 0-100% EtOAc/cyclohexane. Product-containing fractions were evaporated in vacuo to give the title compound as a beige solid (680 mg, 1.359 mmol, 91% yield). LCMS (high pH A): Rt=1.15 min, MH⁺=501.

### Description 102

### N-((1r,4r)-4-(3-Chloro-4-cyanophenoxy)cyclohexyl)-6-(3-formylpiperidin-1-yl)pyridazine-3-carboxamide (D102)

Dess-martin periodinane (180 mg, 0.426 mmol) was added to a solution of N-((1r,4r)-4-(3-chloro-4-cyanophenoxy)cyclohexyl)-6-(3-(hydroxymethyl)piperidin-1-yl)pyridazine-3-carboxamide (may be prepared as described in Description 91; 200 mg, 0.426 mmol) in DCM (20 mL) at room temperature, and the mixture was stirred for 2 h, then quenched with saturated sodium bicarbonate solution, and the organic layer separated, dried and evaporated in vacuo to give a white gummy solid. This was triturated with DCM (10 mL) and filtered; the filtrate was evaporated in vacuo to give the title compound as a pale yellow gum (183 mg, 0.391 mmol, 92% yield). LCMS (high pH A): Rt=1.18 min, MH⁺=468, 470.

### Description 103

### N-((1r,4r)-4-(3-Chloro-4-cyanophenoxy)cyclohexyl)-2-(4-formylpiperidin-1-yl)pyrimidine-5-carboxamide (D103)

N-((1r,4r)-4-(3-Chloro-4-cyanophenoxy)cyclohexyl)-2-(4-(hydroxymethyl)piperidin-1-yl)pyrimidine-5-carboxamide (may be prepared as described in Description 81; 875 mg, 1.862 mmol) was dissolved in DCM (12 mL) and Dess-Martin periodinane (1100 mg, 2.59 mmol) was added, then the mixture stirred at room temperature for 4 h, then evaporated in vacuo. The crude was suspended in DCM (10 mL), filtered, and the filtrate purified by chromatography on a 40 g silica column eluting with 0-100% EtOAc/cyclohexane. Product-containing fractions were evaporated in vacuo. The residue was suspended in 10% MeOH in DCM (20 mL) and washed with 0.5 M NaOH (aq) (2×10 mL) (4 mL of brine was added to aid separation). The organic layer was passed through a hydrophobic frit, evaporated under a stream of nitrogen and dried in a vacuum oven to give the title compound (80 mg, 1.16 mmol, 9% yield). LCMS (formic A): Rt=1.09 min, MH⁺=468, 470.

### Description 104

### N-((1r,3r)-3-(3-Chloro-4-cyanophenoxy)-2,2,4,4-tetramethylcyclobutyl)-6-(4-formylpiperidin-1-yl)pyridazine-3-carboxamide (D104)

N-((1r,3r)-3-(3-chloro-4-cyanophenoxy)-2,2,4,4-tetramethylcyclobutyl)-6-(4-(hydroxymethyl)piperidin-1-yl)pyridazine-3-carboxamide (may be prepared as described in Description 72; 90 mg, 0.181 mmol) was dissolved in DCM (20 mL) and cooled in an ice bath and Dess-Martin periodinane (115 mg, 0.271 mmol) was added, then the mixture stirred at room temperature for 2 h, then evaporated in vacuo to give a colourless gum/solid. The crude was suspended in DCM, filtered and the filtrate purified by chromatography on a 12 g silica column eluting with 0-100% EtOAc/cyclohexane. Product-containing fractions were evaporated in vacuo to give the title compound (55 mg, 0.111 mmol, 61% yield). LCMS (high pH A): Rt=1.09 min, MH⁺=496, 498.

### Description 105

### N-((1r,4r)-4-(3-Chloro-4-cyanophenoxy)cyclohexyl)-4-formylbenzamide (D105)

Dess-Martin periodinane (2.64 g, 6.24 mmol) was added to N-((1r,4r)-4-(3-chloro-4-cyanophenoxy)cyclohexyl)-4-(hydroxymethyl)benzamide (may be prepared as described in Description 82; 2.0 g, 5.20 mmol) in DCM (40 mL) and stirred at room temperature for 2 h. The mixture was quenched with aq sodium thiosulphate solution (5% aq, 40 mL) and stirred for 30 min, then diluted with sodium bicarbonate solution and stirred for 20 min. The organic layer was separated, dried and evaporated in vacuo to give the title compound as a colourless solid (1.85 g, 4.83 mmol, 93% yield). LCMS (high pH A): Rt=1.16 min, MH⁺=383.

### Description 106

### N-((1r,4r)-4-(3-chloro-4-cyanophenoxy)cyclohexyl)-4-(3-oxopropyl)benzamide (D106)

Dess-Martin periodinane (61.6 mg, 0.145 mmol) was added to N-((1r,4r)-4-(3-chloro-4-cyanophenoxy)cyclohexyl)-4-(3-hydroxypropyl)benzamide (may be prepared as described in Description 83; 50 mg, 0.121 mmol) in DCM (2 mL) and stirred at room temperature for 2 h. Another portion of Dess-Martin periodinane (61.6 mg, 0.145 mmol) was added and stirring continued at RT for a further 3 h. The reaction mixture was quenched with saturated aqueous sodium bicarbonate solution (10 mL). The organic layer was separated and the aqueous layer was extracted with DCM (3×10 mL), then dried and evaporated. The residue was chromatographed [0-10% EtOAc/ethanol] to give the title compound as an off white solid (47 mg, 0.114 mmol, 94% yield). LCMS (high pH A): Rt=1.16 min, MH⁺=411, 413.

### Description 107

### N-((1r,4r)-4-(3-Chloro-4-cyanophenoxy)cyclohexyl)-4-(5-oxopentyl)benzamide (D107)

Dess-Martin periodinane (57.7 mg, 0.136 mmol) was added to N-((1r,4r)-4-(3-chloro-4-cyanophenoxy)cyclohexyl)-4-(5-hydroxypentyl)benzamide (may be prepared as described in Description 84; 50 mg, 0.113 mmol) in DCM (2 mL) and stirred at room temperature for 5 h. The reaction mixture was quenched with saturated aqueous sodium bicarbonate solution (10 mL). The organic layer was separated and the aqueous layer was extracted with DCM (3×10 mL). The combined organic extracts were dried and evaporated. The residue was chromatographed [0-50% EtOAc/cyclohexane] to give the title compound as an off white solid (17 mg, 0.039 mmol, 34% yield). LCMS (high pH A): Rt=1.25 min, MH⁺=439.

### Description 108

### N-((1r,4r)-4-(3-Chloro-4-cyanophenoxy)cyclohexyl)-4-(6-oxohexyl)benzamide (D108)

Dess-Martin periodinane (134 mg, 0.316 mmol) was added to N-((1r,4r)-4-(3-chloro-4-cyanophenoxy)cyclohexyl)-4-(6-hydroxyhexyl)benzamide (may be prepared as described in Description 85; 120 mg, 0.264 mmol) in DCM (4 mL) and stirred at room temperature for 2 h. Another portion of Dess-Martin periodinane (134 mg, 0.316 mmol) was added and stirring was continued for 30 min. The reaction mixture was quenched with saturated aqueous sodium bicarbonate solution (10 mL). The organic layer was separated and the aqueous layer was extracted with DCM (3×10 mL). The combined organic extracts were dried and evaporated to give the crude title compound as an off white solid (126 mg, 0.278 mmol) which was used in further experiments without purification. LCMS (high pH A): Rt=1.28 min, MH⁺=452.

### Description 109

### N-((1r,4r)-4-(3-Chloro-4-cyanophenoxy)cyclohexyl)-6-(4-oxopiperidin-1-yl)pyridazine-3-carboxamide (D109)

A mixture of N-((1r,4r)-4-(3-chloro-4-cyanophenoxy)cyclohexyl)-6-(1,4-dioxa-8-azaspiro[4.5]decan-8-yl)pyridazine-3-carboxamide (may be prepared as described in Description 89; 700 mg, 1.406 mmol) and aq 2 M HCl (10 mL, 20.00 mmol) were allowed to stir in a stoppered vessel at 60° C. for 3 h. Mixture was then allowed to cool to room temperature before being neutralised with 2 M aq NaOH solution. The mixture was then extracted with EtOAc (75 mL) and the organic layer passed through a hydrophobic frit. The filtrate was then evaporated to dryness to give the title compound as a yellow powder (480 mg, 1.057 mmol). LCMS (formic A): Rt=1.07 min, MH⁺=454, 456.

### Description 110

### (1-(6-(((1r,3r)-3-(3-Chloro-4-cyanophenoxy)-2,2,4,4-tetramethylcyclobutyl)carbamoyl)pyridazin-3-yl)piperidin-4-yl)methyl methanesulfonate (D110)

Ms-Cl (0.052 mL, 0.663 mmol) was added to a solution of N-((1r,3r)-3-(3-chloro-4-cyanophenoxy)-2,2,4,4-tetramethylcyclobutyl)-6-(4-(hydroxymethyl)piperidin-1-yl)pyridazine-3-carboxamide (may be prepared as described in Description 72; 220 mg, 0.442 mmol) and DIPEA (0.154 mL, 0.883 mmol) in DCM (20 mL) at 0° C. and the mixture was stirred for 2 h, allowing it to warm to room temperature, then washed with water (20 mL) and the organic layer dried and evaporated in vacuo. The residue was dissolved in DCM and loaded onto a 24 g silica column, then eluted with 0-100% EtOAc/cyclohexane and product-containing fractions evaporated in vacuo to give the title compound (125 mg, 0.217 mmol, 49% yield) as a colourless solid. LCMS (high pH A): Rt=1.35 min, MH⁺=576, 578.

### Description 111

### 6-(4-(((1r,3r)-3-(4-Cyano-3-(trifluoromethyl)phenoxy)-2,2,4,4-tetramethylcyclobutyl)carbamoyl)phenyl)hexyl 4-methylbenzenesulfonate (D111)

A mixture of N-((1r,3r)-3-(4-cyano-3-(trifluoromethyl)phenoxy)-2,2,4,4-tetramethylcyclobutyl)-4-(6-hydroxyhexyl)benzamide (may be prepared as described in Description 88; 390 mg, 0.755 mmol), tosyl-Cl (216 mg, 1.132 mmol), DMAP (9.22 mg, 0.075 mmol) and triethylamine (0.316 mL, 2.265 mmol) in DCM (10 mL) was stirred at ambient temperature for 20 h. The mixture was treated with 5% aqueous ammonium chloride (10 mL) and separated. The aqueous phase was extracted with further DCM (10 mL) and the combined organics were evaporated to dryness. The product was purified by chromatography on silica using a gradient elution from 0% to 100% methyl tert-butyl ether in cyclohexane to give the title compound (354 mg, 0.528 mmol, 70% yield). LCMS (formic A): Rt=1.59 min, MH⁺=671.

### Description 112

### 6-(4-(2-Bromoethyl)piperidin-1-yl)-N-((1r,4r)-4-(3-chloro-4-cyanophenoxy)cyclohexyl)pyridazine-3-carboxamide (D112)

A solution of N-((1r,4r)-4-(3-chloro-4-cyanophenoxy)cyclohexyl)-6-(4-(2-hydroxyethyl)piperidin-1-yl)pyridazine-3-carboxamide (may be prepared as described in Description 86; 200 mg, 0.413 mmol) in anhydrous DCM (4 mL) was added to Tetrabromomethane (206 mg, 0.620 mmol) and triphenylphosphine (163 mg, 0.620 mmol), and the mixture was stirred in a stoppered vessel at room temperature for 2 h. The solution was purified on a 12 g silica cartridge using a gradient of 0-100% EtOAc:ethanol (3:1) in TBME over 18 column volumes. The appropriate fractions were combined and the solvent evaporated in vacuo. The solid was dried under high vacuum for 16 h to give the title compound as a light brown solid (113 mg, 0.207 mmol). LCMS (formic A): Rt=1.39 min, MH⁺=546, 548, 550.

### Description 113

### N-((1r,4r)-4-(3-Chloro-4-cyanophenoxy)cyclohexyl)-5-hydroxypicolinamide (D113)

A mixture of 4-(((1r,4r)-4-aminocyclohexyl)oxy)-2-chlorobenzonitrile (may be prepared as described in Description 46; 1 g, 3.99 mmol), 5-hydroxypicolinic acid (0.694 g, 4.99 mmol) and triethylamine (2.78 mL, 19.94 mmol) in DMF (10 mL) was treated with HATU (2.123 g, 5.58 mmol) and stirred at ambient temperature for 2 h. The mixture was partitioned between EtOAc (120 mL) and brine (50 mL). The organic phase was washed with further brine (3×50 mL), dried (MgSO₄), filtered and evaporated to dryness. The product was purified by chromatography on C18 silica using a gradient elution from 20% to 80% MeCN in water (0.1% formic acid) to give the title compound (1.12 g, 3.01 mmol, 76% yield). LCMS (formic A): Rt=1.04 min, MH⁺=372, 374.

### Description 114

### 5-(3-Bromopropoxy)-N-((1r,4r)-4-(3-chloro-4-cyanophenoxy)cyclohexyl)picolinamide (D114)

A mixture of N-((1r,4r)-4-(3-chloro-4-cyanophenoxy)cyclohexyl)-5-hydroxypicolinamide (may be prepared as described in Description 113; 150 mg, 0.403 mmol), 1,3-dibromopropane (0.123 mL, 1.210 mmol) and potassium carbonate (112 mg, 0.807 mmol) in MeCN (10 mL) was stirred at 80° C. for 2 h. The product was subjected directly to purification by MDAP (formic) to give the title compound (105 mg, 0.213 mmol, 53% yield) as a white solid. LCMS (formic A): Rt=1.32 min, MH⁺=492, 494, 496.

### Description 115

### 5-(4-Bromobutoxy)-N-((1r,4r)-4-(3-chloro-4-cyanophenoxy)cyclohexyl)picolinamide (D115)

A mixture of N-((1r,4r)-4-(3-chloro-4-cyanophenoxy)cyclohexyl)-5-hydroxypicolinamide (may be prepared as described in Description 113; 150 mg, 0.403 mmol) and potassium carbonate (112 mg, 0.807 mmol) in MeCN (10 mL) was treated with 1,3-dibromopropane (0.145 mL, 1.210 mmol), and the mixture was heated at 70° C. for 18 h. The mixture was cooled, evaporated to dryness and partitioned between DCM (40 mL) and water (20 mL). The organic phase was evaporated to dryness and the product was purified by MDAP (formic) to give the title compound (117 mg, 0.231 mmol, 57% yield). LCMS (high pH A): Rt=1.37 min, MH⁺=506, 508, 510.

### Description 116

### 1-(4-(((1r,4r)-4-(3-Chloro-4-cyanophenoxy)cyclohexyl)carbamoyl)phenyl)piperidin-3-yl 4-methylbenzenesulfonate (D116)

A mixture of N-((1r,4r)-4-(3-chloro-4-cyanophenoxy)cyclohexyl)-4-(3-hydroxypiperidin-1-yl)benzamide (may be prepared as described in Description 87; 73.1 mg, 0.161 mmol) and sodium hydride, 60% by wt. (26 mg, 0.650 mmol) was stirred in THF (5 mL) at 60° C. for 15 min. 4-Methylbenzenesulfonyl chloride (125 mg, 0.656 mmol) was then added, and the mixture was stirred at 60° C. for 4 h. Sodium hydride, 60% by weight, (37.1 mg, 0.928 mmol) was further added and the reaction mixture was stirred at 60° C. for 5 min. 4-Methylbenzenesulfonyl chloride (168 mg, 0.881 mmol) was then added and the mixture was stirred at 60° C. for 1 h. Sodium hydride, 60% by weight, (37.1 mg, 0.928 mmol) was added again, and the reaction mixture was stirred at 60° C. for 5 min. 4-Methylbenzenesulfonyl chloride (165 mg, 0.866 mmol) was then added and the mixture was stirred at 60° C. for 1.5 h. The reaction mixture was cooled down for 16 h. The reaction mixture was partitioned between EtOAc (15 mL) and water (15 mL). The aqueous phase was separated. The organic phase was washed with water (10 mL) and brine (10 mL). The organic phase was dried and evaporated. The residue was chromatographed [0-50% cyclohexane/EtOAc] to give the title compound as a clear gum (22 mg, 0.036 mmol, 22% yield). LCMS (formic A): Rt=1.38 min, MH⁺=608, 610.

### Description 117

### 1-(5-(((1r,4r)-4-(3-Chloro-4-cyanophenoxy)cyclohexyl)carbamoyl)pyrimidin-2-yl)piperidine-4-carboxylic acid (D117)

N-((1r,4r)-4-(3-Chloro-4-cyanophenoxy)cyclohexyl)-2-(4-(hydroxymethyl)piperidin-1-yl)pyrimidine-5-carboxamide (may be prepared as described in Description 81; 875 mg, 1.862 mmol) was dissolved in DCM (12 mL) and Dess-Martin periodinane (1100 mg, 2.59 mmol) was added, then the mixture stirred at room temperature for 4 h, then evaporated in vacuo. The crude was suspended in DCM (10 mL), filtered, and the filtrate purified by chromatography on a 40 g silica column eluting with 0-100% EtOAc/cyclohexane. The appropriate fractions were combined and the solvent evaporated in vacuo. The residue was suspended in 10% MeOH in DCM (20 mL) and washed with 0.5 M NaOH (aq) (2×10 mL) (4 mL of brine was added to aid separation). The basic wash was acidified with 2 M HCl (aq) to pH=2 and extracted with DCM (2×10 mL). The organic extracts were combined and passed through a hydrophobic frit to give the title compound as an off white solid (37 mg, 0.076 mmol 4% yield). LCMS (formic A): Rt=1.09 min, MH⁺=484, 486.

### Description 118

### 1-(5-(((1r,4r)-4-(4-Cyano-3-(trifluoromethyl)phenoxy)cyclohexyl)carbamoyl)pyrimidin-2-yl)piperidine-4-carboxylic acid (D118)

N-((1r,4r)-4-(4-cyano-3-(trifluoromethyl)phenoxy)cyclohexyl)-2-(4-(hydroxymethyl)piperidin-1-yl)pyrimidine-5-carboxamide (may be prepared as described in Description 78; 922 mg, 1.831 mmol) was dissolved in DCM (12 mL) and Dess-Martin periodinane (1100 mg, 2.59 mmol) was added, then the mixture stirred at room temperature for 4 h, then evaporated in vacuo. The crude was suspended in DCM (10 mL), filtered, and the filtrate purified by chromatography on a 40 g silica column eluting with 0-100% EtOAc/cyclohexane. The appropriate fractions were combined and the solvent evaporated in vacuo. The residue was suspended in 10% MeOH in DCM (20 mL) and washed with 0.5 M NaOH (aq) (2×10 mL) (4 mL of brine was added to aid separation). The basic wash was acidified with 2 M HCl (aq) to pH=2 and extracted with DCM (2×10 mL). The organic extracts were combined and passed through a hydrophobic frit to give the title compound as an off white solid (37 mg, 0.076 mmol 4% yield). LCMS (formic A): Rt=1.13 min, MH⁺=518.

### Description 119

### 1-(5-(((1r,3r)-3-(3-Chloro-4-cyanophenoxy)-2,2,4,4-tetramethylcyclobutyl)carbamoyl)pyrimidin-2-yl)piperidine-4-carboxylic acid (D119)

N-((1r,3r)-3-(3-Chloro-4-cyanophenoxy)-2,2,4,4-tetramethylcyclobutyl)-2-(4-(hydroxymethyl)piperidin-1-yl)pyrimidine-5-carboxamide (may be prepared as described in Description 74; 648 mg, 1.301 mmol) was dissolved in DCM (8 mL) and Dess-Martin periodinane (1.2 g, 2.83 mmol) was added, then the mixture stirred at room temperature for 4 h, suspended in DCM (10 mL) and filtered through Celite. The filtrate was purified on a 40 g silica cartridge using a gradient of 0-100% EtOAc in cyclohexane over 12 column volumes. The appropriate fractions were combined and the solvent evaporated in vacuo. The residue was suspended in 10% MeOH in DCM (20 mL) and washed with 0.5 M NaOH (aq) (2×10 mL) (4 mL of brine was added to aid separation). The basic wash was acidified with 2 M HCl (aq) to pH=2 and extracted with DCM (2×10 mL). The organic extracts were combined and passed through a hydrophobic frit to give the title compound as an off white solid (25 mg, 0.049 mmol 3% yield). LCMS (formic A): Rt=1.09 min, MH⁺=512, 514.

### Description 120

### tert-Butyl 4-(6-(((1r,4r)-4-(3-chloro-4-cyanophenoxy)cyclohexyl)carbamoyl)pyridazin-3-yl)piperazine-1-carboxylate (D120)

A mixture of 6-chloro-N-((1r,4r)-4-(3-chloro-4-cyanophenoxy)cyclohexyl)pyridazine-3-carboxamide (may be prepared as described in Description 90; 750 mg, 1.917 mmol), tert-butyl piperazine-1-carboxylate (714 mg, 3.83 mmol), and DIPEA (743 mg, 1.004 mL, 5.75 mmol) in DMSO (10 mL) was stirred at 60° C. for 3 h. The reaction mixture was allowed to stand at room temperature overnight. The reaction mixture was diluted with water (25 mL) and stirred for 30 min. The precipitate was filtered off, washed with water and dried. The solid was suspended in diethyl ether (25 mL). The suspension was stirred for 10 min. The solid was filtered off and dried to give the title compound as a colourless solid (600 mg, 1.109 mmol, 58% yield). LCMS (high pH A): Rt=1.29 min, MH⁺=541.

### Description 121

### N-((1r,4r)-4-(3-Chloro-4-cyanophenoxy)cyclohexyl)-6-(piperazin-1-yl)pyridazine-3-carboxamide 2,2,2-trifluoroacetate (D121)

TFA (1 mL) was added to a stirred solution of tert-butyl 4-(6-(((1r,4r)-4-(3-chloro-4-cyanophenoxy)cyclohexyl)carbamoyl)pyridazin-3-yl)piperazine-1-carboxylate (may be prepared as described in Description 120; 580 mg, 1.072 mmol) in DCM (4 mL). The reaction mixture was stirred at room temperature for 1 h. The reaction mixture was diluted with diethyl ether (25 mL) and stirred for 30 min. The supernatant liquid was decanted off. The residue was triturated with diethyl ether to give the title compound as a colourless solid (521 mg, 0.939 mmol, 88% yield). LCMS (high pH A): Rt=1.01 min, MH⁺=441.

### Description 122

### tert-Butyl (2-(2-(4-(6-(((1r,4r)-4-(3-chloro-4-cyanophenoxy)cyclohexyl)carbamoyl)pyridazin-3-yl)piperazin-1-yl)ethoxy)ethyl)carbamate (D122)

A mixture of N-((1r,4r)-4-(3-chloro-4-cyanophenoxy)cyclohexyl)-6-(piperazin-1-yl)pyridazine-3-carboxamide trifluoroacetic acid salt (may be prepared as described in Description 121; 60 mg, 0.108 mmol), tert-butyl (2-(2-bromoethoxy)ethyl)carbamate (31.9 mg, 0.119 mmol) and sodium bicarbonate (45.4 mg, 0.541 mmol) in DMF (3 mL) was heated at 60° C. for 2 h. The cooled mixture was treated with DCM (20 mL) and washed with water (10 mL). The organic phase was evaporated to dryness and the product was subjected directly to purification by MDAP (formic) to give the title compound (45 mg, 0.072 mmol, 66% yield). LCMS (formic A): Rt=0.85 min, MH⁺=628.

### Description 123

### 6-(4-(2-(2-Aminoethoxy)ethyl)piperazin-1-yl)-N-((1r,4r)-4-(3-chloro-4-cyanophenoxy)cyclohexyl)pyridazine-3-carboxamide 2,2,2-trifluoroacetate (D123)

A solution of tert-butyl (2-(2-(4-(6-(((1r,4r)-4-(3-chloro-4-cyanophenoxy)cyclohexyl)carbamoyl)pyridazin-3-yl)piperazin-1-yl)ethoxy)ethyl)carbamate (may be prepared as described in Description 122; 54 mg, 0.086 mmol) in DCM (5 mL) was treated with TFA (5 mL) and stirred at ambient temperature for 1 h. The mixture was evaporated to dryness to give the title compound (36 mg, 0.056 mmol, 65% yield) which was used without further purification.

LCMS (high pH A): Rt=1.02 min, MH⁺=528, 530.

### Description 124

### 1-(4-Nitrophenyl)piperidin-4-ol (D124)

A stirring solution of 1-fluoro-4-nitrobenzene (1.4 g, 1.053 mL, 9.92 mmol), triethylamine (1.004 g, 1.383 mL, 9.92 mmol) and piperidin-4-ol (1.004 g, 9.92 mmol) in DMSO (20 mL) was heated to 80° C. for 3 h followed by cooling to room temperature. The yellow coloured solution was diluted with EtOAc (100 mL) and water (100 mL). The layers were separated and the organic layer was washed with water (100 mL), filtered through a hydrophobic frit, and concentrated in vacuo to give the title compound as a yellow coloured solid (2.08 g, 9.36 mmol, 94% yield). LCMS (high pH A): Rt=0.81 min, MH⁺=223.

### Description 125 (1-(4-Nitrophenyl)piperidin-4-yl)methanol (D125)

A stirring solution of 1-fluoro-4-nitrobenzene (1.4 g, 1.053 mL, 9.92 mmol) and piperidin-4-ylmethanol (2.5 g, 21.71 mmol) in DMSO (20 mL) was heated to 80° C. for 45 min, followed by cooling to room temperature. The yellow coloured solution was poured into 200 ml water, upon which a precipitate formed. The mixture was filtered, washing with water (30 mL), and the solid (washed into a new flask using MeOH) was concentrated in vacuo to give the title compound as a bright yellow coloured solid (2.14 g, 9.06 mmol, 91% yield). LCMS (high pH A): Rt=0.90 min, MH⁺=237.

### Description 126

### 2-(1-(4-Nitrophenyl)piperidin-4-yl)ethan-1-ol (D126)

A stirring solution of 1-fluoro-4-nitrobenzene (1.4 g, 1.053 mL, 9.92 mmol), triethylamine (1.004 g, 1.383 mL, 9.92 mmol) and 2-(piperidin-4-yl)ethan-1-ol (1.282 g, 9.92 mmol) in DMSO (20 mL) was heated to 80° C. for 16 h. The orange coloured solution was cooled to RT and diluted with EtOAc (100 mL) and water (100 mL). The layers were separated and the organic layer was washed with water (100 mL), filtered through a hydrophobic frit, and concentrated in vacuo to give the title compound as a yellow coloured solid (2.60 g, 10.39 mmol, 105% yield). LCMS (high pH A): Rt=0.97 min, MH⁺=251.

### Description 127

### 1-(4-Aminophenyl)piperidin-4-ol. (D127)

A solution of 1-(4-nitrophenyl)piperidin-4-ol (may be prepared as described in Description 124; 2.08 g, 9.36 mmol) in ethanol (100 mL) was added to a flask containing palladium on carbon (10 wt %) (0.996 g, 0.936 mmol) under nitrogen, and the flask was filled with hydrogen gas and stirred under hydrogen for 22 h. The black coloured mixture was filtered through Celite, washing the filter with ethanol (200 mL), and the filtrate was concentrated in vacuo to give the title compound as a very pale pink coloured solid (1.73 g, 9.00 mmol, 96% yield). LCMS (high pH A): Rt=0.48 min, MH⁺=193.

### Description 128

### (1-(4-Aminophenyl)piperidin-4-yl)methanol (D128)

A stirring mixture of (1-(4-nitrophenyl)piperidin-4-yl)methanol (may be prepared as described in Description 125; 2.14 g, 9.06 mmol), ammonium chloride (1.453 g, 27.2 mmol) and iron (1.517 g, 27.2 mmol) in water (15.10 mL) and ethanol (75 mL) were heated under reflux for 24 h. The mixture was filtered through celite, washing with ethanol (100 mL). The ethanol was removed in vacuo, followed by dilution of the residue with EtOAc (200 mL) and water (200 mL), and the layers were separated, and the organic layer was discarded. The aqueous layer was diluted with saturated aq. sodium hydrogen carbonate (100 mL), then extracted with EtOAc (2×200 mL), then DCM (2×200 mL). These combined organic fractions were dried, filtered, and concentrated in vacuo to give the title compound as a black coloured solid (981 mg, 4.76 mmol, 53% yield). LCMS (high pH A): Rt=0.56 min, MH⁺=207.

### Description 129

### 2-(1-(4-Aminophenyl)piperidin-4-yl)ethan-1-ol (D129)

A solution of 2-(1-(4-nitrophenyl)piperidin-4-yl)ethan-1-ol (may be prepared as described in Description 126; 2.6 g, 10.39 mmol) in ethanol (100 mL) was added to a flask containing palladium on carbon (10 weight %) (1.105 g, 1.039 mmol) under nitrogen, and the flask was filled with hydrogen gas and stirred for 24 h. The black coloured mixture was filtered through Celite, washing the filter with ethanol (200 mL), and the filtrate was concentrated in vacuo to give the title compound as a pale pink coloured solid (2.01 g, 9.12 mmol, 88% yield). LCMS (high pH A): Rt=0.62 min, MH⁺=221.

### Description 130

### Methyl 2-((4-(4-hydroxypiperidin-1-yl)phenyl)amino)-2-methylpropanoate (D130)

To 1-(4-aminophenyl)piperidin-4-ol (may be prepared as described in Description 127; 1.37 g, 7.13 mmol) and methyl 2-bromo-2-methylpropanoate (2.305 mL, 17.81 mmol) was added DIPEA (3.68 g, 4.98 mL, 28.5 mmol) and the mixture was stirred at 120° C. for 1 h followed by cooling to room temperature. The mixture was dissolved in MeOH (5 mL), EtOAc (120 mL) and water (120 mL), and the layers were separated. The aqueous layer was extracted with EtOAc (120 mL), and the combined organic layers were dried, filtered through a hydrophobic frit, and concentrated in vacuo. The residue was purified using an 80 g silica column, eluting with 0-100% EtOAc:cyclohexane, and the desired fractions were combined and concentrated in vacuo to give the title compound as a brown coloured oil (1.83 g, 6.26 mmol, 88% yield). LCMS (high pH A): Rt=0.77 min, MH⁺=293.

### Description 131

### Methyl 2-((4-(4-(hydroxymethyl)piperidin-1-yl)phenyl)amino)-2-methylpropanoate. (D131)

To (1-(4-aminophenyl)piperidin-4-yl)methanol (may be prepared as described in Description 128; 747 mg, 3.62 mmol) and methyl 2-bromo-2-methylpropanoate (1.639 g, 1171 μl, 9.05 mmol) was added DIPEA (1.872 g, 2530 μL, 14.48 mmol) and the mixture was stirred at 120° C. for 1 h followed by cooling to room temperature. The mixture was dissolved in MeOH (5 mL), EtOAc (75 mL) and water (75 mL), and the layers were separated. The aqueous layer was extracted with EtOAc (75 mL), and the combined organic layers were filtered through a hydrophobic frit and concentrated in vacuo. The residue was purified using a 40 g silica column, eluting with 0-100% EtOAc:cyclohexane, and the desired fractions were combined and concentrated in vacuo to give the title compound as a brown coloured oil (683 mg, 2.229 mmol, 62% yield). LCMS (high pH A): Rt=0.82 min, MH⁺=307.

### Description 132

### Methyl 2-((4-(4-(2-hydroxyethyl)piperidin-1-yl)phenyl)amino)-2-methylpropanoate (D132)

To 2-(1-(4-aminophenyl)piperidin-4-yl)ethan-1-ol (may be prepared as described in Description 129; 1.77 g, 8.03 mmol) and methyl 2-bromo-2-methylpropanoate (3.64 g, 2.60 mL, 20.08 mmol) was added DIPEA (4.15 g, 5.61 mL, 32.1 mmol) and the mixture was stirred at 120° C. for 1 h followed by cooling to room temperature. The mixture was dissolved in MeOH (10 mL), EtOAc (120 mL) and water (120 mL), and the layers were separated. The aqueous layer was extracted with EtOAc (120 mL), and the combined organic layers were filtered through a hydrophobic frit and concentrated in vacuo. The residue was purified using an 80 g silica column, eluting with 0-100% EtOAc:cyclohexane, and the desired fractions were combined and concentrated in vacuo to give the title compound as a brown coloured oil that solidified upon standing (2.58 g, 8.05 mmol, 100% yield). LCMS (high pH A): Rt=0.89 min, MH⁺=321.

### Description 133

### 4-(3-(4-(4-Hydroxypiperidin-1-yl)phenyl)-4,4-dimethyl-5-oxo-2-thioxoimidazolidin-1-yl)-2-(trifluoromethyl)benzonitrile (D133)

A stirring solution of methyl 2-((4-(4-hydroxypiperidin-1-yl)phenyl)amino)-2-methylpropanoate (may be prepared as described in Description 130; 0.819 g, 2.80 mmol) and 4-isothiocyanato-2-(trifluoromethyl)benzonitrile (0.639 g, 2.80 mmol) in isopropyl acetate (10.00 mL) and DMSO (5 mL) was heated to 80° C. for 2 h followed by cooling to room temperature. The golden coloured solution was diluted with EtOAc (50 mL) and water (50 mL), and the layers were separated. The organic layer was filtered through a hydrophobic frit and concentrated in vacuo. The residue was purified using a 40 g silica column, eluting with 0-100% EtOAc:cyclohexane, and the desired fractions were combined and concentrated in vacuo to give the title compound as a yellow coloured gum (1.33 g, 2.72 mmol, 97% yield). LCMS (high pH A): Rt=1.15 min, MH⁺=489.

### Description 134

### 4-(3-(4-(4-(Hydroxymethyl)piperidin-1-yl)phenyl)-4,4-dimethyl-5-oxo-2-thioxoimidazolidin-1-yl)-2-(trifluoromethyl)benzonitrile (D134)

A stirring solution of methyl 2-((4-(4-(hydroxymethyl)piperidin-1-yl)phenyl)amino)-2-methylpropanoate (may be prepared as described in Description 131; 584 mg, 1.906 mmol) and 4-isothiocyanato-2-(trifluoromethyl)benzonitrile (435 mg, 1.906 mmol) in isopropyl acetate (10.00 mL) and DMSO (5 mL) was heated to 80° C. for 2 h followed by cooling to room temperature. The golden coloured solution was diluted with EtOAc (40 mL) and water (40 mL), and the layers were separated. The organic layer was filtered through a hydrophobic frit and concentrated in vacuo. The residue was purified using a 40 g silica column, eluting with 0-100% EtOAc:cyclohexane, and the desired fractions were combined and concentrated in vacuo to give the title compound as a yellow coloured gum (906 mg, 1.803 mmol, 95% yield). LCMS (high pH A): Rt=1.20 min, MH⁺=503.

### Description 135

### 4-(3-(4-(4-(2-Hydroxyethyl)piperidin-1-yl)phenyl)-4,4-dimethyl-5-oxo-2-thioxoimidazolidin-1-yl)-2-(trifluoromethyl)benzonitrile (D135)

A stirring solution of methyl 2-((4-(4-(2-hydroxyethyl)piperidin-1-yl)phenyl)amino)-2-methylpropanoate (may be prepared as described in Description 132; 0.974 g, 3.04 mmol) and 4-isothiocyanato-2-(trifluoromethyl)benzonitrile (0.694 g, 3.04 mmol) in isopropyl acetate (10.00 mL) and DMSO (5 mL) was heated to 80° C. for 2 h followed by cooling to room temperature. The golden coloured solution was diluted with EtOAc (40 mL) and water (40 mL), and the layers were separated. The organic layer was filtered through a hydrophobic frit and concentrated in vacuo. The residue was purified using a 40 g silica column, eluting with 0-100% EtOAc:cyclohexane, and the desired fractions were combined and concentrated in vacuo to give the title compound as a yellow coloured solid foam (1.20 g, 2.323 mmol, 76% yield). LCMS (high pH A): Rt=1.26 min, MH⁺=517.

### Description 136

### 1-(4-(3-(4-Cyano-3-(trifluoromethyl)phenyl)-5,5-dimethyl-4-oxo-2-thioxoimidazolidin-1-yl)phenyl)piperidin-4-yl 4-methylbenzenesulfonate (D136)

To a stirring solution of 4-(3-(4-(4-hydroxypiperidin-1-yl)phenyl)-4,4-dimethyl-5-oxo-2-thioxoimidazolidin-1-yl)-2-(trifluoromethyl)benzonitrile (may be prepared as described in Description 133; 1.33 g, 2.72 mmol) and 4-methylbenzenesulphonyl chloride (1.557 g, 8.17 mmol) in DCM (20 mL) was added triethylamine (1.102 g, 1.518 mL, 10.89 mmol), followed by stirring for 18 h. The pale yellow coloured mixture was diluted with EtOAc (100 mL) and water (50 mL). The layers were separated, and the aqueous layer was extracted with EtOAc (50 mL). The combined organic layers were filtered through a hydrophobic frit and concentrated in vacuo. The crude product was purified using a 40 g silica column, eluting with 0-100% EtOAc:cyclohexane, and the desired fractions were combined and concentrated in vacuo to give the title compound as an orange coloured gum which solidified upon standing (750 mg, 1.167 mmol, 43% yield). LCMS (high pH A): Rt=1.43 min, MH⁺=643.

### Description 137

### (1-(4-(3-(4-Cyano-3-(trifluoromethyl)phenyl)-5,5-dimethyl-4-oxo-2-thioxoimidazolidin-1-yl)phenyl)piperidin-4-yl)methyl 4-methylbenzenesulfonate (D137)

To a stirring solution of 4-(3-(4-(4-(hydroxymethyl)piperidin-1-yl)phenyl)-4,4-dimethyl-5-oxo-2-thioxoimidazolidin-1-yl)-2-(trifluoromethyl)benzonitrile (may be prepared as described in Description 134; 906 mg, 1.803 mmol) and 4-methylbenzenesulphonyl chloride (1031 mg, 5.41 mmol) in DCM (20 mL) was added triethylamine (730 mg, 1.005 mL, 7.21 mmol), followed by stirring for 16 h. The pale brown coloured solution was diluted with EtOAc (70 mL) and water (70 mL), and brine (30 mL). The layers were separated, and the aqueous layer was extracted with EtOAc (50 mL). The combined organic layers were dried, filtered through a hydrophobic frit, and concentrated in vacuo. The crude product was purified using a 40 g silica column, eluting with 0-70% EtOAc:cyclohexane, and the desired fractions were combined and concentrated in vacuo to give the title compound as a yellow coloured gum (904 mg, 1.376 mmol, 76% yield). LCMS (high pH A): Rt=1.46 min, MH⁺=657.

### Description 138

### 2-(1-(4-(3-(4-Cyano-3-(trifluoromethyl)phenyl)-5,5-dimethyl-4-oxo-2-thioxoimidazolidin-1-yl)phenyl)piperidin-4-yl)ethyl 4-methylbenzenesulfonate (D138)

To a stirring solution of 4-(3-(4-(4-(2-hydroxyethyl)piperidin-1-yl)phenyl)-4,4-dimethyl-5-oxo-2-thioxoimidazolidin-1-yl)-2-(trifluoromethyl)benzonitrile (may be prepared as described in Description 135; 1.20 g, 2.323 mmol) and 4-methylbenzenesulfonyl chloride (1.329 g, 6.97 mmol) in DCM (20 mL) was added triethylamine (0.94 g, 1.295 mL, 9.29 mmol), followed by stirring for 18 h. The pale yellow coloured mixture was diluted with EtOAc (100 mL) and water (50 mL). The layers were separated, and the aqueous layer was extracted with EtOAc (50 mL). The combined organic layers were filtered through a hydrophobic frit and concentrated in vacuo. The crude product was purified using a 40 g silica column, eluting with 0-70% EtOAc:cyclohexane, and the desired fractions were combined and concentrated in vacuo to give the title compound as a pale yellow coloured solid foam (1.39 g, 2.072 mmol, 89% yield). LCMS (high pH A): Rt=1.49 min, MH⁺=671.

### Description 139

### 4-(3-(4-(3-Hydroxypropoxy)phenyl)-4,4-dimethyl-5-oxo-2-thioxoimidazolidin-1-yl)-2-(trifluoromethyl)benzonitrile (D139)

To a stirring suspension of 4-(3-(4-hydroxyphenyl)-4,4-dimethyl-5-oxo-2-thioxoimidazolidin-1-yl)-2-(trifluoromethyl)benzonitrile (commercially available from e.g. Ambeed; 200 mg, 0.493 mmol) and potassium carbonate (170 mg, 1.233 mmol) in DMF (5 mL) was added 3-bromopropan-1-ol (137 mg, 0.089 mL, 0.987 mmol), followed by stirring for 64 h. The mixture was diluted with EtOAc (20 mL) and water (20 mL). The layers were separated, and the aqueous layer was extracted with EtOAc (30 mL). The combined organic layers were filtered through a hydrophobic frit and concentrated in vacuo. The crude product was loaded onto a 24 g silica column and eluted with 0-100% EtOAc:cyclohexane, and the desired fractions were combined and concentrated in vacuo to give the title compound as a colourless oil (262 mg, 0.565 mmol, 115% yield). LCMS (high pH A): Rt=1.16 min, MH⁺=464.

### Description 140

### 4-(3-(4-(4-Hydroxybutoxy)phenyl)-4,4-dimethyl-5-oxo-2-thioxoimidazolidin-1-yl)-2-(trifluoromethyl)benzonitrile (D140)

To a stirring suspension of 4-(3-(4-hydroxyphenyl)-4,4-dimethyl-5-oxo-2-thioxoimidazolidin-1-yl)-2-(trifluoromethyl)benzonitrile (commercially available from e.g. Ambeed; 200 mg, 0.493 mmol) and potassium carbonate (170 mg, 1.233 mmol) in DMF (5 mL) was added 4-bromobutan-1-ol (0.104 mL, 0.987 mmol), followed by stirring for 64 h. A further portion of 4-bromobutan-1-ol (0.104 mL, 0.987 mmol) was added followed by an additional portion of potassium carbonate (170 mg, 1.233 mmol) and stirring for 24 h. The pale yellow coloured mixture was heated to 60° C. for 5 h followed by cooling to room temperature. Final portions of potassium carbonate (170 mg, 1.233 mmol) and 4-bromobutan-1-ol (0.104 mL, 0.987 mmol) were added followed by stirring for 16 h. The pale yellow coloured solution was diluted with EtOAc (20 mL) and water (20 mL). The layers were separated, and the aqueous layer was extracted with EtOAc (20 mL). The combined organic layers were filtered through a hydrophobic frit and concentrated in vacuo. The crude product was loaded onto a 24 g silica column using minimum DCM, and eluted with 0-100% EtOAc:cyclohexane and the desired fractions were combined and concentrated in vacuo to give the title compound as a colourless oil (151 mg, 0.316 mmol, 64% yield). LCMS (high pH A): Rt=1.20 min, MH⁺=478.

### Description 141

### 3-(4-(3-(4-Cyano-3-(trifluoromethyl)phenyl)-5,5-dimethyl-4-oxo-2-thioxoimidazolidin-1-yl)phenoxy)propyl 4-methylbenzenesulfonate (D141)

To a stirring solution of 4-(3-(4-(3-hydroxypropoxy)phenyl)-4,4-dimethyl-5-oxo-2-thioxoimidazolidin-1-yl)-2-(trifluoromethyl)benzonitrile (may be prepared as described in Description 139; 210 mg, 0.453 mmol) and 4-methylbenzenesulfonyl chloride (259 mg, 1.359 mmol) in DCM (5 mL) was added triethylamine (183 mg, 0.253 mL, 1.812 mmol), followed by stirring for 5 h. The pale yellow coloured mixture was diluted with EtOAc (15 mL) and water (15 mL). The layers were separated, and the aqueous layer was extracted with EtOAc (15 mL). The combined organic layers were filtered through a hydrophobic frit and concentrated in vacuo. The crude product was purified using a 12 g silica column, eluting with 0-70% EtOAc:cyclohexane, and the desired fractions were combined and concentrated in vacuo to give the title compound as a colourless gum (238 mg, 0.385 mmol, 85% yield). LCMS (high pH A): Rt=1.43 min, MH⁺=618.

### Description 142

### 4-(4-(3-(4-Cyano-3-(trifluoromethyl)phenyl)-5,5-dimethyl-4-oxo-2-thioxoimidazolidin-1-yl)phenoxy)butyl 4-methylbenzenesulfonate (D142)

To a stirring solution of 4-(3-(4-(4-hydroxybutoxy)phenyl)-4,4-dimethyl-5-oxo-2-thioxoimidazolidin-1-yl)-2-(trifluoromethyl)benzonitrile (may be prepared as described in Description 140; 151 mg, 0.316 mmol) and 4-methylbebzenesulfonyl chloride (181 mg, 0.949 mmol) in DCM (3 mL) was added triethylamine (128 mg, 0.176 mL, 1.265 mmol), followed by stirring for 5 h. The pale yellow coloured mixture was diluted with EtOAc (15 mL) and water (15 mL). The layers were separated, and the aqueous layer was extracted with EtOAc (15 mL). The combined organic layers were filtered through a hydrophobic frit and concentrated in vacuo. The crude product was purified using a 12 g silica column, eluting with 0-70% EtOAc:cyclohexane, and the desired fractions were combined and concentrated in vacuo to give the title compound as a colourless gum (154 mg, 0.244 mmol, 77% yield). LCMS (high pH A): Rt=1.46 min, MH⁺=632.

### Description 143

### tert-Butyl (2R,5S)-4-(3-chloro-4-cyanophenyl)-2,5-dimethylpiperazine-1-carboxylate (D143)

A mixture of 2-chloro-4-fluorobenzonitrile (5 g, 32.1 mmol), potassium carbonate (8.88 g, 64.3 mmol) and tert-butyl (2R,5S)-2,5-dimethylpiperazine-1-carboxylate (6 g, 28.0 mmol) in DMSO (40 mL) and Water (4 mL) and stirred at 60° C. for 46 h. The mixture was allowed to cool to room temperature, diluted with water (40 mL), then extracted with EtOAc (2×40 mL). The organic extracts were combined, washed with brine (50 mL), and passed through a hydrophobic frit. The filtrate was concentrated under vacuum and the gum loaded in CHCl₃ (10 mL) and purified on a 120 g silica cartridge using a gradient of 0-50% EtOAc in cyclohexane over 10 column volumes. The appropriate fractions were combined and the solvent evaporated in vacuo to give to give the title compound as a pale yellow solid (7.2 g, 20.58 mmol, 64% yield). The material was used in the next step without further purification. LCMS (high pH A): Rt=1.36 min, M-^{t}Bu⁺294.0, 296.0.

### Description 142

### 2-Chloro-4-((2S,5R)-2,5-dimethylpiperazin-1-yl)benzonitrile (D142)

4 M HCl in Dioxane (20 mL, 80 mmol) was added to a solution of tert-butyl (2R,5S)-4-(3-chloro-4-cyanophenyl)-2,5-dimethylpiperazine-1-carboxylate (may be prepared as described in Description 143; 7.2 g, 20.58 mmol) in DCM (40 mL) and the mixture was stirred overnight at room temperature. The resulting suspension was diluted with MTBE (50 mL) and filtered to give a hygroscopic solid. The solid was dissolved in water (50 mL) and basified with solid potassium carbonate (10 g), then extracted with EtOAc (2×50 mL) and the organics dried and evaporated in vacuo to give the title compound as a pale yellow gum (4.7 g, 18.82 mmol, 91% yield). LCMS (high pH A): Rt=0.95 min, MH⁺=250, 252.

### Description 143

### 2-Chloro-4-((2R,5S)-2,5-dimethylpiperazin-1-yl)benzonitrile (D143)

Material was prepared in a similar manner to D142 from tert-butyl (2S,5R)-2,5-dimethylpiperazine-1-carboxylate (6.89 g) to give the title compound as a yellow gum (3.5 g).

LCMS (high pH A): Rt=0.96 min, MH⁺=250.0, 252.0.

### Description 144

### 4-((5-Nitropyridin-2-yl)oxy) butan-1-ol (D144)

Sodium hydride (0.631 g, 15.77 mmol) was added to a solution of butane-1,4-diol (1.137 g, 12.62 mmol) in THF (10 mL) and the mixture was stirred for 10 min, then 2-chloro-5-nitropyridine (1 g, 6.31 mmol) was added and the mixture stirred at room temperature for 1 h. The mixture was quenched with water and extracted with EtOAc, the organics dried and evaporated in vacuo to give a pale yellow liquid. This was dissolved in DCM and loaded onto a 40 g silica column, then eluted with 0-100% EtOAc/cyclohexane and product-containing fractions evaporated in vacuo to give the title compound as a pale yellow oil (1.04 g, 4.90 mmol, 78% yield). LCMS (high pH A): Rt=0.77 min, MH⁺=213.0.

### Description 145

### 2-(4-((tert-Butyldimethylsilyl)oxy)butoxy)-5-nitropyridine (D145)

TBDMS-Cl (0.813 g, 5.39 mmol) was added to a solution of 4-((5-nitropyridin-2-yl)oxy) butan-1-ol (may be prepared as described in Description 144; 1.04 g, 4.90 mmol) and imidazole (0.434 g, 6.37 mmol) in DCM (20 mL) at room temperature and the mixture was stirred for 1 h, then washed with water and the organic layer dried and evaporated in vacuo to give the title compound as a colourless liquid (1.61 g, 4.93 mmol) which was used in the next step without purification. LCMS (high pH A): Rt=1.62 min, MH⁺=326.9.

### Description 146

### 6-(4-((tert-Butyldimethylsilyl)oxy)butoxy)pyridin-3-amine (D146)

2-(4-((tert-Butyldimethylsilyl)oxy)butoxy)-5-nitropyridine (may be prepared as described in Description 146; 1.6 g, 4.90 mmol) was dissolved in MeOH (50 mL) and added to a purged hydrogenation flask containing Pd—C 5% on carbon (0.5 g, 4.70 mmol), then stirred under a hydrogen atmosphere for 2 h at room temperature. The mixture was filtered through Celite under nitrogen and the solvent evaporated in vacuo to give the title compound as a pale yellow oil (1.51 g, 5.09 mmol) which was used in the next step without purification. LCMS (high pH A): Rt=1.18 min, MH⁺=297.0.

### Description 147

### Phenyl (6-(4-((tert-butyldimethylsilyl)oxy)butoxy)pyridin-3-yl)carbamate (D147)

6-(4-((tert-Butyldimethylsilyl)oxy)butoxy)pyridin-3-amine (may be prepared as described in description 146; 1.51 g, 5.09 mmol) was dissolved in DCM (30 mL) and cooled in an ice bath, then phenyl carbonochloridate (1.037 g, 6.62 mmol) and N-ethyl-N-isopropylpropan-2-amine (1.317 g, 10.19 mmol) were added dropwise and the mixture stirred for 1 h, then washed with water and the organics dried and evaporated in vacuo, The crude was dissolved in DCM and loaded onto a 40 g silica column and eluted with 0-50% EtOAc cyclohexane and product-containing fractions evaporated in vacuo to give the title compound as a pale yellow solid (1.48 g, 3.55 mmol, 70% yield). LCMS (high pH A): Rt=1.58 min, MH⁺=417.1.

### Description 148

### (2R,5S)—N-(6-(4-((tert-Butyldimethylsilyl)oxy)butoxy)pyridin-3-yl)-4-(3-chloro-4-cyanophenyl)-2,5-dimethylpiperazine-1-carboxamide (D148)

2-Chloro-4-((2S,5R)-2,5-dimethylpiperazin-1-yl)benzonitrile (may be prepared as described in Description 142; 210 mg, 0.840 mmol) and phenyl (6-(4-((tert-butyldimethylsilyl)oxy)butoxy)pyridin-3-yl)carbamate (may be prepared as described in Description 147; 350 mg, 0.840 mmol) were dissolved in MeCN (10 mL) and heated at 60° C. for 2 h, then the solvent was evaporated in vacuo to give a brown gum. The crude was dissolved in DCM and loaded onto a 24 g silica column, then eluted with 0-100% EtOAc/cyclohexane and product-containing fractions evaporated in vacuo to give the title compound as a colourless gum (0.35 g, 0.612 mmol, 73% yield). LCMS (high pH A): Rt=1.60 min, MH⁺=572.1, 574.1.

### Description 149

### (2R,5S)-4-(3-Chloro-4-cyanophenyl)-N-(6-(4-hydroxybutoxy)pyridin-3-yl)-2,5-dimethylpiperazine-1-carboxamide (D149)

4 M HCl in dioxane (2 mL, 8.00 mmol) was added to a solution of (2R,5S)—N-(6-(4-((tert-butyldimethylsilyl)oxy)butoxy)pyridin-3-yl)-4-(3-chloro-4-cyanophenyl)-2,5-dimethylpiperazine-1-carboxamide (may be prepared as described in Description 148; 350 mg, 0.612 mmol) in DCM (10 mL) at room temp and the mixture stirred for 30 min then evaporated in vacuo to give a pale yellow foam. This was partitioned between aq potassium carbonate (10% aq, 10 mL) and EtOAc (20 mL). The organic layer was dried and evaporated in vacuo to give the title compound as a colourless foam (275 mg, 0.600 mmol, 98% yield) which was used in the next step without purification. LCMS (high pH A): Rt=1.02 min, MH⁺=458.0. 460.0.

### Description 150

### (2R,5S)-4-(3-Chloro-4-cyanophenyl)-2,5-dimethyl-N-(6-(4-oxobutoxy)pyridin-3-yl)piperazine-1-carboxamide (D150)

(2R,5S)-4-(3-Chloro-4-cyanophenyl)-N-(6-(4-hydroxybutoxy)pyridin-3-yl)-2,5-dimethylpiperazine-1-carboxamide (may be prepared as described in Description 149; 275 mg, 0.600 mmol) was dissolved in DCM (20 mL) and cooled in an ice bath and Dess-Martin periodinane (331 mg, 0.781 mmol) was added, then the mixture stirred at room temperature for 2 h, then evaporated in vacuo to give a gummy solid. The crude was suspended in DCM and loaded onto a 24 g silica column, then eluted with 0-100% EtOAc/cyclohexane and product-containing fractions evaporated in vacuo to give the title compound as a colourless foam (210 mg, 0.461 mmol, 77% yield). LCMS (high pH A): Rt=1.09 min, MH⁺=456.0, 458.0.

### Description 151

### 4-(((tert-Butyldimethylsilyl)oxy)methyl)piperidine (D151)

TBDMS-Cl (7.85 g, 52.1 mmol) was added to a solution of piperidin-4-ylmethanol (3 g, 26.0 mmol) and imidazole (3.90 g, 57.3 mmol) in DCM (50 mL) at 0° C. and the mixture was stirred for 1 h, then washed with water and the organic layer dried and evaporated in vacuo to give the title compound as a colourless liquid (10.1 g, 44.0 mmol) which was used in the next step without purification.

### Description 152

### 2-(4-(((tert-Butyldimethylsilyl)oxy)methyl)piperidin-1-yl)-5-nitropyridine (D152)

2-Chloro-5-nitropyridine (0.7 g, 4.42 mmol) was dissolved in DCM (20 mL) and DIPEA (2.313 mL, 13.25 mmol) was added, followed by 4-(((tert-butyldimethylsilyl)oxy)methyl)piperidine (may be prepared as described in Description 151; 2.026 g, 8.83 mmol). The mixture was stirred at room temperature for 30 min, then diluted with EtOAc (30 mL) and washed with water (3×30 mL) and brine (30 mL). The organic layer was dried and evaporated in vacuo to give an orange solid. The crude was dissolved in DCM and loaded onto a 40 g silica column and eluted with 0-50% EtOAc/cyclohexane. Product-containing fractions were evaporated in vacuo to give the title compound as a pale yellow solid (1.55 g, 4.41 mmol, 100% yield). LCMS (high pH A): Rt=1.65 min, MH⁺=352.0.

### Description 153

### 6-(4-(((tert-Butyldimethylsilyl)oxy)methyl)piperidin-1-yl)pyridin-3-amine (D153)

2-(4-(((tert-Butyldimethylsilyl)oxy)methyl)piperidin-1-yl)-5-nitropyridine (may be prepared as described in Description 152; 1.55 g, 4.41 mmol) was dissolved in Ethanol (100 mL) and added to a purged hydrogenation flask containing Pd—C 10% (100 mg, 0.940 mmol), then stirred under a hydrogen atmosphere for 2 h. The mixture was filtered under nitrogen and the filtrate evaporated in vacuo to give to give the title compound as a pale yellow gum (1.45 g, 4.51 mmol, 102% yield). LCMS (high pH A): Rt=0.84 min, MH⁺=322.1.

### Description 154

### Phenyl (6-(4-(((tert-butyldimethylsilyl)oxy)methyl)piperidin-1-yl)pyridin-3-yl)carbamate (D154)

6-(4-(((tert-Butyldimethylsilyl)oxy)methyl)piperidin-1-yl)pyridin-3-amine (may be prepared as described in Description 153; 1.4 g, 4.37 mmol) was dissolved in DCM (30 mL) and cooled in an ice bath, then phenyl carbonochloridate (0.712 mL, 5.68 mmol) and DIPEA (1.526 mL, 8.73 mmol) were added dropwise and the mixture stirred for 1 h, then washed with water and the organics dried and evaporated in vacuo, The crude was dissolved in DCM and loaded onto a 40 g silica column and eluted with 0-50% EtOAc cyclohexane and product-containing fractions evaporated in vacuo to give the title compound as a pale pink solid (1.58 g, 3.58 mmol, 82% yield). LCMS (high pH A): Rt=1.27 min, MH⁺=442.1.

### Description 155

### (2R,5S)—N-(6-(4-(((tert-Butyldimethylsilyl)oxy)methyl)piperidin-1-yl)pyridin-3-yl)-4-(3-chloro-4-cyanophenyl)-2,5-dimethylpiperazine-1-carboxamide (D155)

2-Chloro-4-((2S,5R)-2,5-dimethylpiperazin-1-yl)benzonitrile (may be prepared as described in Description 142; 0.187 g, 0.747 mmol) and phenyl (6-(4-(((tert-butyldimethylsilyl)oxy)methyl)piperidin-1-yl)pyridin-3-yl)carbamate (may be prepared as described in Description 154; 0.33 g, 0.747 mmol) were dissolved in MeCN (10 mL) and heated at 60° C. for 2 h, then the solvent was evaporated in vacuo to give a brown gum. The crude was dissolved in DCM and loaded onto a 24 g silica column, then eluted with 0-100% EtOAc/cyclohexane and product-containing fractions evaporated in vacuo to give the title compound as a colourless gum (321 mg, 0.537 mmol, 72% yield). LCMS (high pH A): Rt=1.62 min, MH⁺=597.2, 599.2.

### Description 156

### (2R,5S)-4-(3-Chloro-4-cyanophenyl)-N-(6-(4-(hydroxymethyl)piperidin-1-yl)pyridin-3-yl)-2,5-dimethylpiperazine-1-carboxamide (D156)

4 M HCl in dioxane (2 mL, 8.00 mmol) was added to a solution of (2R,5S)—N-(6-(4-(((tert-butyldimethylsilyl)oxy)methyl)piperidin-1-yl)pyridin-3-yl)-4-(3-chloro-4-cyanophenyl)-2,5-dimethylpiperazine-1-carboxamide (may be prepared as described in Description 155; 320 mg, 0.536 mmol) in DCM (10 mL) at room temp and the mixture stirred for 30 min then evaporated in vacuo to give a pale yellow foam. This was partitioned between aq potassium carbonate (10% aq, 10 mL) and EtOAc (20 mL). The organic layer was dried and evaporated in vacuo to give the title compound as a colourless foam (230 mg, 0.476 mmol, 89% yield) which was used in the next step without purification.

### Description 157

### (2R,5S)-4-(3-Chloro-4-cyanophenyl)-N-(6-(4-formylpiperidin-1-yl)pyridin-3-yl)-2,5-dimethylpiperazine-1-carboxamide (D157)

(2R,5S)-4-(3-Chloro-4-cyanophenyl)-N-(6-(4-(hydroxymethyl)piperidin-1-yl)pyridin-3-yl)-2,5-dimethylpiperazine-1-carboxamide (may be prepared as described in Description 156; 230 mg, 0.476 mmol) was dissolved in DCM (20 mL) and cooled in an ice bath and Dess-Martin periodinane (263 mg, 0.619 mmol) was added, then the mixture stirred at room temperature for 2 h, then evaporated in vacuo to give a gummy solid. The crude was suspended in DCM and loaded onto a 24 g silica column, then eluted with 0-100% EtOAc/cyclohexane and product-containing fractions evaporated in vacuo to give the title compound as a colourless foam (105 mg, 0.218 mmol, 46% yield). LCMS (high pH A): Rt=1.09 min, MH⁺=597.2, 599.2.

### Description 158

### 2-(4-(((tert-Butyldimethylsilyl)oxy)methyl)piperidin-1-yl)-5-nitropyrimidine (D158)

4-(((tert-Butyldimethylsilyl)oxy)methyl)piperidine (may be prepared as described in Description 151; 3.2 g, 13.95 mmol) and DIPEA (3.65 mL, 20.92 mmol) were dissolved in DCM (50 mL) and the solution cooled in an ice bath, then 2-chloro-5-nitropyrimidine (2.002 g, 12.55 mmol) was added and the mixture stirred at 0° C. for 1 h, then washed with water, the organic layer dried and evaporated in vacuo to give a pale yellow solid. The crude was dissolved in DCM and loaded onto a 80 g silica column and eluted with 0-20% EtOAc/cyclohexane. Product-containing fractions were evaporated in vacuo to give the title compound as a colourless solid (2.48 g, 7.04 mmol, 50% yield). LCMS (high pH A): Rt=1.69 min, MH⁺=353.1

### Description 159

### 2-(4-(((tert-Butyldimethylsilyl)oxy)methyl)piperidin-1-yl)pyrimidin-5-amine (D159)

2-(4-(((tert-Butyldimethylsilyl)oxy)methyl)piperidin-1-yl)-5-nitropyrimidine (may be prepared as described in Description 158; 2.48 g, 7.04 mmol) was dissolved in Ethanol (100 mL) and added to a purged hydrogenation flask containing Pd—C 10% (100 mg, 0.940 mmol), then stirred under a hydrogen atmosphere for 2 h. The mixture was filtered under nitrogen and the filtrate evaporated in vacuo to give the title compound as a pale yellow gum (2.32 g, 7.19 mmol, 102% yield). LCMS (high pH A): Rt=1.38 min, MH⁺=323.2.

### Description 160

### Phenyl (2-(4-(((tert-butyldimethylsilyl)oxy)methyl)piperidin-1-yl)pyrimidin-5-yl)carbamate (D160)

2-(4-(((tert-Butyldimethylsilyl)oxy)methyl)piperidin-1-yl)pyrimidin-5-amine (may be prepared as described in Description 159; 1.76 g, 5.46 mmol) and DIPEA (1.906 mL, 10.91 mmol) were dissolved in DCM (20 mL), the solution cooled in an ice bath, then phenyl chloroformate (0.822 mL, 6.55 mmol) was added and the mixture was stirred for 1 h, allowing it to warm to room temperature. The solution was washed with water and the organic layer dried and evaporated in vacuo to give a brown gum. The crude product was dissolved in DCM and loaded onto a 24 g silica column and eluted with 0-30% EtOAc/cyclohexane and product-containing fractions evaporated in vacuo to give the title compound as a pale yellow solid (1.98 g, 4.47 mmol, 82% yield). LCMS (high pH A): Rt=1.64 min, MH⁺=443.1.

### Description 161

### (2R,5S)—N-(2-(4-(((tert-Butyldimethylsilyl)oxy)methyl)piperidin-1-yl)pyrimidin-5-yl)-4-(3-chloro-4-cyanophenyl)-2,5-dimethylpiperazine-1-carboxamide (D161)

2-Chloro-4-((2S,5R)-2,5-dimethylpiperazin-1-yl)benzonitrile (may be prepared as described in Description 142; 254 mg, 1.017 mmol) and phenyl (2-(4-(((tert-butyldimethylsilyl)oxy)methyl)piperidin-1-yl)pyrimidin-5-yl)carbamate (may be prepared as described in Description 160; 450 mg, 1.017 mmol) were dissolved in MeCN (10 mL) and heated at 60° C. for 2 h, then the solvent was evaporated in vacuo to give a brown gum. The crude was dissolved in DCM and loaded onto a 24 g silica column, then eluted with 0-100% EtOAc/cyclohexane and product-containing fractions evaporated in vacuo to give the title compound as a colourless gum (0.54 g, 0.903 mmol, 89% yield). LCMS (high pH A): Rt=1.62 min, MH⁺=598.2, 600.2.

### Description 162

### (2R,5S)-4-(3-Chloro-4-cyanophenyl)-N-(2-(4-(hydroxymethyl)piperidin-1-yl)pyrimidin-5-yl)-2,5-dimethylpiperazine-1-carboxamide (D162)

4 M HCl in dioxane (2 mL, 8.00 mmol) was added to a solution of (2R,5S)—N-(2-(4-(((tert-butyldimethylsilyl)oxy)methyl)piperidin-1-yl)pyrimidin-5-yl)-4-(3-chloro-4-cyanophenyl)-2,5-dimethylpiperazine-1-carboxamide (may be prepared as described in Description 161; 0.51 g, 0.852 mmol) in DCM (10 mL) at room temperature and the mixture stirred for 30 min then evaporated in vacuo to give a pale yellow foam. This was partitioned between aq potassium carbonate (10% aq, 10 mL) and EtOAc (20 mL). The organic layer was dried and evaporated in vacuo to give the title compound (430 mg, 0.888 mmol, 104% yield) as a colourless foam which was used in the next step without purification. LCMS (high pH A): Rt=1.00 min, MH⁺=484.1, 486.1.

### Description 163

### (2R,5S)-4-(3-Chloro-4-cyanophenyl)-N-(2-(4-formylpiperidin-1-yl)pyrimidin-5-yl)-2,5-dimethylpiperazine-1-carboxamide (D163)

(2R,5S)-4-(3-chloro-4-cyanophenyl)-N-(2-(4-(hydroxymethyl)piperidin-1-yl)pyrimidin-5-yl)-2,5-dimethylpiperazine-1-carboxamide (may be prepared as described in Description 162; 400 mg, 0.826 mmol) was dissolved in DCM (20 mL) and cooled in an ice bath and Dess-Martin periodinane (456 mg, 1.074 mmol) was added, then the mixture stirred at room temperature for 2 h, then evaporated in vacuo to give a gummy solid. The crude was suspended in DCM and loaded onto a 24 g silica column, then eluted with 0-100% EtOAc/cyclohexane and product-containing fractions evaporated in vacuo to give the title compound as a colourless foam (210 mg, 0.436 mmol, 53% yield). LCMS (high pH A): Rt=1.09 min, MH⁺=482.1, 484.1.

### Description 164

### (2S,5R)-4-(3-chloro-4-cyanophenyl)-N-(2-(4-formylpiperidin-1-yl)pyrimidin-5-yl)-2,5-dimethylpiperazine-1-carboxamide (D164)

(2S,5R)-4-(3-Chloro-4-cyanophenyl)-N-(2-(4-formylpiperidin-1-yl)pyrimidin-5-yl)-2,5-dimethylpiperazine-1-carboxamide was prepared in a similar manner to Description 163 from 2-chloro-4-((2R,5S)-2,5-dimethylpiperazin-1-yl)benzonitrile to give the title compound as a brown solid 176 mg. LCMS (high pH A): Rt=1.08 min, MH⁺=482.1, 484.1.

### Description 164

### 4-(2-((tert-Butyldimethylsilyl)oxy)ethyl)piperidine (D164)

TBDMS-Cl (4.20 g, 27.9 mmol) was added to a solution of 2-(piperidin-4-yl)ethan-1-ol (1.8 g, 13.93 mmol) and imidazole (2.087 g, 30.6 mmol) in DCM (50 mL) at 0° C. and the mixture was stirred for 1 h, then washed with water and the organic layer dried and evaporated in vacuo to give a colourless liquid. The crude was dissolved in DCM and loaded onto a 80 g silica column, then eluted with 0-20% 2 M MeOH ammonia/DCM and product-containing fractions were evaporated in vacuo to give the title compound as a colourless gummy solid (2.78 g, 11.42 mmol, 82% yield).

### Description 165

### 2-(4-(2-((tert-Butyldimethylsilyl)oxy)ethyl)piperidin-1-yl)-5-nitropyridine (D165)

4-(2-((tert-Butyldimethylsilyl)oxy)ethyl)piperidine (may be prepared as described in Description 164; 2.8 g, 11.50 mmol) and DIPEA (3.01 mL, 17.25 mmol) were dissolved in DCM (50 mL) and the solution cooled in an ice bath, then 2-chloro-5-nitropyridine (2.006 g, 12.65 mmol) was added and the mixture stirred at 0° C. for 1 h, then washed with water, the organic layer dried and evaporated in vacuo to give a pale yellow solid. The crude was dissolved in DCM and loaded onto a 80 g silica column and eluted with 0-20% EtOAc/cyclohexane. Product-containing fractions were evaporated in vacuo to give the title compound as a colourless solid (4.6 g, 12.58 mmol). LCMS (high pH A): Rt=1.69 min, MH⁺=366.2.

### Description 166

### 6-(4-(2-((tert-Butyldimethylsilyl)oxy)ethyl)piperidin-1-yl)pyridin-3-amine (D166)

2-(4-(2-((tert-Butyldimethylsilyl)oxy)ethyl)piperidin-1-yl)-5-nitropyridine (may be prepared as described in Description 165; 4.6 g, 12.58 mmol) was dissolved in ethanol (100 mL) and added to a purged hydrogenation flask containing Pd—C 10% (100 mg, 0.940 mmol), then stirred under a hydrogen atmosphere for 2 h. The mixture was filtered under nitrogen and the filtrate evaporated in vacuo. The crude was dissolved in DCM and loaded onto an 80 g silica column, then eluted with 0-100% EtOAc/cyclohexane and product-containing fractions were evaporated in vacuo to give the title compound as a brown gum (2.56 g, 7.63 mmol, 61% yield). LCMS (high pH A): Rt=1.45 min, MH⁺=336.2.

### Description 167

### Phenyl (6-(4-(2-((tert-butyldimethylsilyl)oxy)ethyl)piperidin-1-yl)pyridin-3-yl)carbamate (D167)

6-(4-(2-((tert-Butyldimethylsilyl)oxy)ethyl)piperidin-1-yl)pyridin-3-amine (may be prepared as described in Description 166; 1.75 g, 5.22 mmol) was dissolved in DCM (30 mL) and cooled in an ice bath, then Phenyl chloroformate (0.851 mL, 6.78 mmol) and DIPEA (1.822 mL, 10.43 mmol) were added dropwise and the mixture stirred for 1 h, then washed with water and the organics dried and evaporated in vacuo, The crude was dissolved in DCM and loaded onto a 40 g silica column and eluted with 0-50% EtOAc cyclohexane and product-containing fractions evaporated in vacuo to give the title compound as a pale pink solid (1.51 g, 3.31 mmol, 64% yield). LCMS (high pH A): Rt=1.31 min, MH⁺=456.1.

### Description 168

### (2R,5S)—N-(6-(4-(2-((tert-Butyldimethylsilyl)oxy)ethyl)piperidin-1-yl)pyridin-3-yl)-4-(3-chloro-4-cyanophenyl)-2,5-dimethylpiperazine-1-carboxamide (D168)

2-Chloro-4-((2S,5R)-2,5-dimethylpiperazin-1-yl)benzonitrile (may be prepared as described in Description 142) (247 mg, 0.988 mmol) and 6-(4-(2-((tert-butyldimethylsilyl)oxy)ethyl)piperidin-1-yl)pyridin-3-amine (may be prepared as described in Description 166; 450 mg, 0.988 mmol) were dissolved in MeCN (10 mL) and heated at 60° C. for 2 h, then the solvent was evaporated in vacuo to give a brown gum. The crude was dissolved in DCM and loaded onto a 24 g silica column, then eluted with 0-100% EtOAc/cyclohexane and product-containing fractions evaporated in vacuo to give the title compound as a colourless gum (0.51 g, 0.834 mmol, 84% yield). LCMS (high pH A): Rt=1.65 min, MH⁺=611.2, 613.2.

### Description 169

### (2R,5S)-4-(3-chloro-4-cyanophenyl)-N-(6-(4-(2-hydroxyethyl)piperidin-1-yl)pyridin-3-yl)-2,5-dimethylpiperazine-1-carboxamide (D169)

4 M HCl in dioxane (2 mL, 8.00 mmol) was added to a solution of (2R,5S)—N-(6-(4-(2-((tert-butyldimethylsilyl)oxy)ethyl)piperidin-1-yl)pyridin-3-yl)-4-(3-chloro-4-cyanophenyl)-2,5-dimethylpiperazine-1-carboxamide (may be prepared as described in Description 168; 0.51 g, 0.834 mmol) in DCM (10 mL) at room temperature and the mixture stirred for 30 min then evaporated in vacuo to give a pale yellow foam. The solid was partitioned between aq potassium carbonate and EtOAc and the organic layer dried and evaporated in vacuo to give the title compound as a pale yellow gum (0.35 g, 0.704 mmol, 84% yield). LCMS (high pH A): Rt=1.04 min, MH⁺=497.3, 499.3.

### Description 170

### (2R,5S)-4-(3-chloro-4-cyanophenyl)-2,5-dimethyl-N-(6-(4-(2-oxoethyl)piperidin-1-yl)pyridin-3-yl)piperazine-1-carboxamide (D170)

(2R,5S)-4-(3-chloro-4-cyanophenyl)-N-(6-(4-(2-hydroxyethyl)piperidin-1-yl)pyridin-3-yl)-2,5-dimethylpiperazine-1-carboxamide (may be prepared as described in Description 169; 0.35 g, 0.704 mmol) was dissolved in DCM (20 mL) and cooled in an ice bath and Dess-Martin periodinane (0.388 g, 0.915 mmol) was added, then the mixture stirred at room temperature for 2 h, then evaporated in vacuo to give a gummy solid. The crude was suspended in DCM and loaded onto a 24 g silica column, then eluted with 0-100% EtOAc/cyclohexane and product-containing fractions evaporated in vacuo to give the title compound as a colourless foam (161 mg, 0.325 mmol, 46% yield). LCMS (high pH A): Rt=1.12 min, MH⁺=495.3, 497.3.

### Description 171

### 1-(1-((1-(6-(((1r,4r)-4-(3-Chloro-4-cyanophenoxy)cyclohexyl)carbamoyl)pyridazin-3-yl)piperidin-4-yl)methyl)piperidin-4-yl)-4-(2,4-dioxo-3-((2-(trimethylsilyl)ethoxy)methyl)tetrahydropyrimidin-1(2H)-yl)-N,N-dimethyl-1H-indole-6-carboxamide (D171)

A mixture of 4-(2,4-dioxo-3-((2-(trimethylsilyl)ethoxy)methyl)tetrahydropyrimidin-1(2H)-yl)-N,N-dimethyl-1-(piperidin-4-yl)-1H-indole-6-carboxamide (may be prepared as described in Description 19; 65 mg, 0.127 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 71 mg, 0.152 mmol) in DCM (1.0 mL) was stirred in a stoppered vessel at rom temperature for 10 min. Sodium triacetoxyborohydride (80 mg, 0.380 mmol) was added and the reaction mixture stirred in a stoppered vessel at room temperature for 5 h. The reaction mixture was washed sequentially with saturated NaHCO₃ (aq) (1.0 mL) and brine (1.0 mL), and the organic layer passed through a hydrophobic frit. The filtrate was evaporated in vacuo and the residue loaded in DCM (2 mL) and purified on a 12 g silica cartridge using a gradient of 0-12.5% MeOH in DCM over 14 column volumes. The appropriate fractions were combined and the solvent evaporated in vacuo. The residue was purified by MDAP (high pH). The appropriate fractions were combined and the solvent evaporated in vacuo. The solid was dried in a vacuum oven to give the title compound as a colourless gum (41 mg, 0.042 mmol). LCMS (high pH A): Rt=1.43 min; MH⁺=965, 966, 967.

### Description 172

### tert-Butyl 4-(4-bromo-7H-pyrrolo[2,3-d]pyrimidin-7-yl)piperidine-1-carboxylate (D172)

4-Bromo-7H-pyrrolo[2,3-d]pyrimidine (2 g, 10.10 mmol), Ph₃P (5.30 g, 20.20 mmol), tert-butyl 4-hydroxypiperidine-1-carboxylate (6.10 g, 30.3 mmol) and THF (50 mL) were cooled to 0° C. and treated dropwise with diisopropyl azodicarboxylate (3.93 mL, 20.20 mmol) in THF (10 mL). The mixture was allowed to warm to room temperature overnight. The reaction mixture was evaporated in vacuo and purified by flash chromatography on 330 g silica eluting in 0-100% EtOAc in cyclohexane. Evaporation of the fractions gave the title compound as a thick colourless gum (3.5 g, 8.26 mmol, 82% yield). LCMS (high pH A): Rt=1.24 min, MH⁺=381, 383.

### Description 173

### tert-Butyl 4-(4-(2,4-dioxo-3-((2-(trimethylsilyl)ethoxy)methyl)tetrahydropyrimidin-1(2H)-yl)-7H-pyrrolo[2,3-d]pyrimidin-7-yl)piperidine-1-carboxylate (D173)

3-((2-(Trimethylsilyl)ethoxy)methyl)dihydropyrimidine-2,4(1H,3H)-dione (may be prepared as described in Description 1; 1.666 g, 6.82 mmol), tert-butyl 4-(4-bromo-7H-pyrrolo[2,3-d]pyrimidin-7-yl)piperidine-1-carboxylate (may be prepared as described in Description 172; 2 g, 5.25 mmol), potassium carbonate (1.450 g, 10.49 mmol), trans-N,N′-dimethylcyclohexane-1,2-diamine (0.248 mL, 1.574 mmol), copper(I) iodide (0.150 g, 0.787 mmol) and anhydrous 1,4-dioxane (12 mL) were mixed, degassed with vacuum/N₂ several times, then heated at 135° C. in a sealed tube overnight (ca 16 h). The reaction mixture was cooled, diluted with water and extracted with EtOAc. The organic phase was washed with brine twice, dried, filtered and evaporated in vacuo. The material was purified by flash chromatography on a 80 g silica cartridge eluting in 20-100% EtOAc in cyclohexane. Evaporation of the fractions gave the title compound as a white collapsed foam (1.08 g, 1.884 mmol, 36% yield). LCMS (high pH A): Rt=1.37 min, MH⁺=545.

### Description 174

### Methyl 8-((4-(benzo[d]thiazol-5-ylamino)-6-(tert-butylsulfonyl)quinolin-7-yl)oxy)octanoate (D174)

To a suspension of 4-(benzo[d]thiazol-5-ylamino)-6-(tert-butylsulfonyl)quinolin-7-ol (may be prepared as described in WO2016172134; 8 g, 18.77 mmol) and K₂CO₃ (3.89 g, 28.1 mmol) in DMF (120 mL) stirred under nitrogen at 0° C. was added a solution of methyl 8-bromooctanoate (4.45 g, 18.77 mmol) in DMF (10 mL) dropwise during 5 min. The reaction mixture was stirred at 50° C. for 32 h. The reaction mixture was diluted water (300 mL) and extracted with ethyl acetate (2×250 mL). The combined organic layers were washed with brine (2×200 mL), dried over anhydrous sodium sulphate (15 g), filtered and concentrated under vacuum to get 38 g crude as a yellow oil. The crude residue was dissolved in DCM (200 mL) and absorbed with silica gel (60-120 mesh, 300 g). This was purified by flash column chromatography (Biotage Isolera, 330 g silica gel cartridge, Flow rate: 50 ml/min) using 0-10% methanol in DCM as an eluent over a period of 2 h. The appropriate fractions were concentrated under reduced pressure to give 12 g of a pale yellow gum. This was triturated with diethyl ether and n-hexane (2×200 mL) to obtain a solid. This was filtered through buchner funnel, washed with n-hexane (100 mL) and dried under vacuum to afford the title compound as a pale yellow solid (7.1 g, 12.31 mmol, 66% yield). LCMS (formic B): Rt=1.69 mins, MH⁺=570.

### Description 175

### 8-((4-(Benzo[d]thiazol-5-ylamino)-6-(tert-butylsulfonyl)quinolin-7-yl)oxy)octanoic acid (D175)

To a suspension of methyl 8-((4-(benzo[d]thiazol-5-ylamino)-6-(tert-butylsulfonyl)quinolin-7-yl)oxy)octanoate (may be prepared as described in Description 175; 7.1 g, 12.31 mmol) in MeOH (70 mL), THF (46.7 mL) and water (23.33 mL) stirred at 20° C. was added solid lithium hydroxide hydrate (1.033 g, 24.62 mmol) in one charge. The reaction mixture was stirred at room temperature for 16 h. The reaction mixture was distilled off under vacuum to obtain crude residue. The crude residue was diluted with cold water (100 mL), extracted with EtOAc (100 mL), separated and the organic layer discarded. The aqueous layer was neutralised with 1 N HCl to obtain a solid. The resulting solid was filtered through a buchner funnel, washed with water (100 mL) followed by diethylether (100 mL), and dried under vacuum to give the title compound as an off-white solid (6.455 g, 11.60 mmol, 94% yield). LCMS (formic A): Rt=0.76 mins, MH⁺=556.

### Description 176

### Methyl 5-(1-(3-hydroxypropyl)piperidin-4-yl)pyrazine-2-carboxylate (D176)

To a suspension of methyl 5-(piperidin-4-yl)pyrazine-2-carboxylate (may be prepared as described in WO2008075068; 20.0 g, 90 mmol) in acetonitrile (200 mL) stirred under nitrogen at 0° C. was added triethylamine (37.8 mL, 271 mmol) dropwise, followed by 3-bromopropan-1-ol (12.56 g, 90 mmol). The reaction mixture was stirred at 80° C. for 16 h then concentrated under reduced pressure. To the residue was added water (150 mL) which was extracted with 10% methanol in DCM (2×200 mL). The combined organic layers were washed with brine solution (2×150 mL) and the organic layer was dried over anhydrous Na₂SO₄ (40.0 g), filtered and concentrated under reduced pressure to give crude product as brown solid. The crude material was adsorbed onto 60-120 silica gel (50.0 g), and purified on a 340 g silica cartridge eluting with 0-14% methanol in DCM using a 50 mL/min flow rate. Fractions were collected and concentrated under reduced pressure to give the title compound as an off-white solid (16 g, 55.6 mmol, 62% yield). LCMS (formic B): Rt=0.70 min, MH⁺=280

### Description 177

### Methyl 5-(1-(3-((methylsulfonyl)oxy)propyl)piperidin-4-yl)pyrazine-2-carboxylate (D177)

To a stirred solution of methyl 5-(1-(3-hydroxypropyl)piperidin-4-yl)pyrazine-2-carboxylate (may be prepared as described in Description 176; 10 g, 35.8 mmol) in DCM (100 mL) under nitrogen at −30° C. was added triethylamine (14.97 mL, 107 mmol) and the mixture stirred for 10 min. Methanesulfonyl chloride (3.35 mL, 43.0 mmol) was added dropwise. The reaction mixture was stirred at the same temperature for 30 min. The reaction mixture was concentrated under reduced pressure. To this crude material was added DMF (100 mL), the solids filtered, and the filtrate immediately in the next step. LCMS (formic B) Rt=0.88 min, MH+=358

### Description 178

### Methyl 5-(1-(3-((4-(benzo[d]thiazol-5-ylamino)-6-(tert-butylsulfonyl)quinolin-7-yl)oxy)propyl)piperidin-4-yl)pyrazine-2-carboxylate (D178)

A suspension of 4-(benzo[d]thiazol-5-ylamino)-6-(tert-butylsulfonyl)quinolin-7-ol (may be prepared as described in WO2016172134; 12.18 g, 88 mmol) in DMF (80 mL) was stirred at 70° C. for 30 min and cooled to room temperature. A solution of methyl 5-(1-(3-((methylsulfonyl)oxy)propyl)piperidin-4-yl)pyrazine-2-carboxylate (may be prepared as described in Description 177; 10.5 g, 29.4 mmol) in DMF (100 mL) at 0° C., was added dropwise during 15 min. The reaction mixture was stirred at 70° C. for 1.5 h. The reaction mixture was diluted with ice-cooled water (100 mL) and extracted with EtOAc (2×150 mL). The EtOAc layer was washed with ice cooled water (2×100 mL), saturated sodium chloride solution (2×120 mL), dried over anhydrous sodium sulfate (15.0 g) and concentrated under reduced pressure to give a pale yellow solid. The crude material was adsorbed to 60-120 silica gel (1.0 g) and purified on a 340 g silica cartridge eluting in 0-15% methanol in DCM using a 40 mL/min flow rate. The appropriate fractions were collected and concentrated under reduced pressure to give the title compound as a yellow solid (5.7 g, 8.21 mmol, 28% yield). LCMS (TFA B): Rt=3.86 min, MH⁺=675

### Description 179

### 5-(1-(3-((4-(Benzo[d]thiazol-5-ylamino)-6-(tert-butylsulfonyl)quinolin-7-yl)oxy)propyl)piperidin-4-yl)pyrazine-2-carboxylic acid dihydrochloride (D179)

To a solution of methyl 5-(1-(3-((4-(benzo[d]thiazol-5-ylamino)-6-(tert-butylsulfonyl)quinolin-7-yl)oxy)propyl)piperidin-4-yl)pyrazine-2-carboxylate (may be prepared as described in Description 178; 16.0 g, 23.71 mmol) in THF (500 mL) and MeOH (55 mL) stirred at 0° C. was added a solution of LiOH·H₂O (2.98 g, 71.1 mmol) in water (250 mL) dropwise. The reaction mixture was stirred at room temperature for 3 h. The reaction mixture was concentrated under vacuum. The resulting solution was diluted with water (500 mL) and washed with diethyl ether (2×300 mL) and ethyl acetate (2×200 mL). The aqueous phase was acidified to pH˜4 with 1.5 N HCl solution at −10° C. and the formed solids were filtered through a funnel, and washed with water, diethyl ether and n-hexane. The material was dried under high vacuum to give a yellow solid (14.0 g). This was taken in acetonitrile (500 mL) and stirred at 70° C. for 40 min. After cooling to room temperature, the solids were filtered through a funnel and washed with acetonitrile (2×100 mL). The material was dried under high vacuum, dissolved in acetonitrile:water (1:2) and freeze dried for 20 h to give the title compound as a yellow solid (13.2 g, 17.46 mmol, 74% yield). LCMS (formic B): Rt=1.14 min. MH+=661

### Description 180

### 2-(2-(2-(2-((4-(Benzo[d]thiazol-5-ylamino)-6-(tert-butylsulfonyl)quinolin-7-yl)oxy)ethoxy)ethoxy)ethyl) isoindoline-1,3-dione (D180)

To a stirred solution of 4-(benzo[d]thiazol-5-ylamino)-6-(tert-butylsulfonyl)quinolin-7-ol (may be prepared as described in WO2016172134; 5.00 g, 12.09 mmol) in DMF (80 mL) was added 2-(2-(2-(2-chloroethoxy)ethoxy)ethyl) isoindoline-1,3-dione (synthesis described by Lukyanenko, N. G. et al. Journal of the Chemical Society, Perkin Transactions 1, 2002, 21, 2347-2351; 5.40 g, 18.14 mmol) and potassium carbonate (5.01 g, 36.3 mmol) at room temperature. The reaction mixture was heated 90° C. for 16 h. The reaction mixture was diluted with ethyl acetate (50 mL) and filtered through a celite bed. The celite bed was washed with ethyl acetate (3×100 mL)) and concentrated under reduced pressure. The crude was absorbed into silica gel 2 g (230-400 mesh) and purified by on a 40 g silica cartridge using 6% MeOH in DCM as eluent to give the title compound as a yellow solid (6.0 g, 3.54 mmol, 29% yield). LCMS (formic B): Rt=1.55 min, MH+=675

### Description 181

### N-(7-(2-(2-(2-Aminoethoxy)ethoxy)ethoxy)-6-(tert-butylsulfonyl)quinolin-4-yl)benzo[d]thiazol-5-amine (D181)

To a stirred solution of 2-(2-(2-(2-((4-(benzo[d]thiazol-5-ylamino)-6-(tert-butylsulfonyl)quinolin-7-yl)oxy)ethoxy)ethoxy)ethyl) isoindoline-1,3-dione (may be prepared as described in Description 180; 6 g, 8.89 mmol) in ethanol (50 mL) was added hydrazine hydrate (0.524 mL, 10.67 mmol). The reaction mixture was stirred at room temperature for 16 h. The reaction mixture was concentrated under reduced pressure. The crude was diluted with water (50 mL) and the organic layer was extracted with 10% methanol in DCM (3×100 mL). The combined organic layers were washed with water (50 mL) and brine solution (50 mL), dried over anhydrous Na₂SO₄ (1 g) and concentrated under reduced pressure. The crude was absorbed into silica gel 2 g (230-400 mesh) and purified on a 40 g silica cartridge using 15% MeOH in DCM with triethylamine to give the title compound as a yellow solid (700 mg, 1.285 mmol, 14% yield). LCMS (high pH B): Rt=2.41 min, MH+=545.2

### Description 182

### tert-Butyl (4-(4-((1-(4-((2-(2-(2-azidoethoxy)ethoxy)ethyl)(methyl)carbamoyl)phenyl)-3-(difluoromethyl)-1H-pyrazol-4-yl)carbamoyl)oxazol-2-yl)pyridin-2-yl)(cyclopropylmethyl)carbamate (D182)

To a suspension of 4-(4-(2-(2-((tert-butoxycarbonyl) (cyclopropylmethyl)amino)pyridin-4-yl)oxazole-4-carboxamido)-3-(difluoromethyl)-1H-pyrazol-1-yl)benzoic acid (may be prepared as described in US20190192668; 798 mg, 1.342 mmol) in anhydrous DCM (10 mL) and DIPEA (0.740 mL, 4.24 mmol) was added HATU (806 mg, 2.120 mmol). The suspension was stirred at ambient temperature for 5 min, and a solution of 2-(2-(2-(methylamino)ethoxy)ethoxy)ethan-1-ol (266 mg, 1.413 mmol) in anhydrous DCM (3 mL) was added. The reaction mixture was stirred at ambient temperature for 18 h 20 min. The reaction mixture was partitioned between DCM (10 mL) and saturated aqueous sodium hydrogen carbonate solution (20 mL). The organic layer was passed through a hydrophobic frit and evaporated in vacuo to yield an orange oil which was stood at ambient temperature for 19 days (convenience). This was dissolved in DCM (10 mL), applied to a 80 g silica column and eluted with a gradient of 3:1 ethyl acetate:ethanol (0-100%) in cyclohexane over 60 min (flow rate=60 mL/min). The appropriate fractions were combined and evaporated in vacuo to yield the title compound as a pale yellow oil (823 mg, 1.076 mmol, 76% yield). LCMS (high pH A): Rt=1.50 min MH+765/766

### Description 183

### tert-Butyl (4-(4-((1-(4-((2-(2-(2-aminoethoxy)ethoxy)ethyl)(methyl)carbamoyl)phenyl)-3-(difluoromethyl)-1H-pyrazol-4-yl)carbamoyl)oxazol-2-yl)pyridin-2-yl)(cyclopropylmethyl)carbamate (D183)

tert-Butyl (4-(4-((1-(4-((2-(2-(2-azidoethoxy)ethoxy)ethyl)(methyl)carbamoyl)phenyl)-3-(difluoromethyl)-1H-pyrazol-4-yl)carbamoyl)oxazol-2-yl)pyridin-2-yl) (cyclopropylmethyl)carbamate (may be prepared as described in Description 182; 60 mg, 0.078 mmol) dissolved in THF (0.5 mL) and water (0.1 mL) was treated with PPh₃ (61.7 mg, 0.235 mmol) and heated at 55° C. overnight (approximately 18 h). The reaction mixture was blown down and purified by flash chromatography on 12 g silica eluting with 0-7% 4 M NH₃-MeOH in DCM to give the title compound as a colourless sticky collapsed foam after evaporation in vacuo (46 mg, 0.059 mmol, 75% yield). LCMS (formic A): Rt=0.98 min, MH+739

### Description 184

### Benzyl 4-(4-bromo-1H-indazol-1-yl)piperidine-1-carboxylate (D184)

4-Bromo-1H-indazole (2 g, 10.15 mmol) and DMF (20 mL) were mixed, stirred at room temperature and treated with 60% NaH (0.812 g, 20.30 mmol). This was stirred for 15 min, then treated with benzyl 4-((methylsulfonyl)oxy)piperidine-1-carboxylate (9.54 g, 30.5 mmol) in approximately three batches at hourly intervals. The reaction mixture was diluted with ether and NH₄Cl, washed with brine, dried, filtered and evaporated in vacuo. Flash chromatography on silica (120 g) eluting with 0-50% EtOAc/cyclohexane and evaporation in vacuo gave the title compound as a colourless oil (1.04 g, 2.385 mmol, 23% yield). LCMS (high pH): Rt=1.40 min, MH⁺=414, 416.

### Description 185

### Benzyl 4-(4-(2,4-dioxo-3-((2-(trimethylsilyl)ethoxy)methyl)tetrahydropyrimidin-1(2H)-yl)-1H-indazol-1-yl)piperidine-1-carboxylate (D185)

A mixture of copper(I) iodide (41 mg, 0.215 mmol), potassium carbonate (588 mg, 4.26 mmol) and 3-((2-(trimethylsilyl)ethoxy)methyl)dihydropyrimidine-2,4(1H,3H)-dione (598 mg, 2.447 mmol) was diluted with a solution of benzyl 4-(4-bromo-1H-indazol-1-yl)piperidine-1-carboxylate (may be prepared as described in Description 184; 882 mg, 2.129 mmol) in anhydrous 1,4-dioxane (11 mL). Trans-N,N′-Dimethylcyclohexane-1,2-diamine (0.067 mL, 0.426 mmol) was added, and the vessel evacuated and purged with nitrogen (×3). The mixture was stirred at 140° C. for 6 h under nitrogen. Copper(I) iodide (41 mg) and trans-N,N′-Dimethylcyclohexane-1,2-diamine (0.067 mL were added and the mixture stirred at 140° C. for 16 h. The reaction was allowed to cool to room temperature, the suspension filtered through Celite and the Celite washed with EtOAc (20 mL). The filtrate was evaporated in vacuo and the residue loaded in DCM (3 mL) and purified on a 40 g silica cartridge using a gradient of 0-5% MeOH in DCM over 14 column volumes. The appropriate fractions were combined and the solvent evaporated in vacuo to give the title compound as an off white gum (1.35 g, 2.337 mmol) which contained EtOAc by ¹H NMR. LCMS (high pH): Rt=1.31 min, MH⁺=578.

### Description 186

### tert-Butyl ((1r,3r)-3-(4-cyano-3-(trifluoromethyl)phenoxy)-2,2,4,4-tetramethylcyclobutyl)carbamate (D186)

Under an atmosphere of nitrogen an ice-cooled solution of tert-butyl ((1r,3r)-3-hydroxy-2,2,4,4-tetramethylcyclobutyl)carbamate (620 mg, 2.55 mmol) in anhydrous DMF (10 mL) was treated with 60% sodium hydride (153 mg, 3.82 mmol) and stirred with cooling for 15 min. 4-fluoro-2-(trifluoromethyl)benzonitrile (530 mg, 2.80 mmol) was added and the mixture was stirred with cooling for 1 h then treated cautiously with 10% aqueous ammonium chloride (20 mL). The mixture was extracted with EtOAc (2×40 mL), the combined organics were washed with 5% aqueous lithium chloride (3×40 mL), dried (MgSO₄), filtered and evaporated to dryness. The product was purified by chromatography on silica using a gradient elution from 0% to 50% ethyl acetate in cyclohexane to give the title compound (1.01 g, 2.449 mmol, 96% yield). LCMS (formic A): Rt=1.47 min, M-^{t}Bu⁺=357.

### Description 187

### tert-Butyl (cyclopropylmethyl)(4-(4-((3-(difluoromethyl)-1-(4-((2-(2-(2-(4-(2,4-dioxotetrahydropyrimidin-1(2H)-yl)-1-isopropyl-1H-indole-6-carboxamido)ethoxy)ethoxy)ethyl)(methyl)carbamoyl)phenyl)-1H-pyrazol-4-yl)carbamoyl)oxazol-2-yl)pyridin-2-yl)carbamate (D187)

4-(2,4-Dioxotetrahydropyrimidin-1(2H)-yl)-1-isopropyl-1H-indole-6-carboxylic acid (may be prepared as described in Description 35; 15 mg, 0.048 mmol), HATU (23.51 mg, 0.062 mmol), DMF (0.5 mL) and DIPEA (0.025 mL, 0.143 mmol) were mixed and stirred for 10 min, then treated with tert-butyl (4-(4-((1-(4-((2-(2-(2-aminoethoxy)ethoxy)ethyl)(methyl)carbamoyl)phenyl)-3-(difluoromethyl)-1H-pyrazol-4-yl)carbamoyl)oxazol-2-yl)pyridin-2-yl)(cyclopropylmethyl)carbamate (may be prepared as described in Description 183; 35.1 mg, 0.048 mmol) and allowed to stir overnight (approximately 18 h). The reaction mixture was blown down slightly then diluted to 1 mL with DMSO-methanol and purified by MDAP (high pH). Freeze-drying overnight (approximately 20 h) gave the title compound as a fluffy white powder (25 mg, 0.023 mmol, 48% yield). LCMS (high pH A): Rt=1.15 min, MH+=936

### Description 188

### Benzyl 4-(4-bromo-1H-pyrazolo[3,4-b]pyridin-1-yl)piperidine-1-carboxylate (D188)

4-Bromo-1H-pyrazolo[3,4-b]pyridine (3 g, 15.15 mmol) in DMF (30 mL) was stirred at room temperature, treated with NaH (1.212 g, 30.3 mmol), stirred 15 min, treated with one-third of the portion of benzyl 4-((methylsulfonyl)oxy)piperidine-1-carboxylate (14.24 g, 45.4 mmol) and heated to 80° C. At approximately 1 h intervals, the remaining ⅔ benzyl 4-((methylsulfonyl)oxy)piperidine-1-carboxylate was added. After 4 h the reaction was allowed to cool overnight (about 16 h) and allowed to stand for about two weeks. The reaction was diluted with ethyl acetate and NH₄Cl, washed with brine (×2), dried, filtered, evaporated in vacuo and purified by flash chromatography (120 g, silica, 0-70% ethyl acetate in cyclohexane) to give benzyl 4-(4-bromo-1H-pyrazolo[3,4-b]pyridin-1-yl)piperidine-1-carboxylate (1.3 g, 2.82 mmol, 18.60% yield). LCMS (high pH A): Rt=1.34 min, MH⁺=417

### Description 189

### Benzyl 4-(4-(2,4-dioxotetrahydropyrimidin-1(2H)-yl)-1H-pyrazolo[3,4-b]pyridin-1-yl)piperidine-1-carboxylate (D189)

3-((2-(Trimethylsilyl)ethoxy)methyl)dihydropyrimidine-2,4(1H,3H)-dione (may be prepared as described in Description 1; 0.994 g, 4.07 mmol), benzyl 4-(4-bromo-1H-pyrazolo[3,4-b]pyridin-1-yl)piperidine-1-carboxylate (may be prepared as described in Description 188; 1.3 g, 3.13 mmol), potassium carbonate (0.865 g, 6.26 mmol), trans-N,N′-dimethylcyclohexane-1,2-diamine (0.148 mL, 0.939 mmol) and copper(I) iodide (0.089 g, 0.470 mmol) in dioxane (13 mL) were mixed, degassed with vacuum/N₂ several times, then heated at 135° C. in a sealed tube overnight (approximately 16 h). The reaction was cooled, diluted with water, extracted with ethyl acetate, washed with brine twice, dried, filtered and evaporated in vacuo to give a pale brown oil which was taken up in DCM (5.00 mL), treated with TFA (10 mL, 130 mmol) and stirred for 2 h. The reaction was blown down under nitrogen, taken up in 4M NH₃-methanol, stirred 1 h, blown down and allowed to stand overnight to give a white solid. The mixture was diluted with water and ethyl acetate (solid did not dissolve). The ethyl acetate was evaporated off, and the compound filtered off from the water and dried by suction to give crude benzyl 4-(4-(2,4-dioxotetrahydropyrimidin-1(2H)-yl)-1H-pyrazolo[3,4-b]pyridin-1-yl)piperidine-1-carboxylate (1.2 g, 2.408 mmol, 77% yield). LCMS (high pH A): Rt=0.94 min, MH⁺=449.2

### Description 190

### N-((1r,4S)-4-(3-Chloro-4-cyanophenoxy)cyclohexyl)-6-((S)-2-(hydroxymethyl)morpholino)pyridazine-3-carboxamide (D190)

6-Chloro-N-((1r,4r)-4-(3-chloro-4-cyanophenoxy)cyclohexyl)pyridazine-3-carboxamide (may be prepared as described in Description 90; 199 mg, 0.509 mmol), (S)-morpholin-2-ylmethanol hydrochloride (94 mg, 0.610 mmol) and DIPEA (0.222 ml, 1.272 mmol) were added to a microwave vial to which was added DMSO (2 ml), and the mixture was heated at 70° C. for 16 h, diluted with water (20 mL) and extracted with ethyl acetate (20 mL). The organics were washed with water (20 mL), filtered through a hydrophobic frit and concentrated in vacuo to give an orange-coloured gum. This was dissolved in minimum DCM and loaded onto a 24 g silica column, then eluted with 0-20% ethanol in ethyl acetate to give N-((1r,4S)-4-(3-chloro-4-cyanophenoxy)cyclohexyl)-6-((S)-2-(hydroxymethyl)morpholino)pyridazine-3-carboxamide (204 mg, 0.432 mmol, 85% yield). LCMS (high pH A): Rt=1.01 min, MH⁺=472, 474

### Description 191

### N-((1r,4S)-4-(3-Chloro-4-cyanophenoxy)cyclohexyl)-6-((S)-2-formylmorpholino)pyridazine-3-carboxamide (D191)

Dess-Martin periodinane (220 mg, 0.519 mmol) was added to a solution of N-((1r,4S)-4-(3-chloro-4-cyanophenoxy)cyclohexyl)-6-((S)-2-(hydroxymethyl)morpholino)pyridazine-3-carboxamide (may be prepared as described in Description 190; 200 mg, 0.424 mmol) in DCM (20 mL) and the mixture was stirred at room temperature for 2 h, quenched with saturated aq sodium thiosulphate (2 ml), stirred for 10 min, then stirred with saturated sodium bicarbonate (10 ml) for 10 minutes. The organic layer was separated, dried and evaporated in vacuo to give N-((1r,4S)-4-(3-chloro-4-cyanophenoxy)cyclohexyl)-6-((S)-2-formylmorpholino)pyridazine-3-carboxamide (185 mg, 0.394 mmol, 93% yield). LCMS (high pH A): Rt=0.96 min, M+H₂O⁺=488, 490

### Description 192

### N-((1r,4R)-4-(3-Chloro-4-cyanophenoxy)cyclohexyl)-6-((R)-2-(hydroxymethyl)morpholino)pyridazine-3-carboxamide (D192)

6-Chloro-N-((1r,4r)-4-(3-chloro-4-cyanophenoxy)cyclohexyl)pyridazine-3-carboxamide (may be prepared as described in Description 90; 208 mg, 0.532 mmol), (R)-morpholin-2-ylmethanol hydrochloride (98 mg, 0.638 mmol) and DIPEA (0.232 ml, 1.329 mmol) were added to a microwave vial to which was added DMSO (2 ml), and the mixture heated at 70° C. for 16 h. The mixture was diluted with water (20 mL) and extracted with ethyl acetate (20 mL). The organics were washed with water (20 mL), filtered through a hydrophobic frit and concentrated in vacuo to give an orange-coloured gum. This was dissolved in minimum DCM and loaded onto a 24 g silica column, then eluted with 0-20% ethanol in ethyl acetate to give N-((1r,4R)-4-(3-chloro-4-cyanophenoxy)cyclohexyl)-6-((R)-2-(hydroxymethyl)morpholino)pyridazine-3-carboxamide (257 mg, 0.545 mmol, 102% yield). LCMS (high pH A): Rt=1.02 min, MH⁺=472, 474

### Description 193

### N-((1r,4R)-4-(3-chloro-4-cyanophenoxy)cyclohexyl)-6-((R)-2-formylmorpholino)pyridazine-3-carboxamide (D193)

Dess-Martin periodinane (300 mg, 0.707 mmol) was added to a solution of N-((1r,4S)-4-(3-chloro-4-cyanophenoxy)cyclohexyl)-6-((S)-2-(hydroxymethyl)morpholino)pyridazine-3-carboxamide (may be prepared as described in Description 192; 250 mg, 0.530 mmol) in DCM (20 mL) and the mixture was stirred at room temperature for 2 h, quenched with saturated aq sodium thiosulphate (2 ml), stirred for 10 min, then stirred with saturated sodium bicarbonate (10 ml) for 10 minutes. The organic layer was separated, evaporated in vacuo to give N-((1r,4R)-4-(3-chloro-4-cyanophenoxy)cyclohexyl)-6-((R)-2-formylmorpholino)pyridazine-3-carboxamide (251 mg, 0.534 mmol, 101% yield). LCMS (high pH A): Rt=0.96 min, M+H₂O⁺=488, 490

### Description 194

### N-((1r,4r)-4-(3-chloro-4-cyanophenoxy)cyclohexyl)-4-(2-hydroxyethyl)benzamide (D194)

To a stirring solution of 4-(2-hydroxyethyl)benzoic acid (100 mg, 0.602 mmol), 4-(((1r,4r)-4-aminocyclohexyl)oxy)-2-chlorobenzonitrile (may be prepared as described in Description 46; 226 mg, 0.903 mmol) and HATU (275 mg, 0.722 mmol) in DMF (2 mL) was added DIPEA (0.314 mL, 1.805 mmol). After 17 h, the solution was diluted with ethyl acetate (20 mL) and water (20 mL), and the layers were separated. The organic layer was filtered through a hydrophobic frit and concentrated in vacuo. The crude product was dissolved in ethyl acetate and purified using a 24 g silica column, eluting with 0-10% ethanol in ethyl acetate to give N-((1r,4r)-4-(3-chloro-4-cyanophenoxy)cyclohexyl)-4-(2-hydroxyethyl)benzamide (194 mg, 0.486 mmol, 81% yield). LCMS (high pH A): Rt=1.05 min, MH⁺=399, 401

### Description 195

### 4-(((1r,4r)-4-(3-chloro-4-cyanophenoxy)cyclohexyl)carbamoyl)phenethyl 4-methylbenzenesulfonate (D195)

To a stirring solution of N-((1r,4r)-4-(3-chloro-4-cyanophenoxy)cyclohexyl)-4-(2-hydroxyethyl)benzamide (may be prepared as described in Description D194; 194 mg, 0.486 mmol) and tosyl-Cl (278 mg, 1.459 mmol) in DCM (4864 μl) was added triethylamine (271 μl, 1.945 mmol), followed by stirring for 16 h. This was then left to stand for 10 days. The pale yellow mixture was diluted with EtOAc (100 mL) and water (50 mL). The layers were separated, and the aqueous layer was extracted with EtOAc (50 mL). The combined organic layers were filtered through a hydrophobic frit and concentrated in vacuo. The crude product was purified using a 24 g silica column, eluting with 0-70% EtOAc to in cyclohexane give 4-(((1r,4r)-4-(3-chloro-4-cyanophenoxy)cyclohexyl)carbamoyl)phenethyl 4-methylbenzenesulfonate (135 mg, 0.244 mmol, 50.2% yield). LCMS (high pH A): Rt=1.33 min, MH⁺=554

### Description 196

### tert-Butyl 3-(4-bromo-1H-indol-1-yl)azetidine-1-carboxylate (D196)

To a solution of 4-bromo-1H-indole (0.26 ml, 2.073 mmol) in DMF (11 ml) stirred under nitrogen at 0° C. was added sodium hydride (0.108 g, 2.69 mmol), portion wise. The reaction mixture was stirred at 0° C. for 5 min. To the reaction mixture was added tert-butyl 3-iodoazetidine-1-carboxylate (0.7 g, 2.473 mmol). The reaction mixture was stirred at 60° C. for 1.5 h and was cooled to room temperature. The reaction was quenched by addition of saturated aqueous ammonium chloride (50 mL), and the mixture was extracted with ethyl acetate (3×50 mL). The organics were combined, washed with 5% aqueous LiCl (4×25 mL), brine (40 mL), dried by passing through a hydrophobic frit, and the solvent removed in vacuo to give crude product, which was dissolved in DCM (1 mL) and loaded onto a cyclohexane preconditioned 40 g Redisep silica column. The crude material on silica was purified by Combiflash using a gradient of 0-30% TBME in cyclohexane to give tert-butyl 3-(4-bromo-1H-indol-1-yl)azetidine-1-carboxylate (364 mg, 1.036 mmol, 50.0% yield). LCMS (high pH A): Rt=1.37 min, MH⁺=350, 352

### Description 197

### tert-Butyl 3-(4-(2,4-dioxo-3-((2-(trimethylsilyl)ethoxy)methyl)tetrahydropyrimidin-1(2H)-yl)-1H-indol-1-yl)azetidine-1-carboxylate (D197)

3-((2-(Trimethylsilyl)ethoxy)methyl)dihydropyrimidine-2,4(1H,3H)-dione (may be prepared as described in Description D1; 288 mg, 1.179 mmol), potassium carbonate (307 mg, 2.218 mmol), copper(I) iodide (30.9 mg, 0.162 mmol), trans-N,N′-dimethylcyclohexane-1,2-diamine (0.051 mL, 0.325 mmol) and a solution of tert-butyl 3-(4-bromo-1H-indol-1-yl)azetidine-1-carboxylate (may be prepared as described in Description 196; 380 mg, 1.082 mmol) in anhydrous 1,4-dioxane (12.5 mL) were mixed in a microwave vial, flushed with nitrogen, sealed and heated at 140° C. for 23 hr. The reaction was cooled to room temperature. The reaction mixture was evaporated in vacuo, diluted with DCM (15 mL) and water (15 mL). The layers were separated, and the aqueous extracted with DCM (15 mL). The organics were combined, dried by passing through a hydrophobic frit, and the solvent removed in vacuo to give the crude material, which was dissolved in minimal DCM and loaded onto a cyclohexane preconditioned 40 g Redisep silica column and purified by Combiflash using a gradient of 0-50% EtOAc in cyclohexane to give tert-butyl 3-(4-(2,4-dioxo-3-((2-(trimethylsilyl)ethoxy)methyl)tetrahydropyrimidin-1(2H)-yl)-1H-indol-1-yl)azetidine-1-carboxylate (430 mg, 0.835 mmol, 77% yield). LCMS (high pH A): Rt=1.36 min, MH⁺=product does not ionize well.

### Description 198

### tert-Butyl (2S,5R)-4-(3-chloro-4-cyanophenyl)-2,5-dimethylpiperazine-1-carboxylate (D198)

A mixture of 2-chloro-4-fluorobenzonitrile (5 g, 32.1 mmol), potassium carbonate (8.88 g, 64.3 mmol) and tert-butyl (2S,5R)-2,5-dimethylpiperazine-1-carboxylate (6.89 g, 32.1 mmol) in DMSO (40 mL) and water (4 mL) was stirred at 60° C. for 46 h. The mixture was allowed to cool to room temperature, diluted with water (100 mL), then extracted with EtOAc (2×100 mL). The organic extracts were combined, washed with water and dried over sodium sulphate. The organics were concentrated under vacuum to give tert-butyl (2S,5R)-4-(3-chloro-4-cyanophenyl)-2,5-dimethylpiperazine-1-carboxylate (7.2 g, 20.58 mmol, 64.0% yield). LCMS (high pH A): Rt=1.34 min, MH⁺=349.9

### Description 199

### 2-Chloro-4-((2R,5S)-2,5-dimethylpiperazin-1-yl)benzonitrile (D199)

4M HCl in dioxane (20 mL, 80 mmol) was added to a solution of tert-butyl (2S,5R)-4-(3-chloro-4-cyanophenyl)-2,5-dimethylpiperazine-1-carboxylate (may be prepared as described in Description 198; 7.2 g, 20.58 mmol) in DCM (40 ml), and the mixture was stirred overnight at room temperature. The resulting suspension was diluted with TBME (50 ml) and filtered to give a hygroscopic solid. The solid was dissolved in water (50 ml) and basified with solid potassium carbonate (10 g), then extracted with EtOAc (2×50 ml). The organics were dried and evaporated in vacuo to give a pale yellow gum which was dissolved in DCM and loaded onto a 120 g silica column, eluting with 0-10% 2M methanolic ammonia in DCM to give 2-chloro-4-((2R,5S)-2,5-dimethylpiperazin-1-yl)benzonitrile (3.5 g, 14.01 mmol, 68.1% yield). LCMS (high pH A): Rt=0.96 min, MH⁺=250.0, 252.0

### Description 200

### (2S,5R)—N-(6-(4-(2-((tert-butyldimethylsilyl)oxy)ethyl)piperidin-1-yl)pyridin-3-yl)-4-(3-chloro-4-cyanophenyl)-2,5-dimethylpiperazine-1-carboxamide (D200)

2-Chloro-4-((2R,5S)-2,5-dimethylpiperazin-1-yl)benzonitrile (may be prepared as described in Description 199; 278 mg, 1.113 mmol) and phenyl (6-(4-(2-((tert-butyldimethylsilyl)oxy)ethyl)piperidin-1-yl)pyridin-3-yl)carbamate (may be prepared as described in Description D167; 461 mg, 1.012 mmol) were dissolved in acetonitrile (10 mL) and stirred at 70° C. for 2.5 h. The temperature was increased to 80° C., and the reaction was stirred for a further 21.5 h. The reaction mixture was left standing for 16 hours. The solvent was evaporated, and the crude residue was dissolved in DCM and chromatographed (0-100% ethyl acetate in cyclohexane) to give (2S,5R)—N-(6-(4-(2-((tert-butyldimethylsilyl)oxy)ethyl)piperidin-1-yl)pyridin-3-yl)-4-(3-chloro-4-cyanophenyl)-2,5-dimethylpiperazine-1-carboxamide (567 mg, 0.928 mmol, 92% yield). LCMS (high pH A): Rt=1.64 min, MH⁺=611.2

### Description 201

### (2S,5R)-4-(3-Chloro-4-cyanophenyl)-N-(6-(4-(2-hydroxyethyl)piperidin-1-yl)pyridin-3-yl)-2,5-dimethylpiperazine-1-carboxamide (D201)

(2S,5R)—N-(6-(4-(2-((tert-Butyldimethylsilyl)oxy)ethyl)piperidin-1-yl)pyridin-3-yl)-4-(3-chloro-4-cyanophenyl)-2,5-dimethylpiperazine-1-carboxamide (may be prepared as described in Description 200; 528 mg, 0.864 mmol) and hydrogen chloride in 1,4-dioxane (2.160 mL, 8.64 mmol, 4M) were stirred in DCM (5 mL) at room temperature for 10 minutes. The solvent was evaporated, and the crude residue was partitioned between saturated sodium bicarbonate (10 mL×2) and ethyl acetate (25 mL×2). The aqueous phase was separated. The organic phase was washed with water (20 mL) and brine (30 mL), dried and evaporated to give (2S,5R)-4-(3-chloro-4-cyanophenyl)-N-(6-(4-(2-hydroxyethyl)piperidin-1-yl)pyridin-3-yl)-2,5-dimethylpiperazine-1-carboxamide (390 mg, 0.785 mmol, 91% yield). LCMS (high pH A): Rt=1.05 min, MH⁺=497.3, 499.2

### Description 202

### (2S,5R)-4-(3-Chloro-4-cyanophenyl)-2,5-dimethyl-N-(6-(4-(2-oxoethyl)piperidin-1-yl)pyridin-3-yl)piperazine-1-carboxamide (D15)

Dimethyl sulfoxide (0.415 mL, 5.85 mmol) was added to oxalyl dichloride (0.248 mL, 2.93 mmol) and 3 Å molecular sieves in DCM (3 mL) at −78° C. under nitrogen. After 30 minutes a solution of (2S,5R)-4-(3-chloro-4-cyanophenyl)-N-(6-(4-(2-hydroxyethyl)piperidin-1-yl)pyridin-3-yl)-2,5-dimethylpiperazine-1-carboxamide (may be prepared as described in Description 201; 291 mg, 0.585 mmol) in DCM (6 mL) was added slowly to the reaction mixture. After 15 minutes, triethylamine (1.224 mL, 8.78 mmol) in DCM (6 mL) was added dropwise. The reaction was stirred for a further 15 minutes and then allowed to slowly warm up to room temperature whilst stirring under nitrogen for 2 hours. The reaction mixture was diluted with water (10 mL), and the organic layer was separated. The aqueous layer was then extracted with DCM (2×5 mL). The combined organic extracts were washed with water (15 mL×2) and brine (15 mL). The organic layer was evaporated and dried to give (2S,5R)-4-(3-chloro-4-cyanophenyl)-2,5-dimethyl-N-(6-(4-(2-oxoethyl)piperidin-1-yl)pyridin-3-yl)piperazine-1-carboxamide (189 mg, 0.382 mmol, 65.2% yield). LCMS (high pH A): Rt=1.12 min, MH⁺=495.3, 497.2

### Description 203

### 2-(4-(6-(((1r,4r)-4-(3-chloro-4-tert-Butyl cyanophenoxy)cyclohexyl)carbamoyl)pyridazin-3-yl)piperazin-1-yl)acetate (D203)

N-((1r,4r)-4-(3-chloro-4-cyanophenoxy)cyclohexyl)-6-(piperazin-1-yl)pyridazine-3-carboxamide, trifluoroacetic acid salt (may be prepared as described in Description 121; 210 mg, 0.378 mmol) and potassium carbonate (131 mg, 0.946 mmol) were suspended in DMF (2 mL). To this mixture was added tert-butyl 2-bromoacetate (0.067 mL, 0.454 mmol), followed by stirring for 16 h. The mixture was diluted with water (20 mL) and EtOAc (20 mL), and they layers were separated. The organic layer was washed with water (20 mL), filtered through a hydrophobic frit and concentrated in vacuo to give tert-butyl 2-(4-(6-(((1r,4r)-4-(3-chloro-4-cyanophenoxy)cyclohexyl)carbamoyl)pyridazin-3-yl)piperazin-1-yl)acetate (203 mg, 0.366 mmol, 97% yield). LCMS (high pH A): Rt=1.29 min, MH⁺=555.0, 557.0

### Description 204

### 2-(4-(6-(((1r,4r)-4-(3-Chloro-4-cyanophenoxy)cyclohexyl)carbamoyl)pyridazin-3-yl)piperazin-1-yl) acetic acid, trifluoroacetic acid salt (D204)

To a stirring solution of tert-butyl 2-(4-(6-(((1r,4r)-4-(3-chloro-4-cyanophenoxy)cyclohexyl)carbamoyl)pyridazin-3-yl)piperazin-1-yl)acetate (may be prepared as described in Description 203; 203 mg, 0.366 mmol) in DCM (5 mL) was added trifluoroacetic acid (0.564 mL, 7.31 mmol), followed by stirring for 4 h. A further portion of trifluoroacetic acid (41.7 mg, 0.366 mmol) was added, followed by stirring for 18 h. The reaction was concentrated using a flow of nitrogen gas to give 2-(4-(6-(((1r,4r)-4-(3-chloro-4-cyanophenoxy)cyclohexyl)carbamoyl)pyridazin-3-yl)piperazin-1-yl) acetic acid, trifluoroacetic acid salt (435 mg, 0.710 mmol, 194% yield [residual TFA present]). LCMS (high pH A): Rt=0.82 min, MH⁺=499.0, 501.0

### Description 205

### N-((1r,4r)-4-(3-Chloro-4-cyanophenoxy)cyclohexyl)-6-(4-(1-hydroxyethyl)piperidin-1-yl)pyridazine-3-carboxamide (D205)

To a stirring solution of 6-chloro-N-((1r,4r)-4-(3-chloro-4-cyanophenoxy)cyclohexyl)pyridazine-3-carboxamide (may be prepared as described in Description 90; 300 mg, 0.767 mmol) and 1-(piperidin-4-yl)ethan-1-ol (119 mg, 0.920 mmol) in DMSO (1501 μl) was added DIPEA (161 μl, 0.920 mmol), and the mixture was heated at 70° C. for 3 h. The mixture was diluted with water (30 mL) and EtOAc (30 mL), and the layers were separated. The aqueous layer was extracted with EtOAc (2×20 mL), and the combined organic layers were filtered through a hydrophobic frit and concentrated in vacuo to give a pale yellow gum. The crude product was dissolved in DCM and loaded onto a 24 g silica column, eluting with 0-100% (25% ethanol/ethyl acetate 1% NH₄OH) in cyclohexane to give N-((1r,4r)-4-(3-chloro-4-cyanophenoxy)cyclohexyl)-6-(4-(1-hydroxyethyl)piperidin-1-yl)pyridazine-3-carboxamide (189 mg, 0.390 mmol, 50.9% yield). LCMS (high pH A): Rt=1.12 min, MH⁺=484.0, 486.0

### Description 206

### 6-(4-Acetylpiperidin-1-yl)-N-((1r,4r)-4-(3-chloro-4-cyanophenoxy)cyclohexyl)pyridazine-3-carboxamide (D206)

A mixture of N-((1r,4r)-4-(3-chloro-4-cyanophenoxy)cyclohexyl)-6-(4-(1-hydroxyethyl)piperidin-1-yl)pyridazine-3-carboxamide (may be prepared as described in Description 205; 87 mg, 0.180 mmol) and Dess-Martin periodinane (159 mg, 0.375 mmol) in DCM (3900 μl) was stirred at room temperature for 1 h. Sodium thiosulfate solution (3 mL) was added, and the reaction mixture was stirred for 10 minutes. The solution was diluted with sodium bicarbonate solution (10 mL) and DCM (10 mL), and the layers were separated. The organic layer was filtered through a hydrophobic frit and evaporated to dryness in air. The crude product was dissolved in a minimal volume of DCM and purified using a 12 g silica column, eluting with 0-100% EtOAc in cyclohexane to give 6-(4-acetylpiperidin-1-yl)-N-((1r,4r)-4-(3-chloro-4-cyanophenoxy)cyclohexyl)pyridazine-3-carboxamide (60 mg, 0.124 mmol, 69.3% yield). LCMS (high pH A): Rt=1.15 min, MH⁺=482.0, 484.0

### Description 207

### (2S,5R)—N-(6-(4-(((tert-Butyldimethylsilyl)oxy)methyl)piperidin-1-yl)pyridin-3-yl)-4-(3-chloro-4-cyanophenyl)-2,5-dimethylpiperazine-1-carboxamide (D207)

2-Chloro-4-((2R,5S)-2,5-dimethylpiperazin-1-yl)benzonitrile (may be prepared as described Description 143; 266 mg, 1.065 mmol) and phenyl (6-(4-(((tert-butyldimethylsilyl)oxy)methyl)piperidin-1-yl)pyridin-3-yl)carbamate (may be prepared as described in Description 154; 461 mg, 1.044 mmol) were dissolved in acetonitrile (10 mL) and stirred at 70° C. for 2.5 h. The mixture was further heated at 80° C. for 21.5 h, then was left standing for 16 hours. The solvent was evaporated, and the crude residue was dissolved in DCM and chromatographed (0-100% cyclohexane in ethyl acetate) to give (2S,5R)—N-(6-(4-(((tert-butyldimethylsilyl)oxy)methyl)piperidin-1-yl)pyridin-3-yl)-4-(3-chloro-4-cyanophenyl)-2,5-dimethylpiperazine-1-carboxamide (524 mg, 0.877 mmol, 84% yield). LCMS (high pH A): Rt=1.61 min, MH⁺=595.5

### Description 208

### (2S,5R)-4-(3-Chloro-4-cyanophenyl)-N-(6-(4-(hydroxymethyl)piperidin-1-yl)pyridin-3-yl)-2,5-dimethylpiperazine-1-carboxamide (D208)

(2S,5R)—N-(6-(4-(((tert-Butyldimethylsilyl)oxy)methyl)piperidin-1-yl)pyridin-3-yl)-4-(3-chloro-4-cyanophenyl)-2,5-dimethylpiperazine-1-carboxamide (may be prepared as described in Description 207; 509 mg, 0.852 mmol) and hydrogen chloride in 1,4-dioxane (2.130 mL, 8.52 mmol, 4M) were stirred in DCM (5 mL) at room temperature for 30 minutes. The solvent was evaporated, and the crude residue was partitioned between saturated sodium bicarbonate (20 mL) and ethyl acetate (40 mL). The organic phase was separated and washed with water (10 mL×2) and brine (10 mL×3), then dried and evaporated to give (2S,5R)-4-(3-chloro-4-cyanophenyl)-N-(6-(4-(hydroxymethyl)piperidin-1-yl)pyridin-3-yl)-2,5-dimethylpiperazine-1-carboxamide (353 mg, 0.731 mmol, 86% yield). LCMS (high pH A): Rt=1.01 min, MH⁺=483.3, 485.2

### Description 209

### (2S,5R)-4-(3-Chloro-4-cyanophenyl)-N-(6-(4-formylpiperidin-1-yl)pyridin-3-yl)-2,5-dimethylpiperazine-1-carboxamide (D209)

Dimethyl sulfoxide (0.370 mL, 5.22 mmol) was added to oxalyl dichloride (0.221 mL, 2.61 mmol) and 3 Å molecular sieves in DCM (3 mL) at −78° C. under nitrogen. After 15 minutes a solution of (2S,5R)-4-(3-chloro-4-cyanophenyl)-N-(6-(4-(hydroxymethyl)piperidin-1-yl)pyridin-3-yl)-2,5-dimethylpiperazine-1-carboxamide (may be prepared as described in Description 208; 252 mg, 0.522 mmol) in DCM (6 mL) was added slowly. After 15 minutes, triethylamine (1.091 mL, 7.83 mmol) in DCM (6 mL) was added dropwise. The reaction was stirred for a further 15 minutes and then allowed to slowly warm up to room temperature whilst stirring under nitrogen for 2 hours. The reaction mixture was diluted with water (5 mL), and the organic layer was separated. The aqueous layer was then extracted with dichloromethane (2×5 mL). The combined organic extracts were washed with water (5 mL×2) and brine (5 mL×2), evaporated, dissolved in DCM and chromatographed (0-10% ethanol in ethyl acetate) to give (2S,5R)-4-(3-chloro-4-cyanophenyl)-N-(6-(4-formylpiperidin-1-yl)pyridin-3-yl)-2,5-dimethylpiperazine-1-carboxamide (181 mg, 0.376 mmol, 72.1% yield). LCMS (high pH A): Rt=1.09 min, MH⁺=481.3, 483.2

### Description 210

### 2-Chloro-4-((2S,5R)-4-(2-chloropyrimidine-5-carbonyl)-2,5-dimethylpiperazin-1-yl)benzonitrile (D210)

A mixture of 2-chloro-4-((2S,5R)-2,5-dimethylpiperazin-1-yl)benzonitrile (may be prepared as described in Description 142; 350 mg, 1.401 mmol), 2-chloropyrimidine-5-carboxylic acid (200 mg, 1.262 mmol), diisopropylethylamine (218 mg, 0.294 mL, 1.686 mmol) and HATU (580 mg, 1.525 mmol) in DMF (6 mL) was stirred at room temperature for 2.5 h. The reaction mixture was partitioned between ethyl acetate (15 mL) and saturated sodium bicarbonate solution (15 mL), and the aqueous phase was separated. The organic phase was washed with water (10 mL) and brine (10 mL), dried, evaporated and dissolved in DCM. The solution was chromatographed to give 2-chloro-4-((2S,5R)-4-(2-chloropyrimidine-5-carbonyl)-2,5-dimethylpiperazin-1-yl)benzonitrile (164 mg, 0.420 mmol, 33.3% yield). LCMS (high pH A): Rt=1.09 min, MH⁺=no molecular ion.

### Description 211

### 2-Chloro-4-((2S,5R)-4-(2-(4-(hydroxymethyl)piperidin-1-yl)pyrimidine-5-carbonyl)-2,5-dimethylpiperazin-1-yl)benzonitrile (D211)

A mixture of 2-chloro-4-((2S,5R)-4-(2-chloropyrimidine-5-carbonyl)-2,5-dimethylpiperazin-1-yl)benzonitrile (may be prepared as described in Description 210; 164 mg, 0.420 mmol), piperidin-4-ylmethanol (101 mg, 0.877 mmol) and DIPEA (163 mg, 0.220 mL, 1.261 mmol) was stirred in DMF (4 mL) at room temperature for 0.5 h. The reaction mixture was then heated to 60° C. for 16 h, then partitioned between water (20 mL), ethyl acetate (20 mL) and brine (10 mL). The organic phase was washed with water (15 mL) and brine (15 mL), dried and evaporated to give 2-chloro-4-((2S,5R)-4-(2-(4-(hydroxymethyl)piperidin-1-yl)pyrimidine-5-carbonyl)-2,5-dimethylpiperazin-1-yl)benzonitrile (78 mg, 0.166 mmol, 39.6% yield). LCMS (high pH A): Rt=1.04 min, MH⁺=469.2, 471.2

### Description 212

### 2-Chloro-4-((2S,5R)-4-(2-(4-formylpiperidin-1-yl)pyrimidine-5-carbonyl)-2,5-dimethylpiperazin-1-yl)benzonitrile (D212)

2-Chloro-4-((2S,5R)-4-(2-(4-(hydroxymethyl)piperidin-1-yl)pyrimidine-5-carbonyl)-2,5-dimethylpiperazin-1-yl)benzonitrile (may be prepared as described in Description 211; 75 mg, 0.160 mmol) and Dess-Martin Periodinane (102 mg, 0.240 mmol) were dissolved in DCM (3 mL) at room temperature for 0.5 h. The reaction mixture was partitioned between DCM (10 mL) and sodium bicarbonate (10 mL), filtered and the organic phase was separated. The aqueous phase was washed with DCM (10 mL×2). The combined organic phases were washed with water (15 mL) and brine (15 mL), dried and evaporated to give 2-chloro-4-((2S,5R)-4-(2-(4-formylpiperidin-1-yl)pyrimidine-5-carbonyl)-2,5-dimethylpiperazin-1-yl)benzonitrile (59.3 mg, 0.127 mmol, 79% yield). LCMS (high pH A): Rt=1.13 min, MH⁺=467.2, 469.2

### Description 213

### tert-Butyl 4-(4-bromo-1H-indole-7-carbonyl)piperazine-1-carboxylate (D213)

4-Bromo-1H-indole-7-carboxylic acid (330 mg, 1.375 mmol), tert-butyl piperazine-1-carboxylate (307 mg, 1.650 mmol), HATU (627 mg, 1.650 mmol) and DIPEA (0.480 mL, 2.75 mmol) in DMF (2.5 mL) were stirred for 1 h. the reaction was diluted with ethyl acetate, washed with water and brine, dried (MgSO₄), filtered and evaporated in vacuo to give a crude oil. This material was purified by flash chromatography (silica, 40 g, 0-80% EtOAc in cyclohexane) to give tert-butyl 4-(4-bromo-1H-indole-7-carbonyl)piperazine-1-carboxylate (550 mg, 1.280 mmol, 93% yield). LCMS (high pH A): Rt=1.21 min, MH⁺=408.1

### Description 214

### tert-Butyl 4-(4-bromo-5-fluoro-1H-indazol-1-yl)piperidine-1-carboxylate (D214)

A mixture of 4-bromo-5-fluoro-1H-indazole (1.5 g, 6.98 mmol), tert-butyl 4-((methylsulfonyl)oxy)piperidine-1-carboxylate (2.4 g, 8.59 mmol) and cesium carbonate (3.41 g, 10.46 mmol) in anhydrous DMF (30 mL) was stirred at 60° C. for 5 h. More tert-butyl 4-((methylsulfonyl)oxy)piperidine-1-carboxylate (0.6 g) was added, and the mixture was stirred under nitrogen at 60° C. for 18 h. The reaction was allowed to cool to rt and was diluted with EtOAc (50 mL). The solution was washed sequentially with 5% LiCl (aq) (2×50 mL) and brine (50 mL). The organic layer was passed through a hydrophobic frit and the filtrate evaporated in vacuo. The resulting oil was loaded in DCM (5 mL) and purified on a 120 g silica cartridge using a gradient of 0-60% EtOAc in cyclohexane to give tert-butyl 4-(4-bromo-5-fluoro-1H-indazol-1-yl)piperidine-1-carboxylate (1.47 g, 3.69 mmol, 52.9% yield). LCMS (formic A): Rt=1.41 min, M-tBu⁺=342, 344

### Description 215

### tert-Butyl 4-(4-(2,4-dioxo-3-((2-(trimethylsilyl)ethoxy)methyl)tetrahydropyrimidin-1(2H)-yl)-5-fluoro-1H-indazol-1-yl)piperidine-1-carboxylate (D215)

A mixture of copper(I) iodide (0.105 g, 0.554 mmol), potassium carbonate (1.02 g, 7.38 mmol), 3-((2-(trimethylsilyl)ethoxy)methyl)dihydropyrimidine-2,4(1H,3H)-dione (may be prepared as described in Description D1; 1.13 g, 4.62 mmol) and tert-butyl 4-(4-bromo-5-fluoro-1H-indazol-1-yl)piperidine-1-carboxylate (may be prepared as described in Description 214; 1.47 g, 3.69 mmol) was suspended in anhydrous 1,4-dioxane (20 mL). trans-N,N′-Dimethylcyclohexane-1,2-diamine (0.175 mL, 1.107 mmol) was added, and the vessel was sealed, evacuated and purged with nitrogen (×3). The mixture was stirred at 140° C. for 23 h. The reaction was allowed to cool to rt, the suspension filtered through Celite, and the Celite washed with EtOAc (10 mL). The filtrate was evaporated in vacuo and the residue loaded in DCM (5 mL) and purified on an 80 g silica cartridge using a gradient of 0-80% EtOAc in cyclohexane to tert-butyl 4-(4-(2,4-dioxo-3-((2-give (trimethylsilyl)ethoxy)methyl)tetrahydropyrimidin-1(2H)-yl)-5-fluoro-1H-indazol-1-yl)piperidine-1-carboxylate (961 mg, 1.711 mmol, 46.4% yield). LCMS (formic A): Rt=1.39 min, M-C₆H₁₂⁺=478

### Description 216

### 1-(4-Chlorobenzoyl)pyrimidine-2,4(1H,3H)-dione (D216)

To pyrimidine-2,4(1H,3H)-dione (37 g, 330 mmol) in DMF (500 mL) was added pyridine (56.1 mL, 693 mmol) and 4-chlorobenzoyl chloride (63.5 g, 363 mmol) at room temperature. After 16 hr, the reaction was quenched with 10% sodium bicarbonate solution (1 L) and stirred for 30 min. The reaction was filtered and washed with water (1 L). The organic layer was concentrated to give 1-(4-chlorobenzoyl)pyrimidine-2,4(1H,3H)-dione (85.1 g, 263 mmol, 80% yield). LCMS (formic A): Rt=0.735 min, MH⁺=249.0

### Description 217

### 3-((2-(Trimethylsilyl)ethoxy)methyl)pyrimidine-2,4(1H,3H)-dione (D217)

To 1-(4-chlorobenzoyl)pyrimidine-2,4(1H,3H)-dione (may be prepared as described in Description 216; 85 g, 339 mmol) in DMF (1 L) at 0° C. was added NaH (16.28 g, 407 mmol), followed by SEM-Cl (0.078 L, 441 mmol). After 2 hrs at room temperature, the reaction was quenched with water (1.5 L) and extracted with EtOAc (2×1 L). The combined organic layers were washed with brine (500 mL), separated, dried over anhydrous sodium sulphate and concentrated. The residue was dissolved in methanol (1.5 L), and K₂CO₃ (70.3 g, 509 mmol) was added. After 32 h the reaction was concentrated. Water (1 L) was added and was extracted with EtOAc (2×1 L). The combined organic layers were washed with brine (500 mL). The organic layer was dried over anhydrous sodium sulphate, concentrated, dissolved in DCM (150 mL), injected into 750 g silica column, eluting with pet ether/EtOAc as mobile phase to give 3-((2-(trimethylsilyl)ethoxy)methyl)pyrimidine-2,4(1H,3H)-dione (35 g, 129 mmol, 37.9% yield). LCMS (formic A): Rt=0.847 min, MH⁺=241.2

### Description 218

### 1-(1H-Indol-4-yl)-3-((2-(trimethylsilyl)ethoxy)methyl)pyrimidine-2,4(1H,3H)-dione (D218)

To 4-bromo-1H-indole (20.0 g, 102 mmol) in 1,4-dioxane (500 mL) was added 3-((2-(trimethylsilyl)ethoxy)methyl)pyrimidine-2,4(1H,3H)-dione (may be prepared as described in Description 217; 24.72 g, 102 mmol), (1R,2R)—N₁,N₂-dimethylcyclohexane-1,2-diamine (4.35 g, 30.6 mmol) and K₂CO₃ (28.2 g, 204 mmol). After degassing with nitrogen for 5 minutes, copper(I) iodide (2.91 g, 15.30 mmol) was added, and the mixture was heated to 120° C. and stirred for 16 hr. The reaction was filtered through Celite bed and concentrated. Water (200 mL) was added, and was extracted with EtOAc (2×250 mL). The combined organic layers were washed with brine (100 mL), dried over anhydrous sodium sulphate (10 g), concentrated and purified over silica, eluting with pet ether/EtOAc and pet ether to give 1-(1H-indol-4-yl)-3-((2-(trimethylsilyl)ethoxy)methyl)pyrimidine-2,4(1H,3H)-dione (15 g, 35.9 mmol, 35.2% yield). LCMS (formic A): Rt=1.095 min, MH⁺=356.2

### Description 219

### tert-Butyl 4-(4-(2,4-dioxo-3-((2-(trimethylsilyl)ethoxy)methyl)-3,4-dihydropyrimidin-1(2H)-yl)-1H-indol-1-yl)piperidine-1-carboxylate (D219)

To 1-(1H-indol-4-yl)-3-((2-(trimethylsilyl)ethoxy)methyl)pyrimidine-2,4(1H,3H)-dione (may be prepared as described in Description 218; 5.0 g, 13.99 mmol) in DMF (30 mL) was added tert-butyl 4-((methylsulfonyl)oxy)piperidine-1-carboxylate (5.86 g, 20.98 mmol) and cesium carbonate (11.39 g, 35.0 mmol) at room temperature. The reaction was heated to 120° C. and stirred for 16 h. Water (200 mL) was added, and was extracted with EtOAc (2×250 mL). The combined organic layers were washed with brine (100 mL), dried over anhydrous sodium sulphate (10 g), concentrated and purified over silica, eluting with petroleum ether/EtOAc and petroleum ether to give tert-butyl 4-(4-(2,4-dioxo-3-((2-(trimethylsilyl)ethoxy)methyl)-3,4-dihydropyrimidin-1(2H)-yl)-1H-indol-1-yl)piperidine-1-carboxylate (1.3 g, 2.279 mmol, 16.29% yield). LCMS (formic A): Rt=1.356 min, MH⁺=483.2

### Description 220

### tert-Butyl (3R,4S)-3-fluoro-4-(((trifluoromethyl)sulfonyl)oxy)piperidine-1-carboxylate (D220)

To tert-butyl (3R,4S)-3-fluoro-4-hydroxypiperidine-1-carboxylate (0.977 g, 4.46 mmol) in DCM (19.5 ml) was added pyridine (0.901 ml, 11.14 mmol). The reaction was cooled to −25° C. (MeCN dry ice bath) and trifluoroacetic anhydride (0.944 ml, 6.68 mmol) was added dropwise (exotherm to −15° C.). The mixture was stirred at −20° C. for 30 min and diluted with DCM (40 mL) and sat. aq. NaHCO₃ (40 mL). The layers were separated, and the organics were dried by passing through a hydrophobic frit. The solvent was removed in vacuo, and the crude material was purified on a 40 g Redisep silica column, eluting with 2-16% EtOAc in cyclohexane to give tert-butyl (3R,4S)-3-fluoro-4-(((trifluoromethyl)sulfonyl)oxy)piperidine-1-carboxylate (1.20 g, 3.42 mmol, 77% yield). LCMS (high pH A): Rt=1.24 min, M-tBu⁺=251.9

### Description 221

### tert-Butyl (3R,4R)-4-(4-bromo-1H-indol-1-yl)-3-fluoropiperidine-1-carboxylate (D221)

To 4-bromo-1H-indole (200 μL, 1.595 mmol) in DMF (6.6 mL) at 0° C. was added sodium hydride (0.096 g, 2.392 mmol). The reaction was stirred at 0° C. for 5 min, and was tert-butyl (3R,4S)-3-fluoro-4-(((trifluoromethyl)sulfonyl)oxy)piperidine-1-carboxylate (may be prepared as described in Description 220; 1.17 g, 3.33 mmol) was added over 3 portions. After 5 min the reaction was warmed to room temperature and stirred for a further 1 h. The reaction was diluted with EtOAc (80 mL) and washed with sat. aq. NaHCO₃ (40 mL), 5% aq. LiCl (4×20 mL) and brine (40 mL), then dried by passing through a hydrophobic frit. The solvent was removed in vacuo and the residue purified by Combiflash, eluting with 0-10% EtOAc to yield product contaminated with triflate starting material. The crude material was purified by Combiflash, eluting with 0-15% EtOAc in cyclohexane to give tert-butyl (3R,4R)-4-(4-bromo-1H-indol-1-yl)-3-fluoropiperidine-1-carboxylate (364 mg, 0.916 mmol, 57.5% yield). LCMS (high pH A): Rt=1.40 min, M-tBu⁺=341.1, 343.1

### Description 222

### tert-Butyl (3R,4R)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (D222)

3-((2-(Trimethylsilyl)ethoxy)methyl)dihydropyrimidine-2,4(1H,3H)-dione (may be prepared as described in Description D1; 240 mg, 0.982 mmol), potassium carbonate (255 mg, 1.847 mmol), copper(I) iodide (25.7 mg, 0.135 mmol), trans-N,N′-dimethylcyclohexane-1,2-diamine (0.043 mL, 0.270 mmol) and a solution of tert-butyl (3R,4R)-4-(4-bromo-1H-indol-1-yl)-3-fluoropiperidine-1-carboxylate (may be prepared as described in Description 221; 358 mg, 0.901 mmol) in anhydrous 1,4-dioxane (10 mL) were mixed in a microwave vial, sealed, bubbled with nitrogen and heated at 140 C for 15.5 hr. The reaction was cooled to room temperature. The mixture was filtered through filter paper, and the filtrate was evaporated in vacuo and diluted with DCM (15 mL) and water (15 mL). The layers were separated, and the aqueous was extracted with DCM (15 mL). The organics were combined, dried by passing through a hydrophobic frit, concentrated and purified via 24 g Gold Redisep silica column, eluting with 0-50% EtOAc in cyclohexane to give tert-butyl (3R,4R)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (355 mg, 0.538 mmol, 59.7% yield). LCMS (high pH A): Rt=1.39 min, MNa⁺=583.3

### Description 223

### tert-Butyl (3S,4R)-3-fluoro-4-(((trifluoromethyl)sulfonyl)oxy)piperidine-1-carboxylate (D223)

To tert-butyl (3S,4R)-3-fluoro-4-hydroxypiperidine-1-carboxylate (1 g, 4.56 mmol) in DCM (20 ml) was added pyridine (0.922 ml, 11.40 mmol). The reaction was cooled to −30° C. (MeCN dry ice bath) and trifluoroacetic anhydride (0.966 ml, 6.84 mmol) was added dropwise (exotherm to −20° C.). The mixture was stirred at −20° C. for 30 min and diluted with DCM (50 mL) and sat. aq. NaHCO₃ (50 mL). The layers were separated, and the organics were dried by passing through a hydrophobic frit. The solvent was removed in vacuo, and the crude material was purified on a 40 g Redisep silica column, eluting with 2-16% EtOAc in cyclohexane to give tert-butyl (3S,4R)-3-fluoro-4-(((trifluoromethyl)sulfonyl)oxy)piperidine-1-carboxylate (1.32 g, 3.76 mmol, 82%). LCMS (high pH A): Rt=1.23 min, M-tBu⁺=252.0

### Description 224

### tert-Butyl (3S,4S)-4-(4-bromo-1H-indol-1-yl)-3-fluoropiperidine-1-carboxylate (D224)

To 4-bromo-1H-indole (227 μL, 1.811 mmol) in DMF (7.5 mL) at 0° C. was added sodium hydride (0.109 g, 2.72 mmol). The reaction was stirred at 0° C. for 30 min, and tert-butyl (3S,4R)-3-fluoro-4-(((trifluoromethyl)sulfonyl)oxy)piperidine-1-carboxylate (may be prepared as described in Description 223; 1.285 g, 3.66 mmol) was added. After 5 min the reaction was warmed to room temperature and stirred for a further 1 h. The reaction was diluted with EtOAc (80 mL) and washed with saturated aqueous NaHCO₃ (40 mL), 5% aq. LiCl (4×20 mL) and brine (40 mL), then dried by passing through a hydrophobic frit. The solvent was removed in vacuo and the residue purified by Combiflash, eluting with 0-15% EtOAc to yield product contaminated with triflate starting material. The crude material was purified by Combiflash, eluting with 0-12% EtOAc in cyclohexane to give tert-butyl (3S,4S)-4-(4-bromo-1H-indol-1-yl)-3-fluoropiperidine-1-carboxylate (428 mg, 1.077 mmol, 59.5% yield). LCMS (high pH A): Rt=1.41 min, M-tBu⁺=341.1, 343.0

### Description 225

### tert-Butyl (3S,4S)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (D225)

3-((2-(Trimethylsilyl)ethoxy)methyl)dihydropyrimidine-2,4(1H,3H)-dione (may be prepared as described in Description D1; 365 mg, 1.494 mmol), potassium carbonate (386 mg, 2.79 mmol), copper(I) iodide (39 mg, 0.205 mmol), trans-N,N′-dimethylcyclohexane-1,2-diamine (0.064 mL, 0.409 mmol) and a solution of tert-butyl (3S,4S)-4-(4-bromo-1H-indol-1-yl)-3-fluoropiperidine-1-carboxylate (may be prepared as described in Description 224; 541 mg, 1.362 mmol) in anhydrous 1,4-dioxane (15 mL) were mixed in a microwave vial, sealed, bubbled with nitrogen and heated at 140° C. for 15 hr. The reaction was cooled to room temperature. The mixture was filtered through filter paper, and the filtrate was evaporated in vacuo and diluted with DCM (20 mL) and water (20 mL). The layers were separated, and the aqueous was extracted with DCM (20 mL). The organics were combined, dried by passing through a hydrophobic frit, concentrated and purified via 40 g Redisep silica column, eluting with 0-50% EtOAc in cyclohexane to give tert-butyl (3S,4S)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (501 mg, 0.893 mmol, 65.6% yield). LCMS (high pH A): Rt=1.42 min, MH⁺=561.2

### Description 226

### 2-Chloro-4-((2S,5R)-4-(6-(4-(hydroxymethyl)piperidin-1-yl)pyridazine-3-carbonyl)-2,5-dimethylpiperazin-1-yl)benzonitrile (D226)

A mixture of 6-(4-(hydroxymethyl)piperidin-1-yl)pyridazine-3-carboxylic acid (may be prepared as described in Description 61; 200 mg, 0.843 mmol), diisopropylethylamine (218 mg, 0.294 mL, 1.686 mmol) and HATU (385 mg, 1.012 mmol) in DMF (6 mL) was stirred at room temperature for 3 h. A solution of 2-chloro-4-((2S,5R)-2,5-dimethylpiperazin-1-yl)benzonitrile (may be prepared as described in Description 142; 253 mg, 1.012 mmol) in DMF (3 mL) was added to the reaction mixture. The mixture was stirred for 2 h, then partitioned between ethyl acetate (15 mL) and saturated sodium bicarbonate solution (15 mL). The aqueous phase was separated. The organic phase was washed with water (10 mL) and brine (10 mL), then concentrated. The residue was dissolved in DCM and chromatographed, eluting with 0-10% ethanol in ethyl acetate to give 2-chloro-4-((2S,5R)-4-(6-(4-(hydroxymethyl)piperidin-1-yl)pyridazine-3-carbonyl)-2,5-dimethylpiperazin-1-yl)benzonitrile (93.5 mg, 0.199 mmol, 23.65% yield). LCMS (high pH A): Rt=0.98 min, MH⁺=469, 471

### Description 227

### 2-Chloro-4-((2S,5R)-4-(6-(4-formylpiperidin-1-yl)pyridazine-3-carbonyl)-2,5-dimethylpiperazin-1-yl)benzonitrile (D227)

2-Chloro-4-((2S,5R)-4-(6-(4-(hydroxymethyl)piperidin-1-yl)pyridazine-3-carbonyl)-2,5-dimethylpiperazin-1-yl)benzonitrile (may be prepared as described in Description 226; 79 mg, 0.168 mmol) and Dess-Martin Periodinane (143 mg, 0.337 mmol) in DCM (5 mL) were stirred at room temperature for 0.5 h. The reaction mixture was partitioned between DCM (10 mL) and sodium bicarbonate (10 mL). The mixture was filtered, and the organic phase was separated. The aqueous phase was washed with DCM (10 mL×2). The organic phase was washed with water (30 mL) and brine (30 mL), and the organic phase was dried and evaporated to give 2-chloro-4-((2S,5R)-4-(6-(4-formylpiperidin-1-yl)pyridazine-3-carbonyl)-2,5-dimethylpiperazin-1-yl)benzonitrile (79 mg, 0.169 mmol, 100% yield). LCMS (high pH A): Rt=1.06 min, MH⁺=467.2, 469.2

### Description 228

### 2-Chloro-4-((2S,5R)-4-(5-(4-(hydroxymethyl)piperidin-1-yl)pyrazine-2-carbonyl)-2,5-dimethylpiperazin-1-yl)benzonitrile (D228)

A mixture of 2-chloro-4-((2S,5R)-2,5-dimethylpiperazin-1-yl)benzonitrile (may be prepared as described in Description 142; 284 mg, 1.137 mmol), 5-(4-(hydroxymethyl)piperidin-1-yl)pyrazine-2-carboxylic acid (may be prepared as described in Description 62; 200 mg, 0.843 mmol), diisopropylethylamine (218 mg, 0.294 mL, 1.686 mmol) and HATU (424 mg, 1.115 mmol) in DMF (9 mL) was stirred at room temperature for 5 h. The mixture was partitioned between ethyl acetate (15 mL) and saturated sodium bicarbonate solution (15 mL), and the aqueous phase was separated. The organic phase was washed with water (10 mL) and brine (10 mL), and concentrated. The residue was chromatographed over silica, eluting with 0-10% ethanol in ethyl acetate to give 2-chloro-4-((2S,5R)-4-(5-(4-(hydroxymethyl)piperidin-1-yl)pyrazine-2-carbonyl)-2,5-dimethylpiperazin-1-yl)benzonitrile (258 mg, 0.550 mmol, 65.3% yield). LCMS (high pH A): Rt=1.05 min, MH⁺=469.2, 471.2

### Description 229

### 2-Chloro-4-((2S,5R)-4-(5-(4-formylpiperidin-1-yl)pyrazine-2-carbonyl)-2,5-dimethylpiperazin-1-yl)benzonitrile (D229)

2-Chloro-4-((2S,5R)-4-(5-(4-(hydroxymethyl)piperidin-1-yl)pyrazine-2-carbonyl)-2,5-dimethylpiperazin-1-yl)benzonitrile (may be prepared as described in Description 228; 258 mg, 0.550 mmol) and Dess-Martin Periodinane (474 mg, 1.118 mmol) in DCM (4 mL) were stirred for 0.5 h. The reaction was partitioned between DCM (20 mL) and sodium bicarbonate (10 mL), filtered, and the organic phase was separated. The aqueous phase was extracted with DCM (10 mL×2), and the combined organic phases were washed with water (25 mL) and brine (25 mL), then evaporated to give 2-chloro-4-((2S,5R)-4-(5-(4-formylpiperidin-1-yl)pyrazine-2-carbonyl)-2,5-dimethylpiperazin-1-yl)benzonitrile (373 mg, 0.799 mmol, 145% yield). LCMS (high pH A): Rt=1.13 min, MH⁺=467.2, 469.2

### Description 230

### tert-Butyl 4-(4-bromo-1H-pyrrolo[3,2-c]pyridin-1-yl)piperidine-1-carboxylate (D230)

To 4-bromo-1H-pyrrolo[3,2-c]pyridine (2 g, 10.15 mmol) in DMF (30 mL) was added NaH (0.812 g, 20.30 mmol). After 15 min, one-third of the tert-butyl 4-((methylsulfonyl)oxy)piperidine-1-carboxylate (8.51 g, 30.5 mmol) was added, and the reaction was heated to 80° C. At approximately 1 h intervals, the remaining ⅔ tert-butyl 4-((methylsulfonyl)oxy)piperidine-1-carboxylate was added, and the mixture was stirred for 3 h, then allowed to cool over the weekend (approximately 60 h). The reaction was diluted with NH₄Cl solution and ethyl acetate, washed with brine (×2), dried (MgSO₄), filtered and evaporated in vacuo to give a thick oil. This material was purified over a silica 220 g column, eluting with 0-100% EtOAc in cyclohexane to give partially pure product. This material was repurified over a silica 220 g column, eluting with 0-30% diethyl ether in DCM to give tert-butyl 4-(4-bromo-1H-pyrrolo[3,2-c]pyridin-1-yl)piperidine-1-carboxylate (3.1 g, 7.74 mmol, 76% yield). LCMS (high pH A): Rt=1.21 min, MH⁺=381.9

### Description 231

### tert-Butyl 4-(4-(2,4-dioxo-3-((2-(trimethylsilyl)ethoxy)methyl)tetrahydropyrimidin-1(2H)-yl)-1H-pyrrolo[3,2-c]pyridin-1-yl)piperidine-1-carboxylate (D231)

A mixture of 3-((2-(trimethylsilyl)ethoxy)methyl)dihydropyrimidine-2,4(1H,3H)-dione (may be prepared as described in Description D1; 1.671 g, 6.84 mmol) tert-butyl 4-(4-bromo-1H-pyrrolo[3,2-c]pyridin-1-yl)piperidine-1-carboxylate (may be prepared as described in Description 230; 2 g, 5.26 mmol) potassium carbonate (2.181 g, 15.78 mmol) trans-N,N′-dimethylcyclohexane-1,2-diamine (0.249 mL, 1.578 mmol) and copper(I) iodide (0.150 g, 0.789 mmol) in anhydrous 1,4-dioxane (12 mL) was degassed with vacuum/N₂ several times, then heated at 135° C. in a sealed tube overnight (ca 16 h). the reaction was cooled, diluted with water, extracted with ethyl acetate, washed with brine twice, dried, filtered and evaporated in vacuo to give a pale brown oil which was purified over a silica 120 g column, eluting with 50-100% EtOAc in cyclohexane to give tert-butyl 4-(4-(2,4-dioxo-3-((2-(trimethylsilyl)ethoxy)methyl)tetrahydropyrimidin-1(2H)-yl)-1H-pyrrolo[3,2-c]pyridin-1-yl)piperidine-1-carboxylate (1.8 g, 3.14 mmol, 59.8% yield). LCMS (high pH A): Rt=1.34 min, MH⁺=544.3

### Description 232

### tert-Butyl 4-(4-bromo-1H-pyrrolo[2,3-b]pyridin-1-yl)piperidine-1-carboxylate (D232)

To tert-butyl 4-hydroxypiperidine-1-carboxylate (3.89 g, 19.34 mmol), PPh₃ (3.38 g, 12.89 mmol) and 4-bromo-1H-pyrrolo[2,3-b]pyridine (1.27 g, 6.45 mmol) in THF (30 mL) at 0° C. was added, dropwise over about 10 min, DIAD (2.506 mL, 12.89 mmol), and the reaction was stirred vigorously while warming to room temperature overnight (approximately 16 h). The mixture was evaporated in vacuo and purified over a silica, eluting with 0-30% EtOAc in cyclohexane) to give tert-butyl 4-(4-bromo-1H-pyrrolo[2,3-b]pyridin-1-yl)piperidine-1-carboxylate (1.3 g, 3.25 mmol, 50.4% yield). LCMS (high pH A): Rt=1.39 min, MH⁺=382.0

### Description 233

### tert-Butyl 4-(4-(2,4-dioxo-3-((2-(trimethylsilyl)ethoxy)methyl)tetrahydropyrimidin-1(2H)-yl)-1H-pyrrolo[2,3-b]pyridin-1-yl)piperidine-1-carboxylate (D233)

A mixture of 3-((2-(trimethylsilyl)ethoxy)methyl)dihydropyrimidine-2,4(1H,3H)-dione (may be prepared as described in Description D1; 1.086 g, 4.44 mmol) tert-butyl 4-(4-bromo-1H-pyrrolo[2,3-b]pyridin-1-yl)piperidine-1-carboxylate (may be prepared as described in Description 232; 1.3 g, 3.42 mmol), potassium carbonate (0.945 g, 6.84 mmol), trans-N,N′-dimethylcyclohexane-1,2-diamine (0.162 mL, 1.026 mmol) and copper(I) iodide (0.098 g, 0.513 mmol) in anhydrous 1,4-dioxane (13 mL) was degassed with vacuum/N₂ several times, then heated at 135° C. in a sealed tube overnight (ca 16 h). the reaction was cooled, diluted with water, extracted with ethyl acetate, washed with brine twice, dried, filtered and evaporated in vacuo to give a pale brown oil which was purified over a silica 80 g column, eluting with 50-100% EtOAc in cyclohexane to give tert-butyl 4-(4-(2,4-dioxo-3-((2-(trimethylsilyl)ethoxy)methyl)tetrahydropyrimidin-1(2H)-yl)-1H-pyrrolo[2,3-b]pyridin-1-yl)piperidine-1-carboxylate (1.4 g, 2.446 mmol, 71.6% yield). LCMS (high pH A): Rt=1.36 min, MH⁺=544.4

### Description 234

### tert-Butyl (3S,4S)-3-fluoro-4-(((trifluoromethyl)sulfonyl)oxy)piperidine-1-carboxylate (D234)

To tert-butyl (3S,4S)-3-fluoro-4-hydroxypiperidine-1-carboxylate (0.855 g, 3.90 mmol) in DCM (17 ml) was added pyridine (0.789 ml, 9.75 mmol). The reaction was cooled to −20° C. (MeCN dry ice bath) and trifluoromethanesulfonic anhydride (0.826 ml, 5.85 mmol) was added dropwise (exotherm to −10° C.). The mixture was stirred at −10° C. for 30 min and diluted with DCM (40 mL) and sat. aq. NaHCO₃ (40 mL). The layers were separated, and the organics were dried by passing through a hydrophobic frit. The solvent was removed in vacuo, and the crude material was purified on a 40 g Redisep silica column, eluting with 2-18% EtOAc in cyclohexane to give tert-butyl (3S,4S)-3-fluoro-4-(((trifluoromethyl)sulfonyl)oxy)piperidine-1-carboxylate (1.32 g, 3.76 mmol, 96%). LCMS (high pH A): Rt=1.26 min, M-tBu⁺=252.1

### Description 235

### tert-Butyl (3S,4R)-4-(4-bromo-1H-indol-1-yl)-3-fluoropiperidine-1-carboxylate (D235)

To 4-bromo-1H-indole (230 μL, 1.834 mmol) in DMF (7.6 mL) at 0° C. was added sodium hydride (0.110 g, 2.75 mmol). The reaction was stirred at 0° C. for 30 min, and tert-butyl (3S,4S)-3-fluoro-4-(((trifluoromethyl)sulfonyl)oxy)piperidine-1-carboxylate (may be prepared as described in Description 234; 1.26 g, 3.59 mmol) was added. After 5 min the reaction was warmed to room temperature and stirred for a further 1 h. The reaction was diluted with EtOAc (80 mL) and washed with saturated aqueous NaHCO₃ (40 mL). The aqueous was extracted with EtOAc (25 mL). The organics were combined, washed with 5% aq. LiCl (4×20 mL) and brine (40 mL), dried by passing through a hydrophobic frit. The solvent was removed in vacuo and the residue purified by Combiflash, eluting with 0-15% EtOAc to give partially pure product. The crude material was dissolved in minimal 1:1 DMSO:methanol, loaded onto a preconditioned 60 g SNAP C18 column and purified by Biotage Isolera using a gradient of 50-80% acetonitrile in water with an ammonium carbonate modifier to give tert-butyl (3S,4R)-4-(4-bromo-1H-indol-1-yl)-3-fluoropiperidine-1-carboxylate (392 mg, 0.987 mmol, 53.8% yield). LCMS (high pH A): Rt=1.40 min, M-tBu⁺=341.0, 342.9

### Description 236

### tert-Butyl (3S,4R)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (D236)

3-((2-(Trimethylsilyl)ethoxy)methyl)dihydropyrimidine-2,4(1H,3H)-dione (may be prepared as described in Description D1; 263 mg, 1.075 mmol), potassium carbonate (280 mg, 2.0.23 mmol), copper(I) iodide (28.2 mg, 0.148 mmol), trans-N,N′-dimethylcyclohexane-1,2-diamine (0.047 mL, 0.296 mmol) and a solution of tert-butyl (3S,4R)-4-(4-bromo-1H-indol-1-yl)-3-fluoropiperidine-1-carboxylate (may be prepared as described in Description 235; 392 mg, 0.987 mmol) in anhydrous 1,4-dioxane (11.3 mL) were mixed in a microwave vial, sealed, bubbled with nitrogen and heated at 140 C for 21 h. The reaction was cooled to room temperature. The mixture was filtered through filter paper, and the filtrate was evaporated in vacuo and diluted with DCM (15 mL) and water (15 mL). The layers were separated, and the aqueous was extracted with DCM (15 mL). The organics were combined, dried by passing through a hydrophobic frit, concentrated and purified via 40 g Redisep silica column, eluting with 0-50% EtOAc in cyclohexane to give tert-butyl (3S,4R)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (416 mg, 0.742 mmol, 75% yield). LCMS (high pH A): Rt=1.38 min, MH⁺=578.2

### Description 237

### tert-Butyl (3R,4R)-3-fluoro-4-(((trifluoromethyl)sulfonyl)oxy)piperidine-1-carboxylate (D237)

To tert-butyl (3R,4R)-3-fluoro-4-hydroxypiperidine-1-carboxylate (0.989 g, 4.51 mmol) in DCM (19.5 ml) was added pyridine (0.912 ml, 11.28 mmol). The reaction was cooled to −25° C. (MeCN dry ice bath) and trifluoroacetic anhydride (0.956 ml, 6.77 mmol) was added dropwise (exotherm to −15° C.). The mixture was stirred at −20° C. for 30 min and diluted with DCM (40 mL) and saturated aqueous NaHCO₃ (40 mL). The layers were separated, and the organics were dried by passing through a hydrophobic frit. The solvent was removed in vacuo, and the crude material was purified on a 40 g Redisep silica column, eluting with 2-16% EtOAc in cyclohexane to give tert-butyl (3R,4R)-3-fluoro-4-(((trifluoromethyl)sulfonyl)oxy)piperidine-1-carboxylate (1.50 g, 4.27 mmol, 95% yield). LCMS (high pH A): Rt=1.27 min, M-tBu⁺=251.9

### Description 238

### tert-Butyl (3R,4S)-4-(4-bromo-1H-indol-1-yl)-3-fluoropiperidine-1-carboxylate (D238)

To 4-bromo-1H-indole (250 μL, 1.993 mmol) in DMF (8.5 mL) at 0° C. was added sodium hydride (0.120 g, 2.99 mmol). The reaction was stirred at 0° C. for 5 min, and tert-butyl (3R,4R)-3-fluoro-4-(((trifluoromethyl)sulfonyl)oxy)piperidine-1-carboxylate (may be prepared as described in Description 237; 1.44 g, 4.10 mmol) was added. After 5 min the reaction was warmed to room temperature and stirred for a further 1 h. The reaction was diluted with EtOAc (80 mL) and washed with saturated aqueous NaHCO₃ (40 mL), 5% aqueous LiCl (4×20 mL) and brine (40 mL). The organic layer was dried by passing through a hydrophobic frit. The solvent was removed in vacuo and the residue was dissolved in minimal 1:1 DMSO:methanol, loaded onto a preconditioned 60 g SNAP C18 column and purified by Biotage Isolera using a gradient of 50-75% acetonitrile in water with an ammonium carbonate modifier to give tert-butyl (3R,4S)-4-(4-bromo-1H-indol-1-yl)-3-fluoropiperidine-1-carboxylate (429 mg, 1.080 mmol, 54.2% yield). LCMS (high pH A): Rt=1.40 min, MH⁺=397.1, 399.0

### Description 239

### tert-Butyl (3R,4S)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (D239)

3-((2-(Trimethylsilyl)ethoxy)methyl)dihydropyrimidine-2,4(1H,3H)-dione (may be prepared as described in Description D1; 288 mg, 1.177 mmol), potassium carbonate (306 mg, 2.214 mmol), copper(I) iodide (30.8 mg, 0.162 mmol), trans-N,N′-dimethylcyclohexane-1,2-diamine (0.051 mL, 0.324 mmol) and a solution of tert-butyl (3R,4S)-4-(4-bromo-1H-indol-1-yl)-3-fluoropiperidine-1-carboxylate (may be prepared as described in Description 238; 429 mg, 1.080 mmol) in anhydrous 1,4-dioxane (12.4 mL) were mixed in a microwave vial, sealed, bubbled with nitrogen and heated at 140 C for 21 hr. The reaction was cooled to room temperature. The mixture was filtered through filter paper, and the filtrate was evaporated in vacuo and diluted with DCM (15 mL) and water (15 mL). The layers were separated, and the aqueous was extracted with DCM (15 mL). The organics were combined, dried by passing through a hydrophobic frit, concentrated and purified via 40 g Redisep silica column, eluting with 0-50% EtOAc in cyclohexane to give tert-butyl (3R,4S)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (409 mg, 0.729 mmol, 67.5% yield). LCMS (high pH A): Rt=1.38 min, M-tBu⁺=505.3

### Description 240

### tert-Butyl ((1r,4r)-4-((3-chloro-4-cyanophenyl)(methyl)amino)cyclohexyl)carbamate (D240)

A mixture of 2-chloro-4-fluorobenzonitrile (600 mg, 3.86 mmol), tert-butyl ((1r,4r)-4-(methylamino)cyclohexyl)carbamate (969 mg, 4.24 mmol) and DIPEA (1.347 ml, 7.71 mmol) in DMSO (5 ml) was heated at 70° C. for 24 h. The mixture was diluted with water (30 ml) and extracted with EtOAc (2×30 ml). The combined organics were washed with water, dried and evaporated in vacuo to give a yellow gummy solid. This was triturated with ether (30 ml), stirring for 20 minutes, then the solid collected by filtration to give tert-butyl ((1r,4r)-4-((3-chloro-4-cyanophenyl)(methyl)amino)cyclohexyl)carbamate (0.97 g, 2.67 mmol, 69.1% yield). LCMS (high pH A): Rt=1.32 min, MH⁺=364.0, 365.9

### Description 241

### 4-(((1r,4r)-4-Aminocyclohexyl)(methyl)amino)-2-chlorobenzonitrile (D241)

TFA (3 mL, 38.9 mmol) was added to a solution of tert-butyl ((1r,4r)-4-((3-chloro-4-cyanophenyl)(methyl)amino)cyclohexyl)carbamate (may be prepared as described in Description 240; 0.97 g, 2.67 mmol) in DCM (20 mL). The reaction was stirred for 2 h at room temp, then evaporated in vacuo. The residue was stirred in water (20 ml), basified with solid potassium carbonate and extracted with DCM (3×10 ml). The organics were dried and evaporated in vacuo to give 4-(((1r,4r)-4-aminocyclohexyl)(methyl)amino)-2-chlorobenzonitrile (605 mg, 2.294 mmol, 86% yield). LCMS (high pH A): Rt=0.96 min, MH⁺=264.0, 265.9

### Description 242

### 6-Chloropyridazine-3-carbonyl chloride (D242)

DMF (0.05 mL, 0.646 mmol) was added to a suspension of 6-chloropyridazine-3-carboxylic acid (1.6 g, 10.09 mmol) and oxalyl chloride (2 mL, 22.85 mmol) in DCM (20 mL), and the mixture was stirred for 1 h. The solvent was evaporated in vacuo, and the reaction was redissolved in DCM (20 ml) and evaporated to dryness to give 6-chloropyridazine-3-carbonyl chloride (1.83 g, 10.34 mmol, 102% yield). LCMS (high pH A): Rt=0.53 min, M-Cl+MeO⁺=172.9, 174.9

### Description 243

### 6-Chloro-N-((1r,4r)-4-((3-chloro-4-cyanophenyl)(methyl)amino)cyclohexyl)pyridazine-3-carboxamide (D243)

4-(((1r,4r)-4-Aminocyclohexyl)(methyl)amino)-2-chlorobenzonitrile (may be prepared as described in Description 241; 300 mg, 1.137 mmol) and DIPEA (0.596 mL, 3.41 mmol) were dissolved in DCM (10 ml) and cooled in an ice bath. 6-Chloropyridazine-3-carbonyl chloride (may be prepared as described in Description 242; 242 mg, 1.365 mmol) was added, and the mixture was stirred for 2 h, allowing it to warm to room temperature. The solution was washed with water, dried and evaporated in vacuo to give a grey solid. The crude was triturated with EtOAc (10 ml), then filtered to give 6-chloro-N-((1r,4r)-4-((3-chloro-4-cyanophenyl)(methyl)amino)cyclohexyl)pyridazine-3-carboxamide (280 mg, 0.693 mmol, 60.9% yield). LCMS (high pH A): Rt=1.19 min, MH⁺=403.9

### Description 244

### tert-Butyl 4-((4-(4-(2,4-dioxotetrahydropyrimidin-1(2H)-yl)-1H-indol-1-yl)piperidin-1-yl)methyl)piperidine-1-carboxylate (D244)

A suspension of 1-(1-(piperidin-4-yl)-1H-indol-4-yl)dihydropyrimidine-2,4(1H,3H)-dione (may be prepared as described in Example ECB1; 425 mg, 1.361 mmol) and tert-butyl 4-formylpiperidine-1-carboxylate (435 mg, 2.041 mmol) in DCM (8 mL) was treated with sodium triacetoxyborohydride (721 mg, 3.40 mmol), and the mixture was stirred in a stoppered vessel at room temperature for 5 h. The reaction mixture was diluted with DCM (10 mL) and washed sequentially with saturated NaHCO₃ (aqueous) (2×15 mL) and brine (15 mL). The organic layer was passed through a hydrophobic frit, and the filtrate was evaporated in vacuo to give tert-butyl 4-((4-(4-(2,4-dioxotetrahydropyrimidin-1(2H)-yl)-1H-indol-1-yl)piperidin-1-yl)methyl)piperidine-1-carboxylate (776 mg, 1.523 mmol). LCMS (high pH A): Rt=1.21 min, MH⁺=510

### Description 245

### tert-Butyl (R)-3-((4-bromo-1H-indol-1-yl)methyl)-3-fluoropiperidine-1-carboxylate (D245)

Triflic anhydride (5.50 mL, 5.50 mmol) was added to a solution of tert-butyl(S)-3-fluoro-3-(hydroxymethyl)piperidine-1-carboxylate (1.07 g, 4.59 mmol) and DIPEA (0.961 mL, 5.50 mmol) in DCM (30 mL) at 0° C. After 20 minutes, the reaction was washed with water, dried and evaporated in vacuo to give a pale yellow oil. This material was dissolved in DMF and added to a suspension of 4-bromoindole (0.65 g, 3.32 mmol) and Cs₂CO₃ (2.242 g, 6.88 mmol) in DMF (10 ml), and the mixture was stirred overnight at room temperature, quenched with water (30 ml) and extracted with EtOAc (2×30 ml). The combined organics were washed with water, dried, evaporated and purified over a 40 g silica column, eluting with 0-40% EtOAc in cyclohexane to give tert-butyl (R)-3-((4-bromo-1H-indol-1-yl)methyl)-3-fluoropiperidine-1-carboxylate (1.04 g, 2.53 mmol, 55.1% yield). LCMS (high pH A): Rt=1.44 min, MH⁺=413.1

### Description 246

### tert-Butyl (R)-3-((4-(2,4-dioxo-3-((2-(trimethylsilyl)ethoxy)methyl)tetrahydropyrimidin-1(2H)-yl)-1H-indol-1-yl)methyl)-3-fluoropiperidine-1-carboxylate (D246)

To BrettPhos Pd G3 (0.198 g, 0.219 mmol), 3-((2-(trimethylsilyl)ethoxy)methyl)dihydropyrimidine-2,4(1H,3H)-dione (may be prepared as described in Description 1; 0.642 g, 2.63 mmol) and potassium phosphate tribasic, (1.161 g, 5.47 mmol) was added a solution of tert-butyl (R)-3-((4-bromo-1H-indol-1-yl)methyl)-3-fluoropiperidine-1-carboxylate (may be prepared as described in Description 245; 0.9 g, 2.188 mmol) in 1,4-dioxane (30 mL). The reaction was degassed (vacuum/nitrogen×3) and then heated under nitrogen to 100° C. for 18 hours (overnight). The mixture was diluted with water (50 ml) and extracted with EtOAc (2×50 ml). The combined organics were washed with brine, dried, evaporated and purified over a 40 g silica column, eluting with 0-100% EtOAc in cyclohexane to give tert-butyl (R)-3-((4-(2,4-dioxo-3-((2-(trimethylsilyl)ethoxy)methyl)tetrahydropyrimidin-1(2H)-yl)-1H-indol-1-yl)methyl)-3-fluoropiperidine-1-carboxylate (0.61 g, 1.061 mmol, 48.5% yield). LCMS (high pH A): Rt=1.42 min, MH⁺=ion not observed

### Description 247

### 2-Chloro-4-((2S,5R)-4-(6-chloronicotinoyl)-2,5-dimethylpiperazin-1-yl)benzonitrile (D247)

A mixture of 2-chloro-4-((2S,5R)-2,5-dimethylpiperazin-1-yl)benzonitrile (may be prepared as described in Description 142; 630 mg, 2.52 mmol), 6-chloronicotinic acid (399 mg, 2.53 mmol), DIPEA (0.881 mL, 5.05 mmol) and HATU (1067 mg, 2.81 mmol) in DMF (5 mL) was stirred at room temperature for 1.5 hours. The mixture was partitioned between ethyl acetate (25 mL) and saturated sodium bicarbonate solution (25 mL). The aqueous phase was separated. The organic phase was washed with water (10 mL) and brine (10 mL), dried, evaporated and chromatographed, eluting with 0-50% cyclohexane in ethyl acetate to give, 2-chloro-4-((2S,5R)-4-(6-chloronicotinoyl)-2,5-dimethylpiperazin-1-yl)benzonitrile (651 mg, 1.672 mmol, 66.3% yield). LCMS (high pH A): Rt=1.12 min, MH⁺=389.1

### Description 248

### 2-Chloro-4-((2S,5R)-4-(6-(4-hydroxybutoxy)nicotinoyl)-2,5-dimethylpiperazin-1-yl)benzonitrile (D248)

Butane-1,4-diol (0.227 mL, 2.57 mmol) and sodium hydride, 60% by weight in mineral oil (104 mg, 2.60 mmol) were stirred in tetrahydrofuran (3 mL) at room temperature for 10 minutes. 2-Chloro-4-((2S,5R)-4-(6-chloronicotinoyl)-2,5-dimethylpiperazin-1-yl)benzonitrile (may be prepared as described in Description 247; 101 mg, 0.259 mmol) was added to the reaction, and the mixture was stirred at 60° C. for 24 h. The reaction was cooled to room temperature and allowed to stand for 24 hours. The mixture was diluted with water (10 mL) and acidified with 5% citric acid solution (pH˜5) and extracted with ethyl acetate (2×10 mL). The combined extracts were dried, evaporated and chromatographed, eluting with 50-100% ethyl acetate in cyclohexane to give 2-chloro-4-((2S,5R)-4-(6-(4-hydroxybutoxy)nicotinoyl)-2,5-dimethylpiperazin-1-yl)benzonitrile (71 mg, 0.160 mmol, 61.8% yield). LCMS (high pH A): Rt=1.04 min, MH⁺=443.0, 445.0

### Description 249

### 2-Chloro-4-((2S,5R)-2,5-dimethyl-4-(6-(4-oxobutoxy)nicotinoyl)piperazin-1-yl)benzonitrile (D249)

2-Chloro-4-((2S,5R)-4-(6-(4-hydroxybutoxy)nicotinoyl)-2,5-dimethylpiperazin-1-yl)benzonitrile (may be prepared as described in Description 248; 76 mg, 0.172 mmol) and Dess-Martin periodinane (191 mg, 1.118 mmol) were dissolved in DCM (3 mL). After 0.5 h, the mixture was partitioned between DCM (10 mL) and sodium bicarbonate (10 mL), filtered and the organic phase was separated. The aqueous phase was extracted with DCM (5 mL×2), and the combined organic phases were washed with water (25 mL) and brine (25 mL), dried and evaporated to give 2-chloro-4-((2S,5R)-2,5-dimethyl-4-(6-(4-oxobutoxy)nicotinoyl)piperazin-1-yl)benzonitrile (66 mg, 0.150 mmol, 87% yield). LCMS (high pH A): Rt=1.11 min, MH⁺=441.0, 443.0

### Description 250

### tert-Butyl 4-(4-bromo-6-(trifluoromethyl)-1H-indazol-1-yl)piperidine-1-carboxylate (D250)

To 4-bromo-6-(trifluoromethyl)-1H-indazole (2 g, 7.55 mmol) in DMF (20 mL) was added NaH (0.604 g, 15.09 mmol). After 15 min, one-third of the tert-butyl 4-((methylsulfonyl)oxy)piperidine-1-carboxylate (6.32 g, 22.64 mmol) was added. After 1 h, another one-third of the mesylate was added, and the reaction was heated at 80° C. The remainder of the mesylate added after about another hour, and the reaction was heated at 80° C. overnight. The reaction was diluted with ethyl acetate and NH₄Cl, washed with brine, dried, filtered, evaporated in vacuo and purified over silica, (120 g), eluting with 0-30% EtOAc in cyclohexane to give, as the first-eluting isomer, tert-butyl 4-(4-bromo-6-(trifluoromethyl)-1H-indazol-1-yl)piperidine-1-carboxylate (1.2 g, 2.54 mmol, 33.7% yield). LCMS (high pH A): Rt=1.56 min, M-tBu⁺=392.0

### Description 251

### tert-Butyl 4-(4-(2,4-dioxo-3-((2-(trimethylsilyl)ethoxy)methyl)tetrahydropyrimidin-1(2H)-yl)-6-(trifluoromethyl)-1H-indazol-1-yl)piperidine-1-carboxylate (D251)

3-((2-(Trimethylsilyl)ethoxy)methyl)dihydropyrimidine-2,4(1H,3H)-dione (may be prepared as described in Description 1; 500 mg, 2.046 mmol), potassium carbonate (435 mg, 3.15 mmol), trans-N,N′-dimethylcyclohexane-1,2-diamine (0.074 mL, 0.472 mmol), copper(I) iodide (45.0 mg, 0.236 mmol), anhydrous 1,4-dioxane (6 mL) and tert-butyl 4-(4-bromo-6-(trifluoromethyl)-1H-indazol-1-yl)piperidine-1-carboxylate (may be prepared as described in Description 250; 706 mg, 1.574 mmol) were mixed, degassed with vacuum/N₂ several times, then heated at 135° C. in a sealed tube overnight (approximately 16 h). the reaction was cooled, diluted with water, extracted with ethyl acetate, washed with brine twice, dried, filtered, evaporated in vacuo and purified over silica (80 g), eluting with 0-50% EtOAc in cyclohexane to give tert-butyl 4-(4-(2,4-dioxo-3-((2-(trimethylsilyl)ethoxy)methyl)tetrahydropyrimidin-1(2H)-yl)-6-(trifluoromethyl)-1H-indazol-1-yl)piperidine-1-carboxylate (600 mg, 0.932 mmol, 59.2% yield). LCMS (high pH A): Rt=1.49 min, M−H⁺=610.3

### Description 252

### 2-Chloro-4-((2S,5R)-4-(6-((5-hydroxypentyl)oxy)nicotinoyl)-2,5-dimethylpiperazin-1-yl)benzonitrile (D252)

Pentane-1,5-diol (817 mg, 7.84 mmol) and sodium hydride, 60% by weight in mineral oil (317 mg, 7.93 mmol) were stirred in tetrahydrofuran (5 mL) at room temperature for 10 minutes. 2-Chloro-4-((2S,5R)-4-(6-chloronicotinoyl)-2,5-dimethylpiperazin-1-yl)benzonitrile (may be prepared as described in Description 247; 300 mg, 0.771 mmol) was added, and the mixture was stirred at 60° C. for 16.5 h. The mixture was diluted with water (10 mL), acidified with 5% citric acid solution (PH˜5) and extracted with ethyl acetate (2×10 mL). The combined extracts were dried, concentrated and chromatographed, eluting with 50-100% ethyl acetate in cyclohexane to give 2-chloro-4-((2S,5R)-4-(6-((5-hydroxypentyl)oxy)nicotinoyl)-2,5-dimethylpiperazin-1-yl)benzonitrile (137 mg, 0.300 mmol, 38.9% yield). LCMS (high pH A): Rt=1.08 min, MH⁺=457.0, 459.0

### Description 253

### 2-Chloro-4-((2S,5R)-2,5-dimethyl-4-(6-((5-oxopentyl)oxy)nicotinoyl)piperazin-1-yl)benzonitrile (D253)

2-Chloro-4-((2S,5R)-4-(6-((5-hydroxypentyl)oxy)nicotinoyl)-2,5-dimethylpiperazin-1-yl)benzonitrile (may be prepared as described in Description 252; 140.5 mg, 0.307 mmol) and Dess-Martin periodinane (197 mg, 0.464 mmol) were stirred in DCM (3 mL). After 1.5 h, the mixture was partitioned between DCM (10 mL) and sodium bicarbonate (10 mL), and the aqueous phase was extracted with DCM (5 mL×2). The combined organic phases were washed with water (25 mL) and brine (25 mL), dried and evaporated to give 2-chloro-4-((2S,5R)-2,5-dimethyl-4-(6-((5-oxopentyl)oxy)nicotinoyl)piperazin-1-yl)benzonitrile (185 mg, 0.407 mmol, 132% yield). LCMS (high pH A): Rt=1.16 min, MH⁺=455.0, 457.0

### Description 254

### (1-(tert-Butoxycarbonyl)piperidin-4-yl) zinc (II) bromide (D254)

Zinc (1.308 g, 20.00 mmol) and lithium chloride (0.848 g, 20.00 mmol) were combined in a flask containing a stirrer bar, and the flask was evacuated and heated with a heat gun for 5 mins, followed by a purge with nitrogen and repeat of this process. Anhydrous THF (9 mL) and 1,2-dibromoethane (0.043 mL, 0.500 mmol) were added, and the grey coloured mixture was heated to 60° C. while stirring for 20 min, followed by cooling to rt. TMS-Cl (0.013 mL, 0.100 mmol) and a solution of iodine (0.013 g, 0.050 mmol) in anhydrous THF (1 mL) were added, and the mixture was heated to 60° C. while stirring for 20 mins followed by cooling to room temperature. A solution of tert-butyl 4-bromopiperidine-1-carboxylate (1.971 mL, 10 mmol) in THF (3 mL) was added dropwise over 2 mins, followed by the addition of a reflux condenser, vacuum/N₂ purges (×3), and heating to 50° C. for 17 h. The resulting mixture was diluted with THF (10 mL) and left to stand for the large solids to settle. The zincate solution was titrated with iodine solution to establish the concentration. I₂ (8.9 mg) in 1 mL THF was decolorized by 0.12 mL of solution (giving 0.29 M zincate) and 17.5 mg of I₂ in 1 mL THF was decolorized by 0.25 mL of solution (giving 0.28 M zincate). The concentration was taken to be 0.28 M, and this solution was used without any further manipulation. No LCMS or NMR could be taken of this unstable material with no chromophore, but the material was successfully used in the next step, indicating its integrity.

### Description 255

### Methyl 6-(1-(tert-butoxycarbonyl)piperidin-4-yl)pyridazine-3-carboxylate (D255)

Methyl 6-chloropyridazine-3-carboxylate (650 mg, 3.77 mmol) and Pd-PEPPSI-IPent (149 mg, 0.188 mmol) were combined in a flask, and the flask was purged with N₂/vacuum (×3). Anhydrous toluene (15 mL) was added, and the mixture was stirred and cooled in an ice water bath for 5 min. (1-(tert-Butoxycarbonyl)piperidin-4-yl) zinc (II) bromide (may be prepared as described in Description 254; 0.28 M in THF, 15.5 mL, 4.34 mmol) was added dropwise over 2 min, followed by warming to rt. After 4.5 h, the reaction was diluted with water (100 mL), EtOAc (100 mL) and HCl (2 M aq, 10 mL), and the layers were separated. The organic layer was filtered through a hydrophobic frit and concentrated. The crude product was dissolved in minimum DCM (4 mL) and purified over a 40 g silica column, eluting with 0-100% EtOAc in cyclohexane to give methyl 6-(1-(tert-butoxycarbonyl)piperidin-4-yl)pyridazine-3-carboxylate (482 mg, 1.500 mmol, 39.8% yield). LCMS (high pH A): Rt=0.94 min, M-BOC⁺=222.0

### Description 256

### 6-(1-(tert-Butoxycarbonyl)piperidin-4-yl)pyridazine-3-carboxylic acid (D256)

To a stirring solution of methyl 6-(1-(tert-butoxycarbonyl)piperidin-4-yl)pyridazine-3-carboxylate (may be prepared as described in Description 255; 413 mg, 1.285 mmol) in dioxane (10 mL) was added sodium hydroxide (2 M aqueous, 3.21 mL, 6.43 mmol). The solution was heated to 60° C. for 4 h followed by cooling to room temperature. The solution was diluted with EtOAc (50 mL) and water (50 ml), and the aqueous layer was acidified to pH 4 with the gradual addition of acetic acid. The layers were separated, and the aqueous layer was extracted with EtOAc (50 mL) and DCM (2×75 mL). The combined organic layers were filtered through a hydrophobic frit and concentrated in vacuo, followed by addition of EtOAc (20 mL), then cyclohexane (50 mL) and concentration in vacuo (to azeotropically remove the residual acetic acid), followed by a repetition of this process. Separately, some product remained in the aqueous layer, so the aqueous layer was acidified to pH 2-3 by the addition of 5% citric acid solution (10 mL) and extracted with EtOAc (2×50 mL). The organic phases were washed with water (20 mL), filtered through a hydrophobic frit and concentrated in vacuo. The two residues were then combined to give 6-(1-(tert-butoxycarbonyl)piperidin-4-yl)pyridazine-3-carboxylic acid (372 mg, 1.210 mmol, 94% yield). LCMS (high pH A): Rt=0.62 min, M-BOC⁺=208.0

### Description 257

### tert-Butyl 4-(6-(((1r,4r)-4-(3-chloro-4-cyanophenoxy)cyclohexyl)carbamoyl)pyridazin-3-yl)piperidine-1-carboxylate (D257)

To a stirring solution of 6-(1-(tert-butoxycarbonyl)piperidin-4-yl)pyridazine-3-carboxylic acid (may be prepared as described in Description 256; 371 mg, 1.207 mmol), 4-(((1r,4r)-4-aminocyclohexyl)oxy)-2-chlorobenzonitrile (may be prepared as described in Description 46; 393 mg, 1.569 mmol) and HATU (551 mg, 1.449 mmol) in DMF (5 mL) was added DIPEA (0.632 mL, 3.62 mmol). After 1 h, the mixture was diluted with EtOAc (50 mL) and water (50 mL), and the layers were separated. The organic layer was washed with water (50 mL), dried (Na₂SO₄), filtered through a hydrophobic frit and concentrated in vacuo. The crude product was dissolved in minimum DCM and purified using a 24 g silica column, eluting with 0-100% EtOAc in cyclohexane to give tert-butyl 4-(6-(((1r,4r)-4-(3-chloro-4-cyanophenoxy)cyclohexyl)carbamoyl)pyridazin-3-yl)piperidine-1-carboxylate (554 mg, 1.026 mmol, 85% yield). LCMS (high pH A): Rt=1.34 min, M-BOC⁺=440.0, 442.0

### Description 258

### N-((1r,4r)-4-(3-Chloro-4-cyanophenoxy)cyclohexyl)-6-(piperidin-4-yl)pyridazine-3-carboxamide (D258)

To tert-butyl 4-(6-(((1r,4r)-4-(3-chloro-4-cyanophenoxy)cyclohexyl)carbamoyl)pyridazin-3-yl)piperidine-1-carboxylate (may be prepared as described in Description 257; 554 mg, 1.026 mmol) in DCM (10 mL) was added TFA (2.371 mL, 30.8 mmol). After 4 h, the solution was concentrated dissolved in EtOAc (30 mL) and washed with saturated aqueous sodium bicarbonate solution (30 mL). The aqueous layer was extracted with EtOAc (30 mL), and the combined organic layers were washed with brine (50 mL), filtered through a hydrophobic frit and concentrated to give N-((1r,4r)-4-(3-chloro-4-cyanophenoxy)cyclohexyl)-6-(piperidin-4-yl)pyridazine-3-carboxamide (462 mg, 1.050 mmol, 102% yield). LCMS (high pH A): Rt=1.08 min, MH⁺=440.0, 442.0

### Description 259

### tert-Butyl 2-(4-(6-(((1r,4r)-4-(3-chloro-4-cyanophenoxy)cyclohexyl)carbamoyl)pyridazin-3-yl)piperidin-1-yl)acetate (D259)

To N-((1r,4r)-4-(3-chloro-4-cyanophenoxy)cyclohexyl)-6-(piperidin-4-yl)pyridazine-3-carboxamide (may be prepared as described in Description 258; 229 mg, 0.521 mmol) and potassium carbonate (180 mg, 1.301 mmol) suspended in DMF (3 mL) was added tert-butyl 2-bromoacetate (0.085 mL, 0.573 mmol). After 1 h, the mixture was diluted with EtOAc (50 mL) and water (50 mL), and the layers were separated. The organic layer was washed with water (50 mL) and brine (25 mL), filtered through a hydrophobic frit and concentrated to give tert-butyl 2-(4-(6-(((1r,4r)-4-(3-chloro-4-cyanophenoxy)cyclohexyl)carbamoyl)pyridazin-3-yl)piperidin-1-yl)acetate (273 mg, 0.493 mmol, 95% yield). LCMS (high pH A): Rt=1.31 min, MH⁺=554.0, 556.0

### Description 260

### 2-(4-(6-(((1r,4r)-4-(3-Chloro-4-cyanophenoxy)cyclohexyl)carbamoyl)pyridazin-3-yl)piperidin-1-yl) acetic acid, trifluoroacetic acid salt (D260)

To tert-butyl 2-(4-(6-(((1r,4r)-4-(3-chloro-4-cyanophenoxy)cyclohexyl)carbamoyl)pyridazin-3-yl)piperidin-1-yl)acetate (may be prepared as described in Description 259; 273 mg, 0.493 mmol) in DCM (5 mL) was added TFA (1.139 mL, 14.78 mmol). After 24 h, the solution was concentrated to give 2-(4-(6-(((1r,4r)-4-(3-chloro-4-cyanophenoxy)cyclohexyl)carbamoyl)pyridazin-3-yl)piperidin-1-yl) acetic acid, trifluoroacetic acid salt (531 mg, 0.868 mmol, 176% yield). LCMS (high pH A): Rt=0.84 min, MH⁺=498.0, 500.0

### Description 261

### tert-Butyl 4-fluoro-4-((((trifluoromethyl)sulfonyl)oxy)methyl)piperidine-1-carboxylate (D261)

To tert-butyl 4-fluoro-4-(hydroxymethyl)piperidine-1-carboxylate (1.0 g, 4.29 mmol) and pyridine (1.695 g, 1.734 mL, 21.43 mmol) in dichloromethane (8 mL) at 0° C. was added a solution of trifluoromethanesulfonic anhydride (1.33 g, 0.797 mL, 4.72 mmol) in dichloromethane (2 mL), dropwise. After 45 minutes. The mixture was diluted with dichloromethane (10 mL) and washed with water (3×5 mL). The organic phase was dried, evaporated and azeotroped with toluene to give tert-butyl 4-fluoro-4-((((trifluoromethyl)sulfonyl)oxy)methyl)piperidine-1-carboxylate (1.566 g, 4.29 mmol, 100% yield), which used without further purification.

### Description 262 tert-Butyl 4-((4-bromo-1H-indol-1-yl)methyl)-4-fluoropiperidine-1-carboxylate (D262)

To 4-bromo-1H-indole (392 mg, 2.00 mmol) in dry DMF (2 mL) at 0° C. was added sodium hydride (60% wt. in oil) (96 mg, 2.4 mmol), portion wise. After 15 minutes, a solution of tert-butyl 4-fluoro-4-((((trifluoromethyl)sulfonyl)oxy)methyl)piperidine-1-carboxylate (may be prepared as described in Description 261; 1.56 g, 4.27 mmol) in dry DMF (3 mL) was added slowly, and the mixture was allowed to warm to room temperature. After 18 hours, the mixture was diluted with water (20 mL) and extracted with diethyl ether (2×20 mL). The combined extracts were washed with water (10 mL) and brine (10 mL), dried, concentrated and chromatographed, eluting with 0-50% ethyl acetate in cyclohexane] to give tert-butyl 4-((4-bromo-1H-indol-1-yl)methyl)-4-fluoropiperidine-1-carboxylate (630 mg, 1.532 mmol, 77% yield). LCMS (high pH A): Rt=1.47 min, M-tBu⁺=354.7

### Description 263

### tert-Butyl 4-((4-(2,4-dioxo-3-((2-(trimethylsilyl)ethoxy)methyl)tetrahydropyrimidin-1(2H)-yl)-1H-indol-1-yl)methyl)-4-fluoropiperidine-1-carboxylate (D263)

A microwave vial was charged with 3-((2-(trimethylsilyl)ethoxy)methyl)dihydropyrimidine-2,4(1H,3H)-dione (may be prepared as described in Description 1; 468 mg, 1.915 mmol), potassium carbonate (423 mg, 3.06 mmol), copper(I) iodide (44 mg, 15 mol %), and (1S,2S)—N₁,N₂-dimethylcyclohexane-1,2-diamine (33 mg, 15 mol %). A solution of tert-butyl 4-((4-bromo-1H-indol-1-yl)methyl)-4-fluoropiperidine-1-carboxylate (may be prepared as described in Description 262; 630 mg, 1.532 mmol) in dry dioxane (10 mL) was added. The vial was flushed with nitrogen, sealed and stirred at 140° C. for 18 hours. The reaction was cooled to room temperature, filtered and concentrated. The residue was partitioned between water (15 mL) and dichloromethane (15 mL), and the organic phase was separated. The aqueous phase was extracted with dichloromethane (10 mL). The combined organics were dried, evaporated and chromatographed, eluting with 0-50% ethyl acetate in cyclohexane to give tert-butyl 4-((4-(2,4-dioxo-3-((2-(trimethylsilyl)ethoxy)methyl)tetrahydropyrimidin-1(2H)-yl)-1H-indol-1-yl)methyl)-4-fluoropiperidine-1-carboxylate (210 mg, 0.365 mmol, 23.85% yield). LCMS (high pH A): Rt=1.43 min, no MH⁺ peak.

### Description 264

### 4-(4-(Hydroxymethyl)piperidin-1-yl)benzoic acid (D264)

A mixture of methyl 4-iodobenzoate (500 mg, 1.908 mmol), piperidin-4-ylmethanol (330 mg, 2.86 mmol), sodium tert-butoxide (367 mg, 3.82 mmol), DavePhos (150 mg, 20 mol %), and tris(dibenzylideneacetone)dipalladium(0) (175 mg, 10 mol %) in dry dioxane (10 mL) was stirred at 100° C. After 4 h, the mixture was cooled to room temperature, filtered through Celite, evaporated and partitioned between ethyl acetate (10 mL) and water (15 mL). The aqueous phase was separated and acidified by the dropwise addition of acetic acid (pH˜5). The aqueous phase was extracted with ethyl acetate (3×10 mL), and the combined extracts were dried and evaporated to give 4-(4-(hydroxymethyl)piperidin-1-yl)benzoic acid (300 mg, 1.275 mmol, 66.8% yield). LCMS (high pH A): Rt=0.38 min, MH⁺=236.0

### Description 265

### N-((1r,4r)-4-(3-Chloro-4-cyanophenoxy)cyclohexyl)-4-(4-(hydroxymethyl)piperidin-1-yl)benzamide (D265)

A mixture of 4-(4-(hydroxymethyl)piperidin-1-yl)benzoic acid (may be prepared as described in Description 264; 300 mg, 1.275 mmol), 4-(((1r,4r)-4-aminocyclohexyl)oxy)-2-chlorobenzonitrile (may be prepared as described in Description 46; 384 mg, 1.530 mmol), diisopropylethylamine (330 mg, 0.445 mL, 2.55 mmol) and HATU (582 mg, 1.530 mmol) in DMF (3 mL) was stirred at room temperature. After 1 hour, saturated sodium bicarbonate solution (15 mL) was added, and the mixture was stirred for 10 minutes. The reaction was extracted with ethyl acetate (3×10 mL). The combined extracts were washed with water (10 mL) and brine (10 mL), dried, evaporated and chromatographed, eluting with 50-100% ethyl acetate in cyclohexane, then 0-30% ethanol in ethyl acetate to give N-((1r,4r)-4-(3-chloro-4-cyanophenoxy)cyclohexyl)-4-(4-(hydroxymethyl)piperidin-1-yl)benzamide (230 mg, 0.491 mmol, 38.5% yield). LCMS (high pH A): Rt=1.11 min, MH⁺=468.0, 470.0

### Description 266

### N-((1r,4r)-4-(3-Chloro-4-cyanophenoxy)cyclohexyl)-4-(4-formylpiperidin-1-yl)benzamide (D266)

Dess-Martin periodinane (95 mg, 0.224 mmol) was added to N-((1r,4r)-4-(3-chloro-4-cyanophenoxy)cyclohexyl)-4-(4-(hydroxymethyl)piperidin-1-yl)benzamide (may be prepared as described in Description 265; 70 mg, 0.150 mmol) in dichloromethane (3 mL). After 2 hours, the mixture was diluted with dichloromethane (5 mL), and saturated sodium bicarbonate solution (5 mL) was added. After 5 minutes, the organic phase was separated. The aqueous phase was extracted with dichloromethane (2×5 mL), and the combined organics were dried and evaporated to give N-((1r,4r)-4-(3-chloro-4-cyanophenoxy)cyclohexyl)-4-(4-formylpiperidin-1-yl)benzamide (69 mg, 0.148 mmol, 99% yield). LCMS (high pH A): Rt=1.19 min, MH⁺=466.0, 468.0

### Description 267

### tert-Butyl 4-(5-fluoro-4-iodo-1H-indol-1-yl)piperidine-1-carboxylate (D267)

5-Fluoro-4-iodo-1H-indole (2.7 g, 10.34 mmol), tert-butyl 4-hydroxypiperidine-1-carboxylate (4.16 g, 20.69 mmol) and (triphenylphosphoranylidene)acetonitrile (6.23 g, 20.69 mmol) in toluene (30 mL) were stirred at 110° C. for 64 h. The reaction was allowed to cool to room temperature, and was evaporated in vacuo. The residue was loaded in DCM (10 mL) and purified on a 120 g silica cartridge using a gradient of 0-60% EtOAc in cyclohexane to give a yellow gum. This material was loaded in DCM (6 mL) and purified on a 55 g aminopropyl (NH₂) cartridge using a gradient of 0-50% EtOAc in cyclohexane to give tert-butyl 4-(5-fluoro-4-iodo-1H-indol-1-yl)piperidine-1-carboxylate (1.16 g, 2.61 mmol, 25.2% yield). LCMS (formic A): Rt=1.50 min, M-tBu⁺=399

### Description 268

### tert-Butyl 4-(4-(2,4-dioxo-3-((2-(trimethylsilyl)ethoxy)methyl)tetrahydropyrimidin-1(2H)-yl)-5-fluoro-1H-indol-1-yl)piperidine-1-carboxylate (D268)

A mixture of copper(I) iodide (0.075 g, 0.392 mmol), potassium carbonate (0.722 g, 5.22 mmol), 3-((2-(trimethylsilyl)ethoxy)methyl)dihydropyrimidine-2,4(1H,3H)-dione (may be prepared as described in Description 1; 0.766 g, 3.13 mmol) and tert-butyl 4-(5-fluoro-4-iodo-1H-indol-1-yl)piperidine-1-carboxylate (may be prepared as described in Description 267; 1.16 g, 2.61 mmol) was suspended in anhydrous 1,4-dioxane (15 mL). trans-N,N′-Dimethylcyclohexane-1,2-diamine (0.124 mL, 0.783 mmol) was added, and the flask was evacuated and purged with nitrogen (×3). The mixture was stirred at 140° C. After 18 h, the reaction was allowed to cool to room temperature, and additional copper(I) iodide (0.075 g, 0.392 mmol), trans-N,N′-dimethylcyclohexane-1,2-diamine (0.124 mL, 0.783 mmol) and 3-((2-(trimethylsilyl)ethoxy)methyl)dihydropyrimidine-2,4(1H,3H)-dione (1.2 g) were added. The flask was evacuated and purged with nitrogen (×3), and the mixture was stirred at 140° C. After 24 h, the reaction was allowed to cool to RT and was diluted with EtOAc (25 mL). The organic was washed sequentially with water (25 mL) and brine (25 mL), and then passed through a hydrophobic frit. The filtrate was evaporated in vacuo and the residue loaded in DCM (5 mL) and purified on an 80 g silica cartridge using a gradient of 0-80% EtOAc in cyclohexane to give tert-butyl 4-(4-(2,4-dioxo-3-((2-(trimethylsilyl)ethoxy)methyl)tetrahydropyrimidin-1(2H)-yl)-5-fluoro-1H-indol-1-yl)piperidine-1-carboxylate (96 mg, 0.171 mmol, 6.56% yield). LCMS (high pH A): Rt=1.45 min, MH⁺=561

### Description 269

### tert-Butyl (R)-3-(4-bromo-1H-indol-1-yl)pyrrolidine-1-carboxylate (D269)

4-Bromo-1H-indole (0.3 mL, 2.392 mmol), tert-butyl(S)-3-hydroxypyrrolidine-1-carboxylate (1.34 g, 7.16 mmol) and 2-(triphenyl-I5-phosphaneylidene)acetonitrile (1.44 g, 4.78 mmol) in toluene (5 mL) were heated at 110° C. for 64 h. The reaction was concentrated in vacuo, dissolved in DCM (1.5 mL) and loaded onto a cyclohexane preconditioned 120 g Redisep silica column, eluting with 0-45% TBME in cyclohexane to give tert-butyl (R)-3-(4-bromo-1H-indol-1-yl)pyrrolidine-1-carboxylate (2 78 mg, 0.761 mmol, 31.8% yield). LCMS (high pH A): Rt=1.44 min, M-tBu⁺=308.9, 310.9

### Description 270

### tert-Butyl (R)-3-(4-(2,4-dioxo-3-((2-(trimethylsilyl)ethoxy)methyl)tetrahydropyrimidin-1(2H)-yl)-1H-indol-1-yl)pyrrolidine-1-carboxylate (D270)

3-((2-(Trimethylsilyl)ethoxy)methyl)dihydropyrimidine-2,4(1H,3H)-dione (may be prepared as described in Description 1; 203 mg, 0.831 mmol), potassium carbonate (215 mg, 1.556 mmol), copper(I) iodide (22 mg, 0.116 mmol), trans-N,N′-dimethylcyclohexane-1,2-diamine (0.036 mL, 0.228 mmol) and a solution of tert-butyl (R)-3-(4-bromo-1H-indol-1-yl)pyrrolidine-1-carboxylate (may be prepared as described in Description 269; 278 mg, 0.761 mmol) in anhydrous 1,4-dioxane (8.8 mL) were mixed in a microwave vial, flushed with nitrogen, sealed and heated at 140° C. After 20 h, the reaction was cooled to room temperature, evaporated in vacuo and diluted with DCM (15 mL) and water (15 mL). The layers were separated, and the aqueous was extracted with DCM (15 mL). The organics were combined, dried by passing through a hydrophobic frit, concentrated, dissolved in minimal DCM and loaded onto a cyclohexane preconditioned 24 g Redisep silica column, eluting 0-50% EtOAc in cyclohexane to give tert-butyl (R)-3-(4-(2,4-dioxo-3-((2-(trimethylsilyl)ethoxy)methyl)tetrahydropyrimidin-1(2H)-yl)-1H-indol-1-yl)pyrrolidine-1-carboxylate (221 mg, 0.418 mmol, 54.9% yield). LCMS (high pH A): Rt=1.38 min, M+Na⁺=551.3

### Description 271

### tert-Butyl(S)-3-(4-bromo-1H-indol-1-yl)pyrrolidine-1-carboxylate (D271)

4-Bromo-1H-indole (0.2 mL, 1.595 mmol), tert-butyl (R)-3-hydroxypyrrolidine-1-carboxylate (910 mg, 4.86 mmol) and 2-(triphenyl-I5-phosphaneylidene)acetonitrile (961 mg, 3.19 mmol) in toluene (3.4 mL) were heated at 110° C. for 68 h. The reaction was concentrated in vacuo, dissolved in minimal DCM and loaded onto a cyclohexane preconditioned 80 g Redisep silica column, eluting with 0-40% TBME in cyclohexane to give tert-butyl(S)-3-(4-bromo-1H-indol-1-yl)pyrrolidine-1-carboxylate (297 mg, 0.813 mmol, 51.0% yield). LCMS (high pH A): Rt=1.44 min, M-tBu⁺=308.9, 310.9

### Description 272

### tert-Butyl(S)-3-(4-(2,4-dioxo-3-((2-(trimethylsilyl)ethoxy)methyl)tetrahydropyrimidin-1(2H)-yl)-1H-indol-1-yl)pyrrolidine-1-carboxylate (D272)

3-((2-(Trimethylsilyl)ethoxy)methyl)dihydropyrimidine-2,4(1H,3H)-dione (may be prepared as described in Description 1; 217 mg, 0.886 mmol), potassium carbonate (230 mg, 1.667 mmol), copper(I) iodide (23.23 mg, 0.122 mmol), trans-N,N′-dimethylcyclohexane-1,2-diamine (0.038 mL, 0.244 mmol) and a solution of tert-butyl(S)-3-(4-bromo-1H-indol-1-yl)pyrrolidine-1-carboxylate (may be prepared as described in Description 271; 297 mg, 0.813 mmol) in anhydrous 1,4-dioxane (9.5 mL) were mixed in a microwave vial, flushed with nitrogen, sealed and heated at 140° C. After 17 hr, the reaction was cooled to room temperature, evaporated in vacuo and diluted with DCM (15 mL) and water (15 mL). The layers were separated, and the aqueous was extracted with DCM (15 mL). The organics were combined, dried by passing through a hydrophobic frit, concentrated, dissolved in minimal DCM and loaded onto a cyclohexane preconditioned 24 g Redisep silica column, eluting 0-50% EtOAc in cyclohexane to give tert-butyl(S)-3-(4-(2,4-dioxo-3-((2-(trimethylsilyl)ethoxy)methyl)tetrahydropyrimidin-1(2H)-yl)-1H-indol-1-yl)pyrrolidine-1-carboxylate (243 mg, 0.460 mmol, 56.5% yield). LCMS (high pH A): Rt=1.38 min, MNH₄⁺=546.3

### Description 273

### tert-Butyl 6-(4-bromo-1H-indol-1-yl)-2-azaspiro[3.3]heptane-2-carboxylate (D273)

4-Bromo-1H-indole (0.3 mL, 2.392 mmol), tert-butyl 6-hydroxy-2-azaspiro[3.3]heptane-2-carboxylate (1530 mg, 7.18 mmol) and 2-(triphenyl-I5-phosphaneylidene)acetonitrile (961 mg, 3.19 mmol) in toluene (1441 mg, 4.78 mmol) were heated at 110° C. for 67 h. The reaction was concentrated in vacuo, dissolved in minimal DCM and loaded onto a cyclohexane preconditioned 110 g SNAP amino D silica column, eluting with 0-30% EtOAc in cyclohexane to give tert-butyl 6-(4-bromo-1H-indol-1-yl)-2-azaspiro[3.3]heptane-2-carboxylate (642 mg, 1.641 mmol, 68.6% yield). LCMS (high pH A): Rt=1.47 min, MH⁺=391.1, 393.1

### Description 274

### tert-Butyl 6-(4-(2,4-dioxo-3-((2-(trimethylsilyl)ethoxy)methyl)tetrahydropyrimidin-1(2H)-yl)-1H-indol-1-yl)-2-azaspiro[3.3]heptane-2-carboxylate (D274)

3-((2-(Trimethylsilyl)ethoxy)methyl)dihydropyrimidine-2,4(1H,3H)-dione (may be prepared as described in Description 1; 274 mg, 1.120 mmol), potassium carbonate (291 mg, 2.106 mmol), copper(I) iodide (29.3 mg, 0.154 mmol), trans-N,N′-dimethylcyclohexane-1,2-diamine (0.049 ml, 0.308 mmol) and a solution of tert-butyl 6-(4-bromo-1H-indol-1-yl)-2-azaspiro[3.3]heptane-2-carboxylate (may be prepared as described in Description 273; 402 mg, 1.027 mmol) in anhydrous 1,4-dioxane (11.5 mL) were mixed in a microwave vial, flushed with nitrogen, sealed and heated at 140° C. After 18 h, the reaction was cooled to room temperature, evaporated in vacuo and diluted with DCM (15 mL) and water (15 mL). The layers were separated, and the aqueous was extracted with DCM (15 mL). The organics were combined, dried by passing through a hydrophobic frit, concentrated, dissolved in minimal DCM and loaded onto a cyclohexane preconditioned 24 g Redisep silica column, eluting 0-50% EtOAc in cyclohexane to give tert-butyl 6-(4-(2,4-dioxo-3-((2-(trimethylsilyl)ethoxy)methyl)tetrahydropyrimidin-1(2H)-yl)-1H-indol-1-yl)-2-azaspiro[3.3]heptane-2-carboxylate (422 mg, 0.761 mmol, 74.0% yield). LCMS (high pH A): Rt=1.43 min, MNH₄⁺=572.2

### Description 275

### 2-Chloro-4-((2S,5R)-4-(6-(2,2-diethoxyethoxy)nicotinoyl)-2,5-dimethylpiperazin-1-yl)benzonitrile (D275)

2,2-Diethoxyethan-1-ol (172 mg, 1.282 mmol) and sodium hydride, 60% by wt. in mineral oil (51 mg, 1.275 mmol) were stirred for 20 minutes. A solution of 2-chloro-4-((2S,5R)-4-(6-chloronicotinoyl)-2,5-dimethylpiperazin-1-yl)benzonitrile (may be prepared as described in Description 247; 162 mg, 0.416 mmol) in tetrahydrofuran (8 mL) was added, and the mixture was stirred at 60° C. for 16.5 h. The reaction was partitioned between ethyl acetate (10 mL) and water (10 mL), and the aqueous phase was extracted with ethyl acetate (10 mL×1). The combined organic phases were washed with water (25 mL) and brine (25 mL), dried, evaporated and chromatographed, eluting with 0-100% ethyl acetate in cyclohexane to give 2-chloro-4-((2S,5R)-4-(6-(2,2-diethoxyethoxy)nicotinoyl)-2,5-dimethylpiperazin-1-yl)benzonitrile (146 mg, 0.300 mmol, 72.0% yield). LCMS (high pH A): Rt=1.26 min, MH⁺=441.0, 443.0

### Description 276

### 2-Chloro-4-((2S,5R)-2,5-dimethyl-4-(6-(2-oxoethoxy)nicotinoyl)piperazin-1-yl)benzonitrile (D276)

2M Hydrochloric acid (3 mL, 6.00 mmol) was added to 2-chloro-4-((2S,5R)-4-(6-(2,2-diethoxyethoxy)nicotinoyl)-2,5-dimethylpiperazin-1-yl)benzonitrile (may be prepared as described in Description 275; 100 mg, 0.205 mmol) in tetrahydrofuran (3 mL). After 3 h, the reaction was heated to 60° C. After 1.5 h, the mixture was partitioned between saturated sodium bicarbonate (10 mL) and ethyl acetate (10 mL), and the aqueous phase was extracted with ethyl acetate (10 mL). The combined organic phases were washed with water (25 mL) and brine (25 mL), dried and evaporated to give 2-chloro-4-((2S,5R)-2,5-dimethyl-4-(6-(2-oxoethoxy)nicotinoyl)piperazin-1-yl)benzonitrile (40 mg, 0.097 mmol, 47.2% yield). LCMS (formic A): Rt=0.89 min, MH⁺=413.1, 415.1

### Description 277

### Benzyl 4-(2,4-dichloro-7H-pyrrolo[2,3-d]pyrimidin-7-yl)piperidine-1-carboxylate (D277)

2,4-Dichloro-7H-pyrrolo[2,3-d]pyrimidine (3 g, 15.96 mmol), triphenylphosphine (8.37 g, 31.9 mmol) and benzyl 4-hydroxypiperidine-1-carboxylate (11.26 g, 47.9 mmol) in THF (75 mL) cooled to 0 C were treated dropwise with diisopropyl azodicarboxylate (6.20 mL, 31.9 mmol) in 10 ml THF. The reaction was allowed to warm to room temperature. After 3 h, the mixture was concentrated in vacuo and purified over silica, 330 g column, eluting with 0-40% EtOAc in cyclohexane to give benzyl 4-(2,4-dichloro-7H-pyrrolo[2,3-d]pyrimidin-7-yl)piperidine-1-carboxylate (6 g, 14.06 mmol, 88% yield). LCMS (high pH A): Rt=1.38 min, MH⁺=405.1

### Description 278

### Benzyl 4-(2-chloro-4-(2,4-dioxo-3-((2-(trimethylsilyl)ethoxy)methyl)tetrahydropyrimidin-1(2H)-yl)-7H-pyrrolo[2,3-d]pyrimidin-7-yl)piperidine-1-carboxylate (D278)

3-((2-(Trimethylsilyl)ethoxy)methyl)dihydropyrimidine-2,4(1H,3H)-dione, (may be prepared as described in Description 1; 1.013 g, 4.15 mmol), benzyl 4-(2,4-dichloro-7H-pyrrolo[2,3-d]pyrimidin-7-yl)piperidine-1-carboxylate (may be prepared as described in Description 277; 1.4 g, 3.45 mmol), anhydrous 1,4-dioxane (14 mL) Xantphos (0.400 g, 0.691 mmol), Pd₂(dba)₃ (0.316 g, 0.345 mmol) and Cs₂CO₃ (2.251 g, 6.91 mmol) were mixed, degassed with vacuum/N₂ several times and heated at 110° C. in a microwave tube for 1 h. The reaction was filtered, and the filtrate was diluted with ethyl acetate and water. The organic layer was washed with brine, dried (MgSO₄), filtered, concentrated in vacuo and purified over silica, 80 g, eluting with 0-40% EtOAc in cyclohexane) to give benzyl 4-(2-chloro-4-(2,4-dioxo-3-((2-(trimethylsilyl)ethoxy)methyl)tetrahydropyrimidin-1(2H)-yl)-7H-pyrrolo[2,3-d]pyrimidin-7-yl)piperidine-1-carboxylate (1.2 g, 1.859 mmol, 53.8% yield). LCMS (high pH A): Rt=1.49 min, MH⁺=613.1, 615.1

### Description 279

### 1-(2-Chloro-7-(piperidin-4-yl)-7H-pyrrolo[2,3-d]pyrimidin-4-yl)-3-((2-(trimethylsilyl)ethoxy)methyl)dihydropyrimidine-2,4(1H,3H)-dione (D279)

Benzyl 4-(2-chloro-4-(2,4-dioxo-3-((2-(trimethylsilyl)ethoxy)methyl)tetrahydropyrimidin-1(2H)-yl)-7H-pyrrolo[2,3-d]pyrimidin-7-yl)piperidine-1-carboxylate (may be prepared as described in Description 278; 1150 mg, 1.875 mmol) and platinum (IV) oxide (85 mg, 0.375 mmol) in isopropanol (12 mL) and HCl (1.0 M aq, 2 mL, 2.000 mmol) hydrogenated at burette pressure. After 2 h, the mixture was filtered; concentrated; taken up in DCM; washed with water, bicarb and brine; dried (MgSO₄); filtered; concentrated and purified over C18 silica, 100 g, eluting with 30-80% MeCN in 10 mM pH10 NH₄CO₂H (aq) to give 1-(2-chloro-7-(piperidin-4-yl)-7H-pyrrolo[2,3-d]pyrimidin-4-yl)-3-((2-(trimethylsilyl)ethoxy)methyl)dihydropyrimidine-2,4(1H,3H)-dione (400 mg, 0.793 mmol, 42.3% yield). LCMS (high pH A): Rt=1.24 min, MH⁺=479.1, 481.1

### Description 280

### 2-Methyl-1,3-dinitro-5-(trifluoromethyl)benzene (D280)

To concentrated sulfuric acid (250 ml, 4690 mmol) in a 500 ml 3-neck round bottomed flask immersed in a water bath, behind a blast shield, was added nitric acid (29.9 ml, 468 mmol), slowly in portions, keeping the temperature below 50° C. 1-Methyl-4-(trifluoromethyl)benzene (30 g, 187 mmol) was added. After 40 h, the mixture was carefully poured into 0.5 kg of ice, extracted with ethyl acetate, washed with brine, dried (MgSO₄), filtered and concentrated. The residue was redissolved in ethyl acetate and washed with bicarb solution, whereupon the mixture turned bright red, both aqueous and organic layers. The aqueous layer was isolated and extracted with ethyl acetate until most of the orange colour had been extracted, then the combined organic layers were washed with brine (2×), dried (MgSO₄), filtered and evaporated in vacuo to give 2-methyl-1,3-dinitro-5-(trifluoromethyl)benzene (44 g, 0.176 mol, 89% yield). LCMS (formic A): Rt=1.15 min, MH⁺=250.1

### Description 281

### 6-(Trifluoromethyl)-1H-indol-4-amine (D281)

To 2-methyl-1,3-dinitro-5-(trifluoromethyl)benzene (may be prepared as described in Description 280; 40 g, 160 mmol) in acetonitrile (160 mL) was added DMF-DMA (64.2 mL, 480 mmol), and the reaction was heated to 80° C. After 0.5 h, the reaction was concentrated in vacuo to give a powder, (E)-2-(2,6-dinitro-4-(trifluoromethyl)phenyl)-N,N-dimethylethen-1-amine, approximately 50 g, which was suspended in acetic acid (400 mL). Separately, iron (73.2 g, 1311 mmol) was stirred vigorously with a mechanical stirrer in a three-neck flask in acetic acid (400 mL) and warmed to 50° C. The heating was turned off and the suspension of (E)-2-(2,6-dinitro-4-(trifluoromethyl)phenyl)-N,N-dimethylethen-1-amine in acetic acid was added in five batches over 1 h, keeping the temperature below 100° C. The temperature was held at 90° C. for 1 h, then the reaction was cooled slightly and filtered through a pad of Celite, washing with acetonitrile. The filtrate was evaporated in vacuo, taken up in ethyl acetate and washed with water/brine, dried (MgSO₄), filtered, concentrated and purified over silica, 330 g, eluting with 0-30% EtOAc in (1:1 DCM/cyclohexane) to give a brown-black solid, which was recrystallized from hot ethyl acetate/cyclohexane to give a 6-(trifluoromethyl)-1H-indol-4-amine (6.5 g, 30.8 mmol, 18.83% yield). LCMS (high pH A): Rt=0.90 min, MH⁺=201.1

### Description 282

### Benzyl 4-(4-amino-6-(trifluoromethyl)-1H-indol-1-yl)piperidine-1-carboxylate (D282)

NaH (0.899 g, 22.48 mmol) and 6-(trifluoromethyl)-1H-indol-4-amine (may be prepared as described in Description 281; 1.5 g, 7.49 mmol) were stirred at room temperature for 15 min, then treated with one-third of the benzyl 4-((methylsulfonyl)oxy)piperidine-1-carboxylate (7.04 g, 22.48 mmol) and heated at 90° C. After 1 h, the reaction was cooled to 0° C. and treated with more NaH (0.899 g, 22.48 mmol), heated to 90° C. and treated with the remaining mesylate in two approximately equal amounts at hourly intervals. The mixture was cooled, diluted with ethyl acetate and NH₄Cl, washed with brine, dried (MgSO₄), filtered, concentrated and purified over silica, 120 g, eluting with 0-40% EtOAc in cyclohexane to give benzyl 4-(4-amino-6-(trifluoromethyl)-1H-indol-1-yl)piperidine-1-carboxylate (1.7 g, 3.71 mmol, 49.5% yield). LCMS (high pH A): Rt=1.30 min, MH⁺=418.2

### Description 283

### Benzyl 4-(4-(2,4-dioxotetrahydropyrimidin-1(2H)-yl)-6-(trifluoromethyl)-1H-indol-1-yl)piperidine-1-carboxylate (D283)

Benzyl 4-(4-amino-6-(trifluoromethyl)-1H-indol-1-yl)piperidine-1-carboxylate (may be prepared as described in Description 282; 1.7 g, 4.07 mmol) and acrylic acid (0.839 mL, 12.22 mmol) were stirred for 24 h, then allowed to stand for 8 h. The reaction was treated with acetic acid (5 mL) and urea (1.223 g, 20.36 mmol) and heated at 130° C. overnight (approximately 16 h). The reaction was concentrated; taken up in ethyl acetate; washed with water, sodium bicarbonate and brine; dried (MgSO₄); filtered; concentrated and purified over silica, 120 g, eluting with 50-100% EtOAc in cyclohexane to give benzyl 4-(4-(2,4-dioxotetrahydropyrimidin-1(2H)-yl)-6-(trifluoromethyl)-1H-indol-1-yl)piperidine-1-carboxylate (1.6 g, 2.95 mmol, 72.5% yield). LCMS (high pH A): Rt=1.19 min, MH⁺=532.1

### Description 284

### (3-Bromopyridin-2-yl) methanamine (D284)

To 3-bromopicolinonitrile (3.25 g, 17.76 mmol) in dry toluene (30 mL) at 0° C. was added alane-N,N-dimethylethylamine complex (0.5M in toluene, 71.0 mL, 35.5 mmol), dropwise over 15 minutes. After complete addition the mixture was allowed to warm to room temperature. After 2 h, the reaction was cooled to 0° C., and methanol (15 mL) was added dropwise (vigorous reaction). A saturated solution of sodium potassium tartrate (Rochelle's salt) (100 mL) was added, and the mixture was stirred at room temperature for 15 minutes. The organic phase was separated, and the aqueous phase was extracted with dichloromethane (2×50 mL). The combined organics were dried and evaporated to give (3-bromopyridin-2-yl) methanamine (2.11 g, 11.28 mmol, 63.5% yield). LCMS (high pH A): Rt=0.58 min, MH⁺=186.9, 188.9

### Description 285

### tert-Butyl 4-(((3-bromopyridin-2-yl)methyl)carbamoyl)piperidine-1-carboxylate (D285)

A mixture of 1-(tert-butoxycarbonyl)piperidine-4-carboxylic acid (2.155 g, 9.4 mmol), (3-bromopyridin-2-yl) methanamine (may be prepared as described in Description 284; 2.11 g, 11.28 mmol), diisopropylethylamine (2.43 g, 3.28 mL, 18.80 mmol) and HATU (4.29 g, 11.28 mmol) in DMF (15 mL) was stirred at room temperature for 2 hours. Saturated sodium bicarbonate solution (50 mL) was added, and the mixture was stirred for 10 minutes. The mixture was extracted with ethyl acetate (3×50 mL). The combined extracts were washed with 5% aqueous citric acid solution (30 mL), water (30 mL) and brine (30 mL). The organic phase was dried, evaporated and chromatographed, eluting with 0-100% ethyl acetate in cyclohexane to give tert-butyl 4-(((3-bromopyridin-2-yl)methyl)carbamoyl)piperidine-1-carboxylate (1.64 g, 4.12 mmol, 43.8% yield). LCMS (high pH A): Rt=1.00 min, MH⁺=399.9

### Description 286

### tert-Butyl 4-(8-bromoimidazo[1,5-a]pyridin-3-yl)piperidine-1-carboxylate (D286)

Phosphorus oxychloride (3.08 g, 1.872 mL, 20.09 mmol) was added slowly to a stirred suspension of tert-butyl 4-(((3-bromopyridin-2-yl)methyl)carbamoyl)piperidine-1-carboxylate (may be prepared as described in Description 285; 1.6 g, 4.02 mmol) in toluene (50 mL), and the reaction was heated to 120° C. After 3 hours, the mixture was cooled to room temperature and basified by the dropwise addition of saturated sodium bicarbonate solution. The mixture was evaporated to dryness, and the resulting product was suspended in tetrahydrofuran (50 mL) and methanol (5 mL). After 15 minutes, the mixture was filtered, and the filtrate was treated with triethylamine (0.406 g, 0.560 mL, 4.02 mmol) and di-tert-butyl dicarbonate (0.877 g, 4.02 mmol). After 2 hours, the solvent was evaporated and the residue was chromatographed, eluting with 10-70% ethyl acetate in cyclohexane to give tert-butyl 4-(8-bromoimidazo[1,5-a]pyridin-3-yl)piperidine-1-carboxylate (425 mg, 1.118 mmol, 27.8% yield). LCMS (formic A): Rt=0.98 min, MH⁺=381.9

### Description 287

### tert-Butyl 4-(8-(2,4-dioxo-3-((2-(trimethylsilyl)ethoxy)methyl)tetrahydropyrimidin-1(2H)-yl) imidazo[1,5-a]pyridin-3-yl)piperidine-1-carboxylate (D287)

tert-Butyl 4-(8-bromoimidazo[1,5-a]pyridin-3-yl)piperidine-1-carboxylate (may be prepared as described in Description 286; 405 mg, 1.657 mmol), potassium carbonate (305 mg, 2.209 mmol), copper(I) iodide (43 mg, 20 mol %), trans-N,N′-dimethylcyclohexane-1,2-diamine (32 mg, 0.035 mL, 20 mol %) and a solution of 3-((2-(trimethylsilyl)ethoxy)methyl)dihydropyrimidine-2,4(1H,3H)-dione (may be prepared as described in Description 1; 185 mg, 0.486 mmol) in anhydrous 1,4-dioxane (10 mL) were mixed in a microwave vial, flushed with nitrogen, sealed and heated at 140° C. After 18 hr, a further portion of copper(I) iodide (43 mg, 20 mol %), and trans-N,N′-dimethylcyclohexane-1,2-diamine (32 mg, 0.035 mL, 20 mol %) was added and the reaction mixture was stirred at 140° C. After 24 h, the reaction was cooled to room temperature, allowed to stand for 5 days, filtered through Celite, evaporated in vacuo and diluted with DCM (15 mL) and water (15 mL). The layers were separated, and the aqueous was extracted with DCM (10 mL). The organics were combined, dried, concentrated, and chromatographed, eluting with 50-100% EtOAc in cyclohexane, then 0-20% EtOH in EtOAc to give tert-butyl 4-(8-(2,4-dioxo-3-((2-(trimethylsilyl)ethoxy)methyl)tetrahydropyrimidin-1(2H)-yl) imidazo[1,5-a]pyridin-3-yl)piperidine-1-carboxylate (287 mg, 0.528 mmol, 47.8% yield). LCMS (high pH A): Rt=1.25 min, MH⁺=544.1

### Description 288

### tert-Butyl 3-((6-(((1r,4r)-4-(3-chloro-4-cyanophenoxy)cyclohexyl)carbamoyl)pyridazin-3-yl)oxy)azetidine-1-carboxylate (D288)

To tert-butyl 3-hydroxyazetidine-1-carboxylate (131 mg, 0.755 mmol) and 60% NaH in mineral oil (32.1 mg, 0.802 mmol) in an ice-water bath was added DMF (3 ml). After 20 mins, 6-chloro-N-((1r,4r)-4-(3-chloro-4-cyanophenoxy)cyclohexyl)pyridazine-3-carboxamide (may be prepared as described in Description 90; 196 mg, 0.501 mmol). After 90 min, whilst warming to room temperature, the mixture was diluted with water (10 ml) and EtOAc (10 ml) and the layers were separated. The organic layer was washed with water (10 ml), passed through a hydrophobic frit and concentrated in vacuo and purified over silica, 24 g column, eluting with 0-100% EtOAc in cyclohexane to give tert-butyl 3-((6-(((1r,4r)-4-(3-chloro-4-cyanophenoxy)cyclohexyl)carbamoyl)pyridazin-3-yl)oxy)azetidine-1-carboxylate (307 mg, 0.581 mmol, 116% yield). LCMS (high pH A): Rt=1.33 min, M-tBu⁺=472.0, 473.9

### Description 289

### 6-(Azetidin-3-yloxy)-N-((1r,4r)-4-(3-chloro-4-cyanophenoxy)cyclohexyl)pyridazine-3-carboxamide (D289)

To tert-butyl 3-((6-(((1r,4r)-4-(3-chloro-4-cyanophenoxy)cyclohexyl)carbamoyl)pyridazin-3-yl)oxy)azetidine-1-carboxylate (may be prepared as described in Description 288; 306 mg, 0.580 mmol) in DCM (5 mL) was added TFA (1.786 mL, 23.18 mmol). After 5 h, the reaction was concentrated, dissolved in DCM (20 mL) and diluted and washed with saturated aqueous sodium hydrogen carbonate solution (20 mL). The aqueous layer was extracted with DCM (20 mL), and the combined organic layers were filtered through a hydrophobic frit and concentrated in vacuo to give 6-(azetidin-3-yloxy)-N-((1r,4r)-4-(3-chloro-4-cyanophenoxy)cyclohexyl)pyridazine-3-carboxamide (210 mg, 0.491 mmol, 85% yield). LCMS (high pH A): Rt=1.01 min, MH⁺=428.1, 430.1

### Description 290

### 1-(1H-Indol-4-yl)dihydropyrimidine-2,4(1H,3H)-dione (D290)

1H-Indol-4-amine (25 g, 189 mmol) and acrylic acid (38.9 mL, 567 mmol) in toluene (100 mL) were stirred at 50° C. After 2 h, the toluene was removed in vacuo and the mixture was treated with AcOH (150 mL) and urea (68.2 g, 1135 mmol) and heated at 140° C. After 18 h, the cooled mixture was treated with water (150 mL) in order to precipitate the product, but this failed (a dark, water immiscible viscous oil persisted). The mixture was evaporated to dryness, and the residue was treated with methanol (60 mL), stirred to form a solution and then treated with water (200 mL). The resulting precipitate was collected by filtration. The filtrate was further concentrated to yield two more crops of precipitate to afford 1-(1H-indol-4-yl)dihydropyrimidine-2,4(1H,3H)-dione (19.2 g, 83.8 mmol, 44.28% yield). LCMS (formic A): Rt=0.52 min, MH⁺=230.1

### Description 291

### 1-(1H-Indol-4-yl)-3-((2-(trimethylsilyl)ethoxy)methyl)dihydropyrimidine-2,4(1H,3H)-dione (D291)

To a suspension of 1-(1H-indol-4-yl)dihydropyrimidine-2,4(1H,3H)-dione (may be prepared as described in Description 290; 2.85 g, 12.43 mmol) in DIPEA (8.6 ml, 49.2 mmol) was added SEM-Cl (6.3 ml, 35.5 mmol) in DCM (8.6 ml). After 20 h, the reaction was diluted with DCM (150 mL) and sat. aq. NaHCO₃ (150 mL). The layers were separated, and the organics were dried by passing through a hydrophobic frit, concentrated and loaded onto a cyclohexane-preconditioned 330 g Redisep silica column, eluting with 0-50% EtOAc in cyclohexane to give 1-(1H-indol-4-yl)-3-((2-(trimethylsilyl)ethoxy)methyl)dihydropyrimidine-2,4(1H,3H)-dione (2.25 g, 6.26 mmol, 50.3% yield). LCMS (high pH A): Rt=1.14 min, MH−=358.3

### Description 292

### tert-Butyl 4-(2-(4-(2,4-dioxo-3-((2-(trimethylsilyl)ethoxy)methyl)tetrahydropyrimidin-1(2H)-yl)-1H-indol-1-yl)ethyl)piperidine-1-carboxylate (D292)

tert-Butyl 4-(2-hydroxyethyl)piperidine-1-carboxylate (2.048 g, 8.93 mmol), 2-(triphenyl-I5-phosphaneylidene)acetonitrile (1.794 g, 5.95 mmol) and 1-(1H-indol-4-yl)-3-((2-(trimethylsilyl)ethoxy)methyl)dihydropyrimidine-2,4(1H,3H)-dione (may be prepared as described in Description 291; 1.070 g, 2.98 mmol) in toluene (6.40 ml) were heated to 110° C. After 18 h, the mixture was allowed to cool to room temperature. The reaction was concentrated, dissolved in minimal DCM and loaded onto a cyclohexane-preconditioned 80 g Redisep silica column, eluting with 0-50% EtOAc in cyclohexane to give tert-butyl 4-(2-(4-(2,4-dioxo-3-((2-(trimethylsilyl)ethoxy)methyl)tetrahydropyrimidin-1(2H)-yl)-1H-indol-1-yl)ethyl)piperidine-1-carboxylate (1.6209 g, 2.84 mmol, 95% yield). LCMS (formic A): Rt=1.49 min, M-BOC⁺=471.3

### Description 293

### tert-Butyl (R)-3-(4-bromo-1H-indol-1-yl)piperidine-1-carboxylate (D293)

4-Bromo-1H-indole (0.422 mL, 3.36 mmol), tert-butyl(S)-3-hydroxypiperidine-1-carboxylate (2031 mg, 10.09 mmol) and 2-(triphenyl-I5-phosphaneylidene)acetonitrile (2534 mg, 8.41 mmol) in toluene (7.16 mL) were heated to 110° C. After 67 h, the reaction was allowed to cool to room temperature, concentrated, dissolved in minimal DCM and loaded onto a cyclohexane-preconditioned 110 g SNAP amino D silica column, and eluting with 0-15% EtOAc in cyclohexane to give tert-butyl (R)-3-(4-bromo-1H-indol-1-yl)piperidine-1-carboxylate (206 mg, 0.407 mmol, 12.11% yield). LCMS (high pH A): Rt=1.51 min, MH⁺=381.1

### Description 294

### tert-Butyl (R)-3-(4-(2,4-dioxo-3-((2-(trimethylsilyl)ethoxy)methyl)tetrahydropyrimidin-1(2H)-yl)-1H-indol-1-yl)piperidine-1-carboxylate (D294)

tert-Butyl (R)-3-(4-bromo-1H-indol-1-yl)piperidine-1-carboxylate (may be prepared as described in Description 293; 238 mg, 0.627 mmol), potassium carbonate (178 mg, 1.286 mmol), copper(I) iodide (18 mg, 0.095 mmol), trans-N,N′-dimethylcyclohexane-1,2-diamine (0.030 mL, 0.19 mmol) and a solution of 3-((2-(trimethylsilyl)ethoxy)methyl)dihydropyrimidine-2,4(1H,3H)-dione (may be prepared as described in Description 1; 167 mg, 0.684 mmol), in anhydrous 1,4-dioxane (7 mL) were mixed in a microwave vial, flushed with nitrogen, sealed and heated at 140° C. After 18 hr, the reaction was cooled to room temperature, allowed to stand for 5 days, filtered through filter paper, evaporated in vacuo and diluted with DCM (10 mL) and water (10 mL). The layers were separated, and the aqueous was extracted with DCM (10 mL). The organics were combined, dried, concentrated, dissolved in minimal DCM and loaded onto a cyclohexane-preconditioned 12 g Redisep silica column, eluting with 0-50% EtOAc in cyclohexane give to tert-butyl (R)-3-(4-(2,4-dioxo-3-((2-(trimethylsilyl)ethoxy)methyl)tetrahydropyrimidin-1(2H)-yl)-1H-indol-1-yl)piperidine-1-carboxylate (168 mg, 0.310 mmol, 49.3% yield). LCMS (high pH A): Rt=1.46 min, MNH₄⁺=560.2

### Description 295

### tert-Butyl(S)-3-(4-bromo-1H-indol-1-yl)piperidine-1-carboxylate (D295)

4-Bromo-1H-indole (0.422 mL, 3.36 mmol), tert-butyl (R)-3-hydroxypiperidine-1-carboxylate (2.03 g, 10.09 mmol) and 2-(triphenyl-I5-phosphaneylidene)acetonitrile (2.53 g, 8.41 mmol) in toluene (7.16 mL) were heated to 110° C. After 67 h, the reaction was allowed to cool to room temperature, concentrated, dissolved in minimal DCM and loaded onto a cyclohexane-preconditioned 110 g SNAP amino D silica column, and eluting with 0-15% EtOAc in cyclohexane to give tert-butyl(S)-3-(4-bromo-1H-indol-1-yl)piperidine-1-carboxylate (265 mg, 0.524 mmol, 15.57% yield). LCMS (high pH A): Rt=1.51 min, M-tBu⁺=323.0, 325.0

### Description 296

### tert-Butyl(S)-3-(4-(2,4-dioxo-3-((2-(trimethylsilyl)ethoxy)methyl)tetrahydropyrimidin-1(2H)-yl)-1H-indol-1-yl)piperidine-1-carboxylate (D296)

tert-Butyl(S)-3-(4-bromo-1H-indol-1-yl)piperidine-1-carboxylate (may be prepared as described in Description 295; 260 mg, 0.685 mmol), potassium carbonate (194 mg, 1.405 mmol), copper(I) iodide (19.6 mg, 0.103 mmol), trans-N,N′-dimethylcyclohexane-1,2-diamine (0.032 mL, 0.206 mmol) and a solution of 3-((2-(trimethylsilyl)ethoxy)methyl)dihydropyrimidine-2,4(1H,3H)-dione (may be prepared as described in Description 1; 183 mg, 0.747 mmol), in anhydrous 1,4-dioxane (7.7 mL) were mixed in a microwave vial, flushed with nitrogen, sealed and heated at 140° C. After 18 h, the reaction was cooled to room temperature, filtered through filter paper, evaporated in vacuo and diluted with DCM (10 mL) and water (10 mL). The layers were separated, and the aqueous was extracted with DCM (10 mL). The organics were combined, dried, concentrated, dissolved in minimal DCM and loaded onto a cyclohexane-preconditioned 12 g Redisep silica column, eluting with 0-50% EtOAc in cyclohexane to give tert-butyl(S)-3-(4-(2,4-dioxo-3-((2-(trimethylsilyl)ethoxy)methyl)tetrahydropyrimidin-1(2H)-yl)-1H-indol-1-yl)piperidine-1-carboxylate (184 mg, 0.339 mmol, 49.5% yield). LCMS (high pH A): Rt=1.46 min, MNH₄⁺=560.2

### Description 297

### tert-Butyl 2-(4-bromo-1H-indol-1-yl)-7-azaspiro[3.5]nonane-7-carboxylate (D297)

4-Bromo-1H-indole (0.3 mL, 2.392 mmol), tert-butyl 2-hydroxy-7-azaspiro[3.5]nonane-7-carboxylate (1732 mg, 7.18 mmol) and 2-(triphenyl-I5-phosphaneylidene)acetonitrile (1441 mg, 4.78 mmol) in toluene (5.1 mL) were heated to 110° C. After 66 h, the reaction was allowed to cool to room temperature, concentrated, dissolved in minimal DCM and loaded onto a cyclohexane-preconditioned 110 g SNAP amino D silica column, and eluting with 0-20% EtOAc in cyclohexane to give tert-butyl 2-(4-bromo-1H-indol-1-yl)-7-azaspiro[3.5]nonane-7-carboxylate (642 mg, 1.531 mmol, 64.0% yield). LCMS (high pH A): Rt=1.60 min, MH⁺=419.1, 421.1

### Description 298

### tert-Butyl 2-(4-(2,4-dioxo-3-((2-(trimethylsilyl)ethoxy)methyl)tetrahydropyrimidin-1(2H)-yl)-1H-indol-1-yl)-7-azaspiro[3.5]nonane-7-carboxylate (D298)

tert-Butyl 2-(4-bromo-1H-indol-1-yl)-7-azaspiro[3.5]nonane-7-carboxylate (may be prepared as described in Description 297; 636 mg, 1.587 mmol), potassium carbonate (430 mg, 3.11 mmol), copper(I) iodide (43.3 mg, 0.227 mmol), trans-N,N′-dimethylcyclohexane-1,2-diamine (71.7 μL, 0.455 mmol) and 3-((2-(trimethylsilyl)ethoxy)methyl)dihydropyrimidine-2,4(1H,3H)-dione (may be prepared as described in Description 1; 404 mg, 1.653 mmol) in anhydrous 1,4-dioxane (17 mL) were mixed in a microwave vial, flushed with nitrogen, sealed and heated at 140° C. After 18 h, the reaction was cooled to room temperature, filtered through filter paper, evaporated in vacuo and diluted with DCM (20 mL) and water (20 mL). The layers were separated, and the aqueous was extracted with DCM (20 mL). The organics were combined, dried, concentrated, dissolved in minimal DCM and loaded onto a cyclohexane-preconditioned 40 g Redisep silica column, eluting with 0-40% EtOAc in cyclohexane to give tert-butyl 2-(4-(2,4-dioxo-3-((2-(trimethylsilyl)ethoxy)methyl)tetrahydropyrimidin-1(2H)-yl)-1H-indol-1-yl)-7-azaspiro[3.5]nonane-7-carboxylate (663 mg, 1.138 mmol, 75% yield). LCMS (high pH A): Rt=1.53 min, MH⁺=583.2

### Description 299

### 5-Bromo-N-((1r,4r)-4-(3-chloro-4-cyanophenoxy)cyclohexyl)picolinamide (D299)

A mixture of 5-bromopicolinic acid (250 mg, 1.238 mmol), 4-(((1r,4r)-4-aminocyclohexyl)oxy)-2-chlorobenzonitrile (may be prepared as described in Description 46; 372 mg, 1.485 mmol), diisopropylethylamine (320 mg, 0.432 mL, 2.475 mmol) and HATU (565 mg, 1.485 mmol) in DMF (5 mL) was stirred at room temperature for 65 hours. Saturated sodium bicarbonate solution (15 mL) was added, and the mixture was extracted with dichloromethane (3×10 mL). The combined extracts were washed with water (10 mL) and brine (10 mL). The organic phase was dried, concentrated and chromatographed, eluting with 0-60% ethyl acetate in cyclohexane to give 5-bromo-N-((1r,4r)-4-(3-chloro-4-cyanophenoxy)cyclohexyl)picolinamide (344 mg, 0.791 mmol, 63.9% yield). LCMS (high pH A): Rt=1.31 min, MH−=434.1

### Description 300

### N-((1r,4r)-4-(3-Chloro-4-cyanophenoxy)cyclohexyl)-5-(4-(hydroxymethyl)piperidin-1-yl)picolinamide (D300)

A mixture of 5-bromo-N-((1r,4r)-4-(3-chloro-4-cyanophenoxy)cyclohexyl)picolinamide (may be prepared as described in Description 299; 250 mg, 0.575 mmol), piperidin-4-ylmethanol (132 mg, 1.150 mmol), sodium tert-butoxide (111 mg, 1.150 mmol), DavePhos (45 mg, 20 mol %), and tris(dibenzylideneacetone)dipalladium(0) (57 mg, 10 mol %) in dry dioxane (5 mL) was stirred at 80° C. under nitrogen. After 18 hours, the mixture was cooled to room temperature, partitioned between water (15 mL) and dichloromethane (20 mL) and filtered through Celite. The organic phase was separated, dried, concentrated and chromatographed, eluting with 50-100% ethyl acetate in cyclohexane, then 0-10% ethanol in ethyl acetate to give N-((1r,4r)-4-(3-Chloro-4-cyanophenoxy)cyclohexyl)-5-(4-(hydroxymethyl)piperidin-1-yl)picolinamide (59 mg, 0.126 mmol, 21.9% yield). LCMS (high pH A): Rt=1.13 min, MH⁺=469.1

### Description 301

### N-((1r,4r)-4-(3-chloro-4-cyanophenoxy)cyclohexyl)-5-(4-formylpiperidin-1-yl)picolinamide (D301)

Dess-Martin periodinane (109 mg, 0.256 mmol) was added to a solution of N-((1r,4r)-4-(3-chloro-4-cyanophenoxy)cyclohexyl)-5-(4-(hydroxymethyl)piperidin-1-yl)picolinamide (may be prepared as described in Description 300; 80 mg, 0.171 mmol) in dichloromethane (5 mL). The reaction was stirred at room temperature for 2 h, diluted with dichloromethane (5 mL) and washed with saturated sodium bicarbonate solution (10 mL). The aqueous phase was extracted with dichloromethane (5 mL) and the combined organics were dried and evaporated to give N-((1r,4r)-4-(3-chloro-4-cyanophenoxy)cyclohexyl)-5-(4-formylpiperidin-1-yl)picolinamide (80 mg, 0.171 mmol, 100% yield). LCMS (high pH A): Rt=1.20 min, MH⁺=467.1

### Description 302

### 4-Bromo-N-((1r,4r)-4-(3-chloro-4-cyanophenoxy)cyclohexyl)-2-fluorobenzamide (D302)

A mixture of 5-bromopicolinic acid (4-bromo-2-fluorobenzoic acid (250 mg, 1.142 mmol), 4-(((1r,4r)-4-aminocyclohexyl)oxy)-2-chlorobenzonitrile (Description 46; 343 mg, 1.370 mmol), diisopropylethylamine (295 mg, 0.399 mL, 2.283 mmol) and HATU (521 mg, 1.370 mmol) in DMF (3 mL) was stirred at room temperature for 1 h. Saturated sodium bicarbonate solution (10 mL) was added, and the mixture was extracted with EtOAc (2×10 mL). The combined extracts were washed with 2M hydrochloric acid (5 mL), water (5 mL) and brine (5 mL). The organic phase was dried, concentrated and chromatographed, eluting with 0-60% ethyl acetate in cyclohexane to give 4-bromo-N-((1r,4r)-4-(3-chloro-4-cyanophenoxy)cyclohexyl)-2-fluorobenzamide (465 mg, 1.029 mmol, 90% yield). LCMS (high pH A): Rt=1.32 min, MH⁺=452.8

### Description 303

### N-((1r,4r)-4-(3-Chloro-4-cyanophenoxy)cyclohexyl)-2-fluoro-4-(4-(hydroxymethyl)piperidin-1-yl)benzamide (D303)

A mixture of 4-bromo-N-((1r,4r)-4-(3-chloro-4-cyanophenoxy)cyclohexyl)-2-fluorobenzamide (may be prepared as described in Description 302; 250 mg, 0.553 mmol), piperidin-4-ylmethanol (127 mg, 1.107 mmol), sodium tert-butoxide (106 mg, 1.107 mmol), DavePhos (45 mg, 20 mol %), and tris(dibenzylideneacetone)dipalladium(0) (54 mg, 10 mol %) in dry dioxane (5 mL) was stirred at 80° C. under nitrogen. After 18 hours, the mixture was cooled to room temperature, partitioned between water (10 mL) and dichloromethane (10 mL) and filtered through Celite. The organic phase was separated, dried, concentrated and chromatographed, eluting with 50-100% ethyl acetate in cyclohexane, then 0-10% ethanol in ethyl acetate to give N-((1r,4r)-4-(3-chloro-4-cyanophenoxy)cyclohexyl)-2-fluoro-4-(4-(hydroxymethyl)piperidin-1-yl)benzamide (66 mg, 0.136 mmol, 24.54% yield). LCMS (high pH A): Rt=1.19 min, MH⁺=486.1, 488.0

### Description 304

### N-((1r,4r)-4-(3-Chloro-4-cyanophenoxy)cyclohexyl)-2-fluoro-4-(4-formylpiperidin-1-yl)benzamide (D304)

Dess-Martin periodinane (85 mg, 0.201 mmol) was added to a solution of N-((1r,4r)-4-(3-chloro-4-cyanophenoxy)cyclohexyl)-2-fluoro-4-(4-(hydroxymethyl)piperidin-1-yl)benzamide (may be prepared as described in Description 303; 65 mg, 0.134 mmol) in dichloromethane (5 mL). The reaction was stirred at room temperature for 2 h, diluted with dichloromethane (5 mL) and washed with saturated sodium bicarbonate solution (10 mL). The aqueous phase was extracted with dichloromethane (5 mL) and the combined organics were dried and evaporated to give N-((1r,4r)-4-(3-chloro-4-cyanophenoxy)cyclohexyl)-2-fluoro-4-(4-formylpiperidin-1-yl)benzamide (64 mg, 0.132 mmol, 99% yield). LCMS (high pH A): Rt=1.26 min, MH⁺=484.0, 486.0

### Description 305

### tert-Butyl(S)-3-((6-(((1r,4S)-4-(3-chloro-4-cyanophenoxy)cyclohexyl)carbamoyl)pyridazin-3-yl)oxy)pyrrolidine-1-carboxylate (D305)

To tert-butyl(S)-3-hydroxypyrrolidine-1-carboxylate (141 mg, 0.755 mmol) and 60% NaH in mineral oil (32.1 mg, 0.802 mmol) cooled in an ice-water bath was added DMF (3 ml). After 20 mins, 6-chloro-N-((1r,4r)-4-(3-chloro-4-cyanophenoxy)cyclohexyl)pyridazine-3-carboxamide (may be prepared as described in Description 90; 196 mg, 0.501 mmol) was added, and the reaction was for 20 h whilst warming to room temperature. The reaction mixture was diluted with water (10 ml) and EtOAc (10 ml), and the layers were separated. The organic layer was washed with water (10 ml), passed through a hydrophobic frit, concentrated in vacuo, dissolved in a minimal volume DCM and loaded onto a 24 g silica column, eluting with 0-80% EtOAc in cyclohexane to give tert-butyl(S)-3-((6-(((1r,4S)-4-(3-chloro-4-cyanophenoxy)cyclohexyl)carbamoyl)pyridazin-3-yl)oxy)pyrrolidine-1-carboxylate (267 mg, 0.493 mmol, 98% yield). LCMS (high pH A): Rt=1.35 min, M-BOC⁺=442.0, 444.0

### Description 306

### N-((1r,4S)-4-(3-chloro-4-cyanophenoxy)cyclohexyl)-6-(((S)-pyrrolidin-3-yl)oxy)pyridazine-3-carboxamide (D306)

To tert-butyl(S)-3-((6-(((1r,4S)-4-(3-chloro-4-cyanophenoxy)cyclohexyl)carbamoyl)pyridazin-3-yl)oxy)pyrrolidine-1-carboxylate (may be prepared as described in Description 305; 267 mg, 0.493 mmol) in DCM (5 mL) was added TFA (1.518 mL, 19.70 mmol). After 6 h, the reaction was concentrated, dissolved in DCM (20 mL) and diluted with sat. aq. sodium hydrogen carbonate solution (20 mL). The isolated aqueous layer was extracted with DCM (20 mL), and the combined organic layers were filtered through a hydrophobic frit and concentrated to give N-((1r,4S)-4-(3-chloro-4-cyanophenoxy)cyclohexyl)-6-(((S)-pyrrolidin-3-yl)oxy)pyridazine-3-carboxamide (198 mg, 0.448 mmol, 91% yield). LCMS (high pH A): Rt=1.06 min, MH⁺=442.0, 444.0

### Description 307

### tert-Butyl 4-(1-(4-bromo-1H-indol-1-yl)ethyl)piperidine-1-carboxylate (D307)

A mixture of 4-bromo-1H-indole (0.30 mL, 2.392 mmol), tert-butyl 4-(1-hydroxyethyl)piperidine-1-carboxylate (1.55 g, 6.76 mmol) and 2-(triphenyl-I5-phosphaneylidene)acetonitrile (1.441 g, 4.78 mmol) in toluene (5.10 mL) was heated to 110° C. After 18 h, the reaction was allowed to cool to room temperature, concentrated, dissolved in DCM (5 mL) and loaded onto a cyclohexane-preconditioned 110 g SNAP amino D silica column, eluting with 0-15% EtOAc in cyclohexane to give tert-butyl 4-(1-(4-bromo-1H-indol-1-yl)ethyl)piperidine-1-carboxylate (177 mg, 0.435 mmol, 18.17% yield). LCMS (high pH A): Rt=1.56 min, M-tBu⁺=351.1, 353.0

### Description 308

### tert-Butyl 4-(1-(4-(2,4-dioxo-3-((2-(trimethylsilyl)ethoxy)methyl)tetrahydropyrimidin-1(2H)-yl)-1H-indol-1-yl)ethyl)piperidine-1-carboxylate (D308)

3-((2-(Trimethylsilyl)ethoxy)methyl)dihydropyrimidine-2,4(1H,3H)-dione (may be prepared as described in Description 1; 130 mg, 0.532 mmol), potassium carbonate (138 mg, 1.001 mmol), copper(I) iodide (13.96 mg, 0.073 mmol), trans-N,N′-dimethylcyclohexane-1,2-diamine (0.023 mL, 0.147 mmol) and a solution of tert-butyl 4-(1-(4-bromo-1H-indol-1-yl)ethyl)piperidine-1-carboxylate (may be prepared as described in Description 307; 199 mg, 0.489 mmol) in anhydrous 1,4-dioxane (5.5 mL) were mixed in a microwave vial, sealed, bubbled with nitrogen and heated at 140° C. After 18 h the reaction was cooled to room temperature, filtered through filter paper concentrated in vacuo and diluted with DCM (15 mL) and water (15 mL). The layers were separated and the aqueous extracted with DCM (15 mL). The organics were combined, dried by passing through a hydrophobic frit, concentrated, dissolved in minimal DCM and loaded onto a cyclohexane preconditioned 12 g Redisep silica column, eluting with 0-50% EtOAc in cyclohexane to give tert-butyl 4-(1-(4-(2,4-dioxo-3-((2-(trimethylsilyl)ethoxy)methyl)tetrahydropyrimidin-1(2H)-yl)-1H-indol-1-yl)ethyl)piperidine-1-carboxylate (164 mg, 0.287 mmol, 58.8% yield). LCMS (high pH A): Rt=1.50 min, MNH₄⁺=588.2

### Description 309

### N-((1r,4r)-4-(3-Chloro-4-cyanophenoxy)cyclohexyl)-6-(2-hydroxy-7-azaspiro[3.5]nonan-7-yl)pyridazine-3-carboxamide (D309)

6-Chloro-N-((1r,4r)-4-(3-chloro-4-cyanophenoxy)cyclohexyl)pyridazine-3-carboxamide (may be prepared as described in Description 90; 200 mg, 0.511 mmol), 7-azaspiro[3.5]nonan-2-ol hydrochloride (109 mg, 0.613 mmol) and diisopropylethylamine (198 mg, 0.268 mL, 1.534 mmol) in dimethyl sulfoxide (3 mL) were heated at 80° C. After 20 h, the mixture was cooled to room temperature and partitioned between ethyl acetate (15 mL) and water (15 mL). The organic phase was separated, and the aqueous phase was extracted with ethyl acetate (2×5 mL). The combined organics were washed with brine (10 mL), dried, concentrated and chromatographed, eluting with 50-100% ethyl acetate in cyclohexane, then 0-10% ethanol in ethyl acetate to give N-((1r,4r)-4-(3-chloro-4-cyanophenoxy)cyclohexyl)-6-(2-hydroxy-7-azaspiro[3.5]nonan-7-yl)pyridazine-3-carboxamide (225 mg, 0.454 mmol, 89% yield). LCMS (high pH A): Rt=1.12 min, MH⁺=496.1, 498.0

### Description 310

### N-((1r,4r)-4-(3-Chloro-4-cyanophenoxy)cyclohexyl)-6-(2-oxo-7-azaspiro[3.5]nonan-7-yl)pyridazine-3-carboxamide (D310)

Dess-Martin periodinane (141 mg, 0.333 mmol) and N-((1r,4r)-4-(3-chloro-4-cyanophenoxy)cyclohexyl)-6-(2-hydroxy-7-azaspiro[3.5]nonan-7-yl)pyridazine-3-carboxamide (may be prepared as described in Description 309; 110 mg, 0.222 mmol) in dichloromethane (2 mL). were stirred for 1 h. Saturated sodium bicarbonate solution (2 mL) was added, and the mixture was stirred for 5 minutes. The organic layer was separated, and the aqueous phase was extracted with dichloromethane (2×2 mL). The combined organics were dried and evaporated to give N-((1r,4r)-4-(3-chloro-4-cyanophenoxy)cyclohexyl)-6-(2-oxo-7-azaspiro[3.5]nonan-7-yl)pyridazine-3-carboxamide (110 mg, 0.222 mmol, 100% yield). LCMS (high pH A): Rt=1.20 min, MH⁺=494.1, 496.1

### Description 311

### Methyl (1s,4s)-4-(tosyloxy)cyclohexane-1-carboxylate (D311)

To DMAP (0.382 g, 3.12 mmol)methyl (1s,4s)-4-hydroxycyclohexane-1-carboxylate (4.94 g, 31.2 mmol) and tosyl-Cl (17.86 g, 94 mmol) in DCM (150 mL) was added triethylamine (15.23 mL, 109 mmol). After 17 h, the mixture was diluted with water (150 mL), the layers were separated, and the aqueous layer was extracted with EtOAc (150 mL). The combined organic layers were filtered through a hydrophobic frit, concentrated and purified through a 120 g silica column, eluting with 0-50% EtOAc in cyclohexane. Product-containing fractions were combined and concentrated, and the residue was adsorbed onto Celite and repurified using the same conditions to give methyl (1s,4s)-4-(tosyloxy)cyclohexane-1-carboxylate (5.02 g, 16.07 mmol, 51.5% yield). LCMS (high pH A): Rt=1.14 min, MH₂O⁺=329.9

### Description 312

### Methyl (1r,4r)-4-(4-bromo-1H-indol-1-yl)cyclohexane-1-carboxylate (D312)

To 4-bromo-1H-indole (0.952 mL, 7.59 mmol) and cesium carbonate (4.94 g, 15.17 mmol) in NMP (15 mL) was added methyl (1s,4s)-4-(tosyloxy)cyclohexane-1-carboxylate (may be prepared as described in Description 311; 2.37 g, 7.59 mmol). The reaction was heated to 100° C. for 3 h, cooled to room temperature and diluted with water (75 mL) and EtOAc (75 mL). The layers were separated. The organic layer was washed with water (75 mL), filtered through a hydrophobic frit and purified using a 120 g silica column, eluting with 0-100% EtOAc in cyclohexane to give partially pure methyl (1r,4r)-4-(4-bromo-1H-indol-1-yl)cyclohexane-1-carboxylate (2.00 g, 5.95 mmol, 78% yield). LCMS (high pH A): Rt=1.39 min, MH⁺=337.8

### Description 313

### (1r,4r)-4-(4-Bromo-1H-indol-1-yl)cyclohexyl)methanol (D313)

To methyl (1r,4r)-4-(4-bromo-1H-indol-1-yl)cyclohexane-1-carboxylate (may be prepared as described in Description 312; 2.59 g, 7.70 mmol) in anhydrous THF (100 mL) at 0° C. was added DIBAL-H (25 weight % in toluene) (12.95 mL, 19.26 mmol), followed by stirring for 4 h while warming to room temperature. The reaction was cooled in an ice-water bath, and water (100 mL) was carefully added. The THF was removed in vacuo, and the mixture was diluted with water (50 mL) and EtOAc (150 mL), and the layers were separated. The aqueous layer was extracted with EtOAc (150 mL), and the combined organic layers were filtered through a hydrophobic frit, concentrated, dissolved in minimum DCM and loaded onto an 80 g silica column, eluting with 0-100% EtOAc in cyclohexane to give ((1r,4r)-4-(4-bromo-1H-indol-1-yl)cyclohexyl)methanol (288 mg, 0.934 mmol, 12.13% yield). LCMS (high pH A): Rt=1.23 min, MH⁺=309.9

### Description 314

### 1-(1-((1r,4r)-4-(Hydroxymethyl)cyclohexyl)-1H-indol-4-yl)-3-((2-(trimethylsilyl)ethoxy)methyl)dihydropyrimidine-2,4(1H,3H)-dione (D314)

To copper(I) iodide (71.2 mg, 0.374 mmol), potassium carbonate (258 mg, 1.869 mmol), 3-((2-(trimethylsilyl)ethoxy)methyl)dihydropyrimidine-2,4(1H,3H)-dione (may be prepared as described in Description 1; 274 mg, 1.121 mmol), ((1r,4r)-4-(4-bromo-1H-indol-1-yl)cyclohexyl)methanol (may be prepared as described in Description 313; 288 mg, 0.934 mmol) and trans-N,N′-dimethylcyclohexane-1,2-diamine (0.059 mL, 0.374 mmol) under nitrogen in a sealed vial was added anhydrous 1,4-dioxane (5 mL). The mixture was stirred at 140° C. in the sealed vessel for 18 h, cooled to room temperature, filtered through Celite, concentrated, dissolved in minimum DCM and purified using a 24 g silica column, eluting with 0-100% EtOAc in cyclohexane to give 1-(1-((1r,4r)-4-(hydroxymethyl)cyclohexyl)-1H-indol-4-yl)-3-((2-(trimethylsilyl)ethoxy)methyl)dihydropyrimidine-2,4(1H,3H)-dione (258 mg, 0.547 mmol, 58.5% yield). LCMS (high pH A): Rt=1.22 min, MH⁺=472.1

### Description 315

### (1r,4r)-4-(4-(2,4-Dioxo-3-((2-(trimethylsilyl)ethoxy)methyl)tetrahydropyrimidin-1(2H)-yl)-1H-indol-1-yl)cyclohexane-1-carbaldehyde (D315)

Dess-Martin periodinane (348 mg, 0.820 mmol) was added to 1-(1-((1r,4r)-4-(hydroxymethyl)cyclohexyl)-1H-indol-4-yl)-3-((2-(trimethylsilyl)ethoxy)methyl)dihydropyrimidine-2,4(1H,3H)-dione (may be prepared as described in Description 314; 258 mg, 0.547 mmol) in DCM (6 mL). After 3 h, the mixture was diluted with DCM (5 mL), 10% aq. sodium thiosulfate solution (5 mL) and sat. aq. sodium bicarbonate solution (5 mL), and the mixture was stirred vigorously for 5 min. The layers were separated, and the organic layer was filtered through a hydrophobic frit concentrated, dissolved in minimum DCM and loaded onto a 24 g silica column, eluting with 0-100% EtOAc in cyclohexane, to give (1r,4r)-4-(4-(2,4-dioxo-3-((2-(trimethylsilyl)ethoxy)methyl)tetrahydropyrimidin-1(2H)-yl)-1H-indol-1-yl)cyclohexane-1-carbaldehyde (115 mg, 0.245 mmol, 44.8% yield). LCMS (high pH A): Rt=1.29 min, MH⁺=470.1

### Description 316

### N-((1r,4r)-4-((3-Chloro-4-cyanophenyl)(methyl)amino)cyclohexyl)-6-(4-(hydroxymethyl)piperidin-1-yl)pyridazine-3-carboxamide (D316)

6-Chloro-N-((1r,4r)-4-((3-chloro-4-cyanophenyl)(methyl)amino)cyclohexyl)pyridazine-3-carboxamide (may be prepared as described in Description 243; 200 mg, 0.495 mmol), 4-hydroxymethylpiperidine (85 mg, 0.742 mmol) and DIPEA (0.259 mL, 1.484 mmol) were heated in DMSO (5 ml) at 100° C. After 2 h, the mixture was cooled, diluted with water (20 ml) and extracted with EtOAc (2×20 ml). The combined organics were washed with water, dried and evaporated in vacuo to give N-((1r,4r)-4-((3-chloro-4-cyanophenyl)(methyl)amino)cyclohexyl)-6-(4-(hydroxymethyl)piperidin-1-yl)pyridazine-3-carboxamide (230 mg, 0.476 mmol, 96% yield). LCMS (high pH A): Rt=1.10 min, MH⁺=483.2, 485.1

### Description 317

### N-((1r,4r)-4-((3-Chloro-4-cyanophenyl)(methyl)amino)cyclohexyl)-6-(4-formylpiperidin-1-yl)pyridazine-3-carboxamide (D317)

Dess-Martin periodinane (274 mg, 0.646 mmol) was added to N-((1r,4r)-4-((3-chloro-4-cyanophenyl)(methyl)amino)cyclohexyl)-6-(4-(hydroxymethyl)piperidin-1-yl)pyridazine-3-carboxamide (may be prepared as described in Description 316; 240 mg, 0.497 mmol) in DCM (20 ml). After 1 h the reaction was quenched with 10% sodium thiosulphate solution (20 ml) and stirred for 10 minutes. Sodium bicarbonate (saturated aqueous 20 ml) was added, and the mixture stirred for 20 minutes. The phases were separated, and the organic layer was dried through a hydrophobic frit and evaporated in vacuo to give N-((1r,4r)-4-((3-chloro-4-cyanophenyl)(methyl)amino)cyclohexyl)-6-(4-formylpiperidin-1-yl)pyridazine-3-carboxamide (210 mg, 0.437 mmol, 88% yield). LCMS (high pH A): Rt=1.18 min, MH⁺=481.1, 483.1

### Description 318

### Benzyl 4-(4-(2,4-dioxotetrahydropyrimidin-1(2H)-yl)-6-fluoro-1H-indol-1-yl)piperidine-1-carboxylate (D318)

To benzyl 4-(4-(2,4-dioxo-3-((2-(trimethylsilyl)ethoxy)methyl)tetrahydropyrimidin-1(2H)-yl)-6-fluoro-1H-indol-1-yl)piperidine-1-carboxylate (may be prepared as described in Description 10; 5.28 g, 8.88 mmol) in DCM (50 mL) was added trifluoroacetic acid (10.26 mL, 133 mmol). After 3 h the reaction was concentrated and dissolved in methanol (20 mL), followed by the addition of ammonia (7 M in methanol) (25.4 mL, 178 mmol). After 1.5 h, DCM (100 mL) was added, and mixture was diluted with saturated aqueous sodium hydrogen carbonate (75 mL). The layers were separated, and the aqueous layer was extracted with DCM (75 ml). The combined organic layers were filtered through a hydrophobic frit, concentrated, adsorbed onto Florisil and purified using a 120 g silica column, eluting with 50-100% EtOAc in cyclohexane (product eluted at 60-70%) to give benzyl 4-(4-(2,4-dioxotetrahydropyrimidin-1(2H)-yl)-6-fluoro-1H-indol-1-yl)piperidine-1-carboxylate (2.63 g, 5.66 mmol, 63.8% yield). LCMS (high pH A): Rt=1.11 min, MH⁺=482.2

### Description 319

### 4-Bromo-N-((1r,4r)-4-(3-chloro-4-cyanophenoxy)cyclohexyl)-3-fluorobenzamide (D319)

4-Bromo-3-fluorobenzoic acid (1.141 g, 5.21 mmol), 4-(((1r,4r)-4-aminocyclohexyl)oxy)-2-chlorobenzonitrile (may be prepared as described in Description 46; 1.306 g, 5.21 mmol), diisopropylethylamine (1.820 mL, 10.42 mmol) and HATU (2.377 g, 6.25 mmol) in DMF (3 mL) were stirred. After 3 hours, saturated sodium bicarbonate solution (10 mL) was added, and the mixture was extracted with ethyl acetate (2×10 mL). The combined extracts were washed with 2M hydrochloric acid (5 mL), water (5 ml) and brine (5 mL). The organic phase was dried, concentrated and purified over silica, eluting with 0-50% ethyl acetate in cyclohexane to give 4-bromo-N-((1r,4r)-4-(3-chloro-4-cyanophenoxy)cyclohexyl)-3-fluorobenzamide (444 mg, 0.983 mmol, 18.87% yield). LCMS (high pH A): Rt=1.31 min, M−H=451.1

### Description 320

### N-((1r,4r)-4-(3-Chloro-4-cyanophenoxy)cyclohexyl)-3-fluoro-4-(4-(hydroxymethyl)piperidin-1-yl)benzamide (D320)

To 4-bromo-N-((1r,4r)-4-(3-chloro-4-cyanophenoxy)cyclohexyl)-3-fluorobenzamide (may be prepared as described in Description 319; 437 mg, 0.967 mmol), piperidin-4-ylmethanol (229 mg, 1.988 mmol) and sodium tert-butoxide (189 mg, 1.967 mmol) in anhydrous 1,4-dioxane (5 mL) were added tris(dibenzylideneacetone)dipalladium(0) (91 mg, 0.099 mmol) and DavePhos (77 mg, 0.196 mmol), and the reaction was heated to 80° C. After 22.25 h, the mixture was cooled to room temperature, partitioned between water (10 mL) and dichloromethane (10 mL), and filtered through Celite. The organic phase was dried, concentrated and chromatographed, eluting with 50-100% ethyl acetate in cyclohexane, then 0-10% ethanol in ethyl acetate. Impure product was re-choreographed, eluting with 0-5% EtOH in DCM to give N-((1r,4r)-4-(3-chloro-4-cyanophenoxy)cyclohexyl)-3-fluoro-4-(4-(hydroxymethyl)piperidin-1-yl)benzamide (82 mg, 0.169 mmol, 17.44% yield). LCMS (high pH A): Rt=1.16 min, MH⁺=486.1, 488.1

### Description 321

### N-((1r,4r)-4-(3-Chloro-4-cyanophenoxy)cyclohexyl)-3-fluoro-4-(4-formylpiperidin-1-yl)benzamide (D321)

N-((1r,4r)-4-(3-Chloro-4-cyanophenoxy)cyclohexyl)-3-fluoro-4-(4-(hydroxymethyl)piperidin-1-yl)benzamide (may be prepared as described in Description 320; 49 mg, 0.101 mmol) and Dess-Martin periodinane (62 mg, 0.146 mmol) were stirred in DCM (3 mL). After 1 h 40 minutes the reaction was partitioned between DCM (10 mL) and sodium bicarbonate (10 mL). The aqueous phase was extracted with DCM (5 mL×1), and the combined organic phases were washed with water (5 mL) and brine (5 mL). The organic phase was dried and evaporated to give N-((1r,4r)-4-(3-chloro-4-cyanophenoxy)cyclohexyl)-3-fluoro-4-(4-formylpiperidin-1-yl)benzamide (55 mg, 0.114 mmol, 113% yield). LCMS (high pH A): Rt=1.24 min, MH⁺=484.1, 486.1

### Description 322

### (3-(4-Bromoindolin-1-yl)cyclobutyl)methanol (D322)

To 3-(hydroxymethyl)cyclobutan-1-one (1.668 g, 16.66 mmol) and 4-bromoindoline (3.0 g, 15.15 mmol) in acetic acid (30 mL) cooled in an ice-water bath was added sodium triacetoxyborohydride (3.85 g, 18.18 mmol). The reaction was stirred at room temperature for 7 h, then added to vigorously stirred ice-cooled saturated NaHCO₃ (aq) (150 mL). The mixture was basified with 2 M NaOH (aq) to PH˜7 and extracted with EtOAc (2×100 mL). The organic extracts were combined, washed with brine (100 mL), passed through a hydrophobic frit, concentrated and loaded in DCM (6 mL) onto a 120 g silica cartridge, eluting with 0-70% 3:1 EtOAc:ethanol in TBME to give (3-(4-bromoindolin-1-yl)cyclobutyl)methanol (0.562 g, 1.992 mmol). LCMS (formic A): Rt=1.11 min, MH⁺=282, 284

### Description 323

### (3-(4-Bromo-1H-indol-1-yl)cyclobutyl)methanol (D323)

To the cis/trans mixture (3-(4-bromoindolin-1-yl)cyclobutyl)methanol (may be prepared as described in Description 322; 0.56 g, 1.985 mmol) in anhydrous 1,4-dioxane (12 mL) in an ice-water bath was added DDQ (0.676 g, 2.98 mmol). The mixture was allowed to warm to room temperature over 5 min and stirred for 22 h. Further DDQ (300 mg) was added, and after 6 h, the reaction was diluted with sat. NaHCO₃ (aq) (40 mL) and EtOAc (40 mL). The organic layer was separated, washed with brine (40 mL), passed through a hydrophobic frit, concentrated and loaded preabsorbed on Florisil (from methanol) onto an 80 g silica cartridge, eluting with 0-100% EtOAc in to give (3-(4-bromo-1H-indol-1-yl)cyclobutyl)methanol (187 mg, 0.667 mmol, 33.6% yield). LCMS (formic A): Rt=1.51 min, MH⁺=280, 282

### Description 324

### 1-(1-(3-(Hydroxymethyl)cyclobutyl)-1H-indol-4-yl)-3-((2-(trimethylsilyl)ethoxy)methyl)dihydropyrimidine-2,4(1H,3H)-dione (D324)

A mixture of copper(I) iodide (19 mg, 0.100 mmol), potassium carbonate (184 mg, 1.335 mmol) and 3-((2-(trimethylsilyl)ethoxy)methyl)dihydropyrimidine-2,4(1H,3H)-dione (may be prepared as described in Description 1; 196 mg, 0.801 mmol) was suspended in a solution of (3-(4-bromo-1H-indol-1-yl)cyclobutyl)methanol (may be prepared as described in Description 323; 187 mg, 0.667 mmol) (a mixture of cis and trans isomers) in anhydrous 1,4-dioxane (3.0 mL). trans-N,N′-Dimethylcyclohexane-1,2-diamine (0.032 mL, 0.200 mmol) was added, and the vessel was evacuated and purged with nitrogen (×3). The mixture was stirred at 120° C. for 27 h, allowed to cool to room temperature, and copper(I) iodide (40 mg) and trans-N,N′-dimethylcyclohexane-1,2-diamine (0.064 mL) were added. The vessel was evacuated and purged with nitrogen (×3). The mixture was stirred at 140° C. in the sealed vessel for 18 h. The reaction was cooled to room temperature, filtered through Celite (washing with EtOAc [8 mL]), concentrated and loaded in DCM (2 mL) onto a 40 g silica cartridge, eluting with 0-100% EtOAc in cyclohexane to give 1-(1-(3-(Hydroxymethyl)cyclobutyl)-1H-indol-4-yl)-3-((2-(trimethylsilyl)ethoxy)methyl)dihydropyrimidine-2,4(1H,3H)-dione (166 mg, 0.374 mmol, 56.1% yield). LCMS (formic A): Rt=1.15 min, MH⁺=444

### Description 325

### 3-(4-(2,4-Dioxo-3-((2-(trimethylsilyl)ethoxy)methyl)tetrahydropyrimidin-1(2H)-yl)-1H-indol-1-yl)cyclobutane-1-carbaldehyde (D325)

To 1-(1-(3-(hydroxymethyl)cyclobutyl)-1H-indol-4-yl)-3-((2-(trimethylsilyl)ethoxy)methyl)dihydropyrimidine-2,4(1H,3H)-dione (may be prepared as described in Description 324; 166 mg, 0.374 mmol) (a mixture of cis and trans isomers) in DCM (2 mL) was added DMP (190 mg, 0.449 mmol). After 1 h the mixture was diluted with DCM (5 mL) and stirred with sat. sodium thiosulfate (aq) (5 mL). After 5 min the organic layer was separated, washed with sat. NaHCO₃ (aq) (5 mL), passed through a hydrophobic frit, concentrated and loaded in DCM (3 mL) onto a 24 g silica cartridge, eluting with 0-100% EtOAc in cyclohexane to give 3-(4-(2,4-dioxo-3-((2-(trimethylsilyl)ethoxy)methyl)tetrahydropyrimidin-1(2H)-yl)-1H-indol-1-yl)cyclobutane-1-carbaldehyde (81 mg, 0.183 mmol, 49.0% yield). LCMS (formic A): Rt=1.22 min, M-C₂H₄⁺=414

### Description 326

### N-((1r,4r)-4-(3-Chloro-4-cyanophenoxy)cyclohexyl)-6-(4-(hydroxymethyl)-4-methylpiperidin-1-yl)pyridazine-3-carboxamide (D326)

To 6-chloro-N-((1r,4r)-4-(3-chloro-4-cyanophenoxy)cyclohexyl)pyridazine-3-carboxamide (may be prepared as described in Description 90; 250 mg, 0.639 mmol) and (4-methylpiperidin-4-yl)methanol hydrochloride (127 mg, 0.767 mmol) in DMSO (3 mL) at 60° C. was added DIPEA (0.335 mL, 1.917 mmol). After 4 days and 17.5 h the mixture was partitioned between ethyl acetate (10 mL) and water (10 mL). The aqueous layer was separated, and the organic layer was washed with water (15 mL) and brine (15 mL). The organic layer was dried, evaporated and chromatographed, eluting with 20-100% ethyl acetate in cyclohexane to give N-((1r,4r)-4-(3-chloro-4-cyanophenoxy)cyclohexyl)-6-(4-(hydroxymethyl)-4-methylpiperidin-1-yl)pyridazine-3-carboxamide (146 mg, 0.302 mmol, 47.2% yield). LCMS (high pH A): Rt=1.14 min, MH⁺=484.2, 486.1

### Description 327

### N-((1r,4r)-4-(3-Chloro-4-cyanophenoxy)cyclohexyl)-6-(4-formyl-4-methylpiperidin-1-yl)pyridazine-3-carboxamide (D327)

Dimethyl sulfoxide (0.293 mL, 4.13 mmol) was added to oxalyl dichloride (0.033 mL, 2.061 mmol) and 3 Å molecular sieves in DCM (1 mL) at −78° C. After 30 minutes, a solution of N-((1r,4r)-4-(3-chloro-4-cyanophenoxy)cyclohexyl)-6-(4-(hydroxymethyl)-4-methylpiperidin-1-yl)pyridazine-3-carboxamide (may be prepared as described in Description 326; 69 mg, 0.143 mmol) in DCM (1 mL) was added slowly. After 20 minutes, triethylamine (0.744 mL, 6.20 mmol) in DCM (1 mL) was added dropwise. The reaction was stirred for a further 15 minutes and then allowed to slowly warm up to room temperature over 25 minutes. The mixture was diluted with water (10 mL), and the organic layer was separated. The aqueous layer was extracted with DCM (2×5 mL). The combined organic layers were washed with water (5 mL) and brine (5 mL) to give N-((1r,4r)-4-(3-chloro-4-cyanophenoxy)cyclohexyl)-6-(4-formyl-4-methylpiperidin-1-yl)pyridazine-3-carboxamide (46 mg, 0.095 mmol, 66.9% yield). LCMS (high pH A): Rt=1.21 min, MH⁺=482.1, 484.1

### Description 328

### ((1r,4r)-4-(4-(2,4-Dioxo-3-((2-(trimethylsilyl)ethoxy)methyl)tetrahydropyrimidin-1(2H)-yl)-1H-indol-1-yl)cyclohexyl)methyl 4-methylbenzenesulfonate (D328)

To 1-(1-((1r,4r)-4-(hydroxymethyl)cyclohexyl)-1H-indol-4-yl)-3-((2-(trimethylsilyl)ethoxy)methyl)dihydropyrimidine-2,4(1H,3H)-dione (may be prepared as described in Description 314; 300 mg, 0.636 mmol) and tosyl-Cl (364 mg, 1.908 mmol) in DCM (5 mL) was added triethylamine (0.355 mL, 2.54 mmol). After 18 h the reaction was diluted with water (5 mL) and DCM (5 mL), and the layers were separated. The aqueous layer was extracted with DCM (5 ml), and the combined organic layers were filtered through a hydrophobic frit, concentrated, dissolved in minimum DCM and purified over a 24 g silica column, eluting with 0-50% EtOAc in cyclohexane to give ((1r,4r)-4-(4-(2,4-dioxo-3-((2-(trimethylsilyl)ethoxy)methyl)tetrahydropyrimidin-1(2H)-yl)-1H-indol-1-yl)cyclohexyl)methyl 4-methylbenzenesulfonate (269 mg, 0.430 mmol, 67.6% yield). LCMS (high pH A): Rt=1.47 min, M-Et⁺=598.1

### Description 329

### 3-(2,4,6-Trimethoxybenzyl)dihydropyrimidine-2,4(1H,3H)-dione (D329)

2,4,6-Trimethoxybenzylamine hydrochloride (10.2 g, 43.6 mmol) was dissolved DCM (40 mL) and neutralized with 2 M NaOH. The organic layer was separated, washed with brine, dried (MgSO₄), filtered and evaporated in vacuo to give 8.8 g of the free base, which was dissolved in DMF (100 mL) and treated with ethyl 3-isocyanatopropanoate (5.75 mL, 43.6 mmol). A modest exotherm occurred and the reaction mixture solidified. The reaction was warmed to effect solution formation, and the mixture was treated with NaH (3.49 g, 87 mmol) and stirred for 60 min. The reaction was heated at 100° C. for 30 min, evaporated to give a thick syrup which was dissolved in water and washed with ether. The aqueous layer was saturated with NaCl and extracted with ethyl acetate (3×). The organic extracts were combined, dried (MgSO₄), filtered and evaporated to give a white solid. This material was triturated with ether and dried in the vacuum oven at 40° C. overnight (ca 18 h) to give 3-(2,4,6-trimethoxybenzyl)dihydropyrimidine-2,4(1H,3H)-dione (8.6 g, 27.8 mmol, 63.6% yield). LCMS (high pH A): Rt=0.72 min, MH⁺=295.1

### Description 330

### 4-Bromo-1-methyl-1H-pyrrolo[3,2-c]pyridine (D330)

To 4-bromo-1H-pyrrolo[3,2-c]pyridine (295 mg, 1.497 mmol) in anhydrous NMP (3.0 mL) cooled in an ice-water bath was added sodium hydride (60% in mineral oil) (71.9 mg, 1.797 mmol). After ten min, the reaction was treated with methyl iodide (0.117 mL, 1.871 mmol). The mixture was allowed to stir in the ice bath for 10 min, then allowed to warm to rt and stir for 3 h. Sodium hydride (60% in mineral oil) (71.9 mg, 1.797 mmol) was added to the reaction, and the mixture was allowed to stir under nitrogen for 10 min. Methyl iodide (0.060 mL) was added, and the reaction stirred for 1 h. The reaction was quenched with water (3 mL), diluted with brine (3 mL) and extracted with EtOAc (2×15 mL). The organic extracts were combined and passed through a hydrophobic frit, evaporated and purified MDAP (high pH) to give 4-bromo-1-methyl-1H-pyrrolo[3,2-c]pyridine (236 mg, 1.118 mmol, 74.7%). LCMS (high pH A): Rt=0.85 min, MH⁺=211, 213

### Description D331

### 1-(2-Methoxyethyl)-4-nitro-1H-indole (D331)

To 4-nitro-1H-indole (1 g, 6.17 mmol) in DMF (10 mL) was added 1-bromo-2-methoxyethane (1.159 mL, 12.33 mmol), portion wise, and Cs₂CO₃ (5.02 g, 15.42 mmol). The reaction was stirred at 80° C. for 16 h, evaporated in vacuo, diluted with water (5 ml), and extracted with ethyl acetate (3×15 ml). The organic layers were washed with brine solution (5 ml), dried over sodium sulphate and evaporated in vacuo to give 1-(2-methoxyethyl)-4-nitro-1H-indole (1.38 g, 6.13 mmol, 97.9%). LCMS (formic A): Rt=0.97 min, MH⁺=221.2

### Description D332

### 1-(2-Methoxyethyl)-1H-indol-4-amine (D332)

1-(2-Methoxyethyl)-4-nitro-1H-indole (may be prepared as described in Description 331; 1.3 g, 5.90 mmol) in methanol (20 mL) was purged with nitrogen. Pd—C (0.628 g, 0.590 mmol) was added under nitrogen, and the resulting suspension was stirred at 30° C. under a hydrogen atmosphere (bladder) for 16 h. The mixture was filtered through a Celite pad, washing with methanol (3×5 mL). The filtrate was concentrated to give 1-(2-methoxyethyl)-1H-indol-4-amine (1.1 g, 5.67 mmol, 96%). LCMS (formic A): Rt=0.405 min, MH⁺=191.2

### Description D333

### 3,3′-((1-(2-Methoxyethyl)-1H-indol-4-yl) azanediyl)dipropionic acid (D333)

1-(2-Methoxyethyl)-1H-indol-4-amine (may be prepared as described in Description 332; 1.1 g, 5.78 mmol) in acrylic acid (0.794 ml, 11.56 mmol) was stirred at 27° C. for 8 hours, then concentrated to give 3,3′-((1-(2-methoxyethyl)-1H-indol-4-yl) azanediyl)dipropionic acid (1.6 g, 5.67 mmol, 96%). LCMS (formic A): Rt=0.473 min, MH⁺=335.0

### Description 334

### Methyl N-(2,6-dimethyl-3-nitrophenyl) formimidate (D334)

To 2,6-dimethyl-3-nitroaniline (3.1 g, 18.65 mmol) in trimethyl orthoformate (20.25 ml, 183 mmol) was added p-toluenesulfonic acid monohydrate (0.192 g, 1.007 mmol), and the reaction was heated to 120° C. After 1 hr the mixture was concentrated and purified over a RediSep C-18 column (120 g), eluting with MeCN in H₂O (0.1% NaHCO₃) to give methyl N-(2,6-dimethyl-3-nitrophenyl) formimidate (2.34 g, 10.46 mmol, 56.1% yield). LCMS (formic A): Rt=0.84 min, MH⁺=209.1

### Description 335

### 7-Methyl-4-nitro-1H-indole (D335)

To diethyl oxalate (1.901 mL, 13.92 mmol) in DMF (25 mL) at 0° C. was added 20% potassium ethoxide (5.86 g, 13.92 mmol) in ethanol, dropwise. The reaction was brought to room temperature and methyl N-(2,6-dimethyl-3-nitrophenyl) formimidate (may be prepared as described in Description 334; 2.1 g, 10.09 mmol) in DMF (25 mL) was added. After 16 h the reaction mixture was treated with ice cold water (200 mL) and extracted with EtOAc (2×150 mL). The combined organic layers were washed with brine (100 mL), dried over Na₂SO₄ (4 g), filtered and concentrated. The crude product was dissolved in DCM (7 mL), adsorbed on silica gel (1.5 g) and purified over silica (100 g), eluting with 0-100% EtOAc in pet. ether to afford 7-methyl-4-nitro-1H-indole (840 mg, 3.58 mmol, 35.5% yield). LCMS (formic A): Rt=0.87 min, MH⁻=175.0

### Description 336

### 1,7-Dimethyl-4-nitro-1H-indole (D336)

To 7-methyl-4-nitro-1H-indole (may be prepared as described in Description 335; 540 mg, 2.304 mmol) in DMF (15 mL) was added sodium hydride (92 mg, 2.304 mmol), followed after 30 min by methyl iodide (0.158 mL, 2.53 mmol). After 4 h ice cold water (50 mL) was added, and the resulting solid was collected by filtration to give 1,7-dimethyl-4-nitro-1H-indole (500 mg, 2.018 mmol, 88% yield). LCMS (formic A): Rt=0.97 min, MH⁺=191.2

### Description 337

### 1,7-Dimethyl-1H-indol-4-amine (D337)

1,7-Dimethyl-4-nitro-1H-indole (may be prepared as described in Description 336; 500 mg, 2.63 mmol) in ethyl acetate (20 mL) was purged with nitrogen and treated with 10% Pd—C (168 mg, 0.158 mmol) under nitrogen. The resulting suspension was stirred under hydrogen atmosphere (bladder) for 16 h, filtered through Celite, concentrated, dissolved in DCM (4 mL), adsorbed on silica gel (1.3 g) and purified over silica (50 g), eluting with 0-100% EtOAc in pet. ether to give 1,7-dimethyl-1H-indol-4-amine (250 mg, 1.482 mmol, 56.4% yield). LCMS (formic A): Rt=0.35 min, MH⁺=161.2

### Description 338

### 1-Ethyl-4-nitro-1H-indole (D338)

To 4-nitro-1H-indole (1.5 g, 9.25 mmol) in DMF (10 mL) was added ethyl iodide (1.495 mL, 18.50 mmol) and Cs₂CO₃ (7.54 g, 23.13 mmol). The reaction was stirred at 80° C. for 16 h, evaporated in vacuo, diluted with water (15 ml) and extracted with ethyl acetate (3×15 ml). The combined organic layers were washed with brine (10 ml), dried over sodium sulphate and evaporated in vacuo to give 1-ethyl-4-nitro-1H-indole (1.78 g, 9.17 mmol, 99% yield). LCMS (formic A): Rt=0.96 min, MH⁺=191.2

### Description 339

### 1-Ethyl-1H-indol-4-amine (D339)

1-Ethyl-4-nitro-1H-indole (may be prepared as described in Description 338; 1.78 g, 9.26 mmol) in methanol (20 mL) was purged with nitrogen, treated with Pd—C (0.986 g, 0.926 mmol) under nitrogen and stirred at 30° C. under hydrogen atmosphere (bladder). After 16 h the reaction was filtered through Celite, concentrated and purified over silica (40 g), eluting with 0-100% EtOAc in petroleum ether to give 1-ethyl-1H-indol-4-amine (695 mg, 4.33 mmol, 46.8% yield). LCMS (formic A): Rt=0.37 min, MH⁺=161.2

### Description 340

### 4-Bromo-1-ethyl-1H-pyrrolo[2,3-c]pyridine (D340)

To 4-bromo-1H-pyrrolo[2,3-c]pyridine (1 g, 5.08 mmol) in HMPA (8 mL) was added NaH (60%) (0.244 g, 6.09 mmol) at 0° C. After 1 h, iodoethane (0.615 mL, 7.61 mmol) was added, and the reaction was brought to room temperature. After 1 h, the mixture was quenched with ice and extracted with EtOAc (80 mL). Organic layer was separated, washed with brine (15 mL), dried over sodium sulfate, concentrated, dissolved in DCM (10 mL), adsorbed on silica gel (6 g) and purified over silica (25 g), eluting with 0-100% EtOAc in pet. ether to give 4-bromo-1-ethyl-1H-pyrrolo[2,3-c]pyridine (900 mg, 3.96 mmol, 78% yield). LCMS (formic A): Rt=0.38 min, MH⁺=225

### Description 341

### 1-(1-Ethyl-1H-pyrrolo[2,3-c]pyridin-4-yl)-3-((2-(trimethylsilyl)ethoxy)methyl)dihydropyrimidine-2,4(1H,3H)-dione (D341)

To 4-bromo-1-ethyl-1H-pyrrolo[2,3-c]pyridine (may be prepared as described in Description 340; 750 mg, 3.14 mmol), 3-((2-(trimethylsilyl)ethoxy)methyl)dihydropyrimidine-2,4(1H,3H)-dione (may be prepared as described in Description 1; 1150 mg, 4.70 mmol) and (1R,2R)—N1,N2-dimethylcyclohexane-1,2-diamine (446 mg, 3.14 mmol) in 1,4-dioxane (20 mL) at 25° C. was added Cs₂CO₃ (2044 mg, 6.27 mmol), and the mixture was degassed with nitrogen for 5 mins. Copper(I) iodide (299 mg, 1.568 mmol) was added, and the reaction was heated to 100° C. After 16 h, the mixture was filtered through Celite, rinsing with EtOAc (50 ml). Water (20 ml) was added, and the organic layer was separated, dried over Na₂SO₄, concentrated, dissolved in DCM (10 mL), adsorbed on silica gel (10 g) and purified over silica (40 g), eluting with 0-10% MeOH in DCM to give 1-(1-ethyl-1H-pyrrolo[2,3-c]pyridin-4-yl)-3-((2-(trimethylsilyl)ethoxy)methyl)dihydropyrimidine-2,4(1H,3H)-dione (1 g, 2.291 mmol, 64.4% yield). LCMS (formic A): Rt=0.698 min, MH⁺=388.8

### Description 342

### tert-Butyl ((1r,3r)-3-((8-cyanoquinolin-5-yl)oxy)-2,2,4,4-tetramethylcyclobutyl)carbamate (D342)

To a stirred solution of trans-tert-butyl 3-hydroxy-2,2,4,4-(tetramethyl)cyclobutylcarbamate (1.00 g, 1.00 Eq, 4.11 mmol) and 5-fluoroquinoline-8-carbonitrile (707 mg, 1.00 equivalents, 4.11 mmol) in MeCN (20 mL) was added cesium carbonate (3.35 g, 2.50 eq., 10.3 mmol) then the mixture heated at 100° C. for 5 h. DMF (10 mL) was added to aid solubility, then the mixture was heated at 100° C. for a further 20 h. The mixture was added to water (200 mL), then the solution was extracted with ethyl acetate (3×100 mL). The organic layers were combined, washed with brine, dried using a hydrophobic frit and evaporated in vacuo. The sample was loaded on florisil and purified on Si (80 g) using a 0-50% ethyl acetate-cyclohexane gradient over 12 column volumes. The appropriate fractions were combined and evaporated in vacuo to give the desired product (682 mg, 42% yield) as an off-white solid. LCMS (high pH A): Rt=1.33 min, MH⁺=396.

### Description 343

### 5-((1r,3r)-3-Amino-2,2,4,4-tetramethylcyclobutoxy)quinoline-8-carbonitrile, dihydrochloride (D343)

To a stirred solution of tert-butyl ((1r,3r)-3-((8-cyanoquinolin-5-yl)oxy)-2,2,4,4-tetramethylcyclobutyl)carbamate (may be prepared as described in Description 342; 680 mg, 1.72 mmol) in DCM (15 mL) was added HCl (4M in dioxane, 4.30 mL, 17.2 mmol) then the mixture stirred at rt for 66 h. The reaction mixture was concentrated in vacuo and dried to give the desired product (640 mg, quantitative yield) as a white solid. LCMS (high pH A): Rt=0.97 min, MH⁺=296.

### Description 344

### N-((1r,3r)-3-((8-Cyanoquinolin-5-yl)oxy)-2,2,4,4-tetramethylcyclobutyl)-6-(4-(hydroxymethyl)piperidin-1-yl)pyridazine-3-carboxamide (D344)

To a stirred solution of 5-((1r,3r)-3-amino-2,2,4,4-tetramethylcyclobutoxy)quinoline-8-carbonitrile, dihydrochloride (may be prepared as described in Description 343; 200 mg, 543 μmol) and 6-(4-(hydroxymethyl)piperidin-1-yl)pyridazine-3-carboxylic acid (may be prepared as described in Description 61, 279 mg, 1.06 mmol) in DCM (7.00 mL) was added HATU (310 mg, 814 μmol) and DIPEA (473 μL, 2.72 mmol) then the mixture stirred at room temperature for 2 h. The mixture was quenched with saturated NaHCO₃, diluted with ethyl acetate, and the organic layer was separated. The solvent was removed and the sample was purified by silicon column chromatography (4 g) using a 0-100% ethyl acetate-cyclohexane gradient. The appropriate fractions were combined and evaporated in vacuo to give the desired product (175 mg, 59% yield) as a white solid. LCMS (formic A): Rt=1.19 min, MH⁺=515.

### Description 345

### N-((1r,3r)-3-((8-Cyanoquinolin-5-yl)oxy)-2,2,4,4-tetramethylcyclobutyl)-6-(4-formylpiperidin-1-yl)pyridazine-3-carboxamide (D345)

N-((1r,3r)-3-((8-Cyanoquinolin-5-yl)oxy)-2,2,4,4-tetramethylcyclobutyl)-6-(4-(hydroxymethyl)piperidin-1-yl)pyridazine-3-carboxamide (may be prepared as described in Description 344; 157 mg, 305 μmol) was dissolved in DCM (6.0 mL) and cooled in an ice bath then Dess-Martin periodinane (168 mg, 397 μmol) was added, then the mixture was stirred at room temperature for 1 h. The reaction mixture was concentrated and DCM was added and the suspension was filtered. The organic layer was concentrated and purified by silicon chromatography (4 g) using a 0-75% ethyl acetate-cyclohexane gradient. The appropriate fractions were combined and evaporated to give the desired product (130 mg, 79% yield) as a white solid. LCMS (formic A): Rt=1.29 min, MH⁺=513.

### Description 346

### N-((1r,3r)-3-(3-Chloro-4-cyanophenoxy)-2,2,4,4-tetramethylcyclobutyl)-6-(4-(hydroxymethyl)piperidin-1-yl)pyridazine-3-carboxamide (D346)

To a stirred suspension of 6-(4-(hydroxymethyl)piperidin-1-yl)pyridazine-3-carboxylic acid (may be prepared as described in Description 61; 500 mg, 2.11 mmol) and 4-((1r,3r)-3-amino-2,2,4,4-tetramethylcyclobutoxy)-2-chlorobenzonitrile (may be prepared as described in Description 49; 664 mg, 2.11 mmol) in DCM (20 mL) was added DIPEA (1.09 g, 1.47 mL, 4.00 equivalents, 8.43 mmol) followed by HATU (841 mg, 1.05 equivalents, 2.21 mmol) then the mixture was stirred at room temperature for 30 min. The mixture was diluted with DCM (30 mL) and water (50 mL), the pH adjusted to 4 with HCl (2M aq.), then the phases were separated. The aqueous layer was back-extracted with DCM (2×50 mL), then the organic layers were combined, dried using a hydrophobic frit and evaporated in vacuo. The sample was purified using a 0-100% ethyl acetate-cyclohexane gradient over 12 column volumes. The appropriate fractions were combined and evaporated in vacuo to give the desired product (1.24 g, 85% purity, 83% yield) as a colourless gum that was used without further purification. LCMS (high pH A): Rt=1.28 min, MH⁺=498.

### Description 347

### N-((1r,3r)-3-(3-Chloro-4-cyanophenoxy)-2,2,4,4-tetramethylcyclobutyl)-6-(4-formylpiperidin-1-yl)pyridazine-3-carboxamide (D347)

To a stirred solution of N-((1r,3r)-3-(3-chloro-4-cyanophenoxy)-2,2,4,4-tetramethylcyclobutyl)-6-(4-formylpiperidin-1-yl)pyridazine-3-carboxamide (may be prepared as described in Description 346; 1.24 g, 85% purity, 1.74 mmol) in DCM (20.0 mL) was added DMP (1.11 g, 1.5 equivalents, 2.61 mmol) then the mixture was stirred at room temperature for 1 h. The reaction mixture was concentrated in vacuo, then the sample was purified by silicon chromatography (40 g) using a 0-100% ethyl acetate-cyclohexane gradient over 14 column volumes. The appropriate fractions were combined and evaporated in vacuo to give the desired product (677 mg, 78% yield) as a yellow gum which solidified. LCMS (high pH A): Rt=1.36 min, MH⁺=496.

### Description 348

### tert-Butyl ((1r,3r)-3-((5-chloro-6-cyanopyridin-3-yl)oxy)-2,2,4,4-tetramethylcyclobutyl)carbamate (D348)

A stirred solution of tert-butyl ((1r,3r)-3-hydroxy-2,2,4,4-tetramethylcyclobutyl)carbamate (3.00 g, 12.3 mmol) in DMF (41 mL) was cooled in an ice-water bath, then sodium hydride (592 mg, 60% w/w, 14.8 mmol) was added then the reaction mixture was stirred at room temperature for 40 min. The mixture was cooled to 0° C. then 3-chloro-5-fluoropicolinonitrile (2.12 g, 13.6 mmol) was added, then the ice bath was removed and the mixture stirred at room temperature for 22.5 h. The solution was partially concentrated in vacuo to one third of its initial volume. NH₄Cl (saturated aqueous, 50 ml) was added and extracted with ethyl acetate (3×70 ml). The combined organic layers were washed with brine and then dried over Na₂SO₄, filtered and the solvent removed under reduced pressure. The sample was purified by chromatography on Si (120 g) using a 15-100% ethyl acetate-cyclohexane gradient. The appropriate fractions were combined and evaporated in vacuo to give the desired product (2.90 g, 59% yield) as white solid. LCMS (formic A): Rt=0.91 min, MH⁺=380.

### Description 349

### 5-((1r,3r)-3-Amino-2,2,4,4-tetramethylcyclobutoxy)-3-chloropicolinonitrile dihydrochloride (D349)

To a stirred solution of tert-butyl ((1r,3r)-3-((5-chloro-6-cyanopyridin-3-yl)oxy)-2,2,4,4-tetramethylcyclobutyl)carbamate (may be prepared as described in Description 348; 2.88 g, 7.58 mmol) in DCM (75 mL) was added HCl (4M in dioxane, 19.0 mL, 75.8 mmol) at 0° C., then the mixture was stirred at room temperature for 18 hours. The reaction mixture was concentrated in vacuo to give the desired product (2.40 g, 85% yield) as a white solid. LCMS (formic A): Rt=0.76 min, MH⁺=280.

### Description 350

### N-((1r,3r)-3-((5-Chloro-6-cyanopyridin-3-yl)oxy)-2,2,4,4-tetramethylcyclobutyl)-6-(4-(hydroxymethyl)piperidin-1-yl)pyridazine-3-carboxamide (D350)

To a stirred solution of 5-((1r,3r)-3-amino-2,2,4,4-tetramethylcyclobutoxy)-3-chloropicolinonitrile, dihydrochloride (may be prepared as described in Description 349; 400 mg, 1.13 mmol) and 6-(4-(hydroxymethyl)piperidin-1-yl)pyridazine-3-carboxylic acid (may be prepared as described in Description 61, 323 mg, 1.36 mmol) in DMF (10 mL) was added HATU (647 mg, 1.70 mmol) and DIPEA (1.09 mL, 6.24 mmol) then the mixture stirred at room temperature for 18 hours. The mixture was quenched with NH₄Cl (1N), diluted with ethyl acetate, then the organic layer was separated. The organic layer was washed twice with NH₄Cl (1N), dried over Na₂SO₄, filtered and concentrated in vacuo. The sample was purified by chromatography on Si (12 g) using a 0-60% [3:1 ethyl acetate:ethanol]-cyclohexane gradient. The appropriate fractions were combined and evaporated in vacuo to give the desired product (358 mg, 63% yield) as a white solid. LCMS (formic A): Rt=1.26 min, MH⁺=500.

### Description 351

### N-((1r,3r)-3-((5-Chloro-6-cyanopyridin-3-yl)oxy)-2,2,4,4-tetramethylcyclobutyl)-2-(4-(hydroxymethyl)piperidin-1-yl)pyrimidine-5-carboxamide (D351)

To a stirred solution of 2-(4-(hydroxymethyl)piperidin-1-yl)pyrimidine-5-carboxylic acid (may be prepared as described in Description 63, 80.0 mg, 337 μmol), DIPEA (294 μL, 5 equivalents, 1.69 mmol) and 5-((1r,3r)-3-amino-2,2,4,4-tetramethylcyclobutoxy)-3-chloropicolinonitrile, dihydrochloride (may be prepared as described in Description 349, 125 mg, 354 μmol) in DMF (6.00 mL) was added HATU then the mixture was stirred (153.85 mg, 1.2 Eq, 404.62 μmol) was added and the resulting mixture was stirred at room temperature for 14.5 h. The mixture was treated with NH₄Cl (sat. aq., 10 mL) and extracted with ethyl acetate (2×30 mL). The organic layers were combined, washed sequentially with NH₄Cl (sat. aq., 10 mL) and brine (10 mL), dried over Na₂SO₄, filtered and concentrated in vacuo. The sample was purified by chromatography on Si (10 g) using a 0-100% ethyl acetate-cyclohexane gradient. The appropriate fractions were combined and evaporated in vacuo to give the desired product (164 mg, 93% yield) as a pale yellow solid. LCMS (formic A): Rt=1.28 min, MH⁺=499.

### Description 352

### N-((1r,3r)-3-((5-Chloro-6-cyanopyridin-3-yl)oxy)-2,2,4,4-tetramethylcyclobutyl)-6-(4-(hydroxymethyl)piperidin-1-yl)nicotinamide (D352)

To a stirred solution of 5-((1r,3r)-3-amino-2,2,4,4-tetramethylcyclobutoxy)-3-chloropicolinonitrile, dihydrochloride (may be prepared as described in Description 349, 985.21 mg, 1.1 equivalent, 2.7934 mmol) and 6-(4-(hydroxymethyl)piperidin-1-yl) nicotinic acid (may be prepared as described in Description 64, 600 mg, 2.54 mmol) in DMF (26 mL) was added HATU (1.26 g, 3.30 mmol) and DIPEA (2.21 mL, 12.7 mmol) then the mixture stirred at room temperature for 2 h. The mixture was quenched with NaHCO₃ (saturated aqueous), diluted with ethyl acetate, then the phases were separated. The organic layer was evaporated in vacuo and the resulting solid was triturated with methanol, filtered and rinsed with further methanol to give the desired product (1.10 g, 83% yield) a white solid. LCMS (formic A): Rt=1.10 min, MH⁺=498.

### Description 353

### N-((1r,3r)-3-((5-Chloro-6-cyanopyridin-3-yl)oxy)-2,2,4,4-tetramethylcyclobutyl)-6-(4-formylpiperidin-1-yl)nicotinamide (D353)

N-((1r,3r)-3-((5-Chloro-6-cyanopyridin-3-yl)oxy)-2,2,4,4-tetramethylcyclobutyl)-6-(4-(hydroxymethyl)piperidin-1-yl)nicotinamide (may be prepared as described in Description 352; 1.10 g, 2.21 mmol) was dissolved in DCM (50 mL) and cooled in an ice bath, then DMP (1.22 g, 2.87 mmol) was added, then the mixture was stirred at room temperature for 3 h. The reaction mixture was concentrated in vacuo, then directly purified by chromatography on Si (25 g) using a 0-100% ethyl acetate-cyclohexane gradient. The appropriate fractions were combined and evaporated in vacuo to give the desired product (911 mg, 79% yield) as a white solid. LCMS (formic A): Rt=1.20 min, MH⁺=496.

### Description 354

### N-((1r,3r)-3-((8-Cyanoquinolin-5-yl)oxy)-2,2,4,4-tetramethylcyclobutyl)-2-(4-(hydroxymethyl)piperidin-1-yl)picolinamide (D354)

To a mixture of 5-(4-(hydroxymethyl)piperidin-1-yl) picolinic acid hydrochloride (102 mg, 80% wt, 0.299 mmol), DIPEA (236 μL, 1.36 mmol) and 5-((1r,3r)-3-amino-2,2,4,4-tetramethylcyclobutoxy)quinoline-8-carbonitrile dihydrochloride (may be prepared as described in Description 343, 100 mg, 0.272 mmol) in DMF (3 mL) was added HATU (124 mg, 0.326 mmol) then the mixture was stirred at room temperature for 14.5 h. The mixture was treated with NH₄Cl (saturated aqueous, 10 mL) and extracted with ethyl acetate (2×30 mL). The organic layers were combined, washed sequentially with NH₄Cl (saturated aqueous, 10 mL) and brine (10 mL), was dried over Na₂SO₄, filtered and concentrated under reduced pressure. The sample was purified by chromatography on Si (10 g) using a 0-100% ethyl acetate-cyclohexane gradient, then the appropriate fractions were combined and evaporated in vacuo to give the desired product (108 mg, 70% yield) as a white solid. LCMS (formic A): Rt=1.26 min, MH⁺=514.

### Description 355

### N-((1r,3r)-3-((8-Cyanoquinolin-5-yl)oxy)-2,2,4,4-tetramethylcyclobutyl)-6-(4-(hydroxymethyl)piperidin-1-yl)nicotinamide (D355)

To a stirred solution of 6-(4-(hydroxymethyl)piperidin-1-yl) nicotinic acid (may be prepared as described in Description 64, 100 mg, 423 μmol), 5-((1r,3r)-3-amino-2,2,4,4-tetramethylcyclobutoxy)quinoline-8-carbonitrile, dihydrochloride (may be prepared as described in Description 343, 187 mg, 508 μmol), and DIPEA (274 mg, 0.369 mL, 5.01 equivalents, 2.12 mmol) in DMF (4.0 mL) was added HATU (193 mg, 508 μmol) then the mixture was stirred at room temperature for 15 h. The reaction mixture was quenched with NaHCO₃ (saturated aqueous) and with ethyl acetate three times. The combined organic layers were dried over anhydrous Na₂SO₄, filtered and concentrated in vacuo. The sample was purified by chromatography on Si (40 g) using a 0-100% ethyl acetate-cyclohexane over 16 column volumes. The appropriate fractions were combined and evaporated in vacuo to give the desired product (238 mg, quantitative yield) as a white solid. LCMS (high pH B): Rt=3.12 min, MH⁺=514.

### Description 356

### tert-Butyl(S)-2-(((methylsulfonyl)oxy)methyl) morpholine-4-carboxylate (D356)

To a solution of tert-butyl(S)-2-(hydroxymethyl) morpholine-4-carboxylate (8.50 g, 39.1 mmol) in Dichloromethane (DCM) (100 mL) stirred under nitrogen at −30° C. was added TEA (16.4 mL, 117 mmol) followed by Ms-Cl (3.66 mL, 46.9 mmol). The reaction mixture was stirred at −30° C. for 30 min. The reaction mixture was quenched with ice water (500 mL) and extracted with DCM (2×500 mL), then the organic layers were washed with brine, dried over anhydrous sodium sulfate, filtered and concentrated under reduced pressure to give the desired product (11.5 g, 38.9 mmol, 99% yield) as pale yellow gum. LCMS (formic A): Rt=0.88 min, M-Boc+H⁺=196.

### Description 357

### tert-Butyl (R)-2-((4-bromo-1H-indol-1-yl)methyl) morpholine-4-carboxylate (D357)

To a solution of 4-bromo-1H-indole (5.08 g, 25.9 mmol) and potassium tert-butoxide (7.27 g, 64.8 mmol) in DMF (150 mL) stirred under nitrogen at room temperature was added tert-butyl(S)-2-(((methylsulfonyl)oxy)methyl) morpholine-4-carboxylate (may be prepared as described in Description 356; 11.5 g, 38.9 mmol) followed by molecular sieves. The reaction mixture was stirred at 60° C. for 16 h. The reaction mixture was quenched with ice water (300 mL) and extracted with ethyl acetate (2×300 mL) and washed with brine. The combined organic layers were dried over anhydrous sodium sulfate, filtered and concentrated under reduced pressure. This procedure was repeated (using 25.5 mmol 4-bromo-1H-indole), then the two batches were combined, loaded on silica and purified by chromatography on silica (330 g) using a 0-20% ethyl acetate-cyclohexane gradient. The appropriate fractions were combined and evaporated in vacuo to give the desired product (12.5 g, 51% yield based on combined batches) as a colourless liquid. LCMS (formic A): Rt=1.34 min, M-^{t}Bu+H⁺=399, 341.

### Description 358

### tert-Butyl (R)-2-((4-(2,4-dioxo-3-((2-(trimethylsilyl)ethoxy)methyl)tetrahydropyrimidin-1(2H)-yl)-1H-indol-1-yl)methyl) morpholine-4-carboxylate (D358)

To a stirred solution of tert-butyl (R)-2-((4-bromo-1H-indol-1-yl)methyl) morpholine-4-carboxylate (may be prepared as described in Description 357; 5.00 g, 12.7 mmol) in 1,4-dioxane (50 mL) was added potassium carbonate (5.24 g, 37.9 mmol) followed by 3-((2-(trimethylsilyl)ethoxy)methyl)dihydropyrimidine-2,4(1H,3H)-dione (may be prepared as described in Description 1; 3.40 g, 13.91 mmol) and molecular sieves (1.0 g) under nitrogen purging followed by added CuI (0.361 g, 1.897 mmol) under nitrogen atmosphere at room temperature and degassed for 5 min. Then (1R,2R)—N1,N2-dimethylcyclohexane-1,2-diamine (0.540 g, 3.79 mmol) was added and the mixture was heated at 110° C. in sealed tube for 16 h. The reaction mixture was quenched with ice water (300 mL) and extracted with ethyl acetate (2×300 mL). The combined organic layers were washed with brine, dried over anhydrous sodium sulfate, filtered and concentrated under reduced pressure. This procedure was repeated (using 12.7 mmol tert-butyl (R)-2-((4-bromo-1H-indol-1-yl)methyl) morpholine-4-carboxylate), then the two batches were combined, loaded on silica and purified by chromatography on silica (330 g) using a 0-50% ethyl acetate-cyclohexane gradient. The appropriate fractions were combined and evaporated in vacuo to give the desired product (9.9 g, 70% yield based on combined batches) as a pale yellow gum. LCMS (formic A): Rt=1.31 min, M-^{t}Bu+H⁺=503.

### Description 359

### tert-Butyl (R)-2-(((methylsulfonyl)oxy)methyl) morpholine-4-carboxylate (D359)

To a solution of tert-butyl (R)-2-(hydroxymethyl) morpholine-4-carboxylate (1.10 g, 5.06 mmol) in DCM (5.0 mL) stirred under nitrogen at −30° C. was added TEA (2.112 mL, 15.2 mmol) followed by Ms-Cl (0.473 mL, 6.08 mmol), then the mixture was stirred at −30° C. for 20 min. The reaction mixture was quenched with ice water (50 mL) and extracted with DCM (2×50 mL). The organic layers were combined, washed with brine, dried over anhydrous sodium sulfate, filtered and concentrated under reduced pressure to give the desired product (1.50 g, 99% yield) as a colourless gum. LCMS (formic A): Rt=0.76 min, M-Boc+H⁺=196.

### Description 360

### tert-Butyl (S)-2-((4-bromo-1H-indol-1-yl)methyl) morpholine-4-carboxylate (D360)

To a stirred solution of 4-bromo-1H-indole (0.65 g, 3.32 mmol) and cesium carbonate (3.24 g, 9.95 mmol) in DMF (15 mL) was added tert-butyl (R)-2-(((methylsulfonyl)oxy)methyl) morpholine-4-carboxylate (may be prepared as described in Description 359; 1.47 g, 4.97 mmol). The reaction mixture was stirred at 60° C. for 16 h. The reaction mixture was quenched with water (70 mL) and extracted with ethyl acetate (2×50 mL). The organic layer were combined, washed with brine, dried over sodium sulfate, filtered and concentrated under reduced pressure. The sample was and absorbed onto silica and purified by chromatography on silica (25 g) using a 0-10% ethyl acetate-cyclohexane gradient. The appropriate fractions were combined and evaporated in vacuo to give the desired product (900 mg, 68% yield) as a pale yellow liquid. LCMS (formic A): Rt=1.33 min, M-^{t}Bu+H⁺=399, 341.

### Description 361

### tert-Butyl(S)-2-((4-(2,4-dioxo-3-((2-(trimethylsilyl)ethoxy)methyl)tetrahydropyrimidin-1(2H)-yl)-1H-indol-1-yl)methyl) morpholine-4-carboxylate (D361)

To a stirred solution of tert-butyl(S)-2-((4-bromo-1H-indol-1-yl)methyl) morpholine-4-carboxylate (may be prepared as described in Description 360; 1.5 g, 3.79 mmol) in DMF (15 mL) was added potassium carbonate (1.57 g, 11.4 mmol) followed by 3-((2-(trimethylsilyl)ethoxy)methyl)dihydropyrimidine-2,4(1H,3H)-dione (may be prepared as described in Description 1; 1.02 g, 4.17 mmol) and was added molecular sieves (500 mg) under nitrogen purging followed by CuI (0.108 g, 0.569 mmol), then (1R,2R)—N1,N2-dimethylcyclohexane-1,2-diamine (0.162 g, 1.14 mmol) was added and the mixture was heated at 110° C. for for 32 h. The reaction mixture was quenched with ice water (100 mL) and extracted with ethyl acetate (2×100 mL). The organic layers were combined, washed with brine, dried over sodium sulfate, filtered and concentrated under reduced pressure. This procedure was repeated (using 3.79 mmol tert-butyl(S)-2-((4-bromo-1H-indol-1-yl)methyl) morpholine-4-carboxylate), then the two batches were combined, loaded on silica and purified by chromatography on silica (110 g) using a 0-50% ethyl acetate-cyclohexane gradient. The appropriate fractions were combined and evaporated in vacuo to give the desired product (2.60 g, 59% yield based on combined batches) as a pale yellow gum. LCMS (formic A): Rt=1.30 min, M+Na+H⁺=581.

### Description 362

### N-((1r,3r)-3-((8-Cyanoquinolin-5-yl)oxy)-2,2,4,4-tetramethylcyclobutyl)-6-(4-formylpiperidin-1-yl)nicotinamide (D362)

DMP (297 mg, 701 μmol) was added to a stirred solution of N-((1r,3r)-3-((8-cyanoquinolin-5-yl)oxy)-2,2,4,4-tetramethylcyclobutyl)-6-(4-(hydroxymethyl)piperidin-1-yl)nicotinamide (may be prepared as described in Description 355, 300 mg, 584 μmol) in DCM (6 mL), then the mixture was stirred for 3 h. The mixture was diluted with ethyl acetate, NaHCO₃ (saturated aqueous) and Na₂CO₃ (10% aq.) then the phases were separated. The aqueous layer as back-extracted with ethyl acetate, then the organic layers were combined, dried over anhydrous sodium sulfate, filtered and concentrated in vacuo. The sample was purified by chromatography on silica (10 g) using a 0-100% ethyl acetate-cyclohexane gradient. The appropriate fractions were combined and evaporated in vacuo to give the desired product (238 mg, 80% yield) as a yellow foam. LCMS (formic A): Rt=1.16 min, MH⁺=512.

### Description 363

### Methyl 5-(4-(hydroxymethyl)piperidin-1-yl) picolinate (D363)

To a solution of methyl 5-fluoropyridine-2-carboxylate (335 mg, 2.16 mmol) in DMSO (1 mL) were added K₂CO₃ (358 mg, 2.59 mmol) and 4-piperidinemethanol (298 mg, 2.59 mmol). The resulting mixture was stirred at 110° C. for 75 min. The mixture was diluted with water, then extracted with DCM (3×25 ml). The combined organic phases were dried over Na₂SO₄, filtered and evaporated in vacuo to give the desired product (479 mg, 84% yield) as a white solid. LCMS (high pH B): Rt=1.76 min, MH⁺=251.

### Description 364

### 5-(4-(Hydroxymethyl)piperidin-1-yl) picolinic acid hydrochloride (D364)

To a stirred suspension of methyl 5-(4-(hydroxymethyl)piperidin-1-yl) picolinate (may be prepared as described in Description 363; 430 mg, 1.72 mmol) in THF (4 mL) was added NaOH (2M aq., 1.00 mL, 2.00 mmol) and the mixture stirred for 22.5 h, after which time a precipitate had formed. The precipitate was separated by filtration and washed with THF. The solid was dissolved in water (10 mL) and the pH adjusted to 0 with HCl (2M aqueous). The reaction mixture was concentrated in vacuo to, then the sample was then azeotroped with toluene (3×25 mL) and with diethyl ether (3×10 ml) then dried under vacuum to give the desired product (539 mg, 80% w/w, 94% yield) as a yellow solid. LCMS (high pH B): Rt=1.68 min, MH⁺=237.

### Description 365

### N-((1r,3r)-3-((8-Cyanoquinolin-5-yl)oxy)-2,2,4,4-tetramethylcyclobutyl)-5-(4-(hydroxymethyl)piperidin-1-yl)picolinamide (D365)

To a mixture of 5-(4-(hydroxymethyl)piperidin-1-yl) picolinic acid hydrochloride (may be prepared as described in Description 364; 102 mg, 80% w/w, 0.299 mmol), DIPEA (236 μL, 1.36 mmol) and 5-((1r,3r)-3-amino-2,2,4,4-tetramethylcyclobutoxy)quinoline-8-carbonitrile dihydrochloride (may be prepared as described in Description 343, 100 mg, 0.272 mmol) in DMF (3 mL) was added HATU (124 mg, 0.326 mmol) then the mixture was stirred at room temperature for 14.5 h. The mixture was treated with NH₄Cl (saturated aqueous, 10 mL) and extracted with ethyl acetate (2×30 mL). The organic layers were combined, washed sequentially with NH₄Cl (saturated aqueous, 10 mL) and brine (10 mL), was dried over Na₂SO₄, filtered, and concentrated under reduced pressure. The sample was purified by chromatography on Si (10 g) using a 0-100% ethyl acetate-cyclohexane gradient, then the appropriate fractions were combined and evaporated in vacuo to give the desired product (108 mg, 70% yield) as a white solid. LCMS (formic A): Rt=1.26 min, MH⁺=514.

### Description 366

### Methyl 5-(4-(hydroxymethyl)piperidin-1-yl)pyrimidine-2-carboxylate (D366)

To a solution of methyl 5-chloropyrimidine-2-carboxylate (1.00 g, 5.79 mmol), 4-piperidinemethanol (801 mg, 6.95 mmol) in DMSO (12 mL) was added DIPEA (5.05 mL, 29.0 mmol) and heated in the microwave at 120° C. for 3 h. The reaction mixture was concentrated in vacuo, then purified by preparative HPLC (XBridge 50 mm×100 mm) using a 0-100% acetonitrile-water (+0.1% ammonium bicarbonate modifier adjusted to pH 10 with aqueous ammonia) gradient over 15 min (flow rate 40 mL/min), then the appropriate fractions were combined and evaporated in vacuo to give the desired product (697 mg, 45% yield) as a white solid. LCMS (formic A): Rt=0.66 min, MH⁺=252.

### Description 367

### 5-(4-(Hydroxymethyl)piperidin-1-yl)pyrimidine-2-carboxylic acid (D367)

To methyl 5-(4-(hydroxymethyl)piperidin-1-yl)pyrimidine-2-carboxylate (may be prepared as described in Description 366; 943.00 mg, 1 equivalent, 3.75 mmol) in methanol (7.5 mL) was added NaOH (2M aq., 2.06 mL, 4.13 mmol) then the mixture was stirred at 60° C. for 1 h. The mixture was cooled to room temperature, then the resulting precipitate was separated. The solid was directly dissolved in HCl (2M aq.) until pH=0, then the mixture was concentrated in vacuo, azeotroped with toluene (3×30 ml) and diethyl ether (2×15 mL), then dried under vacuum to give the desired product (1.06 g, 80% w/w, 95% yield) as a yellow solid. LCMS (formic A): Rt=0.41 min, MH⁺=238.

### Description 368

### N-((1r,3r)-3-((5-Chloro-6-cyanopyridin-3-yl)oxy)-2,2,4,4-tetramethylcyclobutyl)-5-(4-(hydroxymethyl)piperidin-1-yl)pyrimidine-2-carboxamide (D368)

To a stirred solution of 5-(4-(hydroxymethyl)piperidin-1-yl)pyrimidine-2-carboxylic acid (may be prepared as described in Description 367; 252 mg, 56% weight, 595 μmol) in DMF (2.1 mL) was added HATU (243 mg, 638 μmol) and 5-((1r,3r)-3-amino-2,2,4,4-tetramethylcyclobutoxy)-3-chloropicolinonitrile dihydrochloride (may be prepared as described in Description 349, 150 mg, 425 μmol). DIPEA (482 μL, 2.76 mmol) was added then the mixture stirred at room temperature for 2.5 h. The mixture was diluted with ethyl acetate and NH₄Cl (saturated aqueous) then the phases were separated. The aqueous phase extracted with ethyl acetate three times, then the organic phases were combined, washed with brine, dried over Na₂SO₄, filtered and the solvent removed under reduced pressure. The was purified by flash column chromatography on silica (12 g) using a 0-100% [ethyl acetate:ethanol (3:1)]-cyclohexane gradient, then the appropriate fractions were combined and evaporated in vacuo to give the desired product (173 mg, 74% yield) as a white solid. LCMS (high pH B): Rt=2.92 min, MH⁺=499.

### Description 369

### N-((1r,3r)-3-((5-Chloro-6-cyanopyridin-3-yl)oxy)-2,2,4,4-tetramethylcyclobutyl)-5-(4-formylpiperidin-1-yl)pyrimidine-2-carboxamide (D369)

To a stirred solution of N-((1r,3r)-3-((5-chloro-6-cyanopyridin-3-yl)oxy)-2,2,4,4-tetramethylcyclobutyl)-5-(4-(hydroxymethyl)piperidin-1-yl)pyrimidine-2-carboxamide (may be prepared as described in Description 368; 277 mg, 555 μmol) in DCM (7 mL) at 0° C. was added DMP (283 mg, 666 μmol) then the mixture was stirred at room temperature for 75 min. The mixture was diluted with DCM and washed sequentially with NaHCO₃ (saturated aqueous) and Na₂CO₃ (10% aq.). The combined aqueous phases were back-extracted with DCM twice, then the organic layers were combined, dried over Na₂SO₄, filtered and the solvent removed under reduced pressure. The sample was purified by chromatography on silica (25 g) using a 0-100% [ethyl acetate:ethanol (3:1)]-cyclohexane gradient, then the appropriate fractions were combined and evaporated in vacuo to give the desired product (158 mg, 51% yield) as a yellow foam. LCMS (high pH B): Rt=2.20 min, MH+=497.

### Description 370

### tert-Butyl ((1r,3r)-3-((6-cyano-5-(trifluoromethyl)pyridin-3-yl)oxy)-2,2,4,4-tetramethylcyclobutyl)carbamate (D370)

A stirred solution of tert-butyl ((1r,3r)-3-hydroxy-2,2,4,4-tetramethylcyclobutyl)carbamate (500 mg, 2.05 mmol) in THF (4 mL), was cooled to 0° C., then NaH (107 mg, 60% w/w, 2.67 mmol) was added and the reaction mixture was stirred at room temperature for 10 min, then 5-bromo-3-(trifluoromethyl)pyridine-2-carbonitrile (567 mg, 1.1 equivalent, 2.26 mmol) was added portion-wise and the mixture stirred at room temperature for 18 h. The mixture was quenched with NH₄Cl (sat. aq.) and the solution extracted with ethyl acetate (3×20 mL). The organic layers were combined, dried over Na₂SO₄, filtered and the solvent removed under reduced pressure. The sample was purified by chromatography on silica (10 g) using a 0-15% ethyl acetate-cyclohexane gradient over 16 column volumes. The appropriate fractions were combined and evaporated in vacuo to give the desired product (491 mg, 55% yield) as a white solid. LCMS (high pH B): Rt=3.04 min, MH⁺=does not ionise.

### Description 371

### 5-((1r,3r)-3-Amino-2,2,4,4-tetramethylcyclobutoxy)-3-(trifluoromethyl) picolinonitrile dihydrochloride (D371)

To a stirred solution of tert-butyl ((1r,3r)-3-((6-cyano-5-(trifluoromethyl)pyridin-3-yl)oxy)-2,2,4,4-tetramethylcyclobutyl)carbamate (may be prepared as described in Description 370; 487 mg, 1.18 mmol) in DCM (11 mL) was added HCl (4M in dioxane, 2.95 mL, 11.8 mmol) at 0° C., then the mixture was stirred at room temperature for 21 h. The reaction mixture was concentrated in vacuo and dried to give the desired product (411 mg, 86% yield) as a white solid. LCMS (formic A): Rt=0.83 min, MH⁺=314.

### Description 372

### N-((1r,3r)-3-((6-Cyano-5-(trifluoromethyl)pyridin-3-yl)oxy)-2,2,4,4-tetramethylcyclobutyl)-5-(4-(hydroxymethyl)piperidin-1-yl)pyrimidine-2-carboxamide (D372)

To a solution of 5-(4-(hydroxymethyl)piperidin-1-yl)pyrimidine-2-carboxylic acid (may be prepared as described in Description 367; 345 mg, 56% w/w, 816 μmol and 92 mg, 80% w/w, 311 μmol) in DMF (3.9 mL) was added 5-((1r,3r)-3-amino-2,2,4,4-tetramethylcyclobutoxy)-3-(trifluoromethyl) picolinonitrile dihydrochloride (may be prepared as described in Description 371; 300 mg, 777 μmol) and HATU (443 mg, 1.17 mmol), followed by DIPEA (879 μL, 5.05 mmol) then the mixture was stirred at room temperature for 90 min. The mixture diluted with ethyl acetate and NH₄Cl (saturated aqueous), then the phases were separated. The aqueous layer was back-extracted three times with ethyl acetate, then the organic layers were combined, washed with brine, dried over Na₂SO₄, filtered, and evaporated in vacuo. The sample was purified by chromatography on silica (25 g) using a 0-100% [ethyl acetate:ethanol (3:1)]-cyclohexane gradient, then the appropriate fractions were combined and evaporated in vacuo. The sample was dissolved in DCM, washed with water and the concentrated in vacuo to give the desired product (324 mg, 71% yield) as a white solid. LCMS (high pH B): Rt=2.14 min, MH⁺=533.

### Description 373

### N-((1r,3r)-3-((6-Cyano-5-(trifluoromethyl)pyridin-3-yl)oxy)-2,2,4,4-tetramethylcyclobutyl)-5-(4-formylpiperidin-1-yl)pyrimidine-2-carboxamide (D373)

To a stirred solution of N-((1r,3r)-3-((6-cyano-5-(trifluoromethyl)pyridin-3-yl)oxy)-2,2,4,4-tetramethylcyclobutyl)-5-(4-(hydroxymethyl)piperidin-1-yl)pyrimidine-2-carboxamide (may be prepared as described in Description 372; 312 mg, 586 μmol) in DCM (7.5 mL) at 0° C. was added DMP (298 mg, 703 μmol) then the mixture was stirred at room temperature for 75 min. The mixture was diluted with DCM and washed sequentially with NaHCO₃ (saturated aqueous) and Na₂CO₃ (10% aqueous). The combined aqueous phases were back-extracted with DCM twice, then the organic layers were combined, dried over Na₂SO₄, filtered and the solvent removed under reduced pressure. The sample was purified by chromatography on silica (25 g) using a 0-100% [ethyl acetate:ethanol (3:1)]-cyclohexane gradient, then the appropriate fractions were combined and evaporated in vacuo to give the desired product (228 mg, 67% yield) as a yellow foam. LCMS (high pH B): Rt=2.25 min, MH⁺=531.

### Description 374

### N-((1r,3r)-3-(3-Chloro-4-cyanophenoxy)-2,2,4,4-tetramethylcyclobutyl)-5-(4-(hydroxymethyl)piperidin-1-yl)pyrimidine-2-carboxamide (D374)

To a solution of 5-(4-(hydroxymethyl)piperidin-1-yl)pyrimidine-2-carboxylic acid (may be prepared as described in Description 367; 415 mg, 80% weight, 1.3 equivalents, 1.40 mmol) in DMF (5.4 mL), was added 4-((1r,3r)-3-amino-2,2,4,4-tetramethylcyclobutoxy)-2-chlorobenzonitrile (may be prepared as described in Description 49, 300 mg, 1.0761 mmol) and HATU (614 mg, 1.61 mmol), followed by DIPEA (656 μL, 3.77 mmol) then the mixture was stirred at room temperature for 3 h. The mixture was diluted with ethyl acetate and NH₄Cl (saturated aqueous) then the phases were separated. The aqueous layer was back-extracted with ethyl acetate three times, then the organic layers were combined, washed with brine, dried over Na₂SO₄, filtered and concentrated in vacuo. The sample was purified by chromatography on silica (25 g) using a 0-100% [ethyl acetate:ethanol (3:1)]-cyclohexane gradient, then the appropriate fractions were combined and evaporated in vacuo to give the desired product (316 mg, 51% yield) as a pale yellow foam. LCMS (high pH B): Rt=2.18 min, MH⁺=498.

### Description 375

### N-((1r,3r)-3-(3-Chloro-4-cyanophenoxy)-2,2,4,4-tetramethylcyclobutyl)-5-(4-formylpiperidin-1-yl)pyrimidine-2-carboxamide (D375)

To a stirred solution of N-((1r,3r)-3-(3-chloro-4-cyanophenoxy)-2,2,4,4-tetramethylcyclobutyl)-5-(4-(hydroxymethyl)piperidin-1-yl)pyrimidine-2-carboxamide (may be prepared as described in Description 374; 312 mg, 570 μmol) in DCM (7.2 mL) was added DMP (290 mg, 684 μmol) then the mixture was stirred at room temperature for 75 min. The mixture was diluted with DCM and washed sequentially with NaHCO₃ (saturated aqueous) and Na₂CO₃ (10% aqueous.). The combined aqueous phases were back-extracted with DCM twice, then the organic layers were combined, dried over Na₂SO₄, filtered and the solvent removed under reduced pressure. The sample was purified by chromatography on silica (25 g) using a 0-100% [ethyl acetate:ethanol (3:1)]-cyclohexane gradient, then the appropriate fractions were combined and evaporated in vacuo to give the desired product (127 mg, 40% yield) as a yellow foam. LCMS (high pH B): Rt=2.30 min, MH⁺=496.

### (Cereblon Binder) Degron Examples

The following compounds are compounds of formula (I), (Ia) or (Ib) where p and q are both 0. They also fall into the scope of formulae (Iaa) or (Ibb).

### Example CB1

### 1-(1-(Piperidin-4-yl)-1H-indol-4-yl)dihydropyrimidine-2,4(1H,3H)-dione (ECB1)

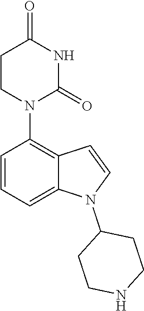 [SMILES: O=C1CCN(c2cccc3c2ccn3C2CCNCC2)C(=O)N1]

### Batch 1

Benzyl 4-(4-(2,4-dioxotetrahydropyrimidin-1(2H)-yl)-1H-indol-1-yl)piperidine-1-carboxylate (may be prepared as described in Description 5; 2.75 g, 6.16 mmol) was dissolved in a mixture of MeOH (50 mL) and EtOAc (50.0 mL), then ammonium formate (0.388 g, 6.16 mmol) and Pd—C 10% on Carbon (0.655 g, 6.16 mmol) were added and the mixture was heated at reflux for 1 h. The mixture was cooled and filtered through Celite, the Celite pad washed with 1:1 DCM/MeOH (50 mL) and the filtrate evaporated in vacuo to give Batch 1 of the title compound (1.67 g).

### Batch 2

Benzyl 4-(4-(2,4-dioxotetrahydropyrimidin-1(2H)-yl)-1H-indol-1-yl)piperidine-1-carboxylate (may be prepared as described in Description 5; 5.2 g, 11.65 mmol) was dissolved in a mixture of MeOH (200 mL) and EtOAc (50 mL) and Pd—C 10% on Carbon (1.239 g, 11.65 mmol) and ammonium formate (3.67 g, 58.2 mmol) were added, then the mixture was heated at reflux for 30 min, cooled and filtered through Celite. The Celite pad was washed with 1:1 DCM/MeOH (50 mL) and the filtrate evaporated in vacuo to give Batch 2 of the title compound (3.35 g). the two batches were combined and triturated with MTBE (30 mL), then filtered to give the title compound as a colourless solid (4.48 g, 14.34 mmol, 80% overall yield).

### Example CB2

### 1-(1-(Piperidin-4-ylmethyl)-1H-indol-4-yl)dihydropyrimidine-2,4(1H,3H)-dione (ECB2)

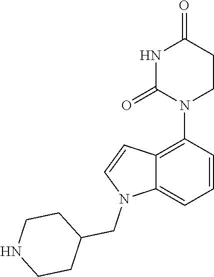 [SMILES: O=C1CCN(c2cccc3c2ccn3CC2CCNCC2)C(=O)N1]

To tert-butyl 4-((4-(2,4-dioxo-3-((2-(trimethylsilyl)ethoxy)methyl)tetrahydropyrimidin-1(2H)-yl)-1H-indol-1-yl)methyl)piperidine-1-carboxylate (may be prepared as described in Description 7; 528 mg, 0.948 mmol) was added TFA (1.6 mL, 20.77 mmol), and the reaction mixture was stirred at room temperature for 1 h. The solvent was removed in vacuo, transferred to a vial by dissolution in DCM and the solvent was removed under nitrogen blowdown. The crude material was dissolved in 1:1 DMSO:MeOH (3 mL) and purified by MDAP (high pH) in 3 portions. Fractions containing the desired product were collected, and the solvent was removed in vacuo to give the title compound as a light brown foam (167 mg, 0.384 mmol, 41% yield). LCMS (high pH A): Rt=0.64 min, MH⁺=327.

### Example CB3

### 1-(1-(3,3-Difluoropiperidin-4-yl)-1H-indol-4-yl)dihydropyrimidine-2,4(1H,3H)-dione (ECB3)

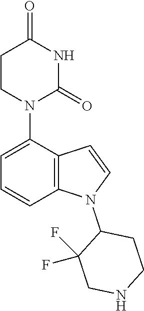 [SMILES: O=C1CCN(c2cccc3c2ccn3C2CCNCC2(F)F)C(=O)N1]

3-((2-(Trimethylsilyl)ethoxy)methyl)dihydropyrimidine-2,4(1H,3H)-dione (maybe prepared as described+ in Description 1; 0.647 g, 2.65 mmol), tert-butyl 4-(4-bromo-1H-indol-1-yl)-3,3-difluoropiperidine-1-carboxylate (may be prepared as described in Description 8; 1 g, 2.408 mmol), potassium carbonate (0.666 g, 4.82 mmol), trans-N,N′-Dimethylcyclohexane-1,2-diamine (0.114 mL, 0.722 mmol) and copper(I) iodide (0.069 g, 0.361 mmol) in anhydrous 1,4-dioxane (10 mL) were mixed, degassed with vacuum/N₂ several times, then heated at 135° C. in a sealed tube overnight (ca 19 h). The reaction was cooled, diluted with water, extracted with EtOAc, washed with brine twice, dried, filtered and evaporated in vacuo to give a pale brown oil. This was taken up in DCM (10.00 mL), treated with TFA (10 mL, 130 mmol) and stirred for 1 h, blown down, taken up in 4 M NH₃-MeOH, stirred 1 h, and blown down to give a crude solid. This material was diluted with ether, filtered, washed with ether and water and ether again, then dried in vacuo at 40° C. for 50 h. The material was flash chromatographed (C18, 150 g, 10-30% pH10 NH₄CO₂H aq, MeCN) to give the title compound as a white collapsed foam (220 mg, 0.600 mmol, 25% yield). LCMS (high pH A): Rt=0.66 min, MH⁺=349.

### Example CB4

### 1-(1-(Piperidin-4-yl)-1H-indazol-4-yl)dihydropyrimidine-2,4(1H,3H)-dione 2,2,2-trifluoroacetate (ECB4)

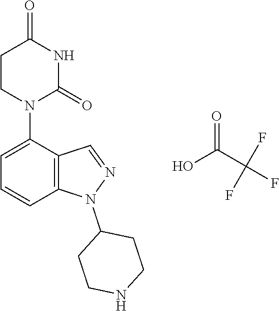 [SMILES: O=C1CCN(c2cccc3c2cnn3C2CCNCC2)C(=O)N1 ; O=C(O)C(F)(F)F]

### Batch 1

Tert-butyl 4-(4-(2,4-dioxo-3-((2-(trimethylsilyl)ethoxy)methyl)tetrahydropyrimidin-1(2H)-yl)-1H-indazol-1-yl)piperidine-1-carboxylate (may be prepared as described in Description 13; 12 g, 22.07 mmol) was dissolved in DCM (15.00 mL). TFA (15 mL) was added and the reaction mixture was stirred at room temperature for 16 h. The reaction mass was evaporated under reduced pressure. The material was dissolved in MeOH (50 mL) and then slowly added to 4 M ammonia in MeOH (30 mL) and stirred for 60 min. A white precipitate formed which was concentrated to afford an off-white solid. The solid was dissolved in hot EtOAc (200 mL) and washed with water (100 mL), brine (100 mL), dried over sodium sulphate and concentrated under reduced pressure to afford a solid.

### Batch 2

Tert-butyl 4-(4-(2,4-dioxo-3-((2-(trimethylsilyl)ethoxy)methyl)tetrahydropyrimidin-1(2H)-yl)-1H-indazol-1-yl)piperidine-1-carboxylate (may be prepared as described in Description 13; 13 g, 23.91 mmol) was dissolved in DCM (20.00 mL). TFA (20 mL) was added and the reaction mixture was stirred at room temperature for 16 h. The reaction mass was evaporated under reduced pressure. The material was dissolved in MeOH (50 mL) and then slowly added to 4 M ammonia in MeOH (30 mL) and stirred for 60 min. A white precipitate formed which was concentrated to afford an off-white solid. The solid was dissolved in hot EtOAc (200 mL) and washed with water (100 mL), brine (100 mL), dried over sodium sulphate and concentrated under reduced pressure to afford off a white solid.

Batch 1 and Batch 2 were combined and stirred in MeOH (150 mL) for 3 h. This was filtered, washed with water (2×100 mL) and dried to give the title compound (10.5 g, 24.52 mmol) as an off white solid. LCMS (formic A): Rt=0.28 min MH⁺=314

### Example CB5

### 1-(6-Chloro-1-(piperidin-4-yl)-1H-indazol-4-yl)dihydropyrimidine-2,4(1H,3H)-dione trifluoroacetate (ECB5)

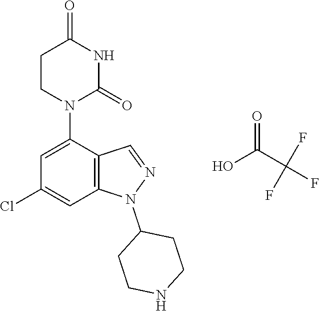 [SMILES: O=C1CCN(c2cc(Cl)cc3c2cnn3C2CCNCC2)C(=O)N1 ; O=C(O)C(F)(F)F]

A solution of tert-butyl 4-(6-chloro-4-(2,4-dioxo-3-((2-(trimethylsilyl)ethoxy)methyl)tetrahydropyrimidin-1(2H)-yl)-1H-indazol-1-yl)piperidine-1-carboxylate (may be prepared as described in Description 15; 131 mg, 0.227 mmol) in DCM (1 mL) was treated with TFA (0.250 mL, 3.24 mmol) and left to stand in a stoppered vessel for 4 h. The mixture was evaporated under a stream of nitrogen and the residue dissolved in MeOH (1 mL) and treated with 4 M ammonia in MeOH (1.699 mL, 6.80 mmol). The solution was stirred in a stoppered vessel at room temperature for 1 h and evaporated under a stream of nitrogen. The solid was triturated with EtOAc (2×2 mL) and dried in a vacuum oven to give the title compound as a white solid (108 mg, 0.234 mmol) which was used without purification in the next step. LCMS (high pH A): Rt=0.70 min, 347, 349 MH⁺=.

### Example CB6

### 1-(1-(Piperidin-4-yl)-1H-pyrrolo[2,3-c]pyridin-4-yl)dihydropyrimidine-2,4(1H,3H)-dione (ECB6)

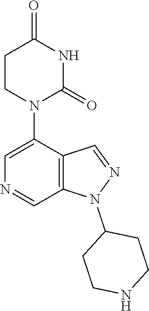 [SMILES: O=C1CCN(c2cncc3c2cnn3C2CCNCC2)C(=O)N1]

Potassium carbonate (5.09 g, 36.8 mmol), copper(I) iodide (0.526 g, 2.76 mmol), trans-N,N′-dimethylcyclohexane-1,2-diamine (0.871 mL, 5.52 mmol) 3-((2-(trimethylsilyl)ethoxy)methyl)dihydropyrimidine-2,4(1H,3H)-dione (may be prepared as described in Description 1; 4.95 g, 20.25 mmol) tert-butyl 4-(4-bromo-1H-pyrrolo[2,3-c]pyridin-1-yl)piperidine-1-carboxylate (may be prepared as described in Description 20; 7 g, 18.41 mmol) anhydrous 1,4-dioxane (150 mL) were degassed with N₂ for 10 min, then heated at 120° C. for 28 h, then allowed to cool to room temperature for 2 days. The reaction mixture was decanted, washing the sludge with more dioxane through Celite, and evaporated in vacuo. This was taken up in EtOAc and washed with water, dried (MgSO₄), filtered and evaporated in vacuo. Flash chromatography on 330 g silica eluting with 0-5% MeOH in DCM gave a green collapsed foam (5.2 g). This was taken up in DCM (10 mL) and treated with TFA (30 mL, 389 mmol) and stirred for 1 h, then blown down and held under vacuum. The reaction mixture was treated with 4 M NH₃ in MeOH and stirred. The reaction mixture was blown down and taken up in water. Flash chromatography was carried out on 150 g C18 media eluting with 0-25% MeCN in 10 mM aqueous NH₄CO₂H) in two batches. Evaporation of the fractions and drying in vacuo at 40° C. overnight gave the title compound as a white powder (2.2 g, 6.67 mmol, 36% yield). LCMS (high pH A): Rt=0.51 min, MH⁺=314

### Example CB7

### 1-(1-(Piperidin-4-yl)-1H-pyrazolo[4,3-c]pyridin-4-yl)dihydropyrimidine-2,4(1H,3H)-dione (ECB7)

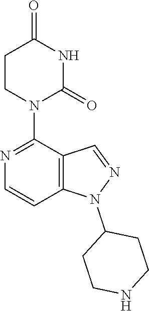 [SMILES: O=C1CCN(c2nccc3c2cnn3C2CCNCC2)C(=O)N1]

Benzyl 4-(4-(2,4-dioxo-3-((2-(trimethylsilyl)ethoxy)methyl)tetrahydropyrimidin-1(2H)-yl)-1H-pyrazolo[4,3-c]pyridin-1-yl)piperidine-1-carboxylate (may be prepared as described in Description 21; 600 mg, 1.037 mmol), Pd—C (10% w/w) (110 mg, 1.037 mmol), ammonium formate (654 mg, 10.37 mmol) were dissolved in ethanol (10 mL) and heated at 90° C. for 1 h. The reaction mixture was filtered, evaporated in vacuo, taken up in DCM (1 mL), treated with TFA (2 mL), stirred 1 h, concentrated under a stream of nitrogen, taken up in 4 M NH₃-MeOH and stirred 1 h. The reaction mixture was concentrated under a stream of nitrogen. Purification by flash column chromatography (C18-silica, 100 g, 10-25% 10 mM pH 10 NH₄CO₂H/aq-MeCN) gave the title compound as a white powder (270 mg, 0.816 mmol, 79% yield). LCMS (high pH A): Rt=0.51 min, MH⁺=315.1.

### Example CB8

### 1-(1-(Piperidin-4-yl)-1H-pyrazolo[3,4-c]pyridin-4-yl)dihydropyrimidine-2,4(1H,3H)-dione (ECB8)

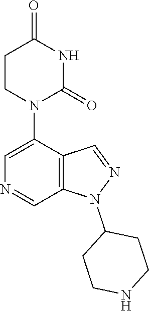 [SMILES: O=C1CCN(c2cncc3c2cnn3C2CCNCC2)C(=O)N1]

tert-Butyl 4-(4-(2,4-dioxo-3-((2-(trimethylsilyl)ethoxy)methyl)tetrahydropyrimidin-1(2H)-yl)-1H-pyrazolo[3,4-c]pyridin-1-yl)piperidine-1-carboxylate (may be prepared as described in Description 23; 5 g, 9.18 mmol) and TFA (15 mL, 195 mmol) in DCM (15 mL) were mixed and stirred for 1 h. The reaction mixture was concentrated using a flow of nitrogen gas, co-evaporated with toluene (twice), treated with 4 N NH₃-MeOH and evaporated to give a cloudy white syrup. The crude product was purified in two batches using a 150 g C18 silica column, eluting with 0-20% MeCN: 10 mM aqueous ammonium carbonate, and the desired fractions were combined and concentrated in vacuo to give the title compound as a white powder (1.8 g, 5.44 mmol, 59% yield). LCMS (high pH A): Rt=0.49 min, MH⁺=315.

### Example CB9

### 1-(6-Chloro-1-(piperidin-4-yl)-1H-indol-4-yl)dihydropyrimidine-2,4(1H,3H)-dione (ECB9)

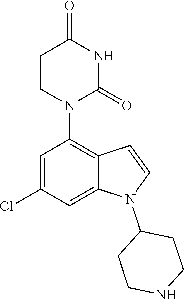 [SMILES: O=C1CCN(c2cc(Cl)cc3c2ccn3C2CCNCC2)C(=O)N1]

A flask containing 10 wt. % palladium on carbon (8 mg, 7.52 μmol) was evacuated and purged with nitrogen (×3) A solution of benzyl 4-(6-chloro-4-(2,4-dioxotetrahydropyrimidin-1(2H)-yl)-1H-indol-1-yl)piperidine-1-carboxylate (may be prepared as described in Description 26; 66 mg, 0.137 mmol) in ethanol (10 mL) was treated with Pd/C (10%) (8 mg, 7.52 μmol) and hydrogenated under atmospheric pressure of hydrogen at room temperature for 16 h. The reaction mixture was filtered through Celite and washed with ethanol (20 mL) and evaporated in vacuo. The material was dissolved in MeOH (5 mL) and purified by means of a MeOH-preconditioned 1 g sulphonic acid (SCX-2) cartridge. The resulting gum was purified by MDAP (high pH) to give after evaporation of the fractions vacuo the title compound as an off white solid (16 mg, 0.046 mmol). LCMS (high pH A): Rt=0.78 min; MH⁺=347.

### Example CB10

### 1-(6-Fluoro-1-(piperidin-4-yl)-1H-indazol-4-yl)dihydropyrimidine-2,4(1H,3H)-dione (ECB10)

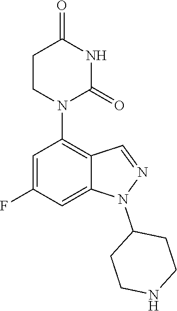 [SMILES: O=C1CCN(c2cc(F)cc3c2cnn3C2CCNCC2)C(=O)N1]

Benzyl 4-(4-amino-6-fluoro-1H-indazol-1-yl)piperidine-1-carboxylate (may be prepared as described in Description 27; 2.1 g, 5.70 mmol) and acrylic acid (1.174 mL, 17.10 mmol) stirred overnight (approximately 16 h) at 60° C. An additional 1 equivalent of acrylic acid was added and the reaction mixture heated at 80° C. for 2 h. The reaction mixture was concentrated under a stream of nitrogen, then taken up in Acetic Acid (10 mL), treated with urea (1.712 g, 28.5 mmol) and heated at 135° C. overnight (approximately 16 h). The reaction mixture was concentrated, partitioned between EtOAc and water. The organic phase was washed with water, sodium bicarbonate solution and brine, dried (MgSO4), filtered and evaporated in vacuo to give a pale brown oil. Purification by flash column chromatography (Silica, 120 g, 30-90% EtOAc/cyclohexane) gave after evaporation of the fractions benzyl 4-(4-(2,4-dioxotetrahydropyrimidin-1(2H)-yl)-6-fluoro-1H-indazol-1-yl)piperidine-1-carboxylate as an impure off-white solid. This was dissolved in ethanol (20 mL), treated with ammonium formate (0.813 g, 12.89 mmol) and Pd—C (10%) (0.137 g, 1.289 mmol) and refluxed under nitrogen for 40 mins. The reaction mixture was cooled, filtered through celite, washing with MeOH and MeCN and evaporated in vacuo to give a pink collapsed foam. Purification by flash column chromatography (C18 silica, 100 g, 0-25% (10 mM pH 10 NH₄CO₂H aq/MeCN)) gave the title compound as a collapsed white foam (220 mg, 0.63 mmol, 49% yield). LCMS (high pH): Rt=0.63 min, MH⁺=332.1.

### Example CB11

### 1-(1-isopropyl-6-(piperazine-1-carbonyl)-1H-indol-4-yl)dihydropyrimidine-2,4(1H,3H)-dione (ECB11)

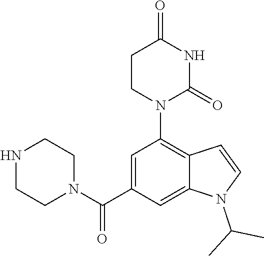 [SMILES: CC(C)n1ccc2c(N3CCC(=O)NC3=O)cc(C(=O)N3CCNCC3)cc21]

A solution of tert-butyl 4-(4-(2,4-dioxotetrahydropyrimidin-1(2H)-yl)-1-isopropyl-1H-indole-6-carbonyl)piperazine-1-carboxylate (may be prepared as described in Description 36; 5.3 g, 10.96 mmol) in DCM (10 mL) was treated with TFA (10 mL) and stirred at ambient temperature for 1 h. The mixture was evaporated to dryness then eluted through a 50 g SCX2 cartridge (primed with MeOH, eluted with 2 column volumes MeOH then with 4 column volumes of 5% triethylamine in MeOH). Product-containing fractions were combined and evaporated to dryness to give the title compound as a pale brown solid (3.80 g, 9.91 mmol, 90% yield). LCMS (high pH A): Rt=0.64 min, MH⁺=384.

### Example CB12

### 1-(1-Isopropyl-6-(2,9-diazaspiro[5.5]undecane-9-carbonyl)-1H-indol-4-yl)dihydropyrimidine-2,4(1H,3H)-dione hydrochloride (ECB12)

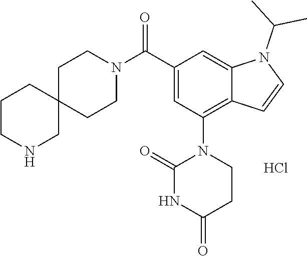 [SMILES: CC(C)n1ccc2c(N3CCC(=O)NC3=O)cc(C(=O)N3CCC4(CCCNC4)CC3)cc21 ; Cl]

1 M HCl in EtOAc (4 mL, 4.00 mmol) was added to a stirred solution of tert-butyl 9-(4-(2,4-dioxotetrahydropyrimidin-1(2H)-yl)-1-isopropyl-1H-indole-6-carbonyl)-2,9-diazaspiro[5.5]undecane-2-carboxylate (may be prepared as described in Description 39; 204 mg, 0.370 mmol) in EtOAc (1.5 mL). The reaction mixture was stirred at room temperature for 4 h. Diethyl ether was added (15 mL) and the mixture was stirred for 15 min. The solvent was decanted off and the residue dried in vacuo to give the title compound as a brown solid (136 mg, 0.279 mmol, 75% yield). LCMS (high pH A): Rt=0.76 min, MH⁺=452.

### Example CB13

### 1-(1-Isopropyl-6-(3,9-diazaspiro[5.5]undecane-3-carbonyl)-1H-indol-4-yl)dihydropyrimidine-2,4(1H,3H)-dione hydrochloride (ECB13)

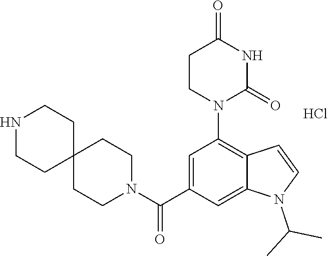 [SMILES: CC(C)n1ccc2c(N3CCC(=O)NC3=O)cc(C(=O)N3CCC4(CCNCC4)CC3)cc21 ; Cl]

1 M HCl in EtOAc (4 mL, 4.00 mmol) was added to a stirred solution of tert-butyl 9-(4-(2,4-dioxotetrahydropyrimidin-1(2H)-yl)-1-isopropyl-1H-indole-6-carbonyl)-3,9-diazaspiro[5.5]undecane-3-carboxylate (may be prepared as described in Description 40; 188 mg, 0.341 mmol) in EtOAc (2 mL). The reaction mixture was stirred at room temperature for 2 h. Diethyl ether (15 mL) was added and the mixture was stirred for 30 min. The solvent was decanted off and the residue dried in vacuo to give the title compound as a brown solid (142 mg, 0.291 mmol, 85% yield). LCMS (high pH A): Rt=0.68 min, MH⁺=452.

### Example CB14

### 1-(1-Isopropyl-6-(2,8-diazaspiro[4.5]decane-2-carbonyl)-1H-indol-4-yl)dihydropyrimidine-2,4(1H,3H)-dione hydrochloride (ECB14)

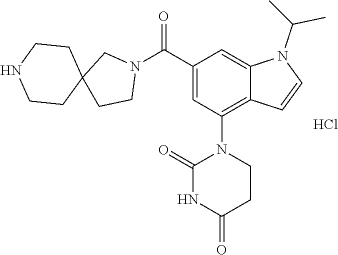 [SMILES: CC(C)n1ccc2c(N3CCC(=O)NC3=O)cc(C(=O)N3CCC4(CCNCC4)C3)cc21 ; Cl]

1 M HCl in EtOAc (4 mL, 4.00 mmol) was added to a stirred solution of tert-butyl 2-(4-(2,4-dioxotetrahydropyrimidin-1(2H)-yl)-1-isopropyl-1H-indole-6-carbonyl)-2,8-diazaspiro[4.5]decane-8-carboxylate (may be prepared as described in Description 41; 49 mg, 0.091 mmol) in EtOAc (1 mL). The reaction mixture was stirred at room temperature for 6 h. Diethyl ether (15 mL) was added and the mixture was stirred for 15 min. The solvent was decanted off and the residue dried in vacuo to give the title compound as a purple solid (34 mg, 0.072 mmol, 79% yield). LCMS (high pH A): Rt=0.67 min, MH⁺=438.

### Example CB15

### 4-(2,4-Dioxotetrahydropyrimidin-1(2H)-yl)-1-isopropyl-N-methyl-N-(piperidin-4-yl)-1H-indole-6-carboxamide (ECB15)

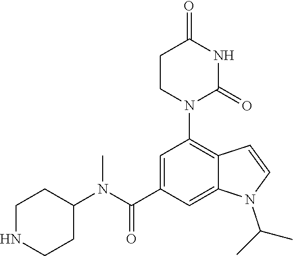 [SMILES: CC(C)n1ccc2c(N3CCC(=O)NC3=O)cc(C(=O)N(C)C3CCNCC3)cc21]

A solution of tert-butyl 4-(4-(2,4-dioxotetrahydropyrimidin-1(2H)-yl)-1-isopropyl-N-methyl-1H-indole-6-carboxamido)piperidine-1-carboxylate (may be prepared as described in Description 37; 2.5 g, 4.89 mmol) in DCM (10 mL) was treated with TFA (5 mL) and stirred at ambient temperature for 2 h. The mixture was evaporated to dryness and the residue was dissolved in MeOH (10 mL) and added to a 20 g aminopropyl solid-phase extraction cartridge which had been pre-conditioned with MeOH. The cartridge was eluted with MeOH (3 column volumes) and the product-containing fractions were combined and evaporated to dryness to give the title compound (1.87 g, 4.54 mmol, 93% yield). LCMS (high pH A): Rt=0.69 min, MH⁺=412.

### Example CB16

### 4-(2,4-Dioxotetrahydropyrimidin-1(2H)-yl)-1-isopropyl-N-methyl-N-(piperidin-4-ylmethyl)-1H-indole-6-carboxamide (ECB16)

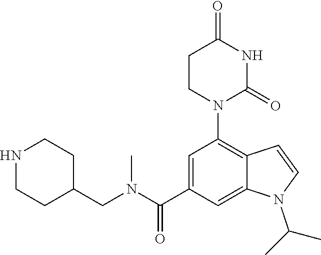 [SMILES: CC(C)n1ccc2c(N3CCC(=O)NC3=O)cc(C(=O)N(C)CC3CCNCC3)cc21]

A solution of tert-butyl 4-((4-(2,4-dioxotetrahydropyrimidin-1(2H)-yl)-1-isopropyl-N-methyl-1H-indole-6-carboxamido)methyl)piperidine-1-carboxylate (may be prepared as described in Description 38; 2.5 g, 4.76 mmol) in DCM (10 mL) was treated with TFA (5 mL) and stirred at ambient temperature for 2 h. The mixture was evaporated to dryness and the residue was dissolved in MeOH (10 mL) and added to a 20 g aminopropyl solid-phase extraction cartridge which had been pre-conditioned with MeOH. The cartridge was eluted with MeOH (3 column volumes) and the product-containing fractions were combined and evaporated to dryness to afford the title compound (1.62 g, 3.81 mmol, 80% yield). LCMS (high pH A): Rt=0.70 min, MH⁺=426.

### Example CB17

### 1-(7-(Piperidin-4-yl)-7H-pyrrolo[2,3-d]pyrimidin-4-yl)dihydropyrimidine-2,4(1H,3H)-dione (ECB17)

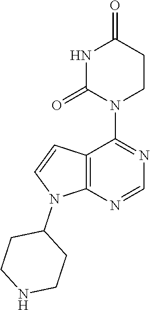 [SMILES: O=C1CCN(c2ncnc3c2ccn3C2CCNCC2)C(=O)N1]

tert-Butyl 4-(4-(2,4-dioxo-3-((2-(trimethylsilyl)ethoxy)methyl)tetrahydropyrimidin-1(2H)-yl)-7H-pyrrolo[2,3-d]pyrimidin-7-yl)piperidine-1-carboxylate (may be prepared as described in Description 173; 1.04 g, 1.909 mmol) in DCM (1 mL) was treated with TFA (2.94 mL, 38.2 mmol) and stirred, causing the starting material to dissolve. After 1 h, the reaction mixture was blown down and held under high vacuum. The material was treated with 4 M NH₃ in MeOH (10 mL) and stirred for 1 h. The mixture was blown down and allowed to stand over the weekend (approximately 60 h). The resulting white solid was dissolved in warm DMSO (4 mL) and purified by flash chromatography on a 100 g C18-silica cartridge eluting with 0-25% MeCN in 10 mM NH₄CO₂H (aq) (pH10). Evaporation of the fractions gave the title compound (520 mg, 1.572 mmol, 82% yield). LCMS (high pH A): Rt=0.52 min, MH⁺=315.

### Example CB18

### 1-(1-(Piperidin-4-yl)-1H-indazol-4-yl)dihydropyrimidine-2,4(1H,3H)-dione (ECB18)

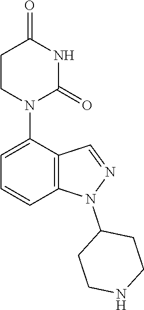 [SMILES: O=C1CCN(c2cccc3c2cnn3C2CCNCC2)C(=O)N1]

A flask containing 10 wt. % palladium on carbon (0.25 g, 0.235 mmol) was evacuated and purged with nitrogen (×3) and a solution of benzyl 4-(4-(2,4-dioxo-3-((2-(trimethylsilyl)ethoxy)methyl)tetrahydropyrimidin-1(2H)-yl)-1H-indazol-1-yl)piperidine-1-carboxylate (may be prepared as described in Description 185; 1.35 g, 2.337 mmol) in ethanol (30 mL) added. The mixture was evacuated and purged with nitrogen (×3) and stirred under atmospheric pressure hydrogen at room temperature for 17 h. The mixture was filtered through Celite and the Celite washed with ethanol (100 mL). The filtrate was evaporated in vacuo to give a light yellow gum (978 mg). The gum was dissolved in DCM (10 mL) and treated with TFA (3 mL, 38.9 mmol) and stirred at RT for 6 h. The mixture was evaporated in vacuo and the residue dissolved in MeOH (5 mL). The solution was treated with 4 M ammonia in MeOH (10 mL, 40.0 mmol) and stirred in a stoppered vessel at room temperature for 3 h. The mixture was evaporated in vacuo and dried under high vacuum for 16 h. The off-white solid (1.4 g) was dissolved in DMSO (5 mL) and was purified by reverse phase flash chromatography on a 60 g C18 cartridge eluting with a 5-50% gradient of MeCN and 10 mM ammonium carbonate in water adjusted to pH10 with ammonia solution over 10 column volumes. The appropriate fractions were combined and the solvent evaporated in vacuo. The solid was dried in a vacuum oven to give the title compound as a white solid (407 mg, 1.299 mmol). LCMS (high pH): Rt=0.57 min, MH⁺=314.

### Example CB19

### 1-(1-(Piperidin-4-yl)-1H-pyrazolo[3,4-b]pyridin-4-yl)dihydropyrimidine-2,4(1H,3H)-dione (ECB19)

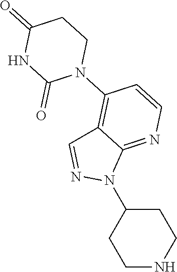 [SMILES: O=C1CCN(c2ccnc3c2cnn3C2CCNCC2)C(=O)N1]

Benzyl 4-(4-(2,4-dioxotetrahydropyrimidin-1(2H)-yl)-1H-pyrazolo[3,4-b]pyridin-1-yl)piperidine-1-carboxylate (may be prepared as described in Description 189; 1 g, 2.230 mmol), ethanol (20 mL) ammonium formate (1.406 g, 22.30 mmol) and Pd—C, 10% (0.237 g, 2.230 mmol) were refluxed under nitrogen for 40 mins. The reaction mixture was cooled and filtered through Celite, washing with DCM and ethanol. The effluent was evaporated in vacuo to give a very insoluble white powder. Very hot DMSO was used to get most of the material into solution, which was purified using MDAP (high pH) to give 1-(1-(piperidin-4-yl)-1H-pyrazolo[3,4-b]pyridin-4-yl)dihydropyrimidine-2,4(1H,3H)-dione (345 mg, 1.010 mmol, 45.3% yield). LCMS (high pH A): Rt=0.51 min, MH⁺=315.1

### Example CB20

### 1-(1-(Azetidin-3-yl)-1H-indol-4-yl)dihydropyrimidine-2,4(1H,3H)-dione (ECB20)

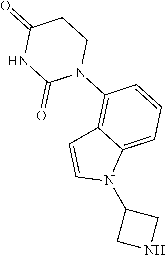 [SMILES: O=C1CCN(c2cccc3c2ccn3C2CNC2)C(=O)N1]

To tert-butyl 3-(4-(2,4-dioxo-3-((2-(trimethylsilyl)ethoxy)methyl)tetrahydropyrimidin-1(2H)-yl)-1H-indol-1-yl)azetidine-1-carboxylate (may be prepared as described in Description 197; 430 mg, 0.835 mmol) was added TFA (1.3 mL, 16.87 mmol), and the reaction mixture was stirred at room temperature for 1.5 h. The solvent was removed in vacuo and the residue cooled in an ice-water bath. Ammonia (4 M in MeOH) (5.9 mL, 23.60 mmol) was added, and the solution was stirred in an open vessel an ice-water bath for 5 min. The mixture was allowed to warm to room temperature and stirred for 1 h. The mixture was concentrated in vacuo to give crude material which was dissolved in 1:1 DMSO:methanol (3 mL) and purified by MDAP (high pH) to give 1-(1-(azetidin-3-yl)-1H-indol-4-yl)dihydropyrimidine-2,4(1H,3H)-dione (78.4 mg, 0.276 mmol, 33.0% yield). LCMS (high pH A): Rt=0.51 min, MH⁺=285.1

### Example CB21

### 1-(5-Fluoro-1-(piperidin-4-yl)-1H-indazol-4-yl)dihydropyrimidine-2,4(1H,3H)-dione (ECB21)

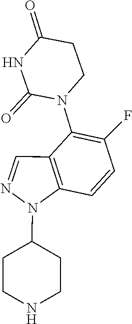 [SMILES: O=C1CCN(c2c(F)ccc3c2cnn3C2CCNCC2)C(=O)N1]

A solution of tert-butyl 4-(4-(2,4-dioxo-3-((2-(trimethylsilyl)ethoxy)methyl)tetrahydropyrimidin-1(2H)-yl)-5-fluoro-1H-indazol-1-yl)piperidine-1-carboxylate (may be prepared as described in Description 215; 957 mg, 1.704 mmol) in DCM (8 mL) was treated with TFA (2.0 mL, 26.0 mmol) and left to stand in a stoppered vessel for 3 h. The mixture was evaporated in vacuo and the residue dissolved in methanol (8.00 mL) and treated with 4 M ammonia in methanol (12 mL, 48.0 mmol). The solution was stirred in a stoppered vessel at room temperature for 1 h and evaporated in vacuo. The gum was loaded and preabsorbed on Florisil (from methanol), and purified on a 24 g silica cartridge using a gradient of 0-20% methanol in DCM (containing 1% NEt₃) to give partially pure product. This material was purified by MDAP (high pH) to give 1-(5-fluoro-1-(piperidin-4-yl)-1H-indazol-4-yl)dihydropyrimidine-2,4(1H,3H)-dione (62 mg, 0.187 mmol, 11% yield). LCMS (high pH A): Rt=0.63 min, MH⁺=332

### Example CB22

### 1-(1-(Piperidin-4-yl)-1H-indol-4-yl)pyrimidine-2,4(1H,3H)-dione (ECB22)

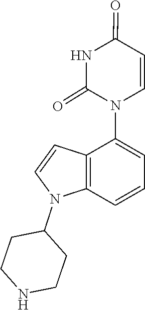 [SMILES: O=c1ccn(-c2cccc3c2ccn3C2CCNCC2)c(=O)[nH]1]

To tert-butyl 4-(4-(2,4-dioxo-3-((2-(trimethylsilyl)ethoxy)methyl)-3,4-dihydropyrimidin-1(2H)-yl)-1H-indol-1-yl)piperidine-1-carboxylate (may be prepared as described in Description 219; 1.2 g, 2.219 mmol) in DCM (15 mL) at 0° C. was added into TFA (6 mL, 78 mmol). After 1 h, the reaction was concentrated, quenched with 7M ammonia in methanol (5 mL) and concentrated. The residue was purified via reverse-phase chromatography, eluting with 9:1 10 mM ammonium bicarbonate in water:MeCN. Product was isolated and treated with water (50 mL) and extracted with 20% methanol in DCM (2×100 mL). The combined organics were dried over sodium sulphate and concentrated to give 1-(1-(piperidin-4-yl)-1H-indol-4-yl)pyrimidine-2,4(1H,3H)-dione (201 mg, 0.634 mmol, 28.6% yield). LCMS (high formic A): Rt=0.318 min, MH⁺=311.0

### Example CB23

### 1-(1-((3R,4R)-3-Fluoropiperidin-4-yl)-1H-indol-4-yl)dihydropyrimidine-2,4(1H,3H)-dione (ECB23)

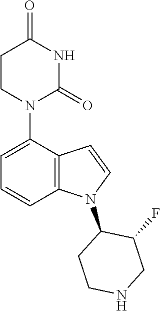 [SMILES: O=C1CCN(c2cccc3c2ccn3[C@@H]2CCNC[C@H]2F)C(=O)N1]

To tert-butyl (3R,4R)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (may be prepared as described in Description 222; 350 mg, 0.531 mmol) in DCM (20 mL) was added TFA (817 μl, 10.61 mmol), and the reaction mixture was stirred at room temperature for 1.5 h. The solvent was removed in vacuo, and the residue cooled in an ice-water bath. Ammonia (4 M in methanol) (3714 μl, 14.86 mmol) was added, and the solution was stirred in an open vessel in the ice-water bath for 5 min. The mixture was allowed to warm to room temperature and stirred for 1.5 h. The mixture was concentrated in vacuo, and the crude was dissolved in 1:1 DMSO:methanol (3 mL) and purified by MDAP (high pH) to give as a light brown solid. This material was further purified on a 12 g Redisep silica column, eluting with 0-75% (20% methanol in DCM) in DCM to give 1-(1-((3R,4R)-3-fluoropiperidin-4-yl)-1H-indol-4-yl)dihydropyrimidine-2,4(1H,3H)-dione (87.5 mg, 0.225 mmol, 42.4% yield). LCMS (high pH A): Rt=0.67 min, MH⁺=331.1

### Example CB24

### 1-(1-((3S,4S)-3-Fluoropiperidin-4-yl)-1H-indol-4-yl)dihydropyrimidine-2,4(1H,3H)-dione (ECB24)

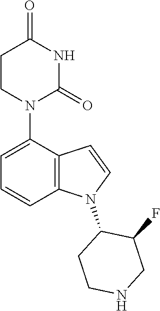 [SMILES: O=C1CCN(c2cccc3c2ccn3[C@H]2CCNC[C@@H]2F)C(=O)N1]

To tert-butyl (3S,4S)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (may be prepared as described in Description 225; 500 mg, 0.892 mmol) was added TFA (1400 μl, 18.17 mmol), and the reaction mixture was stirred at room temperature for 1 h. The solvent was removed in vacuo, and the residue cooled in an ice-water bath. Ammonia (4 M in methanol) (6.24 ml, 24.97 mmol) was added, and the solution was stirred in an open vessel in the ice-water bath for 5 min. The mixture was allowed to warm to room temperature and stirred for 40 min. The mixture was concentrated in vacuo, and the crude was dissolved in 1:1 DMSO:methanol (5 mL) and purified by MDAP (high pH) to give as a light brown solid. This material was further purified on a 12 g Redisep silica column, eluting with 0-75% (20% methanol in DCM) in DCM to give 1-(1-((3S,4S)-3-fluoropiperidin-4-yl)-1H-indol-4-yl)dihydropyrimidine-2,4(1H,3H)-dione (213 mg, 0.645 mmol, 81.5% yield). LCMS (high pH A): Rt=0.67 min, MH⁺=331.1

### Example CB25

### 1-(1-(Piperidin-4-yl)-1H-pyrrolo[3,2-c]pyridin-4-yl)dihydropyrimidine-2,4(1H,3H)-dione (ECB25)

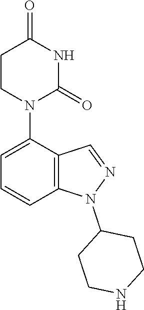 [SMILES: O=C1CCN(c2cccc3c2cnn3C2CCNCC2)C(=O)N1]

To tert-butyl 4-(4-(2,4-dioxo-3-((2-(trimethylsilyl)ethoxy)methyl)tetrahydropyrimidin-1(2H)-yl)-1H-pyrrolo[3,2-c]pyridin-1-yl)piperidine-1-carboxylate (may be prepared as described in Description 231; 1.8 g, 3.31 mmol) in DCM (1 mL) was added TFA (5.10 mL, 66.2 mmol). After 1 h the reaction was blown down and held under high vacuum, then treated with 4M NH₃ in methanol (10 ml). After 1 h, the mixture was blown down, and the resulting white solid was dissolved in warm DMSO (4 ml) and purified by flash chromatography (C18-silica, 100 g), eluting with 0-25% 10 mM pH10 NH₄CO₂H/aq in MeCN to give 1-(1-(piperidin-4-yl)-1H-pyrrolo[3,2-c]pyridin-4-yl)dihydropyrimidine 2,4(1H,3H)-dione (0.9 g, 2.73 mmol, 82% yield). LCMS (high pH A): Rt=0.54 min, MH⁺=314.1

### Example CB26

### 1-(1-(Piperidin-4-yl)-1H-pyrrolo[2,3-b]pyridin-4-yl)dihydropyrimidine-2,4(1H,3H)-dione (ECB26)

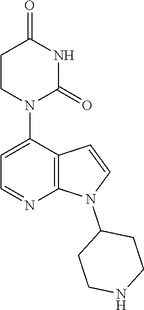 [SMILES: O=C1CCN(c2ccnc3c2ccn3C2CCNCC2)C(=O)N1]

To tert-butyl 4-(4-(2,4-dioxo-3-((2-(trimethylsilyl)ethoxy)methyl)tetrahydropyrimidin-1(2H)-yl)-1H-pyrrolo[2,3-b]pyridin-1-yl)piperidine-1-carboxylate (may be prepared as described in Description 233; 1.4 g, 2.57 mmol) in DCM (1 mL) was added TFA (3.97 mL, 51.5 mmol). After 1 h the reaction was blown down and held under high vacuum, then treated with 4M NH₃ in methanol (10 ml). After 1 h, the mixture was blown down, then treated with 4M NH₃ in methanol (10 ml) and stirred 1 h. The resulting white solid was collected by filtration and washed with a small amount of methanol and ether, then dried in vacuo to give 1-(1-(piperidin-4-yl)-1H-pyrrolo[2,3-b]pyridin-4-yl)dihydropyrimidine-2,4(1H,3H)-dione (0.7 g, 2.122 mmol, 82% yield). LCMS (high pH A): Rt=0.54 min, MH⁺=314.1

### Example CB27

### 1-(1-((3S,4R)-3-Fluoropiperidin-4-yl)-1H-indol-4-yl)dihydropyrimidine-2,4(1H,3H)-dione (ECB27)

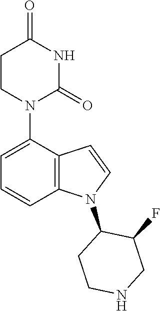 [SMILES: O=C1CCN(c2cccc3c2ccn3[C@@H]2CCNC[C@@H]2F)C(=O)N1]

To tert-butyl (3S,4R)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (may be prepared as described in Description 236; 414 mg, 0.738 mmol) was added TFA (1140 μl, 14.80 mmol), and the reaction mixture was stirred at room temperature for 1.5 h. The solvent was removed in vacuo, and the residue cooled in an ice-water bath. Ammonia (4 M in methanol) (5.17 ml, 20.67 mmol) was added, and the solution was stirred in an open vessel in the ice-water bath for 5 min. The mixture was allowed to warm to room temperature and stirred for 2.5 h. The mixture was concentrated in vacuo, and the crude was dissolved in 1:1 DMSO:methanol (3 mL) and purified by MDAP (high pH) to give as a light brown solid. This material was further purified on a 12 g Redisep silica column, eluting with 0-75% (20% methanol in DCM) in DCM to give 1-(1-((3S,4R)-3-fluoropiperidin-4-yl)-1H-indol-4-yl)dihydropyrimidine-2,4(1H,3H)-dione (151 mg, 0.457 mmol, 61.9% yield). LCMS (high pH A): Rt=0.63 min, MH⁺=331.1

### Example CB28

### 1-(1-((3R,4S)-3-Fluoropiperidin-4-yl)-1H-indol-4-yl)dihydropyrimidine-2,4(1H,3H)-dione (ECB28)

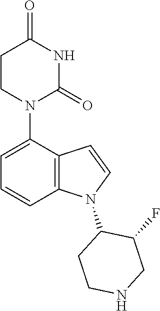

To tert-butyl (3R,4S)-4-(4-(2,4-dioxo-3-((2-(trimethylsilyl)ethoxy)methyl)tetrahydropyrimidin-1(2H)-yl)-1H-indol-1-yl)-3-fluoropiperidine-1-carboxylate (may be prepared as described in Description 239; 407 mg, 0.726 mmol) was added TFA (1.12 ml, 14.54 mmol), and the reaction mixture was stirred at room temperature for 1.5 h. The solvent was removed in vacuo, and the residue cooled in an ice-water bath. Ammonia (4 M in methanol) (5.08 mL, 20.32 mmol) was added, and the solution was stirred in an open vessel in the ice-water bath for 5 min. The mixture was allowed to warm to room temperature and stirred for 3 h. The mixture was concentrated in vacuo, and the crude was dissolved in 1:1 DMSO:methanol (3 mL) and purified by MDAP (high pH) to give 1-(1-((3R,4S)-3-fluoropiperidin-4-yl)-1H-indol-4-yl)dihydropyrimidine-2,4(1H,3H)-dione (141 mg, 0.427 mmol, 58.8% yield). LCMS (high pH A): Rt=0.62 min, MH⁺=331.1

### Example CB29

### (S)-1-(1-((3-Fluoropiperidin-3-yl)methyl)-1H-indol-4-yl)dihydropyrimidine-2,4(1H,3H)-dione (ECB29)

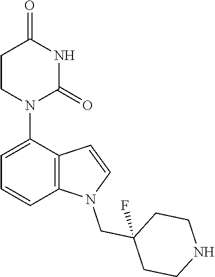

To tert-butyl (R)-3-((4-(2,4-dioxo-3-((2-(trimethylsilyl)ethoxy)methyl)tetrahydropyrimidin-1(2H)-yl)-1H-indol-1-yl)methyl)-3-fluoropiperidine-1-carboxylate (may be prepared as described in Description 246; 0.60 g, 1.044 mmol) in DCM was added TFA (0.080 mL, 1.044 mmol). After 2 h, the solvent was evaporated in vacuo, and the residue was dissolved in methanolic ammonia (2M, 20 ml). After 2 h, the solvent was evaporated in vacuo. This material was dissolved in water (10 ml), and the mixture was saturated with sodium bicarbonate, then extracted with 10% methanol in DCM (3×10 ml). The combined organics were dried and evaporated in vacuo and purified over a 40 g silica column, eluting with 0-20% 2M methanolic ammonia in cyclohexane to give(S)-1-(1-((3-fluoropiperidin-3-yl)methyl)-1H-indol-4-yl)dihydropyrimidine-2,4(1H,3H)-dione (120 mg, 0.348 mmol, 33.4% yield). LCMS (high pH A): Rt=0.71 min, MH⁺=345.1

### Example CB30

### 1-(1-(Piperidin-4-yl)-6-(trifluoromethyl)-1H-indazol-4-yl)dihydropyrimidine-2,4(1H,3H)-dione (ECB30)

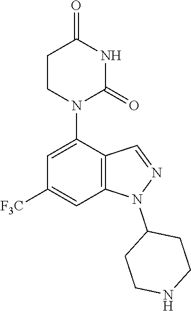 [SMILES: O=C1CCN(c2cc(C(F)(F)F)cc3c2cnn3C2CCNCC2)C(=O)N1]

To tert-butyl 4-(4-(2,4-dioxo-3-((2-(trimethylsilyl)ethoxy)methyl)tetrahydropyrimidin-1(2H)-yl)-6-(trifluoromethyl)-1H-indazol-1-yl)piperidine-1-carboxylate (may be prepared as described in Description 251; 600 mg, 0.981 mmol) in DCM (1 mL) was added TFA (2 mL, 26.0 mmol), and the reaction mixture was stirred at room temperature for 1 h. The solvent was removed in vacuo, and the residue treated with ammonia (4 M in methanol) (2 mL). The solution was stirred for 1 h, concentrated and purified by reverse-phase chromatography eluting, with 10-50% 10 mM pH10 NH₄CO₂ (aq) in MeCN to give 1-(1-(piperidin-4-yl)-6-(trifluoromethyl)-1H-indazol-4-yl)dihydropyrimidine-2,4(1H,3H)-dione (300 mg, 0.747 mmol, 76% yield). LCMS (high pH A): Rt=0.79 min, MH⁺=382.2

### Example CB31

### 1-(1-((4-Fluoropiperidin-4-yl)methyl)-1H-indol-4-yl)dihydropyrimidine-2,4(1H,3H)-dione (ECB31)

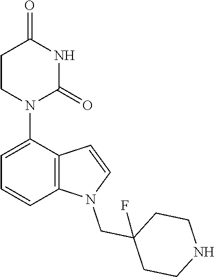 [SMILES: O=C1CCN(c2cccc3c2ccn3CC2(F)CCNCC2)C(=O)N1]

4M Hydrogen chloride in dioxane (2 mL) was added to tert-butyl 4-((4-(2,4-dioxo-3-((2-(trimethylsilyl)ethoxy)methyl)tetrahydropyrimidin-1(2H)-yl)-1H-indol-1-yl)methyl)-4-fluoropiperidine-1-carboxylate (may be prepared as described in Description 263; 135 mg, 0.235 mmol). After 2 hours the solvent was evaporated, and the residue was azeotroped with toluene. 7M Ammonia in methanol (1 mL) was added, and the mixture was stirred at room temperature for 2 hours. The solvent was evaporated, and the residue was suspended in ethanol (2 mL) and stirred for 5 minutes. The mixture was filtered, and the solvent was evaporated to give 1-(1-((4-fluoropiperidin-4-yl)methyl)-1H-indol-4-yl)dihydropyrimidine-2,4(1H,3H)-dione (68 mg, 0.197 mmol, 84% yield). LCMS (formic A): Rt=0.38 min, MH⁺=345.0

### Example CB32

### 1-(5-Fluoro-1-(piperidin-4-yl)-1H-indol-4-yl)dihydropyrimidine-2,4(1H,3H)-dione (ECB32)

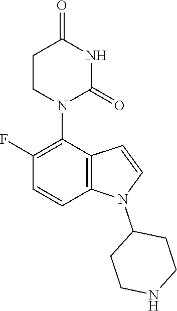 [SMILES: O=C1CCN(c2c(F)ccc3c2ccn3C2CCNCC2)C(=O)N1]

tert-Butyl 4-(4-(2,4-dioxo-3-((2-(trimethylsilyl)ethoxy)methyl)tetrahydropyrimidin-1(2H)-yl)-5-fluoro-1H-indol-1-yl)piperidine-1-carboxylate (may be prepared as described in Description 268; 96 mg, 0.171 mmol) in DCM (0.50 mL) was treated with TFA (0.20 mL, 2.60 mmol). After 3 h, the reaction was evaporated under a stream of nitrogen, and the residue was dissolved in methanol (0.50 mL). 4 M Ammonia in methanol (0.111 mL, 5.14 mmol) was added, and after 1 h, the reaction was evaporated under a stream of nitrogen. The residue purified by reverse phase flash chromatography on a 30 g C18 cartridge eluting with a 0-55% gradient of acetonitrile (containing 1% 0.88 NH_{3(aq)}) and 10 mM ammonium carbonate in water adjusted to pH 10 with ammonia to give 1-(5-Fluoro-1-(piperidin-4-yl)-1H-indol-4-yl)dihydropyrimidine-2,4(1H,3H)-dione (30 mg, 0.091 mmol). LCMS (high pH A): Rt=0.70 min, MH⁺=331

### Example CB33

### (R)-1-(1-(Pyrrolidin-3-yl)-1H-indol-4-yl)dihydropyrimidine-2,4(1H,3H)-dione (ECB33)

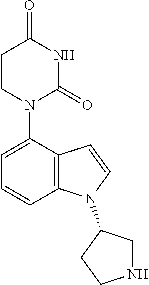 [SMILES: O=C1CCN(c2cccc3c2ccn3[C@H]2CCNC2)C(=O)N1]

To tert-butyl (R)-3-(4-(2,4-dioxo-3-((2-(trimethylsilyl)ethoxy)methyl)tetrahydropyrimidin-1(2H)-yl)-1H-indol-1-yl)pyrrolidine-1-carboxylate (may be prepared as described in Description 270; 211 mg, 0.399 mmol) was added TFA (0.631 mL, 8.18 mmol). After 40 min, the reaction was concentrated in vacuo, cooled in an ice-water bath and ammonia (4 M in methanol) (2.8 mL, 11.20 mmol) was added. After 5 min, the mixture was allowed to warm to room temperature. After 1 h, the mixture was concentrated in vacuo, dissolved in 1:1 DMSO:methanol (2 mL), loaded onto a preconditioned Redisep gold 50 g HP C18 column, eluting with 5-35% MeCN in 10 mM ammonium carbonate (pH10) to give (R)-1-(1-(pyrrolidin-3-yl)-1H-indol-4-yl)dihydropyrimidine-2,4(1H,3H)-dione (113 mg, 0.379 mmol, 95%). LCMS (high pH A): Rt=0.60 min, MH⁺=299.1

### Example CB34

### (S)-1-(1-(Pyrrolidin-3-yl)-1H-indol-4-yl)dihydropyrimidine-2,4(1H,3H)-dione (ECB34)

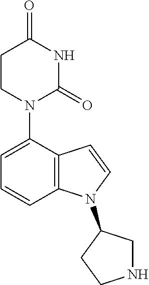 [SMILES: O=C1CCN(c2cccc3c2ccn3[C@@H]2CCNC2)C(=O)N1]

To tert-butyl(S)-3-(4-(2,4-dioxo-3-((2-(trimethylsilyl)ethoxy)methyl)tetrahydropyrimidin-1(2H)-yl)-1H-indol-1-yl)pyrrolidine-1-carboxylate (may be prepared as described in Description 272; 243 mg, 0.460 mmol) was added TFA (0.726 mL, 9.43 mmol). After 50 min, the reaction was concentrated in vacuo, cooled in an ice-water bath and ammonia (4 M in methanol) (3.2 mL, 12.80 mmol) was added. After 5 min, the mixture was allowed to warm to room temperature. After 1 h, the mixture was concentrated in vacuo, dissolved in 1:1 DMSO:methanol (2 mL), loaded onto a preconditioned Redisep gold 50 g HP C18 column, eluting with 5-35% MeCN in 10 mM ammonium carbonate (pH10) to give (S)-1-(1-(pyrrolidin-3-yl)-1H-indol-4-yl)dihydropyrimidine-2,4(1H,3H)-dione (94 mg, 0.315 mmol, 68.6%). LCMS (high pH A): Rt=0.60 min, MH⁺=299.0

### Example CB35

### 1-(1-(2-Azaspiro[3.3]heptan-6-yl)-1H-indol-4-yl)dihydropyrimidine-2,4(1H,3H)-dione (ECB35)

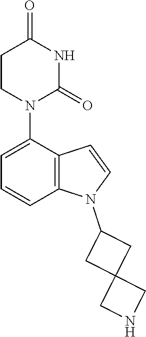 [SMILES: O=C1CCN(c2cccc3c2ccn3C2CC3(CNC3)C2)C(=O)N1]

To tert-butyl 6-(4-(2,4-dioxo-3-((2-(trimethylsilyl)ethoxy)methyl)tetrahydropyrimidin-1(2H)-yl)-1H-indol-1-yl)-2-azaspiro[3.3]heptane-2-carboxylate (may be prepared as described in Description 274; 422 mg, 0.761 mmol) was added TFA (1.2 mL, 15.58 mmol). After 1.5 h, the reaction was concentrated in vacuo, cooled in an ice-water bath and ammonia (4 M in methanol) (5.32 mL, 21.30 mmol) was added. after 5 min, the mixture was allowed to warm to room temperature. After 1 h, the mixture was concentrated in vacuo, dissolved in 1:1 DMSO:methanol (4 mL), loaded onto a preconditioned Redisep gold 100 g HP C18 Aq column, eluting with 5-40% MeCN in 10 mM ammonium carbonate (pH10) to give 1-(1-(2-azaspiro[3.3]heptan-6-yl)-1H-indol-4-yl)dihydropyrimidine-2,4(1H,3H)-dione (83 mg, 0.128 mmol, 16.82% yield). LCMS (high pH A): Rt=0.62 min, MH⁺=325.1

### Example CB36

### 1-(2-Chloro-7-(piperidin-4-yl)-7H-pyrrolo[2,3-d]pyrimidin-4-yl)dihydropyrimidine-2,4(1H,3H)-dione (ECB36)

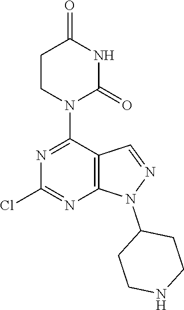 [SMILES: O=C1CCN(c2nc(Cl)nc3c2cnn3C2CCNCC2)C(=O)N1]

1-(2-Chloro-7-(piperidin-4-yl)-7H-pyrrolo[2,3-d]pyrimidin-4-yl)-3-((2-(trimethylsilyl)ethoxy)methyl)dihydropyrimidine-2,4(1H,3H)-dione (may be prepared as described in Description 279; 400 mg, 0.835 mmol) in DCM (1 mL) was treated with TFA (1.287 mL, 16.70 mmol). After 1 h, the reaction was concentrated and treated with 4M NH₃ in methanol (10 ml). After 1 h, the reaction was concentrated, dissolved in warm DMSO (2 ml) and purified over C18-silica, 50 g, eluting with 0-25% 10 mM pH10 NH₄CO₂H (aq) in MeCN to give 1-(2-chloro-7-(piperidin-4-yl)-7H-pyrrolo[2,3-d]pyrimidin-4-yl)dihydropyrimidine-2,4(1H,3H)-dione (200 mg, 0.545 mmol, 65.2% yield). LCMS (high pH A): Rt=0.69 min, MH⁺=349.1, 351.1

### Example CB37

### 1-(1-(Piperidin-4-yl)-6-(trifluoromethyl)-1H-indol-4-yl)dihydropyrimidine-2,4(1H,3H)-dione (ECB37)

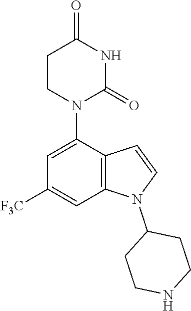 [SMILES: O=C1CCN(c2cc(C(F)(F)F)cc3c2ccn3C2CCNCC2)C(=O)N1]

Benzyl 4-(4-(2,4-dioxotetrahydropyrimidin-1(2H)-yl)-6-(trifluoromethyl)-1H-indol-1-yl)piperidine-1-carboxylate (may be prepared as Described in Description 283; 1.6 g, 3.11 mmol), Pd/C (0.331 g, 3.11 mmol) and ammonium formate (1.961 g, 31.1 mmol) in ethanol (20 mL) were heated at 90° C. for 1 h, filtered, concentrated and purified over C18 silica, 100 g, eluting with 10-50% MeCN in 10 mM pH10 NH₄CO₂H (aq) to give 1-(1-(piperidin-4-yl)-6-(trifluoromethyl)-1H-indol-4-yl)dihydropyrimidine-2,4(1H,3H)-dione (0.7 g, 1.748 mmol, 56.2% yield). LCMS (high pH A): Rt=0.83 min, MH⁺=381.1

### Example CB38

### 1-(3-(Piperidin-4-yl) imidazo[1,5-a]pyridin-8-yl)dihydropyrimidine-2,4(1H,3H)-dione (ECB38)

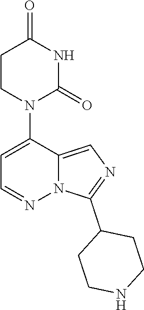 [SMILES: O=C1CCN(c2ccnn3c(C4CCNCC4)ncc23)C(=O)N1]

4M Hydrogen chloride in dioxane (4 mL, 16 mmol) was added to tert-butyl 4-(8-(2,4-dioxo-3-((2-(trimethylsilyl)ethoxy)methyl)tetrahydropyrimidin-1(2H)-yl) imidazo[1,5-a]pyridin-3-yl)piperidine-1-carboxylate (may be prepared as described in Description 287; 310 mg, 0.57 mmol) in dioxane (1 mL). After 2 hours, the reaction was suspended in methanol (1 mL), and 7M ammonia in methanol (4 mL) was added dropwise (fumes). After 3 hours, the solvent was evaporated, and the residue was triturated with diethyl ether (×2) to give 1-(3-(piperidin-4-yl) imidazo[1,5-a]pyridin-8-yl)dihydropyrimidine-2,4(1H,3H)-dione (179 mg, 0.570 mmol, 100% yield). LCMS (high pH A): Rt=0.50 min, MH⁺=313.9

### Example CB39

### 1-(1-(2-(Piperidin-4-yl)ethyl)-1H-indol-4-yl)dihydropyrimidine-2,4(1H,3H)-dione (ECB39)

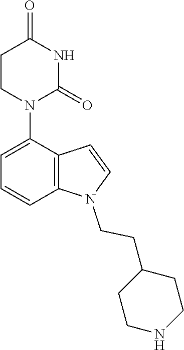 [SMILES: O=C1CCN(c2cccc3c2ccn3CCC2CCNCC2)C(=O)N1]

TFA (4.459 mL, 57.9 mmol) was added to tert-butyl 4-(2-(4-(2,4-dioxo-3-((2-(trimethylsilyl)ethoxy)methyl)tetrahydropyrimidin-1(2H)-yl)-1H-indol-1-yl)ethyl)piperidine-1-carboxylate (may be prepared as described in Description 292; 1620.9 mg, 2.84 mmol). After 1 h, the solvent was removed in vacuo and the residue was cooled in an ice-water bath. Ammonia (4M in methanol, 1.721 mL, 80 mmol) was added, and the reaction was stirred for 5 min. The mixture was allowed to warm to room temperature. After 40 min, the reaction was concentrated in vacuo, dissolved in 1:1 DMSO:methanol (4 mL) and loaded onto a Redisep C18aq gold column, eluting with 0-15% MeCN in 10 mM ammonium bicarbonate, then increased to 40% MeCN and 95% MeCN to yield impure product. This material was dissolved in 1:1 methanol:DMSO 0.5 mL and purified on Waters XSelect CSH C18 30×150 mm 5 μm column, eluting with MeCN in water with an ammonium carbonate modifier to give 1-(1-(2-(piperidin-4-yl)ethyl)-1H-indol-4-yl)dihydropyrimidine-2,4(1H,3H)-dione (37.8 mg, 0.111 mmol, 3.91% yield). LCMS (high pH A): Rt=0.71 min, MH⁺=341.15

### Example CB40

### (R)-1-(1-(Piperidin-3-yl)-1H-indol-4-yl)dihydropyrimidine-2,4(1H,3H)-dione (ECB40)

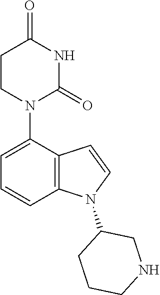

TFA (480 μl, 6.23 mmol) was added to tert-butyl (R)-3-(4-(2,4-dioxo-3-((2-(trimethylsilyl)ethoxy)methyl)tetrahydropyrimidin-1(2H)-yl)-1H-indol-1-yl)piperidine-1-carboxylate (may be prepared as described in Description 294; 164 mg, 0.302 mmol). After 1 h, the solvent was removed in vacuo and the residue was cooled in an ice-water bath. Ammonia (4M in methanol, 2.1 mL, 8.40 mmol) was added, and the reaction was stirred for 5 min. The mixture was allowed to warm to room temperature. After 2 h, the reaction was concentrated in vacuo, dissolved in 1:1 DMSO:methanol (1.25 mL) and loaded onto a preconditioned Redisep gold 30 g HP C18 column, eluting with 5-10% MeCN in 10 mM ammonium bicarbonate to give (R)-1-(1-(piperidin-3-yl)-1H-indol-4-yl)dihydropyrimidine-2,4(1H,3H)-dione (73 mg, 0.234 mmol, 77% yield). LCMS (high pH A): Rt=0.69 min, MH⁺=313.1

### Example CB41

### (S)-1-(1-(Piperidin-3-yl)-1H-indol-4-yl)dihydropyrimidine-2,4(1H,3H)-dione (ECB41)

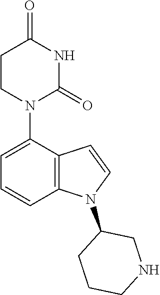 [SMILES: O=C1CCN(c2cccc3c2ccn3[C@@H]2CCCNC2)C(=O)N1]

TFA (520 μl, 6.75 mmol) was added to tert-butyl(S)-3-(4-(2,4-dioxo-3-((2-(trimethylsilyl)ethoxy)methyl)tetrahydropyrimidin-1(2H)-yl)-1H-indol-1-yl)piperidine-1-carboxylate (may be prepared as described in Description 296; 179 mg, 0.330 mmol). After 1 h, the solvent was removed in vacuo and the residue was cooled in an ice-water bath. Ammonia (4M in methanol, 2.3 mL, 9.20 mmol) was added, and the reaction was stirred for 5 min. The mixture was allowed to warm to room temperature. After 2 h, the reaction was concentrated in vacuo, dissolved in 1:1 DMSO:methanol (2 mL) and loaded onto a preconditioned Redisep gold 30 g HP C18 column, eluting with 5-40% MeCN in 10 mM ammonium bicarbonate to give(S)-1-(1-(piperidin-3-yl)-1H-indol-4-yl)dihydropyrimidine-2,4(1H,3H)-dione (87 mg, 0.279 mmol, 84% yield). LCMS (high pH A): Rt=0.69 min, MH⁺=313.1

### Example CB42

### 1-(1-(7-Azaspiro[3.5]nonan-2-yl)-1H-indol-4-yl)dihydropyrimidine-2,4(1H,3H)-dione (ECB42)

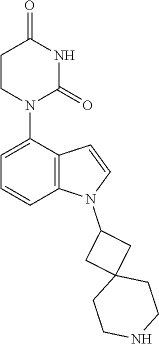 [SMILES: O=C1CCN(c2cccc3c2ccn3C2CC3(CCNCC3)C2)C(=O)N1]

TFA (0.90 ml, 11.68 mmol) was added to tert-butyl 2-(4-(2,4-dioxo-3-((2-(trimethylsilyl)ethoxy)methyl)tetrahydropyrimidin-1(2H)-yl)-1H-indol-1-yl)-7-azaspiro[3.5]nonane-7-carboxylate (may be prepared as described in Description 298; 333 mg, 0.571 mmol). After 1 h, the solvent was removed in vacuo and the residue was cooled in an ice-water bath. Ammonia (4M in methanol, 4.0 ml, 16.00 mmol) was added, and the reaction was stirred for 5 min. The mixture was allowed to warm to room temperature. After 2 h, the reaction was concentrated in vacuo, dissolved in 1:1 DMSO:methanol (2.5 mL) and loaded onto a preconditioned Redisep gold 100 g HP C18 column, eluting with 15-50% MeCN in 10 mM ammonium bicarbonate (pH 10) to give 1-(1-(7-azaspiro[3.5]nonan-2-yl)-1H-indol-4-yl)dihydropyrimidine-2,4(1H,3H)-dione (123 mg, 0.297 mmol, 51.9% yield). LCMS (high pH A): Rt=0.76 min, MH⁺=353.2

### Example CB43

### 1-(1-(1-(Piperidin-4-yl)ethyl)-1H-indol-4-yl)dihydropyrimidine-2,4(1H,3H)-dione (ECB43)

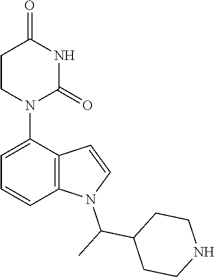 [SMILES: CC(C1CCNCC1)n1ccc2c(N3CCC(=O)NC3=O)cccc21]

To tert-butyl 4-(1-(4-(2,4-dioxo-3-((2-(trimethylsilyl)ethoxy)methyl)tetrahydropyrimidin-1(2H)-yl)-1H-indol-1-yl)ethyl)piperidine-1-carboxylate (may be prepared as Described in Description 308; 160 mg, 0.280 mmol) was added trifluoroacetic acid (443 μl, 5.75 mmol). After 50 min, the reaction was concentrated, cooled in an ice-water bath and treated with ammonia (4 M in methanol) (1962 μl, 7.85 mmol). After 5 min, the mixture was allowed to warm to room temperature, stirred for 1 h, concentrated, dissolved in 1:1 DMSO:methanol (2 mL) and loaded onto a preconditioned Redisep gold 30 g HP C18 column, eluting with 5-40% MeCN in 10 mM ammonium carbonate (pH10) to give 1-(1-(1-(piperidin-4-yl)ethyl)-1H-indol-4-yl)dihydropyrimidine-2,4(1H,3H)-dione (63 mg, 0.185 mmol, 66.0% yield). LCMS (high pH A): Rt=0.70 min, MH⁺=341.1

### Example CB44

### 1-(6-Fluoro-1-(piperidin-4-yl)-1H-indol-4-yl)dihydropyrimidine-2,4(1H,3H)-dione (ECB44)

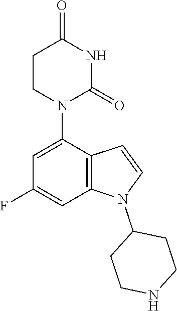 [SMILES: O=C1CCN(c2cc(F)cc3c2ccn3C2CCNCC2)C(=O)N1]

Benzyl 4-(4-(2,4-dioxotetrahydropyrimidin-1(2H)-yl)-6-fluoro-1H-indol-1-yl)piperidine-1-carboxylate (may be prepared as described in Description 318; 2.63 g, 5.66 mmol) was dissolved in a mixture of ethanol (40 mL) and ethyl acetate (10.00 mL), and Pd—C 10% (0.603 g, 0.566 mmol) and ammonium formate (1.785 g, 28.3 mmol) were added. The mixture was heated at reflux for 4 h, cooled and filtered through Celite. The celite pad was washed with DCM:ethanol (1:1, 1 L), and the filtrate was evaporated in vacuo to give 1-(6-fluoro-1-(piperidin-4-yl)-1H-indol-4-yl)dihydropyrimidine-2,4(1H,3H)-dione (1.78 g, 5.39 mmol, 95% yield). LCMS (high pH A): Rt=0.69 min, MH⁺=331.1

### Example CB45

### 1-(1-Methyl-1H-indol-4-yl)dihydropyrimidine-2,4(1H,3H)-dione (ECB45)

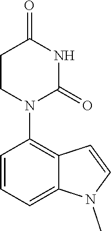 [SMILES: Cn1ccc2c(N3CCC(=O)NC3=O)cccc21]

A mixture of the 1-methyl-1H-indol-4-amine (58 mg, 0.4 mmol) in toluene (0.75 mL) and acrylic acid (0.082 mL, 1.20 mmol) was stirred in a sealed vessel at 45° C. for 6 h. Acetic acid (1.0 mL) and urea (0.108 g, 1.80 mmol) were added, and the reaction was heated to 130° C. After 15 h, the reaction was allowed to cool to room temperature, diluted with 2 M Na₂CO₃ (aq) (1 mL) and extracted with DCM (3×1 mL). The organic extracts were combined, passed through a hydrophobic frit, evaporated under a stream of nitrogen and purified by MDAP (high pH) to give 1-(1-methyl-1H-indol-4-yl)dihydropyrimidine-2,4(1H,3H)-dione (25 mg, 0.103 mmol, 23.12% yield). LCMS (high pH A): Rt=0.71 min, MH⁺=244

### Example CB46

### 1-(1-Methyl-1H-indazol-4-yl)dihydropyrimidine-2,4(1H,3H)-dione (ECB46)

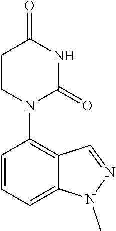 [SMILES: Cn1ncc2c(N3CCC(=O)NC3=O)cccc21]

1-Methyl-1H-indazol-4-amine (74 mg, 0.500 mmol) was suspended in toluene (0.50 mL) and treated with acrylic acid (0.103 mL, 1.500 mmol). The mixture was stirred in a stoppered vessel at 70° C. for 6 h. The reaction was allowed to cool to rt and treated with urea (0.135 g, 2.250 mmol) and acetic acid (0.75 mL). The mixture was stirred in a stoppered vessel at 125° C. for 15 h, cooled to room temperature, diluted with CHCl₃ (1 mL) and washed with 2 M Na₂CO₃ (aqueous). The organic layer was passed through a hydrophobic frit. The aqueous layer was extracted with CHCl₃ (2×1 mL), and the organic extracts were passed through a hydrophobic frit. The filtrates were under a stream of nitrogen and purified by MDAP (high pH) to give 1-(1-methyl-1H-indazol-4-yl)dihydropyrimidine-2,4(1H,3H)-dione (33 mg, 0.135 mmol, 25.7% yield). LCMS (high pH A): Rt=0.59 min, MH⁺=245

### Example CB47

### 1-(1-Methyl-1H-pyrrolo[3,2-c]pyridin-4-yl)dihydropyrimidine-2,4(1H,3H)-dione (ECB47)

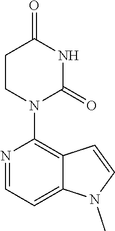 [SMILES: Cn1ccc2c(N3CCC(=O)NC3=O)nccc21]

A mixture of copper(I) iodide (7.77 mg, 0.041 mmol), potassium carbonate (113 mg, 0.815 mmol), 3-(2,4,6-trimethoxybenzyl)dihydropyrimidine-2,4(1H,3H)-dione (may be prepared as described in Description 329; 120 mg, 0.408 mmol), 4-bromo-1-methyl-1H-pyrrolo[3,2-c]pyridine (may be prepared as described in Description 330; 112 mg, 0.530 mmol), trans-N,N′-dimethylcyclohexane-1,2-diamine (0.013 mL, 0.082 mmol) and anhydrous 1,4-dioxane (2 mL) in a sealed vessel was evacuated and purged with nitrogen (×3). The mixture was stirred at 140° C. for 3 h and filtered through a C18 cartridge (1 g) preconditioned with MeCN (4 mL). The filtrate was evaporated and treated with trifluoroacetic acid (0.80 mL, 10.38 mmol) and Tf-OH (0.130 mL, 1.464 mmol). After 16 h, the reaction was heated to 80° C. for 8 h, evaporated under a stream of nitrogen and purified by MDAP (high pH) to give 1-(1-methyl-1H-pyrrolo[3,2-c]pyridin-4-yl)dihydropyrimidine-2,4(1H,3H)-dione (45 mg, 0.184 mmol, 45.2%). LCMS (high pH A): Rt=0.55 min, MH⁺=245

### Example CB48

### 1-(1-(2-Methoxyethyl)-1H-indol-4-yl)dihydropyrimidine-2,4(1H,3H)-dione (ECB48)

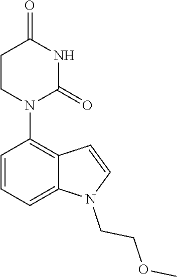 [SMILES: COCCn1ccc2c(N3CCC(=O)NC3=O)cccc21]

An 8 mL microwave vial was charged with 3,3′-((1-(2-methoxyethyl)-1H-indol-4-yl) azanediyl)dipropionic acid (may be prepared as described in Description 333; 240 mg, 0.718 mmol), urea (43.1 mg, 0.718 mmol) and acetic acid (3 mL, 52.4 mmol). The reaction vessel was sealed and microwaved to 145° C. for 1 h, concentrated and purified over a RediSep C-18 column (30 g), eluting with MeCN in H₂O (0.1% formic acid) to give crude product. This material was taken up in water (0.5 mL), stirred for 5 min and filtered to give 1-(1-(2-methoxyethyl)-1H-indol-4-yl)dihydropyrimidine-2,4(1H,3H)-dione (15 mg, 0.052 mmol, 7.20%). LCMS (high pH A): Rt=0.633 min, MH⁺=288.2

### Example CB49

### 1-(1,7-Dimethyl-1H-indol-4-yl)dihydropyrimidine-2,4(1H,3H)-dione (ECB49)

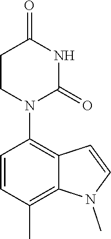 [SMILES: Cc1ccc(N2CCC(=O)NC2=O)c2ccn(C)c12]

1,7-Dimethyl-1H-indol-4-amine (may be prepared as described in Description 337; 250 mg, 1.560 mmol) and acrylic acid (214 μl, 3.12 mmol) were stirred for 3 h, then placed in a 10 mL microwave vial. Acetic acid (5 mL) and urea (199 mg, 3.31 mmol) were added, and the reaction vessel was sealed and heated to 140° C. for 1 hr. The reaction was concentrated and purified over a RediSep C-18 column (40 g), eluting with MeCN in H₂O (0.1% formic acid) to give 1-(1,7-dimethyl-1H-indol-4-yl)dihydropyrimidine-2,4(1H,3H)-dione (125 mg, 0.486 mmol, 31.2% yield). LCMS (formic A): Rt=0.68 min, MH⁺=258.2

### Example CB50

### 1-(1-Ethyl-1H-indol-4-yl)dihydropyrimidine-2,4(1H,3H)-dione (ECB50)

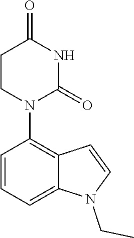 [SMILES: CCn1ccc2c(N3CCC(=O)NC3=O)cccc21]

1-Ethyl-1H-indol-4-amine (may be prepared as described in Description 339; 695 mg, 4.34 mmol) and acrylic acid (595 μl, 8.68 mmol) were stirred at 27° C. for 3 h and concentrated. Forty percent of this material was placed in a 10 mL microwave vial. Acetic acid (3 mL) and urea (39.5 mg, 0.657 mmol) were added, and the reaction vessel was sealed and heated to 145° C. for 1 hr. The reaction was concentrated and purified over a RediSep C-18 column (30 g), eluting with MeCN in H₂O (0.1% formic acid) to give 1-(1-ethyl-1H-indol-4-yl)dihydropyrimidine-2,4(1H,3H)-dione (7.5 mg, 0.029 mmol, 1.68% yield). LCMS (formic A): Rt=0.698 min, MH⁺=258.2

### Example CB51

### 1-(1-Ethyl-1H-pyrrolo[2,3-c]pyridin-4-yl)dihydropyrimidine-2,4(1H,3H)-dione (ECB51)

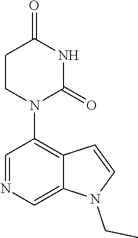 [SMILES: CCn1ccc2c(N3CCC(=O)NC3=O)cncc21]

To 1-(1-ethyl-1H-pyrrolo[2,3-c]pyridin-4-yl)-3-((2-(trimethylsilyl)ethoxy)methyl)dihydropyrimidine-2,4(1H,3H)-dione (may be prepared as described in Description 341; 500 mg, 1.287 mmol) in 1,4-dioxane (2.5 mL) was added HCl (4M in Dioxane) (2.5 ml, 82 mmol) in 1,4-dioxane (2.5 mL). The reaction was stirred at 25° C. for 3 h. Solvents were removed under reduced pressure and the residue was taken up in 1,4-dioxane (2.500 ml). HCl (4M in Dioxane) (6 mL, 197 mmol) was added, and the reaction was stirred for 3 h, concentrated and purified using reverse-phase chromatography, eluting with 5-100% MeCN in 0.1% ammonium carbonate (aqueous) to give 1-(1-ethyl-1H-pyrrolo[2,3-c]pyridin-4-yl)dihydropyrimidine-2,4(1H,3H)-dione (100 mg, 0.386 mmol, 30.0% yield). LCMS (high pH B): Rt=1.32 min, MH⁺=259.1

### Example CB52

### (S)-1-(1-(Morpholin-2-ylmethyl)-1H-indol-4-yl)dihydropyrimidine-2,4(1H,3H)-dione hydrochloride (ECB52)

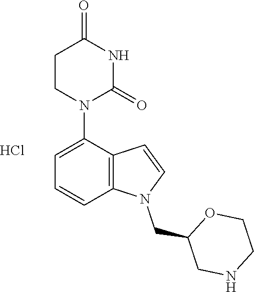 [SMILES: Cl ; O=C1CCN(c2cccc3c2ccn3C[C@@H]2CNCCO2)C(=O)N1]

To a solution of tert-butyl (R)-2-((4-(2,4-dioxo-3-((2-(trimethylsilyl)ethoxy)methyl)tetrahydropyrimidin-1(2H)-yl)-1H-indol-1-yl)methyl) morpholine-4-carboxylate (may be prepared as described in Description 358; 6.20 g, 11.1 mmol) in DCM (50.0 mL) was added TFA (15.0 mL, 195 mmol). The reaction mixture was stirred at room temperature for 1 h. The reaction mixture was concentrated under reduced pressure and sequentially washed with petroleum ether and toluene. The sample was dissolved in methanol (50 mL) and treated with ammonia (7N in MeOH, 10.0 mL, 462 mmol) and the mixture was stirred at room temperature for 1 h. The sample was purified by preparative HPLC (X-Select C18 20 mm×150 mm) using a 0-40% acetonitrile-water (+0.1% HCl) gradient over 14 min (flow rate 20 ml/min). The appropriate fractions were combined and lyophilised to give the desired product (1.60 g, 39% yield) as a pale pink solid. LCMS (formic B): Rt=0.99 min, MH⁺=329.

### Example CB53

### (R)-1-(1-(Morpholin-2-ylmethyl)-1H-indol-4-yl)dihydropyrimidine-2,4(1H,3H)-dione hydrochloride (ECB53)

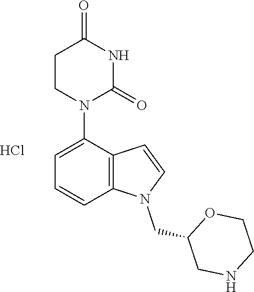 [SMILES: Cl ; O=C1CCN(c2cccc3c2ccn3C[C@H]2CNCCO2)C(=O)N1]

To a solution of tert-butyl(S)-2-((4-(2,4-dioxo-3-((2-(trimethylsilyl)ethoxy)methyl)tetrahydropyrimidin-1(2H)-yl)-1H-indol-1-yl)methyl) morpholine-4-carboxylate (may be prepared as described in Description 361; 2.60 g, 4.65 mmol) in DCM (25 mL) at 0° C. was added TFA (10.0 mL, 130 mmol), then the mixture was stirred at room temperature for 1 h. The reaction mixture was concentrated under reduced pressure and washed with petroleum ether and toluene. The sample was dissolved in methanol (25 mL) and ammonia (7N in methanol, 0.101 mL, 4.65 mmol) was added mixture stirred at room temperature for 1 h. The reaction mixture was concentrated under reduced pressure and washed with petroleum ether. The sample was purified by preparative HPLC (X-Select C18 20 mm×150 mm) using a 0-40% acetonitrile-water (+0.1% HCl) gradient over 12 min (flow rate 12 mL/min). The appropriate fractions were combined and lyophilised to give the desired product (0.861 g, 55% yield) as an off white solid. LCMS (formic B): Rt=0.99 min, MH⁺=329.

### Degron+Linker Examples

The following compounds are compounds of formula (I), (Ia) or (Ib) where p is 1 and q is 0. It should be noted that the compounds described in Descriptions 5, 9, 26, 29-31 36-41, 189, 245, 292 and 318 could also be considered to be compounds of formula (I), (Ia) or (Ib) where p is 1 and q is 0.

### Example CBL1

### 1-(1-(1-(azetidin-3-ylmethyl)piperidin-4-yl)-1H-indol-4-yl)dihydropyrimidine-2,4(1H,3H)-dione (ECBL1)

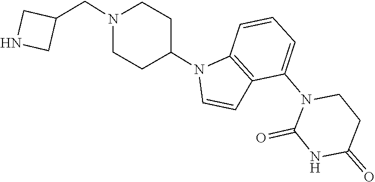 [SMILES: O=C1CCN(c2cccc3c2ccn3C2CCN(CC3CNC3)CC2)C(=O)N1]

tert-Butyl 3-formylazetidine-1-carboxylate (0.020 g, 0.110 mmol), 1-(1-(piperidin-4-yl)-1H-indol-4-yl)dihydropyrimidine-2,4(1H,3H)-dione (may be prepared as described in Example CB1; 0.031 g, 0.1 mmol) and sodium triacetoxyborohydride (0.106 g, 0.500 mmol) dissolved in THF (0.5 mL) was left to stir at 20° C. for 114 h. The solvent was dried down and redissolved in DCM (1 mL) and water (1 mL) was added. The organic phase was pipetted out and filtered through a hydrophobic column. The aqueous phase was washed again with DCM (2 mL) and the organic phase was filtered through a hydrophobic column. The solvent was removed under a stream of nitrogen to give the crude product, DCM (500 μL) was added, followed by TFA (500 μL). After 2 h, the solvent was dried under a stream of nitrogen to give the crude product as a TFA salt. This was dissolved in DCM (1.5 mL) and then passed through pre-conditioned Agilent StratoSpheres PL-HCO3 MP SPE cartridge (200 mg, MeOH 1 mL). The cartridge was then washed with additional MeOH (1.5 mL), and the solvent was removed under a stream of nitrogen to give the title compound which was carried through to the next reaction. LCMS (formic A): Rt=0.65 min, MH⁺=382

### Example CBL2

### (R)-1-(1-(1-(piperidin-3-ylmethyl)piperidin-4-yl)-1H-indol-4-yl)dihydropyrimidine-2,4(1H,3H)-dione (ECBL2)

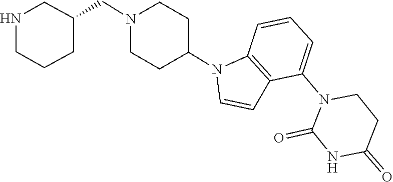 [SMILES: O=C1CCN(c2cccc3c2ccn3C2CCN(C[C@@H]3CCCNC3)CC2)C(=O)N1]

tert-Butyl (R)-3-formylpiperidine-1-carboxylate (0.023 g, 0.110 mmol), 1-(1-(piperidin-4-yl)-1H-indol-4-yl)dihydropyrimidine-2,4(1H,3H)-dione (may be prepared as described in Example CB1; 0.031 g, 0.1 mmol) and sodium triacetoxyborohydride (0.106 g, 0.500 mmol) dissolved in THF (0.5 mL) was left to stir at 20° C. for 114 h. The solvent was dried down and redissolved in DCM (1 mL) and water (1 mL) was added. The organic phase was pipetted out and filtered through a hydrophobic column. The aqueous phase was washed again with DCM (2 mL) and the organic phase was filtered through a hydrophobic column. The solvent was removed under a stream of nitrogen to give the crude product, DCM (500 μL) was added, followed by TFA (500 μL). After 2 h, the solvent was dried under a stream of nitrogen to give the crude product as a TFA salt. This was dissolved in DCM (1.5 mL) and then passed through pre-conditioned Agilent StratoSpheres PL-HCO3 MP SPE cartridge (200 mg, MeOH 1 mL). The cartridge was then washed with additional MeOH 1.5 mL, and the solvent was removed under a stream of nitrogen to give the title compound which was carried through to the next reaction. LCMS (formic A): Rt=0.80 min, MH⁺=410.

### Example CBL3

### 1-(1-(1-((4-Fluoropiperidin-4-yl)methyl)piperidin-4-yl)-1H-pyrrolo[2,3-c]pyridin-4-yl)dihydropyrimidine-2,4(1H,3H)-dione hydrochloride (ECBL3)

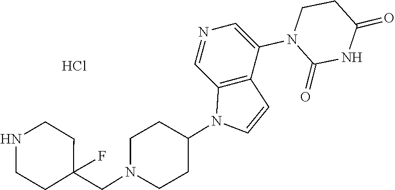 [SMILES: O=C1CCN(c2cncc3c2ccn3C2CCN(CC3(F)CCNCC3)CC2)C(=O)N1 ; Cl]

4 M Hydrogen chloride in dioxane (2 mL, 4 mmol) was added to a stirred solution of tert-butyl 4-((4-(4-(2,4-dioxotetrahydropyrimidin-1(2H)-yl)-1H-pyrrolo[2,3-c]pyridin-1-yl)piperidin-1-yl)methyl)-4-fluoropiperidine-1-carboxylate (may be prepared as described in Description 29; 90 mg, 0.170 mmol) in dioxane (1 mL). The reaction mixture was stirred at room temperature for 1.5 h. Diethyl ether (25 mL) was added and the mixture was stirred for 10 min. The solvent was deacanted off. The residue was triturated with diethyl ether to give the title compound as a colourless solid (74 mg, 0.159 mmol, 93% yield). LCMS (high pH A): Rt=0.66 min, MH⁺=429.

### Example CBL4

### 2-(4-(4-(2,4-Dioxotetrahydropyrimidin-1(2H)-yl)-1H-indol-1-yl)piperidin-1-yl) acetic acid trifluoroacetate (ECBL4)

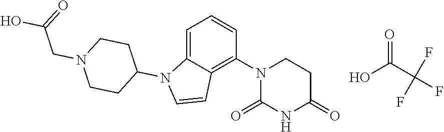 [SMILES: O=C(O)CN1CCC(n2ccc3c(N4CCC(=O)NC4=O)cccc32)CC1 ; O=C(O)C(F)(F)F]

A solution of tert-butyl 2-(4-(4-(2,4-dioxotetrahydropyrimidin-1(2H)-yl)-1H-indol-1-yl)piperidin-1-yl)acetate (may be prepared as described in Description 30; 430 mg, 1.008 mmol) in DCM (10 mL) was treated with TFA (500 μl, 6.49 mmol) and left to stir at room temperature for 4 h in a stoppered vessel. The mixture was then heated to 45° C. and stirred for 16 h. The reaction mixture was then evaporated under a stream of nitrogen and dried in a vacuum oven at 40° C. This yielded the title compound as an off white powder (666 mg, 1.375 mmol) and was used without purification in the next step. LCMS (high pH A): Rt=0.52 min, MH⁺=371.

### Example CBL5

### 4-(4-(4-(2,4-Dioxotetrahydropyrimidin-1(2H)-yl)-1H-indol-1-yl)piperidin-1-yl)benzoic acid trifluoroacetate (ECBL5)

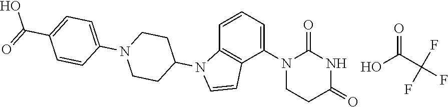 [SMILES: O=C1CCN(c2cccc3c2ccn3C2CCN(c3ccc(C(=O)O)cc3)CC2)C(=O)N1 ; O=C(O)C(F)(F)F]

tert-Butyl 4-(4-(4-(2,4-dioxotetrahydropyrimidin-1(2H)-yl)-1H-indol-1-yl)piperidin-1-yl)benzoate (may be prepared as described in Description 31; 61 mg, 0.125 mmol) was dissolved in 1,4-Dioxane (0.5 mL). 4 M HCl in dioxane (0.030 mL, 0.987 mmol) was added, and solution was left at room temperature for 16 h. Solvent was then removed via rotary evaporation and the resulting solid was dissolved in anhydrous DCM (0.500 mL) before TFA (0.1 mL, 1.298 mmol) was added. The mixture was left for 64 h at room temperature before being heated and stirred at 40° C. for 4 h. Additional TFA (0.1 mL, 1.298 mmol) was added, and the mixture was left to stir at 40° C. for 1.5 h. Reaction mixture was then evaporated under a stream of nitrogen to give the title compound as a purple powder (54 mg, 0.099 mmol, 79% yield). LCMS (high pH A): Rt=0.61 min, MH⁺=433

### Example CBL6

### 1-(1-(1-((4-Fluoropiperidin-4-yl)methyl)piperidin-4-yl)-1H-indazol-4-yl)dihydropyrimidine-2,4(1H,3H)-dione hydrochloride (ECBL6)

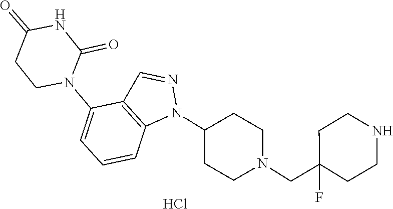 [SMILES: O=C1CCN(c2cccc3c2cnn3C2CCN(CC3(F)CCNCC3)CC2)C(=O)N1 ; Cl]

A mixture of 1-(1-(piperidin-4-yl)-1H-indazol-4-yl)dihydropyrimidine-2,4(1H,3H)-dione, trifluoroacetic acid salt (may be prepared as described in Example ECB4; 214 mg, 0.500 mmol) and tert-butyl 4-fluoro-4-formylpiperidine-1-carboxylate (may be prepared as described in Description D28; 231 mg, 1.000 mmol) in dichloromethane (5 mL) was stirred at room temperature for 1 hour. Triethylamine (101 mg, 0.139 mL, 1.000 mmol) was added, followed by sodium triacetoxyborohydride (318 mg, 1.500 mmol). The reaction mixture was stirred at room temperature for 1 hour then allowed to stand for 16 hours. Saturated sodium bicarbonate solution (10 mL) was added, and the mixture was stirred for 10 minutes. The organic phase was separated, and the aqueous phase was extracted with dichloromethane (2×5 mL). The combined organics were dried and evaporated. The residue was chromatographed [0-20% ethanol in ethyl acetate] to give intermediate tert-butyl 4-((4-(4-(2,4-dioxotetrahydropyrimidin-1(2H)-yl)-1H-indazol-1-yl)piperidin-1-yl)methyl)-4-fluoropiperidine-1-carboxylate, which was taken up in dioxane (2 mL). 4M Hydrogen chloride in dioxane (4 mL, 8 mmol) was added, the reaction was stirred at room temperature for 2 hours. Diethyl ether (50 mL) was added, and the mixture was stirred at room temperature for 15 minutes. The supernatant liquid was decanted off, and the residue was dried to give 1-(1-(1-((4-fluoropiperidin-4-yl)methyl)piperidin-4-yl)-1H-indazol-4-yl)dihydropyrimidine-2,4(1H,3H)-dione hydrochloride (86 mg, 0.185 mmol, 93% yield). LCMS (high pH A): Rt=0.78 min, MH⁺=429.1

### Example CBL7

### 1-(1-(1-((4-Fluoropiperidin-4-yl)methyl)piperidin-4-yl)-1H-pyrazolo[3,4-c]pyridin-4-yl)dihydropyrimidine-2,4(1H,3H)-dione hydrochloride (ECBL7)

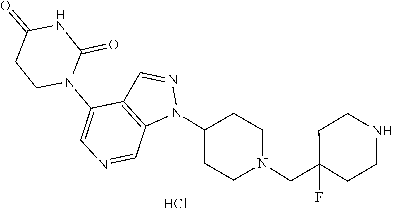 [SMILES: O=C1CCN(c2cncc3c2cnn3C2CCN(CC3(F)CCNCC3)CC2)C(=O)N1 ; Cl]

A mixture of 1-(1-(piperidin-4-yl)-1H-pyrazolo[3,4-c]pyridin-4-yl)dihydropyrimidine-2,4(1H,3H)-dione (may be prepared as described in Example ECB8; 150 mg, 0.477 mmol) and tert-butyl 4-fluoro-4-formylpiperidine-1-carboxylate (may be prepared as described in Description D28; 221 mg, 0.954 mmol) in dichloromethane (5 mL) was stirred at room temperature for 1 hour. Triethylamine (101 mg, 0.133 mL, 0.954 mmol) was added, followed by sodium triacetoxyborohydride (303 mg, 0.954 mmol).

The reaction mixture was stirred at room temperature for 1 hour then allowed to stand for 16 hours. Saturated sodium bicarbonate solution (10 mL) was added, and the mixture was stirred for 10 minutes. The organic phase was separated, and the aqueous phase was extracted with dichloromethane (2×5 mL). The combined organics were dried and evaporated. The residue was chromatographed [0-20% ethanol in ethyl acetate] to give intermediate tert-butyl 4-((4-(4-(2,4-dioxotetrahydropyrimidin-1(2H)-yl)-1H-pyrazolo[3,4-c]pyridin-1-yl)piperidin-1-yl)methyl)-4-fluoropiperidine-1-carboxylate, which was taken up in dioxane (4 mL). 4M Hydrogen chloride in dioxane (4 mL, 8 mmol) was added, the reaction was stirred at room temperature for 2 hours. Diethyl ether (50 mL) was added, and the mixture was stirred at room temperature for 15 minutes. The supernatant liquid was decanted off, and the residue was dried to give 1-(1-(1-((4-fluoropiperidin-4-yl)methyl)piperidin-4-yl)-1H-pyrazolo[3,4-c]pyridin-4-yl)dihydropyrimidine-2,4(1H,3H)-dione hydrochloride (94 mg, 0.202 mmol, 89% yield). LCMS (high pH A): Rt=0.65 min, MH⁺=430.1

### Examples CBL8-CBL11

Four microwave vials containing one of the aldehyde monomers from the table below (0.220 mmol), 1-(1-(piperidin-4-yl)-1H-indazol-4-yl)dihydropyrimidine-2,4(1H,3H)-dione, trifluoroacetic acid salt (may be prepared as described in Example ECB4; 0.085 g, 0.2 mmol), picoline-borane complex (0.032 g, 0.300 mmol) and acetic acid (0.034 mL, 0.600 mmol) dissolved in methanol (1 mL) were stirred at room temperature for 18 h. The solvent was dried under a stream of nitrogen to give the crude products. (Reactions which did not go to completion were re-treated with aldehyde, borane and AcCO₂H). The samples were dissolved in 1:1 methanol:DMSO (1 mL) and purified by MDAP (formic). Each product was dissolved in dioxane (600 μl) and treated with 4M HCl in dioxane (400 μl). After 1 hour, reactions were concentrated under a stream of nitrogen. All reactions were incomplete and redissolved in dioxane (600 μl) and treated with 4M HCl in dioxane (300 μl). After 1 hour, reactions were concentrated under a stream of nitrogen to obtain the following compounds:

| | Product structure and name | Aldehyde | Mass, |
|---|---|---|---|
| Example | Analytical | Monomer | yield |
| CBL8 | 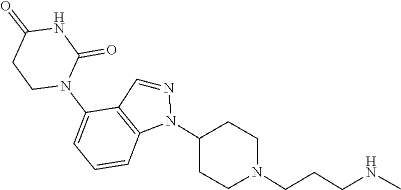 [SMILES: CNCCCN1CCC(n2ncc3c(N4CCC(=O)NC4=O)cccc32)CC1] | tert-Butyl methyl(3- oxopropyl) carbamate | 64.4 mg, 75% |
| | 1-(1-(1-(3-(Methylamino)propyl)piperidin-4-yl)-1H-indazol- | | |
| | 4-yl)dihydropyrimidine-2,4(1H,3H)-dione | | |
| | LCMS (formic A): Rt = 0.69 min, MH⁺ = 385 | | |
| CBL9 | 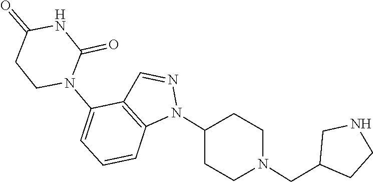 [SMILES: O=C1CCN(c2cccc3c2cnn3C2CCN(CC3CCNC3)CC2)C(=O)N1] | tert-butyl 3- formyl- pyrrolidine-1- carboxylate | 72.8 mg, 83% |
| | 1-(1-(1-(Pyrrolidin-3-ylmethyl)piperidin-4-yl)-1H-indazol-4- | | |
| | yl)dihydropyrimidine-2,4(1H,3H)-dione | | |
| | LCMS (formic A): Rt = 0.68 min, MH⁺ = 397 | | |
| CBL10 | 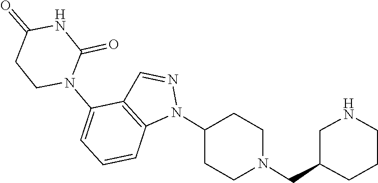 [SMILES: O=C1CCN(c2cccc3c2cnn3C2CCN(C[C@@H]3CCCNC3)CC2)C(=O)N1] | tert-butyl (R)-3- formyl- piperidine-1- carboxylate | 77.5 mg, 85% |
| | (R)-1-(1-(1-(Piperidin-3-ylmethyl)piperidin-4-yl)-1H- | | |
| | indazol-4-yl)dihydropyrimidine-2,4(1H,3H)-dione | | |
| | LCMS (formic A): Rt = 0.77 min, MH⁺ = 411 | | |
| CBL11 | 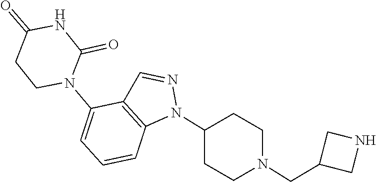 [SMILES: O=C1CCN(c2cccc3c2cnn3C2CCN(CC3CNC3)CC2)C(=O)N1] | tert-butyl 3- formyl- azetidine-1- carboxylate | 78.3 mg, 55% |
| | 1-(1-(1-(azetidin-3-ylmethyl)piperidin-4-yl)-1H-indazol-4- | | |
| | yl)dihydropyrimidine-2,4(1H,3H)-dione | | |
| | LCMS (formic A): Rt = 0.62 min, MH⁺ = 383 | | |

### Example CBL12

### 1-(1-(1-(Piperidin-4-ylmethyl)piperidin-4-yl)-1H-indol-4-yl)dihydropyrimidine-2,4(1H,3H)-dione hydrochloride (ECBL12)

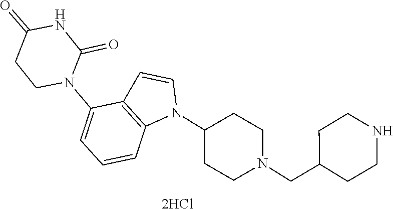 [SMILES: O=C1CCN(c2cccc3c2ccn3C2CCN(CC3CCNCC3)CC2)C(=O)N1 ; "2HCl"]

A solution of tert-butyl 4-((4-(4-(2,4-dioxotetrahydropyrimidin-1(2H)-yl)-1H-indol-1-yl)piperidin-1-yl)methyl)piperidine-1-carboxylate (may be prepared as described in Description 244; 774 mg, 1.519 mmol) in DCM (20 mL) was treated with 4 M HCl in 1,4-dioxane (5.0 mL, 20.00 mmol). The reaction mixture was allowed to stir at room temperature in a loosely stoppered vessel for 2 h (a precipitate formed over this time). The reaction mixture was filtered, the solid washed with diethyl ether (20 mL) and dried in a vacuum oven to give 1-(1-(1-(piperidin-4-ylmethyl)piperidin-4-yl)-1H-indol-4-yl)dihydropyrimidine-2,4(1H,3H)-dione hydrochloride (448 mg, 0.929 mmol). LCMS (high pH A): Rt=0.82 min, MH⁺=410

### Examples CBL13-CBL23

To 1-(1-(piperidin-4-yl)-1H-indazol-4-yl)dihydropyrimidine-2,4(1H,3H)-dione 2,2,2-trifluoroacetate (may be prepared as described in Example ECB4; 1.539 g, 3.60 mmol) in methanol (24 mL) was added DIPEA (624 μL, 3.60 mmol). After 30 minutes solvent was removed under a stream of nitrogen for 5 h. The amine was redissolved in methanol (24 mL), and 1 mL aliquots of this solution were added to a vial equipped with a stirrer bar and containing one of the aldehyde monomers from the table below (0.20 mmol), acetic acid (0.026 mL, 0.45 mmol) and 2-methylpyridine-borane complex (0.024 g, 0.225 mmol). The reactions were stirred at room temperature for 22 h (reactions that did not go to completion were heated to 50° C. and stirred for a further 3 h). The reactions were blown down using a stream of nitrogen at 40° C. then redissolved in DMSO (0.8 mL), using sonication to promote dissolution. The samples were filtered, then purified by MDAP (formic), and the pure fractions were blown down using a stream of nitrogen at 40° C. For BOC-deprotection, 1,4-dioxane (0.6 mL) was added to each of the vials of purified reductive amination product, followed by 300 μL of 4 M HCl in dioxane solution. The vials were sealed and stirred at room temperature for 1 h, placed in the freezer for 16 h, allowed to stand at room temperature for 4 h and were blown down under a stream of nitrogen for 3 h at 40° C. to give the following crude compounds:

| | Product structure and name | |
|---|---|---|
| Example | Analytical | Aldehyde Monomer |
| CBL13 | 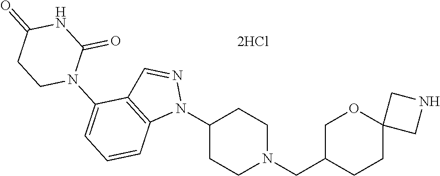 [SMILES: O=C1CCN(c2cccc3c2cnn3C2CCN(CC3CCC4(CNC4)OC3)CC2)C(=O)N1 ; "2HCl"] | tert-butyl 7-formyl- 5-oxa-2- azaspiro[3.5]nonane- 2-carboxylate |
| | 1-(1-(1-((5-oxa-2-azaspiro[3.5]nonan-7-yl)methyl)piperidin- | |
| | 4-yl)-1H-indazol-4-yl)dihydropyrimidine-2,4(1H,3H)-dione | |
| | dihydrochloride | |
| | LCMS (high pH A): Rt = 0.70 min, MH⁺ = 453.2 | |
| CBL14 | 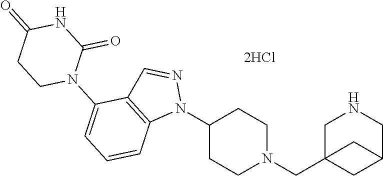 [SMILES: O=C1CCN(c2cccc3c2cnn3C2CCN(CC34CNCC(C3)C4)CC2)C(=O)N1 ; "2HCl"] | tert-butyl 1-formyl- 3- azabicyclo[3.1.1] heptane-3-carboxylate |
| | 1-(1-(1-((3-azabicyclo[3.1.1]heptan-1-yl)methyl)piperidin-4- | |
| | yl)-1H-indazol-4-yl)dihydropyrimidine-2,4(1H,3H)-dione | |
| | dihydrochloride | |
| | LCMS (high pH A): Rt = 0.75 min, MH⁺ = 423.2 | |
| CBL15 | 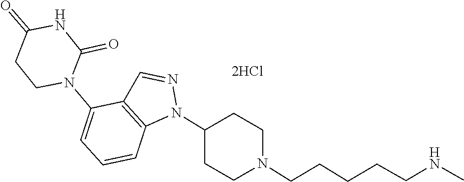 [SMILES: CNCCCCCN1CCC(n2ncc3c(N4CCC(=O)NC4=O)cccc32)CC1] | tert-butyl methyl(5- oxopentyl)carbamate |
| | 1-(1-(1-(5-(methylamino)pentyl)piperidin-4-yl)-1H-indazol- | |
| | 4-yl)dihydropyrimidine-2,4(1H,3H)-dione dihydrochloride | |
| | LCMS (high pH A): Rt = 0.77 min, MH⁺ = 413.5 | |
| CBL16 | 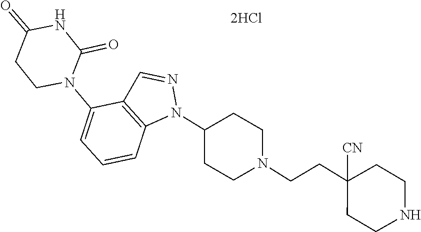 [SMILES: N#CC1(CCN2CCC(n3ncc4c(N5CCC(=O)NC5=O)cccc43)CC2)CCNCC1] | tert-butyl 4-cyano-4- (2- oxoethyl)piperidine- 1-carboxylate |
| | 4-(2-(4-(4-(2,4-dioxotetrahydropyrimidin-1(2H)-yl)-1H- | |
| | indazol-1-yl)piperidin-1-yl)ethyl)piperidine-4-carbonitrile | |
| | dihydrochloride | |
| | LCMS (high pH A): Rt = 0.73 min, MH⁺ = 450.1 | |
| CBL17 | 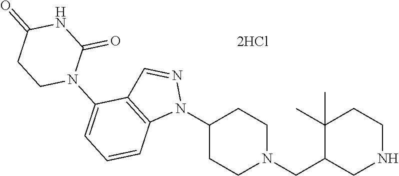 [SMILES: CC1(C)CCNCC1CN1CCC(n2ncc3c(N4CCC(=O)NC4=O)cccc32)CC1 ; "2HCl"] | tert-butyl 3-formyl- 4,4- dimethylpiperidine- 1-carboxylate |
| | 1-(1-(1-((4,4-dimethylpiperidin-3-yl)methyl)piperidin-4-yl)- | |
| | 1H-indazol-4-yl)dihydropyrimidine-2,4(1H,3H)-dione | |
| | dihydrochloride | |
| | LCMS (high pH A): Rt = 0.89 min, MH⁺ = 439.1 | |
| CBL18 | 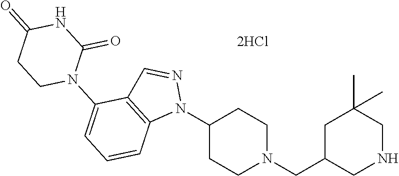 [SMILES: CC1(C)CNCC(CN2CCC(n3ncc4c(N5CCC(=O)NC5=O)cccc43)CC2)C1 ; "2HCl"] | tert-butyl 3-formyl- 5,5- dimethylpiperidine- 1-carboxylate |
| | 1-(1-(1-((5,5-dimethylpiperidin-3-yl)methyl)piperidin-4-yl)- | |
| | 1H-indazol-4-yl)dihydropyrimidine-2,4(1H,3H)-dione | |
| | dihydrochloride | |
| | LCMS (high pH A): Rt = 0.90 min, MH⁺ = 439.1 | |
| CBL19 | 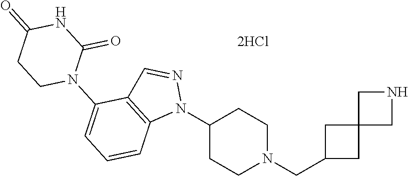 [SMILES: O=C1CCN(c2cccc3c2cnn3C2CCN(CC3CC4(CNC4)C3)CC2)C(=O)N1 ; "2HCl"] | tert-butyl 6-formyl- 2- azaspiro[3.3]heptane- 2-carboxylate |
| | 1-(1-(1-((5,5-dimethylpiperidin-3-yl)methyl)piperidin-4-yl)- | |
| | 1H-indazol-4-yl)dihydropyrimidine-2,4(1H,3H)-dione | |
| | dihydrochloride | |
| | LCMS (high pH A): Rt = 0.71 min, MH⁺ = 423.2 | |
| CBL20 | 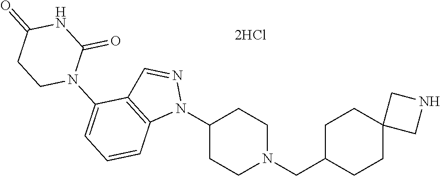 [SMILES: O=C1CCN(c2cccc3c2cnn3C2CCN(CC3CCC4(CC3)CNC4)CC2)C(=O)N1 ; "2HCl"] | tert-butyl 7-formyl- 2- azaspiro[3.5]nonane- 2-carboxylate |
| | 1-(1-(1-((2-azaspiro[3.5]nonan-7-yl)methyl)piperidin-4-yl)- | |
| | 1H-indazol-4-yl)dihydropyrimidine-2,4(1H,3H)-dione | |
| | dihydrochloride | |
| | LCMS (high pH A): Rt = 0.80 min, MH⁺ = 451.3 | |
| CBL21 | 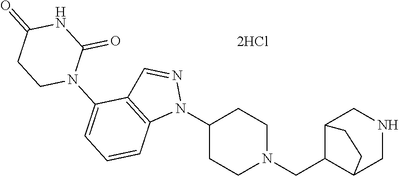 [SMILES: O=C1CCN(c2cccc3c2cnn3C2CCN(CC3C4CCC3CNC4)CC2)C(=O)N1 ; "2HCl"] | tert-butyl 8-formyl- 3- azabicyclo[3.2.1] octane-3-carboxylate |
| | 1-(1-(1-((3-azabicyclo[3.2.1]octan-8-yl)methyl)piperidin-4- | |
| | yl)-1H-indazol-4- | |
| | yl)dihydropyrimidine-2,4(1H,3H)-dione dihydrochloride | |
| | LCMS (high pH A): Rt = 0.78 min, MH⁺ = 437.2 | |
| CBL22 | 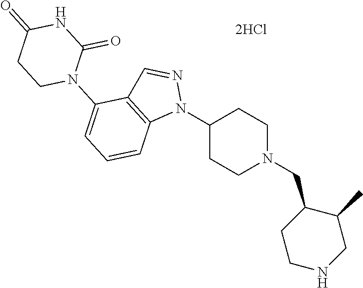 [SMILES: C[C@H]1CNCC[C@H]1CN1CCC(n2ncc3c(N4CCC(=O)NC4=O)cccc32)CC1 ; "2HCl"] | tert-butyl (3R,4R)-4- formyl-3- methylpiperidine-1- carboxylate |
| | 1-(1-(1-(((3R,4R)-3-methylpiperidin-4-yl)methyl)piperidin-4- | |
| | yl)-1H-indazol-4- | |
| | yl)dihydropyrimidine-2,4(1H,3H)-dione dihydrochloride | |
| | LCMS (high pH A): Rt = 0.77 min, MH⁺ = 425.3 | |
| CBL23 | 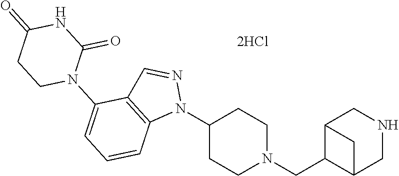 [SMILES: O=C1CCN(c2cccc3c2cnn3C2CCN(CC3C4CNCC3C4)CC2)C(=O)N1 ; "2HCl"] | tert-butyl 6-formyl- 3- azabicyclo[3.1.1] heptane-3-carboxylate |
| | 1-(1-(1-((3-azabicyclo[3.1.1]heptan-6-yl)methyl)piperidin-4- | |
| | yl)-1H-indazol-4-yl)dihydropyrimidine-2,4(1H,3H)-dione | |
| | dihydrochloride | |
| | LCMS (high pH A): Rt = 0.59 min, MH⁺ = 423.2 | |

### Examples CBL24-CBL39

To 1-(1-(piperidin-4-yl)-1H-indazol-4-yl)dihydropyrimidine-2,4(1H,3H)-dione 2,2,2-trifluoroacetate (may be prepared as described in Example ECB4; 1.603 g, 3.75 mmol) in methanol (24 mL) was added DIPEA (875 μL, 5.04 mmol). After 30 minutes the reaction was concentrated. The amine was redissolved in methanol (37.5 mL), and 1.5 mL aliquots of this solution were added to a vial equipped with a stirrer bar and containing one of the aldehyde monomers from the table below (0.18 mmol), acetic acid (0.026 mL, 0.45 mmol) and 2-methylpyridine-borane complex (0.024 g, 0.225 mmol). The reactions were stirred at 38° C. for 18 h. Reactions that did not go to completion were treated with additional acetic acid (0.026 mL, 0.45 mmol) and 2-methylpyridine-borane complex (0.024 g, 0.225 mmol), heated to 80° C. and stirred for a further 30 min. Reactions that were still not complete were heated to 90° C. for 1 h. The reactions were blown down using a stream of nitrogen, then purified by MDAP (formic) to give the BOC-protected products. For BOC-deprotection, 1,4-dioxane (0.3 mL) was added to each of the vials of purified reductive amination product, followed by 200 μL of 4 M HCl in dioxane solution. The vials were sealed and stirred at room temperature for 2 h, then were blown down under a stream of nitrogen to give the following compounds:

| | Product structure and name | | Mass, |
|---|---|---|---|
| Example | Analytical | Aldehyde Monomer | yield |
| CBL24 | 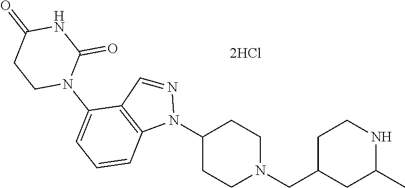 [SMILES: CC1CC(CN2CCC(n3ncc4c(N5CCC(=O)NC5=O)cccc43)CC2)CCN1 ; "2HCl"] | tert-butyl 4-formyl- 2- methylpiperidine- 1-carboxylate | 61.9 mg, 90% |
| | 1-(1-(1-((2-methylpiperidin-4-yl)methyl)piperidin-4- | | |
| | yl)-1H-indazol-4-yl)dihydropyrimidine-2,4(1H,3H)- | | |
| | dione dihydrochloride | | |
| | LCMS (high pH A): Rt = 0.73 min, MH⁺ = 425 | | |
| CBL25 | 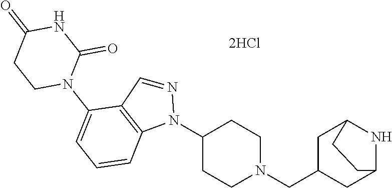 [SMILES: O=C1CCN(c2cccc3c2cnn3C2CCN(CC3CC4CCC(C3)N4)CC2)C(=O)N1 ; "2HCl"] | tert-butyl 3-formyl- 8- azabicyclo[3.2.1] octane-8- carboxylate | 63.8 mg, 90% |
| | 1-(1-(1-((8-azabicyclo[3.2.1]octan-3- | | |
| | yl)methyl)piperidin-4-yl)-1H-indazol-4- | | |
| | yl)dihydropyrimidine-2,4(1H,3H)-dione | | |
| | dihydrochloride | | |
| | LCMS (high pH A): Rt = 0.75 min, MH⁺ = 437 | | |
| CBL26 | 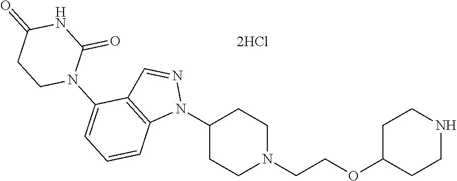 [SMILES: O=C1CCN(c2cccc3c2cnn3C2CCN(CCOC3CCNCC3)CC2)C(=O)N1 ; "2HCl"] | tert-butyl 4-(2- oxoethoxy)piper- idine-1-carboxylate | 56.1 mg, 78% |
| | 1-(1-(1-(2-(piperidin-4-yloxy)ethyl)piperidin-4-yl)- | | |
| | 1H-indazol-4-yl)dihydropyrimidine-2,4(1H,3H)-dione | | |
| | dihydrochloride | | |
| | LCMS (high pH A): Rt = 0.70 min, MH⁺ = 441 | | |
| CBL27 | 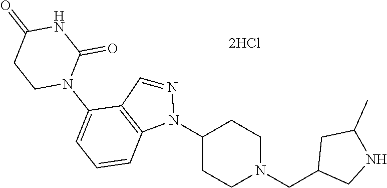 [SMILES: CC1CC(CN2CCC(n3ncc4c(N5CCC(=O)NC5=O)cccc43)CC2)CN1 ; "2HCl"] | tert-butyl 4-formyl- 2- methylpyrrolidine- 1-carboxylate | 49.5 mg, 73.8% |
| | 1-(1-(1-((5-methylpyrrolidin-3-yl)methyl)piperidin-4- | | |
| | yl)-1H-indazol-4-yl)dihydropyrimidine-2,4(1H,3H)- | | |
| | dione dihydrochloride | | |
| | LCMS (high pH A): Rt = 0.71 min, MH⁺ = 411 | | |
| CBL28 | 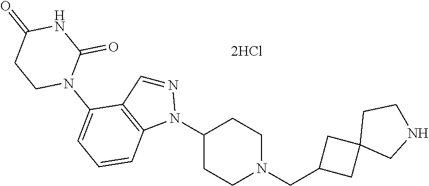 [SMILES: O=C1CCN(c2cccc3c2cnn3C2CCN(CC3CC4(CCNC4)C3)CC2)C(=O)N1 ; "2HCl"] | tert-butyl 2-formyl- 6- azaspiro[3.4]octane- 6-carboxylate | 59 mg, 83% |
| | 1-(1-(1-((6-azaspiro[3.4]octan-2- | | |
| | yl)methyl)piperidin-4-yl)-1H-indazol-4- | | |
| | yl)dihydropyrimidine-2,4(1H,3H)-dione | | |
| | dihydrochloride | | |
| | LCMS (high pH A): Rt = 0.59 min, MH⁺ = 409 | | |
| CBL29 | 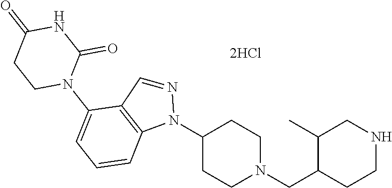 [SMILES: CC1CNCCC1CN1CCC(n2ncc3c(N4CCC(=O)NC4=O)cccc32)CC1 ; "2HCl"] | tert-butyl 4-formyl- 3- methylpiperidine- 1-carboxylate | 54.1 mg, 78% |
| | 1-(1-(1-((3-methylpiperidin-4-yl)methyl)piperidin-4- | | |
| | yl)-1H-indazol-4-yl)dihydropyrimidine-2,4(1H,3H)- | | |
| | dione dihydrochloride | | |
| | LCMS (high pH A): Rt = 0.77 min, MH⁺ = 425 | | |
| CBL30 | 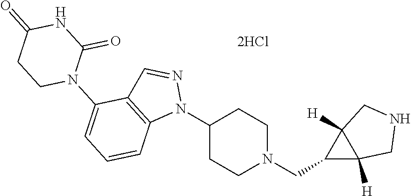 [SMILES: O=C1CCN(c2cccc3c2cnn3C2CCN(C[C@@H]3[C@@H]4CNC[C@@H]43)CC2)C(=O)N1 ; "2HCl"] | tert-butyl (1R,5S,6s)-6- formyl-3- azabicyclo[3.1.0] hexane-3- carboxylate | 49.1 mg, 73.6% |
| | 1-(1-(1-(((1R,5S,6s)-3-azabicyclo[3.1.0]hexan-6- | | |
| | yl)methyl)piperidin-4-yl)-1H-indazol-4- | | |
| | yl)dihydropyrimidine-2,4(1H,3H)-dione | | |
| | dihydrochloride | | |
| | LCMS (high pH A): Rt = 0.59 min, MH⁺ = 409 | | |
| CBL31 | 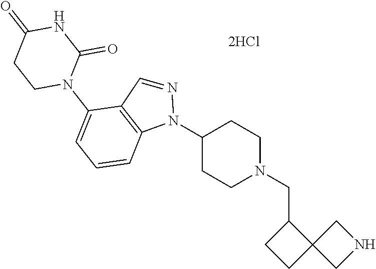 [SMILES: O=C1CCN(c2cccc3c2cnn3C2CCN(CC3CCC34CNC4)CC2)C(=O)N1 ; "2HCl"] | tert-butyl 5-formyl- 2-azaspiro [3.3]heptane- 2-carboxylate | 56.9 mg, 83% |
| | 1-(1-(1-((2-azaspiro[3.3]heptan-5- | | |
| | yl)methyl)piperidin-4-yl)-1H-indazol-4- | | |
| | yl)dihydropyrimidine-2,4(1H,3H)-dione | | |
| | dihydrochloride | | |
| | LCMS (high pH A): Rt = 0.76 min, MH⁺ = 423 | | |
| CBL32 | 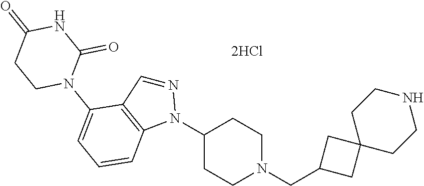 [SMILES: O=C1CCN(c2cccc3c2cnn3C2CCN(CC3CC4(CCNCC4)C3)CC2)C(=O)N1 ; "2HCl"] | tert-butyl 2-formyl- 7-azaspiro [3.5]nonane- 7-carboxylate | 8.2 mg, 11.2% |
| | 1-(1-(1-((7-azaspiro[3.5]nonan-2- | | |
| | yl)methyl)piperidin-4-yl)-1H-indazol-4- | | |
| | yl)dihydropyrimidine-2,4(1H,3H)-dione | | |
| | dihydrochloride | | |
| | LCMS (high pH A): Rt = 0.81 min, MH⁺ = 451 | | |
| CBL33 | 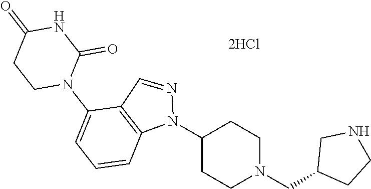 [SMILES: O=C1CCN(c2cccc3c2cnn3C2CCN(C[C@H]3CCNC3)CC2)C(=O)N1 ; "2HCl"] | tert-butyl (S)-3- formylpyrrolidine- 1-carboxylate | 43.1 mg, 66.4% |
| | (S)-1-(1-(1-(pyrrolidin-3-ylmethyl)piperidin-4-yl)-1H- | | |
| | indazol-4-yl)dihydropyrimidine-2,4(1H,3H)-dione | | |
| | dihydrochloride | | |
| | LCMS (high pH A): Rt = 0.68 min, MH⁺ = 397 | | |
| CBL34 | 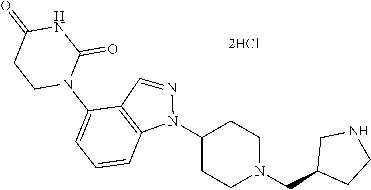 [SMILES: O=C1CCN(c2cccc3c2cnn3C2CCN(C[C@@H]3CCNC3)CC2)C(=O)N1 ; "2HCl"] | tert-butyl (R)-3- formylpyrrolidine- 1-carboxylate | 47.2 mg, 72.7% |
| | (R)-1-(1-(1-(pyrrolidin-3-ylmethyl)piperidin-4-yl)-1H- | | |
| | indazol-4-yl)dihydropyrimidine-2,4(1H,3H)-dione | | |
| | dihydrochloride | | |
| | LCMS (high pH A): Rt = 0.67 min, MH⁺ = 397 | | |
| CBL35 | 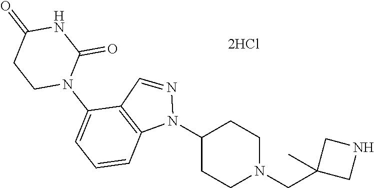 [SMILES: CC1(CN2CCC(n3ncc4c(N5CCC(=O)NC5=O)cccc43)CC2)CNC1 ; "2HCl"] | tert-butyl 3-formyl- 3-methylazetidine- 1-carboxylate | 52.9 mg, 81% |
| | 1-(1-(1-((3-methylazetidin-3-yl)methyl)piperidin-4- | | |
| | yl)-1H-indazol-4-yl)dihydropyrimidine-2,4(1H,3H)- | | |
| | dione dihydrochloride | | |
| | LCMS (high pH A): Rt = 0.66 min, MH⁺ = 397 | | |
| CBL36 | 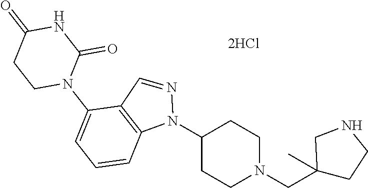 [SMILES: CC1(CN2CCC(n3ncc4c(N5CCC(=O)NC5=O)cccc43)CC2)CCNC1 ; "2HCl"] | tert-butyl 3-formyl- 3- methylpyrrolidine- 1-carboxylate | 32.3 mg, 48.2% |
| | 1-(1-(1-((3-methylpyrrolidin-3-yl)methyl)piperidin-4- | | |
| | yl)-1H-indazol-4-yl)dihydropyrimidine-2,4(1H,3H)- | | |
| | dione dihydrochloride | | |
| | LCMS (high pH A): Rt = 0.76 min, MH⁺ = 411 | | |
| CBL37 | 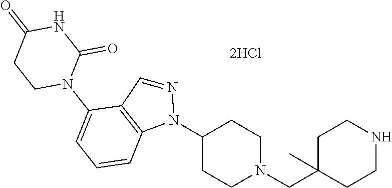 [SMILES: CC1(CN2CCC(n3ncc4c(N5CCC(=O)NC5=O)cccc43)CC2)CCNCC1 ; "2HCl"] | tert-butyl 4-formyl- 4- methylpiperidine- 1-carboxylate | 9.3 mg, 13.45% |
| | 1-(1-(1-((4-methylpiperidin-4-yl)methyl)piperidin-4- | | |
| | yl)-1H-indazol-4-yl)dihydropyrimidine-2,4(1H,3H)- | | |
| | dione dihydrochloride | | |
| | LCMS (high pH A): Rt = 0.85 min, MH⁺ = 425 | | |
| CBL38 | 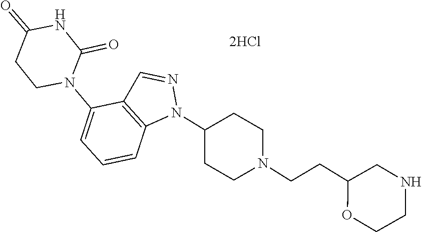 [SMILES: O=C1CCN(c2cccc3c2cnn3C2CCN(CCC3CNCCO3)CC2)C(=O)N1] | tert-butyl 2-(2- oxoethyl) morpholine- 4-carboxylate | 55.7 mg, 80% |
| | 1-(1-(1-(2-(morpholin-2-yl)ethyl)piperidin-4-yl)-1H- | | |
| | indazol-4-yl)dihydropyrimidine-2,4(1H,3H)-dione | | |
| | dihydrochloride | | |
| | LCMS (high pH A): Rt = 0.66 min, MH⁺ = 427 | | |
| CBL39 | 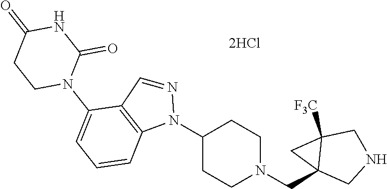 [SMILES: O=C1CCN(c2cccc3c2cnn3C2CCN(C[C@]34CNC[C@@]3(C(F)(F)F)C4)CC2)C(=O)N1 ; "2HCl"] | tert-butyl (1S,5R)- 1-formyl-5- (trifluoromethyl)- 3- azabicyclo[3.1.0] hexane-3- carboxylate | 47.1 mg, 61.2% |
| | 1-(1-(1-(((1S,5R)-5-(trifluoromethyl)-3- | | |
| | azabicyclo[3.1.0]hexan-1-yl)methyl)piperidin-4-yl)- | | |
| | 1H-indazol-4-yl)dihydropyrimidine-2,4(1H,3H)-dione | | |
| | dihydrochloride | | |
| | LCMS (high pH A): Rt = 0.88 min, MH⁺ = 477 | | |

### Androgen PROTAC Examples

The following compounds are compounds of formula (I), (Ia) or (Ib) where p and q are both 1 and the TBM is an androgen receptor binding moiety.

### Examples AR1-6

A mixture of each carboxylic acid substrate, HATU (227 mg, 0.598 mmol) and DIPEA (0.14 mL, 0.802 mmol) were dissolved in DMF (1 mL) and allowed to stir at room temperature for 30 mins. To each mixture 4-(((1r,4r)-4-aminocyclohexyl)oxy)-2-chlorobenzonitrile (may be prepared as described in Description 46; 100 mg, 0.399 mmol) was added before they were allowed to stir at room temperature in stoppered vessels for 5 h. Solvent was removed from the reactions under a stream of nitrogen before the resulting residues were diluted with ethyl acetate (2 mL) and washed with NaHCO₃ (aq) (1 mL) and brine (1 mL). The organic layer was passed through a hydrophobic frit and filtrate evaporated under a stream of nitrogen. The residues were then dissolved in DCM (1.0 mL), and TFA (0.15 mL, 1.947 mmol) was added to each vessel. The reactions were stirred at room temperature for 2 h before additional TFA (0.15 mL, 1.947 mmol) was added, and the reactions allowed to stir for 72 h. The reaction mixtures were blown down under a stream of nitrogen and the residues dried in a vacuum oven at 40° C. The resulting solids were then dissolved in MeOH (4 mL) and applied to preconditioned (with MeOH)₂ g SCX cartridges. Two column volumes of MeOH (20 mL) was passed through the cartridge before the products were eluted with 4 M NH₃ in MeOH (20 mL) and collected. The resulting samples were then dried under a stream of nitrogen. Six mixtures of 2-(4-(4-(2,4-dioxotetrahydropyrimidin-1(2H)-yl)-1H-indol-1-yl)piperidin-1-yl) acetic acid trifluoroacetate (may be prepared as described in Example ECBL4; 111 mg, 0.229 mmol), HATU (303 mg, 0.798 mmol) and DIPEA (0.21 mL, 1.202 mmol) were dissolved in DMF (1 mL) and left to stir at room temperature for 30 min. To these mixtures each of the respective substrates were added, and allowed to stir at room temperature for 16 h. Solvent was then removed from the reactions under a stream of nitrogen before the resulting residues were diluted with EtOAc (2 mL) and washed with NaHCO₃ (aq) (1 mL) and brine (1 mL). The organic layer was passed through a hydrophobic frit and filtrate evaporated under a stream of nitrogen. The crude samples were purified by the appropriate method to obtain the following compounds:

| | | | Purifi- |
|---|---|---|---|
| | | | cation |
| | | | Method; |
| | Name; | Acid | Mass; |
| Eg. | Analytical | Monomer | yield |
| AR1 | 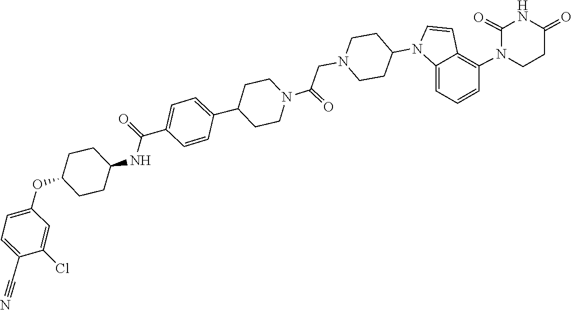 [SMILES: N#Cc1ccc(O[C@H]2CC[C@H](NC(=O)c3ccc(C4CCN(C(=O)CN5CCC(n6ccc7c(N8CCC(=O)NC8=O)cccc76)CC5)CC4)cc3)CC2)cc1Cl] | 4-(1-(tert- butoxy- carbonyl) piperidin- 4-yl) benzoic acid | MDAP (high pH) 44.7 mg, 14% |
| | N-((1r,4r)-4-(3-Chloro-4-cyanophenoxy)cyclohexyl)-4-(1- | | |
| | (2-(4-(4-(2,4-dioxotetrahydropyrimidin-1(2H)-yl)-1H- | | |
| | indol-1-yl)piperidin-1-yl)acetyl)piperidin-4-yl)benzamide | | |
| | LCMS (formic A): Rt = 0.84 min, MH⁺ = 790 | | |
| AR2 | 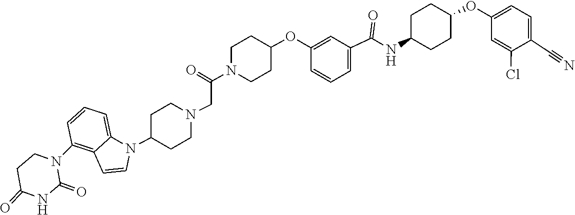 [SMILES: N#Cc1ccc(O[C@H]2CC[C@H](NC(=O)c3cccc(OC4CCN(C(=O)CN5CCC(n6ccc7c(N8CCC(=O)NC8=O)cccc76)CC5)CC4)c3)CC2)cc1Cl] | 3-((1-(tert- butoxy- carbonyl) piperidin- 4-yl)oxy) benzoic acid | 2 × MDAP (high pH) runs 22.9 mg, 7% |
| | N-((1r,4r)-4-(3-Chloro-4-cyanophenoxy)cyclohexyl)-3- | | |
| | ((1-(2-(4-(4-(2,4-dioxotetrahydropyrimidin-1(2H)-yl)-1H- | | |
| | indol-1-yl)piperidin-1-yl)acetyl)piperidin-4- | | |
| | yl)oxy)benzamide | | |
| | LCMS (formic A): Rt = 0.86 min, MH⁺ = 806 | | |
| AR3 | 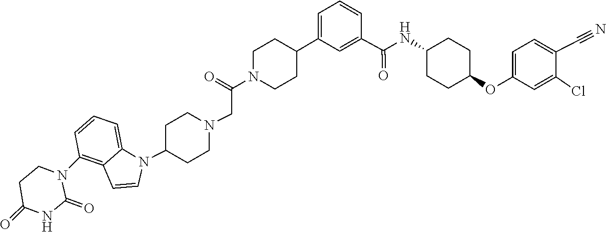 [SMILES: N#Cc1ccc(O[C@H]2CC[C@H](NC(=O)c3cccc(C4CCN(C(=O)CN5CCC(n6ccc7c(N8CCC(=O)NC8=O)cccc76)CC5)CC4)c3)CC2)cc1Cl] | 3-(1-(tert- butoxy- carbonyl) piperidin- 4-yl) benzoic acid | MDAP (high pH) then SFC 126.1 mg, 8% |
| | N-((1r,4r)-4-(3-Chloro-4-cyanophenoxy)cyclohexyl)-3-(1- | | |
| | (2-(4-(4-(2,4-dioxotetrahydropyrimidin-1(2H)-yl)-1H- | | |
| | indol-1-yl)piperidin-1-yl)acetyl)piperidin-4-yl)benzamide | | |
| | LCMS (formic A): Rt = 0.84 min, MH⁺ = 790 | | |
| AR4 | 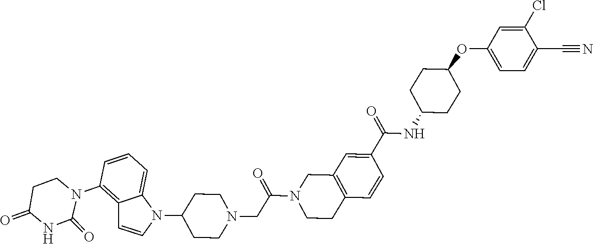 [SMILES: N#Cc1ccc(O[C@H]2CC[C@H](NC(=O)c3ccc4c(c3)CN(C(=O)CN3CCC(n5ccc6c(N7CCC(=O)NC7=O)cccc65)CC3)CC4)CC2)cc1Cl] | 2-(tert- butoxy- carbonyl)- 1,2,3,4- tetrahydro- iso- quinoline- 7- carboxylic acid | MDAP (TFA) 50.1 mg, 16% |
| | N-((1r,4r)-4-(3-Chloro-4-cyanophenoxy)cyclohexyl)-2-(2- | | |
| | (4-(4-(2,4-dioxotetrahydropyrimidin-1(2H)-yl)-1H-indol- | | |
| | 1-yl)piperidin-1-yl)acetyl)-1,2,3,4-tetrahydroisoquinoline- | | |
| | 7-carboxamide | | |
| | LCMS (formic A): Rt = 0.81 min, MH⁺ = 762 | | |
| AR5 | 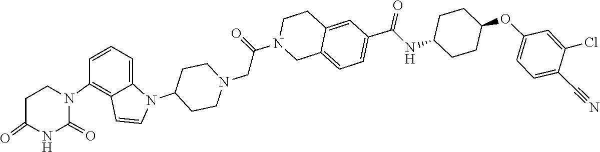 [SMILES: N#Cc1ccc(O[C@H]2CC[C@H](NC(=O)c3ccc4c(c3)CCN(C(=O)CN3CCC(n5ccc6c(N7CCC(=O)NC7=O)cccc65)CC3)C4)CC2)cc1Cl] | 2-(tert- butoxy- carbonyl)- 1,2,3,4- tetrahydro- iso- quinoline- 6- carboxylic acic | SFC2 34.7 mg, 11% |
| | N-((1r,4r)-4-(3-Chloro-4-cyanophenoxy)cyclohexyl)-2-(2- | | |
| | (4-(4-(2,4-dioxotetrahydropyrimidin-1(2H)-yl)-1H-indol- | | |
| | 1-yl)piperidin-1-yl)acetyl)-1,2,3,4-tetrahydroisoquinoline- | | |
| | 6-carboxamide | | |
| | LCMS (formic A): Rt = 0.80 min, MH⁺ = 762 | | |
| AR6 | 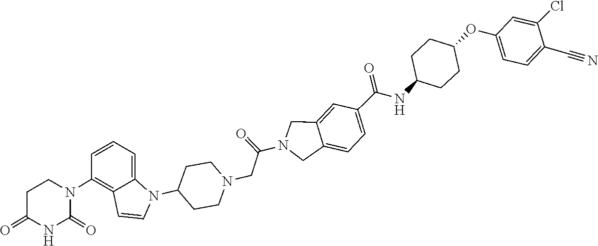 [SMILES: N#Cc1ccc(O[C@H]2CC[C@H](NC(=O)c3ccc4c(c3)CN(C(=O)CN3CCC(n5ccc6c(N7CCC(=O)NC7=O)cccc65)CC3)C4)CC2)cc1Cl] | 2-(tert- butoxy- carbonyl) iso- indoline- 5- carboxylic acid | MDAP (TFA) 29.1 mg, 10% |
| | N-((1r,4r)-4-(3-Chloro-4-cyanophenoxy)cyclohexyl)-2-(2- | | |
| | (4-(4-(2,4-dioxotetrahydropyrimidin-1(2H)-yl)-1H-indol- | | |
| | 1-yl)piperidin-1-yl)acetyl)isoindoline-5-carboxamide | | |
| | LCMS (formic A): Rt = 0.79 min, MH⁺ = 748 | | |

### Example AR7

### N-((1r,4r)-4-(3-Chloro-4-cyanophenoxy)cyclohexyl)-6-(4-(4-(2,4-dioxotetrahydropyrimidin-1(2H)-yl)-1H-indol-1-yl)-[1,4′-bipiperidin]-1′-yl)pyridazine-3-carboxamide (AR7)

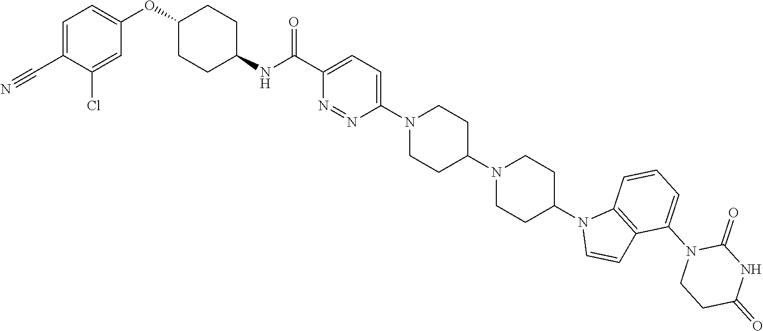 [SMILES: N#Cc1ccc(O[C@H]2CC[C@H](NC(=O)c3ccc(N4CCC(N5CCC(n6ccc7c(N8CCC(=O)NC8=O)cccc76)CC5)CC4)nn3)CC2)cc1Cl]

A mixture of 1-(1-(piperidin-4-yl)-1H-indol-4-yl)dihydropyrimidine-2,4(1H,3H)-dione (may be prepared as described in Example ECB1; 41 mg, 0.131 mmol), N-((1r,4r)-4-(3-chloro-4-cyanophenoxy)cyclohexyl)-6-(4-oxopiperidin-1-yl)pyridazine-3-carboxamide (may be prepared as described in Description 109; 60 mg, 0.132 mmol), 2-picoline borane (28 mg, 0.262 mmol) and acetic acid (7.6 μL, 0.133 mmol) were suspended in DCM (1 mL). This mixture was then allowed to stir at 50° C. for 24 h. Reaction mixture was then evaporated to dryness and dissolved in 2 mL DMSO and purified on an XBridge Prep C18 (150×30 mm, 5 μm) column eluting with 40-100% MeCN in 10 mM NH₄HCO₃ in water adjusted to pH 10 with aqueous NH₃ to give the title compound as a white solid (16 mg, 0.021 mmol). LCMS (formic A): Rt=0.81 min, MH⁺=750, 752.

### Examples AR8-AR13

### General Procedure

To a glass vial containing the appropriate aldehyde (0.085 mmol) was added a solution of 1-(1-(piperidin-4-yl)-1H-indol-4-yl)dihydropyrimidine-2,4(1H,3H)-dione (may be prepared as described in Example ECB1; 27 mg, 0.085 mmol) in anhydrous DCM (0.5 mL) and 2-picoline borane (0.027 g, 0.255 mmol) in anhydrous DCM (0.5 mL). The vial was capped and the reaction mixture stirred at room temperature for 18 h. Acetic acid (0.100 mL) was added and the solvent evaporated. The material was dissolved in DMSO (0.5 mL) and purified by MDAP (high pH). The solvent was evaporated in a vacuum centrifuge to give the following compounds:

| Ex- | Name | Alde- | Mass, |
|---|---|---|---|
| ample | Analytical | hyde | yield |
| AR8 | 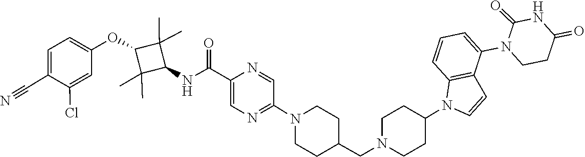 [SMILES: CC1(C)[C@H](NC(=O)c2cnc(N3CCC(CN4CCC(n5ccc6c(N7CCC(=O)NC7=O)cccc65)CC4)CC3)cn2)C(C)(C)[C@H]1Oc1ccc(C#N)c(Cl)c1] | D92 | 14.1 mg, 19% |
| | N-((1r,3r)-3-(3-Chloro-4-cyanophenoxy)-2,2,4,4- | | |
| | tetramethylcyclobutyl)-5-(4-((4-(4-(2,4- | | |
| | dioxotetrahydropyrimidin-1(2H)-yl)-1H-indol-1-yl)piperidin-1- | | |
| | yl)methyl)piperidin-1-yl)pyrazine-2-carboxamide | | |
| | LCMS (formic A): Rt = 0.94 min, MH⁺ = 792, 794 | | |
| AR9 | 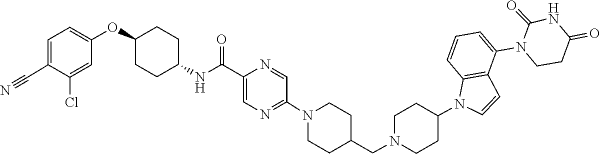 [SMILES: N#Cc1ccc(O[C@H]2CC[C@H](NC(=O)c3cnc(N4CCC(CN5CCC(n6ccc7c(N8CCC(=O)NC8=O)cccc76)CC5)CC4)cn3)CC2)cc1Cl] | D99 | 8.1 mg, 11% |
| | N-((1r,4r)-4-(3-Chloro-4-cyanophenoxy)cyclohexyl)-5-(4-((4- | | |
| | (4-(2,4-dioxotetrahydropyrimidin-1(2H)-yl)-1H-indol-1- | | |
| | yl)piperidin-1-yl)methyl)piperidin-1-yl)pyrazine-2- | | |
| | carboxamide | | |
| | LCMS (formic A): Rt = 0.84 min, MH⁺ = 764, 766 | | |
| AR10 | 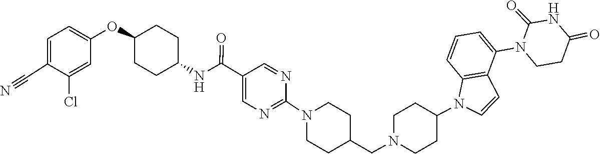 [SMILES: N#Cc1ccc(O[C@H]2CC[C@H](NC(=O)c3cnc(N4CCC(CN5CCC(n6ccc7c(N8CCC(=O)NC8=O)cccc76)CC5)CC4)nc3)CC2)cc1Cl] | D103 | 6.5 mg, 9% |
| | N-((1r,4r)-4-(3-Chloro-4-cyanophenoxy)cyclohexyl)-2-(4-((4- | | |
| | (4-(2,4-dioxotetrahydropyrimidin-1(2H)-yl)-1H-indol-1- | | |
| | yl)piperidin-1-yl)methyl)piperidin-1-yl)pyrimidine-5- | | |
| | carboxamide | | |
| | LCMS (formic A): Rt = 0.82 min, MH⁺ = 764, 766 | | |
| AR11 | 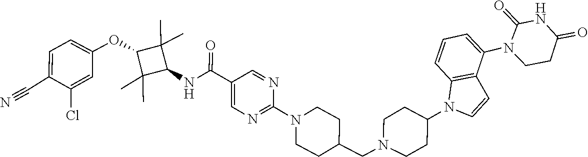 [SMILES: CC1(C)[C@H](NC(=O)c2cnc(N3CCC(CN4CCC(n5ccc6c(N7CCC(=O)NC7=O)cccc65)CC4)CC3)nc2)C(C)(C)[C@H]1Oc1ccc(C#N)c(Cl)c1] | D93 | 5.9 mg, 8% |
| | N-((1r,3r)-3-(3-Chloro-4-cyanophenoxy)-2,2,4,4- | | |
| | tetramethylcyclobutyl)-2-(4-((4-(4-(2,4- | | |
| | dioxotetrahydropyrimidin-1(2H)-yl)-1H-indol-1-yl)piperidin-1- | | |
| | yl)methyl)piperidin-1-yl)pyrimidine-5-carboxamide | | |
| | LCMS (formic A): Rt = 0.92 min, MH⁺ = 792, 794 | | |
| AR12 | 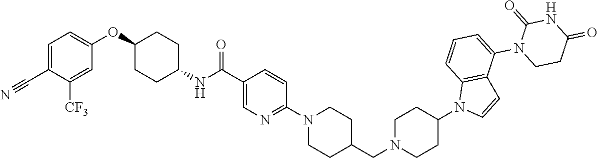 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)nc3)CC2)cc1C(F)(F)F] | D101 | 13.8 mg, 18% |
| | N-((1r,4r)-4-(4-Cyano-3- | | |
| | (trifluoromethyl)phenoxy)cyclohexyl)-6-(4-((4-(4-(2,4- | | |
| | dioxotetrahydropyrimidin-1(2H)-yl)-1H-indol-1-yl)piperidin-1- | | |
| | yl)methyl)piperidin-1-yl)nicotinamide | | |
| | LCMS (formic A): Rt = 0.80 min, MH⁺ = 797 | | |
| AR13 | 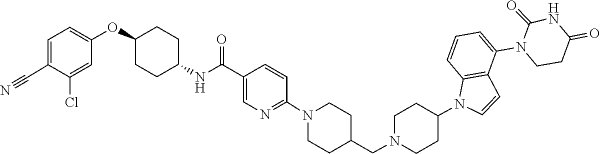 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)nc3)CC2)cc1Cl] | D142 | 8 mg, 11% |
| | N-((1r,4r)-4-(3-Chloro-4-cyanophenoxy)cyclohexyl)-6-(4-((4- | | |
| | (4-(2,4-dioxotetrahydropyrimidin-1(2H)-yl)-1H-indol-1- | | |
| | yl)piperidin-1-yl)methyl)piperidin-1-yl)nicotinamide LCMS | | |
| | (formic A): Rt = 0.76 min, MH⁺ = 763, 765 | | |

### Examples AR14-AR20

### General Procedure

An aliquot (0.4 mL) of a stock suspension of 1-(1-(piperidin-4-yl)-1H-pyrrolo[2,3-c]pyridin-4-yl)dihydropyrimidine-2,4(1H,3H)-dione (may be prepared as described in Example CB6; 0.031 g, 0.1 mmol)×7=219 mg in THF (2.8 mL)] was dispensed to the appropriate aldehyde (0.100 mmol). Sodium triacetoxyborohydride (0.106 g, 0.500 mmol) and THF (0.2 mL) was added. The mixtures were capped and stirred at room temp for 18 h. Sodium triacetoxyborohydride (0.106 g, 0.500 mmol) was added and stirred at room temperature for 18 h. The reaction was quenched with MeOH (0.5 mL) and the solvent removed to dryness. The residue was dissolved in DMSO (0.6 mL) and purified by MDAP (high pH). The solvent was evaporated using a vacuum centrifuge to give the following compounds:

| Ex- | Name | Alde- | Mass, |
|---|---|---|---|
| ample | Analytical | hyde | yield |
| AR14 | 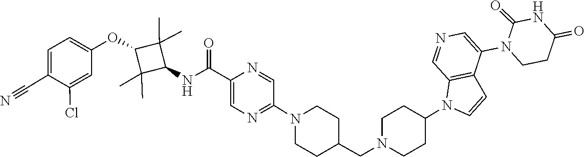 [SMILES: CC1(C)[C@H](NC(=O)c2cnc(N3CCC(CN4CCC(n5ccc6c(N7CCC(=O)NC7=O)cncc65)CC4)CC3)cn2)C(C)(C)[C@H]1Oc1ccc(C#N)c(Cl)c1] | D92 | 3.5 mg, 5% |
| | N-((1r,3r)-3-(3-Chloro-4-cyanophenoxy)-2,2,4,4- | | |
| | tetramethylcyclobutyl)-5-(4-((4-(4-(2,4- | | |
| | dioxotetrahydropyrimidin-1(2H)-yl)-1H-pyrrolo[2,3-c]pyridin-1- | | |
| | yl)piperidin-1-yl)methyl)piperidin-1-yl)pyrazine-2-carboxamide | | |
| | LCMS (formic A): Rt = 0.75 min, MH⁺ = 793, 795 | | |
| AR15 | 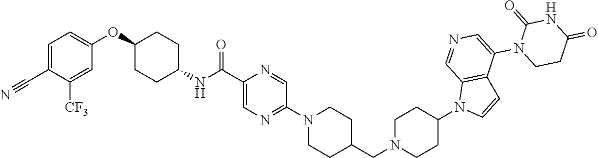 [SMILES: N#Cc1ccc(O[C@H]2CC[C@H](NC(=O)c3cnc(N4CCC(CN5CCC(n6ccc7c(N8CCC(=O)NC8=O)cncc76)CC5)CC4)cn3)CC2)cc1C(F)(F)F] | D98 | 9.8 mg, 11% |
| | N-((1r,4r)-4-(4-Cyano-3-(trifluoromethyl)phenoxy)cyclohexyl)- | | |
| | 5-(4-((4-(4-(2,4-dioxotetrahydropyrimidin-1(2H)-yl)-1H- | | |
| | pyrrolo[2,3-c]pyridin-1-yl)piperidin-1-yl)methyl)piperidin-1- | | |
| | yl)pyrazine-2-carboxamide LCMS (formic A): Rt = 0.69 min, | | |
| | MH⁺ = 798 | | |
| AR16 | 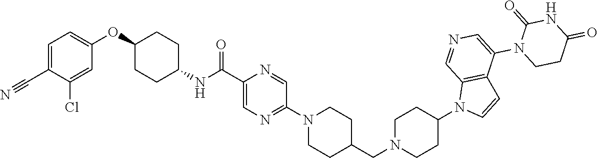 [SMILES: N#Cc1ccc(O[C@H]2CC[C@H](NC(=O)c3cnc(N4CCC(CN5CCC(n6ccc7c(N8CCC(=O)NC8=O)cncc76)CC5)CC4)cn3)CC2)cc1Cl] | D99 | 7.7 mg, 9% |
| | N-((1r,4r)-4-(3-Chloro-4-cyanophenoxy)cyclohexyl)-5-(4-((4- | | |
| | (4-(2,4-dioxotetrahydropyrimidin-1(2H)-yl)-1H-pyrrolo[2,3- | | |
| | c]pyridin-1-yl)piperidin-1-yl)methyl)piperidin-1-yl)pyrazine-2- | | |
| | carboxamide LCMS (formic A): Rt = 0.65 min, MH⁺ = 766 | | |
| AR17 | 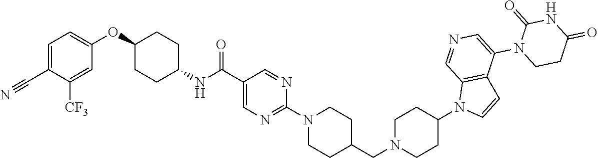 [SMILES: N#Cc1ccc(O[C@H]2CC[C@H](NC(=O)c3cnc(N4CCC(CN5CCC(n6ccc7c(N8CCC(=O)NC8=O)cncc76)CC5)CC4)nc3)CC2)cc1C(F)(F)F] | D100 | 21.9 mg, 25% |
| | N-((1r,4r)-4-(4-Cyano-3-(trifluoromethyl)phenoxy)cyclohexyl)- | | |
| | 2-(4-((4-(4-(2,4-dioxotetrahydropyrimidin-1(2H)-yl)-1H- | | |
| | pyrrolo[2,3-c]pyridin-1-yl)piperidin-1-yl)methyl)piperidin-1- | | |
| | yl)pyrimidine-5-carboxamide | | |
| | LCMS (formic A): Rt = 0.67 min, MH⁺ = 799 | | |
| AR18 | 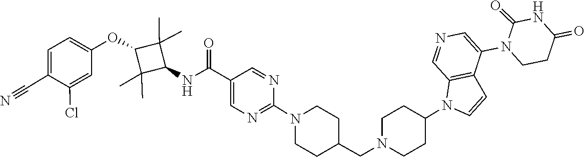 [SMILES: CC1(C)[C@H](NC(=O)c2cnc(N3CCC(CN4CCC(n5ccc6c(N7CCC(=O)NC7=O)cncc65)CC4)CC3)nc2)C(C)(C)[C@H]1Oc1ccc(C#N)c(Cl)c1] | D93 | 18.5 mg, 21%. |
| | N-((1r,3r)-3-(3-Chloro-4-cyanophenoxy)-2,2,4,4- | | |
| | tetramethylcyclobutyl)-2-(4-((4-(4-(2,4- | | |
| | dioxotetrahydropyrimidin-1(2H)-yl)-1H-pyrrolo[2,3-c]pyridin-1- | | |
| | yl)piperidin-1-yl)methyl)piperidin-1-yl)pyrimidine-5- | | |
| | carboxamide | | |
| | LCMS (formic A): Rt = 0.74 min, MH⁺ = 793, 795 | | |
| AR19 | 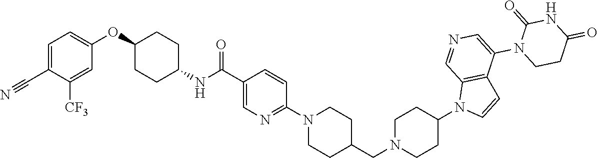 [SMILES: N#Cc1ccc(O[C@H]2CC[C@H](NC(=O)c3ccc(N4CCC(CN5CCC(n6ccc7c(N8CCC(=O)NC8=O)cncc76)CC5)CC4)nc3)CC2)cc1C(F)(F)F] | D101 | 25.4 mg, 29% |
| | N-((1r,4r)-4-(4-Cyano-3-(trifluoromethyl)phenoxy)cyclohexyl)- | | |
| | 6-(4-((4-(4-(2,4-dioxotetrahydropyrimidin-1(2H)-yl)-1H- | | |
| | pyrrolo[2,3-c]pyridin-1-yl)piperidin-1-yl)methyl)piperidin-1- | | |
| | yl)nicotinamide | | |
| | LCMS (formic A): Rt = 0.62 min, MH⁺ = 798 | | |
| AR20 | 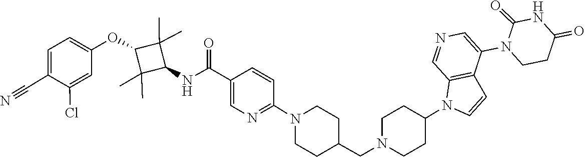 [SMILES: CC1(C)[C@H](NC(=O)c2ccc(N3CCC(CN4CCC(n5ccc6c(N7CCC(=O)NC7=O)cncc65)CC4)CC3)nc2)C(C)(C)[C@H]1Oc1ccc(C#N)c(Cl)c1] | D96 | 21.2 mg, 24% |
| | N-((1r,3r)-3-(3-Chloro-4-cyanophenoxy)-2,2,4,4- | | |
| | tetramethylcyclobutyl)-6-(4-((4-(4-(2,4- | | |
| | dioxotetrahydropyrimidin-1(2H)-yl)-1H-pyrrolo[2,3-c]pyridin-1- | | |
| | yl)piperidin-1-yl)methyl)piperidin-1-yl)nicotinamide | | |
| | LCMS (formic A): Rt = 0.69 min, MH⁺ = 792 | | |

### Example AR21

### N-((1r,4r)-4-(4-Cyano-3-(trifluoromethyl)phenoxy)cyclohexyl)-5-(4-((4-(4-(2,4-dioxotetrahydropyrimidin-1(2H)-yl)-1H-indol-1-yl)piperidin-1-yl)methyl)piperidin-1-yl)pyrazine-2-carboxamide formate (EAR21)

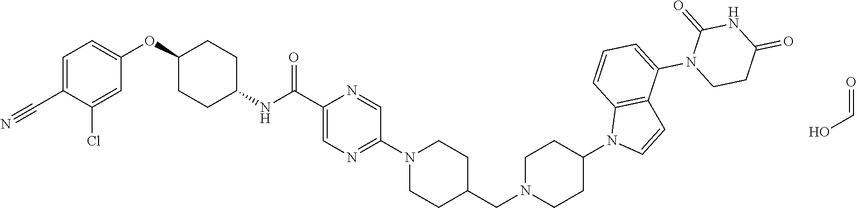 [SMILES: N#Cc1ccc(O[C@H]2CC[C@H](NC(=O)c3cnc(N4CCC(CN5CCC(n6ccc7c(N8CCC(=O)NC8=O)cccc76)CC5)CC4)cn3)CC2)cc1Cl ; O=CO]

A mixture of 1-(1-(piperidin-4-yl)-1H-indol-4-yl)dihydropyrimidine-2,4(1H,3H)-dione (may be prepared as described in Example CB1; 50 mg, 0.160 mmol), N-((1r,4r)-4-(4-cyano-3-(trifluoromethyl)phenoxy)cyclohexyl)-5-(4-formylpiperidin-1-yl)pyrazine-2-carboxamide (may be prepared as described in Description 98; 80 mg, 0.160 mmol) and sodium triacetoxyborohydride (100 mg, 0.472 mmol) was suspended in DCM (2 mL) and then left to stir at room temperature for 16 h in a sealed vessel. The reaction was then washed with aq saturated NaHCO₃ (4 mL) and brine (4 mL), and the organic layer then passed through a hydrophobic frit, and the filtrate evaporated to dryness using rotary evaporation. Crude sample was purified by MDAP (formic). The appropriate fractions were combined and the solvent evaporated under a N₂ stream to give the title compound as an off white solid (49 mg, 0.058 mmol). LCMS (formic A): Rt=0.83 min, MH⁺=798.

### Example AR22

### N-((1r,4r)-4-(4-Cyano-3-(trifluoromethyl)phenoxy)cyclohexyl)-5-(4-((4-(4-(2,4-dioxotetrahydropyrimidin-1(2H)-yl)-1H-indol-1-yl)piperidin-1-yl)methyl)piperidin-1-yl)pyrazine-2-carboxamide hydrochloride (EAR22)

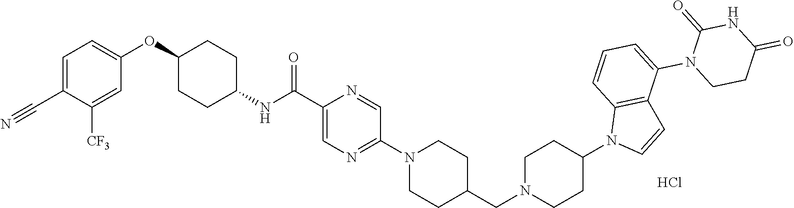 [SMILES: N#Cc1ccc(O[C@H]2CC[C@H](NC(=O)c3cnc(N4CCC(CN5CCC(n6ccc7c(N8CCC(=O)NC8=O)cccc76)CC5)CC4)cn3)CC2)cc1C(F)(F)F ; Cl]

A solution of N-((1r,4r)-4-(4-cyano-3-(trifluoromethyl)phenoxy)cyclohexyl)-5-(4-((4-(4-(2,4-dioxotetrahydropyrimidin-1(2H)-yl)-1H-indol-1-yl)piperidin-1-yl)methyl)piperidin-1-yl)pyrazine-2-carboxamide formate (may be prepared as described in Example AR21; 42 mg, 0.050 mmol) was dissolved in DCM (1.0 mL) and washed with sat. NaHCO₃ (aq) (2×1.0 mL). The organic layer was passed through a hydrophobic frit and the filtrate evaporated under a stream of nitrogen. The solid was dried in a high vacuum oven to give the assumed free base (36 mg, 0.045 mmol). This was dissolved in 1,4-dioxane (0.50 mL), treated with 4 M HCl in dioxane (17 μL, 0.067 mmol) and stirred at room temperature in a stoppered vessel for 2 h. The mixture was evaporated under a stream of nitrogen and the solid dried in a vacuum oven for 16 h to give the title compound as an off white solid (34 mg, 0.041 mmol). LCMS (high pH A): Rt=1.33 min, MH⁺=798.

### Example AR23

### N-((1r,4r)-4-(3-Chloro-4-cyanophenoxy)cyclohexyl)-6-(4-((4-(4-(2,4-dioxotetrahydropyrimidin-1(2H)-yl)-1H-indol-1-yl)piperidin-1-yl)methyl)piperidin-1-yl)pyridazine-3-carboxamide (EAR23)

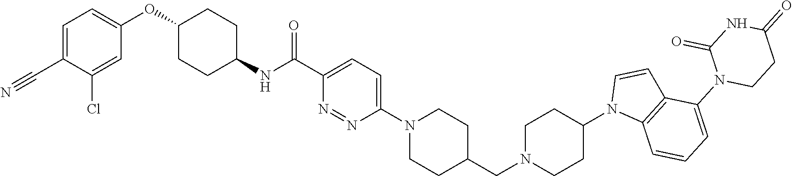 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)nn3)CC2)cc1Cl]

To a stirred solution of N-((1r,4r)-4-(3-chloro-4-cyanophenoxy)cyclohexyl)-6-(4-formylpiperidin-1-yl)pyridazine-3-carboxamide (may be prepared as described in Description 95; 65 mg, 0.139 mmol) and 1-(1-(piperidin-4-yl)-1H-indol-4-yl)dihydropyrimidine-2,4(1H,3H)-dione (may be prepared as described in Example CB1; 43.4 mg, 0.139 mmol) in DCM (1500 μl) and (DMF) (500 μl) was added sodium triacetoxyborohydride (88 mg, 0.417 mmol), and the reaction mixture was stirred at room temperature for 30 min. The reaction mixture was diluted with saturated aqueous sodium hydrogen carbonate (10 mL) and extracted with DCM (3×10 mL). The organic layers were combined, passed through a hydrophobic frit and the solvent removed in vacuo. The residue was purified by MDAP (high pH) to give the title compound (50 mg, 0.065 mmol, 47% yield) as a white solid. LCMS (high pH A): Rt=1.27 min, MH⁺=764, 766. ¹H NMR (400 MHZ, DMSO-d₆) δ ppm 10.31 (s, 1H), 8.56 (d, J=7.9 Hz, 1H), 7.86 (d, J=8.4 Hz, 1H), 7.81 (d, J=9.4 Hz, 1H), 7.54 (d, J=3.0 Hz, 1H), 7.51 (d, J=8.4 Hz, 1H), 7.39 (d, J=2.5 Hz, 1H), 7.34 (d, J=9.8 Hz, 1H), 7.12-7.18 (m, 2H), 6.97 (d, J=7.4 Hz, 1H), 6.42 (d, J=3.0 Hz, 1H), 4.44-4.60 (m, 3H), 4.31-4.44 (m, 1H), 3.83-3.93 (m, 1H), 3.79 (t, J=6.6 Hz, 2H), 2.96-3.11 (m, 4H), 2.77 (t, J=6.6 Hz, 2H), 2.25 (br d, J=6.9 Hz, 2H), 2.07-2.22 (m, 4H), 1.82-2.07 (m, 9H), 1.59-1.73 (m, 2H), 1.44-1.59 (m, 2H), 1.08-1.22 (m, 2H).

### Example AR24

### N-((1r,4r)-4-(4-Cyano-3-(trifluoromethyl)phenoxy)cyclohexyl)-6-(4-((4-(4-(2,4-dioxotetrahydropyrimidin-1(2H)-yl)-1H-indol-1-yl)piperidin-1-yl)methyl)piperidin-1-yl)pyridazine-3-carboxamide (EAR24)

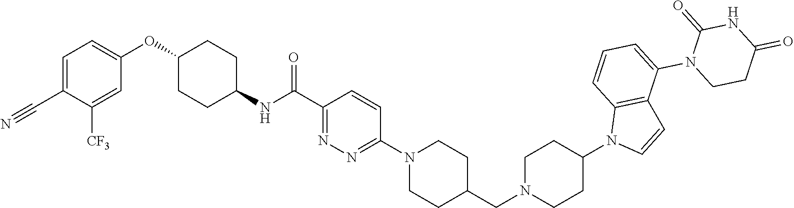 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)nn3)CC2)cc1C(F)(F)F]

A microwave vial was charged with N-((1r,4r)-4-(4-cyano-3-(trifluoromethyl)phenoxy)cyclohexyl)-6-(4-formylpiperidin-1-yl)pyridazine-3-carboxamide (may be prepared as described in Description 97; 100 mg, 0.120 mmol), 1-(1-(piperidin-4-yl)-1H-indol-4-yl)dihydropyrimidine-2,4(1H,3H)-dione (may be prepared as described in Example CB1; 37.7 mg, 0.121 mmol) and DCM (1424 μL). To the stirred mixture was added sodium triacetoxyborohydride (76 mg, 0.359 mmol), and the mixture was stirred at room temperature for 30 min, then left to stand at room temperature for 1 h. The reaction mixture was quenched with saturated aqueous sodium bicarbonate solution (20 mL) and extracted with DCM (3×25 mL). The combined organic extracts were dried by passing through a hydrophobic frit and the solvent removed in vacuo to give a yellow solid which was dissolved in 1:1 DMSO:MeOH (1 mL) and purified by MDAP (high pH). Appropriate fractions were collected and the solvent removed under a stream of nitrogen to give the title compound (50.7 mg, 0.064 mmol, 53% yield) as a light brown solid. LCMS (high pH A): Rt=1.29 min, MH⁺=798.

### Example AR25

### N-((1r,4r)-4-(4-Cyano-3-(trifluoromethyl)phenoxy)cyclohexyl)-6-(4-((4-(4-(2,4-dioxotetrahydropyrimidin-1(2H)-yl)-1H-indol-1-yl)piperidin-1-yl)methyl)piperidin-1-yl)pyridazine-3-carboxamide hydrochloride (EAR25)

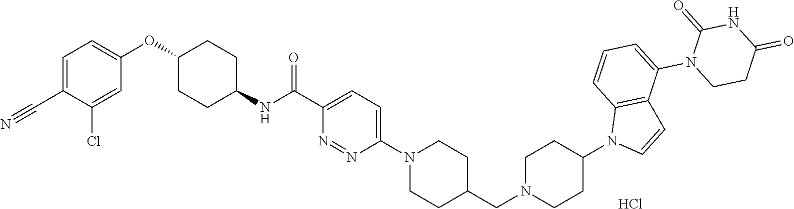 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)nn3)CC2)cc1Cl ; Cl]

To N-((1r,4r)-4-(4-cyano-3-(trifluoromethyl)phenoxy)cyclohexyl)-6-(4-((4-(4-(2,4-dioxotetrahydropyrimidin-1(2H)-yl)-1H-indol-1-yl)piperidin-1-yl)methyl)piperidin-1-yl)pyridazine-3-carboxamide (may be prepared as described in Example EAR24; 36.88 mg, 0.047 mmol) suspended in 1,4-Dioxane (200 μL) and stirred at room temperature was added HCl (4 M in Dioxane) (45.4 μL, 0.182 mmol). The reaction mixture was stirred at room temperature for 4 h then dried under a stream of nitrogen for 4 h, then further dried in a vacuum oven overnight to give the title compound (35.6 mg, 0.044 mmol, 90% yield) as a pale yellow solid. LCMS (high pH A): Rt=1.30 min, MH⁺=798.

### Example AR26

### N-((1r,4r)-4-(3-Chloro-4-cyanophenoxy)cyclohexyl)-6-(4-((4-((4-(2,4-dioxotetrahydropyrimidin-1(2H)-yl)-1H-indol-1-yl)methyl)piperidin-1-yl)methyl)piperidin-1-yl)pyridazine-3-carboxamide hydrochloride (EAR26)

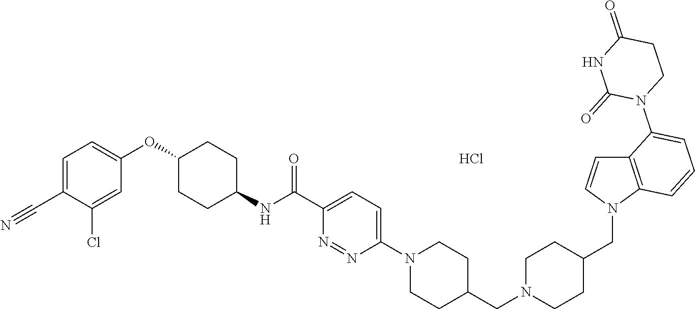 [SMILES: N#Cc1ccc(O[C@H]2CC[C@H](NC(=O)c3ccc(N4CCC(CN5CCC(Cn6ccc7c(N8CCC(=O)NC8=O)cccc76)CC5)CC4)nn3)CC2)cc1Cl ; Cl]

A microwave vial was charged with N-((1r,4r)-4-(3-chloro-4-cyanophenoxy)cyclohexyl)-6-(4-formylpiperidin-1-yl)pyridazine-3-carboxamide (may be prepared as described in Description 95; 85 mg, 0.182 mmol), DCM (0.7 mL) and a solution of 1-(1-(piperidin-4-ylmethyl)-1H-indol-4-yl)dihydropyrimidine-2,4(1H,3H)-dione (may be prepared as described in Example ECB2; 59.9 mg, 0.183 mmol) in DCM (1.4 mL). To the stirred mixture was added sodium triacetoxyborohydride (115 mg, 0.545 mmol), and the mixture was stirred at room temperature for 2.5 h. The reaction mixture was quenched with saturated aqueous sodium bicarbonate solution (20 mL) and extracted with DCM (3×25 mL). The combined organic extracts were dried by passing through a hydrophobic frit, and the solvent was removed in vacuo. To this material was added 1:1 DMSO:MeOH (2 mL), and the mixture was purified by MDAP (high pH) in 2 portions. The appropriate fractions were collected and the solvent removed under a stream of nitrogen to give the product as an off-white solid. To a solution of this material in 1,4-dioxane (100 μL) stirred at room temperature was added HCl (4 M in dioxane) (15 μL, 0.058 mmol). The resulting suspension was stirred at room temperature for 2 h. The solvent was removed under a stream of nitrogen then further dried in a vacuum oven to give the title compound (17.8 mg, 0.022 mmol, 12% yield) as a yellow solid. LCMS (high pH A): Rt=1.28 min, MH⁺=778, 780.

### Example AR27

### N-((1r,4r)-4-(3-Chloro-4-cyanophenoxy)cyclohexyl)-6-(4-((4-(2,4-dioxotetrahydropyrimidin-1(2H)-yl)-1H-indol-1-yl)methyl)-[1,4′-bipiperidin]-1′-yl)pyridazine-3-carboxamide hydrochloride (EAR27)

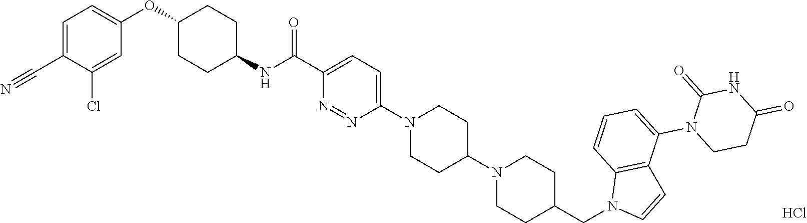 [SMILES: N#Cc1ccc(O[C@H]2CC[C@H](NC(=O)c3ccc(N4CCC(N5CCC(Cn6ccc7c(N8CCC(=O)NC8=O)cccc76)CC5)CC4)nn3)CC2)cc1Cl ; Cl]

To N-((1r,4r)-4-(3-Chloro-4-cyanophenoxy)cyclohexyl)-6-(4-oxopiperidin-1-yl)pyridazine-3-carboxamide (may be prepared as described in Description 109; 140 mg, 0.308 mmol) in a microwave vial was added a solution of 1-(1-(piperidin-4-ylmethyl)-1H-indol-4-yl)dihydropyrimidine-2,4(1H,3H)-dione (may be prepared as described in Example ECB2; 100 mg, 0.306 mmol) in DCM, followed by 2-picoline borane (65.3 mg, 0.611 mmol) and acetic acid (0.018 mL, 0.310 mmol). The vial was sealed and the mixture was stirred at 50° C. for 21.5 h. The solvent was removed under a stream of nitrogen, then dissolved in a minimum quantity of DMSO:MeOH 1:1 and purified by MDAP (high pH). Appropriate fractions were collected and the solvent removed in vacuo. The resulting solid was dissolved in minimal DCM and transferred to a weighed vial and the solvent removed under a stream of nitrogen. To this material in 1,4-dioxane (300 μL) stirred at room temperature was added HCl (4 M in Dioxane) (53.8 μL, 0.215 mmol). The resulting suspension was stirred at room temperature for 2 h. The solvent was removed under a stream of nitrogen and further dried in a vacuum oven to give the title compound (57.2 mg, 0.071 mmol, 23% yield) as a yellow solid. LCMS (high pH A): Rt=1.20 min, MH⁺=764, 766.

### Example AR28

### N-((1r,4r)-4-(3-Chloro-4-cyanophenoxy)cyclohexyl)-4-(4-(4-(2,4-dioxotetrahydropyrimidin-1(2H)-yl)-1H-indol-1-yl)-[1,3′-bipiperidin]-1′-yl)benzamide (EAR28)

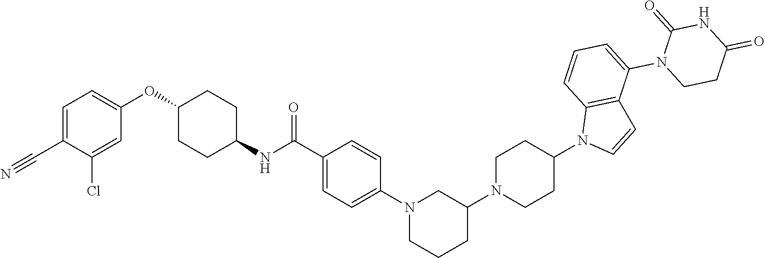 [SMILES: N#Cc1ccc(O[C@H]2CC[C@H](NC(=O)c3ccc(N4CCCC(N5CCC(n6ccc7c(N8CCC(=O)NC8=O)cccc76)CC5)C4)cc3)CC2)cc1Cl]

A mixture of 1-(4-(((1r,4r)-4-(3-chloro-4-cyanophenoxy)cyclohexyl)carbamoyl)phenyl)piperidin-3-yl 4-methylbenzenesulfonate (may be prepared as described in Description 116; 36 mg, 0.059 mmol), 1-(1-(piperidin-4-yl)-1H-indol-4-yl)dihydropyrimidine-2,4(1H,3H)-dione (may be prepared as described in Example CB1; 23.5 mg, 0.075 mmol) and DIPEA (0.021 mL, 0.120 mmol) was stirred in DMSO (3 mL) at room temperature for 17 h. The reaction mixture was heated at 60° C. for 2 h. The reaction mixture was partitioned between EtOAc (15 mL) and water (15 mL). The aqueous phase was separated. The organic phase was washed with water (10 mL) and brine (10 mL). The organic phase was dried and evaporated. The residue was purified by MDAP (formic) to give the title compound (6.4 mg, 8.55 μmol, 14% yield), as a white solid. LCMS (formic A): Rt=1.23 min, MH⁺=747,750.

### Example AR29

### N-((1r,3r)-3-(3-chloro-4-cyanophenoxy)-2,2,4,4-tetramethylcyclobutyl)-6-(4-((4-(4-(2,4-dioxotetrahydropyrimidin-1(2H)-yl)-1H-indol-1-yl)piperidin-1-yl)methyl)piperidin-1-yl)nicotinamide formate (EAR29)

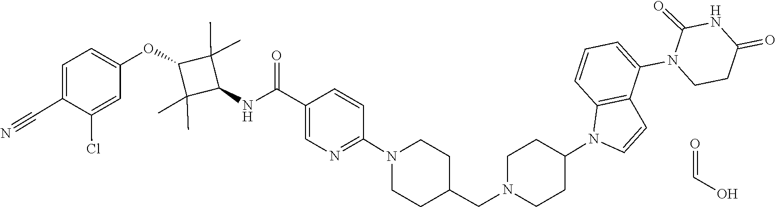 [SMILES: CC1(C)[C@H](NC(=O)c2ccc(N3CCC(CN4CCC(n5ccc6c(N7CCC(=O)NC7=O)cccc65)CC4)CC3)nc2)C(C)(C)[C@H]1Oc1ccc(C#N)c(Cl)c1 ; O=CO]

A mixture of 1-(1-(piperidin-4-yl)-1H-indol-4-yl)dihydropyrimidine-2,4(1H,3H)-dione (may be prepared as described in Example CB1; 50 mg, 0.160 mmol), N-((1r,3r)-3-(3-chloro-4-cyanophenoxy)-2,2,4,4-tetramethylcyclobutyl)-6-(4-formylpiperidin-1-yl)nicotinamide (may be prepared as described in Description 96; 80 mg, 0.162 mmol) and sodium triacetoxyborohydride (100 mg, 0.472 mmol) was suspended in DCM (2 mL) and then left to stir at room temperature for 16 h in a sealed vessel. Reaction was then washed with aq saturated NaHCO₃ (4 mL) and brine (4 mL), the organic layer then passed through a hydrophobic frit and the filtrate evaporated to dryness using rotary evaporation. Crude sample was purified by MDAP (formic). The appropriate fractions were combined and the solvent evaporated under a N₂ stream to give the title compound as an off white solid (45.6 mg, 0.054 mmol, 34%). LCMS (formic A): Rt=0.85 min, MH⁺=791, 793.

### Example AR30

### N-((1r,4r)-4-(3-Chloro-4-cyanophenoxy)cyclohexyl)-6-(4-((4-(4-(2,4-dioxotetrahydropyrimidin-1(2H)-yl)-1H-indol-1-yl)-3,3-difluoropiperidin-1-yl)methyl)piperidin-1-yl)pyridazine-3-carboxamide (EAR30)

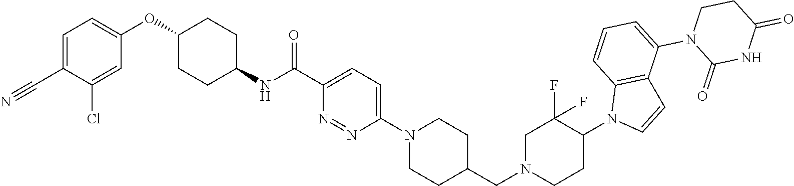 [SMILES: N#Cc1ccc(O[C@H]2CC[C@H](NC(=O)c3ccc(N4CCC(CN5CCC(n6ccc7c(N8CCC(=O)NC8=O)cccc76)C(F)(F)C5)CC4)nn3)CC2)cc1Cl]

N-((1r,4r)-4-(3-chloro-4-cyanophenoxy)cyclohexyl)-6-(4-formylpiperidin-1-yl)pyridazine-3-carboxamide (may be prepared as described in Description 95; 81 mg, 0.172 mmol) and 1-(1-(3,3-difluoropiperidin-4-yl)-1H-indol-4-yl)dihydropyrimidine-2,4(1H,3H)-dione (may be prepared as described in Example CB3; 50 mg, 0.144 mmol) in DCM (2 mL) were stirred at room temperature for 10 min. Sodium triacetoxyborohydride (91 mg, 0.431 mmol) was added, and the reaction was stirred at room temperature overnight. The reaction mixture was diluted with DCM, NaHCO₃ and brine were added and shaken vigorously. The organic layer was passed through a hydrophobic frit and blown down. The residue was purified by MDAP (high pH) in two batches and product-containing fractions were concentrated, and the residue was dried in a vac oven at 40° C. overnight to give the title compound (62 mg, 0.074 mmol, 51% yield) as a white powder. LCMS (high pH A): Rt=1.28 min, MH⁺=800, 802.

### Example AR31

### N-((1r,4r)-4-(3-Chloro-4-cyanophenoxy)cyclohexyl)-6-(4-((4-(4-(2,4-dioxotetrahydropyrimidin-1(2H)-yl)-6-fluoro-1H-indol-1-yl)piperidin-1-yl)methyl)piperidin-1-yl)pyridazine-3-carboxamide hydrochloride (EAR31)

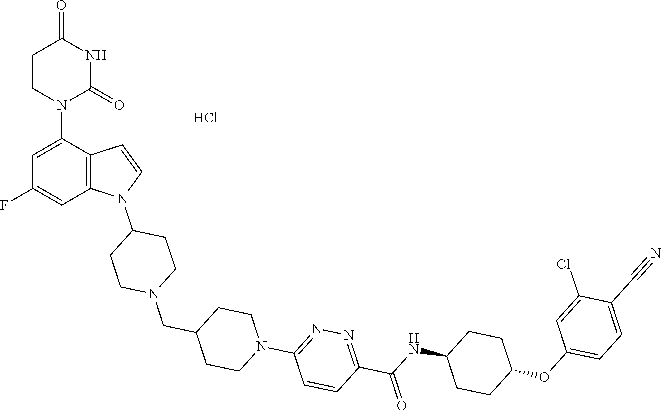 [SMILES: N#Cc1ccc(O[C@H]2CC[C@H](NC(=O)c3ccc(N4CCC(CN5CCC(n6ccc7c(N8CCC(=O)NC8=O)cc(F)cc76)CC5)CC4)nn3)CC2)cc1Cl ; Cl]

A mixture of 1-(6-fluoro-1-(piperidin-4-yl)-1H-indol-4-yl)-3-((2-(trimethylsilyl)ethoxy)methyl)dihydropyrimidine-2,4(1H,3H)-dione (may be prepared as described in Description 11; 112 mg, 0.243 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 95 mg, 0.203 mmol) in DCM (2.0 mL) was stirred in a stoppered vessel at room temperature for 10 min. Sodium triacetoxyborohydride (129 mg, 0.609 mmol) was added and the reaction mixture stirred in a stoppered vessel at room temperature for 3 h. The reaction mixture was washed sequentially with saturated NaHCO₃ (aq) (2.0 mL) and brine (2.0 mL), and the organic layer passed through a hydrophobic frit. The filtrate was evaporated in vacuo and the residue loaded in DCM (2 mL) and purified on a 12 g silica cartridge using a gradient of 0-100% EtOAc:ethanol (3:1) in MTBE over 12 column volumes. The appropriate fractions were combined and the solvent evaporated in vacuo to give N-((1r,4r)-4-(3-chloro-4-cyanophenoxy)cyclohexyl)-6-(4-((4-(4-(2,4-dioxo-3-((2-(trimethylsilyl)ethoxy)methyl)tetrahydropyrimidin-1(2H)-yl)-6-fluoro-1H-indol-1-yl)piperidin-1-yl)methyl)piperidin-1-yl)pyridazine-3-carboxamideas an off white solid (154 mg, 0.169 mmol). A solution of N-((1r,4r)-4-(3-chloro-4-cyanophenoxy)cyclohexyl)-6-(4-((4-(4-(2,4-dioxo-3-((2-(trimethylsilyl)ethoxy)methyl)tetrahydropyrimidin-1(2H)-yl)-6-fluoro-1H-indol-1-yl)piperidin-1-yl)methyl)piperidin-1-yl)pyridazine-3-carboxamide (154 mg, 0.169 mmol) in DCM (2.0 mL) was treated with TFA (0.390 mL, 5.06 mmol) and the solution left to stand in a stoppered vessel at room temperature for 1.5 h. The mixture was evaporated under a stream of nitrogen and the residue dissolved in MeOH (1.0 mL) and DCM (2.0 mL). The solution was treated with 4 M ammonia in MeOH (4.0 mL, 16.00 mmol) and allowed to stir in a stoppered vessel at room temperature for 1 h. The mixture was evaporated in vacuo and the residue suspended in DCM (5 mL). The mixture was washed sequentially with saturated NaHCO₃ (aq) (5 mL) and brine (5 mL). The organic layer was passed through a hydrophobic frit and the filtrate evaporated onto Florisil. The Florisil-adsorbed material was purified on a 12 g silica cartridge using a gradient of 0-7.5% MeOH in DCM (with 1% triethylamine) over 14 column volumes. The appropriate fractions were combined and the solvent evaporated in vacuo. The material was dried in a vacuum oven to give the free base (approximately 87 mg). This was dissolved in 1,4-dioxane (1.0 mL) and treated with 4 M HCl in 1,4-dioxane (0.042 mL, 0.167 mmol). The mixture was stirred in a stoppered vessel at room temperature for 2 h then evaporated under a stream of nitrogen. The solid was dried in a vacuum oven to give the title compound as a beige solid (100 mg, 0.122 mmol). LCMS (high pH A): Rt=1.30 min; MH⁺=782, 783, 784.

### Example AR32

### 6-(4-((4-(6-Chloro-4-(2,4-dioxotetrahydropyrimidin-1(2H)-yl)-1H-indol-1-yl)piperidin-1-yl)methyl)piperidin-1-yl)-N-((1r,4r)-4-(3-chloro-4-cyanophenoxy)cyclohexyl)pyridazine-3-carboxamide hydrochloride (EAR32)

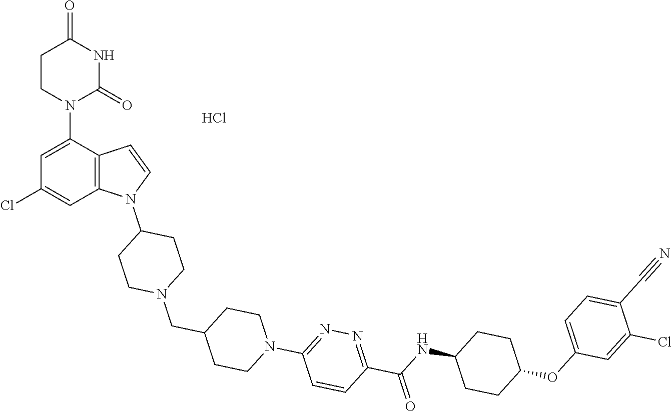 [SMILES: N#Cc1ccc(O[C@H]2CC[C@H](NC(=O)c3ccc(N4CCC(CN5CCC(n6ccc7c(N8CCC(=O)NC8=O)cc(Cl)cc76)CC5)CC4)nn3)CC2)cc1Cl ; Cl]

To a mixture of 1-(6-chloro-1-(piperidin-4-yl)-1H-indol-4-yl)dihydropyrimidine-2,4(1H,3H)-dione (12 mg, 0.035 mmol) (may be prepared as described in Example CB9) and N-((1r,4r)-4-(3-chloro-4-cyanophenoxy)cyclohexyl)-6-(4-formylpiperidin-1-yl)pyridazine-3-carboxamide (may be prepared as described in Description 95; 20 mg, 0.043 mmol) in anhydrous DCM (1.0 mL) was added sodium triacetoxyborohydride (22.00 mg, 0.104 mmol) and the reaction mixture stirred in a stoppered vessel at room temperature for 15 h. The reaction mixture was washed sequentially with saturated NaHCO₃ (aq (2 mL) and brine (2 mL). The residue was purified by by MDAP (high pH). The appropriate fractions were combined and the solvent evaporated in vacuo to give 6-(4-((4-(6-chloro-4-(2,4-dioxotetrahydropyrimidin-1(2H)-yl)-1H-indol-1-yl)piperidin-1-yl)methyl)piperidin-1-yl)-N-((1r,4r)-4-(3-chloro-4-cyanophenoxy)cyclohexyl)pyridazine-3-carboxamide (16 mg, 0.020 mmol). The solid was dissolved in 1,4-dioxane (0.4 mL) and treated with 4 M HCl in 1,4-dioxane (7.50 μl, 0.030 mmol). The mixture was stirred at room temperature for 2 h. The mixture was evaporated under a stream of nitrogen and the solid dried in a vacuum oven for 18 h to give the title compound as an off white solid (17 mg, 0.020 mmol). LCMS (high pH A): Rt=1.35 min, MH⁺=798, 799, 800, 801.

### Example AR33

### N-((1r,4r)-4-(3-Chloro-4-cyanophenoxy)cyclohexyl)-6-(4-((4-(4-(2,4-dioxotetrahydropyrimidin-1(2H)-yl)-6-fluoro-1H-indazol-1-yl)piperidin-1-yl)methyl)piperidin-1-yl)pyridazine-3-carboxamide (EAR33)

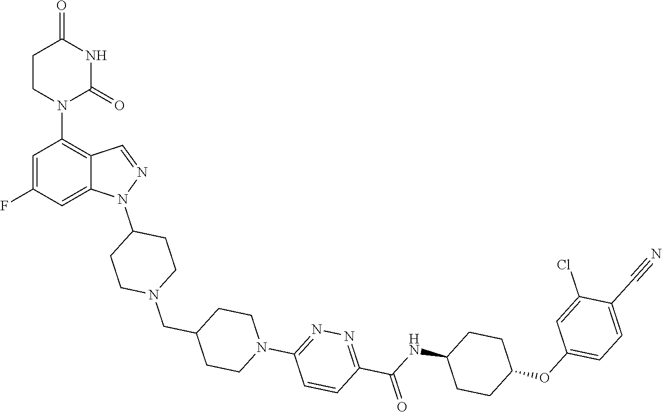 [SMILES: N#Cc1ccc(O[C@H]2CC[C@H](NC(=O)c3ccc(N4CCC(CN5CCC(n6ncc7c(N8CCC(=O)NC8=O)cc(F)cc76)CC5)CC4)nn3)CC2)cc1Cl]

N-((1r,4r)-4-(3-chloro-4-cyanophenoxy)cyclohexyl)-6-(4-formylpiperidin-1-yl)pyridazine-3-carboxamide (may be prepared as described in Description 95; 85 mg, 0.181 mmol), 1-(6-fluoro-1-(piperidin-4-yl)-1H-indazol-4-yl)dihydropyrimidine-2,4(1H,3H)-dione (may be prepared as described in Example CB10; 50 mg, 0.151 mmol) and DCM (2 mL) were mixed and stirred at room temperature for 10 min. Sodium triacetoxyborohydride (96 mg, 0.453 mmol) was added and the reaction mixture stirred at room temperature overnight (approximately 16 h). The reaction mixture was diluted with DCM, NaHCO₃ solution and brine, shaken vigorously, passed through a hydrophobic frit and the organic phase was evaporated. Purification by MDAP (high pH), evaporation of the fractions and drying in a vacuum oven at 40° C. for ca 24 h gave the title compound as a white powder (72 mg, 0.087 mmol, 58% yield). LCMS (high pH A): Rt=1.25 min, MH⁺=783.4.

### Example AR34

### 6-(4-((4-(6-Chloro-4-(2,4-dioxotetrahydropyrimidin-1(2H)-yl)-1H-indazol-1-yl)piperidin-1-yl)methyl)piperidin-1-yl)-N-((1r,4r)-4-(3-chloro-4-cyanophenoxy)cyclohexyl)pyridazine-3-carboxamide hydrochloride (EAR34)

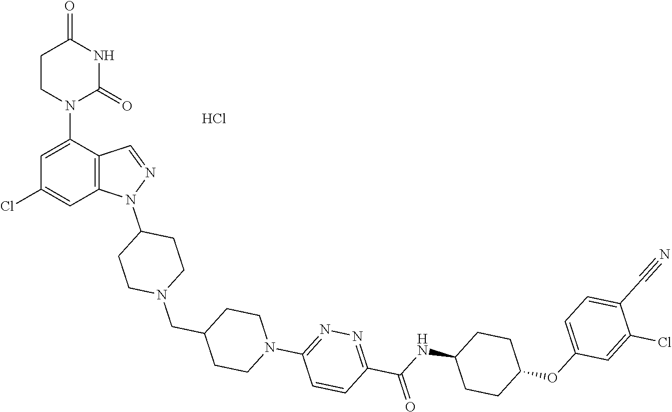 [SMILES: N#Cc1ccc(O[C@H]2CC[C@H](NC(=O)c3ccc(N4CCC(CN5CCC(n6ncc7c(N8CCC(=O)NC8=O)cc(Cl)cc76)CC5)CC4)nn3)CC2)cc1Cl ; Cl]

A mixture of 1-(6-chloro-1-(piperidin-4-yl)-1H-indazol-4-yl)dihydropyrimidine-2,4(1H,3H)-dione 2,2,2-trifluoroacetate (may be prepared as described in Example CB5; 52 mg, 0.113 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 63 mg, 0.135 mmol) in anhydrous DCM (1.0 mL) was treated with triethylamine (18 μL, 0.129 mmol) and allowed to stir in a stoppered vessel at room temperature for 10 min. Sodium triacetoxyborohydride (72 mg, 0.340 mmol) was added and the reaction mixture stirred in a stoppered vessel at room temperature for 4 h. Further N-((1r,4r)-4-(3-chloro-4-cyanophenoxy)cyclohexyl)-6-(4-formylpiperidin-1-yl)pyridazine-3-carboxamide (32 mg) was added and the reaction mixture stirred in a stoppered vessel at room temperature for 18 h. The reaction mixture was washed sequentially with saturated NaHCO₃ (aq) (1.0 mL) and brine (1.0 mL), and the organic layer passed through a hydrophobic frit. The filtrate was evaporated under a stream of nitrogen and the residue purified by MDAP (high pH). The appropriate fraction was evaporated in vacuo to give the free base (15.4 mg, 0.019 mmol). The solid was dissolved in 1,4-Dioxane (0.4 mL) and treated with 4 M HCl in 1,4-dioxane (8 μL, 0.032 mmol). The mixture was stirred in a stoppered at room temperature for 2 h. The mixture was evaporated under a stream of nitrogen and the solid dried in a vacuum oven to give the title compound as an off white solid (17 mg, 0.020 mmol). LCMS (high pH A): Rt=1.30 min, MH⁺=799, 801.

### Example AR35

### 1-(1-((1-(6-(((1r,4r)-4-(3-Chloro-4-cyanophenoxy)cyclohexyl)carbamoyl)pyridazin-3-yl)piperidin-4-yl)methyl)piperidin-4-yl)-4-(2,4-dioxotetrahydropyrimidin-1(2H)-yl)-N,N-dimethyl-1H-indole-6-carboxamide (EAR35)

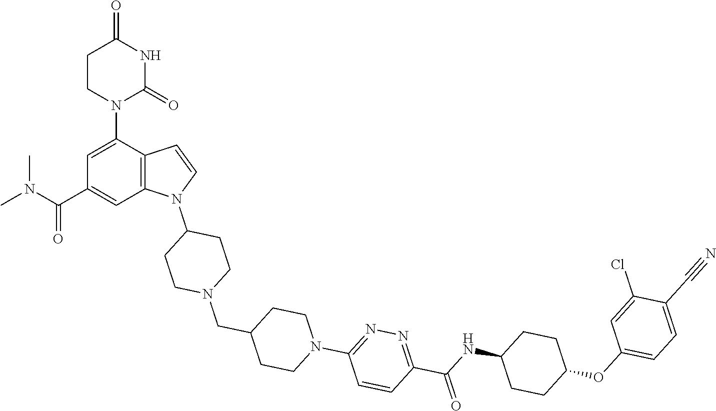 [SMILES: CN(C)C(=O)c1cc(N2CCC(=O)NC2=O)c2ccn(C3CCN(CC4CCN(c5ccc(C(=O)N[C@H]6CC[C@H](Oc7ccc(C#N)c(Cl)c7)CC6)nn5)CC4)CC3)c2c1]

A solution of 1-(1-((1-(6-(((1r,4r)-4-(3-chloro-4-cyanophenoxy)cyclohexyl)carbamoyl)pyridazin-3-yl)piperidin-4-yl)methyl)piperidin-4-yl)-4-(2,4-dioxo-3-((2-(trimethylsilyl)ethoxy)methyl)tetrahydropyrimidin-1(2H)-yl)-N,N-dimethyl-1H-indole-6-carboxamide (may be prepared as described in Description 171; 41 mg, 0.042 mmol) in DCM (0.50 mL) was treated with TFA (0.100 mL, 1.298 mmol) and the solution left to stand in a stoppered vessel at room temperature for 1.5 h. The mixture was evaporated under a stream of nitrogen and the residue dissolved in MeOH (0.50 mL) and DCM (0.50 mL). The solution was treated with 4 M ammonia in MeOH (1.2 mL, 4.80 mmol) and allowed to stir in a stoppered vessel at room temperature for 1.5 h. The mixture was evaporated under a stream of nitrogen and the residue partitioned between DCM (5 mL) and water (5 mL). The organic layer was passed through a hydrophobic frit and the filtrate evaporated onto Florisil. The Florisil-adsorbed material was purified on a 12 g silica cartridge using a gradient of 0-12.5% 2 M NH₃/MeOH in DCM over 14 column volumes. The appropriate fractions were combined and the solvent evaporated in vacuo. The material was dried in a vacuum oven to give the title compound as a white solid (22 mg, 0.026 mmol). LCMS (high pH A): Rt=1.17 min, MH⁺=835, 836, 837.

### Example AR36

### N-((1r,4r)-4-(3-Chloro-4-cyanophenoxy)cyclohexyl)-6-(3-((4-(4-(2,4-dioxotetrahydropyrimidin-1(2H)-yl)-1H-indazol-1-yl)piperidin-1-yl)methyl)piperidin-1-yl)pyridazine-3-carboxamide (EAR36)

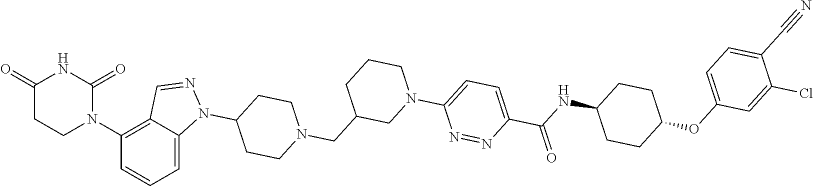 [SMILES: N#Cc1ccc(O[C@H]2CC[C@H](NC(=O)c3ccc(N4CCCC(CN5CCC(n6ncc7c(N8CCC(=O)NC8=O)cccc76)CC5)C4)nn3)CC2)cc1Cl]

1-(1-(Piperidin-4-yl)-1H-indazol-4-yl)dihydropyrimidine-2,4(1H,3H)-dione 2,2,2-trifluoroacetic acid salt (may be prepared as described in Example CB4; 90 mg, 0.211 mmol) was suspended in DCM (20 mL) at room temperature, then DIPEA (0.037 mL, 0.211 mmol) was added, followed by N-((1r,4r)-4-(3-chloro-4-cyanophenoxy)cyclohexyl)-6-(3-formylpiperidin-1-yl)pyridazine-3-carboxamide (may be prepared as described in Description 102; 90 mg, 0.192 mmol), and the mixture was stirred for 1 h. Then sodium triacetoxyborohydride (89 mg, 0.421 mmol) was added, and the mixture stirred for 2 h. Sodium bicarbonate solution (20 mL) was added and the mixture stirred for 20 min, then the phases were separated and the organic layer dried and evaporated in vacuo to give a colourless solid. The crude compound was dissolved in DCM and loaded onto a 24 g silica column, then eluted with 0-20% 2 M MeOH ammonia/DCM and product-containing fractions evaporated in vacuo to give the title compound as a colourless solid (108 mg, 0.141 mmol, 67% yield). LCMS (high pH A): Rt=1.27 min, MH⁺=765, 767.

### Examples AR37-AR42

### General Procedure

A mixture of 1-(1-(piperidin-4-yl)-1H-indazol-4-yl)dihydropyrimidine-2,4(1H,3H)-dione 2,2,2-trifluoroacetate (may be prepared as described in Example CB4; 50 mg, 0.117 mmol) and the appropriate aldehyde (0.117 mmol) in DCM (1.0 mL) was treated with triethylamine (0.018 mL, 0.129 mmol) and stirred in a stoppered vessel at room temperature for 10 min. Sodium triacetoxyborohydride (74 mg, 0.349 mmol) was added and the mixture stirred in a stoppered vessel at room temperature for 18 h. Further Sodium triacetoxyborohydride (74 mg, 0.349 mmol) was added and the mixture stirred in a stoppered vessel at room temperature for 24 h. The reaction was treated with saturated NaHCO₃ (aq) (2.0 mL) and extracted with DCM (2.0 mL). The organic extract was passed through a hydrophobic frit and the filtrate evaporated under a stream of nitrogen. The residue was purified using MDAP (high pH). The solid was dissolved in 1,4-dioxane (0.50 mL) and treated with the appropriate amount of 4 M HCl in dioxane. The mixtures were stirred in stoppered vessels at room temperature for 3 h then evaporated under a stream of nitrogen. The solids were dried in a vacuum oven to give the compounds below:

| | | | Volume |
|---|---|---|---|
| | | | 4M HCl |
| | | | in |
| | | | dioxane |
| | | | (μL); |
| Ex- | Name | Alde- | Mass; |
| ample | Analytical | hyde | yield |
| AR37 | 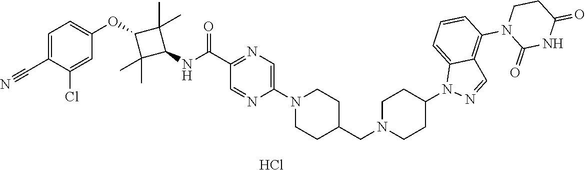 [SMILES: CC1(C)[C@H](NC(=O)c2cnc(N3CCC(CN4CCC(n5ncc6c(N7CCC(=O)NC7=O)cccc65)CC4)CC3)cn2)C(C)(C)[C@H]1Oc1ccc(C#N)c(Cl)c1 ; Cl] | D92 | 20 μL 41.1 mg, 40% |
| | N-((1r,3r)-3-(3-Chloro-4-cyanophenoxy)-2,2,4,4- | | |
| | tetramethylcyclobutyl)-5-(4-((4-(4-(2,4- | | |
| | dioxotetrahydropyrimidin-1(2H)-yl)-1H-indazol-1-yl)piperidin- | | |
| | 1-yl)methyl)piperidin-1-yl)pyrazine-2-carboxamide | | |
| | hydrochloride | | |
| | LCMS (high pH A): Rt = 1.41 min, MH⁺ = 793 | | |
| AR38 | 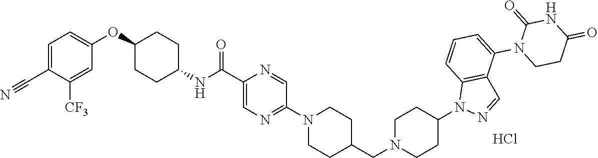 [SMILES: N#Cc1ccc(O[C@H]2CC[C@H](NC(=O)c3cnc(N4CCC(CN5CCC(n6ncc7c(N8CCC(=O)NC8=O)cccc76)CC5)CC4)cn3)CC2)cc1C(F)(F)F ; Cl] | D98 | 19 μL 35.8 mg, 35% |
| | N-((1r,4r)-4-(4-Cyano-3- | | |
| | (trifluoromethyl)phenoxy)cyclohexyl)-5-(4-((4-(4-(2,4- | | |
| | dioxotetrahydropyrimidin-1(2H)-yl)-1H-indazol-1-yl)piperidin- | | |
| | 1-yl)methyl)piperidin-1-yl)pyrazine-2-carboxamide | | |
| | hydrochloride | | |
| | LCMS (high pH A): Rt = 1.28 min, MH⁺ = 799 | | |
| AR39 | 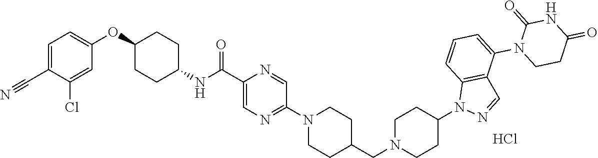 [SMILES: N#Cc1ccc(O[C@H]2CC[C@H](NC(=O)c3cnc(N4CCC(CN5CCC(n6ncc7c(N8CCC(=O)NC8=O)cccc76)CC5)CC4)cn3)CC2)cc1Cl ; Cl] | D99 | 8 μL 19.9 mg, 20% |
| | N-((1r,4r)-4-(3-Chloro-4-cyanophenoxy)cyclohexyl)-5-(4-((4- | | |
| | (4-(2,4-dioxotetrahydropyrimidin-1(2H)-yl)-1H-indazol-1- | | |
| | yl)piperidin-1-yl)methyl)piperidin-1-yl)pyrazine-2- | | |
| | carboxamide hydrochloride | | |
| | LCMS (high pH A): Rt = 1.26 min, MH⁺ = 765 | | |
| AR40 | 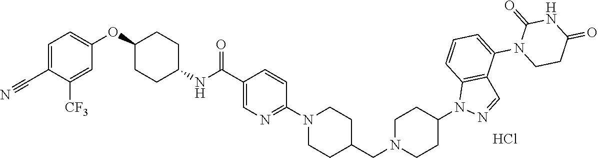 [SMILES: N#Cc1ccc(O[C@H]2CC[C@H](NC(=O)c3ccc(N4CCC(CN5CCC(n6ncc7c(N8CCC(=O)NC8=O)cccc76)CC5)CC4)nc3)CC2)cc1C(F)(F)F ; Cl] | D101 | 22 μL 46.5 mg, 45% |
| | N-((1r,4r)-4-(4-Cyano-3- | | |
| | (trifluoromethyl)phenoxy)cyclohexyl)-6-(4-((4-(4-(2,4- | | |
| | dioxotetrahydropyrimidin-1(2H)-yl)-1H-indazol-1-yl)piperidin- | | |
| | 1-yl)methyl)piperidin-1-yl)nicotinamide hydrochloride | | |
| | LCMS (high pH A): Rt = 1.23 min, MH⁺ = 798 | | |
| AR41 | 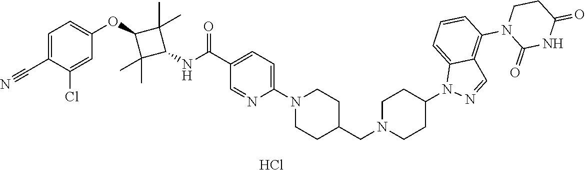 [SMILES: CC1(C)[C@H](NC(=O)c2ccc(N3CCC(CN4CCC(n5ncc6c(N7CCC(=O)NC7=O)cccc65)CC4)CC3)nc2)C(C)(C)[C@H]1Oc1ccc(C#N)c(Cl)c1 ; Cl] | D96 | 18 μL 37.4 mg, 37% |
| | N-((1r,3r)-3-(3-Chloro-4-cyanophenoxy)-2,2,4,4- | | |
| | tetramethylcyclobutyl)-6-(4-((4-(4-(2,4- | | |
| | dioxotetrahydropyrimidin-1(2H)-yl)-1H-indazol-1-yl)piperidin- | | |
| | 1-yl)methyl)piperidin-1-yl)nicotinamide hydrochloride | | |
| | LCMS (high pH A): Rt = 1.35 min, MH⁺ = 792 | | |
| AR42 | 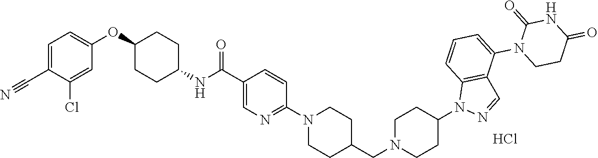 [SMILES: N#Cc1ccc(O[C@H]2CC[C@H](NC(=O)c3ccc(N4CCC(CN5CCC(n6ncc7c(N8CCC(=O)NC8=O)cccc76)CC5)CC4)nc3)CC2)cc1Cl ; Cl] | D94 | 21 μL 29 mg, 29% |
| | N-((1r,4r)-4-(3-Chloro-4-cyanophenoxy)cyclohexyl)-6-(4-((4- | | |
| | (4-(2,4-dioxotetrahydropyrimidin-1(2H)-yl)-1H-indazol-1- | | |
| | yl)piperidin-1-yl)methyl)piperidin-1-yl)nicotinamide | | |
| | hydrochloride | | |
| | LCMS (high pH A): Rt = 1.21 min, MH⁺ = 764 | | |

### Example AR43

### N-((1r,4r)-4-(3-Chloro-4-cyanophenoxy)cyclohexyl)-6-(4-((4-(4-(2,4-dioxotetrahydropyrimidin-1(2H)-yl)-1H-indazol-1-yl)piperidin-1-yl)methyl)piperidin-1-yl)pyridazine-3-carboxamide (EAR43)

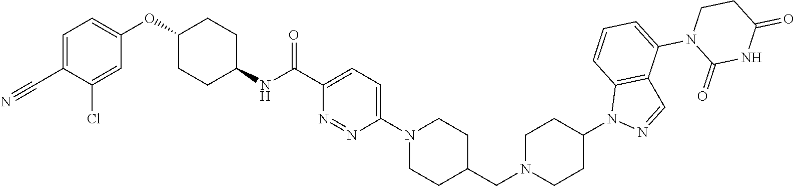 [SMILES: N#Cc1ccc(O[C@H]2CC[C@H](NC(=O)c3ccc(N4CCC(CN5CCC(n6ncc7c(N8CCC(=O)NC8=O)cccc76)CC5)CC4)nn3)CC2)cc1Cl]

A microwave vial was charged with N-((1r,4r)-4-(3-chloro-4-cyanophenoxy)cyclohexyl)-6-(4-formylpiperidin-1-yl)pyridazine-3-carboxamide (may be prepared as described in Description 95; 50 mg, 0.107 mmol), 1-(1-(piperidin-4-yl)-1H-indazol-4-yl)dihydropyrimidine-2,4(1H,3H)-dione (may be prepared as described in Example CB18; 34 mg, 0.109 mmol), 2-picoline borane (23 mg, 0.215 mmol) and titanium (IV) isopropoxide (0.032 mL, 0.109 mmol) in THF (1 mL) and sealed. The reaction mixture was purged with nitrogen for 10 min then stirred at 50° C. for 1 h. The reaction mixture was diluted with saturated aqueous sodium hydrogen carbonate (7 mL) and extracted with EtOAc (3×10 mL). The organic layers were combined, passed through a hydrophobic frit, and the solvent removed in vacuo. The residue was purified by normal phase column chromatography (0-25% ethanol in DCM, 24 g silica, 14 column volumes) to give the title compound as a white powder (32 mg, 0.042 mmol, 39% yield). LCMS (formic A): Rt=0.82 min, MH⁺=765, 767

### Example AR44

### N-((1r,4r)-4-(4-Cyano-3-(trifluoromethyl)phenoxy)cyclohexyl)-6-(4-((4-(4-(2,4-dioxotetrahydropyrimidin-1(2H)-yl)-1H-indazol-1-yl)piperidin-1-yl)methyl)piperidin-1-yl)pyridazine-3-carboxamide (EAR44)

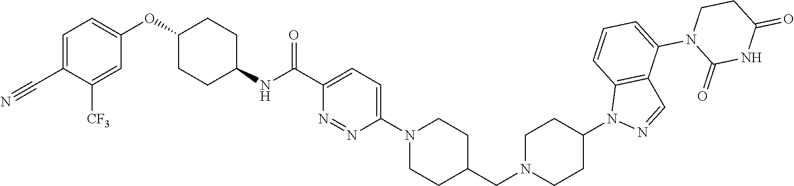 [SMILES: N#Cc1ccc(O[C@H]2CC[C@H](NC(=O)c3ccc(N4CCC(CN5CCC(n6ncc7c(N8CCC(=O)NC8=O)cccc76)CC5)CC4)nn3)CC2)cc1C(F)(F)F]

A microwave vial was charged with N-((1r,4r)-4-(4-cyano-3-(trifluoromethyl)phenoxy)cyclohexyl)-6-(4-formylpiperidin-1-yl)pyridazine-3-carboxamide (may be prepared as described in Description 97; 100 mg, 0.120 mmol), 1-(1-(piperidin-4-yl)-1H-indazol-4-yl)dihydropyrimidine-2,4(1H,3H)-dione (may be prepared as described in Example CB18; 37.9 mg, 0.121 mmol) and DCM (1424 μL). To the stirred mixture was added sodium triacetoxyborohydride (76 mg, 0.359 mmol) and the mixture stirred at room temperature for 30 min and left to stand for 1 h. The reaction mixture was quenched with saturated aqueous sodium bicarbonate solution (20 mL) and extracted with DCM (3×25 mL). The combined organic extracts were dried by passing through a hydrophobic frit and the solvent removed in vacuo to give a gummy yellow solid, which was dissolved in 1:1 DMSO:MeOH (1 mL) and purified by MDAP (high pH) in 2 portions. The appropriate fractions were collected and the solvent removed under a stream of nitrogen to give the title compound as a white solid (59 mg, 0.074 mmol, 62% yield). LCMS (high pH A): Rt=1.24 min, MH⁺=799

### Example AR45

### N-((1r,4r)-4-(4-Cyano-3-(trifluoromethyl)phenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)pyridazine-3-carboxamide (EAR45)

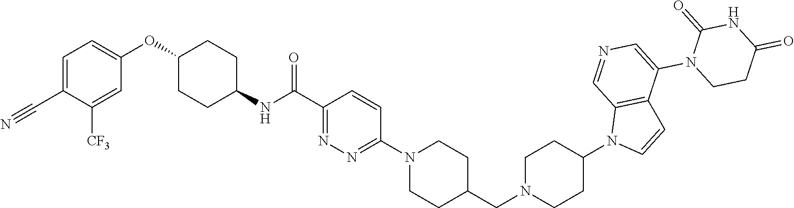 [SMILES: N#Cc1ccc(O[C@H]2CC[C@H](NC(=O)c3ccc(N4CCC(CN5CCC(n6ccc7c(N8CCC(=O)NC8=O)cncc76)CC5)CC4)nn3)CC2)cc1C(F)(F)F]

A microwave vial was charged with N-((1r,4r)-4-(4-cyano-3-(trifluoromethyl)phenoxy)cyclohexyl)-6-(4-formylpiperidin-1-yl)pyridazine-3-carboxamide (may be prepared as described in Description 97; 100 mg, 0.120 mmol), 1-(1-(piperidin-4-yl)-1H-pyrrolo[2,3-c]pyridin-4-yl)dihydropyrimidine-2,4(1H,3H)-dione (may be prepared as described in Example CB6; 37.9 mg, 0.121 mmol) and DCM (1424 μL). To the stirred mixture was added sodium triacetoxyborohydride (76 mg, 0.359 mmol), and the mixture stirred at room temperature for 30 min and left to stand for 1 h. The reaction mixture was quenched with saturated aqueous sodium bicarbonate solution (20 mL) and extracted with DCM (3×25 mL). The combined organic extracts were dried by passing through a hydrophobic frit and the solvent removed in vacuo to give a gummy yellow solid which was dissolved in 1:1 DMSO:MeOH (1 mL) and purified by MDAP (high pH) in 2 batches. Appropriate fractions were collected and the solvent removed under a stream of nitrogen to give the title compound as a white solid (52.6 mg, 0.066 mmol, 55% yield). LCMS (high pH A): Rt=1.16 min, MH⁺=799.

### Example AR46

### N-((1r,4r)-4-(4-Cyano-3-(trifluoromethyl)phenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)pyridazine-3-carboxamide hydrochloride (EAR46)

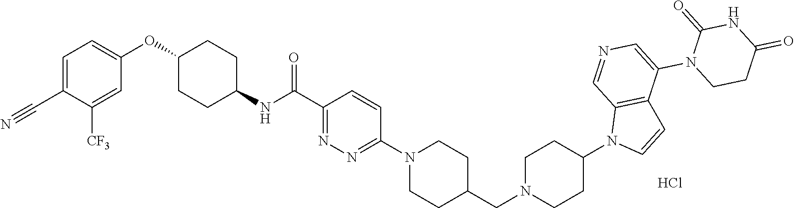 [SMILES: N#Cc1ccc(O[C@H]2CC[C@H](NC(=O)c3ccc(N4CCC(CN5CCC(n6ccc7c(N8CCC(=O)NC8=O)cncc76)CC5)CC4)nn3)CC2)cc1C(F)(F)F ; Cl]

To N-((1r,4r)-4-(4-cyano-3-(trifluoromethyl)phenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)pyridazine-3-carboxamide (may be prepared as described in Example AR45; 37.68 mg, 0.047 mmol) suspended in 1,4-dioxane (200 μL) and stirred at room temperature was added HCl (4 M in Dioxane) (45.4 μL, 0.182 mmol). The reaction mixture was stirred at room temperature for 4 h then dried under a stream of nitrogen for 4 h, then further dried in a vacuum oven overnight to give the title compound as a pale yellow solid (37 mg, 0.044 mmol, 94% yield). LCMS (high pH A): Rt=1.16 min, MH⁺=799.

### Example AR47

### N-((1r,4r)-4-(3-Chloro-4-cyanophenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)pyridazine-3-carboxamide (EAR47)

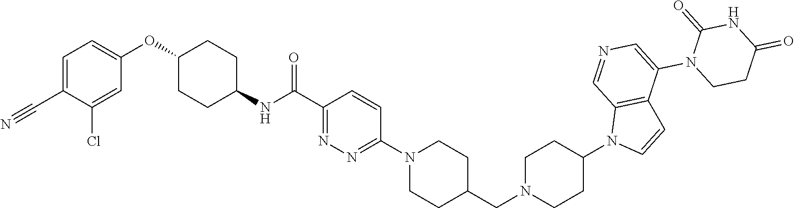 [SMILES: N#Cc1ccc(O[C@H]2CC[C@H](NC(=O)c3ccc(N4CCC(CN5CCC(n6ccc7c(N8CCC(=O)NC8=O)cncc76)CC5)CC4)nn3)CC2)cc1Cl]

A microwave vial was charged with N-((1r,4r)-4-(3-chloro-4-cyanophenoxy)cyclohexyl)-6-(4-formylpiperidin-1-yl)pyridazine-3-carboxamide (may be prepared as described in Description 95; 101.9 mg, 0.218 mmol), 1-(1-(piperidin-4-yl)-1H-pyrrolo[2,3-c]pyridin-4-yl)dihydropyrimidine-2,4(1H,3H)-dione (may be prepared as described in Example CB6; 69 mg, 0.220 mmol) and DCM (2.6 mL). To the stirred mixture was added sodium triacetoxyborohydride (138 mg, 0.653 mmol) and the mixture stirred at room temperature for 30 min. The reaction mixture was quenched with saturated aqueous sodium bicarbonate solution (20 mL) and extracted with DCM (3×25 mL). The combined organic extracts were dried by passing through a hydrophobic frit and the solvent removed in vacuo to give a gummy yellow solid. 1:1 DMSO:MeOH (1 mL) was added, forming a suspension. The mixture was left to sit for 1.5 h, filtered and the solids washed with MeOH (2 mL) and dried under vacuum to give the title compound as a white solid (103 mg, 0.135 mmol, 62% yield). LCMS (high pH A): Rt=1.14 min, MH⁺=765, 767.

### Example AR48

### N-((1r,4r)-4-(3-Chloro-4-cyanophenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)pyridazine-3-carboxamide hydrochloride (EAR48)

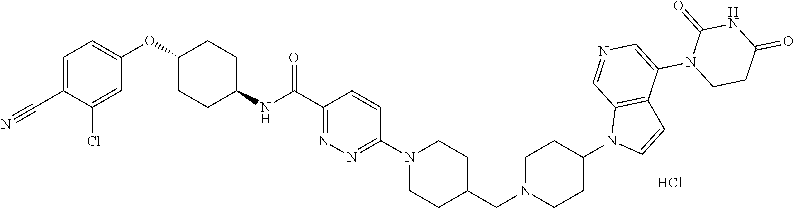 [SMILES: N#Cc1ccc(O[C@H]2CC[C@H](NC(=O)c3ccc(N4CCC(CN5CCC(n6ccc7c(N8CCC(=O)NC8=O)cncc76)CC5)CC4)nn3)CC2)cc1Cl ; Cl]

To a suspension of N-((1r,4r)-4-(3-chloro-4-cyanophenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)pyridazine-3-carboxamide (may be prepared as described in Example AR47; 49.93 mg, 0.065 mmol) in 1,4-Dioxane (200 μL) stirred at room temperature was added HCl (4 M in Dioxane) (16.31 μL, 0.065 mmol). The reaction mixture was stirred at room temperature for 2 h. The solvent was removed under a stream of nitrogen to give a white solid. This material was resuspended in 1,4-dioxane (500 μL) and HCl (4 M in dioxane) (24.47 μL, 0.098 mmol) added, and stirred for 2 h. The solvent was removed under a stream of nitrogen to give the title compound as a white solid (52.7 mg, 0.066 mmol, 101% yield). LCMS (high pH A): Rt=1.15 min, MH⁺=765, 767.

### Example AR49

### N-((1r,4r)-4-(3-Chloro-4-cyanophenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)nicotinamide (AR49)

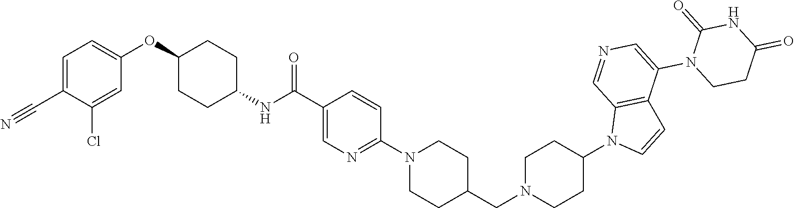 [SMILES: N#Cc1ccc(O[C@H]2CC[C@H](NC(=O)c3ccc(N4CCC(CN5CCC(n6ccc7c(N8CCC(=O)NC8=O)cncc76)CC5)CC4)nc3)CC2)cc1Cl]

To a mixture of 1-(1-(piperidin-4-yl)-1H-pyrrolo[2,3-c]pyridin-4-yl)dihydropyrimidine-2,4(1H,3H)-dione (may be prepared as described in Example CB6; 50 mg, 0.160 mmol) and N-((1r,4r)-4-(3-chloro-4-cyanophenoxy)cyclohexyl)-6-(4-formylpiperidin-1-yl)nicotinamide (may be prepared as described in Description 94; 76 mg, 0.163 mmol) in anhydrous DCM (2 mL) was added sodium triacetoxyborohydride (101 mg, 0.479 mmol), and the reaction mixture stirred in a stoppered vessel at room temperature for 15 h. The reaction mixture was washed sequentially with saturated NaHCO₃ (aq (2 mL) and brine (2 mL)—some gummy solid was formed and this was retained in the organic layer which was evaporated in vacuo. The residue was suspended in DMSO (2 mL) and filtered. The filtrate was split into two batches and each purified by MDAP (high pH). The appropriate fractions were combined and the solvent evaporated in vacuo to give the title compound as a white solid (42 mg, 0.055 mmol). LCMS (high pH A): Rt=1.13 min, MH⁺=764, 765, 766.

### Example AR50

### N-((1r,4r)-4-(3-Chloro-4-cyanophenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)nicotinamide hydrochloride (EAR50)

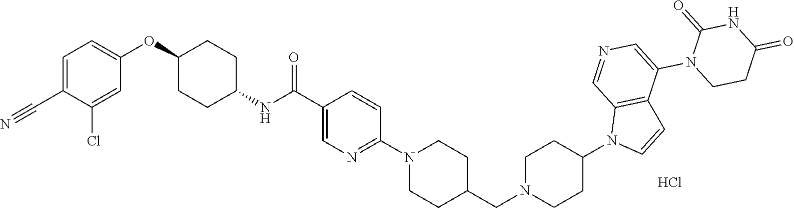 [SMILES: N#Cc1ccc(O[C@H]2CC[C@H](NC(=O)c3ccc(N4CCC(CN5CCC(n6ccc7c(N8CCC(=O)NC8=O)cncc76)CC5)CC4)nc3)CC2)cc1Cl ; Cl]

A solution of N-((1r,4r)-4-(3-chloro-4-cyanophenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)nicotinamide (may be prepared as described in Example AR49; 27 mg, 0.035 mmol) in 1,4-dioxane (0.50 mL) was treated with 4 M HCl in dioxane (13 μL, 0.053 mmol), and the mixture stirred at room temperature in a stoppered vessel for 2 h. The mixture was evaporated under a stream of nitrogen and the solid dried in a vacuum oven for 16 h to give the title compound as a white solid (26 mg, 0.032 mmol). LCMS (high pH A): Rt=1.12 min, MH⁺=764, 765, 766.

### Example AR51

### N-((1r,4r)-4-(3-Chloro-4-cyanophenoxy)cyclohexyl)-6-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)pyridazine-3-carboxamide (EAR51)

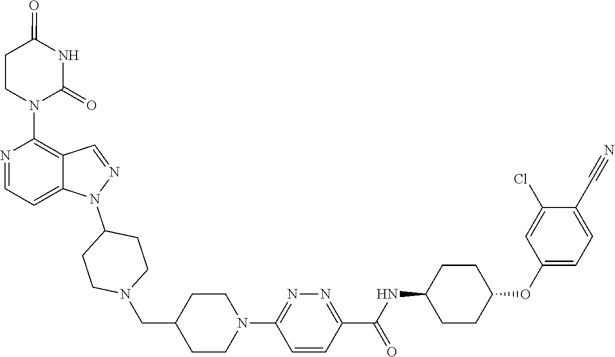 [SMILES: N#Cc1ccc(O[C@H]2CC[C@H](NC(=O)c3ccc(N4CCC(CN5CCC(n6ncc7c(N8CCC(=O)NC8=O)nccc76)CC5)CC4)nn3)CC2)cc1Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 89 mg, 0.191 mmol), 1-(1-(piperidin-4-yl)-1H-pyrazolo[4,3-c]pyridin-4-yl)dihydropyrimidine-2,4(1H,3H)-dione (may be prepared as described in Example CB7; 50 mg, 0.159 mmol) and DCM (2 mL) were mixed and stirred at room temperature for 10 min. Sodium triacetoxyborohydride (101 mg, 0.477 mmol) was added and the reaction mixture stirred overnight (approximately 16 h). The reaction mixture was diluted with DCM and NaHCO₃ and brine, shaken vigorously and passed through a hydrophobic frit and concentrated under a stream of nitrogen. Purification by MDAP (high pH) gave the title compound as a slightly off-white powder (75 mg, 0.093 mmol, 59% yield). LCMS (high pH A): Rt=1.19 min, MH⁺=766.4.

### Example AR52

### N-((1r,4r)-4-(3-Chloro-4-cyanophenoxy)cyclohexyl)-6-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyridazine-3-carboxamide (EAR52)

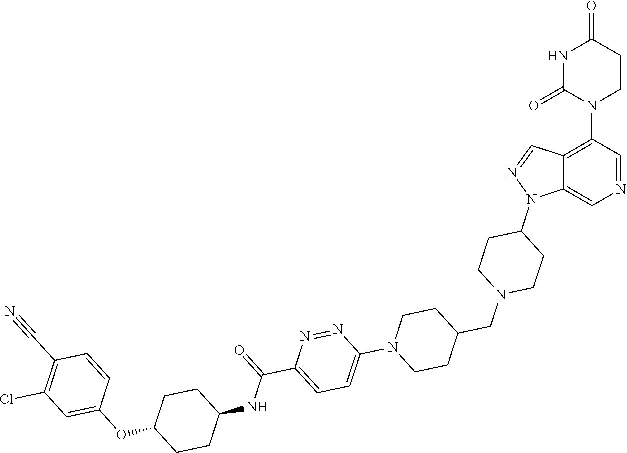 [SMILES: N#Cc1ccc(O[C@H]2CC[C@H](NC(=O)c3ccc(N4CCC(CN5CCC(n6ncc7c(N8CCC(=O)NC8=O)cncc76)CC5)CC4)nn3)CC2)cc1Cl]

A microwave vial was charged with N-((1r,4r)-4-(3-chloro-4-cyanophenoxy)cyclohexyl)-6-(4-formylpiperidin-1-yl)pyridazine-3-carboxamide (may be prepared as described in Description 95; 60 mg, 0.128 mmol), 1-(1-(piperidin-4-yl)-1H-pyrazolo[3,4-c]pyridin-4-yl)dihydropyrimidine-2,4(1H,3H)-dione (may be prepared as described in Example CB8; 40.7 mg, 0.129 mmol) and DCM (1.5 mL). To the stirred mixture was added sodium triacetoxyborohydride (82 mg, 0.385 mmol) and the mixture stirred at room temperature for 30 min. The reaction mixture was quenched with saturated aqueous sodium bicarbonate solution (20 mL) and extracted with DCM (3×25 mL). The combined organic extracts were dried by passing through a hydrophobic frit and the solvent removed in vacuo to give a gummy yellow solid. This was treated with 1:1 DMSO:MeOH (0.5 mL) forming a suspension. The mixture was filtered and the solids washed with MeOH (2 mL) and dried under vacuum. This was treated with MeOH (0.5 mL) and the suspension sonicated. The solids were collected by filtration, washed with MeOH (2 mL) and dried under vacuum to give a white solid. The filtrate from the first filtration was diluted with minimal MeOH and the resulting mixture was sonicated to get a fine solid suspension. The solids were collected by filtration and dried under vacuum to give a white solid. The two white solids were combined to give the title compound as a white solid (39.6 mg, 0.052 mmol, 40% yield). LCMS: (high pH) Rt=1.14 mins, MH⁺=766.3, 768.3.

### Example AR53

### N-((1r,4r)-4-(3-Chloro-4-cyanophenoxy)cyclohexyl)-6-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyridazine-3-carboxamide hydrochloride (EAR53)

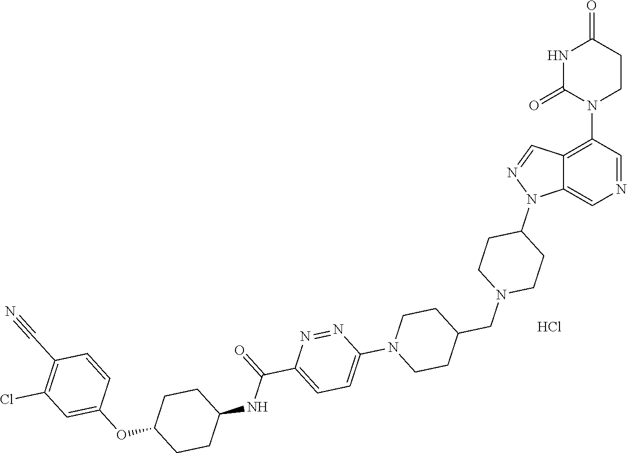 [SMILES: N#Cc1ccc(O[C@H]2CC[C@H](NC(=O)c3ccc(N4CCC(CN5CCC(n6ncc7c(N8CCC(=O)NC8=O)cncc76)CC5)CC4)nn3)CC2)cc1Cl ; Cl]

To a suspension of N-((1r,4r)-4-(3-chloro-4-cyanophenoxy)cyclohexyl)-6-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyridazine-3-carboxamide (may be prepared as described in Example AR52; 32.52 mg, 0.042 mmol) in 1,4-Dioxane (200 μL) stirred at room temperature was added HCl (4 M in dioxane) (40.7 μL, 0.163 mmol). The reaction mixture was stirred at room temperature for 2 h. The solvent was removed under a stream of nitrogen and further dried in a vacuum oven to give the title compound as a yellow solid. (31.4 mg, 0.039 mmol, 92% yield). LCMS: (high pH) Rt=1.14 mins, MH⁺=766.2, 768.2.

### Example AR54

### N-((1r,4r)-4-(3-Chloro-4-cyanophenoxy)cyclohexyl)-4-(4-(4-(2,4-dioxotetrahydropyrimidin-1(2H)-yl)-1H-indol-1-yl)piperidin-1-yl)benzamide (EAR54)

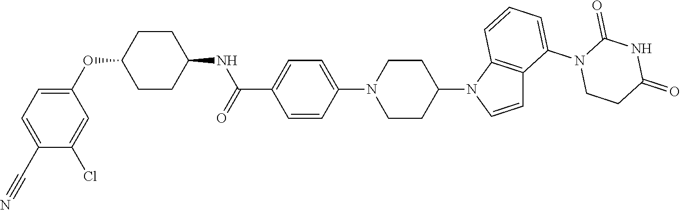 [SMILES: N#Cc1ccc(O[C@H]2CC[C@H](NC(=O)c3ccc(N4CCC(n5ccc6c(N7CCC(=O)NC7=O)cccc65)CC4)cc3)CC2)cc1Cl]

A mixture of 4-(4-(4-(2,4-dioxotetrahydropyrimidin-1(2H)-yl)-1H-indol-1-yl)piperidin-1-yl)benzoic acid trifluoroacetic acid salt (may be prepared as described in Example CBL5; 24 mg, 0.044 mmol), HATU (37 mg, 0.097 mmol) and DIPEA (0.025 mL, 0.143 mmol) were dissolved in DMF (0.5 mL) and allowed to stir at room temperature for 15 min. To this 4-(((1r,4r)-4-aminocyclohexyl)oxy)-2-chlorobenzonitrile (may be prepared as described in Description 46; 24 mg, 0.096 mmol) was then added and the mixture allowed to continue stirring in a stopped vessel at room temperature for 3.5 h. The reaction mixture was then diluted with water (10 mL) before being extracted with EtOAc (30 mL×2) and washed with Brine (20 mL). The organic layer was then passed through a hydrophobic frit before being evaporated under vacuo. Resulting solid was purified by MDAP (high pH). The appropriate fractions were combined and the solvent removed by rotary evaporation to give a partially pure white solid which was purified again by MDAP (formic). The appropriate fractions were combined and the solvent removed by rotary evaporation to give the title compound as a white solid (3.6 mg, 5.41 μmol). LCMS (formic A): Rt=1.20 min, MH⁺=665.

### Example AR55

### N-((1r,3r)-3-(3-Chloro-4-cyanophenoxy)-2,2,4,4-tetramethylcyclobutyl)-6-(4-((4-(4-(2,4-dioxotetrahydropyrimidin-1(2H)-yl)-1H-indol-1-yl)piperidin-1-yl)methyl)piperidin-1-yl)pyridazine-3-carboxamide (EAR55)

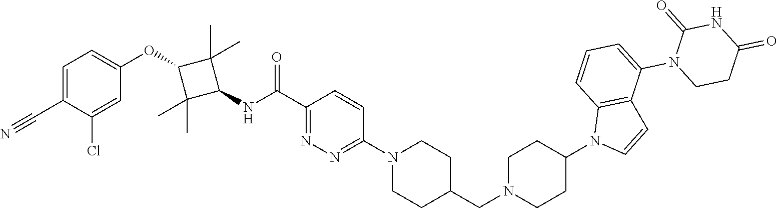 [SMILES: CC1(C)[C@H](NC(=O)c2ccc(N3CCC(CN4CCC(n5ccc6c(N7CCC(=O)NC7=O)cccc65)CC4)CC3)nn2)C(C)(C)[C@H]1Oc1ccc(C#N)c(Cl)c1]

(1-(6-(((1r,3r)-3-(3-Chloro-4-cyanophenoxy)-2,2,4,4-tetramethylcyclobutyl)carbamoyl)pyridazin-3-yl)piperidin-4-yl)methyl methanesulfonate (may be prepared as described in Description 110; 40 mg, 0.069 mmol), 1-(1-(piperidin-4-yl)-1H-indol-4-yl)dihydropyrimidine-2,4(1H,3H)-dione (may be prepared as described in Example CB1; 21.69 mg, 0.069 mmol) and DIPEA (0.036 mL, 0.208 mmol) were dissolved in DMF (1 mL) and the mixture stirred at room temperature for 2 h, then heated to 100° C. for 2 h. The reaction was purified by MDAP (high pH) to give the title compound as a colourless solid (2.45 mg, 3.09 μmol, 4% yield). LCMS (high pH A): Rt=1.44 min, MH⁺=790, 792.

### Example AR56

### N-((1r,3r)-3-(3-Chloro-4-cyanophenoxy)-2,2,4,4-tetramethylcyclobutyl)-6-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)pyridazine-3-carboxamide (EAR56)

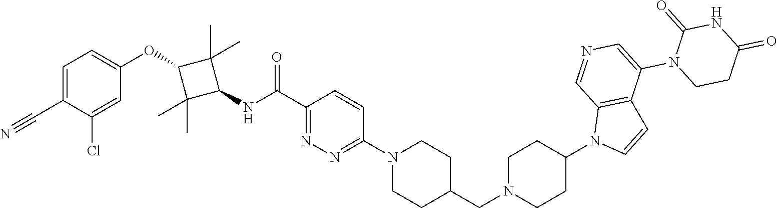 [SMILES: CC1(C)[C@H](NC(=O)c2ccc(N3CCC(CN4CCC(n5ccc6c(N7CCC(=O)NC7=O)cncc65)CC4)CC3)nn2)C(C)(C)[C@H]1Oc1ccc(C#N)c(Cl)c1]

(1-(6-(((1r,3r)-3-(3-Chloro-4-cyanophenoxy)-2,2,4,4-tetramethylcyclobutyl)carbamoyl)pyridazin-3-yl)piperidin-4-yl)methyl methanesulfonate (may be prepared as described in Description 110; 40 mg, 0.069 mmol), 1-(1-(piperidin-4-yl)-1H-pyrrolo[2,3-c]pyridin-4-yl)dihydropyrimidine-2,4(1H,3H)-dione (may be prepared as described in Example CB6; 21.76 mg, 0.069 mmol) and DIPEA (0.036 mL, 0.208 mmol) were dissolved in DMF (1 mL) and the mixture stirred at room temperature for 2 h, then heated to 100° C. for 2 h. The reaction was purified MDAP (high pH) to give the title compound as a brown solid (5.12 mg, 6.45 μmol, 9% yield). LCMS (high pH A): Rt=1.30 min, MH⁺=791, 793.

### Example AR57

### N-((1r,4r)-4-(3-Chloro-4-cyanophenoxy)cyclohexyl)-6-(4-(2-(4-(4-(2,4-dioxotetrahydropyrimidin-1(2H)-yl)-1H-indol-1-yl)piperidin-1-yl)ethyl)piperidin-1-yl)pyridazine-3-carboxamide hydrochloride (EAR57)

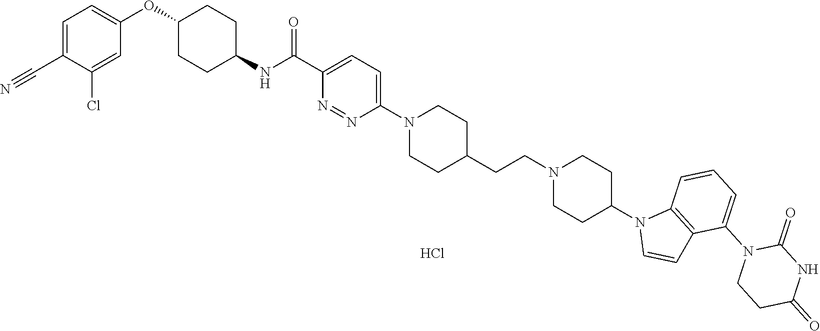 [SMILES: N#Cc1ccc(O[C@H]2CC[C@H](NC(=O)c3ccc(N4CCC(CCN5CCC(n6ccc7c(N8CCC(=O)NC8=O)cccc76)CC5)CC4)nn3)CC2)cc1Cl ; Cl]

A solution of 1-(1-(piperidin-4-yl)-1H-indol-4-yl)dihydropyrimidine-2,4(1H,3H)-dione (may be prepared as described in Example CB1; 30 mg, 0.096 mmol) in anhydrous DMF (0.7 mL) was added to 6-(4-(2-bromoethyl)piperidin-1-yl)-N-((1r,4r)-4-(3-chloro-4-cyanophenoxy)cyclohexyl)pyridazine-3-carboxamide (may be prepared as described in Description 112; 55 mg, 0.101 mmol), and triethylamine (26.8 μL, 0.192 mmol) was added. The vessel was sealed and the mixture stirred at room temperature 22 h. The sealed vessel was stirred at 50° C. for 3 h. The mixture was allowed to cool to room temperature and purified by MDAP (high pH). The fractions were evaporated under a stream of nitrogen to give N-((1r,4r)-4-(3-chloro-4-cyanophenoxy)cyclohexyl)-6-(4-(2-(4-(4-(2,4-dioxotetrahydropyrimidin-1(2H)-yl)-1H-indol-1-yl)piperidin-1-yl)ethyl)piperidin-1-yl)pyridazine-3-carboxamide (9.2 mg, 0.012 mmol), 4.2 mg of which was used to prepare a 10 mM in DMSO stock solution for testing. Solubility was poor in DMSO, so the suspension was evaporated under a stream of nitrogen and combined with the 4.8 mg stock solid. The solids were suspended in MeOH (0.5 mL) and treated with 2 M aqueous HCl (7.0 μL, 0.014 mmol). The mixture was stirred at room temperature for 1 h, filtered and the filtrate evaporated under a stream of nitrogen to give the title compound as an off white solid (4.5 mg, 5.52 μmol). LCMS (high pH A): Rt=1.28 min, MH⁺=778, 780.

### Example AR58

### N-((1r,4r)-4-(3-Chloro-4-cyanophenoxy)cyclohexyl)-6-(4-(2-(4-(4-(2,4-dioxotetrahydropyrimidin-1(2H)-yl)-1-isopropyl-1H-indole-6-carbonyl)piperazin-1-yl)ethyl)piperidin-1-yl)pyridazine-3-carboxamide hydrochloride (EAR58)

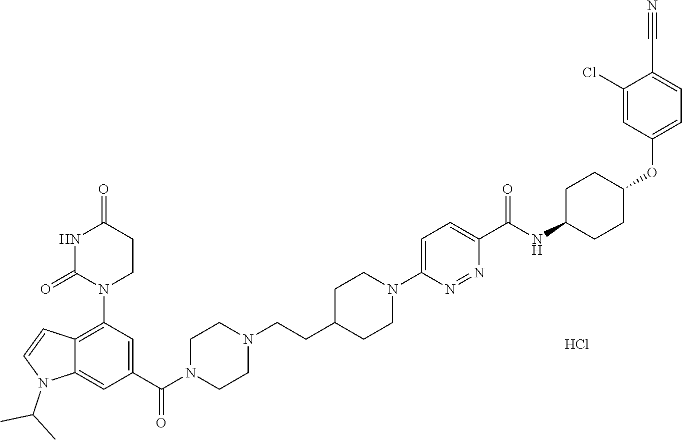 [SMILES: CC(C)n1ccc2c(N3CCC(=O)NC3=O)cc(C(=O)N3CCN(CCC4CCN(c5ccc(C(=O)N[C@H]6CC[C@H](Oc7ccc(C#N)c(Cl)c7)CC6)nn5)CC4)CC3)cc21 ; Cl]

A solution of 1-(1-isopropyl-6-(piperazine-1-carbonyl)-1H-indol-4-yl)dihydropyrimidine-2,4(1H,3H)-dione, (may be prepared as described in Example CB11; 37 mg, 0.096 mmol) in anhydrous DMF (0.7 mL) was added to 6-(4-(2-bromoethyl)piperidin-1-yl)-N-((1r,4r)-4-(3-chloro-4-cyanophenoxy)cyclohexyl)pyridazine-3-carboxamide (may be prepared as described in Description 112; 55 mg, 0.101 mmol), and triethylamine (26.8 L, 0.192 mmol) was added. The vessel was sealed and the mixture stirred at room temperature 22 h. The sealed vessel was stirred at 50° C. for 3 h. The mixture was allowed to cool to room temperature and purified by MDAP (high pH). The fractions were evaporated under a stream of nitrogen to give the product as a free base (36 mg, 0.042 mmol). A solution of the free base (23 mg, 0.027 mmol) in 1,4-Dioxane (0.50 mL) was treated with 4 M HCl in dioxane (10 μL, 0.040 mmol) and the mixture stirred at room temperature in a stoppered vessel for 2 h. The mixture was evaporated under a stream of nitrogen and the solid dried in a vacuum oven for 16 h to give the title compound as a white solid (16.9 mg, 0.019 mmol). LCMS (high pH A): Rt=1.20 min, MH⁺=849, 850, 851.

### Examples AR59-AR66

### General Procedure

A mixture of the appropriate bromide (0.051 mmol), the appropriate amine (0.056 mmol) and sodium bicarbonate (8.52 mg, 0.101 mmol) in DMF (0.8 mL) was heated at 60° C. for 3 h. The product mixture was cooled and subjected directly to purification by MDAP (high pH) to give the following compounds:

| | Name | | |
|---|---|---|---|
| Ex- | Analytical | | |
| ample | Mass; Yield | Bromide | Amine |
| AR59 | 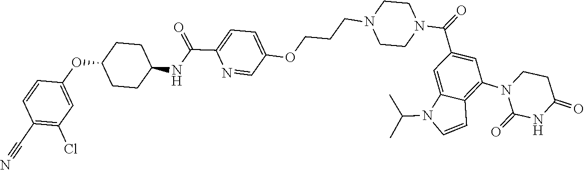 [SMILES: CC(C)n1ccc2c(N3CCC(=O)NC3=O)cc(C(=O)N3CCN(CCCOc4ccc(C(=O)N[C@H]5CC[C@H](Oc6ccc(C#N)c(Cl)c6)CC5)nc4)CC3)cc21] | D114 | ECB11 |
| | N-((1r,4r)-4-(3-Chloro-4-cyanophenoxy)cyclohexyl)-5-(3-(4-(4- | | |
| | (2,4-dioxotetrahydropyrimidin-1(2H)-yl)-1-isopropyl-1H-indole- | | |
| | 6-carbonyl)piperazin-1-yl)propoxy)picolinamide | | |
| | LCMS (formic A): Rt = 0.85 min, MH⁺ = 795 | | |
| | 15.4 mg, 38% | | |
| AR60 | 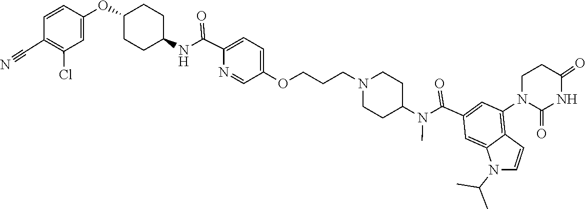 [SMILES: CC(C)n1ccc2c(N3CCC(=O)NC3=O)cc(C(=O)N(C)C3CCN(CCCOc4ccc(C(=O)N[C@H]5CC[C@H](Oc6ccc(C#N)c(Cl)c6)CC5)nc4)CC3)cc21] | D114 | ECB15 |
| | N-(1-(3-((6-(((1r,4r)-4-(3-Chloro-4- | | |
| | cyanophenoxy)cyclohexyl)carbamoyl)pyridin-3- | | |
| | yl)oxy)propyl)piperidin-4-yl)-4-(2,4-dioxotetrahydropyrimidin- | | |
| | 1(2H)-yl)-1-isopropyl-N-methyl-1H-indole-6-carboxamide | | |
| | LCMS (formic A): Rt = 0.84 min, MH⁺ = 823 | | |
| | 17.9 mg, 43% | | |
| AR61 | 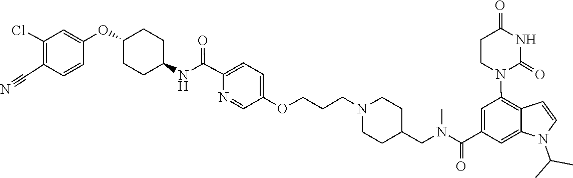 [SMILES: CC(C)n1ccc2c(N3CCC(=O)NC3=O)cc(C(=O)N(C)CC3CCN(CCCOc4ccc(C(=O)N[C@H]5CC[C@H](Oc6ccc(C#N)c(Cl)c6)CC5)nc4)CC3)cc21] | D114 | ECB16 |
| | N-((1-(3-((6-(((1r,4r)-4-(3-Chloro-4- | | |
| | cyanophenoxy)cyclohexyl)carbamoyl)pyridin-3- | | |
| | yl)oxy)propyl)piperidin-4-yl)methyl)-4-(2,4- | | |
| | dioxotetrahydropyrimidin-1(2H)-yl)-1-isopropyl-N-methyl-1H- | | |
| | indole-6-carboxamide | | |
| | LCMS (formic A): Rt = 0.86 min, MH⁺ = 837 | | |
| | 24.8 mg, 58% | | |
| AR62 | 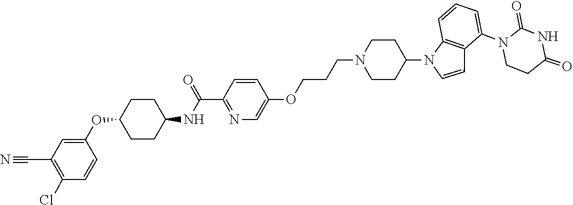 [SMILES: N#Cc1cc(O[C@H]2CC[C@H](NC(=O)c3ccc(OCCCN4CCC(n5ccc6c(N7CCC(=O)NC7=O)cccc65)CC4)cn3)CC2)ccc1Cl] | D114 | ECB1 |
| | N-((1r,4r)-4-(3-Chloro-4-cyanophenoxy)cyclohexyl)-5-(3-(4-(4- | | |
| | (2,4-dioxotetrahydropyrimidin-1(2H)-yl)-1H-indol-1- | | |
| | yl)piperidin-1-yl)propoxy)picolinamide | | |
| | LCMS (formic A): Rt = 0.83 min, MH⁺ = 724 | | |
| | 25.4 mg, 69% | | |
| AR63 | 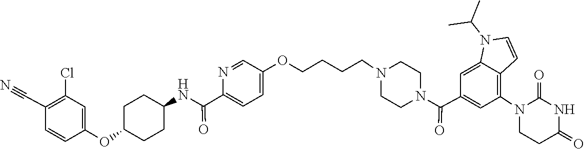 [SMILES: CC(C)n1ccc2c(N3CCC(=O)NC3=O)cc(C(=O)N3CCN(CCCCOc4ccc(C(=O)N[C@H]5CC[C@H](Oc6ccc(C#N)c(Cl)c6)CC5)nc4)CC3)cc21] | D115 | ECB11 |
| | N-((1r,4r)-4-(3-Chloro-4-cyanophenoxy)cyclohexyl)-5-(4-(4-(4- | | |
| | (2,4-dioxotetrahydropyrimidin-1(2H)-yl)-1-isopropyl-1H-indole- | | |
| | 6-carbonyl)piperazin-1-yl)butoxy)picolinamide | | |
| | LCMS (formic A): Rt = 0.82 min, MH⁺ = 809 | | |
| | 17.9 mg, 44% | | |
| AR64 | 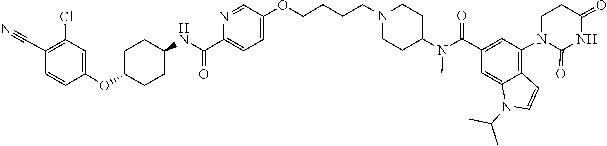 [SMILES: CC(C)n1ccc2c(N3CCC(=O)NC3=O)cc(C(=O)N(C)C3CCN(CCCCOc4ccc(C(=O)N[C@H]5CC[C@H](Oc6ccc(C#N)c(Cl)c6)CC5)nc4)CC3)cc21] | D115 | ECB15 |
| | N-(1-(4-((6-(((1r,4r)-4-(3-Chloro-4- | | |
| | cyanophenoxy)cyclohexyl)carbamoyl)pyridin-3- | | |
| | yl)oxy)butyl)piperidin-4-yl)-4-(2,4-dioxotetrahydropyrimidin- | | |
| | 1(2H)-yl)-1-isopropyl-N-methyl-1H-indole-6-carboxamide | | |
| | LCMS (formic A): Rt = 0.87 min, MH⁺ = 837 | | |
| | 19.4 mg, 46% | | |
| AR65 | 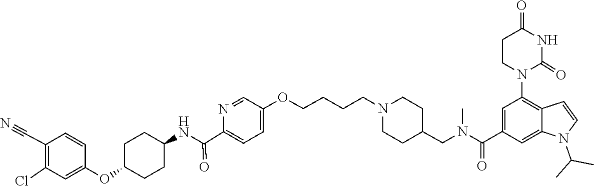 [SMILES: CC(C)n1ccc2c(N3CCC(=O)NC3=O)cc(C(=O)N(C)CC3CCN(CCCCOc4ccc(C(=O)N[C@H]5CC[C@H](Oc6ccc(C#N)c(Cl)c6)CC5)nc4)CC3)cc21] | D115 | ECB16 |
| | N-((1-(4-((6-(((1r,4r)-4-(3-Chloro-4- | | |
| | cyanophenoxy)cyclohexyl)carbamoyl)pyridin-3- | | |
| | yl)oxy)butyl)piperidin-4-yl)methyl)-4-(2,4- | | |
| | dioxotetrahydropyrimidin-1(2H)-yl)-1-isopropyl-N-methyl-1H- | | |
| | indole-6-carboxamide | | |
| | LCMS (formic A): Rt = 0.87 min, MH⁺ = 851 | | |
| | 24.5 mg, 57% | | |
| AR66 | 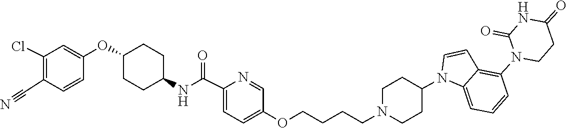 [SMILES: N#Cc1ccc(O[C@H]2CC[C@H](NC(=O)c3ccc(OCCCCN4CCC(n5ccc6c(N7CCC(=O)NC7=O)cccc65)CC4)cn3)CC2)cc1Cl] | D115 | ECB1 |
| | N-((1r,4r)-4-(3-chloro-4-cyanophenoxy)cyclohexyl)-5-(4-(4-(4- | | |
| | (2,4-dioxotetrahydropyrimidin-1(2H)-yl)-1H-indol-1- | | |
| | yl)piperidin-1-yl)butoxy)picolinamide | | |
| | LCMS (formic A): Rt = 0.84 min, MH⁺ = 738, 740 | | |
| | 12.8 mg, 34% | | |

### Example AR67

### N-((1r,3r)-3-(3-Chloro-4-cyanophenoxy)-2,2,4,4-tetramethylcyclobutyl)-2-(4-(4-(4-(2,4-dioxotetrahydropyrimidin-1(2H)-yl)-1H-indol-1-yl)piperidine-1-carbonyl)piperidin-1-yl)pyrimidine-5-carboxamide (EAR67)

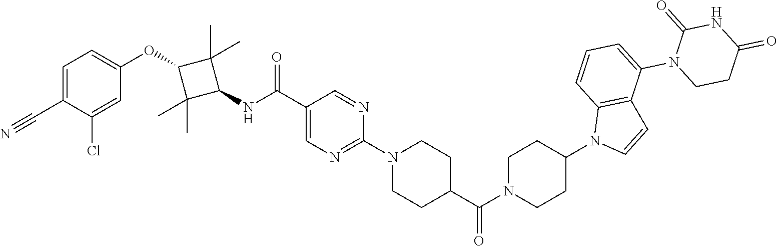 [SMILES: CC1(C)[C@H](NC(=O)c2cnc(N3CCC(C(=O)N4CCC(n5ccc6c(N7CCC(=O)NC7=O)cccc65)CC4)CC3)nc2)C(C)(C)[C@H]1Oc1ccc(C#N)c(Cl)c1]

A mixture of 1-(5-(((1r,3r)-3-(3-chloro-4-cyanophenoxy)-2,2,4,4-tetramethylcyclobutyl)carbamoyl)pyrimidin-2-yl)piperidine-4-carboxylic acid (may be prepared as described in Description 119; 25 mg, 0.049 mmol) and HATU (27 mg, 0.071 mmol) in anhydrous DMF (0.5 mL) was treated with DIPEA (0.017 mL, 0.096 mmol) and the solution stirred in a stoppered vessel at room temperature for 20 min. 1-(1-(Piperidin-4-yl)-1H-indol-4-yl)dihydropyrimidine-2,4(1H,3H)-dione (may be prepared as described in Example CB1; 15 mg, 0.048 mmol) was added and the mixture stirred in a stoppered vessel at room temperature for 3 h. The reaction mixture was diluted with EtOAc (2 mL) and washed sequentially with saturated NaHCO₃ (aq) (1 mL) and brine (1 mL). The organic layer was passed through a hydrophobic frit and the filtrate evaporated in vacuo. The resulting solid was dissolved in 2.01 mL DMSO and purified by MDAP (high pH) to give the title compound as a white solid (11.7 mg, 0.015 mmol). LCMS (formic A): Rt=1.26 min, MH⁺=806, 808.

### Example AR68

### N-((1r,4r)-4-(4-Cyano-3-(trifluoromethyl)phenoxy)cyclohexyl)-2-(4-(4-(4-(2,4-dioxotetrahydropyrimidin-1(2H)-yl)-1H-indol-1-yl)piperidine-1-carbonyl)piperidin-1-yl)pyrimidine-5-carboxamide (EAR68)

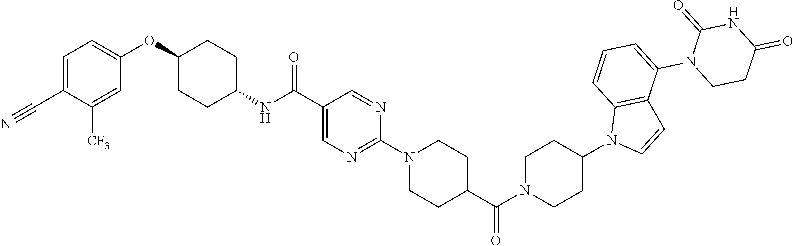 [SMILES: N#Cc1ccc(O[C@H]2CC[C@H](NC(=O)c3cnc(N4CCC(C(=O)N5CCC(n6ccc7c(N8CCC(=O)NC8=O)cccc76)CC5)CC4)nc3)CC2)cc1C(F)(F)F]

A mixture of 1-(5-(((1r,4r)-4-(4-cyano-3-(trifluoromethyl)phenoxy)cyclohexyl)carbamoyl)pyrimidin-2-yl)piperidine-4-carboxylic acid (may be prepared as described in Description 118; 33 mg, 0.064 mmol) and HATU (36 mg, 0.095 mmol) in anhydrous DMF (0.5 mL) was treated with DIPEA (0.022 mL, 0.128 mmol), and the solution was stirred in a stoppered vessel at room temperature for 20 min. 1-(1-(Piperidin-4-yl)-1H-indol-4-yl)dihydropyrimidine-2,4(1H,3H)-dione (may be prepared as described in Example CB1; 20 mg, 0.064 mmol) was added, and the mixture was stirred in a stoppered vessel at room temperature for 3 h. The reaction mixture was diluted with EtOAc (2 mL) and washed sequentially with saturated NaHCO₃ (aq) (1 mL) and brine (1 mL). The organic layer was passed through a hydrophobic frit and the filtrate evaporated in vacuo. The resulting solid was dissolved in 3 mL MeOH and purified on a Torus DIOL (150×30 mm, 5 μm) column eluting with 25-55% 0.5% v/v Isopropylamine in MeOH in super-critical CO₂ to give the title compound. LCMS (formic A): Rt=1.15 min, MH⁺=812.

### Example AR69

### N-((1r,4r)-4-(3-Chloro-4-cyanophenoxy)cyclohexyl)-2-(4-(4-(4-(2,4-dioxotetrahydropyrimidin-1(2H)-yl)-1H-indol-1-yl)piperidine-1-carbonyl)piperidin-1-yl)pyrimidine-5-carboxamide (EAR69)

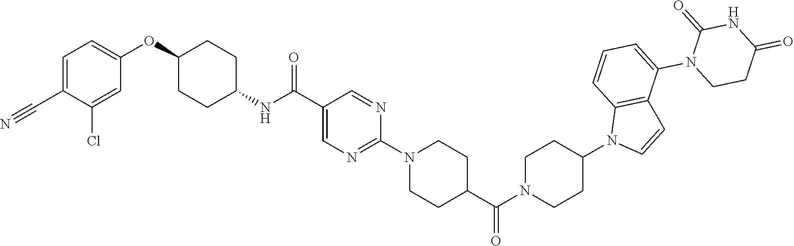 [SMILES: N#Cc1ccc(O[C@H]2CC[C@H](NC(=O)c3cnc(N4CCC(C(=O)N5CCC(n6ccc7c(N8CCC(=O)NC8=O)cccc76)CC5)CC4)nc3)CC2)cc1Cl]

A mixture of 1-(5-(((1r,4r)-4-(3-chloro-4-cyanophenoxy)cyclohexyl)carbamoyl)pyrimidin-2-yl)piperidine-4-carboxylic acid (may be prepared as described in Description 117; 37 mg, 0.076 mmol) and HATU (42.0 mg, 0.110 mmol) in anhydrous DMF (0.5 mL) was treated with DIPEA (0.026 mL, 0.147 mmol), and the solution was stirred in a stoppered vessel at room temperature for 20 min. 1-(1-(Piperidin-4-yl)-1H-indol-4-yl)dihydropyrimidine-2,4(1H,3H)-dione (may be prepared as described in Example CB1; 23 mg, 0.074 mmol) was added, and the mixture was stirred in a stoppered vessel at room temperature for 3 h. The reaction mixture was diluted with EtOAc (2 mL) and washed sequentially with saturated NaHCO₃ (aq) (1 mL) and brine (1 mL). The organic layer was passed through a hydrophobic frit and the filtrate evaporated in vacuo. The resulting solid was dissolved in 1.9 mL DMSO and purified on an XBridge Prep Shield C18 (150×30 mm, 5 μm) column eluting with 35-100% 10 mM NH₄HCO₃ in water adjusted to pH 10 with aqueous NH₃ in MeCN to yield the title compound as a white solid (24 mg, 0.031 mmol, 42% yield). LCMS (formic A): Rt=1.12 min, MH⁺=778, 780.

### Example AR70

### N-((1r,4r)-4-(3-Chloro-4-cyanophenoxy)cyclohexyl)-6-(4-(4-(2,4-dioxotetrahydropyrimidin-1(2H)-yl)-1H-indol-1-yl)piperidin-1-yl)pyridazine-3-carboxamide (EAR70)

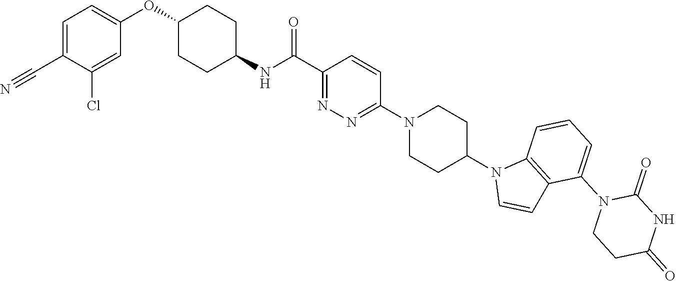 [SMILES: N#Cc1ccc(O[C@H]2CC[C@H](NC(=O)c3ccc(N4CCC(n5ccc6c(N7CCC(=O)NC7=O)cccc65)CC4)nn3)CC2)cc1Cl]

6-Chloro-N-((1r,4r)-4-(3-chloro-4-cyanophenoxy)cyclohexyl)pyridazine-3-carboxamide (may be prepared as described in Description 90; 20 mg, 0.051 mmol) (15.97 mg, 0.051 mmol) and DIPEA (0.018 mL, 0.102 mmol) were combined in DMSO (0.4 mL) in a microwave vial, and the mixture was heated at 70° C. for 1 h, then allowed to cool to room temperature and aged over the weekend to a dense suspension. This was diluted with MeOH (0.5 mL) and filtered. The filtrate was evaporated in vacuo and the residue dissolved in DMSO (1 mL), then purified by MDAP (formic) to give the title compound as a colourless solid (9 mg, 0.013 mmol, 26% yield). LCMS (formic A): Rt=1.14 min, MH⁺=667, 669.

### Example AR71

### N-((1r,4r)-4-(3-Chloro-4-cyanophenoxy)cyclohexyl)-6-(4-((4-(4-(2,4-dioxotetrahydropyrimidin-1(2H)-yl)-1H-pyrrolo[2,3-c]pyridin-1-yl)piperidin-1-yl)methyl)-4-fluoropiperidin-1-yl)pyridazine-3-carboxamide (EAR71)

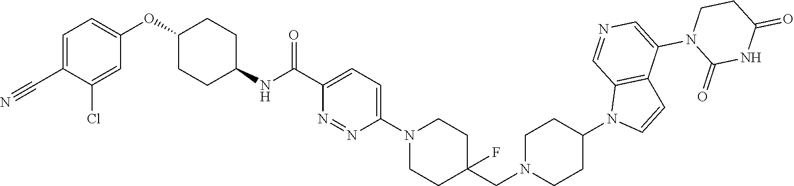 [SMILES: N#Cc1ccc(O[C@H]2CC[C@H](NC(=O)c3ccc(N4CCC(F)(CN5CCC(n6ccc7c(N8CCC(=O)NC8=O)cncc76)CC5)CC4)nn3)CC2)cc1Cl]

A mixture of 1-(1-(1-((4-fluoropiperidin-4-yl)methyl)piperidin-4-yl)-1H-pyrrolo[2,3-c]pyridin-4-yl)dihydropyrimidine-2,4(1H,3H)-dione hydrochloride (may be prepared as described in Example CBL3; 70 mg, 0.151 mmol), 6-chloro-N-((1r,4r)-4-(3-chloro-4-cyanophenoxy)cyclohexyl)pyridazine-3-carboxamide (may be prepared as described in Description 90; 64 mg, 0.164 mmol) and DIPEA (64 mg, 0.086 mL) in N-methyl-2-pyrrolidone (2 mL) was heated at 100° C. for 16 h. The reaction mixture was cooled to room temperature. The mixture was partitioned between EtOAc (10 mL) and water (10 mL). The organic phase was separated. the aqueous phase was extracted with EtOAc (3×5 mL). The combined organics were washed with brine (10 mL), dried and evaporated. The residue was chromatographed [50-100% EtOAc/cyclohexane, 0-30% ethanol/EtOAc] to give the title compound as an off white solid (24 mg, 0.031 mmol, 20% yield). LCMS (high pH A): Rt=1.87 min, MH⁺=783, 785.

### Example AR72

### N-((1r,4r)-4-(3-Chloro-4-cyanophenoxy)cyclohexyl)-6-(3-((4-(4-(2,4-dioxotetrahydropyrimidin-1(2H)-yl)-1H-indol-1-yl)piperidin-1-yl)methyl) azetidin-1-yl)pyridazine-3-carboxamide hydrochloride (EAR72)

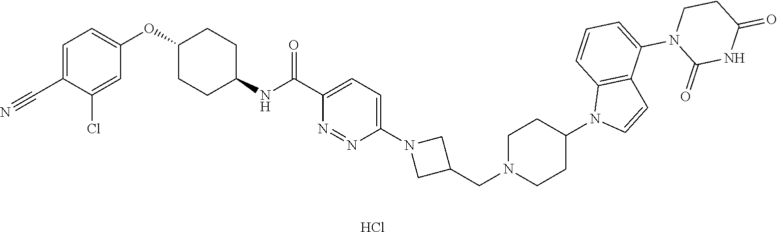 [SMILES: N#Cc1ccc(O[C@H]2CC[C@H](NC(=O)c3ccc(N4CC(CN5CCC(n6ccc7c(N8CCC(=O)NC8=O)cccc76)CC5)C4)nn3)CC2)cc1Cl ; Cl]

To 1-(1-(1-(azetidin-3-ylmethyl)piperidin-4-yl)-1H-indol-4-yl)dihydropyrimidine-2,4(1H,3H)-dione (may be prepared as described in Example CBL1; 57 mg, 0.15 mmol) was added 6-chloro-N-((1r,4r)-4-(3-chloro-4-cyanophenoxy)cyclohexyl)pyridazine-3-carboxamide (may be prepared as described in Description 90; 0.059 g, 0.150 mmol) and DIPEA (0.079 mL, 0.450 mmol) dissolved in DMSO (0.5 mL). The reaction was left to stir at 60° C. for 18 h. The reaction purified by MDAP (formic). The solvent was dried under a stream of nitrogen to give the desired product, which was dissolved in 1 mL of 1:1 DCM:MeOH and then passed through pre-conditioned Agilent StratoSpheres PL-HCO3 MP SPE cartridge (200 mg, MeOH 1 mL). The cartridge was then washed with additional MeOH 2 mL and the solvent was dried under a stream of nitrogen. To this material was added 250 μL of 4 M HCl in 1,4-dioxane, and the reaction was stirred at room temperature for 2 h. The solvent was dried under a stream of nitrogen the products as HCL salts. The material was dissolved in 1:1 MeOH:DMSO 1 mL and purified by MDAP (formic). The product was then passed through a pre-conditioned Agilent StratoSpheres PL-HCO3 MP SPE cartridge (200 mg, MeOH 1 mL) then washed with additional MeOH 3 mL. The solvent was dried under a stream of nitrogen to give the title compound (16.2 mg, 0.021 mmol, 11.18%). LCMS (high pH A): Rt=0.78 min, MH⁺=736, 738.

### Example AR73

### rac-N-((1r,4r)-4-(3-Chloro-4-cyanophenoxy)cyclohexyl)-6-(3-((4-(4-(2,4-dioxotetrahydropyrimidin-1(2H)-yl)-1H-indol-1-yl)piperidin-1-yl)methyl)piperidin-1-yl)pyridazine-3-carboxamide (EAR73)

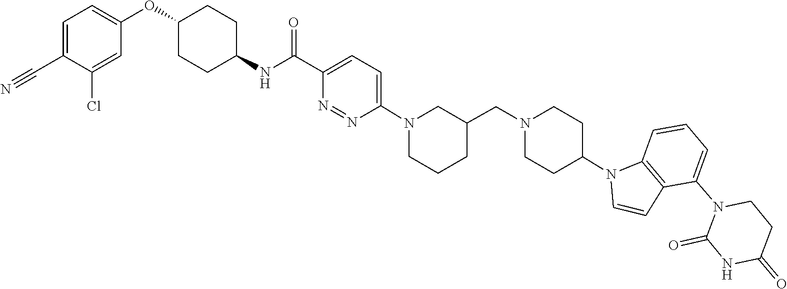 [SMILES: N#Cc1ccc(O[C@H]2CC[C@H](NC(=O)c3ccc(N4CCCC(CN5CCC(n6ccc7c(N8CCC(=O)NC8=O)cccc76)CC5)C4)nn3)CC2)cc1Cl]

1-(1-(Piperidin-4-yl)-1H-indol-4-yl)dihydropyrimidine-2,4(1H,3H)-dione (may be prepared as described in Example CB1; 80 mg, 0.256 mmol) and N-((1r,4r)-4-(3-chloro-4-cyanophenoxy)cyclohexyl)-6-(3-formylpiperidin-1-yl)pyridazine-3-carboxamide (may be prepared as described in Description 102; 90 mg, 0.192 mmol) were was suspended in DCM (20 mL) at room temperature, then sodium triacetoxyborohydride (109 mg, 0.512 mmol) was added, and the mixture was stirred for 1 h. Sodium triacetoxyborohydride (109 mg, 0.512 mmol) was added, and the mixture was stirred for 2 h. Sodium bicarbonate solution (20 mL) was added, and the mixture was stirred for 20 min, then the phases were separated and the organic layer dried and evaporated in vacuo to give a pale yellow gum. The crude material was dissolved in DCM and loaded onto a 24 g silica column, then eluted with 0-20% 2 M MeOH ammonia/DCM and product-containing fractions were evaporated in vacuo to give the title compound (110 mg, 0.144 mmol, 56% yield) as a coloulress solid. LCMS (high pH A): Rt=1.32 min, MH⁺=764, 766.

### Example AR74

### N-((1r,4S)-4-(3-Chloro-4-cyanophenoxy)cyclohexyl)-6-((S)-3-((4-(4-(2,4-dioxotetrahydropyrimidin-1(2H)-yl)-1H-indol-1-yl)piperidin-1-yl)methyl)piperidin-1-yl)pyridazine-3-carboxamide hydrochloride (EAR74)

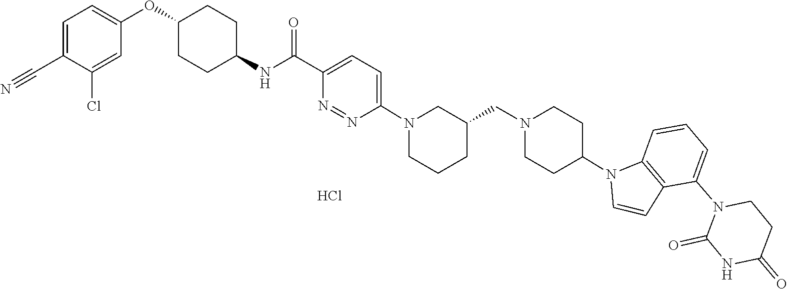 [SMILES: N#Cc1ccc(O[C@H]2CC[C@H](NC(=O)c3ccc(N4CCC[C@@H](CN5CCC(n6ccc7c(N8CCC(=O)NC8=O)cccc76)CC5)C4)nn3)CC2)cc1Cl ; Cl]

To (R)-1-(1-(1-(piperidin-3-ylmethyl)piperidin-4-yl)-1H-indol-4-yl)dihydropyrimidine-2,4(1H,3H)-dione (may be prepared as described in Example CBL2; 61 mg, 0.15 mmol) was added 6-chloro-N-((1r,4r)-4-(3-chloro-4-cyanophenoxy)cyclohexyl)pyridazine-3-carboxamide (may be prepared as described in Description 90; 0.059 g, 0.150 mmol) and DIPEA (0.079 mL, 0.450 mmol) dissolved in DMSO (0.5 mL). The reaction was left to stir 60° C. for 18 h. The reaction purified by MDAP (formic). The solvent was dried under a stream of nitrogen to give the desired product, which was dissolved in 1 mL of 1:1 DCM:MeOH and then passed through pre-conditioned Agilent StratoSpheres PL-HCO3 MP SPE cartridge (200 mg, MeOH 1 mL). The cartridge was then washed with additional MeOH (2 mL) and the solvent was dried under a stream of nitrogen. To this material was added 250 μL of 4 M HCl in 1,4-dioxane, and the reaction was stirred at room temperature for 2 h. The solvent was dried under a stream of nitrogen the products as HCl salts. The material was dissolved in 1:1 MeOH:DMSO (1 mL) and purified by MDAP (formic). The product was then passed through a pre-conditioned Agilent StratoSpheres PL-HCO3 MP SPE cartridge (200 mg, MeOH 1 mL) then washed with additional MeOH 3 mL. The solvent was dried under a stream of nitrogen to give the title compound (12.5 mg, 0.016 mmol, 8.33%). LCMS (high pH A): Rt=0.82 min, MH⁺=764, 766.

### Example AR75

### N-((1r,3r)-3-(4-Cyano-3-(trifluoromethyl)phenoxy)-2,2,4,4-tetramethylcyclobutyl)-4-(6-(4-(4-(2,4-dioxotetrahydropyrimidin-1(2H)-yl)-1H-indol-1-yl)piperidin-1-yl)hexyl)benzamide (EAR75)

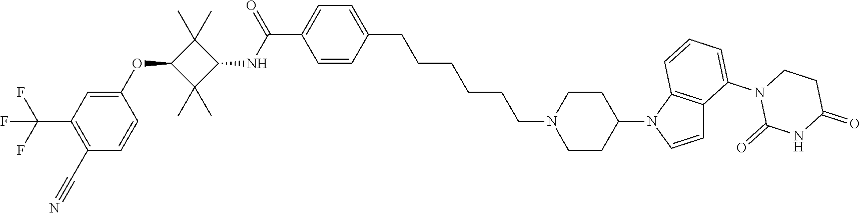 [SMILES: CC1(C)[C@H](NC(=O)c2ccc(CCCCCCN3CCC(n4ccc5c(N6CCC(=O)NC6=O)cccc54)CC3)cc2)C(C)(C)[C@H]1Oc1ccc(C#N)c(C(F)(F)F)c1]

A mixture of 6-(4-(((1r,3r)-3-(4-cyano-3-(trifluoromethyl)phenoxy)-2,2,4,4-tetramethylcyclobutyl)carbamoyl)phenyl)hexyl 4-methylbenzenesulfonate (may be prepared as described in Description 111; 100 mg, 0.149 mmol), 1-(1-(piperidin-4-yl)-1H-indol-4-yl)dihydropyrimidine-2,4(1H,3H)-dione (may be prepared as described in Example CB1; 51.2 mg, 0.164 mmol), N-methylmorpholine (0.049 mL, 0.447 mmol) and sodium iodide (2.235 mg, 0.015 mmol) in DMF (2 mL) was stirred at 80° C. for 2 h. The product was subjected directly to purification by MDAP (formic) to give the title compound as a white solid (42 mg, 0.052 mmol, 35% yield). LCMS (formic A): Rt=1.01 min, MH⁺=811.

### Example AR76

### N-(2-(2-(4-(6-(((1r,4r)-4-(3-Chloro-4-cyanophenoxy)cyclohexyl)carbamoyl)pyridazin-3-yl)piperazin-1-yl)ethoxy)ethyl)-4-(2,4-dioxotetrahydropyrimidin-1(2H)-yl)-1-isopropyl-1H-indole-6-carboxamide (EAR76)

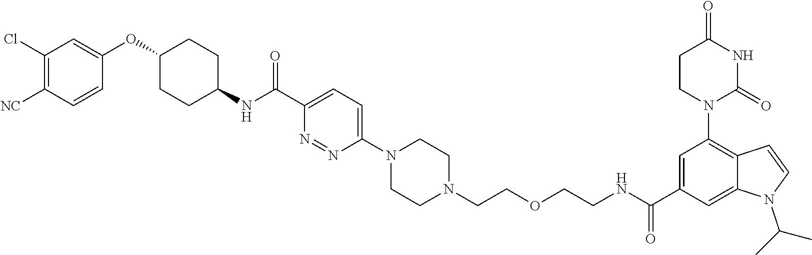 [SMILES: CC(C)n1ccc2c(N3CCC(=O)NC3=O)cc(C(=O)NCCOCCN3CCN(c4ccc(C(=O)N[C@H]5CC[C@H](Oc6ccc(C#N)c(Cl)c6)CC5)nn4)CC3)cc21]

A mixture of 4-(2,4-dioxotetrahydropyrimidin-1(2H)-yl)-1-isopropyl-1H-indole-6-carboxylic acid (may be prepared as described in Description 35; 16.21 mg, 0.051 mmol), 6-(4-(2-(2-aminoethoxy)ethyl)piperazin-1-yl)-N-((1r,4r)-4-(3-chloro-4-cyanophenoxy)cyclohexyl)pyridazine-3-carboxamide trifluoroacetate (may be prepared as described in Description 123; 30 mg, 0.047 mmol) and triethylamine (0.033 mL, 0.234 mmol) in DMF (0.8 mL) was treated with HATU (24.87 mg, 0.065 mmol) and stirred at ambient temperature for 2 h. The purified directly by MDAP (high pH) to give the title compound (8.1 mg, 9.81 μmol, 21% yield). LCMS (formic A): Rt=0.83 min, MH⁺=825, 827.

### Example AR77

### N-((1r,4r)-4-(3-Chloro-4-cyanophenoxy)cyclohexyl)-6-(4-(4-(2,4-dioxotetrahydropyrimidin-1(2H)-yl)-1-isopropyl-1H-indole-6-carbonyl)piperazin-1-yl)pyridazine-3-carboxamide (EAR77)

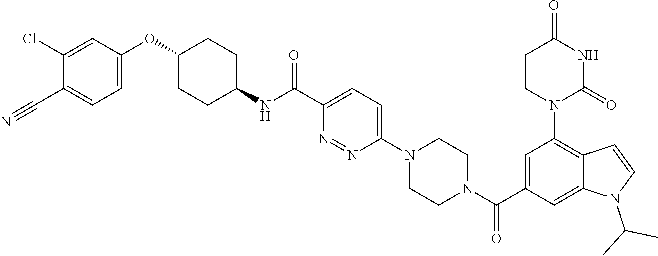 [SMILES: CC(C)n1ccc2c(N3CCC(=O)NC3=O)cc(C(=O)N3CCN(c4ccc(C(=O)N[C@H]5CC[C@H](Oc6ccc(C#N)c(Cl)c6)CC5)nn4)CC3)cc21]

A mixture of 6-chloro-N-((1r,4r)-4-(3-chloro-4-cyanophenoxy)cyclohexyl)pyridazine-3-carboxamide (may be prepared as described in Description 90; 10 mg, 0.026 mmol), 1-(1-isopropyl-6-(piperazine-1-carbonyl)-1H-indol-4-yl)dihydropyrimidine-2,4(1H,3H)-dione (may be prepared as described in Example CB11; 9.80 mg, 0.026 mmol), and N-methylmorpholine (8.43 μl, 0.077 mmol) in DMSO (85 μl) was stirred at 60° C. for 4 h. Reaction was left to stir at 60° C. overnight. Reaction was stirred at 60° C. for an additional 48 h. The reaction mixture was diluted in DMSO (0.5 mL) and purified by MDAP (high pH). The solvent was dried under a stream of nitrogen to give the title compound (5.4 mg, 7.31 μmol, 29% yield). LCMS (high pH A): Rt=1.13 min, MH⁺=739.

### Example AR78

### N-((1r,3r)-3-(3-Chloro-4-cyanophenoxy)-2,2,4,4-tetramethylcyclobutyl)-6-(4-((4-(4-(2,4-dioxotetrahydropyrimidin-1(2H)-yl)-1-isopropyl-1H-indole-6-carbonyl)piperazin-1-yl)methyl)piperidin-1-yl)pyridazine-3-carboxamide (EAR78)

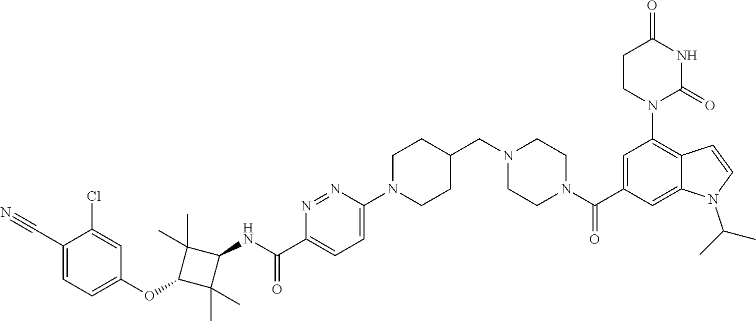 [SMILES: CC(C)n1ccc2c(N3CCC(=O)NC3=O)cc(C(=O)N3CCN(CC4CCN(c5ccc(C(=O)N[C@H]6C(C)(C)[C@H](Oc7ccc(C#N)c(Cl)c7)C6(C)C)nn5)CC4)CC3)cc21]

N-((1r,3r)-3-(3-chloro-4-cyanophenoxy)-2,2,4,4-tetramethylcyclobutyl)-6-(4-formylpiperidin-1-yl)pyridazine-3-carboxamide (may be prepared as described in Description 104; 52 mg, 0.105 mmol), 1-(1-isopropyl-6-(piperazine-1-carbonyl)-1H-indol-4-yl)dihydropyrimidine-2,4(1H,3H)-dione (may be prepared as described in Example CB11; 40 mg, 0.104 mmol) and sodium triacetoxyborohydride (66.7 mg, 0.315 mmol) were dissolved in DCM (5 mL) and the mixture stirred at room temperature for 2 h, then washed with saturated sodium bicarbonate solution and the organic layer dried and evaporated in vacuo to give a colourless gum. This was dissolved in DCM and loaded onto a 12 g silica column, then eluted with 0-100% (25% ethanol/EtOAc 1% NH4OH) and product-containing fractions evaporated in vacuo to give the title compound as a colourless solid (67 mg, 0.078 mmol, 74% yield). LCMS (high pH A): Rt=1.37 min, MH⁺=863, 865.

### Example AR79

### N-((1r,4r)-4-(3-Chloro-4-cyanophenoxy)cyclohexyl)-6-(4-((4-(4-(2,4-dioxotetrahydropyrimidin-1(2H)-yl)-1-isopropyl-1H-indole-6-carbonyl)piperazin-1-yl)methyl)piperidin-1-yl)pyridazine-3-carboxamide (EAR79)

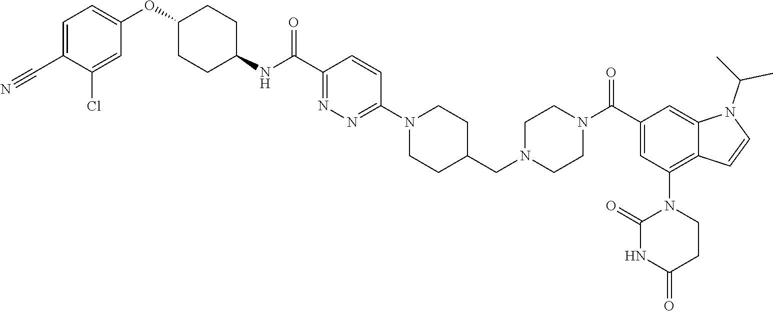 [SMILES: CC(C)n1ccc2c(N3CCC(=O)NC3=O)cc(C(=O)N3CCN(CC4CCN(c5ccc(C(=O)N[C@H]6CC[C@H](Oc7ccc(C#N)c(Cl)c7)CC6)nn5)CC4)CC3)cc21]

To a stirred solution of N-((1r,4r)-4-(3-chloro-4-cyanophenoxy)cyclohexyl)-6-(4-formylpiperidin-1-yl)pyridazine-3-carboxamide (may be prepared as described in Description 95; 66 mg, 0.141 mmol) and 1-(1-isopropyl-6-(piperazine-1-carbonyl)-1H-indol-4-yl)dihydropyrimidine-2,4(1H,3H)-dione (may be prepared as described in Example CB11; 54.1 mg, 0.141 mmol) in DCM (1500 μl) and DMF (500 μl) was added sodium triacetoxyborohydride (90 mg, 0.423 mmol), and the reaction mixture was stirred at room temperature for 30 minutes. The reaction mixture was diluted with saturated aqueous sodium hydrogen carbonate (10 mL) and extracted with DCM (3×10 mL). The organic layers were combined, passed through a hydrophobic frit and the solvent removed in vacuo. The residue was purified by MDAP (high pH) to give the title compound as a white solid (70 mg, 0.084 mmol, 59% yield). LCMS (high pH A): Rt=1.20 min, MH⁺=835, 837.

### Example AR80

### N-((1r,4r)-4-(3-Chloro-4-cyanophenoxy)cyclohexyl)-6-(4-((4-(4-(2,4-dioxotetrahydropyrimidin-1(2H)-yl)-1-isopropyl-1H-indole-6-carbonyl)piperazin-1-yl)methyl)piperidin-1-yl)nicotinamide (EAR80)

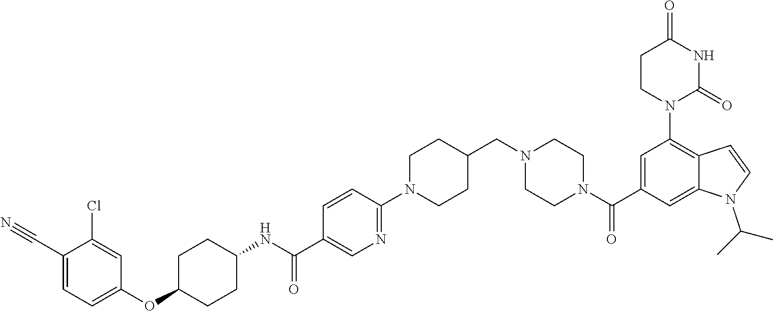 [SMILES: CC(C)n1ccc2c(N3CCC(=O)NC3=O)cc(C(=O)N3CCN(CC4CCN(c5ccc(C(=O)N[C@H]6CC[C@H](Oc7ccc(C#N)c(Cl)c7)CC6)cn5)CC4)CC3)cc21]

2-Picoline borane (20 mg, 0.187 mmol) was added to a mixture of N-((1r,4r)-4-(3-chloro-4-cyanophenoxy)cyclohexyl)-6-(4-formylpiperidin-1-yl)nicotinamide (may be prepared as described in Description 94; 30 mg, 0.064 mmol) and 1-(1-isopropyl-6-(piperazine-1-carbonyl)-1H-indol-4-yl)dihydropyrimidine-2,4(1H,3H)-dione, (may be prepared as described in Example CB11; 24.63 mg, 0.064 mmol) in DCM (1 mL) and the mixture was stirred at room temperature for 1 h. The solution was loaded onto a 21 g silica column and eluted with 0-100% (25% ethanol/EtOAc 1% NH4OH). Product-containing fractions were evaporated in vacuo to give the title compound as a colourless solid (23 mg, 0.028 mmol, 43% yield). LCMS (formic A): Rt=0.78 min, MH⁺=834.

### Example AR81

### N-((1r,4r)-4-(3-Chloro-4-cyanophenoxy)cyclohexyl)-4-((9-(4-(2,4-dioxotetrahydropyrimidin-1(2H)-yl)-1-isopropyl-1H-indole-6-carbonyl)-2,9-diazaspiro[5.5]undecan-2-yl)methyl)benzamide (EAR81)

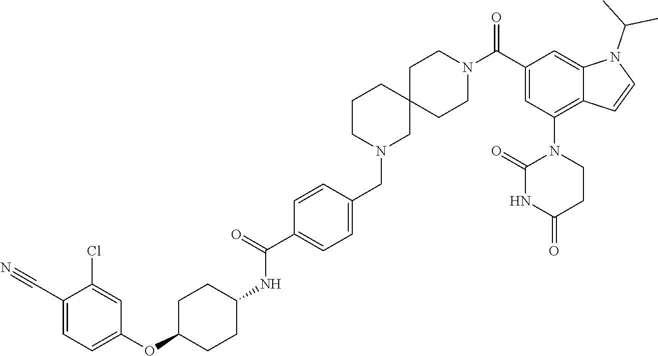 [SMILES: CC(C)n1ccc2c(N3CCC(=O)NC3=O)cc(C(=O)N3CCC4(CCCN(Cc5ccc(C(=O)N[C@H]6CC[C@H](Oc7ccc(C#N)c(Cl)c7)CC6)cc5)C4)CC3)cc21]

1-(1-Isopropyl-6-(2,9-diazaspiro[5.5]undecane-9-carbonyl)-1H-indol-4-yl)dihydropyrimidine-2,4(1H,3H)-dione (may be prepared as described in Example CB12; 56.6 mg, 0.125 mmol) and N-((1r,4r)-4-(3-chloro-4-cyanophenoxy)cyclohexyl)-4-formylbenzamide (may be prepared as described in Description 105; 48 mg, 0.125 mmol) was dissolved in DCM (3 mL) and stirred at room temperature for 10 min. Triethylamine (0.035 mL, 0.251 mmol) and sodium triacetoxyborohydride (80 mg, 0.376 mmol) were then added to the reaction mixture and stirring was continued at room temperature for 3 h. The reaction mixture was quenched with aqueous sodium bicarbonate solution (10 mL) and extracted with EtOAc (3×10 mL). The combined organic extracts were dried and evaporated. The residue was chromatographed [0-15% ethanol/EtOAc]. The combined fractions were evaporated and the residue was purified by high pH MDAP to give the title compound as an off white solid (39 mg, 0.048 mmol, 38% yield). LCMS (high pH A): Rt=1.33 min, MH⁺=818.

### Example AR82

### N-((1r,4r)-4-(3-Chloro-4-cyanophenoxy)cyclohexyl)-4-(3-(9-(4-(2,4-dioxotetrahydropyrimidin-1(2H)-yl)-1-isopropyl-1H-indole-6-carbonyl)-2,9-diazaspiro[5.5]undecan-2-yl)propyl)benzamide (EAR82)

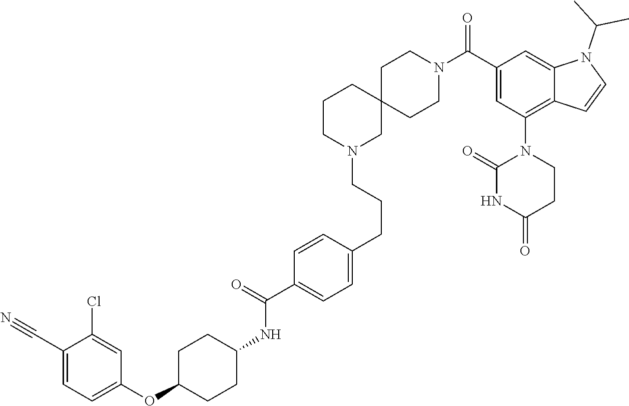 [SMILES: CC(C)n1ccc2c(N3CCC(=O)NC3=O)cc(C(=O)N3CCC4(CCCN(CCCc5ccc(C(=O)N[C@H]6CC[C@H](Oc7ccc(C#N)c(Cl)c7)CC6)cc5)C4)CC3)cc21]

1-(1-Isopropyl-6-(2,9-diazaspiro[5.5]undecane-9-carbonyl)-1H-indol-4-yl)dihydropyrimidine-2,4(1H,3H)-dione (may be prepared as described in Example CB12; 47.3 mg, 0.105 mmol) and N-((1r,4r)-4-(3-chloro-4-cyanophenoxy)cyclohexyl)-4-(3-oxopropyl)benzamide (may be prepared as described in Description 106; 43 mg, 0.105 mmol) was dissolved in DCM (3 mL) and stirred at room temperature for 10 min. triethylamine (0.029 mL, 0.209 mmol) and sodium triacetoxyborohydride (66.5 mg, 0.314 mmol) were then added to the reaction mixture and stirring was continued at room temperature for 4 h. The reaction mixture was quenched with aqueous sodium bicarbonate solution (10 mL) and extracted with DCM (3×10 mL). The combined organic extracts were dried and evaporated. The residue was chromatographed [0-10% ethanol/DCM] then purified by MDAP (high pH) to give the title compound as an off white solid (17 mg, 0.020 mmol, 19% yield). LCMS (high pH A): Rt=1.36 min, MH⁺=846.

### Example AR83

### N-((1r,4r)-4-(3-Chloro-4-cyanophenoxy)cyclohexyl)-4-(5-(9-(4-(2,4-dioxotetrahydropyrimidin-1(2H)-yl)-1-isopropyl-1H-indole-6-carbonyl)-2,9-diazaspiro[5.5]undecan-2-yl)pentyl)benzamide (EAR83)

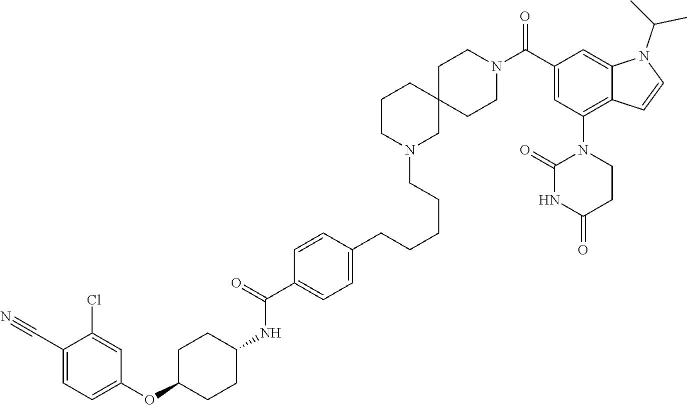 [SMILES: CC(C)n1ccc2c(N3CCC(=O)NC3=O)cc(C(=O)N3CCC4(CCCN(CCCCCc5ccc(C(=O)N[C@H]6CC[C@H](Oc7ccc(C#N)c(Cl)c7)CC6)cc5)C4)CC3)cc21]

1-(1-Isopropyl-6-(2,9-diazaspiro[5.5]undecane-9-carbonyl)-1H-indol-4-yl)dihydropyrimidine-2,4(1H,3H)-dione (may be prepared as described in Example CB12; 15.43 mg, 0.034 mmol) and N-((1r,4r)-4-(3-chloro-4-cyanophenoxy)cyclohexyl)-4-(5-oxopentyl)benzamide (may be prepared as described in Description 107; 15 mg, 0.034 mmol) was dissolved in DCM (3 mL) and stirred at room temperature for 10 min. triethylamine (0.014 mL, 0.103 mmol) and sodium triacetoxyborohydride (15 mg, 0.071 mmol) were then added to the reaction mixture and stirring was continued at room temperature for 4 h. The reaction mixture was quenched with aqueous sodium bicarbonate solution (10 mL) and extracted with DCM (3×10 mL). The combined organic extracts were dried and evaporated. The residue was chromatographed [0-10% ethanol/DCM] to give the title compound as an off white solid (20 mg, 0.023 mmol, 67% yield). LCMS (high pH A): Rt=1.43 min, MH⁺=874.

### Example AR84

### N-((1r,4r)-4-(3-Chloro-4-cyanophenoxy)cyclohexyl)-4-((9-(4-(2,4-dioxotetrahydropyrimidin-1(2H)-yl)-1-isopropyl-1H-indole-6-carbonyl)-3,9-diazaspiro[5.5]undecan-3-yl)methyl)benzamide (EAR84)

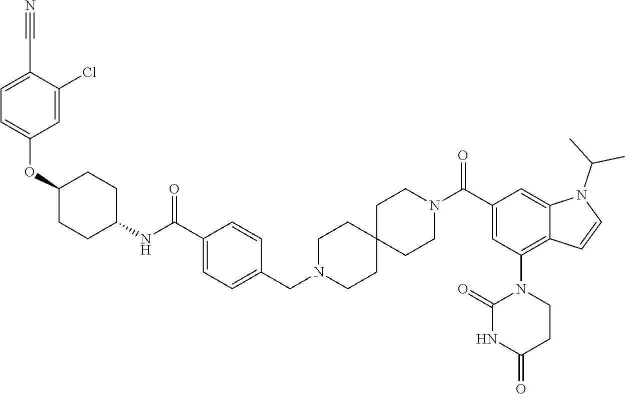 [SMILES: CC(C)n1ccc2c(N3CCC(=O)NC3=O)cc(C(=O)N3CCC4(CCN(Cc5ccc(C(=O)N[C@H]6CC[C@H](Oc7ccc(C#N)c(Cl)c7)CC6)cc5)CC4)CC3)cc21]

1-(1-Isopropyl-6-(3,9-diazaspiro[5.5]undecane-3-carbonyl)-1H-indol-4-yl)dihydropyrimidine-2,4(1H,3H)-dione (may be prepared as described in Example CB13; 41 mg, 0.091 mmol) and N-((1r,4r)-4-(3-chloro-4-cyanophenoxy)cyclohexyl)-4-formylbenzamide (may be prepared as described in Description 105; 34.8 mg, 0.091 mmol) was dissolved in DCM (3 mL) and stirred at room temperature for 10 min. Triethylamine (0.025 mL, 0.182 mmol) and sodium triacetoxyborohydride (57.7 mg, 0.272 mmol) were then added to the reaction mixture and stirring was continued at room temperature for 2 h. Another portion of sodium triacetoxyborohydride (57.7 mg, 0.272 mmol) was added and stirring continued for a further hour. This step was repeated once more. The reaction mixture was quenched with aqueous sodium bicarbonate solution (10 mL) and extracted with DCM (3×10 mL). The combined organic extracts were dried and evaporated. The residue was chromatographed [0-20% ethanol/DCM]. The combined fractions of were evaporated and the residue was purified by MDAP (high pH) to give the title compound as an off white solid (25 mg, 0.031 mmol, 34% yield). LCMS (high pH A): Rt=1.20 min, MH⁺=818.

### Example AR85

### N-((1r,4r)-4-(3-Chloro-4-cyanophenoxy)cyclohexyl)-4-(3-(9-(4-(2,4-dioxotetrahydropyrimidin-1(2H)-yl)-1-isopropyl-1H-indole-6-carbonyl)-3,9-diazaspiro[5.5]undecan-3-yl)propyl)benzamide (EAR85)

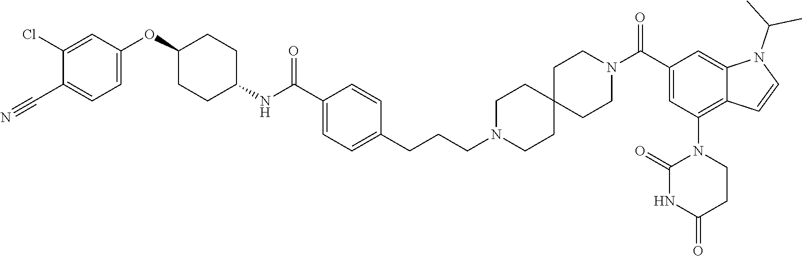 [SMILES: CC(C)n1ccc2c(N3CCC(=O)NC3=O)cc(C(=O)N3CCC4(CCN(CCCc5ccc(C(=O)N[C@H]6CC[C@H](Oc7ccc(C#N)c(Cl)c7)CC6)cc5)CC4)CC3)cc21]

1-(1-Isopropyl-6-(3,9-diazaspiro[5.5]undecane-3-carbonyl)-1H-indol-4-yl)dihydropyrimidine-2,4(1H,3H)-dione (may be prepared as described in Example CB13; 41 mg, 0.091 mmol) and N-((1r,4r)-4-(3-chloro-4-cyanophenoxy)cyclohexyl)-4-(3-oxopropyl)benzamide (may be prepared as described in Description 106; 37.3 mg, 0.091 mmol) was dissolved in DCM (3 mL) and stirred at room temperature for 10 min. triethylamine (0.025 mL, 0.182 mmol) and sodium triacetoxyborohydride (57.7 mg, 0.272 mmol) were then added to the reaction mixture and stirring was continued at room temperature for 3 h. The reaction mixture was quenched with aqueous sodium bicarbonate solution (10 mL) and extracted with DCM (3×10 mL). The combined organic extracts were dried and evaporated. The residue was chromatographed [0-100% ethanol/DCM] then purified by high pH MDAP to give the title compound as an off white solid (21 mg, 0.025 mmol, 27% yield). LCMS (high pH A): Rt=1.23 min, MH⁺=846.

### Example AR86

### N-((1r,4r)-4-(3-Chloro-4-cyanophenoxy)cyclohexyl)-4-(6-(1-(4-(2,4-dioxotetrahydropyrimidin-1(2H)-yl)-1-isopropyl-1H-indole-6-carbonyl)-1,8-diazaspiro[4.5]decan-8-yl)hexyl)benzamide (EAR86)

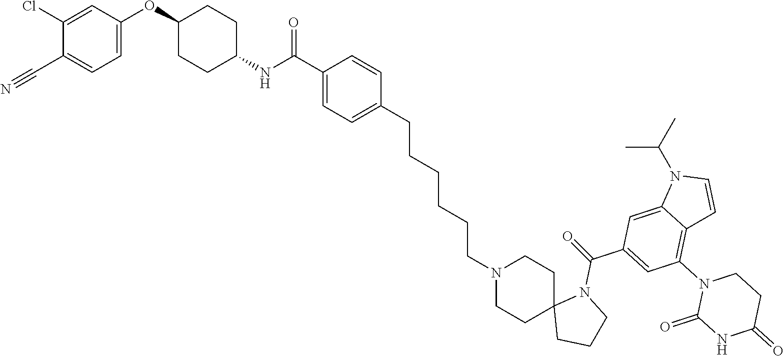 [SMILES: CC(C)n1ccc2c(N3CCC(=O)NC3=O)cc(C(=O)N3CCCC34CCN(CCCCCCc3ccc(C(=O)N[C@H]5CC[C@H](Oc6ccc(C#N)c(Cl)c6)CC5)cc3)CC4)cc21]

1-(1-Isopropyl-6-(2,8-diazaspiro[4.5]decane-2-carbonyl)-1H-indol-4-yl)dihydropyrimidine-2,4(1H,3H)-dione (may be prepared as described in Example CB14; 35 mg, 0.080 mmol) and N-((1r,4r)-4-(3-chloro-4-cyanophenoxy)cyclohexyl)-4-(6-oxohexyl)benzamide (may be prepared as described in Description 108; 36.2 mg, 0.080 mmol) was dissolved in DCM (3 mL) and stirred at room temperature for 10 min. Triethylamine (0.022 mL, 0.160 mmol) and sodium triacetoxyborohydride (50.9 mg, 0.240 mmol) were then added to the reaction mixture and stirring was continued at room temperature for 3 h. The reaction mixture was quenched with aqueous sodium bicarbonate solution (10 mL) and extracted with DCM (3×10 mL). The combined organic extracts were dried and evaporated. The residue was chromatographed [0-100% ethanol/DCM] then purified by MDAP (high pH) to give the title compound as an off white solid (21 mg, 0.024 mmol, 30% yield). LCMS (high pH A): Rt=1.32 min, MH⁺=874.

### Example AR87

### N-((1r,3r)-3-(4-Cyano-3-(trifluoromethyl)phenoxy)-2,2,4,4-tetramethylcyclobutyl)-4-(6-(4-(4-(2,4-dioxotetrahydropyrimidin-1(2H)-yl)-1-isopropyl-1H-indole-6-carbonyl)piperazin-1-yl)hexyl)benzamide (EAR87)

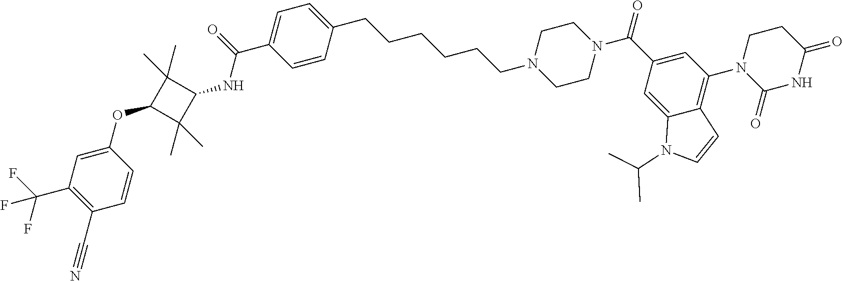 [SMILES: CC(C)n1ccc2c(N3CCC(=O)NC3=O)cc(C(=O)N3CCN(CCCCCCc4ccc(C(=O)N[C@H]5C(C)(C)[C@H](Oc6ccc(C#N)c(C(F)(F)F)c6)C5(C)C)cc4)CC3)cc21]

A mixture of 6-(4-(((1r,3r)-3-(4-cyano-3-(trifluoromethyl)phenoxy)-2,2,4,4-tetramethylcyclobutyl)carbamoyl)phenyl)hexyl 4-methylbenzenesulfonate (may be prepared as described in Description 111; 75 mg, 0.112 mmol), 1-(1-isopropyl-6-(piperazine-1-carbonyl)-1H-indol-4-yl)dihydropyrimidine-2,4(1H,3H)-dione (may be prepared as described in Example CB11; 42.9 mg, 0.112 mmol), N-methylmorpholine (0.037 mL, 0.335 mmol) and sodium iodide (1.676 mg, 0.011 mmol) in DMF (2 mL) was stirred at 80° C. for 2 h. The reaction was purified by MDAP (formic) to give the title compound (29 mg, 0.033 mmol, 29% yield) as a white solid. LCMS (formic A): Rt=1.02 min, MH⁺=882.

### Example AR88

### N-((1′-(4-(3-(4-Cyano-3-(trifluoromethyl)phenyl)-5,5-dimethyl-4-oxo-2-thioxoimidazolidin-1-yl)phenyl)-[1,4′-bipiperidin]-4-yl)methyl)-4-(2,4-dioxotetrahydropyrimidin-1(2H)-yl)-1-isopropyl-N-methyl-1H-indole-6-carboxamide formate (EAR88)

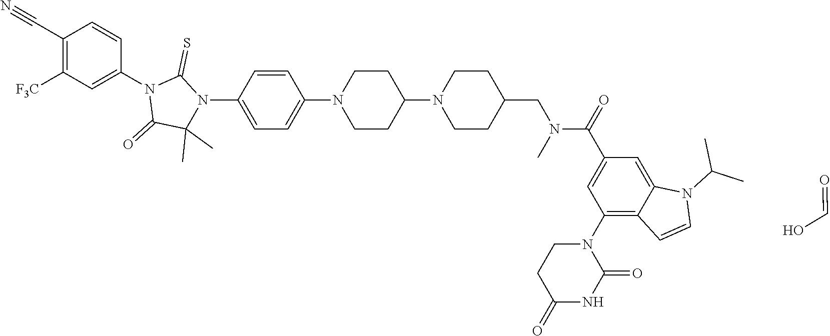 [SMILES: CC(C)n1ccc2c(N3CCC(=O)NC3=O)cc(C(=O)N(C)CC3CCN(C4CCN(c5ccc(N6C(=S)N(c7ccc(C#N)c(C(F)(F)F)c7)C(=O)C6(C)C)cc5)CC4)CC3)cc21 ; O=CO]

To a stirring solution of 1-(4-(3-(4-cyano-3-(trifluoromethyl)phenyl)-5,5-dimethyl-4-oxo-2-thioxoimidazolidin-1-yl)phenyl)piperidin-4-yl 4-methylbenzenesulfonate (may be prepared as described in Description 136; 93 mg, 0.145 mmol) and sodium bicarbonate (36.5 mg, 0.434 mmol) in DMF (0.8 mL) was added 4-(2,4-dioxotetrahydropyrimidin-1(2H)-yl)-1-isopropyl-N-methyl-N-(piperidin-4-ylmethyl)-1H-indole-6-carboxamide (may be prepared as described in Example CBL7) (61.6 mg, 0.145 mmol), followed by stirring whist heating to 80° C. for 40 h. The cooled mixture was filtered through cotton wool and the filtrate was purified by MDAP (formic) and the desired fractions were combined and concentrated in vacuo to give the title compound (12.9 mg, 0.014 mmol, 9% yield) as a pale yellow coloured solid. LCMS (formic A): Rt=1.33 min, MH⁺=896.

### Example AR89

### N-((1-((1-(4-(3-(4-Cyano-3-(trifluoromethyl)phenyl)-5,5-dimethyl-4-oxo-2-thioxoimidazolidin-1-yl)phenyl)piperidin-4-yl)methyl)piperidin-4-yl)methyl)-4-(2,4-dioxotetrahydropyrimidin-1(2H)-yl)-1-isopropyl-N-methyl-1H-indole-6-carboxamide (EAR89)

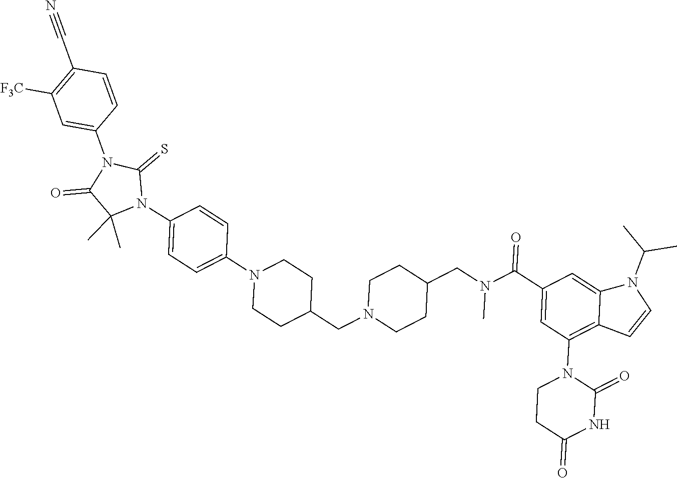 [SMILES: CC(C)n1ccc2c(N3CCC(=O)NC3=O)cc(C(=O)N(C)CC3CCN(CC4CCN(c5ccc(N6C(=S)N(c7ccc(C#N)c(C(F)(F)F)c7)C(=O)C6(C)C)cc5)CC4)CC3)cc21]

To a stirring solution of (1-(4-(3-(4-cyano-3-(trifluoromethyl)phenyl)-5,5-dimethyl-4-oxo-2-thioxoimidazolidin-1-yl)phenyl)piperidin-4-yl)methyl 4-methylbenzenesulfonate (may be prepared as described in Description 137; 55 mg, 0.084 mmol) and sodium bicarbonate (21.11 mg, 0.251 mmol) in DMF (0.8 mL) was added 4-(2,4-dioxotetrahydropyrimidin-1(2H)-yl)-1-isopropyl-N-methyl-N-(piperidin-4-ylmethyl)-1H-indole-6-carboxamide (may be prepared as described in Example CBL7; 35.6 mg, 0.084 mmol), followed by stirring whist heating to 60° C. for 18 h. The solution was cooled to room temperature, filtered through cotton wool, and the solution was purified directly by MDAP (high pH), then repurified using MDAP (high pH) and the desired fractions were combined and concentrated in vacuo to give the title compound (9.4 mg, 10.33 μmol, 12% yield). LCMS (formic A): Rt=1.36 min, MH⁺=910.

### Example AR90

### N-(1-((1-(4-(3-(4-Cyano-3-(trifluoromethyl)phenyl)-5,5-dimethyl-4-oxo-2-thioxoimidazolidin-1-yl)phenyl)piperidin-4-yl)methyl)piperidin-4-yl)-4-(2,4-dioxotetrahydropyrimidin-1(2H)-yl)-1-isopropyl-N-methyl-1H-indole-6-carboxamide formate (EAR90)

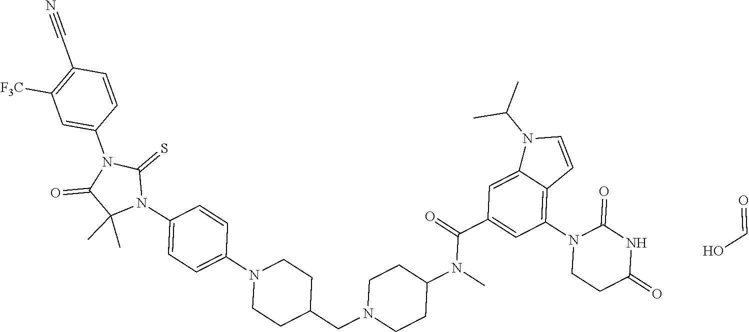 [SMILES: CC(C)n1ccc2c(N3CCC(=O)NC3=O)cc(C(=O)N(C)C3CCN(CC4CCN(c5ccc(N6C(=S)N(c7ccc(C#N)c(C(F)(F)F)c7)C(=O)C6(C)C)cc5)CC4)CC3)cc21 ; O=CO]

To a stirring solution of (1-(4-(3-(4-cyano-3-(trifluoromethyl)phenyl)-5,5-dimethyl-4-oxo-2-thioxoimidazolidin-1-yl)phenyl)piperidin-4-yl)methyl 4-methylbenzenesulfonate (may be prepared as described in Description 137; 47 mg, 0.072 mmol) and sodium bicarbonate (18.04 mg, 0.215 mmol) in DMF (0.8 mL) was added 4-(2,4-dioxotetrahydropyrimidin-1(2H)-yl)-1-isopropyl-N-methyl-N-(piperidin-4-yl)-1H-indole-6-carboxamide (may be prepared as described in Example CBL6; 29.4 mg, 0.072 mmol), followed by stirring whist heating to 60° C. for 18 h. The solution was cooled to room temperature, filtered through cotton wool, and the solution was purified directly by MDAP (formic), and the desired fractions were combined and concentrated in vacuo to give the title compound (9.6 mg, 10.19 μmol, 14% yield). LCMS (high pH A): Rt=0.93 min, MH⁺=896.

### Example AR91

### N-((1-(2-(1-(4-(3-(4-Cyano-3-(trifluoromethyl)phenyl)-5,5-dimethyl-4-oxo-2-thioxoimidazolidin-1-yl)phenyl)piperidin-4-yl)ethyl)piperidin-4-yl)methyl)-4-(2,4-dioxotetrahydropyrimidin-1(2H)-yl)-1-isopropyl-N-methyl-1H-indole-6-carboxamide (EAR91)

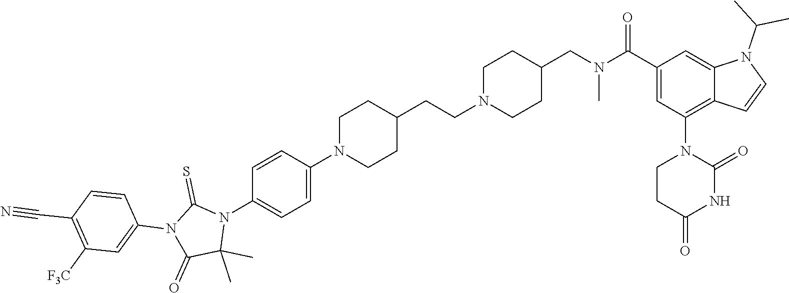 [SMILES: CC(C)n1ccc2c(N3CCC(=O)NC3=O)cc(C(=O)N(C)CC3CCN(CCC4CCN(c5ccc(N6C(=S)N(c7ccc(C#N)c(C(F)(F)F)c7)C(=O)C6(C)C)cc5)CC4)CC3)cc21]

To a stirring solution of 2-(1-(4-(3-(4-cyano-3-(trifluoromethyl)phenyl)-5,5-dimethyl-4-oxo-2-thioxoimidazolidin-1-yl)phenyl)piperidin-4-yl)ethyl 4-methylbenzenesulfonate (may be prepared as described in Description 138; 45 mg, 0.067 mmol) and sodium bicarbonate (16.91 mg, 0.201 mmol) in DMF (0.8 mL) was added 4-(2,4-dioxotetrahydropyrimidin-1(2H)-yl)-1-isopropyl-N-methyl-N-(piperidin-4-ylmethyl)-1H-indole-6-carboxamide (may be prepared as described in Example CBL7; 28.5 mg, 0.067 mmol), followed by stirring whist heating to 60° C. for 16 h. The solution was cooled to room temperature, filtered through cotton wool, and the solution was purified directly by MDAP (high pH), and the desired fractions were combined and concentrated in vacuo to give the title compound (16.1 mg, 0.017 mmol, 26% yield). LCMS (high pH A): Rt=1.34 min, MH⁺=924.

### Example AR92

### 4-(3-(4-(4-(4-(4-(2,4-Dioxotetrahydropyrimidin-1(2H)-yl)-1-isopropyl-1H-indole-6-carbonyl)piperazin-1-yl)butoxy)phenyl)-4,4-dimethyl-5-oxo-2-thioxoimidazolidin-1-yl)-2-(trifluoromethyl)benzonitrile (EAR92)

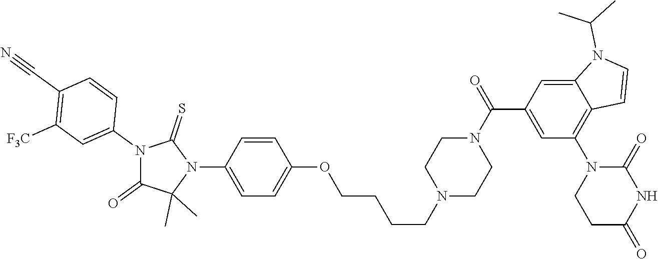 [SMILES: CC(C)n1ccc2c(N3CCC(=O)NC3=O)cc(C(=O)N3CCN(CCCCOc4ccc(N5C(=S)N(c6ccc(C#N)c(C(F)(F)F)c6)C(=O)C5(C)C)cc4)CC3)cc21]

To a stirring solution of 4-(4-(3-(4-cyano-3-(trifluoromethyl)phenyl)-5,5-dimethyl-4-oxo-2-thioxoimidazolidin-1-yl)phenoxy)butyl 4-methylbenzenesulfonate (may be prepared as described in Description 142; 41 mg, 0.065 mmol) and sodium bicarbonate (16.36 mg, 0.195 mmol) in DMF (0.8 mL) was added 1-(1-isopropyl-6-(piperazine-1-carbonyl)-1H-indol-4-yl)dihydropyrimidine-2,4(1H,3H)-dione (may be prepared as described in Example CB11; 24.89 mg, 0.065 mmol), followed by stirring whist heating to 60° C. for 16 h. The solution was cooled to room temperature and the solution was purified directly by MDAP (high pH), and the desired fractions were combined and concentrated in vacuo to give the title compound (10.3 mg, 0.012 mmol, 19% yield). LCMS (high pH A): Rt=1.24 min, MH⁺=843.

### Example AR93

### N-((1-(3-(4-(3-(4-Cyano-3-(trifluoromethyl)phenyl)-5,5-dimethyl-4-oxo-2-thioxoimidazolidin-1-yl)phenoxy)propyl)piperidin-4-yl)methyl)-4-(2,4-dioxotetrahydropyrimidin-1(2H)-yl)-1-isopropyl-N-methyl-1H-indole-6-carboxamide formate (EAR93)

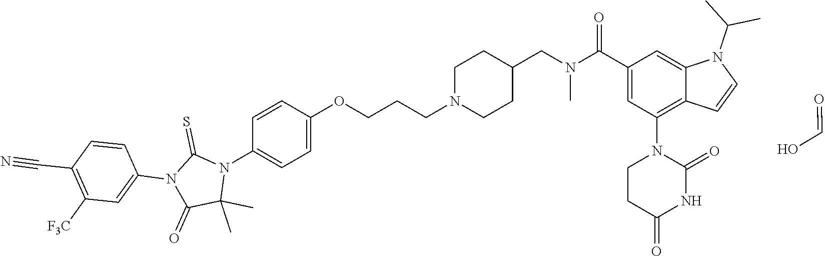 [SMILES: CC(C)n1ccc2c(N3CCC(=O)NC3=O)cc(C(=O)N(C)CC3CCN(CCCOc4ccc(N5C(=S)N(c6ccc(C#N)c(C(F)(F)F)c6)C(=O)C5(C)C)cc4)CC3)cc21 ; O=CO]

To a stirring solution of 3-(4-(3-(4-cyano-3-(trifluoromethyl)phenyl)-5,5-dimethyl-4-oxo-2-thioxoimidazolidin-1-yl)phenoxy)propyl 4-methylbenzenesulfonate (may be prepared as described in Description 141; 41 mg, 0.066 mmol) and sodium bicarbonate (16.73 mg, 0.199 mmol) in DMF (0.8 mL) was added 4-(2,4-dioxotetrahydropyrimidin-1(2H)-yl)-1-isopropyl-N-methyl-N-(piperidin-4-ylmethyl)-1H-indole-6-carboxamide (may be prepared as described in Example CBL7; 28.2 mg, 0.066 mmol), followed by stirring whist heating to 60° C. for 16 h. The solution was cooled to room temperature and the solution was purified directly by MDAP (formic), and the desired fractions were combined and concentrated in vacuo to give the title compound (15.9 mg, 0.017 mmol, 26% yield). LCMS (high pH A): Rt=0.88 min, MH⁺=871.

### Example AR94

### 4-(3-(4-(3-(4-(4-(2,4-Dioxotetrahydropyrimidin-1(2H)-yl)-1-isopropyl-1H-indole-6-carbonyl)piperazin-1-yl) propoxy)phenyl)-4,4-dimethyl-5-oxo-2-thioxoimidazolidin-1-yl)-2-(trifluoromethyl)benzonitrile formate (EAR94)

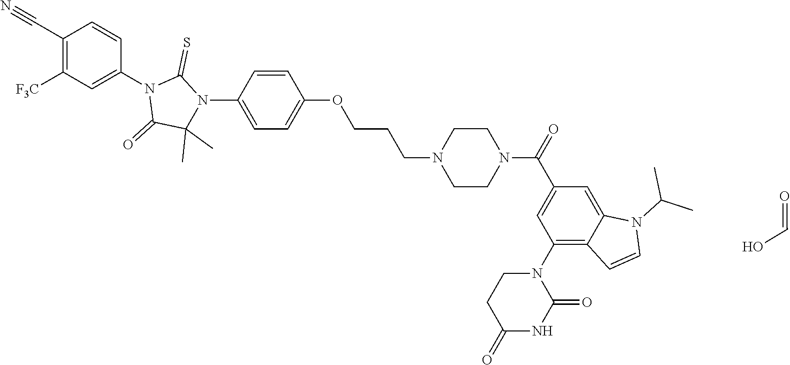 [SMILES: CC(C)n1ccc2c(N3CCC(=O)NC3=O)cc(C(=O)N3CCN(CCCOc4ccc(N5C(=S)N(c6ccc(C#N)c(C(F)(F)F)c6)C(=O)C5(C)C)cc4)CC3)cc21 ; O=CO]

To a stirring solution of 3-(4-(3-(4-cyano-3-(trifluoromethyl)phenyl)-5,5-dimethyl-4-oxo-2-thioxoimidazolidin-1-yl)phenoxy)propyl 4-methylbenzenesulfonate (may be prepared as described in Description 141; 41 mg, 0.066 mmol) and sodium bicarbonate (16.73 mg, 0.199 mmol) in DMF (0.8 mL) was added 1-(1-isopropyl-6-(piperazine-1-carbonyl)-1H-indol-4-yl)dihydropyrimidine-2,4(1H,3H)-dione (may be prepared as described in Example CB11; 25.5 mg, 0.066 mmol), followed by stirring whist heating to 60° C. for 16 h. The solution was cooled to room temperature and the solution was purified directly by MDAP (formic), and the desired fractions were combined and concentrated in vacuo to give the title compound (13.5 mg, 0.015 mmol, 23% yield). LCMS (high pH A): Rt=0.86 min, MH⁺=829.

### Example AR95

### 4-(3-(4-(4-((4-(4-(2,4-Dioxotetrahydropyrimidin-1(2H)-yl)-1H-indazol-1-yl)piperidin-1-yl)methyl)piperidin-1-yl)phenyl)-4,4-dimethyl-5-oxo-2-thioxoimidazolidin-1-yl)-2-(trifluoromethyl)benzonitrile (EAR95)

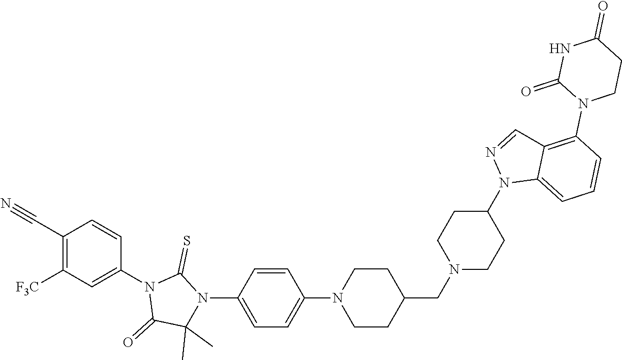 [SMILES: CC1(C)C(=O)N(c2ccc(C#N)c(C(F)(F)F)c2)C(=S)N1c1ccc(N2CCC(CN3CCC(n4ncc5c(N6CCC(=O)NC6=O)cccc54)CC3)CC2)cc1]

To a stirring mixture of (1-(4-(3-(4-cyano-3-(trifluoromethyl)phenyl)-5,5-dimethyl-4-oxo-2-thioxoimidazolidin-1-yl)phenyl)piperidin-4-yl)methyl 4-methylbenzenesulfonate (may be prepared as described in Description 137; 51 mg, 0.078 mmol) and sodium bicarbonate (19.6 mg, 0.233 mmol) in DMF (0.8 mL) was added 1-(1-(piperidin-4-yl)-1H-indazol-4-yl)dihydropyrimidine-2,4(1H,3H)-dione trifluoroacetate (may be prepared as described in Example CB4; 24.3 mg, 0.078 mmol), followed by stirring whist heating to 60° C. for 18 h. The solution was cooled to room temperature, filtered through cotton wool, and the solution was purified directly by MDAP (formic), then repurified by MDAP (high pH), to give the title compound (5.6 mg, 7.02 μmol, 94% yield). LCMS (high pH A): Rt=1.34 min, MH⁼=798

### Example AR96

### 4-(3-(4-(4-(4-(2,4-Dioxotetrahydropyrimidin-1(2H)-yl)-1H-pyrrolo[2,3-c]pyridin-1-yl)-[1,4′-bipiperidin]-1′-yl)phenyl)-4,4-dimethyl-5-oxo-2-thioxoimidazolidin-1-yl)-2-(trifluoromethyl)benzonitrile formate (EAR96)

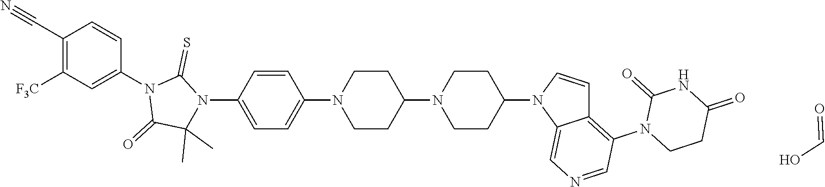 [SMILES: CC1(C)C(=O)N(c2ccc(C#N)c(C(F)(F)F)c2)C(=S)N1c1ccc(N2CCC(N3CCC(n4ccc5c(N6CCC(=O)NC6=O)cncc54)CC3)CC2)cc1 ; O=CO]

To a stirring mixture of 1-(4-(3-(4-cyano-3-(trifluoromethyl)phenyl)-5,5-dimethyl-4-oxo-2-thioxoimidazolidin-1-yl)phenyl)piperidin-4-yl 4-methylbenzenesulfonate (may be prepared as described in Description 136; 120 mg, 0.187 mmol) and sodium bicarbonate (47.1 mg, 0.560 mmol) in DMF (0.8 mL) was added 1-(1-(piperidin-4-yl)-1H-pyrrolo[2,3-c]pyridin-4-yl)dihydropyrimidine-2,4(1H,3H)-dione (may be prepared as described in Example CB6; 58.5 mg, 0.187 mmol), followed by stirring whist heating to 80° C. for 40 h. The cooled mixture was diluted with DMSO (1 mL) and MeOH (1 mL), and filtered through cotton wool and the filtrate was purified by formic HPLC, eluting with 0-30% (MeCN+1% formic acid):(water+1% formic acid), and the desired fractions were combined and concentrated in vacuo to give the title compound as a white coloured solid (8.9 mg, 10.72 μmol, 6% yield). LCMS (formic A): Rt=0.66 min, MH⁺=784.

### Example AR97

### 4-(3-(4-(4-(2-(4-(4-(2,4-Dioxotetrahydropyrimidin-1(2H)-yl)-1H-pyrazolo[3,4-c]pyridin-1-yl)piperidin-1-yl)ethyl)piperidin-1-yl)phenyl)-4,4-dimethyl-5-oxo-2-thioxoimidazolidin-1-yl)-2-(trifluoromethyl)benzonitrile formate (EAR97)

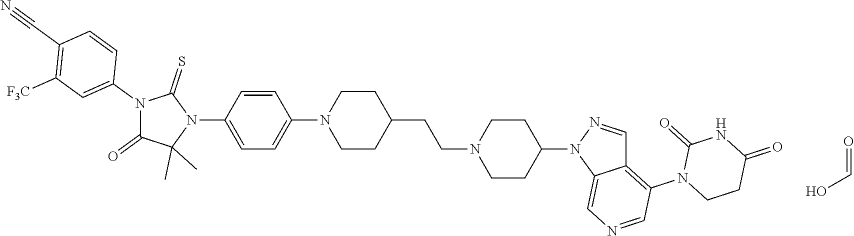 [SMILES: CC1(C)C(=O)N(c2ccc(C#N)c(C(F)(F)F)c2)C(=S)N1c1ccc(N2CCC(CCN3CCC(n4ncc5c(N6CCC(=O)NC6=O)cncc54)CC3)CC2)cc1 ; O=CO]

To a stirring mixture of 2-(1-(4-(3-(4-cyano-3-(trifluoromethyl)phenyl)-5,5-dimethyl-4-oxo-2-thioxoimidazolidin-1-yl)phenyl)piperidin-4-yl)ethyl 4-methylbenzenesulfonate (may be prepared as described in Description 138; 47 mg, 0.070 mmol) and sodium bicarbonate (17.66 mg, 0.210 mmol) in DMF (0.8 mL) was added 1-(1-(piperidin-4-yl)-1H-pyrazolo[3,4-c]pyridin-4-yl)dihydropyrimidine-2,4(1H,3H)-dione (may be prepared as described in Example CB8; 22.0 mg, 0.070 mmol), followed by stirring whist heating to 60° C. for 16 h. The solution was cooled to room temperature, filtered through cotton wool, and the solution was purified directly by MDAP (formic), and then repurified using the same conditions, and desired fractions were combined and concentrated in vacuo to give the title compound (7.0 mg, 8.15 μmol, 12% yield). LCMS (formic A): Rt=0.95 min, MH⁺=813

### Example AR98

### 4-(3-(4-(4-((4-(4-(2,4-Dioxotetrahydropyrimidin-1(2H)-yl)-1H-pyrazolo[3,4-c]pyridin-1-yl)piperidin-1-yl)methyl)piperidin-1-yl)phenyl)-4,4-dimethyl-5-oxo-2-thioxoimidazolidin-1-yl)-2-(trifluoromethyl)benzonitrile (EAR98)

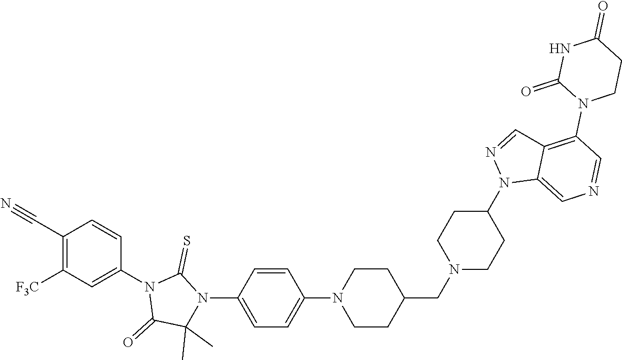 [SMILES: CC1(C)C(=O)N(c2ccc(C#N)c(C(F)(F)F)c2)C(=S)N1c1ccc(N2CCC(CN3CCC(n4ncc5c(N6CCC(=O)NC6=O)cncc54)CC3)CC2)cc1]

To a stirring mixture of (1-(4-(3-(4-cyano-3-(trifluoromethyl)phenyl)-5,5-dimethyl-4-oxo-2-thioxoimidazolidin-1-yl)phenyl)piperidin-4-yl)methyl 4-methylbenzenesulfonate (may be prepared as described in Description 137; 53 mg, 0.081 mmol) and sodium bicarbonate (20.3 mg, 0.242 mmol) in DMF (0.8 mL) was added 1-(1-(piperidin-4-yl)-1H-pyrazolo[3,4-c]pyridin-4-yl)dihydropyrimidine-2,4(1H,3H)-dione (may be prepared as described in Example CB8; 25.4 mg, 0.081 mmol), followed by stirring whist heating to 60° C. for 18 h. The solution was cooled to room temperature, filtered through cotton wool, and the solution was purified directly by MDAP (high pH), and the desired fractions were combined and concentrated in vacuo to give the title compound (17.2 mg, 0.022 mmol, 27% yield). LCMS (high pH A): Rt=1.25 min, MH⁺=799

### Example AR99

### 4-(3-(4-(4-((4-(4-(2,4-Dioxotetrahydropyrimidin-1(2H)-yl)-1H-indol-1-yl)piperidin-1-yl)methyl)piperidin-1-yl)phenyl)-4,4-dimethyl-5-oxo-2-thioxoimidazolidin-1-yl)-2-(trifluoromethyl)benzonitrile formate. (EAR99)

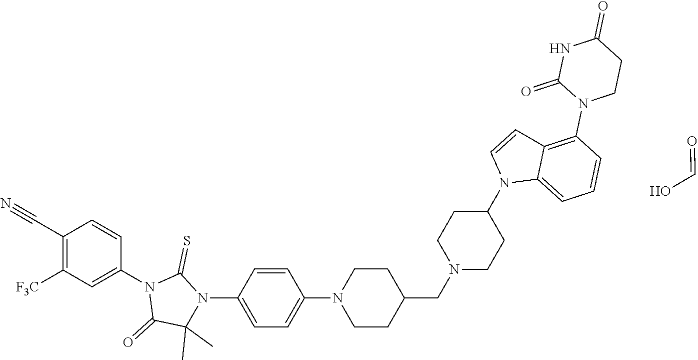 [SMILES: CC1(C)C(=O)N(c2ccc(C#N)c(C(F)(F)F)c2)C(=S)N1c1ccc(N2CCC(CN3CCC(n4ccc5c(N6CCC(=O)NC6=O)cccc54)CC3)CC2)cc1 ; O=CO]

To a stirring mixture of (1-(4-(3-(4-cyano-3-(trifluoromethyl)phenyl)-5,5-dimethyl-4-oxo-2-thioxoimidazolidin-1-yl)phenyl)piperidin-4-yl)methyl 4-methylbenzenesulfonate (may be prepared as described in Description 137; 49 mg, 0.075 mmol) and sodium bicarbonate (18.8 mg, 0.224 mmol) in DMF (0.8 mL) was added 1-(1-(piperidin-4-yl)-1H-indol-4-yl)dihydropyrimidine-2,4(1H,3H)-dione (may be prepared as described in Example CB1; 23.3 mg, 0.075 mmol), followed by stirring whist heating to 60° C. for 16 h. The solution was cooled to room temperature, filtered through cotton wool, and the solution was purified directly by MDAP (high pH), then repurified by MDAP (formic) and the desired fractions were combined and concentrated in vacuo to give the title compound (9.4 mg, 0.011 mmol, 15% yield). LCMS (formic A): Rt=0.91 min, MH⁺=797

### Example AR100

### 4-(3-(4-(4-(2-(4-(4-(2,4-Dioxotetrahydropyrimidin-1(2H)-yl)-1H-pyrrolo[2,3-c]pyridin-1-yl)piperidin-1-yl)ethyl)piperidin-1-yl)phenyl)-4,4-dimethyl-5-oxo-2-thioxoimidazolidin-1-yl)-2-(trifluoromethyl)benzonitrile formate (EAR100)

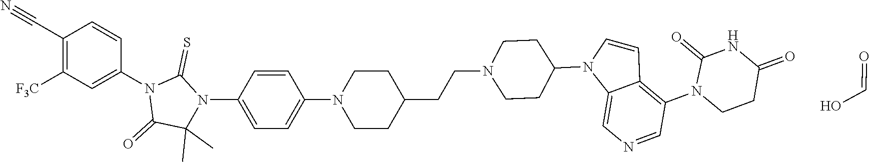 [SMILES: CC1(C)C(=O)N(c2ccc(C#N)c(C(F)(F)F)c2)C(=S)N1c1ccc(N2CCC(CCN3CCC(n4ccc5c(N6CCC(=O)NC6=O)cncc54)CC3)CC2)cc1 ; O=CO]

To a stirring mixture of 2-(1-(4-(3-(4-cyano-3-(trifluoromethyl)phenyl)-5,5-dimethyl-4-oxo-2-thioxoimidazolidin-1-yl)phenyl)piperidin-4-yl)ethyl 4-methylbenzenesulfonate (may be prepared as described in Description 138; 45 mg, 0.067 mmol) and sodium bicarbonate (16.9 mg, 0.201 mmol) in DMF (0.8 mL) was added 1-(1-(piperidin-4-yl)-1H-pyrrolo[2,3-c]pyridin-4-yl)dihydropyrimidine-2,4(1H,3H)-dione (may be prepared as described in Example CB6; 21.0 mg, 0.067 mmol), followed by stirring whist heating to 60° C. for 16 h. The solution was cooled to room temperature, filtered through cotton wool, and the solution was purified directly by MDAP (high pH), then repurified using MDAP (formic pH), to give the title compound (5.8 mg, 6.76 μmol, 10% yield). LCMS (formic A): Rt=0.68 min, MH⁺=812

### Example AR101

### 4-(3-(4-(4-((4-(4-(2,4-Dioxotetrahydropyrimidin-1(2H)-yl)-1H-pyrrolo[2,3-c]pyridin-1-yl)piperidin-1-yl)methyl)piperidin-1-yl)phenyl)-4,4-dimethyl-5-oxo-2-thioxoimidazolidin-1-yl)-2-(trifluoromethyl)benzonitrile (EAR101)

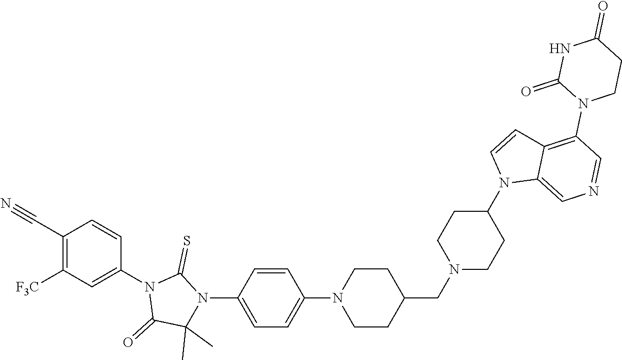 [SMILES: CC1(C)C(=O)N(c2ccc(C#N)c(C(F)(F)F)c2)C(=S)N1c1ccc(N2CCC(CN3CCC(n4ccc5c(N6CCC(=O)NC6=O)cncc54)CC3)CC2)cc1]

To a stirring mixture of (1-(4-(3-(4-cyano-3-(trifluoromethyl)phenyl)-5,5-dimethyl-4-oxo-2-thioxoimidazolidin-1-yl)phenyl)piperidin-4-yl)methyl 4-methylbenzenesulfonate (may be prepared as described in Description 137; 49 mg, 0.075 mmol) and sodium bicarbonate (18.8 mg, 0.224 mmol) in DMF (0.8 mL) was added 1-(1-(piperidin-4-yl)-1H-pyrrolo[2,3-c]pyridin-4-yl)dihydropyrimidine-2,4(1H,3H)-dione (may be prepared as described in Example CB6; 23.4 mg, 0.075 mmol), followed by stirring whist heating to 60° C. for 16 h. The solution was cooled to room temperature, filtered through cotton wool, and the solution was purified directly by MDAP (high pH), and the desired fractions were combined and concentrated in vacuo to give the title compound (19.9 mg, 0.025 mmol, 33% yield). LCMS (high pH A): Rt=1.25 min, MH⁺=798

### Example AR102

### 4-(3-(4-(4-(4-(4-(2,4-Dioxotetrahydropyrimidin-1(2H)-yl)-1H-pyrrolo[2,3-c]pyridin-1-yl)piperidin-1-yl)butoxy)phenyl)-4,4-dimethyl-5-oxo-2-thioxoimidazolidin-1-yl)-2-(trifluoromethyl)benzonitrile formate (EAR102)

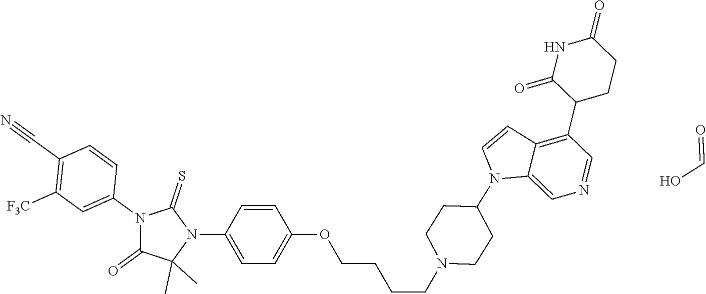 [SMILES: CC1(C)C(=O)N(c2ccc(C#N)c(C(F)(F)F)c2)C(=S)N1c1ccc(OCCCCN2CCC(n3ccc4c(C5CCC(=O)NC5=O)cncc43)CC2)cc1 ; O=CO]

To a stirring mixture of 4-(4-(3-(4-cyano-3-(trifluoromethyl)phenyl)-5,5-dimethyl-4-oxo-2-thioxoimidazolidin-1-yl)phenoxy)butyl 4-methylbenzenesulfonate (may be prepared as described in Description 142; 40 mg, 0.063 mmol) and sodium bicarbonate (16.0 mg, 0.190 mmol) in DMF (0.8 mL) was added 1-(1-(piperidin-4-yl)-1H-pyrrolo[2,3-c]pyridin-4-yl)dihydropyrimidine-2,4(1H,3H)-dione (may be prepared as described in Example CB6; 19.8 mg, 0.063 mmol), followed by stirring whist heating to 60° C. for 16 h. The solution was cooled to room temperature and the solution was purified directly by MDAP (formic), and the desired fractions were combined and concentrated in vacuo to give the title compound (14.3 mg, 0.017 mmol, 28% yield). LCMS (formic A): Rt=0.66 min, MH⁺=773

### Example AR103

### 4-(3-(4-(3-(4-(4-(2,4-Dioxotetrahydropyrimidin-1(2H)-yl)-1H-indol-1-yl)piperidin-1-yl) propoxy)phenyl)-4,4-dimethyl-5-oxo-2-thioxoimidazolidin-1-yl)-2-(trifluoromethyl)benzonitrile formate (EAR103)

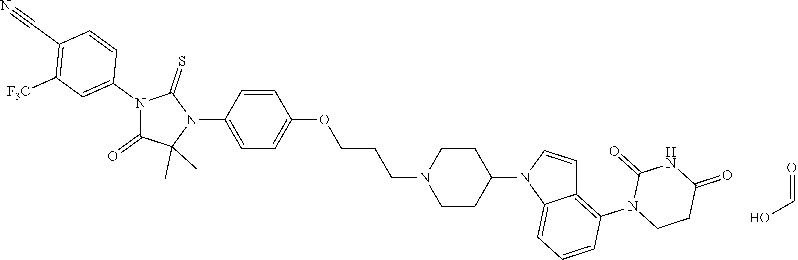 [SMILES: CC1(C)C(=O)N(c2ccc(C#N)c(C(F)(F)F)c2)C(=S)N1c1ccc(OCCCN2CCC(n3ccc4c(N5CCC(=O)NC5=O)cccc43)CC2)cc1 ; O=CO]

To a stirring mixture of 3-(4-(3-(4-cyano-3-(trifluoromethyl)phenyl)-5,5-dimethyl-4-oxo-2-thioxoimidazolidin-1-yl)phenoxy)propyl 4-methylbenzenesulfonate (may be prepared as described in Description 141; 42 mg, 0.068 mmol) and sodium bicarbonate (17.1 mg, 0.204 mmol) in DMF (0.8 mL) was added 1-(1-(piperidin-4-yl)-1H-indol-4-yl)dihydropyrimidine-2,4(1H,3H)-dione (may be prepared as described in Example CB1; 21.2 mg, 0.068 mmol), followed by stirring whist heating to 60° C. for 16 h. The solution was cooled to room temperature and the solution was purified directly by MDAP (formic), and the desired fractions were combined and concentrated in vacuo to give the title compound, (14.2 mg, 0.018 mmol, 26% yield). LCMS (formic A): Rt=0.86 min, MH⁺=758

### Example AR104

### 4-(3-(4-(3-(4-(4-(2,4-Dioxotetrahydropyrimidin-1(2H)-yl)-1H-pyrrolo[2,3-c]pyridin-1-yl)piperidin-1-yl) propoxy)phenyl)-4,4-dimethyl-5-oxo-2-thioxoimidazolidin-1-yl)-2-(trifluoromethyl)benzonitrile (EAR104)

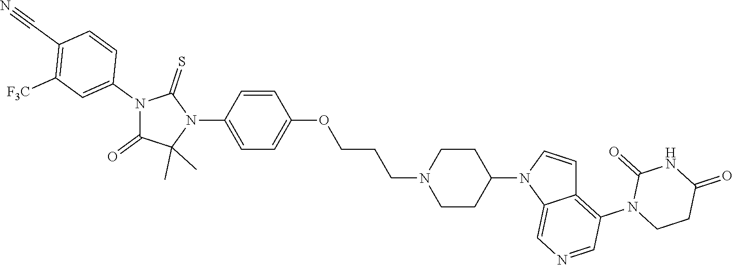 [SMILES: CC1(C)C(=O)N(c2ccc(C#N)c(C(F)(F)F)c2)C(=S)N1c1ccc(OCCCN2CCC(n3ccc4c(N5CCC(=O)NC5=O)cncc43)CC2)cc1]

To a stirring mixture of 3-(4-(3-(4-cyano-3-(trifluoromethyl)phenyl)-5,5-dimethyl-4-oxo-2-thioxoimidazolidin-1-yl)phenoxy)propyl 4-methylbenzenesulfonate (may be prepared as described in Description 141; 87 mg, 0.141 mmol) and sodium bicarbonate (35.5 mg, 0.423 mmol) in DMF (0.8 mL) was added 1-(1-(piperidin-4-yl)-1H-pyrrolo[2,3-c]pyridin-4-yl)dihydropyrimidine-2,4(1H,3H)-dione (may be prepared as described in Example CB6; 44.1 mg, 0.141 mmol), followed by stirring whist heating to 60° C. for 16 h. The crude product was purified directly by MDAP (high pH), and the desired fractions were combined and concentrated in vacuo to give the title compound (20.7 mg, 0.027 mmol, 19% yield) as a white coloured solid. LCMS (high pH A): Rt=1.17 min, MH⁺=759

### Examples AR105-AR107

### General Procedure

(2R,5S)-4-(3-Chloro-4-cyanophenyl)-N-(2-(4-formylpiperidin-1-yl)pyrimidin-5-yl)-2,5-dimethylpiperazine-1-carboxamide (may be prepared as described in Description 163; 70 mg, 0.145 mmol) and the appropriate amine (0.145 mmol) was suspended in DCM (5 mL) and stirred for 30 min at room temperature. Sodium triacetoxyborohydride (61.6 mg, 0.290 mmol) was added and the mixture stirred for 1 h at room temperature. The solution was washed with saturated sodium bicarbonate solution and the organic layer separated with a hydrophobic frit. The solvent was evaporated in vacuo to give the desired products as pale yellow solids.

| Ex- | | | Mass, | |
|---|---|---|---|---|
| ample | Name | Intermediate | yield | Analytical |
| AR105 | 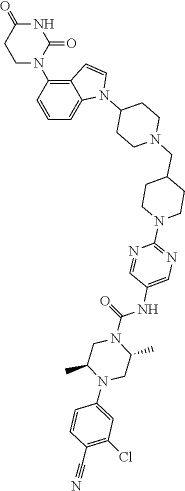 [SMILES: C[C@@H]1CN(c2ccc(C#N)c(Cl)c2)[C@@H](C)CN1C(=O)Nc1cnc(N2CCC(CN3CCC(n4ccc5c(N6CCC(=O)NC6=O)cccc54)CC3)CC2)nc1] | ECB1 | 52 mg, 46% | LCMS (high pH A): Rt = 1.22 min, MH⁺ = 778.2/780.2 (1 Cl) |
| | (2R,5S)-4-(3-Chloro-4-cyanophenyl)-N-(2-(4- | | | |
| | ((4-(4-(2,4-dioxotetrahydropyrimidin-1(2H)- | | | |
| | yl)-1H-indol-1-yl)piperidin-1- | | | |
| | yl)methyl)piperidin-1-yl)pyrimidin-5-yl)-2,5- | | | |
| | dimethylpiperazine-1-carboxamide | | | |
| AR106 | 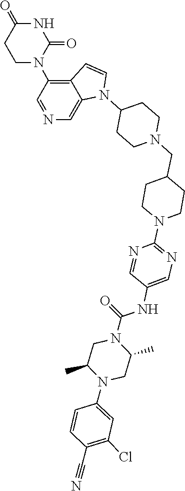 [SMILES: C[C@@H]1CN(c2ccc(C#N)c(Cl)c2)[C@@H](C)CN1C(=O)Nc1cnc(N2CCC(CN3CCC(n4ccc5c(N6CCC(=O)NC6=O)cncc54)CC3)CC2)nc1] | ECB6 | 58 mg, 51% | LCMS (high pH A): Rt = 1.07 min, MH⁺ = 779.3/781.3 (1 Cl) |
| | (2R,5S)-4-(3-Chloro-4-cyanophenyl)-N-(2-(4- | | | |
| | ((4-(4-(2,4-dioxotetrahydropyrimidin-1(2H)- | | | |
| | yl)-1H-pyrrolo[2,3-c]pyridin-1-yl)piperidin-1- | | | |
| | yl)methyl)piperidin-1-yl)pyrimidin-5-yl)-2,5- | | | |
| | dimethylpiperazine-1-carboxamide | | | |
| AR107 | 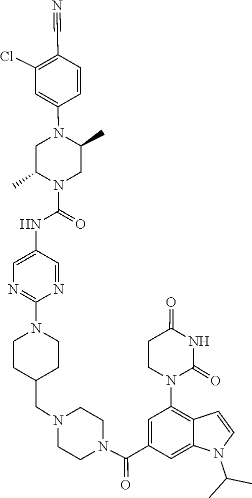 [SMILES: CC(C)n1ccc2c(N3CCC(=O)NC3=O)cc(C(=O)N3CCN(CC4CCN(c5ncc(NC(=O)N6C[C@H](C)N(c7ccc(C#N)c(Cl)c7)C[C@H]6C)cn5)CC4)CC3)cc21] | ECB11 | 65 mg, 53% | LCMS (high pH A) Rt = 1.14 min, MH⁺ = 849.3. 851.3 |
| | (2R,5S)-4-(3-Chloro-4-cyanophenyl)-N-(2-(4- | | | |
| | ((4-(4-(2,4-dioxotetrahydropyrimidin-1(2H)- | | | |
| | yl)-1-isopropyl-1H-indole-6- | | | |
| | carbonyl)piperazin-1-yl)methyl)piperidin-1- | | | |
| | yl)pyrimidin-5-yl)-2,5-dimethylpiperazine-1- | | | |
| | carboxamide | | | |

### Examples AR108-AR113

### General Procedure

The appropriate aldehyde (0.1 mmol) and amine (0.100 mmol) were combined and dissolved in DCM (3 mL). The mixture was stirred for 1 h then sodium triacetoxyborohydride (0.042 g, 0.200 mmol) was added. The mixture was stirred overnight at room temperature and quenched with saturated sodium bicarbonate solution. The mixture was diluted with DCM (5 mL) and MeOH (2 mL) and stirred for 10 min. The layers were separated using hydrophobic frits and the organics dried and evaporated in vacuo. the material was purified by the stated method.

| | | | | Purification |
|---|---|---|---|---|
| | | | | Method |
| Ex- | Name | Alde- | | Mass, |
| ample | Analytical | hyde | Amine | Yield |
| AR108 | 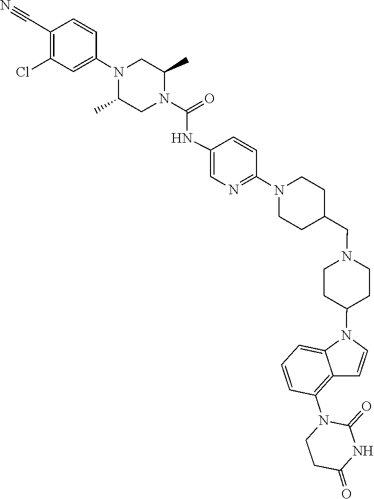 [SMILES: C[C@@H]1CN(c2ccc(C#N)c(Cl)c2)[C@@H](C)CN1C(=O)Nc1ccc(N2CCC(CN3CCC(n4ccc5c(N6CCC(=O)NC6=O)cccc54)CC3)CC2)nc1] | D157 | ECB1 | MDAP (high pH) 9 mg, 10% |
| | (2R,5S)-4-(3-Chloro-4-cyanophenyl)-N-(6-(4-((4- | | | |
| | (4-(2,4-dioxotetrahydropyrimidin- | | | |
| | 1(2H)-yl)-1H-indol-1-yl)piperidin-1- | | | |
| | yl)methyl)piperidin-1-yl)pyridin-3-yl)-2,5-dimethyl | | | |
| | piperazine-1-carboxamide | | | |
| | LCMS (high pH A): Rt = 1.22 min, MH⁺ = 777.3, 779.3 | | | |
| AR109 | 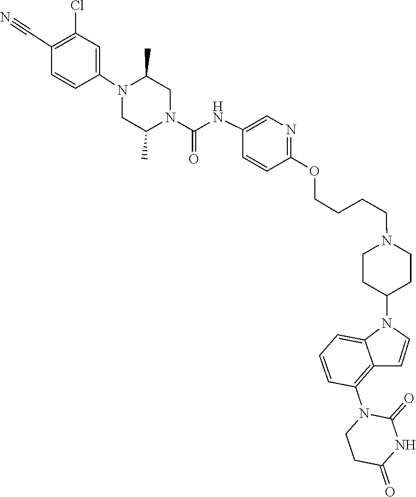 [SMILES: C[C@@H]1CN(c2ccc(C#N)c(Cl)c2)[C@@H](C)CN1C(=O)Nc1ccc(OCCCCN2CCC(n3ccc4c(N5CCC(=O)NC5=O)cccc43)CC2)nc1] | D150 | ECB1 | column chroma- tography on 12 g silica column eluting with 0- 10% 2M MeOH ammonia/ DCM 18 mg, 22% |
| | (2R,5S)-4-(3-Chloro-4-cyanophenyl)-N-(6-(4-(4- | | | |
| | (4-(2,4-dioxotetrahydropyrimidin-1(2H)-yl)-1H- | | | |
| | indol-1-yl)piperidin-1-yl)butoxy)pyridin-3-yl)-2,5- | | | |
| | dimethylpiperazine-1-carboxamide | | | |
| | LCMS (high pH A): Rt = 1.19 min, MH⁺ = 752.2, 754.2 | | | |
| AR110 | 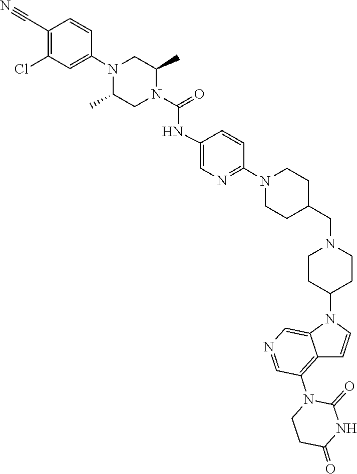 [SMILES: C[C@@H]1CN(c2ccc(C#N)c(Cl)c2)[C@@H](C)CN1C(=O)Nc1ccc(N2CCC(CN3CCC(n4ccc5c(N6CCC(=O)NC6=O)cncc54)CC3)CC2)nc1] | D157 | ECB6 | MDAP (high pH) 12 mg, 15% |
| | (2R,5S)-4-(3-Chloro-4-cyanophenyl)-N-(6-(4- | | | |
| | formylpiperidin-1-yl)pyridin-3-yl)-2,5- | | | |
| | dimethylpiperazine-1-carboxamide | | | |
| | LCMS (high pH A): Rt = 1.08 min, MH⁺ = 788.2, 780.2 | | | |
| AR111 | 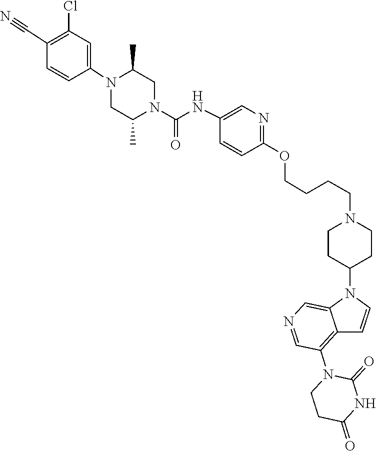 [SMILES: C[C@@H]1CN(c2ccc(C#N)c(Cl)c2)[C@@H](C)CN1C(=O)Nc1ccc(OCCCCN2CCC(n3ccc4c(N5CCC(=O)NC5=O)cncc43)CC2)nc1] | D150 | ECB6 | column chroma- tography on 12 g silica columns eluting with 0- 10% 2M MeOH ammonia/ DCM 35 mg, 44% |
| | (2R,5S)-4-(3-Chloro-4-cyanophenyl)-N-(6-(4-(4- | | | |
| | (4-(2,4-dioxotetrahydropyrimidin-1(2H)-yl)-1H- | | | |
| | pyrrolo[2,3-c]pyridin-1-yl)piperidin-1- | | | |
| | yl)butoxy)pyridin-3-yl)-2,5-dimethylpiperazine-1- | | | |
| | carboxamide | | | |
| | LCMS (high pH A): Rt = 1.06 min, MH⁺ = 753.3, 755.3 | | | |
| AR112 | 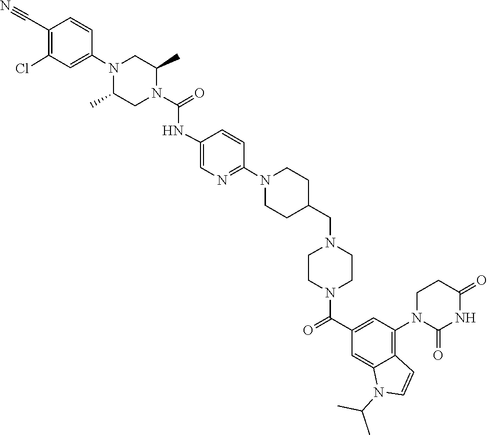 | D157 | ECB11 | MDAP (high pH) 25 mg, 28% |
| | (2R,5S)-4-(3-Chloro-4-cyanophenyl)-N-(6-(4-((4- | | | |
| | (4-(2,4-dioxotetrahydropyrimidin-1(2H)-yl)-1- | | | |
| | isopropyl-1H-indole-6-carbonyl)piperazin-1- | | | |
| | yl)methyl)piperidin-1-yl)pyridin-3-yl)-2,5- | | | |
| | dimethylpiperazine-1-carboxamide | | | |
| | LCMS (high pH A): Rt = 1.14 min, MH⁺ = 848.2, 850.2 | | | |
| AR113 | 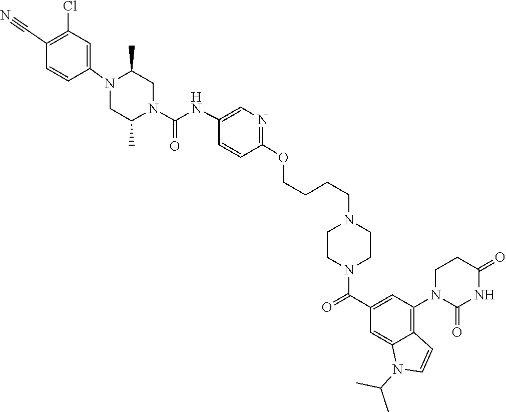 [SMILES: CC(C)n1ccc2c(N3CCC(=O)NC3=O)cc(C(=O)N3CCN(CCCCOc4ccc(NC(=O)N5C[C@H](C)N(c6ccc(C#N)c(Cl)c6)C[C@H]5C)cn4)CC3)cc21] | D150 | ECB11 | column chroma- tography on 12 g silica columns eluting with 0- 10% 2M MeOH ammonia/ DCM 46 mg, 53% |
| | (2R,5S)-4-(3-Chloro-4-cyanophenyl)-N-(6-(4-(4- | | | |
| | (4-(2,4-dioxotetrahydropyrimidin-1(2H)-yl)-1- | | | |
| | isopropyl-1H-indole-6-carbonyl)piperazin-1- | | | |
| | yl)butoxy)pyridin-3-yl)-2,5-dimethylpiperazine-1- | | | |
| | carboxamide | | | |
| | LCMS (high pH A): Rt = 1.12 min, MH⁺ = 823.2, 825.2 | | | |

### Example AR114

### rac-(trans)-4-(3-Chloro-4-cyanophenyl)-N-(2-(4-((4-(4-(2,4-dioxotetrahydropyrimidin-1(2H)-yl)-1H-indol-1-yl)piperidin-1-yl)methyl)piperidin-1-yl)pyrimidin-5-yl)-2,5-dimethylpiperazine-1-carboxamide (EAR114)

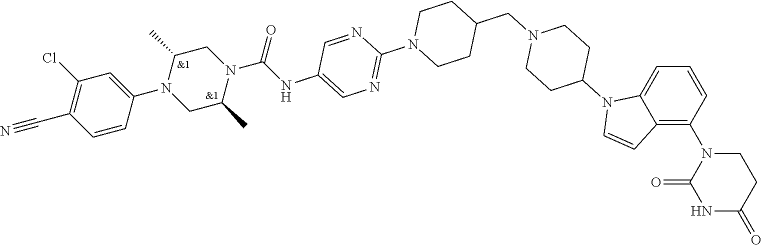 [SMILES: C[C@@H]1CN(C(=O)Nc2cnc(N3CCC(CN4CCC(n5ccc6c(N7CCC(=O)NC7=O)cccc65)CC4)CC3)nc2)[C@@H](C)CN1c1ccc(C#N)c(Cl)c1]

rac-(trans)-4-(3-Chloro-4-cyanophenyl)-N-(2-(4-formylpiperidin-1-yl)pyrimidin-5-yl)-2,5-dimethylpiperazine-1-carboxamide (may be prepared as described in Description 163; 150 mg, 0.311 mmol) and 1-(1-(piperidin-4-yl)-1H-indol-4-yl)dihydropyrimidine-2,4(1H,3H)-dione (may be prepared as described in Example CB1; 97 mg, 0.311 mmol) were suspended in DCM (20 mL) and stirred for 30 min at room temperature then sodium triacetoxyborohydride (198 mg, 0.934 mmol) and MeOH (0.5 mL) were added. The resulting solution was stirred overnight at room temperature. Fresh sodium triacetoxyborohydride (200 mg, 0.944 mmol) from a new bottle was added, then the mixture stirred for 2 h at room temperature. The mixture was washed with saturated sodium bicarbonate solution, the solvent dried and evaporated in vacuo to give a pale yellow gum. The crude material was dissolved in DCM and loaded onto a 24 g silica column, then eluted with 0-100% (25% ethanol/EtOAc 1% NH₄OH) and product-containing fractions were evaporated in vacuo to give the title compound (85 mg, 0.109 mmol, 35% yield) as a colourless solid. LCMS (high pH A): Rt=1.22 min, MH⁺=778.2, 780.2

### Example AR115

### rac-(trans)-4-(3-Chloro-4-cyanophenyl)-N-(6-(4-(2-(4-(4-(2,4-dioxotetrahydropyrimidin-1(2H)-yl)-1H-indol-1-yl)piperidin-1-yl)ethyl)piperidin-1-yl)pyridin-3-yl)-2,5-dimethylpiperazine-1-carboxamide (EAR115)

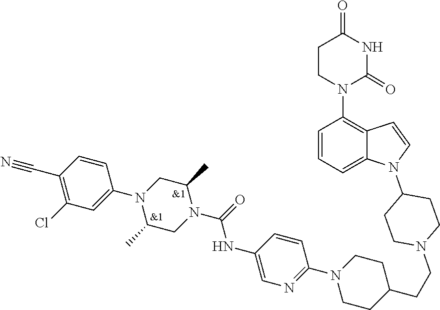 [SMILES: C[C@@H]1CN(c2ccc(C#N)c(Cl)c2)[C@@H](C)CN1C(=O)Nc1ccc(N2CCC(CCN3CCC(n4ccc5c(N6CCC(=O)NC6=O)cccc54)CC3)CC2)nc1]

rac-(trans)-4-(3-chloro-4-cyanophenyl)-2,5-dimethyl-N-(6-(4-(2-oxoethyl)piperidin-1-yl)pyridin-3-yl)piperazine-1-carboxamide (may be prepared as described in Description 170; 160 mg, 0.323 mmol) and 1-(1-(piperidin-4-yl)-1H-indol-4-yl)dihydropyrimidine-2,4(1H,3H)-dione (may be prepared as described in Example CB1; 101 mg, 0.323 mmol) were suspended in DCM (20 mL) and stirred for 30 min at room temperature then sodium triacetoxyborohydride (206 mg, 0.970 mmol) was added and the mixture stirred for 1 h at room temperature. The mixture was quenched with saturated sodium bicarbonate solution and stirred for 20 min, then the organic layer separated, dried and evaporated in vacuo and the residue purified by chromatography on a 24 g silica column eluting with 0-100% (25% ethanol/EtOAc 1% NH₄OH)/cyclohexane. Product-containing fractions were evaporated in vacuo to give the title compound as a yellow solid (172 mg, 0.217 mmol, 67% yield). LCMS (high pH A): Rt=1.22 min, MH⁺=791.5, 793.5

### Example AR116

### (2S,5R)-4-(3-Chloro-4-cyanophenyl)-N-(2-(4-((4-(4-(2,4-dioxotetrahydropyrimidin-1(2H)-yl)-1H-indol-1-yl)piperidin-1-yl)methyl)piperidin-1-yl)pyrimidin-5-yl)-2,5-dimethylpiperazine-1-carboxamide (EAR116)

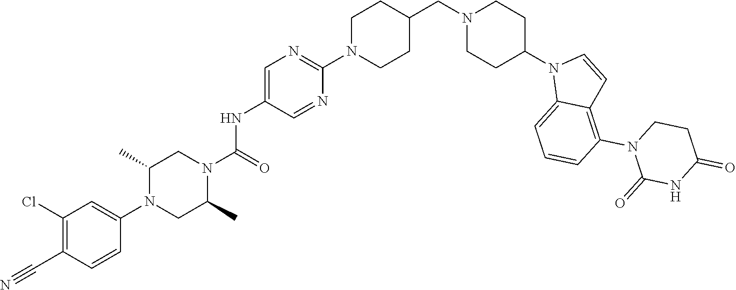 [SMILES: C[C@@H]1CN(C(=O)Nc2cnc(N3CCC(CN4CCC(n5ccc6c(N7CCC(=O)NC7=O)cccc65)CC4)CC3)nc2)[C@@H](C)CN1c1ccc(C#N)c(Cl)c1]

A mixture of 1-(1-(piperidin-4-yl)-1H-indol-4-yl)dihydropyrimidine-2,4(1H,3H)-dione (may be prepared as described in Example CB1; 123 mg, 0.394 mmol) and (2S,5R)-4-(3-chloro-4-cyanophenyl)-N-(2-(4-formylpiperidin-1-yl)pyrimidin-5-yl)-2,5-dimethylpiperazine-1-carboxamide (176 mg, 0.365 mmol) was stirred in DCM (5 mL) at room temperature for 30 min. Sodium triacetoxyborohydride (232 mg, 1.095 mmol) was added to the reaction mixture and stirred at room temperature for 17 h. The reaction mixture was partitioned between DCM (15 mL) and saturated sodium hydrogen carbonate (15 mL). The aqueous phase was separated. The organic phase was washed with water (10 mL) and brine (10 mL). The organic phase was dried and evaporated. The residue was purified by MDAP (formic) to give the title compound (86 mg, 0.110 mmol, 30% yield) as an off white solid. LCMS (high pH A): Rt=1.22 min, MH⁺=778.4, 780.4

### Examples AR117-AR118

### General Procedure

(2R,5S)-4-(3-Chloro-4-cyanophenyl)-2,5-dimethyl-N-(6-(4-(2-oxoethyl)piperidin-1-yl)pyridin-3-yl)piperazine-1-carboxamide (may be prepared as described in Description 170; 80 mg, 0.162 mmol) and the appropriate amine (0.162 mmol) was suspended in DCM (5 mL) and stirred for 30 min at room temperature. Sodium triacetoxyborohydride (68.5 mg, 0.323 mmol) was added and the mixture stirred for 1 h at room temperature. The reaction mixture was quenched with saturated sodium bicarbonate solution and stirred for 20 min. The phases were separated and the solutions blown down to give crude compounds as brown gums which were purified using the method specified.

| | | | Purification |
|---|---|---|---|
| | Name | Inter- | Method; |
| Example | Analytical | mediate | Mass; Yield |
| AR117 | 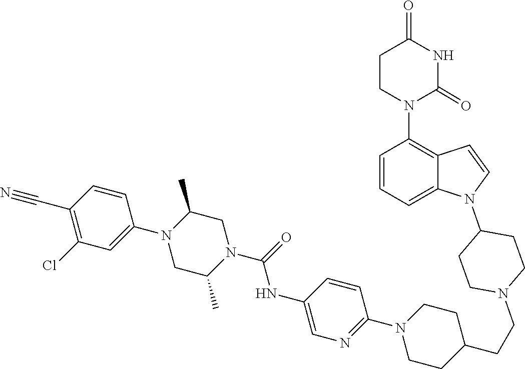 [SMILES: C[C@@H]1CN(c2ccc(C#N)c(Cl)c2)[C@@H](C)CN1C(=O)Nc1ccc(N2CCC(CCN3CCC(n4ccc5c(N6CCC(=O)NC6=O)cccc54)CC3)CC2)nc1] | ECB1 | MDAP (high pH) 33 mg, 26% |
| | (2R,5S)-4-(3-Chloro-4-cyanopheny)-N-(6-(4-(2-(4-(4- | | |
| | (2,4-dioxotetrahydropyrimidin-1(2H)-yl)-1H-indol-1- | | |
| | yl)piperidin-1-yl)ethyl)piperidin-1-yl)pyridin-3-yl)-2,5- | | |
| | dimethylpiperazine-1-carboxamide | | |
| | LCMS (high pH A): Rt = 1.21 min, MH⁺ = 791.3, 793.3 | | |
| AR118 | 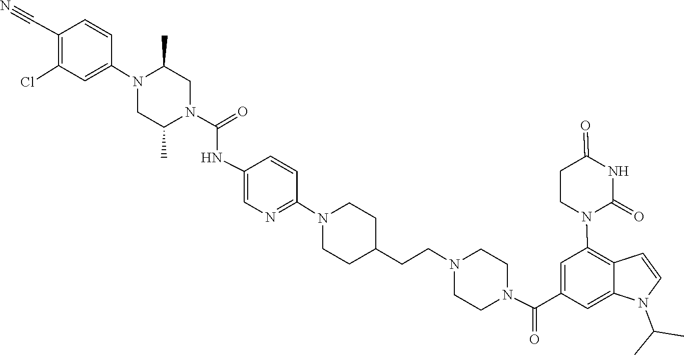 [SMILES: CC(C)n1ccc2c(N3CCC(=O)NC3=O)cc(C(=O)N3CCN(CCC4CCN(c5ccc(NC(=O)N6C[C@H](C)N(c7ccc(C#N)c(Cl)c7)C[C@H]6C)cn5)CC4)CC3)cc21] | ECB11 | MDAP (high pH) 41 mg, 29% |
| | (2R,5S)-4-(3-Chloro-4-cyanophenyl)-N-(6-(4-(2-(4-(4- | | |
| | (2,4-dioxotetrahydropyrimidin-1(2H)-yl)-1-isopropyl-1H- | | |
| | indole-6-carbonyl)piperazin-1-yl)ethyl)piperidin-1- | | |
| | yl)pyridin-3-yl)-2,5-dimethylpiperazine-1-carboxamide | | |
| | LCMS (high pH A): Rt = 1.14 min, MH⁺ = 862.4, 864.4 | | |

### Example AR119

### N-((1r,4r)-4-(3-Chloro-4-cyanophenoxy)cyclohexyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide (EAR119)

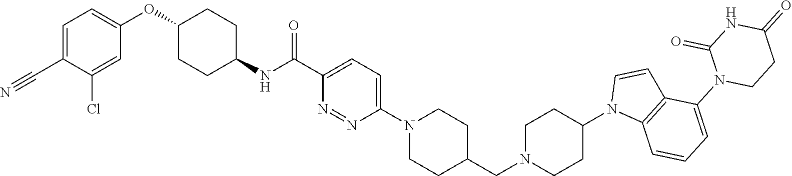 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)nn3)CC2)cc1Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 71.5 mg, 0.153 mmol), 1-(7-(piperidin-4-yl)-7H-pyrrolo[2,3-d]pyrimidin-4-yl)dihydropyrimidine-2,4(1H,3H)-dione (may be prepared as described in Example CB17; 40 mg, 0.127 mmol) DCM (2 mL) were mixed and stirred at room temperature for 10 min. Sodium triacetoxyborohydride (81 mg, 0.382 mmol) was added and the reaction mixture stirred at room temperature overnight. The reaction mixture was diluted with DCM. NaHCO₃ and brine were added and the mixture shaken vigorously, passed through a hydrophobic frit and blown down. The material was purified by MDAP (high pH) and the fractions evaporated. Drying in a vacuum oven at 40° C. overnight (approximately 16 h) gave the title compound as a slightly off-white powder (40 mg, 0.050 mmol, 39% yield). LCMS (high pH A): Rt=1.21 min, MH⁺=766. ¹H NMR (400 MHZ, DMSO-d₆) δ (ppm) 10.69 (s, 1H), 8.64 (s, 1H), 8.55 (d, J=8.1 Hz, 1H), 7.86 (d, J=8.8 Hz, 1H), 7.81 (d, J=9.8 Hz, 1H), 7.68 (d, J=3.7 Hz, 1H), 7.38 (d, J=2.4 Hz, 1H), 7.34 (d, J=9.8 Hz, 1H), 7.14 (dd, J=8.8, 2.4 Hz, 1H), 6.42 (d, J=3.7 Hz, 1H), 4.59-4.73 (m, 1H), 4.43-4.59 (m, 3H), 4.08 (t, J=6.5 Hz, 2H), 3.81-3.93 (m, 1H), 2.97-3.10 (m, 4H), 2.74 (t, J=6.5 Hz, 2H), 2.20-2.30 (m, 2H), 2.04-2.19 (m, 6H), 1.81-1.97 (m, 7H), 1.42-1.75 (m, 4H), 1.04-1.24 (m, 2H)

### Example AR120

### N-((1r,4r)-4-(3-Chloro-4-cyanophenoxy)cyclohexyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide hydrochloride (EAR120)

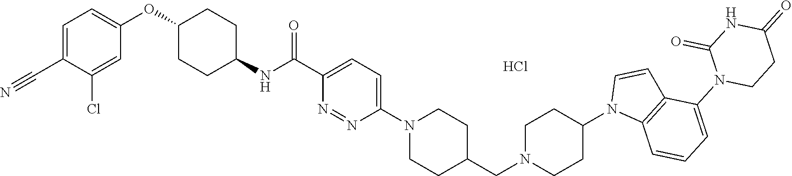 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)nn3)CC2)cc1Cl ; Cl]

A mixture of N-((1r,4r)-4-(3-chloro-4-cyanophenoxy)cyclohexyl)-6-(4-formylpiperidin-1-yl)pyridazine-3-carboxamide ((may be prepared as described in Description 95; 182 mg, 0.389 mmol) and 1-(7-(piperidin-4-yl)-7H-pyrrolo[2,3-d]pyrimidin-4-yl)dihydropyrimidine-2,4(1H,3H)-dione (may be prepared as described in Example CB17; 102 mg, 0.324 mmol) in DCM (10 mL) were mixed and stirred at room temperature for 10 min. Sodium triacetoxyborohydride (206 mg, 0.973 mmol) was added and the reaction mixture stirred at room temperature for 15 h. The reaction mixture was diluted with NaHCO₃ (10 mL) and brine (5 mL) and the layers were separated. The aqueous layer was extracted with DCM (4 mL) and the combined organic layers were passed through a hydrophobic frit and concentrated using a stream of nitrogen gas. The resulting colourless gum was dissolved in DMSO (2 mL) and purified using a C18 XBridge OBD column, eluting with 30-90% MeCN in 10 mM ammonium carbonate solution. The desired fractions were combined and the MeCN was removed in vacuo. To the resulting mixture was added DCM (20 mL) and sat. aqueous sodium hydrogen carbonate (4 mL), and the layers were separated. The aqueous layer was extracted with DCM (2×6 mL), and the combined organic layers were passed through a hydrophobic frit and concentrated in vacuo. The residue was dissolved in DCM (2 mL) and to the pale yellow coloured solution was added 4 M HCl in in 1,4-dioxane (77 μL). A white solid precipitated and the mixture was stirred for 30 min followed by removal of the stirrer bar and removal of the solvent under a stream of nitrogen gas. The resulting white solid was dried in a vacuum oven for 16 h to give the title compound as a white coloured solid (167 mg, 0.208 mmol, 64% yield). LCMS (high pH A): Rt=1.20 min, MH⁺=766. ¹H NMR (600 MHZ, DMSO-d₆): δ (ppm) 10.73 (s, 1H), 10.24-10.34 (m, 1H), 8.67 (s, 1H), 8.59 (d, J=8.1 Hz, 1H), 7.86 (d, J=8.8 Hz, 1H), 7.83 (d, J=9.6 Hz 1H), 7.51 (d, J=3.6 Hz, 1H), 7.42 (d, J=9.6 Hz, 1H), 7.39 (d, J=2.3 Hz, 1H), 7.13 (dd, J=8.8, 2.4 Hz, 1H), 6.50 (d, J=3.6 Hz, 1H), 5.00-5.07 (m, 1H), 4.51-4.56 (m, 1H), 4.49-4.54 (m, 2H), 4.09 (t, J=6.5 Hz, 2H), 3.83-3.90 (m, 1H), 3.66-3.72 (m, 2H), 3.21-3.30 (m, 2H), 3.05-3.11 (m, 2H), 3.02-3.06 (m, 2H), 2.74 (t, J=6.5 Hz, 2H), 2.65-2.72 (m, 2H), 2.22-2.30 (m, 1H), 2.13-2.18 (m, 2H), 2.08-2.13 (m, 2H), 1.96-2.03 (m, 2H), 1.87-1.93 (m, 2H), 1.60-1.68 (m, 2H), 1.48-1.56 (m, 2H), 1.25-1.32 (m, 2H).

### Example AR121

### (2S,5R)-4-(3-chloro-4-cyanophenyl)-N-(6-(4-(4-(4-(2,4-dioxotetrahydropyrimidin-1(2H)-yl)-1H-indol-1-yl)piperidin-1-yl)butoxy)pyridin-3-yl)-2,5-dimethylpiperazine-1-carboxamide (EAR121)

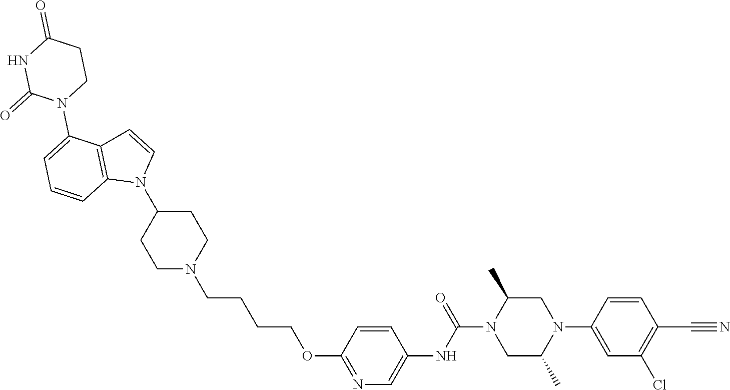 [SMILES: C[C@@H]1CN(C(=O)Nc2ccc(OCCCCN3CCC(n4ccc5c(N6CCC(=O)NC6=O)cccc54)CC3)nc2)[C@@H](C)CN1c1ccc(C#N)c(Cl)c1]

A mixture of 1-(1-(piperidin-4-yl)-1H-indol-4-yl)dihydropyrimidine-2,4(1H,3H)-dione (may be prepared as described in Example ECB1; 234 mg, 0.749 mmol) and (2S,5R)-4-(3-chloro-4-cyanophenyl)-N-(2-(4-formylpiperidin-1-yl)pyrimidin-5-yl)-2,5-dimethylpiperazine-1-carboxamide (may be prepared as described in Description 164; 318 mg, 0.697 mmol) was stirred in DCM (6 mL) at room temperature for 30 minutes. Sodium triacetoxyborohydride (451 mg, 2.128 mmol) was added, and the reaction mixture was stirred at room temperature for 4 h. The reaction mixture was partitioned between DCM (25 mL) and saturated sodium hydrogen carbonate (25 mL). The organic phase was separated, and the aqueous phase was extracted with DCM (10 mL). The combined organic phases were washed with water (10 mL) and brine (10 mL), dried and evaporated. The residue was purified by formic acid MDAP to give (2S,5R)-4-(3-chloro-4-cyanophenyl)-N-(6-(4-(4-(4-(2,4-dioxotetrahydropyrimidin-1(2H)-yl)-1H-indol-1-yl)piperidin-1-yl)butoxy)pyridin-3-yl)-2,5-dimethylpiperazine-1-carboxamide (63 mg, 0.084 mmol, 12.01% yield). LCMS (formic A): Rt=0.78 min, MH⁺=752, 754.

### Example AR122

### N-((1r,4r)-4-(3-chloro-4-cyanophenoxy)cyclohexyl)-6-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)pyridazine-3-carboxamide (EAR122)

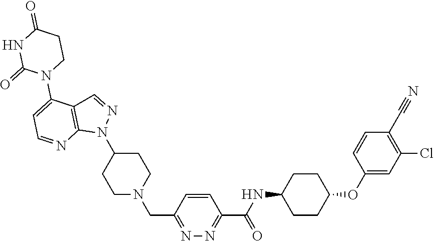 [SMILES: N#Cc1ccc(O[C@H]2CC[C@H](NC(=O)c3ccc(CN4CCC(n5ncc6c(N7CCC(=O)NC7=O)ccnc65)CC4)nn3)CC2)cc1Cl]

To N-((1r,4r)-4-(3-chloro-4-cyanophenoxy)cyclohexyl)-6-(4-formylpiperidin-1-yl)pyridazine-3-carboxamide (may be prepared as described in Description 95; 71.5 mg, 0.153 mmol) and 1-(1-(piperidin-4-yl)-1H-pyrazolo[3,4-b]pyridin-4-yl)dihydropyrimidine-2,4(1H,3H)-dione (may be prepared as described in Example ECB19; 40 mg, 0.127 mmol) in DCM (2 mL) was added sodium triacetoxyborohydride (81 mg, 0.382 mmol). After stirring overnight, the reaction was diluted with DCM, and NaHCO₃ and brine were added. The mixture was shaken vigorously, passed through a hydrophobic frit and blown down. Purification by MDAP (high pH) gave N-((1r,4r)-4-(3-chloro-4-cyanophenoxy)cyclohexyl)-6-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)pyridazine-3-carboxamide (40 mg, 0.050 mmol, 39.0% yield). LCMS (high pH A): Rt=1.19 min, MH⁺=766.5, 768.5.

### Example AR123

### N-((1r,4R)-4-(3-Chloro-4-cyanophenoxy)cyclohexyl)-6-((R)-2-((4-(4-(2,4-dioxotetrahydropyrimidin-1(2H)-yl)-1H-indol-1-yl)piperidin-1-yl)methyl)morpholino)pyridazine-3-carboxamide (EAR123)

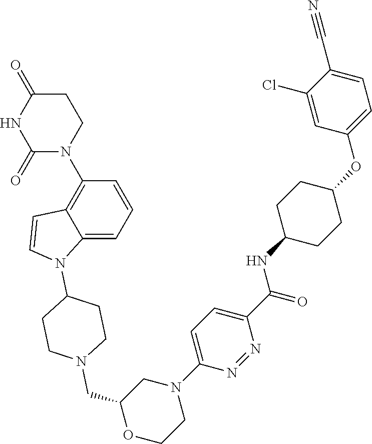 [SMILES: N#Cc1ccc(O[C@H]2CC[C@H](NC(=O)c3ccc(N4CCO[C@H](CN5CCC(n6ccc7c(N8CCC(=O)NC8=O)cccc76)CC5)C4)nn3)CC2)cc1Cl]

To 1-(1-(piperidin-4-yl)-1H-indol-4-yl)dihydropyrimidine-2,4(1H,3H)-dione (may be prepared as described in Example ECB1; 40 mg, 0.128 mmol) and N-((1r,4S)-4-(3-chloro-4-cyanophenoxy)cyclohexyl)-6-((S)-2-formylmorpholino)pyridazine-3-carboxamide (may be prepared as described in Description 191; 50 mg, 0.106 mmol) suspended in DCM (20 ml) at room temperature was added sodium triacetoxyborohydride (54.3 mg, 0.256 mmol). After 1 h, more sodium triacetoxyborohydride (54.3 mg, 0.256 mmol) was added, and the mixture stirred for 2 h. Sodium bicarbonate solution (20 ml) was added and the mixture stirred for 20 min, the phases were separated, and the organic layer was dried and evaporated in vacuo to give a pale yellow gum which was purified by MDAP (high pH) to give N-((1r,4R)-4-(3-chloro-4-cyanophenoxy)cyclohexyl)-6-((R)-2-((4-(4-(2,4-dioxotetrahydropyrimidin-1(2H)-yl)-1H-indol-1-yl)piperidin-1-yl)methyl)morpholino)pyridazine-3-carboxamide (41.9 mg, 0.055 mmol, 42.7% yield). LCMS (high pH A): Rt=1.18 min, MH⁺=766.

### Example AR124

### N-((1r,4S)-4-(3-Chloro-4-cyanophenoxy)cyclohexyl)-6-((S)-2-((4-(4-(2,4-dioxotetrahydropyrimidin-1(2H)-yl)-1H-indazol-1-yl)piperidin-1-yl)methyl)morpholino)pyridazine-3-carboxamide (EAR124)

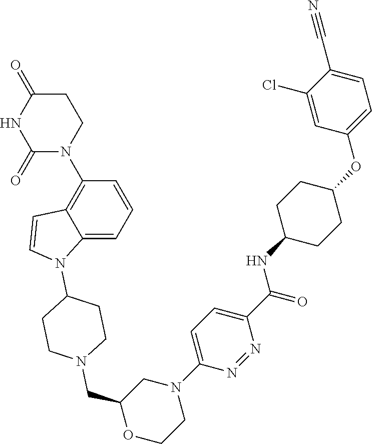 [SMILES: N#Cc1ccc(O[C@H]2CC[C@H](NC(=O)c3ccc(N4CCO[C@@H](CN5CCC(n6ccc7c(N8CCC(=O)NC8=O)cccc76)CC5)C4)nn3)CC2)cc1Cl]

To 1-(1-(Piperidin-4-yl)-1H-indazol-4-yl)dihydropyrimidine-2,4(1H,3H)-dione, trifluoroacetic acid salt (may be prepared as described in Example ECB4; 50 mg, 0.117 mmol) and N-((1r,4R)-4-(3-chloro-4-cyanophenoxy)cyclohexyl)-6-((R)-2-formylmorpholino)pyridazine-3-carboxamide (may be prepared as described in Description 193; 50 mg, 0.117 mmol) suspended in DCM (20 ml) at room temperature was added DIPEA (0.020 mL, 0.117 mmol), and the mixture was stirred for 1 h. Sodium triacetoxyborohydride (49.6 mg, 0.234 mmol) was added, and the mixture was stirred for 2 h. Sodium bicarbonate solution (20 ml) was added, the mixture was stirred for 20 min, the phases were separated and the organic layer was dried and evaporated in vacuo to give a pale yellow gum which was purified by MDAP (high pH) to give N-((1r,4S)-4-(3-chloro-4-cyanophenoxy)cyclohexyl)-6-((S)-2-((4-(4-(2,4-dioxotetrahydropyrimidin-1(2H)-yl)-1H-indazol-1-yl)piperidin-1-yl)methyl)morpholino)pyridazine-3-carboxamide (18.5 mg, 0.024 mmol, 20.61% yield). LCMS (high pH A): Rt=1.14 min, MH⁺=767.

### Example AR125

### N-((1r,4S)-4-(3-Chloro-4-cyanophenoxy)cyclohexyl)-6-((S)-2-((4-(4-(2,4-dioxotetrahydropyrimidin-1(2H)-yl)-1H-indol-1-yl)piperidin-1-yl)methyl)morpholino)pyridazine-3-carboxamide (EAR125)

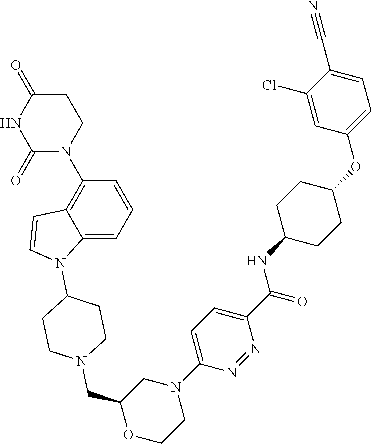 [SMILES: N#Cc1ccc(O[C@H]2CC[C@H](NC(=O)c3ccc(N4CCO[C@@H](CN5CCC(n6ccc7c(N8CCC(=O)NC8=O)cccc76)CC5)C4)nn3)CC2)cc1Cl]

To 1-(1-(piperidin-4-yl)-1H-indol-4-yl)dihydropyrimidine-2,4(1H,3H)-dione (may be prepared as described in Example ECB1; 40 mg, 0.128 mmol) and N-((1r,4R)-4-(3-chloro-4-cyanophenoxy)cyclohexyl)-6-((R)-2-formylmorpholino)pyridazine-3-carboxamide (may be prepared as described in Description 193; 50 mg, 0.117 mmol) suspended in DCM (20 ml) at room temperature was added sodium triacetoxyborohydride (54.3 mg, 0.256 mmol), and the mixture was stirred overnight. Sodium bicarbonate solution (20 ml) was added, the mixture was stirred for 20 min, the phases were separated and the organic layer was dried and evaporated in vacuo to give a pale yellow gum which was purified by MDAP (high pH) to give N-((1r,4S)-4-(3-chloro-4-cyanophenoxy)cyclohexyl)-6-((S)-2-((4-(4-(2,4-dioxotetrahydropyrimidin-1(2H)-yl)-1H-indol-1-yl)piperidin-1-yl)methyl)morpholino)pyridazine-3-carboxamide (27.7 mg, 0.036 mmol, 28.2% yield). LCMS (high pH A): Rt=1.18 min, MH⁺=766.

### Example AR127

### N-((1r,4R)-4-(3-Chloro-4-cyanophenoxy)cyclohexyl)-6-((R)-2-((4-(4-(2,4-dioxotetrahydropyrimidin-1(2H)-yl)-1H-indazol-1-yl)piperidin-1-yl)methyl)morpholino)pyridazine-3-carboxamide (EAR127)

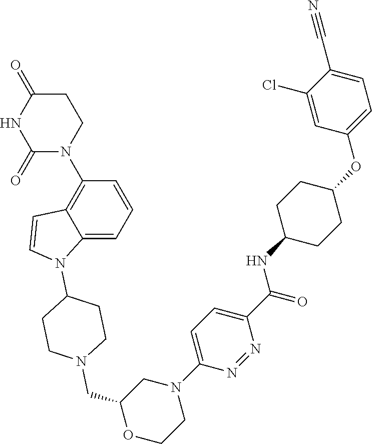 [SMILES: N#Cc1ccc(O[C@H]2CC[C@H](NC(=O)c3ccc(N4CCO[C@H](CN5CCC(n6ccc7c(N8CCC(=O)NC8=O)cccc76)CC5)C4)nn3)CC2)cc1Cl]

To 1-(1-(piperidin-4-yl)-1H-indazol-4-yl)dihydropyrimidine-2,4(1H,3H)-dione trifluoroacetic acid salt (may be prepared as described in Example ECB4; 50 mg, 0.117 mmol) and N-((1r,4S)-4-(3-chloro-4-cyanophenoxy)cyclohexyl)-6-((S)-2-formylmorpholino)pyridazine-3-carboxamide (may be prepared as described in Description 191; 50 mg, 0.106 mmol) suspended in DCM (20 ml) at room temperature was added DIPEA (0.020 mL, 0.117 mmol). After 1 h sodium triacetoxyborohydride (49.6 mg, 0.234 mmol) was added, and the mixture was stirred for 2 h. Sodium bicarbonate solution (20 ml) was added, and the mixture was stirred for 20 min. The phases were separated, and the organic layer was dried and evaporated in vacuo to give a pale yellow gum which was purified by MDAP (high pH) to give N-((1r,4R)-4-(3-chloro-4-cyanophenoxy)cyclohexyl)-6-((R)-2-((4-(4-(2,4-dioxotetrahydropyrimidin-1(2H)-yl)-1H-indazol-1-yl)piperidin-1-yl)methyl)morpholino)pyridazine-3-carboxamide (37.1 mg, 0.048 mmol, 41.3% yield). LCMS (high pH A): Rt=1.14 min, MH⁺=767.2

### Example AR128

### N-((1r,4r)-4-(3-Chloro-4-cyanophenoxy)cyclohexyl)-6-(4-((4-(4-(2,4-dioxotetrahydropyrimidin-1(2H)-yl)-1H-indazol-1-yl)piperidin-1-yl)methyl)-4-fluoropiperidin-1-yl)pyridazine-3-carboxamide (EAR128)

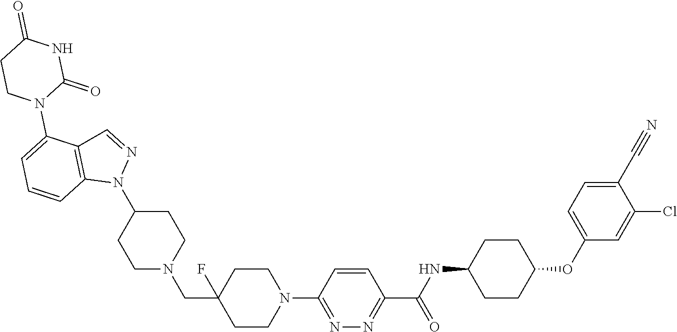 [SMILES: N#Cc1ccc(O[C@H]2CC[C@H](NC(=O)c3ccc(N4CCC(F)(CN5CCC(n6ncc7c(N8CCC(=O)NC8=O)cccc76)CC5)CC4)nn3)CC2)cc1Cl]

A mixture of 1-(1-(1-((4-fluoropiperidin-4-yl)methyl)piperidin-4-yl)-1H-indazol-4-yl)dihydropyrimidine-2,4(1H,3H)-dione hydrochloride (may be prepared as described in Example ECBL6; 85 mg, 0.183 mmol), 6-chloro-N-((1r,4r)-4-(3-chloro-4-cyanophenoxy)cyclohexyl)pyridazine-3-carboxamide (may be prepared as described in Description 90; 48 mg, 0.123 mmol) and diisopropylethylamine (48 mg, 0.065 mL, 0.371 mmol) in N-methyl-2-pyrrolidone (2 mL) was heated at 110° C. for 16 hours. Potassium carbonate (60 mg, 0.434 mmol) was added, and the reaction mixture was stirred at 120° C. for 30 hours. The reaction was cooled to room temperature and partitioned between ethyl acetate (10 mL) and water (15 mL). The organic phase was separated. The aqueous phase was extracted with ethyl acetate (2×10 mL). The combined organics were washed with brine (10 mL), dried and evaporated. The residue was chromatographed [50-100% ethyl acetate in cyclohexane, 0-30% ethanol in ethyl acetate] to give partially pure material which was repurified by MDAP (formic) to give N-((1r,4r)-4-(3-chloro-4-cyanophenoxy)cyclohexyl)-6-(4-((4-(4-(2,4-dioxotetrahydropyrimidin-1(2H)-yl)-1H-indazol-1-yl)piperidin-1-yl)methyl)-4-fluoropiperidin-1-yl)pyridazine-3-carboxamide (13 mg, 0.017 mmol, 13.53% yield). LCMS (formic A): Rt=0.81 min, MH⁺=783.4

### Example AR129

### N-((1r,4r)-4-(3-Chloro-4-cyanophenoxy)cyclohexyl)-6-(4-((4-(4-(2,4-dioxotetrahydropyrimidin-1(2H)-yl)-1H-pyrazolo[3,4-c]pyridin-1-yl)piperidin-1-yl)methyl)-4-fluoropiperidin-1-yl)pyridazine-3-carboxamide (EAR129)

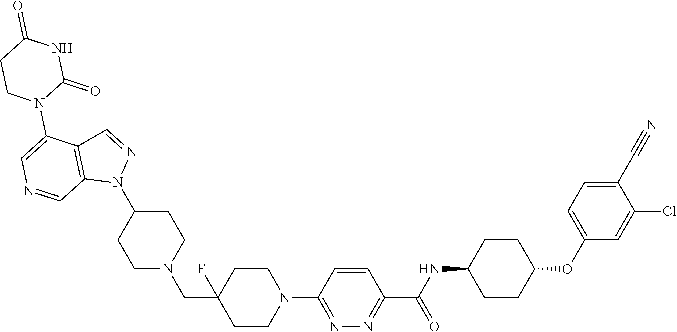 [SMILES: N#Cc1ccc(O[C@H]2CC[C@H](NC(=O)c3ccc(N4CCC(F)(CN5CCC(n6ncc7c(N8CCC(=O)NC8=O)cncc76)CC5)CC4)nn3)CC2)cc1Cl]

A mixture of 1-(1-(1-((4-fluoropiperidin-4-yl)methyl)piperidin-4-yl)-1H-pyrazolo[3,4-c]pyridin-4-yl)dihydropyrimidine-2,4(1H,3H)-dione hydrochloride (may be prepared as described in Example ECBL7; 90 mg, 0.194 mmol), 6-chloro-N-((1r,4r)-4-(3-chloro-4-cyanophenoxy)cyclohexyl)pyridazine-3-carboxamide (may be prepared as described in Description 90; 50 mg, 0.129 mmol) and diisopropylethylamine (50 mg, 0.068 mL, 0.387 mmol) in N-methyl-2-pyrrolidone (2 mL) was heated at 110° C. for 16 hours. Potassium carbonate (60 mg, 0.434 mmol) was added, and the reaction mixture was stirred at 120° C. for 30 hours. The reaction was cooled to room temperature and partitioned between ethyl acetate (10 mL) and water (15 mL). The organic phase was separated. The aqueous phase was extracted with ethyl acetate (2×10 mL). The combined organics were washed with brine (10 mL), dried and evaporated. The residue was chromatographed [50-100% ethyl acetate in cyclohexane, 0-30% ethanol in ethyl acetate] to give partially pure material which was repurified by MDAP (formic) to give N-((1r,4r)-4-(3-chloro-4-cyanophenoxy)cyclohexyl)-6-(4-((4-(4-(2,4-dioxotetrahydropyrimidin-1(2H)-yl)-1H-pyrazolo[3,4-c]pyridin-1-yl)piperidin-1-yl)methyl)-4-fluoropiperidin-1-yl)pyridazine-3-carboxamide (38 mg, 0.048 mmol, 37.6% yield). LCMS (formic A): Rt=0.78 min, MH⁺=782.4

### Example AR130

### N-((1r,4r)-4-(3-Chloro-4-cyanophenoxy)cyclohexyl)-4-(2-(4-(4-(2,4-dioxotetrahydropyrimidin-1(2H)-yl)-1H-indol-1-yl)piperidin-1-yl)ethyl)benzamide (EAR130)

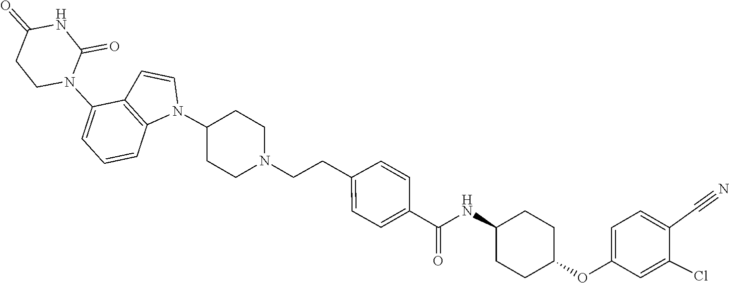 [SMILES: N#Cc1ccc(O[C@H]2CC[C@H](NC(=O)c3ccc(CCN4CCC(n5ccc6c(N7CCC(=O)NC7=O)cccc65)CC4)cc3)CC2)cc1Cl]

To a stirring solution of 4-(((1r,4r)-4-(3-chloro-4-cyanophenoxy)cyclohexyl)carbamoyl)phenethyl 4-methylbenzenesulfonate (may be prepared as described in Description 195; 135 mg, 0.244 mmol) and sodium bicarbonate (61.5 mg, 0.732 mmol) in N,N-Dimethylformamide (DMF) (0.8 mL) was added 1-(1-(piperidin-4-yl)-1H-indol-4-yl)dihydropyrimidine-2,4(1H,3H)-dione (may be prepared as described in Example CB1; 76 mg, 0.244 mmol), followed by stirring whist heating to 60° C. for 24 h. The solution was cooled to rt, diluted with EtOAc (10 mL) and water (10 mL), and the layers were separated. The aqueous layer was extracted with DCM (2×5 mL), and the combined organic layers were concentrated in vacuo to give a yellow solid which was purified by MDAP (formic) to give N-((1r,4r)-4-(3-chloro-4-cyanophenoxy)cyclohexyl)-4-(2-(4-(4-(2,4-dioxotetrahydropyrimidin-1(2H)-yl)-1H-indol-1-yl)piperidin-1-yl)ethyl)benzamide (11.9 mg, 0.017 mmol, 7.03% yield). LCMS (formic A): Rt=1.21 min, MH⁺=693.2

### Example AR131

### N-((1r,4r)-4-(3-chloro-4-cyanophenoxy)cyclohexyl)-6-(4-((3-(4-(2,4-dioxotetrahydropyrimidin-1(2H)-yl)-1H-indol-1-yl) azetidin-1-yl)methyl)piperidin-1-yl)pyridazine-3-carboxamide (EAR131)

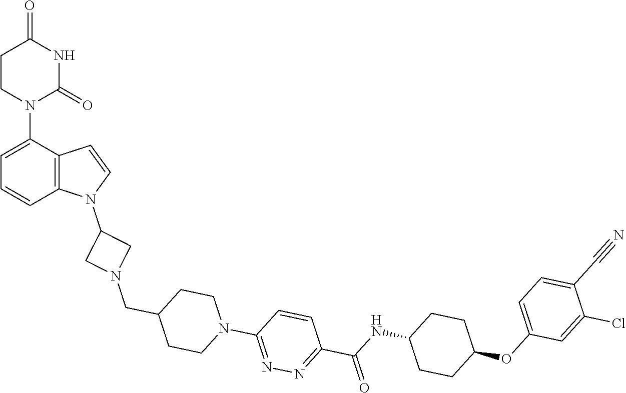 [SMILES: N#Cc1ccc(O[C@H]2CC[C@H](NC(=O)c3ccc(N4CCC(CN5CC(n6ccc7c(N8CCC(=O)NC8=O)cccc76)C5)CC4)nn3)CC2)cc1Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 104 mg, 0.222 mmol) and 1-(1-(azetidin-3-yl)-1H-indol-4-yl)dihydropyrimidine-2,4(1H,3H)-dione (may be described as prepared in Example ECB20; 52.7 mg, 0.185 mmol) were suspended in Dichloromethane (DCM) (2.6 mL), and the mixture stirred at room temperature under nitrogen for 10 min. Sodium triacetoxyborohydride (118 mg, 0.556 mmol) was added, and the reaction mixture stirred at room temperature for 17 hr. The reaction mixture was diluted with DCM (20 mL), NaHCO₃ (15 mL) and brine (5 mL) and shaken vigorously. The layers were separated and the aqueous extracted with DCM (10 mL). The organics were combined, dried by passing through a hydrophobic frit, and the solvent removed in vacuo to give the crude material which was dissolved in DMSO:methanol 1:1 (1.5 mL) and purified by MDAP (high pH). The resulting white solids were dissolved in minimal DCM, transferred to a weighed vial and the solvent removed under a stream of nitrogen in a blowdown unit to give a white solid. To a solution of this material in 1,4-Dioxane (400 μL) stirred at room temperature was added HCl (4 M in dioxane) (0.060 mL, 0.238 mmol). The resulting suspension was stirred at room temperature for 2 h. The solvent was removed under a stream of nitrogen then further dried in a vacuum oven. This material was dissolved in DMSO:methanol 3:1 (1 mL) and purified by MDAP (high pH) to give N-((1r,4r)-4-(3-chloro-4-cyanophenoxy)cyclohexyl)-6-(4-((3-(4-(2,4-dioxotetrahydropyrimidin-1(2H)-yl)-1H-indol-1-yl) azetidin-1-yl)methyl)piperidin-1-yl)pyridazine-3-carboxamide (21.8 mg, 0.030 mmol, 15.97% yield). LCMS (high pH A): Rt=1.19 min, MH⁺=736.5, 738.4

### Example AR132

### (2S,5R)-4-(3-Chloro-4-cyanophenyl)-N-(6-(4-(2-(4-(4-(2,4-dioxotetrahydropyrimidin-1(2H)-yl)-1H-indazol-1-yl)piperidin-1-yl)ethyl)piperidin-1-yl)pyridin-3-yl)-2,5-dimethylpiperazine-1-carboxamide, formic acid salt (EAR132)

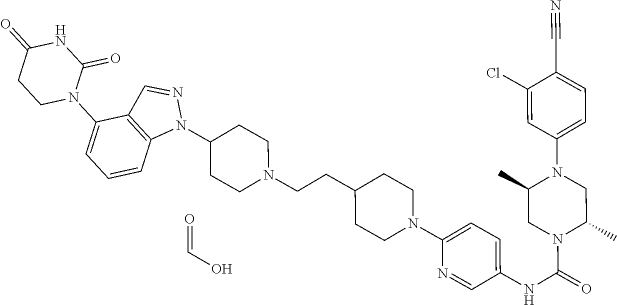 [SMILES: C[C@@H]1CN(C(=O)Nc2ccc(N3CCC(CCN4CCC(n5ncc6c(N7CCC(=O)NC7=O)cccc65)CC4)CC3)nc2)[C@@H](C)CN1c1ccc(C#N)c(Cl)c1 ; O=CO]

A mixture of 1-(1-(piperidin-4-yl)-1H-indazol-4-yl)dihydropyrimidine-2,4(1H,3H)-dione, trifluoroacetic acid salt (may be prepared as described in Example ECB4; 41 mg, 0.096 mmol) and (2S,5R)-4-(3-chloro-4-cyanophenyl)-2,5-dimethyl-N-(6-(4-(2-oxoethyl)piperidin-1-yl)pyridin-3-yl)piperazine-1-carboxamide (may be prepared as described in Description 15; 68 mg, 0.137 mmol) was stirred in DCM (6 mL) at room temperature for 0.75 h. Sodium triacetoxyborohydride (86 mg, 0.406 mmol) was added, and the mixture was stirred at room temperature for 2.5 h. The reaction was partitioned between DCM (25 mL) and saturated sodium hydrogen carbonate (25 mL). The organic phase was separated. The aqueous phase was extracted with DCM (10 mL×2). The combined organic phases were washed with water (30 mL) and brine (30 mL), then dried and evaporated. The residue was purified by MDAP (formic) to give (2S,5R)-4-(3-chloro-4-cyanophenyl)-N-(6-(4-(2-(4-(4-(2,4-dioxotetrahydropyrimidin-1(2H)-yl)-1H-indazol-1-yl)piperidin-1-yl)ethyl)piperidin-1-yl)pyridin-3-yl)-2,5-dimethylpiperazine-1-carboxamide, formic acid salt (22 mg, 0.026 mmol, 27.4% yield). LCMS (high pH A): Rt=1.17 min, MH⁺=790.4, 792.5

### Example AR133

### (2S,5R)-4-(3-Chloro-4-cyanophenyl)-N-(6-(4-(2-(4-(4-(2,4-dioxotetrahydropyrimidin-1(2H)-yl)-1H-pyrazolo[3,4-c]pyridin-1-yl)piperidin-1-yl)ethyl)piperidin-1-yl)pyridin-3-yl)-2,5-dimethylpiperazine-1-carboxamide, formic acid salt (EAR133)

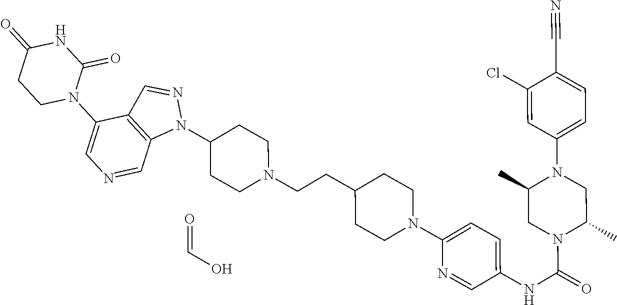 [SMILES: C[C@@H]1CN(C(=O)Nc2ccc(N3CCC(CCN4CCC(n5ncc6c(N7CCC(=O)NC7=O)cncc65)CC4)CC3)nc2)[C@@H](C)CN1c1ccc(C#N)c(Cl)c1 ; O=CO]

A mixture of 1-(1-(piperidin-4-yl)-1H-pyrazolo[3,4-c]pyridin-4-yl)dihydropyrimidine-2,4(1H,3H)-dione (may be prepared as described in Example ECB8; 46 mg, 0.146 mmol) and (2S,5R)-4-(3-chloro-4-cyanophenyl)-2,5-dimethyl-N-(6-(4-(2-oxoethyl)piperidin-1-yl)pyridin-3-yl)piperazine-1-carboxamide (may be prepared as described in Description D15; 95 mg, 0.192 mmol) was stirred in DCM (6 mL) at room temperature for 0.75 h. Sodium triacetoxyborohydride (451 mg, 2.128 mmol) was added to the mixture and stirred at room temperature for 2.5 h. The reaction was partitioned between DCM (25 mL) and saturated sodium hydrogen carbonate (25 mL). The organic phase was separated. The aqueous phase was extracted with DCM (10 mL×2). The combined organic phases were washed with water (30 mL) and brine (30 mL), and dried and evaporated. The residue was purified by MDAP (formic) to give (2S,5R)-4-(3-chloro-4-cyanophenyl)-N-(6-(4-(2-(4-(4-(2,4-dioxotetrahydropyrimidin-1(2H)-yl)-1H-pyrazolo[3,4-c]pyridin-1-yl)piperidin-1-yl)ethyl)piperidin-1-yl)pyridin-3-yl)-2,5-dimethylpiperazine-1-carboxamide, formic acid salt (28 mg, 0.033 mmol, 22.80% yield). LCMS (high pH A): Rt=1.08 min, MH⁺=791.4, 793.5

### Example AR134

### N-((1r,4r)-4-(3-Chloro-4-cyanophenoxy)cyclohexyl)-6-(4-(2-(4-(4-(2,4-dioxotetrahydropyrimidin-1(2H)-yl)-1H-indol-1-yl)piperidin-1-yl)-2-oxoethyl)piperazin-1-yl)pyridazine-3-carboxamide (EAR134)

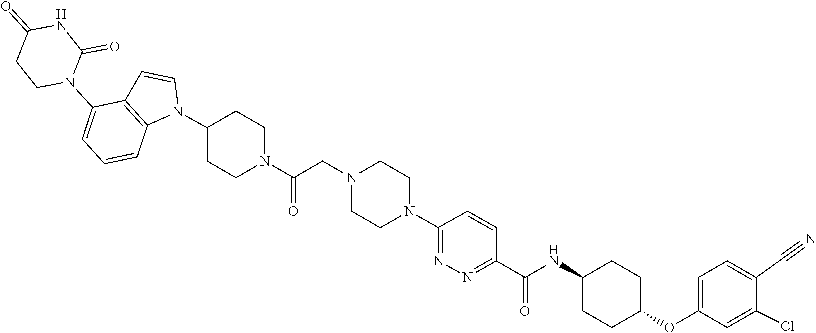 [SMILES: N#Cc1ccc(O[C@H]2CC[C@H](NC(=O)c3ccc(N4CCN(CC(=O)N5CCC(n6ccc7c(N8CCC(=O)NC8=O)cccc76)CC5)CC4)nn3)CC2)cc1Cl]

To a stirring solution of 2-(4-(6-(((1r,4r)-4-(3-chloro-4-cyanophenoxy)cyclohexyl)carbamoyl)pyridazin-3-yl)piperazin-1-yl) acetic acid (may be prepared as described in Description 204; 218 mg, 0.437 mmol) and 1-(1-(piperidin-4-yl)-1H-indol-4-yl)dihydropyrimidine-2,4(1H,3H)-dione (may be prepared as described in Example ECB1; 96 mg, 0.306 mmol; used substoichiometrically as the starting acid was approx 50% pure) and HATU (199 mg, 0.524 mmol) in N,N-Dimethylformamide (DMF) (3 mL) was added DIPEA (0.229 mL, 1.311 mmol), followed by stirring for 2 h. The solvent was partially removed using a flow of nitrogen gas until 1 mL of yellow coloured solution remained, which was purified directly by MDAP (high pH) to give N-((1r,4r)-4-(3-chloro-4-cyanophenoxy)cyclohexyl)-6-(4-(2-(4-(4-(2,4-dioxotetrahydropyrimidin-1(2H)-yl)-1H-indol-1-yl)piperidin-1-yl)-2-oxoethyl)piperazin-1-yl)pyridazine-3-carboxamide (42 mg, 0.053 mmol, 12.12% yield). LCMS (high pH A): Rt=1.10 min, MH⁺=793.2

### Example AR135

### N-((1r,4r)-4-(3-Chloro-4-cyanophenoxy)cyclohexyl)-6-(4-(1-(4-(4-(2,4-dioxotetrahydropyrimidin-1(2H)-yl)-1H-pyrrolo[2,3-c]pyridin-1-yl)piperidin-1-yl)ethyl)piperidin-1-yl)pyridazine-3-carboxamide (AR135)

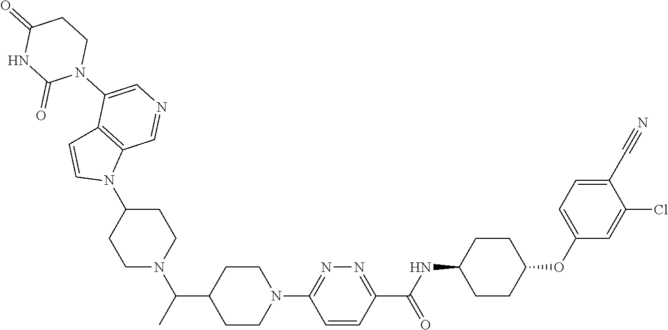 [SMILES: CC(C1CCN(c2ccc(C(=O)N[C@H]3CC[C@H](Oc4ccc(C#N)c(Cl)c4)CC3)nn2)CC1)N1CCC(n2ccc3c(N4CCC(=O)NC4=O)cncc32)CC1]

1-(1-(Piperidin-4-yl)-1H-pyrrolo[2,3-c]pyridin-4-yl)dihydropyrimidine-2,4(1H,3H)-dione (may be prepared as described in Example ECB6; 42.9 mg, 0.137 mmol), 6-(4-acetylpiperidin-1-yl)-N-((1r,4r)-4-(3-chloro-4-cyanophenoxy)cyclohexyl)pyridazine-3-carboxamide (may be prepared as described in Description 206; 60 mg, 0.124 mmol) and tetraisopropoxytitanium (0.048 mL, 0.162 mmol) were stirred in DCM (2 ml) at room temperature for 1 h. Sodium triacetoxyborohydride (52.8 mg, 0.249 mmol) was added, and the mixture was stirred for 16 h. A further portion of tetraisopropoxytitanium (0.048 mL, 0.162 mmol) was added, and the mixture was stirred for a further 4 h, then was heated to 50° C. for 21 h. A further portion of tetraisopropoxytitanium (0.111 mL, 0.373 mmol) was added, and the reaction was stirred for 6 h. The mixture was cooled to room temperature and was left to stand overnight. The reaction was diluted with DCM (3 mL) and water (5 mL), then saturated sodium bicarbonate (10 mL), and a further portion of DCM (10 mL) was added. The mixture was further diluted with HCl (10 mL), and the layers were separated. The aqueous layer was extracted with DCM (3×15 mL), and the pH of the combined organic layers was adjusted to pH 9 by the addition of saturated sodium bicarbonate. The layers were separated, and the aqueous layer was extracted with DCM (3×15 mL), and the combined organic layers were concentrated in vacuo. The crude product was purified by MDAP (high pH) to give N-((1r,4r)-4-(3-chloro-4-cyanophenoxy)cyclohexyl)-6-(4-(1-(4-(4-(2,4-dioxotetrahydropyrimidin-1(2H)-yl)-1H-pyrrolo[2,3-c]pyridin-1-yl)piperidin-1-yl)ethyl)piperidin-1-yl)pyridazine-3-carboxamide (5.2 mg, 6.67 μmol, 5.36% yield). LCMS (high pH A): Rt=1.23 min, MH⁺=779.2

### Examples AR136 and AR137

### N-((1r,4R)-4-(3-Chloro-4-cyanophenoxy)cyclohexyl)-6-((R)-3-((4-(4-(2,4-dioxotetrahydropyrimidin-1(2H)-yl)-1H-indazol-1-yl)piperidin-1-yl)methyl)piperidin-1-yl)pyridazine-3-carboxamide (AR136)

### N-((1r,4R)-4-(3-Chloro-4-cyanophenoxy)cyclohexyl)-6-((S)-3-((4-(4-(2,4-dioxotetrahydropyrimidin-1(2H)-yl)-1H-indazol-1-yl)piperidin-1-yl)methyl)piperidin-1-yl)pyridazine-3-carboxamide (AR137)

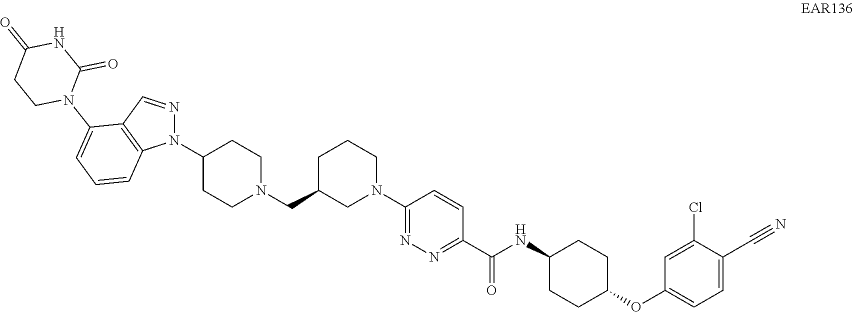 [SMILES: N#Cc1ccc(O[C@H]2CC[C@H](NC(=O)c3ccc(N4CCC[C@H](CN5CCC(n6ncc7c(N8CCC(=O)NC8=O)cccc76)CC5)C4)nn3)CC2)cc1Cl]

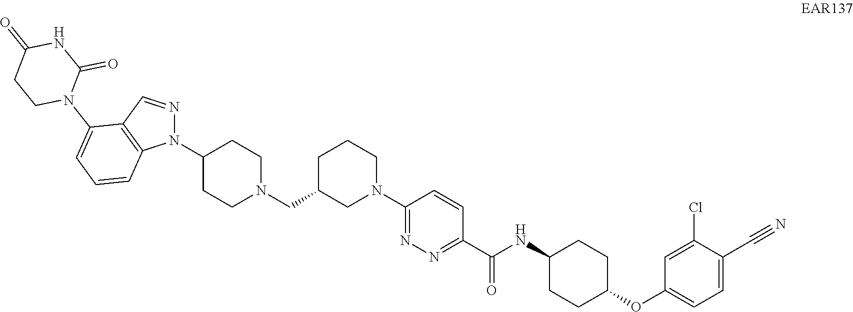 [SMILES: N#Cc1ccc(O[C@H]2CC[C@H](NC(=O)c3ccc(N4CCC[C@@H](CN5CCC(n6ncc7c(N8CCC(=O)NC8=O)cccc76)CC5)C4)nn3)CC2)cc1Cl]

1-(1-(Piperidin-4-yl)-1H-indazol-4-yl)dihydropyrimidine-2,4(1H,3H)-dione, trifluoroacetic acid salt (may be prepared as described in Example ECB4; 90 mg, 0.211 mmol) was suspended in DCM (20 ml) at room temperature. DIPEA (0.037 mL, 0.211 mmol) was added, followed by N-((1r,4r)-4-(3-chloro-4-cyanophenoxy)cyclohexyl)-6-(3-formylpiperidin-1-yl)pyridazine-3-carboxamide (may be prepared as described Description 102; 90 mg, 0.192 mmol) and the mixture was stirred for 1 h. Sodium triacetoxyborohydride (89 mg, 0.421 mmol) was added, and the mixture was stirred for 2 h. Sodium bicarbonate solution (20 ml) was added, and the mixture was stirred for 20 min. The phases were separated, and the organic layer dried and evaporated in vacuo to give a colourless solid. The crude compound was dissolved in DCM and loaded onto a 24 g silica column, then eluted with 0-20% 2M methanolic ammonia in DCM to give N-((1r,4r)-4-(3-chloro-4-cyanophenoxy)cyclohexyl)-6-(3-((4-(4-(2,4-dioxotetrahydropyrimidin-1(2H)-yl)-1H-indazol-1-yl)piperidin-1-yl)methyl)piperidin-1-yl)pyridazine-3-carboxamide (108 mg, 0.141 mmol, 67.0% yield). This material was chirally separated through a Chiralpak IE column (250×35 mm, 5 μm), eluting with 3:1 MeOH:MeCN (0.2% isopropylamine) to give first-eluting isomer N-((1r,4R)-4-(3-chloro-4-cyanophenoxy)cyclohexyl)-6-((R)-3-((4-(4-(2,4-dioxotetrahydropyrimidin-1(2H)-yl)-1H-indazol-1-yl)piperidin-1-yl)methyl)piperidin-1-yl)pyridazine-3-carboxamide (32.3 mg, 0.042 mmol) LCMS (high pH A): Rt=1.26 min, MH⁺=765.2; and second-eluting isomer N-((1r,4R)-4-(3-chloro-4-cyanophenoxy)cyclohexyl)-6-((S)-3-((4-(4-(2,4-dioxotetrahydropyrimidin-1(2H)-yl)-1H-indazol-1-yl)piperidin-1-yl)methyl)piperidin-1-yl)pyridazine-3-carboxamide (26.4 mg, 0.034 mmol) LCMS (high pH A): Rt=1.26 min, MH⁺=763.4, 765.3

### Example AR138

### (2S,5R)-4-(3-Chloro-4-cyanophenyl)-N-(6-(4-((4-(4-(2,4-dioxotetrahydropyrimidin-1(2H)-yl)-1H-indazol-1-yl)piperidin-1-yl)methyl)piperidin-1-yl)pyridin-3-yl)-2,5-dimethylpiperazine-1-carboxamide, formic acid salt (EAR138)

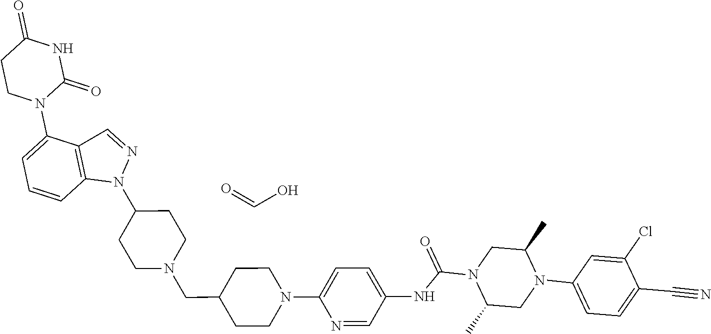 [SMILES: C[C@@H]1CN(C(=O)Nc2ccc(N3CCC(CN4CCC(n5ncc6c(N7CCC(=O)NC7=O)cccc65)CC4)CC3)nc2)[C@@H](C)CN1c1ccc(C#N)c(Cl)c1 ; O=CO]

A mixture of 1-(1-(piperidin-4-yl)-1H-indazol-4-yl)dihydropyrimidine-2,4(1H,3H)-dione, trifluoroacetic acid salt (may be prepared as described in Example ECB4; 41 mg, 0.096 mmol) and (2S,5R)-4-(3-chloro-4-cyanophenyl)-N-(6-(4-formylpiperidin-1-yl)pyridin-3-yl)-2,5-dimethylpiperazine-1-carboxamide (may be prepared as described in Description 209; 85 mg, 0.177 mmol) was stirred in DCM (6 mL) at room temperature for 0.5 h. Sodium triacetoxyborohydride (83 mg, 0.392 mmol) was added. After 2.5 h, the reaction was partitioned between DCM (25 mL) and saturated sodium hydrogen carbonate (25 mL). The organic phase was separated. The aqueous phase was extracted with DCM (10 mL×2). The combined organic phases were washed with water (30 mL) and brine (30 mL), dried and evaporated. The residue was purified by formic acid MDAP (formic) to give (2S,5R)-4-(3-chloro-4-cyanophenyl)-N-(6-(4-((4-(4-(2,4-dioxotetrahydropyrimidin-1(2H)-yl)-1H-indazol-1-yl)piperidin-1-yl)methyl)piperidin-1-yl)pyridin-3-yl)-2,5-dimethylpiperazine-1-carboxamide, formic acid salt (24.2 mg, 0.029 mmol, 30.6% yield). LCMS (high pH A): Rt=1.16 min, MH⁺=778.2

### Example AR139

### 2-Chloro-4-((2S,5R)-4-(2-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyrimidine-5-carbonyl)-2,5-dimethylpiperazin-1-yl)benzonitrile, formic acid salt (AR139)

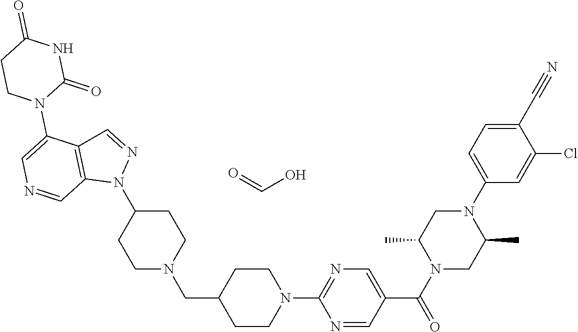 [SMILES: C[C@@H]1CN(c2ccc(C#N)c(Cl)c2)[C@@H](C)CN1C(=O)c1cnc(N2CCC(CN3CCC(n4ncc5c(N6CCC(=O)NC6=O)cncc54)CC3)CC2)nc1 ; O=CO]

A mixture of 1-(1-(piperidin-4-yl)-1H-pyrazolo[3,4-c]pyridin-4-yl)dihydropyrimidine-2,4(1H,3H)-dione (may be prepared as described in Example ECB8; 38 mg, 0.121 mmol) and 2-chloro-4-((2S,5R)-4-(2-(4-formylpiperidin-1-yl)pyrimidine-5-carbonyl)-2,5-dimethylpiperazin-1-yl)benzonitrile (may be prepared as described in Description 212; 56 mg, 0.120 mmol) was stirred in DCM (3 mL) at room temperature for 0.5 h. Sodium triacetoxyborohydride (76 mg, 0.360 mmol) was added. After 16 h, the reaction was partitioned between DCM (10 mL) and saturated sodium hydrogen carbonate (10 mL). The organic phase was separated. The aqueous phase was extracted with DCM (10 mL×2). The combined organic phases were washed with water (30 mL) and brine (30 mL), dried, evaporated and purified by MDAP (formic) to give 2-chloro-4-((2S,5R)-4-(2-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyrimidine-5-carbonyl)-2,5-dimethylpiperazin-1-yl)benzonitrile, formic acid salt (17.4 mg, 0.021 mmol, 17.88% yield). LCMS (high pH A): Rt=1.11 min, MH⁺=763.4, 765.3

### Example AR140

### N-((1r,4r)-4-(3-Chloro-4-cyanophenoxy)cyclohexyl)-6-(4-((4-(4-(2,4-dioxotetrahydropyrimidin-1(2H)-yl)-5-fluoro-1H-indazol-1-yl)piperidin-1-yl)methyl)piperidin-1-yl)pyridazine-3-carboxamide, hydrochloride (AR140)

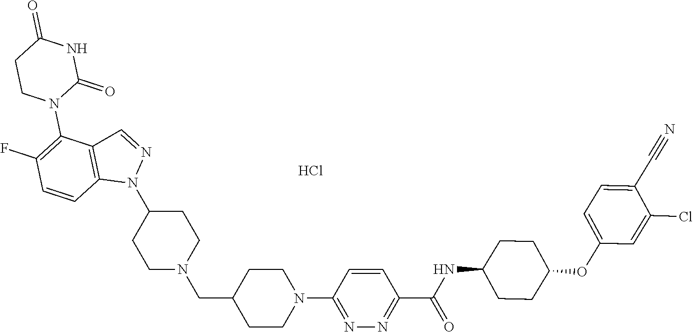 [SMILES: N#Cc1ccc(O[C@H]2CC[C@H](NC(=O)c3ccc(N4CCC(CN5CCC(n6ncc7c(N8CCC(=O)NC8=O)c(F)ccc76)CC5)CC4)nn3)CC2)cc1Cl ; Cl]

A mixture of 1-(5-fluoro-1-(piperidin-4-yl)-1H-indazol-4-yl)dihydropyrimidine-2,4(1H,3H)-dione (may be prepared as described in Example ECB21; 70 mg, 0.211 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 99 mg, 0.211 mmol) in DCM (1.5 mL) was stirred in a stoppered vessel at room temperature for 10 min. Sodium triacetoxyborohydride (67 mg, 0.316 mmol) was added, and the reaction was stirred 3 h. The mixture was washed sequentially with sat. NaHCO₃ (aq) (2.0 mL) and brine (2.0 mL), and the organic layer was passed through a hydrophobic frit. The filtrate was evaporated in vacuo, and the residue was purified through a XSelect CSH Prep C18 (150×30 mm, 5 μm), eluting with 45-100% MeCN in 10 mM NH₄HCO₃ in water (adjusted to pH 10 with aqueous NH₃) to give material that was subsequently was dissolved in 1,4-dioxane (0.5 mL) and DCM (2.0 mL), and treated with 4 M HCl in dioxane (0.050 mL, 0.201 mmol). The mixture was allowed to stir at room temperature for 2 h. The solvent was evaporated under a stream of nitrogen, and the solid was dried in a vacuum oven to give N-((1r,4r)-4-(3-chloro-4-cyanophenoxy)cyclohexyl)-6-(4-((4-(4-(2,4-dioxotetrahydropyrimidin-1(2H)-yl)-5-fluoro-1H-indazol-1-yl)piperidin-1-yl)methyl)piperidin-1-yl)pyridazine-3-carboxamide, hydrochloride (109 mg, 0.133 mmol, 62.9% yield). LCMS (high pH A): Rt=1.26 min, MH⁺=783

### Example AR141

### N-((1r,4r)-4-(3-Chloro-4-cyanophenoxy)cyclohexyl)-6-(4-((4-(4-(2,4-dioxo-3,4-dihydropyrimidin-1(2H)-yl)-1H-indol-1-yl)piperidin-1-yl)methyl)piperidin-1-yl)pyridazine-3-carboxamide, hydrochloride (EAR141)

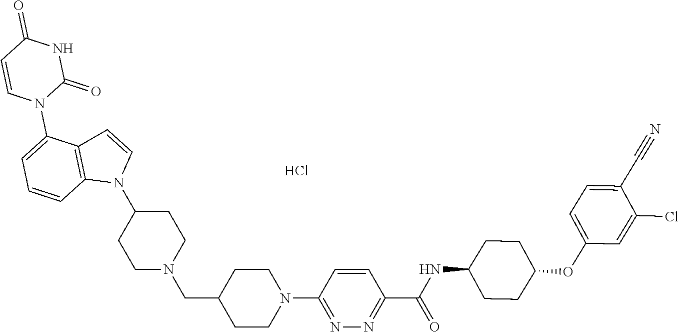 [SMILES: N#Cc1ccc(O[C@H]2CC[C@H](NC(=O)c3ccc(N4CCC(CN5CCC(n6ccc7c(-n8ccc(=O)[nH]c8=O)cccc76)CC5)CC4)nn3)CC2)cc1Cl ; Cl]

A mixture of 1-(1-(piperidin-4-yl)-1H-indol-4-yl)pyrimidine-2,4(1H,3H)-dione (may be prepared as described in Example ECB22; 53 mg, 0.171 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 80 mg, 0.171 mmol) in DCM (2.0 mL) was stirred in a stoppered vessel at room temperature for 10 min. Sodium triacetoxyborohydride (109 mg, 0.513 mmol) was added, and the reaction was stirred in a stoppered vessel at rt for 1 h. The mixture was washed sequentially with sat. NaHCO₃ (aq) (2.0 mL) and brine (2.0 mL), and the organic layer was passed through a hydrophobic frit. The filtrate was evaporated in vacuo and the residue was purified by Mass Directed AutoPrep on Xselect CSH C18 column eluting with a gradient of solvents 10 mM ammonium carbonate in water (adjusted to pH10 with ammonia solution) in acetonitrile to give a white solid as the free base (25 mg). The solid was dissolved in 1,4-Dioxane (0.5 mL) and DCM (2.0 mL) and treated with 4 M HCl in dioxane (0.012 mL, 0.049 mmol). The mixture was allowed to stir at room temperature in a stoppered vessel for 2 h. The solvent was evaporated under a stream of nitrogen and dried in a vacuum oven to give N-((1r,4r)-4-(3-chloro-4-cyanophenoxy)cyclohexyl)-6-(4-((4-(4-(2,4-dioxo-3,4-dihydropyrimidin-1(2H)-yl)-1H-indol-1-yl)piperidin-1-yl)methyl)piperidin-1-yl)pyridazine-3-carboxamide hydrochloride (22 mg, 0.028 mmol, 16.11% yield). LCMS (high pH A): Rt=1.26 min, MH⁺=762, 763, 764

### Examples AR142 and AR143

### N-((1r,4R)-4-(3-chloro-4-cyanophenoxy)cyclohexyl)-6-((R)-3-((4-(4-(2,4-dioxotetrahydropyrimidin-1(2H)-yl)-1H-indol-1-yl)piperidin-1-yl)methyl)piperidin-1-yl)pyridazine-3-carboxamide (EAR142)

### N-((1r,4R)-4-(3-chloro-4-cyanophenoxy)cyclohexyl)-6-((S)-3-((4-(4-(2,4-dioxotetrahydropyrimidin-1(2H)-yl)-1H-indol-1-yl)piperidin-1-yl)methyl)piperidin-1-yl)pyridazine-3-carboxamide (EAR143)

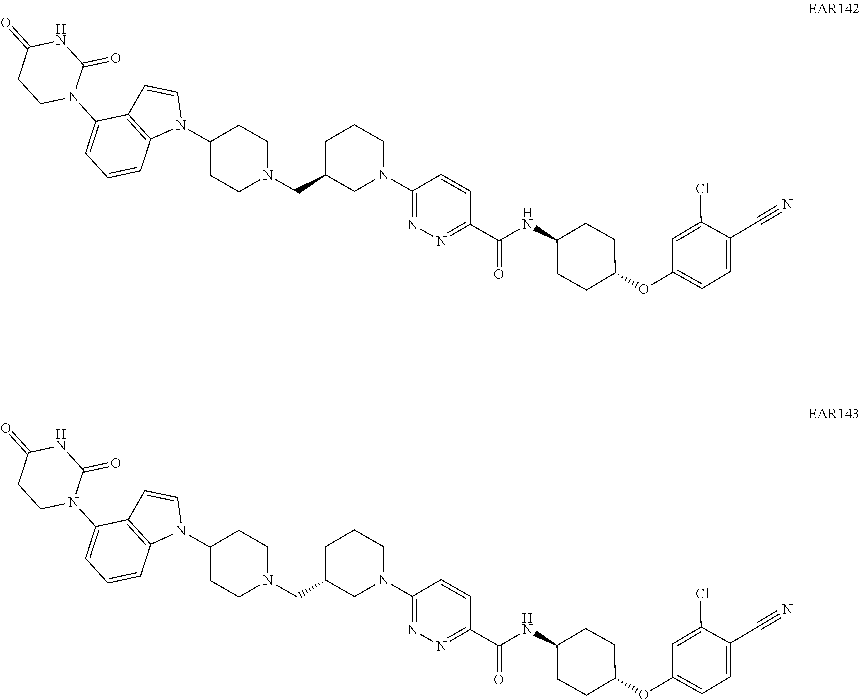 [SMILES: N#Cc1ccc(O[C@H]2CC[C@H](NC(=O)c3ccc(N4CCC[C@H](CN5CCC(n6ccc7c(N8CCC(=O)NC8=O)cccc76)CC5)C4)nn3)CC2)cc1Cl ; N#Cc1ccc(O[C@H]2CC[C@H](NC(=O)c3ccc(N4CCC[C@@H](CN5CCC(n6ccc7c(N8CCC(=O)NC8=O)cccc76)CC5)C4)nn3)CC2)cc1Cl]

1-(1-(Piperidin-4-yl)-1H-indol-4-yl)dihydropyrimidine-2,4(1H,3H)-dione (may be prepared as described in Example ECB1; 80 mg, 0.256 mmol) and N-((1r,4r)-4-(3-chloro-4-cyanophenoxy)cyclohexyl)-6-(3-formylpiperidin-1-yl)pyridazine-3-carboxamide (may be prepared as described in Description 102; 90 mg, 0.192 mmol) were suspended in DCM (20 ml) at room temperature. Sodium triacetoxyborohydride (109 mg, 0.512 mmol) was added, and the mixture was stirred for 1 h. More sodium triacetoxyborohydride (109 mg, 0.512 mmol) was added, and the mixture stirred for 2 h. Sodium bicarbonate solution (20 ml) was added, and the mixture stirred for 20 min. The phases were separated, and the organic layer was dried, evaporated in vacuo, dissolved in DCM and loaded onto a 24 g silica column, eluting with 0-20% 2M methanolic ammonia in DCM to give N-((1r,4r)-4-(3-chloro-4-cyanophenoxy)cyclohexyl)-6-(3-((4-(4-(2,4-dioxotetrahydropyrimidin-1(2H)-yl)-1H-indol-1-yl)piperidin-1-yl)methyl)piperidin-1-yl)pyridazine-3-carboxamide (110 mg, 0.144 mmol, 56.2% yield). This material was chirally separated through a Regis Whelk-O 1R,R column (250×30 mm, 5 μm), eluting with MeCN (0.2% isopropylamine) to give first-eluting isomer N-((1r,4R)-4-(3-chloro-4-cyanophenoxy)cyclohexyl)-6-((R)-3-((4-(4-(2,4-dioxotetrahydropyrimidin-1(2H)-yl)-1H-indol-1-yl)piperidin-1-yl)methyl)piperidin-1-yl)pyridazine-3-carboxamide (12.2 mg, 0.016 mmol), LCMS (high pH A): Rt=1.33 min, MH⁺=762.5, 764.3; and second-eluting isomer N-((1r,4S)-4-(3-chloro-4-cyanophenoxy)cyclohexyl)-6-((S)-3-((4-(4-(2,4-dioxotetrahydropyrimidin-1(2H)-yl)-1H-indol-1-yl)piperidin-1-yl)methyl)piperidin-1-yl)pyridazine-3-carboxamide (19.7 mg, 0.026 mmol), LCMS (high pH A): Rt=1.33 min, MH⁺=764.3

### Example AR144

### N-((1r,4r)-4-(3-Chloro-4-cyanophenoxy)cyclohexyl)-6-(4-(((3R,4R)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide, hydrochloride (EAR144)

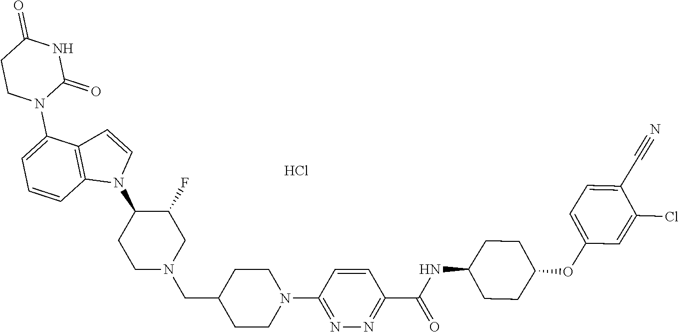 [SMILES: N#Cc1ccc(O[C@H]2CC[C@H](NC(=O)c3ccc(N4CCC(CN5CC[C@@H](n6ccc7c(N8CCC(=O)NC8=O)cccc76)[C@H](F)C5)CC4)nn3)CC2)cc1Cl ; Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 74 mg, 0.158 mmol) and 1-(1-((3R,4R)-3-fluoropiperidin-4-yl)-1H-indol-4-yl)dihydropyrimidine-2,4(1H,3H)-dione (may be prepared as described in Example ECB23; 43 mg, 0.130 mmol) were suspended in DCM (1.9 mL), and the mixture was stirred at room temperature under nitrogen for 10 min. Sodium triacetoxyborohydride (83 mg, 0.390 mmol) was added, and the reaction was stirred at RT for 15 hr. The mixture was diluted with DCM (20 mL), NaHCO₃ (15 mL) and brine (5 mL), and shaken vigorously. The layers were separated, and the aqueous was extracted with DCM (10 mL). The organics were combined, dried by passing through a hydrophobic frit, concentrated, dissolved in DMSO:methanol 1:1 (2 mL) and purified by MDAP (high pH) to give N-((1r,4r)-4-(3-chloro-4-cyanophenoxy)cyclohexyl)-6-(4-(((3R,4R)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide (64.2 mg, 0.082 mmol). To this material in dioxane (400 μL) was added HCl (4 M in dioxane) (0.051 mL, 0.205 mmol), and the resulting suspension was stirred at room temperature for 2 h. The solvent was removed under a stream of nitrogen then further dried in a vacuum oven to give N-((1r,4r)-4-(3-chloro-4-cyanophenoxy)cyclohexyl)-6-(4-(((3R,4R)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide hydrochloride (67.4 mg, 0.082 mmol, 63.2% yield). LCMS (high pH A): Rt=1.29 min, MH⁺=782.3, 784.3

### Example AR145

### N-((1r,4r)-4-(3-Chloro-4-cyanophenoxy)cyclohexyl)-6-(4-(((3S,4S)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide, hydrochloride (AR145)

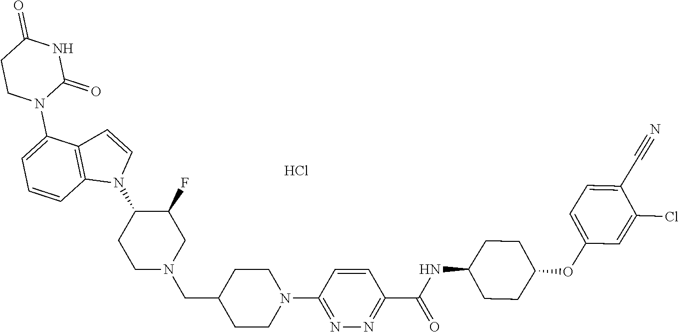 [SMILES: N#Cc1ccc(O[C@H]2CC[C@H](NC(=O)c3ccc(N4CCC(CN5CC[C@H](n6ccc7c(N8CCC(=O)NC8=O)cccc76)[C@@H](F)C5)CC4)nn3)CC2)cc1Cl ; Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 100 mg, 0.214 mmol) and 1-(1-((3S,4S)-3-fluoropiperidin-4-yl)-1H-indol-4-yl)dihydropyrimidine-2,4(1H,3H)-dione (may be prepared as described in Example ECB24; 59 mg, 0.179 mmol) were suspended in DCM (2.5 mL), and the mixture was stirred at room temperature under nitrogen for 10 min. Sodium triacetoxyborohydride (114 mg, 0.536 mmol) was added, and the reaction was stirred at room temperature for 16 h. The mixture was diluted with DCM (20 mL), NaHCO₃ (15 mL) and brine (5 mL), and shaken vigorously. The layers were separated, and the aqueous was extracted with DCM (10 mL). The organics were combined, dried by passing through a hydrophobic frit, concentrated, dissolved in DMSO:methanol 3:1 (2 mL) and purified by MDAP (high h pH) to give N-((1r,4r)-4-(3-chloro-4-cyanophenoxy)cyclohexyl)-6-(4-(((3S,4S)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide (72.4 mg, 0.093 mmol). To this material in dioxane (500 μL) was added HCl (4 M in dioxane) (0.058 mL, 0.233 mmol), and the resulting suspension was stirred at room temperature for 1 h. The solvent was removed under a stream of nitrogen then further dried in a vacuum oven to give N-((1r,4r)-4-(3-chloro-4-cyanophenoxy)cyclohexyl)-6-(4-(((3S,4S)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide hydrochloride (71.4 mg, 0.087 mmol, 48.8% yield). LCMS (high pH A): Rt=1.29 min, MH⁺=782.3, 784.2

### Example AR146

### 2-Chloro-4-((2S,5R)-4-(6-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyridazine-3-carbonyl)-2,5-dimethylpiperazin-1-yl)benzonitrile (AR146)

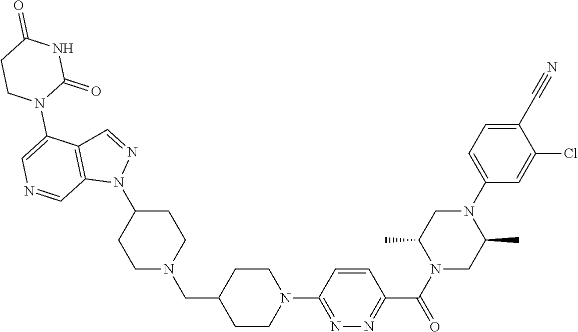 [SMILES: C[C@@H]1CN(c2ccc(C#N)c(Cl)c2)[C@@H](C)CN1C(=O)c1ccc(N2CCC(CN3CCC(n4ncc5c(N6CCC(=O)NC6=O)cncc54)CC3)CC2)nn1]

A mixture of 1-(1-(piperidin-4-yl)-1H-pyrazolo[3,4-c]pyridin-4-yl)dihydropyrimidine-2,4(1H,3H)-dione (may be prepared as described in Example ECB8; 63 mg, 0.200 mmol) and 2-chloro-4-((2S,5R)-4-(6-(4-formylpiperidin-1-yl)pyridazine-3-carbonyl)-2,5-dimethylpiperazin-1-yl)benzonitrile (may be prepared as described in Description 227; 78 mg, 0.168 mmol) was stirred in DCM (6 mL) at room temperature for 0.5 h. Sodium triacetoxyborohydride (114 mg, 0.538 mmol) was added, and the reaction was stirred at for 2.5 h. The mixture was partitioned between DCM (25 mL) and saturated sodium hydrogen carbonate (25 mL). The organic phase was separated. The aqueous phase was extracted with DCM (10 mL×2). The combined organic phases were washed with water (30 mL) and brine (30 mL). The organic phase was dried and evaporated, and the residue was purified by MDAP (high pH) to give 2-chloro-4-((2S,5R)-4-(6-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyridazine-3-carbonyl)-2,5-dimethylpiperazin-1-yl)benzonitrile (43.6 mg, 0.057 mmol, 33.9% yield). LCMS (high pH A): Rt=1.05 min, MH⁺=763.3, 765.2

### Example AR147

### 2-Chloro-4-((2S,5R)-4-(5-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)pyrazine-2-carbonyl)-2,5-dimethylpiperazin-1-yl)benzonitrile (AR147)

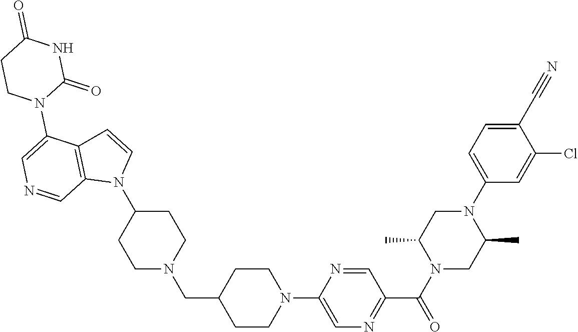 [SMILES: C[C@@H]1CN(c2ccc(C#N)c(Cl)c2)[C@@H](C)CN1C(=O)c1cnc(N2CCC(CN3CCC(n4ccc5c(N6CCC(=O)NC6=O)cncc54)CC3)CC2)cn1]

A mixture of 1-(1-(piperidin-4-yl)-1H-pyrrolo[2,3-c]pyridin-4-yl)dihydropyrimidine-2,4(1H,3H)-dione (may be prepared as described in Example ECB6; 104 mg, 0.332 mmol) and 2-chloro-4-((2S,5R)-4-(5-(4-formylpiperidin-1-yl)pyrazine-2-carbonyl)-2,5-dimethylpiperazin-1-yl)benzonitrile (may be prepared as described in Description 230; 128 mg, 0.274 mmol) was stirred in DCM (6 mL) for 0.5 h. Sodium triacetoxyborohydride (186 mg, 0.878 mmol) was added, and after 3.5 h mixture was partitioned between DCM (25 mL) and saturated sodium hydrogen carbonate (25 mL). The organic phase was separated, and the aqueous phase was extracted with DCM (15 mL×2). The combined organic phases were washed with water (25 mL) and brine (25 mL×2), concentrated and purified by MDAP (high pH) to give 2-chloro-4-((2S,5R)-4-(5-(4-((4-(4-(2,4-dioxotetrahydropyrimidin-1(2H)-yl)-1H-pyrrolo[2,3-c]pyridin-1-yl)piperidin-1-yl)methyl)piperidin-1-yl)pyrazine-2-carbonyl)-2,5-dimethylpiperazin-1-yl)benzonitrile (26 mg, 0.034 mmol, 12.41% yield). LCMS (high pH A): Rt=1.11 min, MH⁺=764.3

### Example AR148

### N-((1r,4r)-4-(3-Chloro-4-cyanophenoxy)cyclohexyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyridazine-3-carboxamide (AR148)

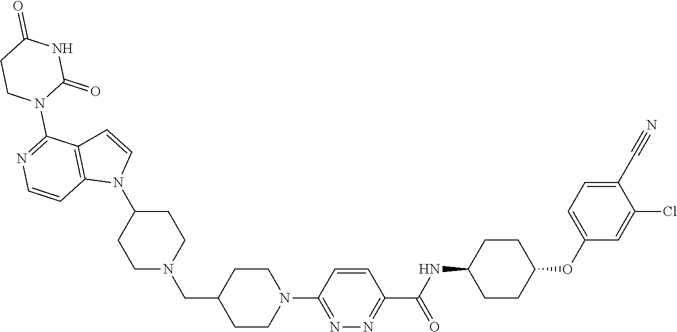 [SMILES: N#Cc1ccc(O[C@H]2CC[C@H](NC(=O)c3ccc(N4CCC(CN5CCC(n6ccc7c(N8CCC(=O)NC8=O)nccc76)CC5)CC4)nn3)CC2)cc1Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 71.7 mg, 0.153 mmol) and 1-(1-(piperidin-4-yl)-1H-pyrrolo[3,2-c]pyridin-4-yl)dihydropyrimidine-2,4(1H,3H)-dione (may be prepared as described in Example ECB25; 40.0 mg, 0.128 mmol) were stirred in DCM (2 mL) for 10 min. Sodium triacetoxyborohydride (81 mg, 0.383 mmol) was added, and the reaction was stirred overnight (ca 24 h). The mixture was diluted with DCM, and NaHCO₃ and brine were added, with shaking. The reaction was passed through a hydrophobic frit and blown down. The material was purified by MDAP (high pH) to give N-((1r,4r)-4-(3-chloro-4-cyanophenoxy)cyclohexyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyridazine-3-carboxamide (46 mg, 0.057 mmol, 44.7% yield). LCMS (high pH A): Rt=1.19 min, MH⁺=765.3

### Example AR149

### N-((1r,4r)-4-(3-Chloro-4-cyanophenoxy)cyclohexyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyridazine-3-carboxamide (AR149)

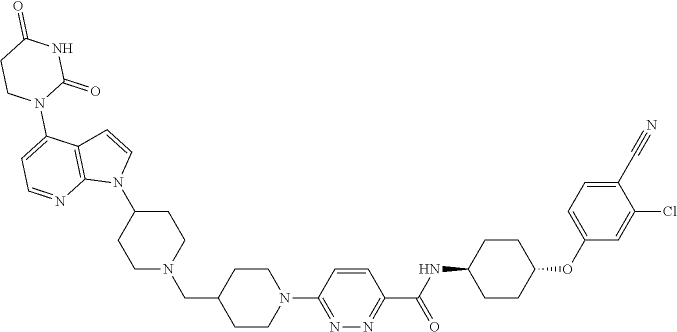 [SMILES: N#Cc1ccc(O[C@H]2CC[C@H](NC(=O)c3ccc(N4CCC(CN5CCC(n6ccc7c(N8CCC(=O)NC8=O)ccnc76)CC5)CC4)nn3)CC2)cc1Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 71.7 mg, 0.153 mmol) and 1-(1-(piperidin-4-yl)-1H-pyrrolo[2,3-b]pyridin-4-yl)dihydropyrimidine-2,4(1H,3H)-dione (may be prepared as described in Example ECB26; 40.0 mg, 0.128 mmol) were stirred in DCM (2 mL) for 10 min. Sodium triacetoxyborohydride (81 mg, 0.383 mmol) was added, and the reaction was stirred overnight (ca 24 h). The mixture was diluted with DCM, and NaHCO₃ and brine were added, with shaking. The reaction was passed through a hydrophobic frit and blown down. The material was purified by MDAP (high pH) to give N-((1r,4r)-4-(3-chloro-4-cyanophenoxy)cyclohexyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyridazine-3-carboxamide (35 mg, 0.043 mmol, 34.0% yield). LCMS (high pH A): Rt=1.21 min, MH⁺=765.3

### Example AR150

### 2-Chloro-4-((2S,5R)-4-(5-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyrazine-2-carbonyl)-2,5-dimethylpiperazin-1-yl)benzonitrile (AR150)

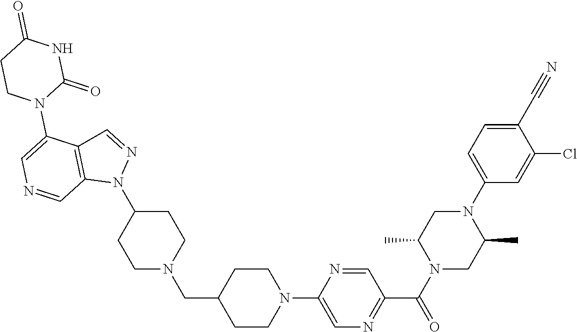 [SMILES: C[C@@H]1CN(c2ccc(C#N)c(Cl)c2)[C@@H](C)CN1C(=O)c1cnc(N2CCC(CN3CCC(n4ncc5c(N6CCC(=O)NC6=O)cncc54)CC3)CC2)cn1]

A mixture of 1-(1-(piperidin-4-yl)-1H-pyrazolo[3,4-c]pyridin-4-yl)dihydropyrimidine-2,4(1H,3H)-dione (may be prepared as described in Example ECB8; 105 mg, 0.334 mmol) and 2-chloro-4-((2S,5R)-4-(5-(4-formylpiperidin-1-yl)pyrazine-2-carbonyl)-2,5-dimethylpiperazin-1-yl)benzonitrile be (may prepared as described in Description 230; 128 mg, 0.274 mmol) was stirred in DCM (6 mL) for 0.5 h. Sodium triacetoxyborohydride (194 mg, 0.915 mmol) was added, and after 3.5 h the reaction was partitioned between DCM (25 mL) and saturated sodium hydrogen carbonate (25 mL). The organic phase was separated, and the aqueous phase was extracted with DCM (15 mL×2). The combined organic phases were washed with water (25 mL) and brine (25 mL×2), dried, evaporated and purified by MDAP (high pH) to give 2-chloro-4-((2S,5R)-4-(5-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyrazine-2-carbonyl)-2,5-dimethylpiperazin-1-yl)benzonitrile (46 mg, 0.060 mmol, 21.93% yield). LCMS (high pH A): Rt=1.11 min, MH⁺=765.2

### Examples AR151-AR154

In four separate reactions, to the amines (0.155 mmol) listed in the table below were added 6-chloro-N-((1r,4r)-4-(3-chloro-4-cyanophenoxy)cyclohexyl)pyridazine-3-carboxamide (may be prepared as described in Description 90; 0.061 g, 0.155 mmol) and DIPEA (0.135 mL, 0.775 mmol) dissolved in DMSO (2 mL). The reactions were left to stir 60° C. for 18 hr, injected as-is and purified by MDAP (formic) to give the following compounds:

| | Product structure and name | | Mass, |
|---|---|---|---|
| Example | Analytical | Amine | yield |
| EAR151 | 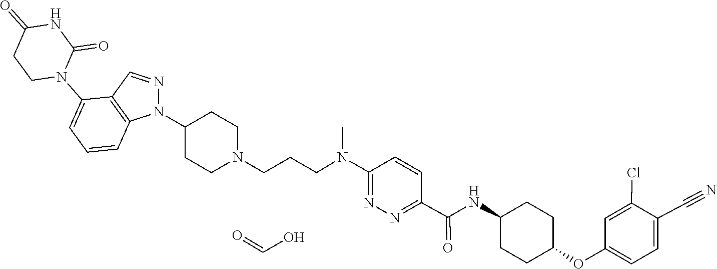 [SMILES: CN(CCCN1CCC(n2ncc3c(N4CCC(=O)NC4=O)cccc32)CC1)c1ccc(C(=O)N[C@H]2CC[C@H](Oc3ccc(C#N)c(Cl)c3)CC2)nn1 ; O=CO] | ECBL8 | 44 mg, 32.5% |
| | N-((1r,4r)-4-(3-Chloro-4-cyanophenoxy)cyclohexyl)-6-((3-(4-(4- | | |
| | (2,4-dioxotetrahydropyrimidin-1(2H)-yl)-1H-indazol-1- | | |
| | yl)piperidin-1-yl)propyl)(methyl)amino)pyridazine-3- | | |
| | carboxamide, formic acid salt | | |
| | LCMS (formic A): Rt = 0.78 min, MH⁺ = 739.59 | | |
| EAR152 | 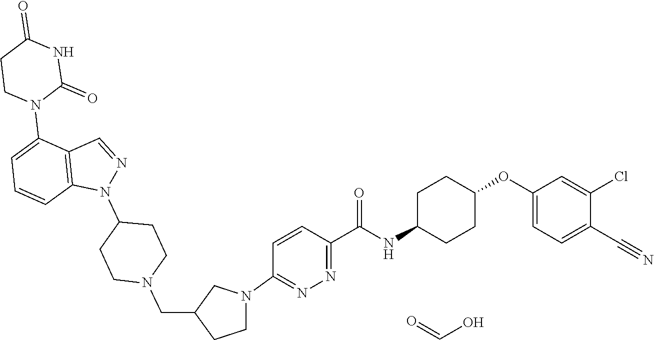 [SMILES: N#Cc1ccc(O[C@H]2CC[C@H](NC(=O)c3ccc(N4CCC(CN5CCC(n6ncc7c(N8CCC(=O)NC8=O)cccc76)CC5)C4)nn3)CC2)cc1Cl ; O=CO] | ECBL9 | 89.8 mg, 65.4% |
| | N-((1r,4r)-4-(3-Chloro-4-cyanophenoxy)cyclohexyl)-6-(3-((4-(4- | | |
| | (2,4-dioxotetrahydropyrimidin-1(2H)-yl)-1H-indazol-1- | | |
| | yl)piperidin-1-yl)methyl)pyrrolidin-1-yl)pyridazine-3- | | |
| | carboxamide, formic acid salt | | |
| | LCMS (formic A): Rt = 0.77 min, MH⁺ = 751.63 | | |
| EAR153 | 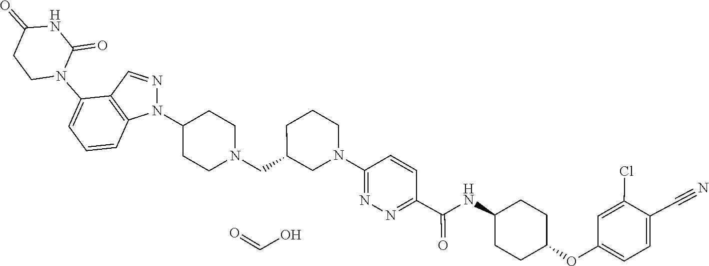 [SMILES: N#Cc1ccc(O[C@H]2CC[C@H](NC(=O)c3ccc(N4CCC[C@@H](CN5CCC(n6ncc7c(N8CCC(=O)NC8=O)cccc76)CC5)C4)nn3)CC2)cc1Cl ; O=CO] | ECBL10 | 78.1 mg, 55.9% |
| | N-((1r,4S)-4-(3-Chloro-4-cyanophenoxy)cyclohexyl)-6-((S)-3-((4- | | |
| | (4-(2,4-dioxotetrahydropyrimidin-1(2H)-yl)-1H-indazol-1- | | |
| | yl)piperidin-1-yl)methyl)piperidin-1-yl)pyridazine-3-carboxamide, | | |
| | formic acid salt | | |
| | LCMS (formic A): Rt = 0.81 min, MH⁺ = 765.61 | | |
| EAR154 | 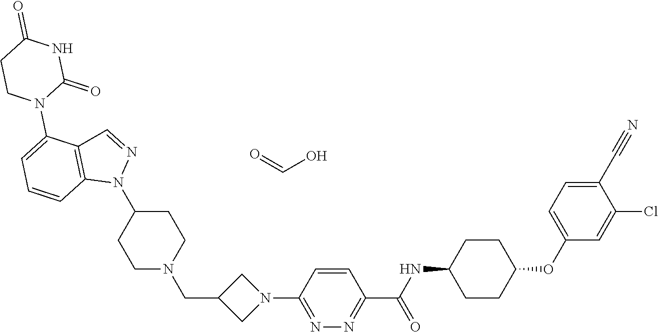 [SMILES: N#Cc1ccc(O[C@H]2CC[C@H](NC(=O)c3ccc(N4CC(CN5CCC(n6ncc7c(N8CCC(=O)NC8=O)cccc76)CC5)C4)nn3)CC2)cc1Cl ; O=CO] | ECBL11 | 29.6 mg, 19.7% |
| | N-((1r,4r)-4-(3-Chloro-4-cyanophenoxy)cyclohexyl)-6-(3-((4-(4- | | |
| | (2,4-dioxotetrahydropyrimidin-1(2H)-yl)-1H-indazol-1- | | |
| | yl)piperidin-1-yl)methyl)azetidin-1-yl)pyridazine-3-carboxamide, | | |
| | formic acid salt | | |
| | LCMS (formic A): Rt = 0.76 min, MH⁺ = 737.6 | | |

### Example AR155

### N-((1r,4r)-4-(3-Chloro-4-cyanophenoxy)cyclohexyl)-6-(4-(((3S,4R)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide, hydrochloride (EAR155)

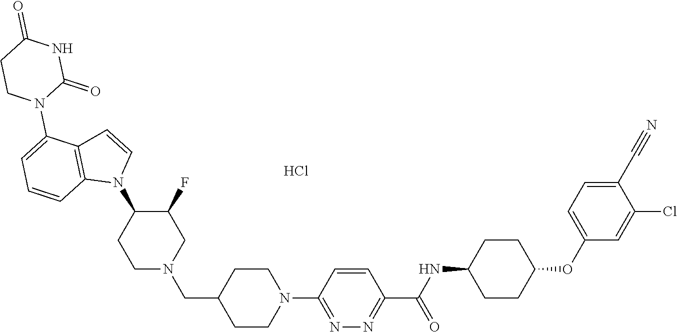 [SMILES: N#Cc1ccc(O[C@H]2CC[C@H](NC(=O)c3ccc(N4CCC(CN5CC[C@@H](n6ccc7c(N8CCC(=O)NC8=O)cccc76)[C@@H](F)C5)CC4)nn3)CC2)cc1Cl ; Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 93 mg, 0.200 mmol) and 1-(1-((3S,4R)-3-fluoropiperidin-4-yl)-1H-indol-4-yl)dihydropyrimidine-2,4(1H,3H)-dione (may be prepared as described in Example ECB27; 55 mg, 0.156 mmol) were suspended in DCM (2.4 mL), and the mixture was stirred at room temperature under nitrogen for 10 min. Sodium triacetoxyborohydride (106 mg, 0.499 mmol) was added, and the reaction was stirred at room temperature for 19 hr. The mixture was diluted with DCM (20 mL), NaHCO₃ (15 mL) and brine (5 mL), and shaken vigorously. The layers were separated, and the aqueous was extracted with DCM (10 mL). The organics were combined, dried by passing through a hydrophobic frit, concentrated, dissolved in DMSO:methanol 1:1 (2 mL) and purified by MDAP (high pH) to give N-((1r,4r)-4-(3-chloro-4-cyanophenoxy)cyclohexyl)-6-(4-(((3S,4R)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide (72.4 mg, 0.093 mmol). To this material in dioxane (300 μL) was added HCl (4 M in dioxane) (0.022 mL, 0.0875 mmol), and the resulting suspension was stirred at room temperature for 1.5 h. The solvent was removed under a stream of nitrogen then further dried in a vacuum oven to give N-((1r,4r)-4-(3-chloro-4-cyanophenoxy)cyclohexyl)-6-(4-(((3S,4R)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide hydrochloride (15.8 mg, 0.019 mmol, 11.59% yield). LCMS (high pH A): Rt=1.26 min, MH⁺=782.2, 784.2

### Example AR156

### N-((1r,4r)-4-(3-Chloro-4-cyanophenoxy)cyclohexyl)-6-(4-(((3R,4S)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide, hydrochloride (EAR156)

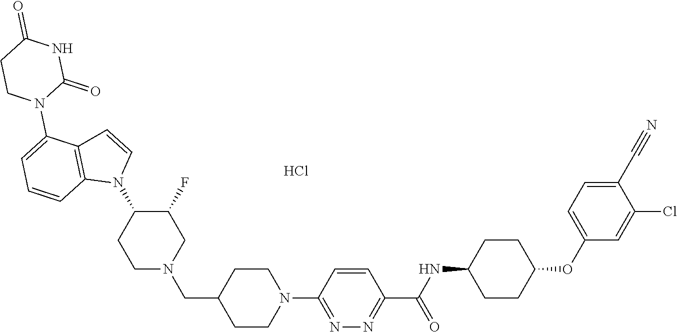 [SMILES: N#Cc1ccc(O[C@H]2CC[C@H](NC(=O)c3ccc(N4CCC(CN5CC[C@H](n6ccc7c(N8CCC(=O)NC8=O)cccc76)[C@H](F)C5)CC4)nn3)CC2)cc1Cl ; Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 93 mg, 0.200 mmol) and 1-(1-((3R,4S)-3-fluoropiperidin-4-yl)-1H-indol-4-yl)dihydropyrimidine-2,4(1H,3H)-dione (may be prepared as described in Example ECB28; 55 mg, 0.166 mmol) were suspended in DCM (2.4 mL), and the mixture was stirred at room temperature under nitrogen for 10 min. Sodium triacetoxyborohydride (106 mg, 0.499 mmol) was added, and the reaction was stirred at room temperature for 19 hr, during which time the solvent evaporated. DCM (2.4 mL) was added, and the reaction was stirred for 2 hr. The mixture was diluted with DCM (20 mL), NaHCO₃ (15 mL) and brine (5 mL), and shaken vigorously. The layers were separated, and the aqueous was extracted with DCM (10 mL). The organics were combined, dried by passing through a hydrophobic frit, concentrated, dissolved in DMSO:methanol 1:1 (1.5 mL) and left to stand at room temperature overnight. The solution had some cloudy solids in the bottom, so methanol (1 mL) was added, the mixture sonicated, forming a suspension, and the solids were collected by filtration, washing with methanol to give the crude product. This material was dissolved in DMSO (10 mL) and purified by MDAP (high pH) to give N-((1r,4r)-4-(3-chloro-4-cyanophenoxy)cyclohexyl)-6-(4-(((3R,4S)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide (72.4 mg, 0.093 mmol). To this material in dioxane (1.2 mL) was added HCl (4 M in dioxane) (0.058 mL, 0.233 mmol), and the resulting suspension was stirred at room temperature for 2 h. The solvent was removed under a stream of nitrogen then further dried in a vacuum oven to give N-((1r,4r)-4-(3-chloro-4-cyanophenoxy)cyclohexyl)-6-(4-(((3R,4S)-4-(4-(2,4-dioxotetrahydropyrimidin-1(2H)-yl)-1H-indol-1-yl)-3-fluoropiperidin-1-yl)methyl)piperidin-1-yl)pyridazine-3-carboxamide hydrochloride (76.5 mg, 0.093 mmol, 56.1% yield). LCMS (high pH A): Rt=1.26 min, MH⁺=782.2, 784.2

### Example AR157

### N-((1r,4r)-4-((3-Chloro-4-cyanophenyl)(methyl)amino)cyclohexyl)-6-(4-((4-(4-(2,4-dioxotetrahydropyrimidin-1(2H)-yl)-1H-indol-1-yl)piperidin-1-yl)methyl)piperidin-1-yl)pyridazine-3-carboxamide (EAR157)

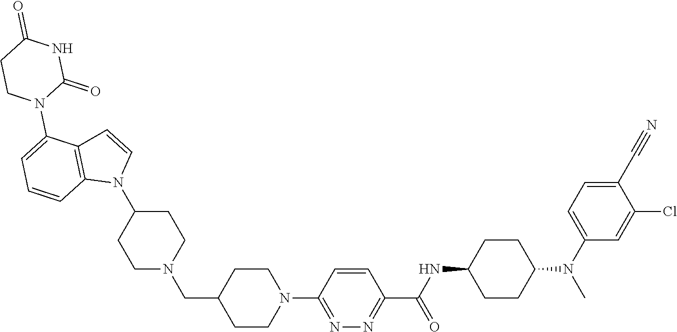 [SMILES: CN(c1ccc(C#N)c(Cl)c1)[C@H]1CC[C@H](NC(=O)c2ccc(N3CCC(CN4CCC(n5ccc6c(N7CCC(=O)NC7=O)cccc65)CC4)CC3)nn2)CC1]

To 1-(1-(1-(piperidin-4-ylmethyl)piperidin-4-yl)-1H-indol-4-yl)dihydropyrimidine-2,4(1H,3H)-dione hydrochloride (may be prepared as described in Example ECBL12; 55 mg, 0.114 mmol) was added 6-chloro-N-((1r,4r)-4-((3-chloro-4-cyanophenyl)(methyl)amino)cyclohexyl)pyridazine-3-carboxamide (may be prepared as described in Description 243; 50 mg, 0.124 mmol) and DMSO (0.5 mL) added. The vessel was sealed and stirred at 70° C. for 22 h. The reaction was allowed to cool to room temperature and evaporated under a stream of nitrogen. The residue was purified by MDAP (high pH) to give N-((1r,4r)-4-((3-chloro-4-cyanophenyl)(methyl)amino)cyclohexyl)-6-(4-((4-(4-(2,4-dioxotetrahydropyrimidin-1(2H)-yl)-1H-indol-1-yl)piperidin-1-yl)methyl)piperidin-1-yl)pyridazine-3-carboxamide (21 mg, 0.027 mmol). LCMS (high pH A): Rt=1.31 min, MH⁺=777

### Example AR158

### N-((1r,4r)-4-(3-Chloro-4-cyanophenoxy)cyclohexyl)-6-(4-(2-(4-(4-(2,4-dioxotetrahydropyrimidin-1(2H)-yl)-1H-indazol-1-yl)piperidin-1-yl)-2-oxoethyl)piperazin-1-yl)pyridazine-3-carboxamide (EAR158)

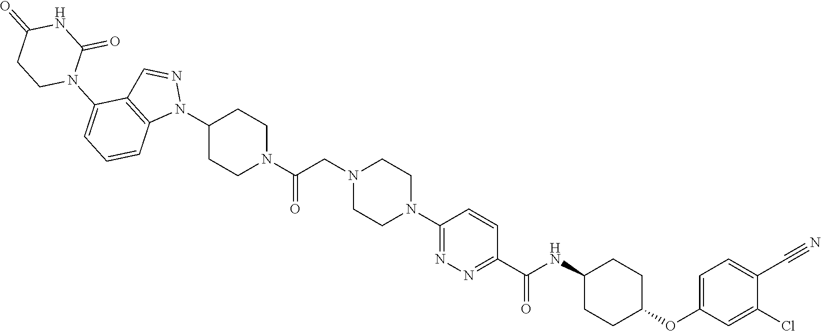 [SMILES: N#Cc1ccc(O[C@H]2CC[C@H](NC(=O)c3ccc(N4CCN(CC(=O)N5CCC(n6ncc7c(N8CCC(=O)NC8=O)cccc76)CC5)CC4)nn3)CC2)cc1Cl]

To a stirring solution of 2-(4-(6-(((1r,4r)-4-(3-chloro-4-cyanophenoxy)cyclohexyl)carbamoyl)pyridazin-3-yl)piperazin-1-yl) acetic acid (may be prepared as described in Description 204; 218 mg, 0.437 mmol) and 1-(1-(piperidin-4-yl)-1H-indol-4-yl)dihydropyrimidine-2,4(1H,3H)-dione (may be prepared as described in Example ECB1; 96 mg, 0.306 mmol; used substoichiometrically as the starting acid was approx 50% pure) and HATU (199 mg, 0.524 mmol) in N,N-Dimethylformamide (DMF) (3 mL) was added DIPEA (0.229 mL, 1.311 mmol), followed by stirring for 2 h. The solvent was partially removed using a flow of nitrogen gas until 1 mL of yellow coloured solution remained, which was purified directly by MDAP (high pH) to give N-((1r,4r)-4-(3-chloro-4-cyanophenoxy)cyclohexyl)-6-(4-(2-(4-(4-(2,4-dioxotetrahydropyrimidin-1(2H)-yl)-1H-indazol-1-yl)piperidin-1-yl)-2-oxoethyl)piperazin-1-yl)pyridazine-3-carboxamide (79 mg, 0.099 mmol, 22.76% yield). LCMS (high pH A): Rt=1.07 min, MH⁺=794.2

### Example AR159

### N-((1r,4r)-4-(3-Chloro-4-cyanophenoxy)cyclohexyl)-6-(4-(((S)-3-((4-(2,4-dioxotetrahydropyrimidin-1(2H)-yl)-1H-indol-1-yl)methyl)-3-fluoropiperidin-1-yl)methyl)piperidin-1-yl)pyridazine-3-carboxamide (EAR159)

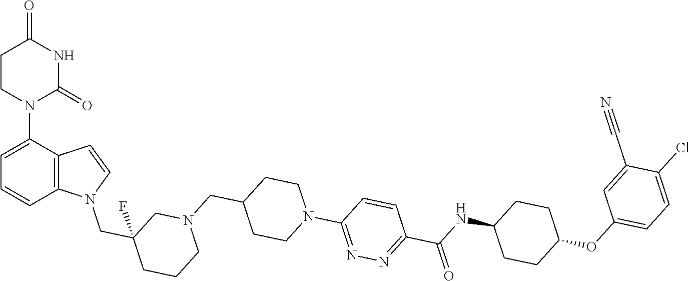 [SMILES: N#Cc1cc(O[C@H]2CC[C@H](NC(=O)c3ccc(N4CCC(CN5CCC[C@@](F)(Cn6ccc7c(N8CCC(=O)NC8=O)cccc76)C5)CC4)nn3)CC2)ccc1Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 50 mg, 0.107 mmol) and(S)-1-(1-((3-fluoropiperidin-3-yl)methyl)-1H-indol-4-yl)dihydropyrimidine-2,4(1H,3H)-dione (may be prepared as described in Example ECB29; 36.8 mg, 0.107 mmol) were dissolved in DCM (5 ml) and stirred for 30 minutes. Sodium triacetoxyborohydride (67.9 mg, 0.321 mmol) was added, and the mixture was stirred for 1 h, quenched with saturated sodium bicarbonate solution and stirred for 20 minutes. The organic layer was loaded onto a 24 g silica column and eluted with 0-20% 2M methanolic ammonia in DCM to give N-((1r,4r)-4-(3-chloro-4-cyanophenoxy)cyclohexyl)-6-(4-(((S)-3-((4-(2,4-dioxotetrahydropyrimidin-1(2H)-yl)-1H-indol-1-yl)methyl)-3-fluoropiperidin-1-yl)methyl)piperidin-1-yl)pyridazine-3-carboxamide (78 mg, 0.098 mmol, 92% yield). LCMS (high pH A): Rt=1.34 min, MH⁺=796.2, 798.2

### Example AR160

### 2-Chloro-4-((2S,5R)-4-(6-(4-(4-(4-(2,4-dioxotetrahydropyrimidin-1(2H)-yl)-1H-pyrrolo[2,3-c]pyridin-1-yl)piperidin-1-yl)butoxy)nicotinoyl)-2,5-dimethylpiperazin-1-yl)benzonitrile (EAR160)

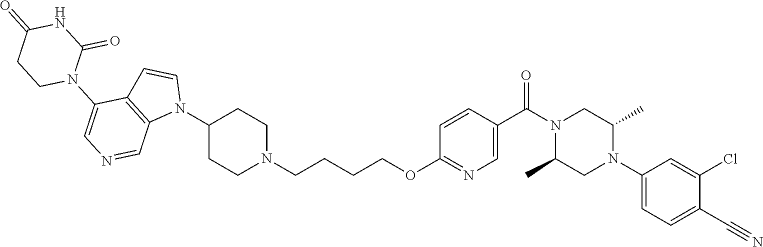 [SMILES: C[C@@H]1CN(c2ccc(C#N)c(Cl)c2)[C@@H](C)CN1C(=O)c1ccc(OCCCCN2CCC(n3ccc4c(N5CCC(=O)NC5=O)cncc43)CC2)nc1]

A mixture of 1-(1-(piperidin-4-yl)-1H-pyrrolo[2,3-c]pyridin-4-yl)dihydropyrimidine-2,4(1H,3H)-dione (may be prepared as described in Example ECB6; 30 mg, 0.096 mmol) and 2-chloro-4-((2S,5R)-2,5-dimethyl-4-(6-(4-oxobutoxy)nicotinoyl)piperazin-1-yl)benzonitrile (may be prepared as described in Description 249; 65 mg, 0.147 mmol) was stirred in DCM (3 mL) at room temperature for 0.5 h. Sodium triacetoxyborohydride (65 mg, 0.307 mmol) was added, and the reaction was stirred at room temperature for 16 h. The mixture was partitioned between DCM (10 mL) and saturated sodium hydrogen carbonate (10 mL), and the organic phase was separated. The aqueous phase was extracted with DCM (5 mL×2). The combined organic phases were washed with water (25 mL) and brine (25 mL), dried, evaporated and purified by MDAP (high pH) to give 2-chloro-4-((2S,5R)-4-(6-(4-(4-(4-(2,4-dioxotetrahydropyrimidin-1(2H)-yl)-1H-pyrrolo[2,3-c]pyridin-1-yl)piperidin-1-yl)butoxy)nicotinoyl)-2,5-dimethylpiperazin-1-yl)benzonitrile (23 mg, 0.031 mmol, 32.5% yield). LCMS (high pH A): Rt=1.07 min, MH⁺=738.3, 740.2

### Example AR161

### N-((1r,4r)-4-(3-Chloro-4-cyanophenoxy)cyclohexyl)-6-(4-((4-(4-(2,4-dioxotetrahydropyrimidin-1(2H)-yl)-6-(trifluoromethyl)-1H-indazol-1-yl)piperidin-1-yl)methyl)piperidin-1-yl)pyridazine-3-carboxamide (EAR161)

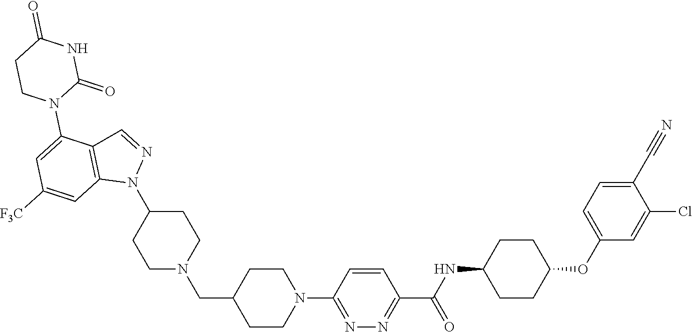 [SMILES: N#Cc1ccc(O[C@H]2CC[C@H](NC(=O)c3ccc(N4CCC(CN5CCC(n6ncc7c(N8CCC(=O)NC8=O)cc(C(F)(F)F)cc76)CC5)CC4)nn3)CC2)cc1Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 58.9 mg, 0.126 mmol) and 1-(1-(piperidin-4-yl)-6-(trifluoromethyl)-1H-indazol-4-yl)dihydropyrimidine-2,4(1H,3H)-dione (may be prepared as described in Example ECB30; 40 mg, 0.105 mmol) were dissolved in DCM (2 ml) and stirred for 10 minutes. Sodium triacetoxyborohydride (66.7 mg, 0.315 mmol) was added, and the mixture was stirred for 1 h, quenched with saturated sodium bicarbonate solution and stirred for overnight (approximately 16 h). The reaction was diluted with DCM, washed with NaHCO₃ and brine, passed through a hydrophobic frit, concentrated and purified by MDAP (high pH) to give N-((1r,4r)-4-(3-chloro-4-cyanophenoxy)cyclohexyl)-6-(4-((4-(4-(2,4-dioxotetrahydropyrimidin-1(2H)-yl)-6-(trifluoromethyl)-1H-indazol-1-yl)piperidin-1-yl)methyl)piperidin-1-yl)pyridazine-3-carboxamide (60 mg, 0.068 mmol, 65.2% yield). LCMS (high pH A): Rt=1.35 min, MH⁺=833.2, 835.2

### Examples AR162 and AR163

### N-((1r,4r)-4-(3-Chloro-4-cyanophenoxy)cyclohexyl)-6-(4-((4-(4-(2,4-dioxotetrahydropyrimidin-1(2H)-yl)-1H-indol-1-yl)-3,3-difluoropiperidin-1-yl)methyl)piperidin-1-yl)pyridazine-3-carboxamide, stereoisomer 1 (EAR162) and

### N-((1r,4r)-4-(3-Chloro-4-cyanophenoxy)cyclohexyl)-6-(4-((4-(4-(2,4-dioxotetrahydropyrimidin-1(2H)-yl)-1H-indol-1-yl)-3,3-difluoropiperidin-1-yl)methyl)piperidin-1-yl)pyridazine-3-carboxamide, stereoisomer 2 (EAR163)

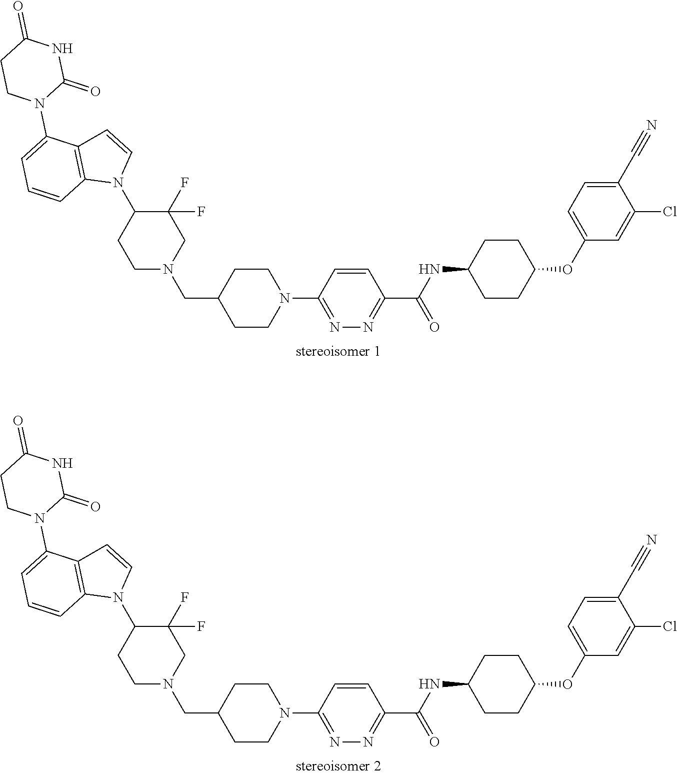 [SMILES: N#Cc1ccc(O[C@H]2CC[C@H](NC(=O)c3ccc(N4CCC(CN5CCC(n6ccc7c(N8CCC(=O)NC8=O)cccc76)C(F)(F)C5)CC4)nn3)CC2)cc1Cl ; N#Cc1ccc(O[C@H]2CC[C@H](NC(=O)c3ccc(N4CCC(CN5CCC(n6ccc7c(N8CCC(=O)NC8=O)cccc76)C(F)(F)C5)CC4)nn3)CC2)cc1Cl]

An enantiomeric mixture of N-((1r,4r)-4-(3-chloro-4-cyanophenoxy)cyclohexyl)-6-(4-((4-(4-(2,4-dioxotetrahydropyrimidin-1(2H)-yl)-1H-indol-1-yl)-3,3-difluoropiperidin-1-yl)methyl)piperidin-1-yl)pyridazine-3-carboxamide (may be prepared as described in Example EAR30; 52 mg, 0.065 mmol) was chirally purified through a Chiralpak IB (250×30 mm, 5 μm) column, eluting with 75:25% MeOH:MeCN (with 0.2% isopropylamine) to give two separate stereoisomers. The first-eluting isomer was further purified through an XBridge Prep C18 (150×30 mm, 5 μm) column, eluting with 50-100% MeOH in (0.1% formic acid in water) to give N-((1r,4r)-4-(3-chloro-4-cyanophenoxy)cyclohexyl)-6-(4-((4-(4-(2,4-dioxotetrahydropyrimidin-1(2H)-yl)-1H-indol-1-yl)-3,3-difluoropiperidin-1-yl)methyl)piperidin-1-yl)pyridazine-3-carboxamide, stereoisomer 1 (18.5 mg, 0.023 mmol, 35.3% yield). LCMS (high pH A): Rt=1.30 min, MH⁺=800.2, 802.2. The second-eluting isomer was further purified through an XBridge Prep C18 (150×30 mm, 5 μm) column, eluting with 50-100% MeOH in (0.1% formic acid in water) to give N-((1r,4r)-4-(3-chloro-4-cyanophenoxy)cyclohexyl)-6-(4-((4-(4-(2,4-dioxotetrahydropyrimidin-1(2H)-yl)-1H-indol-1-yl)-3,3-difluoropiperidin-1-yl)methyl)piperidin-1-yl)pyridazine-3-carboxamide, stereoisomer 2 (22 mg, 0.028 mmol, 42.3% yield). LCMS (high pH A): Rt=1.30 min, MH⁺=800.2, 802.2

### Example AR164

### 2-Chloro-4-((2S,5R)-4-(6-((5-(4-(4-(2,4-dioxotetrahydropyrimidin-1(2H)-yl)-1H-pyrazolo[3,4-c]pyridin-1-yl)piperidin-1-yl)pentyl)oxy)nicotinoyl)-2,5-dimethylpiperazin-1-yl)benzonitrile (EAR164)

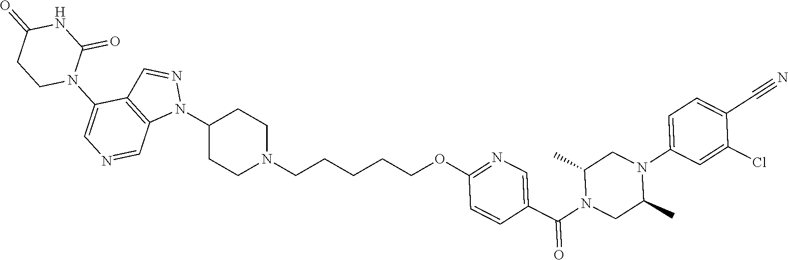 [SMILES: C[C@@H]1CN(c2ccc(C#N)c(Cl)c2)[C@@H](C)CN1C(=O)c1ccc(OCCCCCN2CCC(n3ncc4c(N5CCC(=O)NC5=O)cncc43)CC2)nc1]

A mixture of 1-(1-(piperidin-4-yl)-1H-pyrazolo[3,4-c]pyridin-4-yl)dihydropyrimidine-2,4(1H,3H)-dione (may be prepared as described in Example ECB8; 40 mg, 0.127 mmol) and 2-chloro-4-((2S,5R)-2,5-dimethyl-4-(6-((5-oxopentyl)oxy)nicotinoyl)piperazin-1-yl)benzonitrile (may be prepared as described in Description 253; 80 mg, 0.176 mmol) was stirred in DCM (3 mL) at room temperature for 0.5 h. Sodium triacetoxyborohydride (83 mg, 0.392 mmol) was added, and the reaction was stirred at room temperature for 16 h. The mixture was partitioned between DCM (10 mL) and saturated sodium hydrogen carbonate (10 mL), and the organic phase was separated. The aqueous phase was extracted with DCM (5 mL×2). The combined organic phases were washed with water (25 mL) and brine (25 mL), dried, evaporated and purified by MDAP (formic acid) to give 2-chloro-4-((2S,5R)-4-(6-((5-(4-(4-(2,4-dioxotetrahydropyrimidin-1(2H)-yl)-1H-pyrazolo[3,4-c]pyridin-1-yl)piperidin-1-yl)pentyl)oxy)nicotinoyl)-2,5-dimethylpiperazin-1-yl)benzonitrile 47.8 mg, 0.063 mmol, 49.9% yield). LCMS (high pH A): Rt=1.10 min, MH⁺=753.3

### Example AR165

### 2-Chloro-4-((2S,5R)-4-(6-((5-(4-(4-(2,4-dioxotetrahydropyrimidin-1(2H)-yl)-1H-pyrrolo[2,3-c]pyridin-1-yl)piperidin-1-yl)pentyl)oxy)nicotinoyl)-2,5-dimethylpiperazin-1-yl)benzonitrile (AR165)

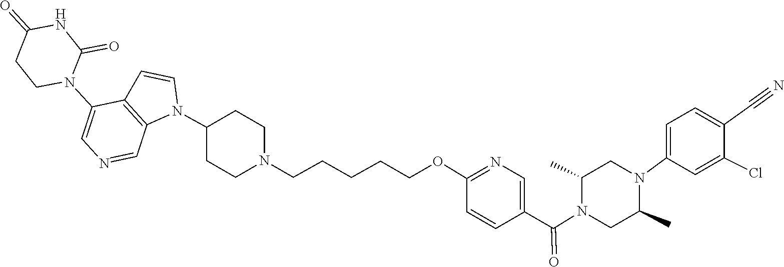 [SMILES: C[C@@H]1CN(c2ccc(C#N)c(Cl)c2)[C@@H](C)CN1C(=O)c1ccc(OCCCCCN2CCC(n3ccc4c(N5CCC(=O)NC5=O)cncc43)CC2)nc1]

A mixture of 1-(1-(piperidin-4-yl)-1H-pyrrolo[2,3-c]pyridin-4-yl)dihydropyrimidine-2,4(1H,3H)-dione (may be prepared as described in Example ECB6; 40 mg, 0.128 mmol) and 2-chloro-4-((2S,5R)-2,5-dimethyl-4-(6-((5-oxopentyl)oxy)nicotinoyl)piperazin-1-yl)benzonitrile (may be prepared as described in Description 253; 80 mg, 0.176 mmol) was stirred in DCM (3 mL) at room temperature for 0.5 h. Sodium triacetoxyborohydride (81 mg, 0.383 mmol) was added, and the reaction was stirred at room temperature for 16 h. The mixture was partitioned between DCM (10 mL) and saturated sodium hydrogen carbonate (10 mL), and the organic phase was separated. The aqueous phase was extracted with DCM (5 mL×2). The combined organic phases were washed with water (25 mL) and brine (25 mL), dried, evaporated and purified by MDAP (high pH) to give 2-chloro-4-((2S,5R)-4-(6-((5-(4-(4-(2,4-dioxotetrahydropyrimidin-1(2H)-yl)-1H-pyrrolo[2,3-c]pyridin-1-yl)piperidin-1-yl)pentyl)oxy)nicotinoyl)-2,5-dimethylpiperazin-1-yl)benzonitrile (32.4 mg, 0.043 mmol, 33.7% yield). LCMS (high pH A): Rt=1.11 min, MH⁺=752.2, 754.2

### Example AR166

### N-((1r,4r)-4-(3-Chloro-4-cyanophenoxy)cyclohexyl)-6-(1-(2-(4-(4-(2,4-dioxotetrahydropyrimidin-1(2H)-yl)-1H-indol-1-yl)piperidin-1-yl)-2-oxoethyl)piperidin-4-yl)pyridazine-3-carboxamide (EAR166)

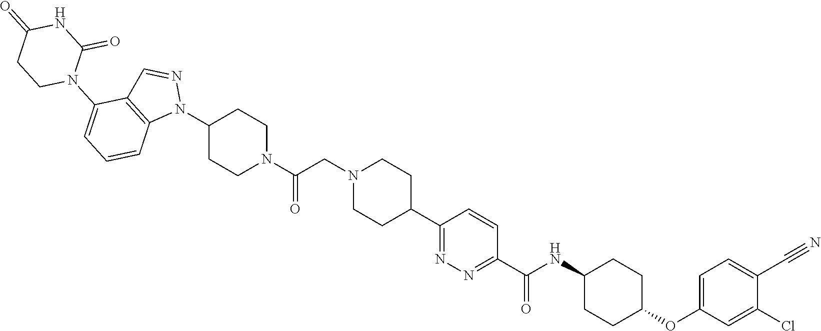 [SMILES: N#Cc1ccc(O[C@H]2CC[C@H](NC(=O)c3ccc(C4CCN(CC(=O)N5CCC(n6ncc7c(N8CCC(=O)NC8=O)cccc76)CC5)CC4)nn3)CC2)cc1Cl]

To 2-(4-(6-(((1r,4r)-4-(3-chloro-4-cyanophenoxy)cyclohexyl)carbamoyl)pyridazin-3-yl)piperidin-1-yl) acetic acid, trifluoroacetic acid salt (may be prepared as described in Description 260; 255 mg, 0.237 mmol) and 1-(1-(piperidin-4-yl)-1H-indol-4-yl)dihydropyrimidine-2,4(1H,3H)-dione (may be prepared as described in Example ECB1; 74.2 mg, 0.237 mmol; used substoichiometrically as the starting acid was approx 50% pure) and HATU (108 mg, 0.285 mmol) in DMF (3 mL) was added DIPEA (0.166 mL, 0.950 mmol). After 18 h, additional portions of HATU (108 mg, 0.285 mmol) and DIPEA (0.166 mL, 0.950 mmol) were added. After 1 h, the solution was purified directly by MDAP (high pH) to give N-((1r,4r)-4-(3-chloro-4-cyanophenoxy)cyclohexyl)-6-(1-(2-(4-(4-(2,4-dioxotetrahydropyrimidin-1(2H)-yl)-1H-indol-1-yl)piperidin-1-yl)-2-oxoethyl)piperidin-4-yl)pyridazine-3-carboxamide (45.6 mg, 0.058 mmol, 24.23% yield). LCMS (high pH A): Rt=1.13 min, MH⁺=792.2

### Example AR167

### N-((1r,4r)-4-(3-Chloro-4-cyanophenoxy)cyclohexyl)-6-(1-(2-(4-(4-(2,4-dioxotetrahydropyrimidin-1(2H)-yl)-1H-indazol-1-yl)piperidin-1-yl)-2-oxoethyl)piperidin-4-yl)pyridazine-3-carboxamide (EAR167)

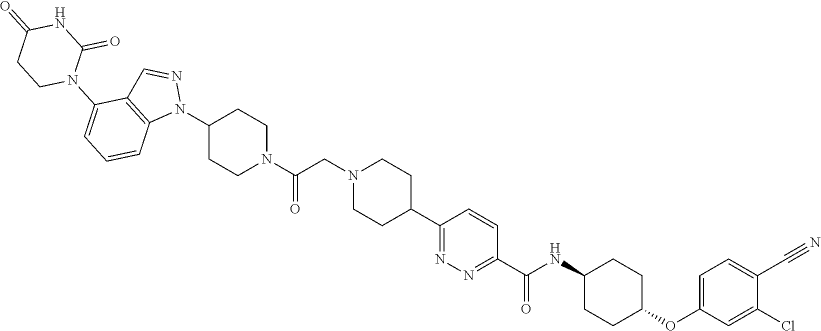 [SMILES: N#Cc1ccc(O[C@H]2CC[C@H](NC(=O)c3ccc(C4CCN(CC(=O)N5CCC(n6ncc7c(N8CCC(=O)NC8=O)cccc76)CC5)CC4)nn3)CC2)cc1Cl]

To 2-(4-(6-(((1r,4r)-4-(3-chloro-4-cyanophenoxy)cyclohexyl)carbamoyl)pyridazin-3-yl)piperidin-1-yl) acetic acid, trifluoroacetic acid salt (may be prepared as described in Description 260; 255 mg, 0.237 mmol) and 1-(1-(piperidin-4-yl)-1H-indazol-4-yl)dihydropyrimidine-2,4(1H,3H)-dione, trifluoroacetic acid salt (may be prepared as described in Example ECB4; 102 mg, 0.237 mmol) and HATU (108 mg, 0.285 mmol) in DMF (3 mL) was added DIPEA (0.166 mL, 0.950 mmol). After 1 h, the solution was purified directly by MDAP (high pH) to give N-((1r,4r)-4-(3-chloro-4-cyanophenoxy)cyclohexyl)-6-(1-(2-(4-(4-(2,4-dioxotetrahydropyrimidin-1(2H)-yl)-1H-indazol-1-yl)piperidin-1-yl)-2-oxoethyl)piperidin-4-yl)pyridazine-3-carboxamide (80.7 mg, 0.102 mmol, 42.8% yield). LCMS (high pH A): Rt=1.09 min, MH⁺=793.2

### Example AR168

### N-((1r,4r)-4-(3-Chloro-4-cyanophenoxy)cyclohexyl)-6-(4-((4-((4-(2,4-dioxotetrahydropyrimidin-1(2H)-yl)-1H-indol-1-yl)methyl)-4-fluoropiperidin-1-yl)methyl)piperidin-1-yl)pyridazine-3-carboxamide (EAR168)

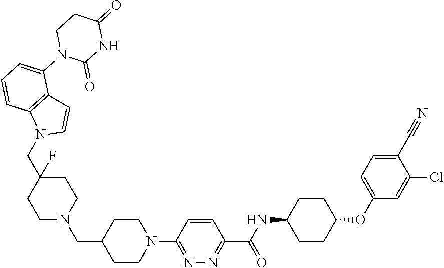 [SMILES: N#Cc1ccc(O[C@H]2CC[C@H](NC(=O)c3ccc(N4CCC(CN5CCC(F)(Cn6ccc7c(N8CCC(=O)NC8=O)cccc76)CC5)CC4)nn3)CC2)cc1Cl]

Sodium triacetoxyborohydride (68 mg, 0.321 mmol) was added to 1-(1-((4-fluoropiperidin-4-yl)methyl)-1H-indol-4-yl)dihydropyrimidine-2,4(1H,3H)-dione (may be prepared as described in Example ECB31; 44 mg, 0.128 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 50 mg, 0.107 mmol) in dichloromethane (5 mL). After 18 hours, saturated sodium bicarbonate solution (5 mL) was added, and the mixture was stirred for 15 minutes. The organic phase was separated, and the aqueous phase was extracted with dichloromethane (2×5 mL). The combined organics were dried, evaporated and purified by MDAP (high pH) to give N-((1r,4r)-4-(3-chloro-4-cyanophenoxy)cyclohexyl)-6-(4-((4-((4-(2,4-dioxotetrahydropyrimidin-1(2H)-yl)-1H-indol-1-yl)methyl)-4-fluoropiperidin-1-yl)methyl)piperidin-1-yl)pyridazine-3-carboxamide (16 mg, 0.020 mmol, 18.80% yield). LCMS (high pH A): Rt=1.27 min, MH⁺=796.2

### Example AR169

### N-((1r,4r)-4-(3-chloro-4-cyanophenoxy)cyclohexyl)-4-(4-((4-(4-(2,4-dioxotetrahydropyrimidin-1(2H)-yl)-1H-indol-1-yl)piperidin-1-yl)methyl)piperidin-1-yl)benzamide (EAR169)

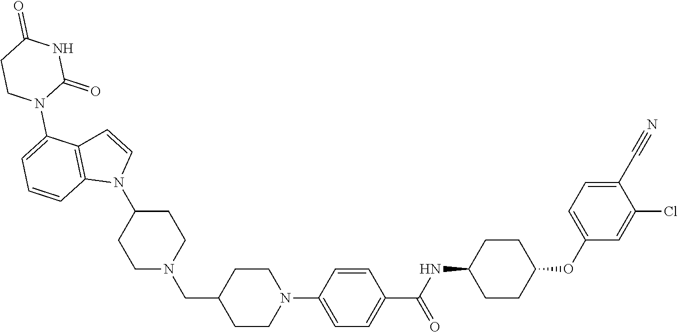 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)cc3)CC2)cc1Cl]

Sodium triacetoxyborohydride (94 mg, 0.444 mmol) was added to N-((1r,4r)-4-(3-chloro-4-cyanophenoxy)cyclohexyl)-4-(4-formylpiperidin-1-yl)benzamide (may be prepared as described in Description 266; 69 mg, 0.148 mmol) and 1-(1-(piperidin-4-yl)-1H-indol-4-yl)dihydropyrimidine-2,4(1H,3H)-dione (may be prepared as described in Example ECB1; 51 mg, 0.163 mmol) in dichloromethane (3 mL). After 18 hours, saturated sodium bicarbonate solution (5 mL) was added, and the mixture was stirred for 15 minutes. The organic phase was separated. The aqueous phase was extracted with dichloromethane (2×5 mL), and the combined organics were dried, evaporated and purified by MDAP (high pH) to give N-((1r,4r)-4-(3-chloro-4-cyanophenoxy)cyclohexyl)-4-(4-((4-(4-(2,4-dioxotetrahydropyrimidin-1(2H)-yl)-1H-indol-1-yl)piperidin-1-yl)methyl)piperidin-1-yl)benzamide (37 mg, 0.049 mmol, 32.8% yield). LCMS (high pH A): Rt=1.31 min, MH⁺=762.2

### Example AR170

### N-((1r,4r)-4-(3-Chloro-4-cyanophenoxy)cyclohexyl)-6-(4-((4-(4-(2,4-dioxotetrahydropyrimidin-1(2H)-yl)-5-fluoro-1H-indol-1-yl)piperidin-1-yl)methyl)piperidin-1-yl)pyridazine-3-carboxamide, hydrochloride (EAR170)

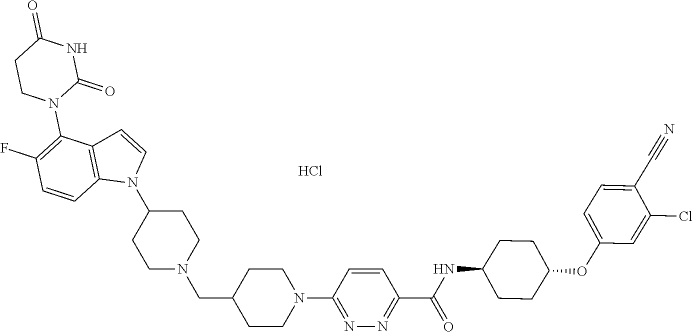 [SMILES: N#Cc1ccc(O[C@H]2CC[C@H](NC(=O)c3ccc(N4CCC(CN5CCC(n6ccc7c(N8CCC(=O)NC8=O)c(F)ccc76)CC5)CC4)nn3)CC2)cc1Cl ; Cl]

A mixture of 1-(5-fluoro-1-(piperidin-4-yl)-1H-indol-4-yl)dihydropyrimidine-2,4(1H,3H)-dione (may be prepared as described in Example ECB32; 30 mg, 0.091 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 42 mg, 0.090 mmol) in DCM (1.0 mL) was stirred in a stoppered vessel at room temperature for 10 min. Sodium triacetoxyborohydride (57 mg, 0.269 mmol) was added, and after 3 h the reaction was washed sequentially with saturated NaHCO3 (aqueous) (2.0 mL) and brine (2.0 mL). The organic layer was passed through a hydrophobic frit, concentrated and purified by MDAP (high pH). This material was dissolved in dioxane (0.5 mL) and treated with 4M HCl in dioxane (0.016 mL, 0.065 mmol). After 2 h, the solvent was evaporated under a stream of nitrogen to give N-((1r,4r)-4-(3-chloro-4-cyanophenoxy)cyclohexyl)-6-(4-((4-(4-(2,4-dioxotetrahydropyrimidin-1(2H)-yl)-5-fluoro-1H-indol-1-yl)piperidin-1-yl)methyl)piperidin-1-yl)pyridazine-3-carboxamide, hydrochloride (37 mg, 0.045 mmol). LCMS (high pH A): Rt=1.31 min, MH⁺=782

### Example AR171

### N-((1r,4r)-4-(3-Chloro-4-cyanophenoxy)cyclohexyl)-6-(4-(((R)-3-(4-(2,4-dioxotetrahydropyrimidin-1(2H)-yl)-1H-indol-1-yl) pyrrolidin-1-yl)methyl)piperidin-1-yl)pyridazine-3-carboxamide, hydrochloride (EAR171)

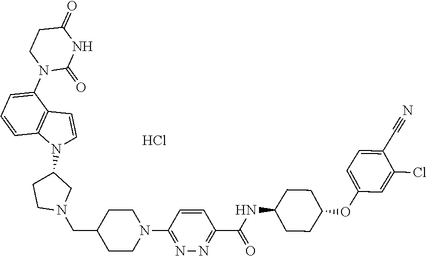 [SMILES: N#Cc1ccc(O[C@H]2CC[C@H](NC(=O)c3ccc(N4CCC(CN5CC[C@H](n6ccc7c(N8CCC(=O)NC8=O)cccc76)C5)CC4)nn3)CC2)cc1Cl ; Cl]

A mixture of (R)-1-(1-(pyrrolidin-3-yl)-1H-indol-4-yl)dihydropyrimidine-2,4(1H,3H)-dione (may be prepared as described in Example ECB33; 43 mg, 0.144 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 82 mg, 0.174 mmol) in DCM (2.06 mL) was stirred at room temperature for 10 min. Sodium triacetoxyborohydride (100 mg, 0.472 mmol) was added, and after 16 h the reaction was diluted with DCM (20 mL), washed sequentially with saturated NaHCO₃ (aqueous) (15 mL) and brine (5 mL). The aqueous layers were extracted with DCM (10 mL). The organic layers were combined, passed through a hydrophobic frit, concentrated and purified by MDAP (high pH). This material was dissolved in dioxane (0.5 mL) and treated with 4M HCl in dioxane (0.358 mL, 0.143 mmol). After 100 min, the solvent was evaporated under a stream of nitrogen to give N-((1r,4r)-4-(3-chloro-4-cyanophenoxy)cyclohexyl)-6-(4-(((R)-3-(4-(2,4-dioxotetrahydropyrimidin-1(2H)-yl)-1H-indol-1-yl) pyrrolidin-1-yl)methyl)piperidin-1-yl)pyridazine-3-carboxamide, hydrochloride (48.6 mg, 0.062 mmol, 42.9% yield). LCMS (high pH A): Rt=1.31 min, MH⁺=750.3, 752.2

### Example AR172

### N-((1r,4r)-4-(3-Chloro-4-cyanophenoxy)cyclohexyl)-6-(4-(((S)-3-(4-(2,4-dioxotetrahydropyrimidin-1(2H)-yl)-1H-indol-1-yl) pyrrolidin-1-yl)methyl)piperidin-1-yl)pyridazine-3-carboxamide, hydrochloride (EAR172)

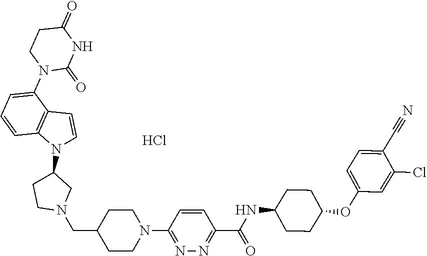 [SMILES: N#Cc1ccc(O[C@H]2CC[C@H](NC(=O)c3ccc(N4CCC(CN5CC[C@@H](n6ccc7c(N8CCC(=O)NC8=O)cccc76)C5)CC4)nn3)CC2)cc1Cl ; Cl]

A mixture of(S)-1-(1-(pyrrolidin-3-yl)-1H-indol-4-yl)dihydropyrimidine-2,4(1H,3H)-dione (may be prepared as described in Example ECB34; 43 mg, 0.144 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 82 mg, 0.174 mmol) in DCM (2.06 mL) was stirred at room temperature for 10 min. Sodium triacetoxyborohydride (100 mg, 0.472 mmol) was added, and after 16 h the reaction was diluted with DCM (20 mL), washed sequentially with saturated NaHCO₃ (aqueous) (15 mL) and brine (5 mL). The aqueous layers were extracted with DCM (10 mL). The organic layers were combined, passed through a hydrophobic frit, concentrated and purified by MDAP (high pH). This material was dissolved in dioxane (0.5 mL) and treated with 4M HCl in dioxane (0.375 mL, 0.15 mmol). After 100 min, the solvent was evaporated under a stream of nitrogen to give N-((1r,4r)-4-(3-chloro-4-cyanophenoxy)cyclohexyl)-6-(4-(((S)-3-(4-(2,4-dioxotetrahydropyrimidin-1(2H)-yl)-1H-indol-1-yl) pyrrolidin-1-yl)methyl)piperidin-1-yl)pyridazine-3-carboxamide hydrochloride (50.2 mg, 0.064 mmol, 44.3% yield). LCMS (high pH A): Rt=1.31 min, MH⁺=750.2, 752.2

### Example AR173

### N-((1r,4r)-4-(3-Chloro-4-cyanophenoxy)cyclohexyl)-6-(4-((6-(4-(2,4-dioxotetrahydropyrimidin-1(2H)-yl)-1H-indol-1-yl)-2-azaspiro[3.3]heptan-2-yl)methyl)piperidin-1-yl)pyridazine-3-carboxamide (EAR173)

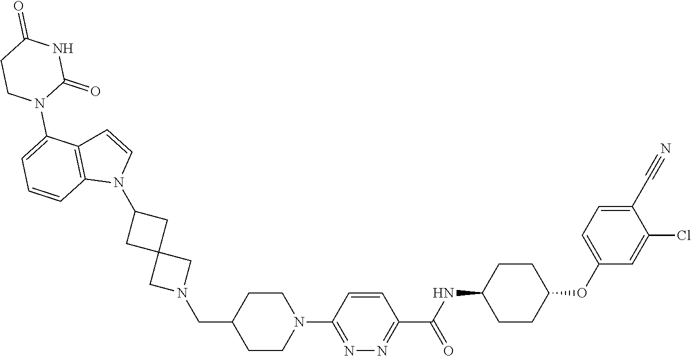 [SMILES: N#Cc1ccc(O[C@H]2CC[C@H](NC(=O)c3ccc(N4CCC(CN5CC6(CC(n7ccc8c(N9CCC(=O)NC9=O)cccc87)C6)C5)CC4)nn3)CC2)cc1Cl]

A mixture of 1-(1-(2-azaspiro[3.3]heptan-6-yl)-1H-indol-4-yl)dihydropyrimidine-2,4(1H,3H)-dione (may be prepared as described in Example ECB35; 80.6 mg, 0.124 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 70.3 mg, 0.150 mmol) in DCM (2.5 mL) was stirred at room temperature for 10 min. Sodium triacetoxyborohydride (79 mg, 0.373 mmol) was added, and after 16 h the reaction was diluted with DCM (20 mL), washed sequentially with saturated NaHCO₃ (aqueous) (15 mL) and brine (5 mL). The aqueous layers were extracted with DCM (10 mL). The organic layers were combined, passed through a hydrophobic frit, concentrated and purified by MDAP (high pH). This material was dissolved in dioxane (0.5 mL) and treated with 4M HCl in dioxane (0.023 mL, 0.09 mmol). After 100 min, the solvent was evaporated under a stream of nitrogen to give a product that was contaminated by impurity. This material was dissolved in DMSO:methanol 3:1 (1 mL) and purified by MDAP (high pH) to give N-((1r,4r)-4-(3-chloro-4-cyanophenoxy)cyclohexyl)-6-(4-((6-(4-(2,4-dioxotetrahydropyrimidin-1(2H)-yl)-1H-indol-1-yl)-2-azaspiro[3.3]heptan-2-yl)methyl)piperidin-1-yl)pyridazine-3-carboxamide (21 mg, 0.027 mmol, 21.77% yield). LCMS (high pH A): Rt=1.25 min, MH⁺=776.3, 778.2

### Example AR174

### 2-Chloro-4-((2S,5R)-4-(6-(2-(4-(4-(2,4-dioxotetrahydropyrimidin-1(2H)-yl)-1H-pyrazolo[3,4-c]pyridin-1-yl)piperidin-1-yl)ethoxy)nicotinoyl)-2,5-dimethylpiperazin-1-yl)benzonitrile (EAR174)

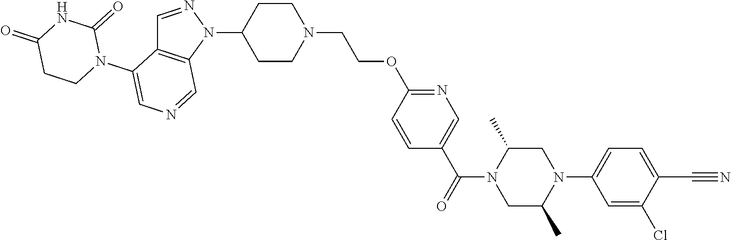 [SMILES: C[C@@H]1CN(c2ccc(C#N)c(Cl)c2)[C@@H](C)CN1C(=O)c1ccc(OCCN2CCC(n3ncc4c(N5CCC(=O)NC5=O)cncc43)CC2)nc1]

A mixture of 1-(1-(piperidin-4-yl)-1H-pyrazolo[3,4-c]pyridin-4-yl)dihydropyrimidine-2,4(1H,3H)-dione (may be prepared as described in Example ECB8; 28 mg, 0.089 mmol) and 2-chloro-4-((2S,5R)-2,5-dimethyl-4-(6-(2-oxoethoxy)nicotinoyl)piperazin-1-yl)benzonitrile (may be prepared as described in Description 276; 37 mg, 0.090 mmol) was stirred in DCM (3 mL) at room temperature for 0.5 h. Sodium triacetoxyborohydride (57 mg, 0.269 mmol) was added, and the reaction was stirred at room temperature for 19 h. The mixture was partitioned between DCM (10 mL) and saturated sodium hydrogen carbonate (10 mL), and the organic phase was separated. The aqueous phase was extracted with DCM (5 mL×2). The combined organic phases were washed with water (25 mL) and brine (25 mL), dried, evaporated and purified by MDAP (formic acid) to give 2-chloro-4-((2S,5R)-4-(6-(2-(4-(4-(2,4-dioxotetrahydropyrimidin-1(2H)-yl)-1H-pyrazolo[3,4-c]pyridin-1-yl)piperidin-1-yl)ethoxy)nicotinoyl)-2,5-dimethylpiperazin-1-yl)benzonitrile (11.3 mg, 0.016 mmol, 17.84% yield). LCMS (high pH A): Rt=1.02 min, MH⁺=711.2

### Example AR175

### 6-(4-((4-(2-Chloro-4-(2,4-dioxotetrahydropyrimidin-1(2H)-yl)-7H-pyrrolo[2,3-d]pyrimidin-7-yl)piperidin-1-yl)methyl)piperidin-1-yl)-N-((1r,4r)-4-(3-chloro-4-cyanophenoxy)cyclohexyl)pyridazine-3-carboxamide (EAR175)

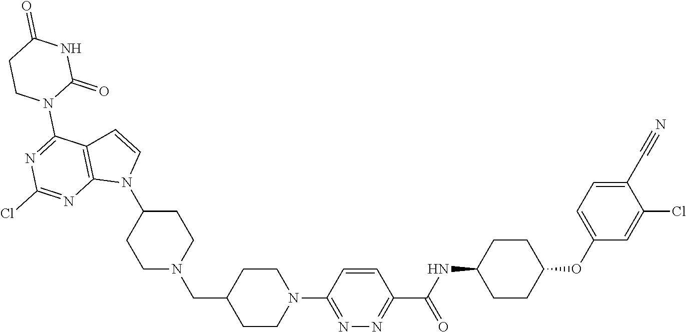 [SMILES: N#Cc1ccc(O[C@H]2CC[C@H](NC(=O)c3ccc(N4CCC(CN5CCC(n6ccc7c(N8CCC(=O)NC8=O)nc(Cl)nc76)CC5)CC4)nn3)CC2)cc1Cl]

A mixture of 1-(2-chloro-7-(piperidin-4-yl)-7H-pyrrolo[2,3-d]pyrimidin-4-yl)dihydropyrimidine-2,4(1H,3H)-dione (may be prepared as described in Example ECB36; 40 mg, 0.115 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 64.4 mg, 0.138 mmol) in DCM (2.0 mL) was stirred in a stoppered vessel at room temperature for 10 min. Sodium triacetoxyborohydride (72.9 mg, 0.344 mmol) was added, and after 64 h the reaction was diluted with DCM and washed sequentially with saturated NaHCO₃ (aqueous) and brine. The organic layer was passed through a hydrophobic frit, concentrated, diluted in 1:1 DMSO:methanol (1 mL) and purified by MDAP (high pH) to give 6-(4-((4-(2-chloro-4-(2,4-dioxotetrahydropyrimidin-1(2H)-yl)-7H-pyrrolo[2,3-d]pyrimidin-7-yl)piperidin-1-yl)methyl)piperidin-1-yl)-N-((1r,4r)-4-(3-chloro-4-cyanophenoxy)cyclohexyl)pyridazine-3-carboxamide (40 mg, 0.047 mmol, 41.4% yield). LCMS (high pH A): Rt=1.35 min, MH⁺=800.2

### Example AR176

### N-((1r,4r)-4-(3-chloro-4-cyanophenoxy)cyclohexyl)-6-(4-((4-(4-(2,4-dioxotetrahydropyrimidin-1(2H)-yl)-6-(trifluoromethyl)-1H-indol-1-yl)piperidin-1-yl)methyl)piperidin-1-yl)pyridazine-3-carboxamide (EAR176)

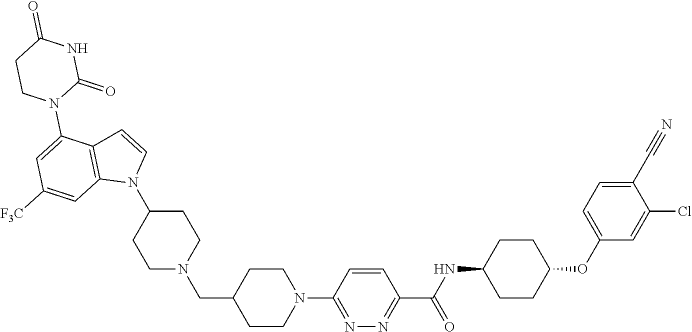 [SMILES: N#Cc1ccc(O[C@H]2CC[C@H](NC(=O)c3ccc(N4CCC(CN5CCC(n6ccc7c(N8CCC(=O)NC8=O)cc(C(F)(F)F)cc76)CC5)CC4)nn3)CC2)cc1Cl]

A mixture of 1-(1-(piperidin-4-yl)-6-(trifluoromethyl)-1H-indol-4-yl)dihydropyrimidine-2,4(1H,3H)-dione (may be prepared as described in Example ECB37; 43.6 mg, 0.115 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 64.4 mg, 0.138 mmol) in DCM (2.0 mL) was stirred in a stoppered vessel at room temperature for 10 min. Sodium triacetoxyborohydride (72.9 mg, 0.344 mmol) was added, and after 64 h the reaction was diluted with DCM and washed sequentially with saturated NaHCO₃ (aqueous) and brine. The organic layer was passed through a hydrophobic frit, concentrated, diluted in 1:1 DMSO:methanol (1 mL) and purified by MDAP (high pH) to give N-((1r,4r)-4-(3-chloro-4-cyanophenoxy)cyclohexyl)-6-(4-((4-(4-(2,4-dioxotetrahydropyrimidin-1(2H)-yl)-6-(trifluoromethyl)-1H-indol-1-yl)piperidin-1-yl)methyl)piperidin-1-yl)pyridazine-3-carboxamide (40 mg, 0.046 mmol, 39.8% yield). LCMS (high pH A): Rt=1.38 min, MH⁺=832.2

### Example AR177

### N-((1r,4r)-4-(3-Chloro-4-cyanophenoxy)cyclohexyl)-6-(4-((4-(8-(2,4-dioxotetrahydropyrimidin-1(2H)-yl) imidazo[1,5-a]pyridin-3-yl)piperidin-1-yl)methyl)piperidin-1-yl)pyridazine-3-carboxamide (EAR177)

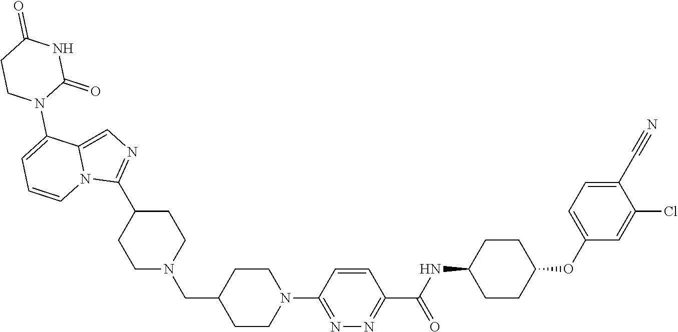 [SMILES: N#Cc1ccc(O[C@H]2CC[C@H](NC(=O)c3ccc(N4CCC(CN5CCC(c6ncc7c(N8CCC(=O)NC8=O)cccn67)CC5)CC4)nn3)CC2)cc1Cl]

A mixture of 1-(3-(piperidin-4-yl) imidazo[1,5-a]pyridin-8-yl)dihydropyrimidine-2,4(1H,3H)-dione (may be prepared as described in Example ECB38; 50 mg, 0.16 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 70 mg, 0.15 mmol) in DCM (2.0 mL) was stirred for 30 min. Sodium triacetoxyborohydride (95 mg, 0.449 mmol) was added, and after 18 h the reaction was diluted with DCM (5 mL) and washed with saturated NaHCO₃ (aqueous) (5 mL). The aqueous phase was extracted with DCM (2×5 mL). The combined organic layers were dried, concentrated, and purified by MDAP (high pH) to give N-((1r,4r)-4-(3-chloro-4-cyanophenoxy)cyclohexyl)-6-(4-((4-(8-(2,4-dioxotetrahydropyrimidin-1(2H)-yl) imidazo[1,5-a]pyridin-3-yl)piperidin-1-yl)methyl)piperidin-1-yl)pyridazine-3-carboxamide (17 mg, 0.022 mmol, 14.85% yield). LCMS (high pH A): Rt=1.14 min, MH⁺=765.3

### Example AR178

### N-((1r,4r)-4-(3-Chloro-4-cyanophenoxy)cyclohexyl)-6-((1-(2-(4-(4-(2,4-dioxotetrahydropyrimidin-1(2H)-yl)-1H-indol-1-yl)piperidin-1-yl) acetyl) azetidin-3-yl)oxy)pyridazine-3-carboxamide (EAR178)

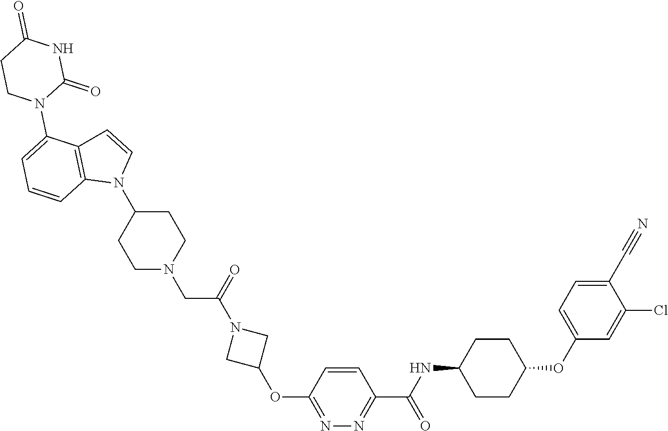 [SMILES: N#Cc1ccc(O[C@H]2CC[C@H](NC(=O)c3ccc(OC4CN(C(=O)CN5CCC(n6ccc7c(N8CCC(=O)NC8=O)cccc76)CC5)C4)nn3)CC2)cc1Cl]

To 2-(4-(4-(2,4-dioxotetrahydropyrimidin-1(2H)-yl)-1H-indol-1-yl)piperidin-1-yl) acetic acid, trifluoroacetate (may be prepared as described in Example ECBL4; 71 mg, 0.088 mmol) and 6-(azetidin-3-yloxy)-N-((1r,4r)-4-(3-chloro-4-cyanophenoxy)cyclohexyl)pyridazine-3-carboxamide (may be prepared as described in Description 289; 38 mg, 0.089 mmol) and HATU (40.5 mg, 0.107 mmol) in DMF (3 mL) was added DIPEA (0.078 mL, 0.444 mmol). After 18 h, the reaction was purified directly by MDAP (high pH) to give N-((1r,4r)-4-(3-chloro-4-cyanophenoxy)cyclohexyl)-6-((1-(2-(4-(4-(2,4-dioxotetrahydropyrimidin-1(2H)-yl)-1H-indol-1-yl)piperidin-1-yl) acetyl) azetidin-3-yl)oxy)pyridazine-3-carboxamide (15.3 mg, 0.020 mmol, 22.08% yield). LCMS (high pH A): Rt=1.11 min, MH⁺=780.2

### Example AR179

### N-((1r,4r)-4-(3-chloro-4-cyanophenoxy)cyclohexyl)-6-(4-((4-(2-(4-(2,4-dioxotetrahydropyrimidin-1(2H)-yl)-1H-indol-1-yl)ethyl)piperidin-1-yl)methyl)piperidin-1-yl)pyridazine-3-carboxamide hydrochloride (AR179)

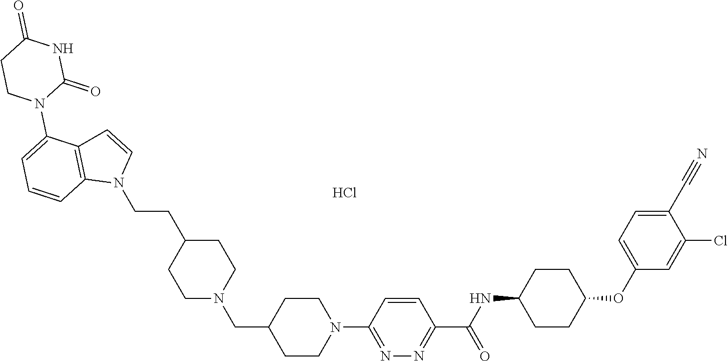 [SMILES: N#Cc1ccc(O[C@H]2CC[C@H](NC(=O)c3ccc(N4CCC(CN5CCC(CCn6ccc7c(N8CCC(=O)NC8=O)cccc76)CC5)CC4)nn3)CC2)cc1Cl ; Cl]

A mixture of 1-(1-(2-(piperidin-4-yl)ethyl)-1H-indol-4-yl)dihydropyrimidine-2,4(1H,3H)-dione (may be prepared as described in Example ECB39; 37.8 mg, 0.111 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 62.4 mg, 0.133 mmol) in DCM (1.5 mL) was stirred for 10 min. Sodium triacetoxyborohydride (70.6 mg, 0.333 mmol) was added, and after 16 h the reaction was diluted with DCM (20 mL) and washed sequentially with saturated NaHCO₃ (aqueous) (15 mL) and brine (5 mL). The aqueous phase was extracted with DCM (10 mL). The combined organic layers were dried, concentrated, and purified by MDAP (high pH) to give material that was taken up in dioxane (0.5 mL) and treated with HCl (4 M in dioxane) (0.011 mL, 0.045 mmol). After 2 h, the reaction was concentrated to give N-((1r,4r)-4-(3-chloro-4-cyanophenoxy)cyclohexyl)-6-(4-((4-(2-(4-(2,4-dioxotetrahydropyrimidin-1(2H)-yl)-1H-indol-1-yl)ethyl)piperidin-1-yl)methyl)piperidin-1-yl)pyridazine-3-carboxamide hydrochloride (14.3 mg, 0.017 mmol, 15.54% yield). LCMS (high pH A): Rt=1.34 min, MH⁺=792.28

### Example AR180

### N-((1r,4r)-4-(3-Chloro-4-cyanophenoxy)cyclohexyl)-4-((4-(4-(2,4-dioxotetrahydropyrimidin-1(2H)-yl)-1H-indol-1-yl)piperidin-1-yl)methyl)benzamide (AR180)

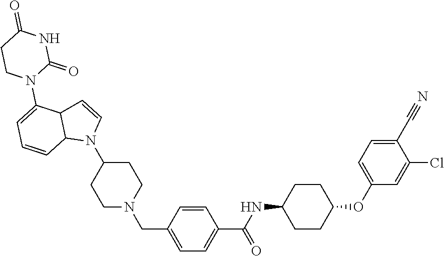 [SMILES: N#Cc1ccc(O[C@H]2CC[C@H](NC(=O)c3ccc(CN4CCC(N5C=CC6C(N7CCC(=O)NC7=O)=CC=CC65)CC4)cc3)CC2)cc1Cl]

A mixture of 1-(1-(piperidin-4-yl)-1H-indol-4-yl)dihydropyrimidine-2,4(1H,3H)-dione (may be prepared as described in Example ECB1; 50 mg, 0.16 mmol) and N-((1r,4r)-4-(3-chloro-4-cyanophenoxy)cyclohexyl)-4-formylbenzamide (may be prepared as described in Description 105; 60 mg, 0.157 mmol) in DCM (5 mL) was stirred for 2 h. Sodium triacetoxyborohydride (91 mg, 0.427 mmol) was added, and after 2 h the reaction was quenched with saturated sodium bicarbonate solution (10 ml), stirred for 20 minutes, and then the organic layer was separated, evaporated in vacuo and purified by MDAP (high pH) to give N-((1r,4r)-4-(3-chloro-4-cyanophenoxy)cyclohexyl)-4-((4-(4-(2,4-dioxotetrahydropyrimidin-1(2H)-yl)-1H-indol-1-yl)piperidin-1-yl)methyl)benzamide (23 mg, 0.034 mmol, 23.77% yield). LCMS (high pH A): Rt=1.22 min, MH⁺=679.2

### Example AR181

### N-((1r,4r)-4-(4-Cyano-3-(trifluoromethyl)phenoxy)cyclohexyl)-5-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyrazine-2-carboxamide (EAR181)

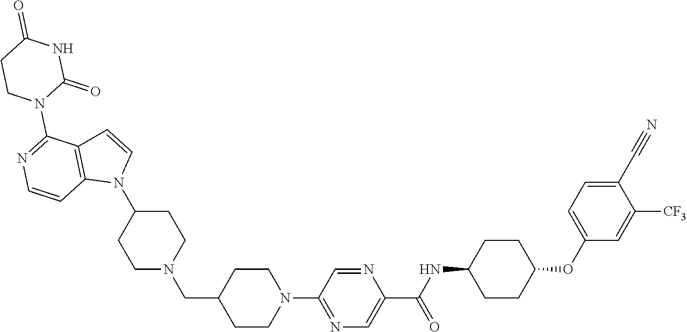 [SMILES: N#Cc1ccc(O[C@H]2CC[C@H](NC(=O)c3cnc(N4CCC(CN5CCC(n6ccc7c(N8CCC(=O)NC8=O)nccc76)CC5)CC4)cn3)CC2)cc1C(F)(F)F]

A mixture of 1-(1-(piperidin-4-yl)-1H-pyrrolo[3,2-c]pyridin-4-yl)dihydropyrimidine-2,4(1H,3H)-dione (may be prepared as described in Example ECB25; 52 mg, 0.166 mmol) and N-((1r,4r)-4-(4-cyano-3-(trifluoromethyl)phenoxy)cyclohexyl)-5-(4-formylpiperidin-1-yl)pyrazine-2-carboxamide (may be prepared as described in Description 98; 92 mg, 0.183 mmol) was stirred in DCM (3 mL). After 45 minutes, sodium triacetoxyborohydride (106 mg, 0.498 mmol) was added, and the mixture was stirred for 2 h, then partitioned between DCM (10 mL) and water (10 mL). The organic phase was separated. The aqueous phase was extracted with DCM (5 ml×2), and the combined organic phases were washed with water (25 mL) and brine (25 mL). The organic phase was dried, concentrated and dissolved in 1:1 methanol:DMSO. A precipitate slowly crystallized and was triturated with diethyl ether. The material was dissolved in DCM, and the solvent was evaporated to give N-((1r,4r)-4-(4-cyano-3-(trifluoromethyl)phenoxy)cyclohexyl)-5-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyrazine-2-carboxamide (62 mg, 0.078 mmol, 46.8% yield). LCMS (high pH A): Rt=1.22 min, MH⁺=799.3

### Example AR182

### N-((1r,4r)-4-(3-Chloro-4-cyanophenoxy)cyclohexyl)-5-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyrazine-2-carboxamide (EAR182)

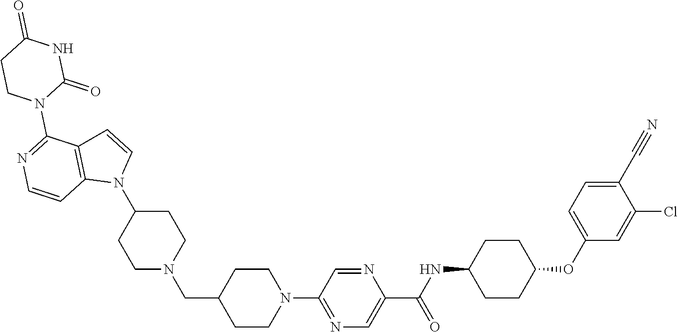 [SMILES: N#Cc1ccc(O[C@H]2CC[C@H](NC(=O)c3cnc(N4CCC(CN5CCC(n6ccc7c(N8CCC(=O)NC8=O)nccc76)CC5)CC4)cn3)CC2)cc1Cl]

A mixture of 1-(1-(piperidin-4-yl)-1H-pyrrolo[3,2-c]pyridin-4-yl)dihydropyrimidine-2,4(1H,3H)-dione (may be prepared as described in Example ECB25; 49.5 mg, 0.158 mmol) and N-((1r,4r)-4-(3-chloro-4-cyanophenoxy)cyclohexyl)-5-(4-formylpiperidin-1-yl)pyrazine-2-carboxamide (may be prepared as described in Description 99; 81 mg, 0.174 mmol) was stirred in DCM (3 mL). After 30 minutes, sodium triacetoxyborohydride (100 mg, 0.474 mmol) was added, and the mixture was stirred for 2.25 h, then partitioned between DCM (10 mL) and water (10 mL). The organic phase was separated. The aqueous phase was extracted with DCM (5 mL×2), and the combined organic phases were washed with water (25 mL) and brine (25 mL). The organic phase was dried, concentrated and purified by MDAP (high pH) to give N-((1r,4r)-4-(3-chloro-4-cyanophenoxy)cyclohexyl)-5-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyrazine-2-carboxamide (56.5 mg, 0.074 mmol, 46.7% yield). LCMS (high pH A): Rt=1.20 min, MH⁺=765.3

### Example AR183

### N-((1r,4r)-4-(3-Chloro-4-cyanophenoxy)cyclohexyl)-6-(4-(((R)-3-(4-(2,4-dioxotetrahydropyrimidin-1(2H)-yl)-1H-indol-1-yl)piperidin-1-yl)methyl)piperidin-1-yl)pyridazine-3-carboxamide hydrochloride (AR183)

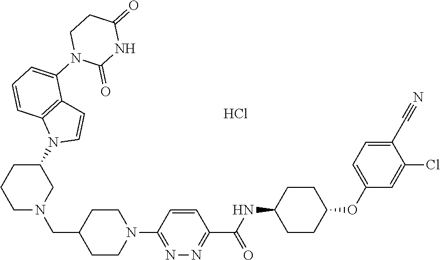 [SMILES: N#Cc1ccc(O[C@H]2CC[C@H](NC(=O)c3ccc(N4CCC(CN5CCC[C@H](n6ccc7c(N8CCC(=O)NC8=O)cccc76)C5)CC4)nn3)CC2)cc1Cl ; Cl]

A mixture of (R)-1-(1-(piperidin-3-yl)-1H-indol-4-yl)dihydropyrimidine-2,4(1H,3H)-dione (may be prepared as described in Example ECB40; 37.8 mg, 0.111 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 97 mg, 0.208 mmol) in DCM (2.5 mL) was stirred for 10 min. Sodium triacetoxyborohydride (120 mg, 0.567 mmol) was added, and after 2 h the reaction was diluted with DCM (20 mL) and washed sequentially with saturated NaHCO₃ (aqueous) (15 mL) and brine (5 mL). The aqueous phase was extracted with DCM (10 mL). The combined organic layers were dried, concentrated, dissolved in DMSO:methanol 3:1 (2 mL) and purified by MDAP (high pH) to give material that was taken up in dioxane (0.5 mL) and treated with HCl (4 M in dioxane) (0.060 mL, 0.238 mmol). After 2 h, the reaction was concentrated to give N-((1r,4r)-4-(3-chloro-4-cyanophenoxy)cyclohexyl)-6-(4-(((R)-3-(4-(2,4-dioxotetrahydropyrimidin-1(2H)-yl)-1H-indol-1-yl)piperidin-1-yl)methyl)piperidin-1-yl)pyridazine-3-carboxamide hydrochloride (77.1 mg, 0.096 mmol, 56.0% yield). LCMS (high pH A): Rt=1.33 min, MH⁺=764.2, 766.1

### Example AR184

### N-((1r,4r)-4-(3-Chloro-4-cyanophenoxy)cyclohexyl)-6-(4-(((S)-3-(4-(2,4-dioxotetrahydropyrimidin-1(2H)-yl)-1H-indol-1-yl)piperidin-1-yl)methyl)piperidin-1-yl)pyridazine-3-carboxamide hydrochloride (EAR184)

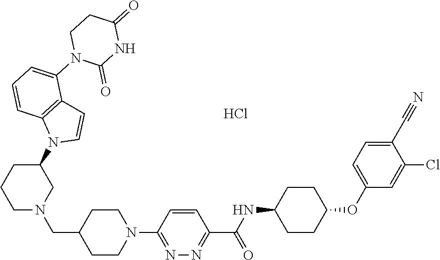 [SMILES: N#Cc1ccc(O[C@H]2CC[C@H](NC(=O)c3ccc(N4CCC(CN5CCC[C@@H](n6ccc7c(N8CCC(=O)NC8=O)cccc76)C5)CC4)nn3)CC2)cc1Cl ; Cl]

A mixture of(S)-1-(1-(piperidin-3-yl)-1H-indol-4-yl)dihydropyrimidine-2,4(1H,3H)-dione (may be prepared as described in Example ECB41; 61.7 mg, 0.198 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 112 mg, 0.239 mmol) in DCM (2.8 mL) was stirred for 10 min. Sodium triacetoxyborohydride (138 mg, 0.652 mmol) was added, and after 2 h the reaction was diluted with DCM (20 mL) and washed sequentially with saturated NaHCO₃ (aqueous) (15 mL) and brine (5 mL). The aqueous phase was extracted with DCM (10 mL). The combined organic layers were dried, concentrated, dissolved in DMSO:methanol 3:1 (2 mL) and purified by MDAP (high pH) to give material that was taken up in dioxane (0.5 mL) and treated with HCl (4 M in dioxane) (0.056 mL, 0.223 mmol). After 2 h, the reaction was concentrated to give N-((1r,4r)-4-(3-chloro-4-cyanophenoxy)cyclohexyl)-6-(4-(((S)-3-(4-(2,4-dioxotetrahydropyrimidin-1(2H)-yl)-1H-indol-1-yl)piperidin-1-yl)methyl)piperidin-1-yl)pyridazine-3-carboxamide hydrochloride (72.6 mg, 0.091 mmol, 45.9% yield). LCMS (high pH A): Rt=1.33 min, MH⁺=764.2, 766.1

### Example AR185

### N-((1r,4r)-4-(3-Chloro-4-cyanophenoxy)cyclohexyl)-6-(4-((2-(4-(2,4-dioxotetrahydropyrimidin-1(2H)-yl)-1H-indol-1-yl)-7-azaspiro[3.5]nonan-7-yl)methyl)piperidin-1-yl)pyridazine-3-carboxamide hydrochloride (EAR185)

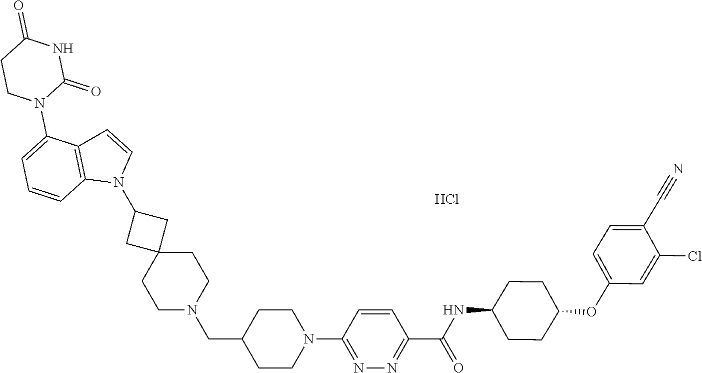 [SMILES: N#Cc1ccc(O[C@H]2CC[C@H](NC(=O)c3ccc(N4CCC(CN5CCC6(CC5)CC(n5ccc7c(N8CCC(=O)NC8=O)cccc75)C6)CC4)nn3)CC2)cc1Cl ; Cl]

A mixture of 1-(1-(7-azaspiro[3.5]nonan-2-yl)-1H-indol-4-yl)dihydropyrimidine-2,4(1H,3H)-dione (may be prepared as described in Example ECB42; 57 mg, 0.137 mmol) and N-((1r,4r)-4-(3-chloro-4-cyanophenoxy)cyclohexyl)-6-(4-formylpiperidin-1-yl)pyridazine-3-carboxamide (may be prepared as described in Description 95; 78 mg, 0.166 mmol) in DCM (2 mL) was stirred for 10 min. Sodium triacetoxyborohydride (87 mg, 0.412 mmol) was added, and after 1.5 h the reaction was diluted with DCM (20 mL) and washed sequentially with saturated NaHCO₃ (aqueous) (15 mL) and brine (5 mL). The aqueous phase was extracted with DCM (10 mL). The combined organic layers were dried, concentrated and dissolved in DMSO:methanol 3:1 (2 mL). Solid began to precipitate, and precipitation increased upon sonication and gentle warming. This solid was collected by filtration and triturated with methanol twice, taken up in dioxane (1 mL) and HCl (4 M in dioxane, 0.085 mL, 0.34 mmol), concentrated, dissolved in DMSO (1 mL) and purified by MDAP (high pH) to give material that was taken up in dioxane (0.5 mL) and treated with HCl (4 M in dioxane, 0.044 mL, 0.176 mmol). After 1 h, the reaction was concentrated to give N-((1r,4r)-4-(3-chloro-4-cyanophenoxy)cyclohexyl)-6-(4-((2-(4-(2,4-dioxotetrahydropyrimidin-1(2H)-yl)-1H-indol-1-yl)-7-azaspiro[3.5]nonan-7-yl)methyl)piperidin-1-yl)pyridazine-3-carboxamide hydrochloride (29.2 mg, 0.035 mmol, 25.3% yield). LCMS (high pH A): Rt=1.35 min, MH⁺=804.2, 806.1

### Example AR186

### N-((1r,4r)-4-(3-Chloro-4-cyanophenoxy)cyclohexyl)-5-(4-((4-(4-(2,4-dioxotetrahydropyrimidin-1(2H)-yl)-1H-indol-1-yl)piperidin-1-yl)methyl)piperidin-1-yl)picolinamide (AR186)

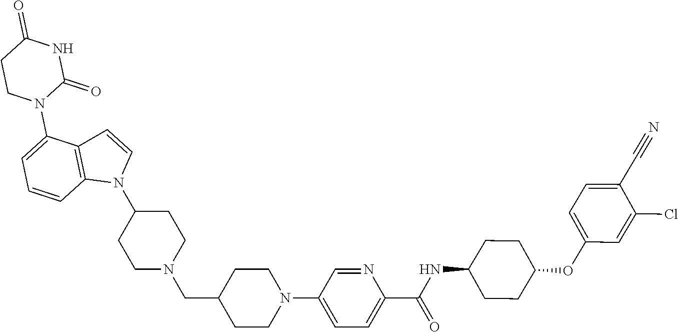 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)cn3)CC2)cc1Cl]

A mixture of 1-(1-(piperidin-4-yl)-1H-indol-4-yl)dihydropyrimidine-2,4(1H,3H)-dione (may be prepared as described in Example ECB1; 53 mg, 0.170 mmol) and N-((1r,4r)-4-(3-chloro-4-cyanophenoxy)cyclohexyl)-5-(4-formylpiperidin-1-yl)picolinamide (may be prepared as described in Description 301; 80 mg, 0.171 mmol) was stirred in DCM (5 mL). After 30 minutes, sodium triacetoxyborohydride (109 mg, 0.514 mmol) was added, and the mixture was stirred for 18 h. Saturated sodium bicarbonate solution (5 mL) was added, and after 15 minutes, the organic phase was separated. The aqueous phase was extracted with dichloromethane (5 mL). The combined organics were dried, concentrated and purified by MDAP (high pH) to give N-((1r,4r)-4-(3-chloro-4-cyanophenoxy)cyclohexyl)-5-(4-((4-(4-(2,4-dioxotetrahydropyrimidin-1(2H)-yl)-1H-indol-1-yl)piperidin-1-yl)methyl)piperidin-1-yl)picolinamide (31 mg, 0.041 mmol, 23.70% yield). LCMS (high pH A): Rt=1.34 min, MH⁺=763.3

### Example AR187

### N-((1r,4r)-4-(3-Chloro-4-cyanophenoxy)cyclohexyl)-4-(4-((4-(4-(2,4-dioxotetrahydropyrimidin-1(2H)-yl)-1H-indol-1-yl)piperidin-1-yl)methyl)piperidin-1-yl)-2-fluorobenzamide (EAR187)

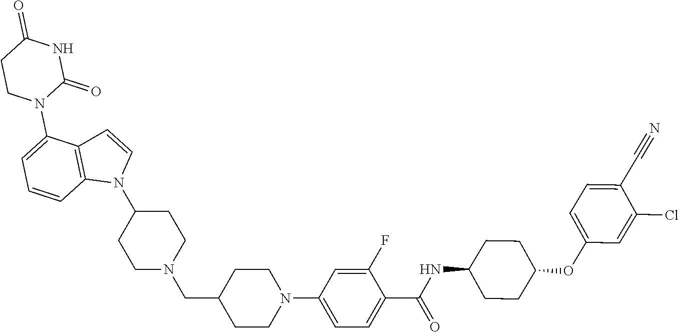 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)cc3F)CC2)cc1Cl]

A mixture of 1-(1-(piperidin-4-yl)-1H-indol-4-yl)dihydropyrimidine-2,4(1H,3H)-dione (may be prepared as described in Example ECB1; 42 mg, 0.134 mmol) and N-((1r,4r)-4-(3-chloro-4-cyanophenoxy)cyclohexyl)-2-fluoro-4-(4-formylpiperidin-1-yl)benzamide (may be prepared as described in Description 304; 80 mg, 0.171 mmol) was stirred in DCM (5 mL). After 30 minutes, sodium triacetoxyborohydride (84 mg, 0.397 mmol) was added, and the mixture was stirred for 18 h. Saturated sodium bicarbonate solution (5 mL) was added, and after 15 minutes, the organic phase was separated. The aqueous phase was extracted with dichloromethane (5 mL). The combined organics were dried, concentrated and purified by MDAP (high pH) to give N-((1r,4r)-4-(3-chloro-4-cyanophenoxy)cyclohexyl)-4-(4-((4-(4-(2,4-dioxotetrahydropyrimidin-1(2H)-yl)-1H-indol-1-yl)piperidin-1-yl)methyl)piperidin-1-yl)-2-fluorobenzamide (28 mg, 0.036 mmol, 27.1% yield). LCMS (high pH A): Rt=1.38 min, MH⁺=780.3

### Example AR188

### N-((1r,4S)-4-(3-Chloro-4-cyanophenoxy)cyclohexyl)-6-(((S)-1-(2-(4-(4-(2,4-dioxotetrahydropyrimidin-1(2H)-yl)-1H-indol-1-yl)piperidin-1-yl) acetyl) pyrrolidin-3-yl)oxy)pyridazine-3-carboxamide (EAR188)

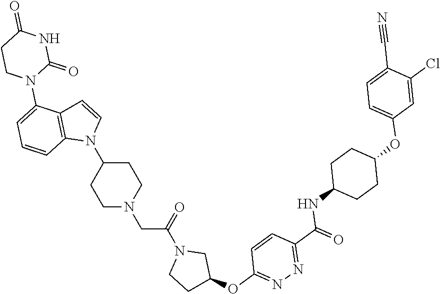 [SMILES: N#Cc1ccc(O[C@H]2CC[C@H](NC(=O)c3ccc(O[C@H]4CCN(C(=O)CN5CCC(n6ccc7c(N8CCC(=O)NC8=O)cccc76)CC5)C4)nn3)CC2)cc1Cl]

To 2-(4-(4-(2,4-dioxotetrahydropyrimidin-1(2H)-yl)-1H-indol-1-yl)piperidin-1-yl) acetic acid trifluoroacetate (may be prepared as described in Example ECBL4; 116 mg, 0.144 mmol), N-((1r,4S)-4-(3-chloro-4-cyanophenoxy)cyclohexyl)-6-(((S)-pyrrolidin-3-yl)oxy)pyridazine-3-carboxamide (may be prepared as described in Description 306; 63.6 mg, 0.144 mmol) and HATU (65.7 mg, 0.173 mmol) in DMF (1 mL) was added DIPEA (0.126 mL, 0.720 mmol). After 1 h the reaction was purified directly by MDAP (high pH) to give N-((1r,4S)-4-(3-chloro-4-cyanophenoxy)cyclohexyl)-6-(((S)-1-(2-(4-(4-(2,4-dioxotetrahydropyrimidin-1(2H)-yl)-1H-indol-1-yl)piperidin-1-yl) acetyl) pyrrolidin-3-yl)oxy)pyridazine-3-carboxamide (18.4 mg, 0.023 mmol, 16.09% yield). LCMS (high pH A): Rt=1.13 min, MH⁺=794.2

### Example AR189

### N-((1r,4r)-4-(4-Cyano-3-(trifluoromethyl)phenoxy)cyclohexyl)-5-(4-((4-(4-(2,4-dioxotetrahydropyrimidin-1(2H)-yl)-5-fluoro-1H-indazol-1-yl)piperidin-1-yl)methyl)piperidin-1-yl)pyrazine-2-carboxamide hydrochloride (EAR189)

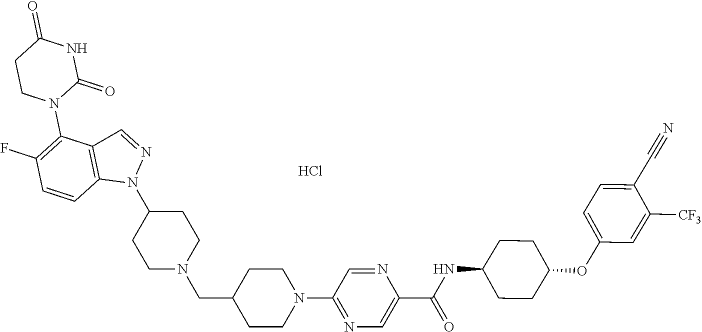 [SMILES: N#Cc1ccc(O[C@H]2CC[C@H](NC(=O)c3cnc(N4CCC(CN5CCC(n6ncc7c(N8CCC(=O)NC8=O)c(F)ccc76)CC5)CC4)cn3)CC2)cc1C(F)(F)F ; Cl]

A mixture of 1-(5-fluoro-1-(piperidin-4-yl)-1H-indazol-4-yl)dihydropyrimidine-2,4(1H,3H)-dione (may be prepared as described in Example ECB21; 70 mg, 0.211 mmol) and N-((1r,4r)-4-(4-cyano-3-(trifluoromethyl)phenoxy)cyclohexyl)-5-(4-formylpiperidin-1-yl)pyrazine-2-carboxamide (may be prepared as described in Description 98; 106 mg, 0.211 mmol) was stirred in DCM (1.5 mL). After 10 minutes, sodium triacetoxyborohydride (67 mg, 0.316 mmol) was added, and the mixture was stirred for 5 h, washed sequentially with sat. NaHCO₃ (aq) (2.0 mL) and brine (2.0 mL), passed through a hydrophobic frit and purified through an XSelect CSH Prep C18 (150×30 mm, 5 μm) column, eluting with 50-100% MeCN in 10 mM NH₄HCO₃ in water adjusted to pH 10 with aqueous NH₃ to give material that was dissolved in DCM (0.25 mL) and 1,4-dioxane (0.25 mL). 4 M HCl in 1,4-dioxane (25 μL, 0.100 mmol) was added, and the suspension was stirred at room temperature in a stoppered vessel for 1 h. The solvent was evaporated under a stream of nitrogen to give N-((1r,4r)-4-(4-cyano-3-(trifluoromethyl)phenoxy)cyclohexyl)-5-(4-((4-(4-(2,4-dioxotetrahydropyrimidin-1(2H)-yl)-5-fluoro-1H-indazol-1-yl)piperidin-1-yl)methyl)piperidin-1-yl)pyrazine-2-carboxamide hydrochloride (52 mg, 0.061 mmol, 28.8% yield). LCMS (high pH A): Rt=1.31 min, MH⁺=817.2

### Example AR190

### N-((1r,4r)-4-(3-Chloro-4-cyanophenoxy)cyclohexyl)-5-(4-((4-(4-(2,4-dioxotetrahydropyrimidin-1(2H)-yl)-5-fluoro-1H-indazol-1-yl)piperidin-1-yl)methyl)piperidin-1-yl)pyrazine-2-carboxamide hydrochloride (EAR190)

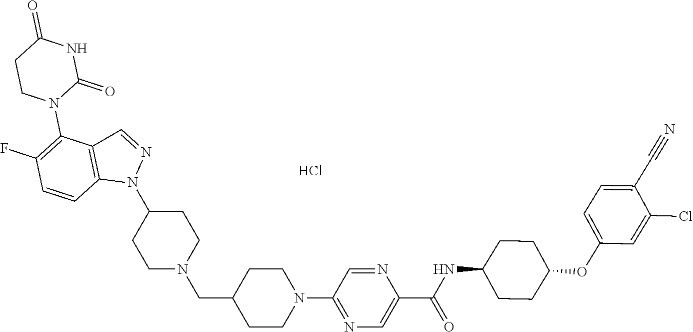 [SMILES: N#Cc1ccc(O[C@H]2CC[C@H](NC(=O)c3cnc(N4CCC(CN5CCC(n6ncc7c(N8CCC(=O)NC8=O)c(F)ccc76)CC5)CC4)cn3)CC2)cc1Cl ; Cl]

A mixture of 1-(5-fluoro-1-(piperidin-4-yl)-1H-indazol-4-yl)dihydropyrimidine-2,4(1H,3H)-dione (may be prepared as described in Example ECB21; 70 mg, 0.211 mmol) and N-((1r,4r)-4-(4-cyano-3-(trifluoromethyl)phenoxy)cyclohexyl)-5-(4-formylpiperidin-1-yl)pyrazine-2-carboxamide (may be prepared as described in Description 98; 99 mg, 0.211 mmol) was stirred in DCM (1.5 mL). After 10 minutes, sodium triacetoxyborohydride (67 mg, 0.316 mmol) was added, and the mixture was stirred for 5 h, washed sequentially with saturated NaHCO₃ (aqueous) (2.0 mL) and brine (2.0 mL), passed through a hydrophobic frit and purified through an XSelect CSH Prep C18 (150×30 mm, 5 μm) column, eluting with 50-100% MeCN in 10 mM NH₄HCO₃ in water adjusted to pH 10 with aqueous NH₃ to give material that was dissolved in DCM (0.25 mL) and 1,4-dioxane (0.25 mL). 4 M HCl in 1,4-dioxane (14 μL, 0.056 mmol) was added, and the suspension was stirred at room temperature in a stoppered vessel for 1 h. The solvent was evaporated under a stream of nitrogen to give N-((1r,4r)-4-(3-chloro-4-cyanophenoxy)cyclohexyl)-5-(4-((4-(4-(2,4-dioxotetrahydropyrimidin-1(2H)-yl)-5-fluoro-1H-indazol-1-yl)piperidin-1-yl)methyl)piperidin-1-yl)pyrazine-2-carboxamide hydrochloride (27 mg, 0.033 mmol, 15.59% yield). LCMS (high pH A): Rt=1.30 min, MH⁺=783

### Examples AR191 and AR191

### N-((1r,4r)-4-(3-Chloro-4-cyanophenoxy)cyclohexyl)-6-(4-((4-(1-(4-(2,4-dioxotetrahydropyrimidin-1(2H)-yl)-1H-indol-1-yl)ethyl)piperidin-1-yl)methyl)piperidin-1-yl)pyridazine-3-carboxamide hydrochloride, stereoisomer 1 (EAR191) and

### N-((1r,4r)-4-(3-Chloro-4-cyanophenoxy)cyclohexyl)-6-(4-((4-(1-(4-(2,4-dioxotetrahydropyrimidin-1(2H)-yl)-1H-indol-1-yl)ethyl)piperidin-1-yl)methyl)piperidin-1-yl)pyridazine-3-carboxamide hydrochloride, stereoisomer 2 (EAR192)

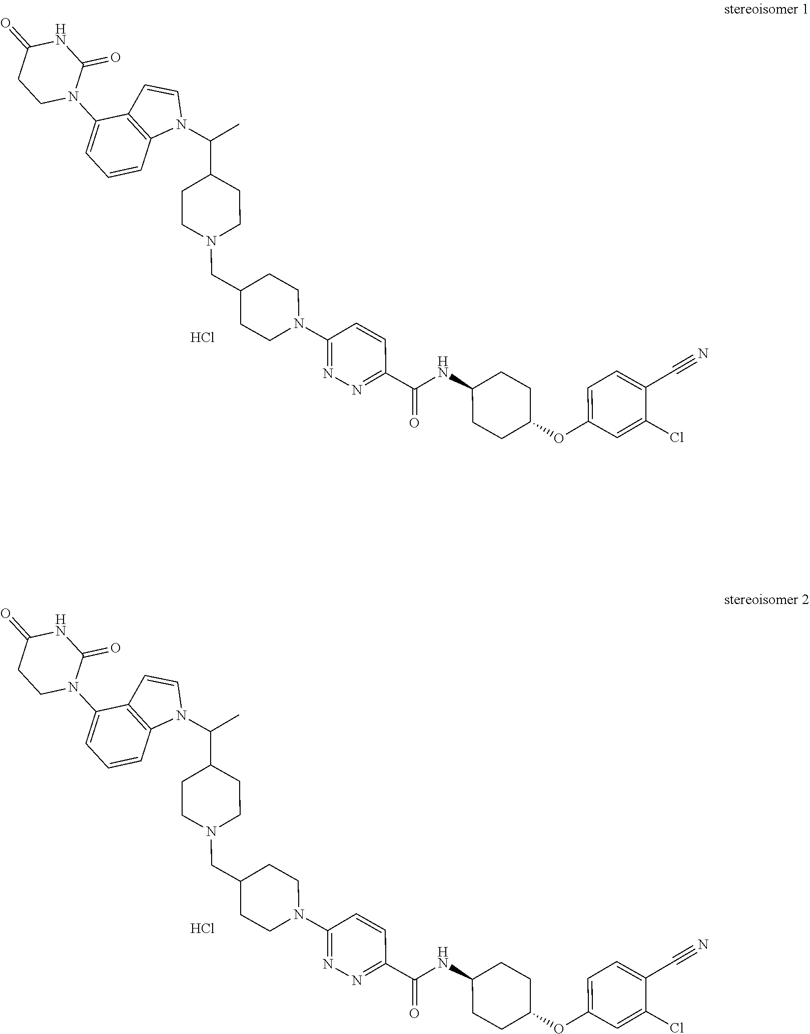 [SMILES: CC(C1CCN(CC2CCN(c3ccc(C(=O)N[C@H]4CC[C@H](Oc5ccc(C#N)c(Cl)c5)CC4)nn3)CC2)CC1)n1ccc2c(N3CCC(=O)NC3=O)cccc21 ; Cl ; CC(C1CCN(CC2CCN(c3ccc(C(=O)N[C@H]4CC[C@H](Oc5ccc(C#N)c(Cl)c5)CC4)nn3)CC2)CC1)n1ccc2c(N3CCC(=O)NC3=O)cccc21 ; Cl]

N-((1r,4r)-4-(3-Chloro-4-cyanophenoxy)cyclohexyl)-6-(4-formylpiperidin-1-yl)pyridazine-3-carboxamide (may be prepared as described in Description 95; 100 mg, 0.213 mmol) and 1-(1-(1-(piperidin-4-yl)ethyl)-1H-indol-4-yl)dihydropyrimidine-2,4(1H,3H)-dione (may be prepared as described in Example CB43; 60 mg, 0.176 mmol) were suspended in DCM (2.5 mL). After 10 min, sodium triacetoxyborohydride (122 mg, 0.576 mmol) was added. After 17 h, the mixture was diluted with DCM (20 mL), washed with NaHCO₃ (15 mL) and brine (5 mL) and the aqueous layers were extracted with DCM (10 mL). The organics were combined, dried by passing through a hydrophobic frit and purified through an XBridge Prep Shield C18 (150×30 mm, 5 μm), eluting with 40-100% MeCN in 10 mM NH₄HCO₃ in water adjusted to pH 10 with aqueous NH₃. This material was chirally separated through a 30 mm×25 cm Chiralpak ID (5 μm) column, eluting with 50% MeCN (+0.2% isopropylamine): 50% MeOH (+0.2% isopropylamine) to give stereoisomers 1 and 2.

To the first-eluting isomer in dioxane (0.5 mL) was added HCl (4 M in dioxane) (0.032 mL, 0.126 mmol). The resulting suspension was stirred at room temperature for 90 min and concentrated to give N-((1r,4r)-4-(3-chloro-4-cyanophenoxy)cyclohexyl)-6-(4-((4-(1-(4-(2,4-dioxotetrahydropyrimidin-1(2H)-yl)-1H-indol-1-yl)ethyl)piperidin-1-yl)methyl)piperidin-1-yl)pyridazine-3-carboxamide hydrochloride, stereoisomer 1 (37 mg, 0.045 mmol, 26.5% yield). LCMS (high pH A): Rt=1.34 min, MH⁺=792.2, 794.2.

To the second-eluting isomer was in dioxane (0.5 mL) was added of HCl (4 M in dioxane) (0.037 mL, 0.148 mmol). The resulting suspension was stirred at room temperature for 90 min and concentrated to give N-((1r,4r)-4-(3-chloro-4-cyanophenoxy)cyclohexyl)-6-(4-((4-(1-(4-(2,4-dioxotetrahydropyrimidin-1(2H)-yl)-1H-indol-1-yl)ethyl)piperidin-1-yl)methyl)piperidin-1-yl)pyridazine-3-carboxamide hydrochloride, stereoisomer 2 (40.8 mg, 0.049 mmol, 29.1% yield). LCMS (high pH A): Rt=1.35 min, MH⁺=792.2, 794.1

### Example AR193

### N-((1r,4r)-4-(3-Chloro-4-cyanophenoxy)cyclohexyl)-6-(2-(4-(4-(2,4-dioxotetrahydropyrimidin-1(2H)-yl)-1H-indol-1-yl)piperidin-1-yl)-7-azaspiro[3.5]nonan-7-yl)pyridazine-3-carboxamide (EAR193)

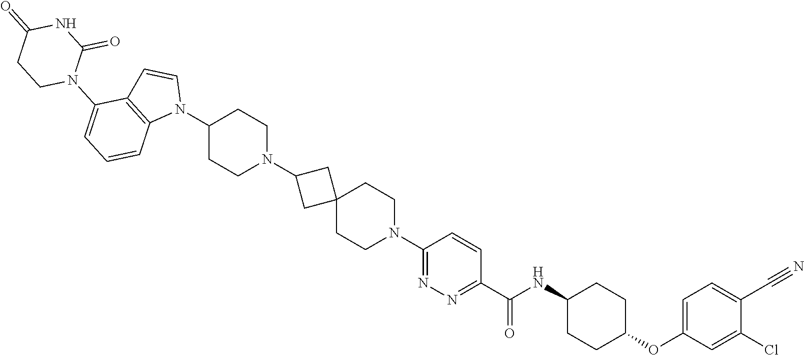 [SMILES: N#Cc1ccc(O[C@H]2CC[C@H](NC(=O)c3ccc(N4CCC5(CC4)CC(N4CCC(n6ccc7c(N8CCC(=O)NC8=O)cccc76)CC4)C5)nn3)CC2)cc1Cl]

A mixture of 1-(1-(piperidin-4-yl)-1H-indol-4-yl)dihydropyrimidine-2,4(1H,3H)-dione (may be prepared as described in Example ECB1; 50 mg, 0.160 mmol) and N-((1r,4r)-4-(3-chloro-4-cyanophenoxy)cyclohexyl)-6-(2-oxo-7-azaspiro[3.5]nonan-7-yl)pyridazine-3-carboxamide (may be prepared as described in Description 310; 100 mg, 0.202 mmol) was stirred in DCM (1 mL). After 2 h, sodium triacetoxyborohydride (70 mg, 0.330 mmol) was added, and the mixture was stirred for 39 h. Saturated sodium bicarbonate solution (2 mL) was added, and after 5 minutes, the organic phase was separated. The aqueous phase was extracted with dichloromethane (2×2 mL). The combined organics were dried, concentrated and purified by MDAP (high pH) to give N-((1r,4r)-4-(3-chloro-4-cyanophenoxy)cyclohexyl)-4-(4-((4-(4-(2,4-dioxotetrahydropyrimidin-1(2H)-yl)-1H-indol-1-yl)piperidin-1-yl)methyl)piperidin-1-yl)-2-fluorobenzamide (28 mg, 0.036 mmol, 27.1% yield). LCMS (high pH A): Rt=1.28 min, MH⁺=790.3

### Example AR194

### N-((1r,4r)-4-(3-Chloro-4-cyanophenoxy)cyclohexyl)-6-(4-((4-(4-(2,4-dioxotetrahydropyrimidin-1(2H)-yl)-1H-indol-1-yl)cyclohexyl)methyl)piperazin-1-yl)pyridazine-3-carboxamide, trifluoroacetic acid salt (EAR194)

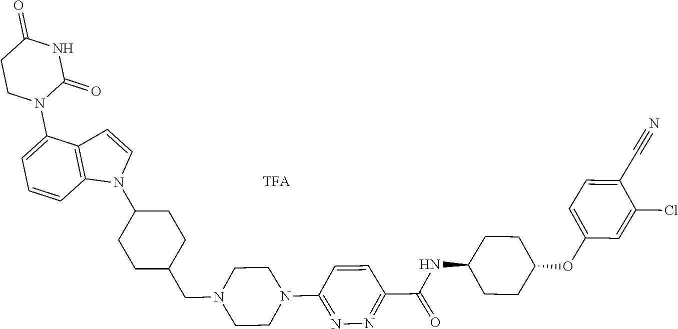 [SMILES: N#Cc1ccc(O[C@H]2CC[C@H](NC(=O)c3ccc(N4CCN(CC5CCC(n6ccc7c(N8CCC(=O)NC8=O)cccc76)CC5)CC4)nn3)CC2)cc1Cl ; "TFA"]

To (1r,4r)-4-(4-(2,4-dioxo-3-((2-(trimethylsilyl)ethoxy)methyl)tetrahydropyrimidin-1(2H)-yl)-1H-indol-1-yl)cyclohexane-1-carbaldehyde (may be prepared as described in Description 315; 98 mg, 0.209 mmol), N-((1r,4r)-4-(3-chloro-4-cyanophenoxy)cyclohexyl)-6-(piperazin-1-yl)pyridazine-3-carboxamide trifluoroacetic acid salt (may be prepared as described in Description 121; 116 mg, 0.209 mmol) in DCM (5 mL) was added acetic acid (0.036 mL, 0.626 mmol). After 15 minutes, sodium triacetoxyborohydride (111 mg, 0.522 mmol) was added, followed by stirring for 6 h, followed by the addition trifluoroacetic acid (0.322 mL, 4.17 mmol). After 2 h, a further portion of trifluoroacetic acid (0.322 mL, 4.17 mmol) was added, stirred overnight, cooled in an ice-water bath and was slowly treated with ammonia (7 M in methanol) (2.98 mL, 20.87 mmol). After 10 mins, the reaction was warmed to room temperature stirred for 2 h, concentrated, dissolved in EtOAc (10 mL) and diluted with saturated aqueous sodium bicarbonate (10 mL). The layers were separated, and the aqueous layer was extracted with EtOAc (10 mL). The combined organic layers were filtered through a hydrophobic, fit, concentrated and purified over a Zorbax SB-Phenyl (150×30 mm, 5 μm) column, eluting with 40-100% MeCN (0.1% TFA) in water (0.1% TFA) to give N-((1r,4r)-4-(3-Chloro-4-cyanophenoxy)cyclohexyl)-6-(4-((4-(4-(2,4-dioxotetrahydropyrimidin-1(2H)-yl)-1H-indol-1-yl)cyclohexyl)methyl)piperazin-1-yl)pyridazine-3-carboxamide, trifluoroacetic acid salt (57 mg, 0.065 mmol, 31% yield). LCMS (high pH A): Rt=1.33 min, MH⁺=764.3, 766.2

### Example AR195

### N-((1r,4r)-4-(3-chloro-4-cyanophenoxy)cyclohexyl)-4-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)benzamide hydrochloride (EAR195)

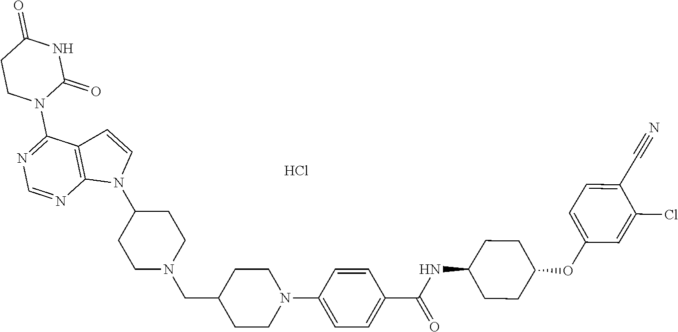 [SMILES: N#Cc1ccc(O[C@H]2CC[C@H](NC(=O)c3ccc(N4CCC(CN5CCC(n6ccc7c(N8CCC(=O)NC8=O)ncnc76)CC5)CC4)cc3)CC2)cc1Cl ; Cl]

N-((1r,4r)-4-(3-Chloro-4-cyanophenoxy)cyclohexyl)-4-(4-formylpiperidin-1-yl)benzamide (may be prepared as described in Description 266; 77 mg, 0.165 mmol) and 1-(7-(piperidin-4-yl)-7H-pyrrolo[2,3-d]pyrimidin-4-yl)dihydropyrimidine-2,4(1H,3H)-dione (may be prepared as described in Example ECB17; 50 mg, 0.159 mmol) were stirred in DCM (3 mL). After 20 min, sodium triacetoxyborohydride (172 mg, 0.812 mmol) was added. After 6 h, the reaction was partitioned between DCM (10 mL) and water (10 mL), and the organic phase was separated. The aqueous phase was extracted with DCM (5 mL×2), and the combined organic phases were washed with water (20 mL) and brine (20 mL), dried, evaporated and was purified by MDAP (high pH) to give residue that was dissolved in 2:1 acetonitrile and water solvent and treated with HCl in ethyl acetate to give N-((1r,4r)-4-(3-chloro-4-cyanophenoxy)cyclohexyl)-4-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)benzamide hydrochloride (40 mg, 0.050 mmol, 31.4% yield). LCMS (high pH A): Rt=1.22 min, MH⁺=764.3

### Example AR196

### N-((1r,4r)-4-(3-Chloro-4-cyanophenoxy)cyclohexyl)-5-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyrazine-2-carboxamide hydrochloride (EAR196)

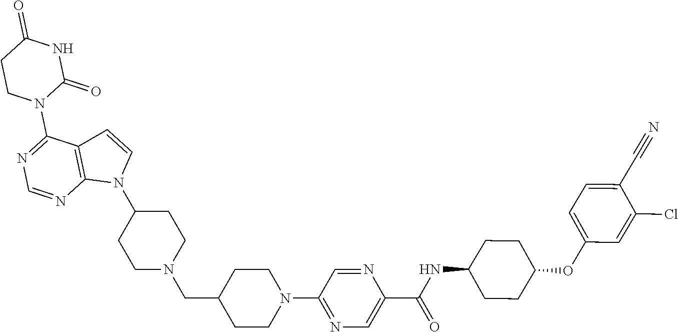 [SMILES: N#Cc1ccc(O[C@H]2CC[C@H](NC(=O)c3cnc(N4CCC(CN5CCC(n6ccc7c(N8CCC(=O)NC8=O)ncnc76)CC5)CC4)cn3)CC2)cc1Cl]

A mixture of 1-(7-(piperidin-4-yl)-7H-pyrrolo[2,3-d]pyrimidin-4-yl)dihydropyrimidine-2,4(1H,3H)-dione (may be prepared as described in Example ECB17; 67.4 mg, 0.214 mmol) and N-((1r,4r)-4-(3-chloro-4-cyanophenoxy)cyclohexyl)-5-(4-formylpiperidin-1-yl)pyrazine-2-carboxamide (may be prepared as described in Description D99; 100 mg, 0.214 mmol) was stirred in DCM (2.14 mL). After 35 minutes, sodium triacetoxyborohydride (91 mg, 0.427 mmol) was added, and the mixture was stirred for 16.5 h, then partitioned between DCM (6 mL), brine (1 mL) and water (8 mL). The organic phase was separated. The aqueous phase was extracted with DCM (9 mL), and the combined organic phases were concentrated and purified by MDAP (high pH) to give residue that was dissolved in 2:1 acetonitrile and water (2 mL), cooled in an ice water bath and treated with HCl (4 M in 1,4-dioxane, 27.9 μL, 1.5 eq). After 35 minutes, the reaction was concentrated to give N-((1r,4r)-4-(3-Chloro-4-cyanophenoxy)cyclohexyl)-5-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyrazine-2-carboxamide hydrochloride (43.9 mg, 0.055 mmol, 25.6% yield). LCMS (high pH A): Rt=1.23 min, MH⁺=766.3

### Example AR197

### N-((1r,4r)-4-(3-Chloro-4-cyanophenoxy)cyclohexyl)-4-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)benzamide (EAR197)

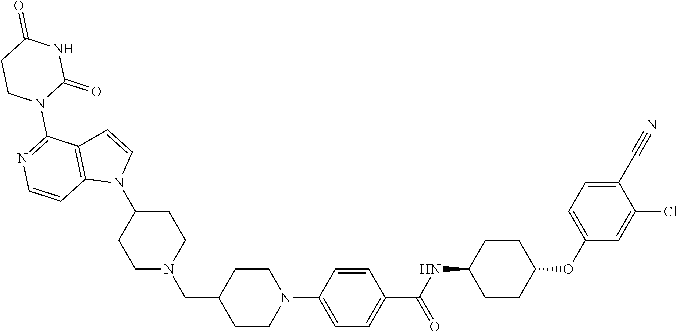 [SMILES: N#Cc1ccc(O[C@H]2CC[C@H](NC(=O)c3ccc(N4CCC(CN5CCC(n6ccc7c(N8CCC(=O)NC8=O)nccc76)CC5)CC4)cc3)CC2)cc1Cl]

N-((1r,4r)-4-(3-Chloro-4-cyanophenoxy)cyclohexyl)-4-(4-formylpiperidin-1-yl)benzamide (may be prepared as described in Description 266; 52 mg, 0.112 mmol) and 1-(1-(piperidin-4-yl)-1H-pyrrolo[3,2-c]pyridin-4-yl)dihydropyrimidine-2,4(1H,3H)-dione (may be prepared as described in Example ECB25; 35 mg, 0.112 mmol) were stirred in DCM (3 mL) for 2 h. Sodium triacetoxyborohydride (71 mg, 0.335 mmol) was added, and the reaction was stirred 1 h. Saturated sodium bicarbonate solution (5 mL) was added, and the mixture was stirred for 10 minutes. The organic phase was separated. The aqueous phase was extracted with dichloromethane (5 mL), and the combined organics were dried, evaporated and purified by MDAP (high pH) to give N-((1r,4r)-4-(3-chloro-4-cyanophenoxy)cyclohexyl)-4-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)benzamide (35 mg, 0.046 mmol, 41.1% yield). LCMS (high pH A): Rt=1.21 min, MH⁺=763.3

### Example AR198

### N-((1r,4r)-4-(4-Cyano-3-(trifluoromethyl)phenoxy)cyclohexyl)-5-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyrazine-2-carboxamide (EAR198)

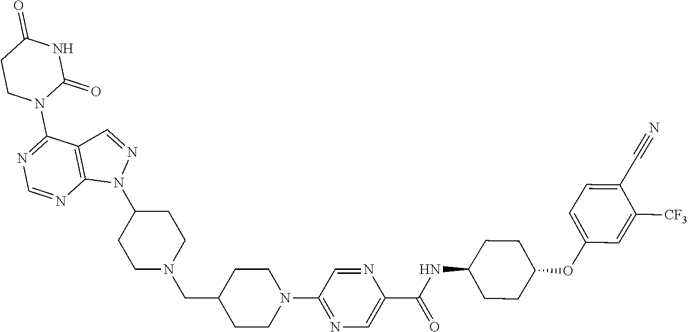 [SMILES: N#Cc1ccc(O[C@H]2CC[C@H](NC(=O)c3cnc(N4CCC(CN5CCC(n6ncc7c(N8CCC(=O)NC8=O)ncnc76)CC5)CC4)cn3)CC2)cc1C(F)(F)F]

A mixture of 1-(7-(piperidin-4-yl)-7H-pyrrolo[2,3-d]pyrimidin-4-yl)dihydropyrimidine-2,4(1H,3H)-dione (may be prepared as described in Example ECB17; 55 mg, 0.175 mmol) and N-((1r,4r)-4-(4-cyano-3-(trifluoromethyl)phenoxy)cyclohexyl)-5-(4-formylpiperidin-1-yl)pyrazine-2-carboxamide (may be prepared as described in Description 98; 92 mg, 0.183 mmol) was stirred in DCM (3 mL). After 20 minutes, sodium triacetoxyborohydride (185 mg, 0.875 mmol) was added, and the mixture was stirred for 6 h, then partitioned between DCM (10 mL) and water (10 mL). The organic phase was separated. The aqueous phase was extracted with DCM (2×5 mL), and the combined organic phases were washed with water (20 mL) and brine (20 mL), concentrated and purified by MDAP (high pH) to give N-((1r,4r)-4-(4-cyano-3-(trifluoromethyl)phenoxy)cyclohexyl)-5-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyrazine-2-carboxamide (6.3 mg, 7.88 μmol, 4.50% yield). LCMS (high pH A): Rt=1.24 min, MH⁺=800.3

### Example AR199

### N-((1r,4r)-4-(3-Chloro-4-cyanophenoxy)cyclohexyl)-4-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)benzamide (EAR199)

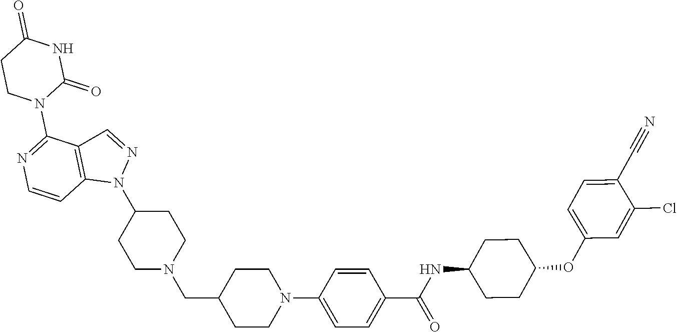 [SMILES: N#Cc1ccc(O[C@H]2CC[C@H](NC(=O)c3ccc(N4CCC(CN5CCC(n6ncc7c(N8CCC(=O)NC8=O)nccc76)CC5)CC4)cc3)CC2)cc1Cl]

N-((1r,4r)-4-(3-Chloro-4-cyanophenoxy)cyclohexyl)-4-(4-formylpiperidin-1-yl)benzamide (may be prepared as described in Description 266; 79 mg, 0.170 mmol) and 1-(1-(piperidin-4-yl)-1H-pyrazolo[4,3-c]pyridin-4-yl)dihydropyrimidine-2,4(1H,3H)-dione (may be prepared as described in Example ECB7; 50 mg, 0.159 mmol) were stirred in DCM (3 mL) for 10 min. Sodium triacetoxyborohydride (130 mg, 0.613 mmol) was added, and the reaction was stirred 2 h. The reaction mixture was partitioned between DCM (10 mL) and water (10 mL). The organic phase was separated. The aqueous phase was extracted with DCM (5 mL×1), and the combined organic phases were washed with water (15 mL) and brine (15 mL). The organic phase was dried and evaporated. The residue was purified by MDAP (high pH) to give N-((1r,4r)-4-(3-chloro-4-cyanophenoxy)cyclohexyl)-4-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)benzamide (51 mg, 0.064 mmol, 40.0% yield). LCMS (high pH A): Rt=1.24 min, MH⁺=764.2

### Example AR200

### N-((1r,4r)-4-(3-Chloro-4-cyanophenoxy)cyclohexyl)-2-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyrimidine-5-carboxamide hydrochloride (EAR200)

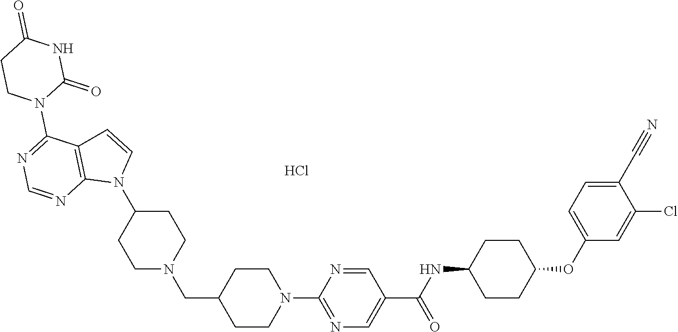 [SMILES: N#Cc1ccc(O[C@H]2CC[C@H](NC(=O)c3cnc(N4CCC(CN5CCC(n6ccc7c(N8CCC(=O)NC8=O)ncnc76)CC5)CC4)nc3)CC2)cc1Cl ; Cl]

N-((1r,4r)-4-(3-Chloro-4-cyanophenoxy)cyclohexyl)-2-(4-formylpiperidin-1-yl)pyrimidine-5-carboxamide (may be prepared as described in Description 103; 84 mg, 0.180 mmol) and 1-(7-(piperidin-4-yl)-7H-pyrrolo[2,3-d]pyrimidin-4-yl)dihydropyrimidine-2,4(1H,3H)-dione (may be prepared as described in Example ECB17; 50 mg, 0.159 mmol) were stirred in DCM (3 mL) for 10 min. Sodium triacetoxyborohydride (169 mg, 0.797 mmol) was added, and the reaction was stirred 21.5 h. The reaction mixture was partitioned between DCM (10 mL) and water (10 mL). The organic phase was separated. The aqueous phase was extracted with DCM (5 mL), and the combined organic phases were washed with water (15 mL) and brine (15 mL). The organic phase was dried and evaporated. The residue was purified by MDAP (high pH) to give material that was dissolved in DCM, treated with 2M HCl and concentrated to give N-((1r,4r)-4-(3-chloro-4-cyanophenoxy)cyclohexyl)-2-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyrimidine-5-carboxamide hydrochloride (7.4 mg, 9.22 μmol, 5.80% yield). LCMS (high pH A): Rt=1.18 min, MH⁺=766.2

### Example AR201

### N-((1r,4r)-4-((3-Chloro-4-cyanophenyl)(methyl)amino)cyclohexyl)-6-(4-((4-(4-(2,4-dioxotetrahydropyrimidin-1(2H)-yl)-6-fluoro-1H-indol-1-yl)piperidin-1-yl)methyl)piperidin-1-yl)pyridazine-3-carboxamide hydrochloride (EAR201)

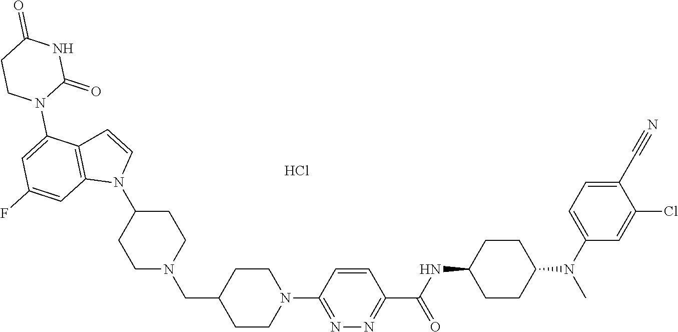 [SMILES: CN(c1ccc(C#N)c(Cl)c1)[C@H]1CC[C@H](NC(=O)c2ccc(N3CCC(CN4CCC(n5ccc6c(N7CCC(=O)NC7=O)cc(F)cc65)CC4)CC3)nn2)CC1 ; Cl]

To N-((1r,4r)-4-((3-chloro-4-cyanophenyl)(methyl)amino)cyclohexyl)-6-(4-formylpiperidin-1-yl)pyridazine-3-carboxamide (may be prepared as described in Description 317; 100 mg, 0.208 mmol) in DCM (2 ml) was added 1-(6-fluoro-1-(piperidin-4-yl)-1H-indol-4-yl)dihydropyrimidine-2,4(1H,3H)-dione (may be prepared as described in Example ECB44; 68.2 mg, 0.206 mmol). After 30 minutes, sodium triacetoxyhydroborate (88 mg, 0.415 mmol) was added. After 1.5 h, the mixture was diluted with DCM (8 mL) and water (10 mL), and the layers were separated. The aqueous layer was extracted with DCM (2×10 mL), and the combined organic layers were passed through a hydrophobic frit, concentrated and purified by MDAP (high pH) to give material that was dissolved in DCM (3 mL), cooled in an ice-water bath and treated with HCl (4 M in 1,4-dioxane, 23.6 μL, 1.5 eq). After 40 minutes the reaction was concentrated to give N-((1r,4r)-4-((3-chloro-4-cyanophenyl)(methyl)amino)cyclohexyl)-6-(4-((4-(4-(2,4-dioxotetrahydropyrimidin-1(2H)-yl)-6-fluoro-1H-indol-1-yl)piperidin-1-yl)methyl)piperidin-1-yl)pyridazine-3-carboxamide hydrochloride (33.4 mg, 0.040 mmol, 19.45% yield). LCMS (high pH A): Rt=1.32 min, MH⁺=795.1

### Example AR202

### N-((1r,4r)-4-(3-Chloro-4-cyanophenoxy)cyclohexyl)-5-(4-((4-(4-(2,4-dioxotetrahydropyrimidin-1(2H)-yl)-6-fluoro-1H-indol-1-yl)piperidin-1-yl)methyl)piperidin-1-yl)pyrazine-2-carboxamide hydrochloride (EAR202)

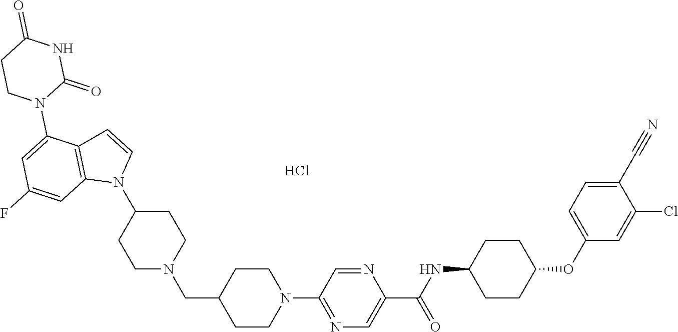 [SMILES: N#Cc1ccc(O[C@H]2CC[C@H](NC(=O)c3cnc(N4CCC(CN5CCC(n6ccc7c(N8CCC(=O)NC8=O)cc(F)cc76)CC5)CC4)cn3)CC2)cc1Cl ; Cl]

To N-((1r,4r)-4-(3-chloro-4-cyanophenoxy)cyclohexyl)-5-(4-formylpiperidin-1-yl)pyrazine-2-carboxamide (may be prepared as described in Description 99; 102 mg, 0.218 mmol) in DCM (2 ml) was added 1-(6-fluoro-1-(piperidin-4-yl)-1H-indol-4-yl)dihydropyrimidine-2,4(1H,3H)-dione (may be prepared as described in Example ECB44; 72 mg, 0.218 mmol). After 30 minutes, sodium triacetoxyhydroborate (92 mg, 0.436 mmol) was added. After 2 h, the mixture was diluted with DCM (8 mL) and water (10 mL), and the layers were separated. The aqueous layer was extracted with DCM (10 mL), and the combined organic layers were passed through a hydrophobic frit, concentrated and purified by MDAP (formic) to give material that was dissolved in DCM (2.5 mL), cooled in an ice-water bath and treated with HCl (4 M in 1,4-dioxane, 40.5 μL, 1.5 eq). After 45 minutes the reaction was concentrated to give N-((1r,4r)-4-(3-chloro-4-cyanophenoxy)cyclohexyl)-5-(4-((4-(4-(2,4-dioxotetrahydropyrimidin-1(2H)-yl)-6-fluoro-1H-indol-1-yl)piperidin-1-yl)methyl)piperidin-1-yl)pyrazine-2-carboxamide hydrochloride (46.5 mg, 0.057 mmol, 26.1% yield). LCMS (high pH A): Rt=1.32 min, MH⁺=782.2

### Example AR203

### N-((1r,4r)-4-(3-Chloro-4-cyanophenoxy)cyclohexyl)-5-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)pyrazine-2-carboxamide hydrochloride (EAR203)

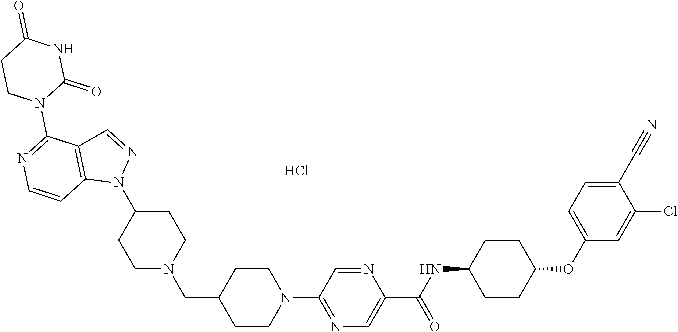 [SMILES: N#Cc1ccc(O[C@H]2CC[C@H](NC(=O)c3cnc(N4CCC(CN5CCC(n6ncc7c(N8CCC(=O)NC8=O)nccc76)CC5)CC4)cn3)CC2)cc1Cl ; Cl]

N-((1r,4r)-4-(3-Chloro-4-cyanophenoxy)cyclohexyl)-5-(4-formylpiperidin-1-yl)pyrazine-2-carboxamide (may be prepared as described in Description 99; 77 mg, 0.165 mmol) and 1-(1-(piperidin-4-yl)-1H-pyrazolo[4,3-c]pyridin-4-yl)dihydropyrimidine-2,4(1H,3H)-dione (may be prepared as described in Example ECB7; 50 mg, 0.159 mmol) was stirred in DCM (3 mL) for 10 min. Sodium triacetoxyborohydride (158 mg, 0.745 mmol) was added, and the reaction was stirred for 4 h. The mixture was partitioned between DCM (10 mL) and water (10 mL), and the organic phase was separated. The aqueous phase was extracted with DCM (5 ml×1). The combined organic phases were washed with water (15 mL) and brine (15 mL), dried, evaporated and purified by MDAP (high pH) to yield material that was dissolved in chloroform-D and methanol. To this suspension, 2M hydrogen chloride was added, then the solvent was removed to give N-((1r,4r)-4-(3-chloro-4-cyanophenoxy)cyclohexyl)-5-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)pyrazine-2-carboxamide hydrochloride (7.2 mg, 8.97 μmol, 5.64% yield). LCMS (high pH A): Rt=1.24 min, MH⁺=766.2, 768.2

### Example AR204

### N-((1r,4r)-4-(4-Cyano-3-(trifluoromethyl)phenoxy)cyclohexyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyridazine-3-carboxamide (EAR204)

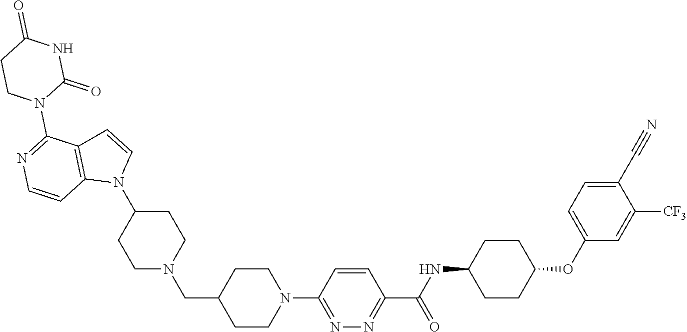 [SMILES: N#Cc1ccc(O[C@H]2CC[C@H](NC(=O)c3ccc(N4CCC(CN5CCC(n6ccc7c(N8CCC(=O)NC8=O)nccc76)CC5)CC4)nn3)CC2)cc1C(F)(F)F]

N-((1r,4r)-4-(4-Cyano-3-(trifluoromethyl)phenoxy)cyclohexyl)-6-(4-formylpiperidin-1-yl)pyridazine-3-carboxamide (may be prepared as described in Description 97; 52 mg, 0.112 mmol) and 1-(1-(piperidin-4-yl)-1H-pyrrolo[3,2-c]pyridin-4-yl)dihydropyrimidine-2,4(1H,3H)-dione (may be prepared as described in Example ECB25; 35 mg, 0.112 mmol) were stirred in DCM (3 mL) for 2 h. Sodium triacetoxyborohydride (71 mg, 0.335 mmol) was added, and the reaction was stirred 17 h. Saturated sodium bicarbonate solution (5 mL) was added, and the mixture was stirred for 10 minutes. The organic phase was separated. The aqueous phase was extracted with dichloromethane (5 ml), and the combined organics were dried, evaporated and purified by MDAP (high pH) to give N-((1r,4r)-4-(4-cyano-3-(trifluoromethyl)phenoxy)cyclohexyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyridazine-3-carboxamide (24 mg, 0.030 mmol, 26.9% yield). LCMS (high pH A): Rt=1.23 min, MH⁺=799.2

### Example AR205

### N-((1r,4r)-4-(3-Chloro-4-cyanophenoxy)cyclohexyl)-2-(4-((4-(4-(2,4-dioxotetrahydropyrimidin-1(2H)-yl)-6-fluoro-1H-indazol-1-yl)piperidin-1-yl)methyl)piperidin-1-yl)pyrimidine-5-carboxamide (AR205)

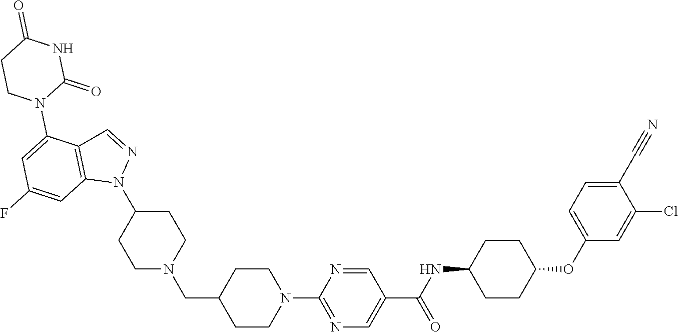 [SMILES: N#Cc1ccc(O[C@H]2CC[C@H](NC(=O)c3cnc(N4CCC(CN5CCC(n6ncc7c(N8CCC(=O)NC8=O)cc(F)cc76)CC5)CC4)nc3)CC2)cc1Cl]

1-(6-Fluoro-1-(piperidin-4-yl)-1H-indazol-4-yl)dihydropyrimidine-2,4(1H,3H)-dione (may be prepared as described in Example ECB10; 40 mg, 0.121 mmol) and N-((1r,4r)-4-(3-chloro-4-cyanophenoxy)cyclohexyl)-2-(4-formylpiperidin-1-yl)pyrimidine-5-carboxamide (may be prepared as described in Description 103; 60 mg, 0.128 mmol) in dichloromethane (3 mL) were stirred at room temperature for 2 h. Sodium triacetoxyborohydride (77 mg, 0.362 mmol) was added, and the reaction mixture was stirred temperature for 17 h. Saturated sodium bicarbonate solution (5 mL) was added and the mixture was stirred for 10 minutes. The organic phase was separated, and the aqueous phase was extracted with dichloromethane (5 mL). The combined organics were dried and evaporated. The residue was suspended in hot methanol (3 mL) and filtered. The precipitate was washed with methanol and dried to give N-((1r,4r)-4-(3-chloro-4-cyanophenoxy)cyclohexyl)-2-(4-((4-(4-(2,4-dioxotetrahydropyrimidin-1(2H)-yl)-6-fluoro-1H-indazol-1-yl)piperidin-1-yl)methyl)piperidin-1-yl)pyrimidine-5-carboxamide (67 mg, 0.086 mmol, 70.9% yield). LCMS (high pH A): Rt=1.25 min, MH⁺=783.2

### Example AR206

### N-((1r,4r)-4-(3-Chloro-4-cyanophenoxy)cyclohexyl)-4-(4-((4-(4-(2,4-dioxotetrahydropyrimidin-1(2H)-yl)-1H-indol-1-yl)piperidin-1-yl)methyl)piperidin-1-yl)-3-fluorobenzamide (EAR206)

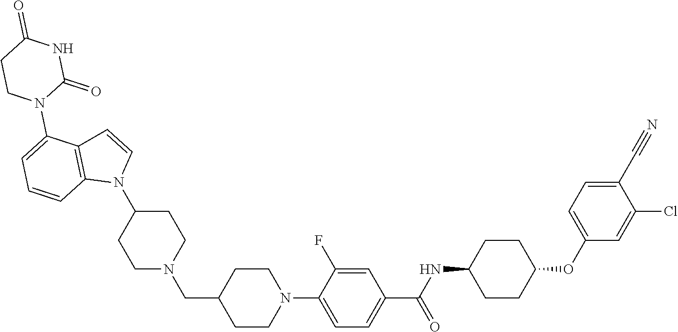 [SMILES: N#Cc1ccc(O[C@H]2CC[C@H](NC(=O)c3ccc(N4CCC(CN5CCC(n6ccc7c(N8CCC(=O)NC8=O)cccc76)CC5)CC4)c(F)c3)CC2)cc1Cl]

N-((1r,4r)-4-(3-chloro-4-cyanophenoxy)cyclohexyl)-3-fluoro-4-(4-formylpiperidin-1-yl)benzamide (may be prepared as described in Description 321; 55 mg, 0.114 mmol) and 1-(1-(piperidin-4-yl)-1H-indol-4-yl)dihydropyrimidine-2,4(1H,3H)-dione (may be prepared as described in Example ECB1; 42 mg, 0.134 mmol) were stirred in DCM (3 mL). After 1 h 40 min, sodium triacetoxyborohydride (72.3 mg, 0.341 mmol) was added. After 1 h, the reaction was partitioned between DCM (10 mL) and saturated hydrogen carbonate (10 mL), and the organic phase was separated. The aqueous phase was extracted with DCM (5 mL×2). The combined organic phases were washed with water (5 ml) and brine (5 mL), dried, evaporated and purified by MDAP (high pH) to give N-((1r,4r)-4-(3-chloro-4-cyanophenoxy)cyclohexyl)-4-(4-((4-(4-(2,4-dioxotetrahydropyrimidin-1(2H)-yl)-1H-indol-1-yl)piperidin-1-yl)methyl)piperidin-1-yl)-3-fluorobenzamide (15.6 mg, 0.020 mmol, 17.59% yield). LCMS (high pH A): Rt=1.36 min, MH⁺=780.2

### Examples AR207 and AR208

### N-((1r,4r)-4-(3-Chloro-4-cyanophenoxy)cyclohexyl)-6-(4-(((1s,3s)-3-(4-(2,4-dioxotetrahydropyrimidin-1(2H)-yl)-1H-indol-1-yl)cyclobutyl)methyl)piperazin-1-yl)pyridazine-3-carboxamide hydrochloride (EAR207) and

### N-((1r,4r)-4-(3-Chloro-4-cyanophenoxy)cyclohexyl)-6-(4-(((1r,3r)-3-(4-(2,4-dioxotetrahydropyrimidin-1(2H)-yl)-1H-indol-1-yl)cyclobutyl)methyl)piperazin-1-yl)pyridazine-3-carboxamide hydrochloride (EAR208)

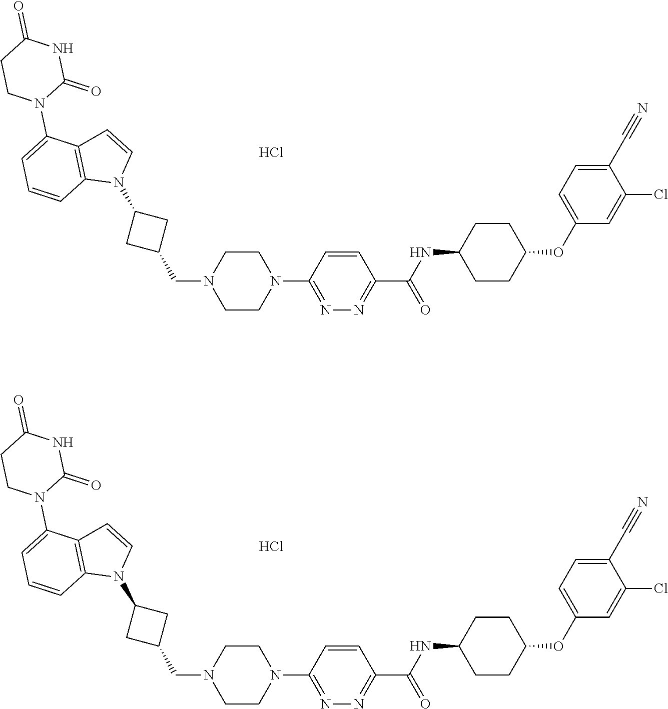 [SMILES: N#Cc1ccc(O[C@H]2CC[C@H](NC(=O)c3ccc(N4CCN(C[C@H]5C[C@@H](n6ccc7c(N8CCC(=O)NC8=O)cccc76)C5)CC4)nn3)CC2)cc1Cl ; Cl ; N#Cc1ccc(O[C@H]2CC[C@H](NC(=O)c3ccc(N4CCN(C[C@H]5C[C@H](n6ccc7c(N8CCC(=O)NC8=O)cccc76)C5)CC4)nn3)CC2)cc1Cl ; Cl]

To 3-(4-(2,4-dioxo-3-((2-(trimethylsilyl)ethoxy)methyl)tetrahydropyrimidin-1(2H)-yl)-1H-indol-1-yl)cyclobutane-1-carbaldehyde (may be prepared as described in Description 325; 81 mg, 0.183 mmol) and N-((1r,4r)-4-(3-chloro-4-cyanophenoxy)cyclohexyl)-6-(piperazin-1-yl)pyridazine-3-carboxamide, trifluoroacetic acid salt (may be prepared as described in Description 121; 102 mg, 0.183 mmol) in DCM (5 mL) was added acetic acid (0.032 mL, 0.550 mmol) was added. After 15 minutes sodium triacetoxyborohydride (97 mg, 0.459 mmol) was added. After 6 h, trifluoroacetic acid (0.283 mL, 3.67 mmol) was added, followed 2 h later by additional trifluoroacetic acid (0.283 mL, 3.67 mmol). The reaction was left to stand overnight, followed by cooling in an ice-water bath and the slow addition of ammonia (7 M in methanol) (2.98 mL, 20.87 mmol). After 10 mins the reaction was warmed to rt and stirred for 2 h. The mixture was concentrated, taken up in EtOAc (10 mL) and diluted with sat. aq. sodium bicarbonate (10 mL). The layers were separated, and the aqueous layer was extracted with EtOAc (10 mL). The combined organic layers were filtered through a hydrophobic frit, concentrated and purified through a Zorbax SB-Phenyl (150×30 mm, 5 μm) column, eluting with 40-100% MeCN (0.1% trifluoroacetic acid) in water (0.1% TFA) to yield material that was further purified through a 4.6 mm id×25 cm Chiral ART Cellulose SJ (5 μm) column, eluting with 1:1 MeCN (0.2% isopropylamine):water (0.2% isopropylamine) to yield the first-(46 mg, 0.062 mmol, 34% yield) and second-eluting (20 mg, 0.027 mmol, 15% yield) isomers. The first-eluting isomer was suspended in 1:1 MeCN:H₂O (2 mL) and treated with HCl (4 M in 1,4-dioxane, 28 μL, 1.8 eq). After 30 min, the reaction was concentrated to give N-((1r,4r)-4-(3-chloro-4-cyanophenoxy)cyclohexyl)-6-(4-(((1s,3s)-3-(4-(2,4-dioxotetrahydropyrimidin-1(2H)-yl)-1H-indol-1-yl)cyclobutyl)methyl)piperazin-1-yl)pyridazine-3-carboxamide hydrochloride (37 mg, 0.048 mmol). LCMS (high pH A): Rt=1.18 min, MH⁺=736.3.

The second-eluting isomer was suspended in 1:1 MeCN:H₂O (2 mL) and treated with HCl (4 M in 1,4-dioxane, 15 μL, 2.3 eq). After 30 min, the reaction was concentrated to give N-((1r,4r)-4-(3-chloro-4-cyanophenoxy)cyclohexyl)-6-(4-(((1r,3r)-3-(4-(2,4-dioxotetrahydropyrimidin-1(2H)-yl)-1H-indol-1-yl)cyclobutyl)methyl)piperazin-1-yl)pyridazine-3-carboxamide hydrochloride (15 mg, 0.019 mmol). LCMS (high pH A): Rt=1.20 min, MH⁺=736.2

### Example AR209

### N-((1r,4r)-4-(4-cyano-3-(trifluoromethyl)phenoxy)cyclohexyl)-6-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)pyridazine-3-carboxamide (EAR209)

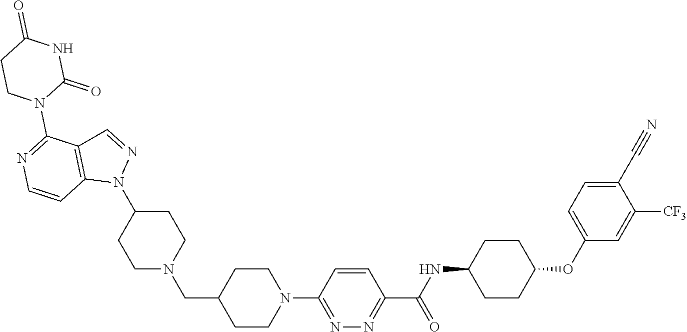 [SMILES: N#Cc1ccc(O[C@H]2CC[C@H](NC(=O)c3ccc(N4CCC(CN5CCC(n6ncc7c(N8CCC(=O)NC8=O)nccc76)CC5)CC4)nn3)CC2)cc1C(F)(F)F]

N-((1r,4r)-4-(4-Cyano-3-(trifluoromethyl)phenoxy)cyclohexyl)-6-(4-formylpiperidin-1-yl)pyridazine-3-carboxamide (may be prepared as described in Description 97; 98 mg, 0.195 mmol) and 1-(1-(piperidin-4-yl)-1H-pyrazolo[4,3-c]pyridin-4-yl)dihydropyrimidine-2,4(1H,3H)-dione (may be prepared as described in Example ECB7; 35 mg, 0.111 mmol) in DCM (5 mL) were stirred for 1 h. Sodium triacetoxyborohydride (72 mg, 0.340 mmol) was added, and after 1.5 h the mixture was partitioned between DCM (10 mL) and saturated hydrogen carbonate (10 mL). The organic phase was separated, and the aqueous phase was extracted with DCM (5 ml×2). The combined organic phases were washed with water (5 mL) and brine (5 mL), dried, concentrated and purified by MDAP (high pH) to give N-((1r,4r)-4-(4-cyano-3-(trifluoromethyl)phenoxy)cyclohexyl)-6-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)pyridazine-3-carboxamide (41 mg, 0.051 mmol, 46.0% yield). LCMS (high pH A): Rt=1.20 min, MH⁺=800.3

### Example AR210

### N-((1r,4r)-4-(4-Cyano-3-(trifluoromethyl)phenoxy)cyclohexyl)-6-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)pyridazine-3-carboxamide (EAR210)

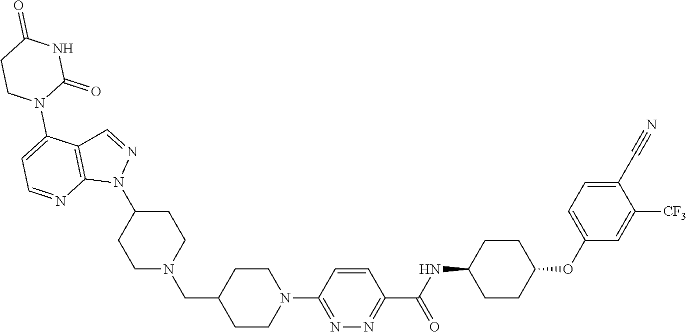 [SMILES: N#Cc1ccc(O[C@H]2CC[C@H](NC(=O)c3ccc(N4CCC(CN5CCC(n6ncc7c(N8CCC(=O)NC8=O)ccnc76)CC5)CC4)nn3)CC2)cc1C(F)(F)F]

N-((1r,4r)-4-(4-Cyano-3-(trifluoromethyl)phenoxy)cyclohexyl)-6-(4-formylpiperidin-1-yl)pyridazine-3-carboxamide (may be prepared as described in Description 97; 54 mg, 0.108 mmol) and 1-(1-(piperidin-4-yl)-1H-pyrazolo[3,4-b]pyridin-4-yl)dihydropyrimidine-2,4(1H,3H)-dione (may be prepared as described in Example ECB19; 34 mg, 0.108 mmol) in DCM (3 mL) were stirred for 1 h. Sodium triacetoxyborohydride (84 mg, 0.396 mmol) was added, and after 16.5 h the mixture was partitioned between DCM (10 mL) and saturated hydrogen carbonate (10 mL). The organic phase was separated, and the aqueous phase was extracted with DCM (5 ml×2). The combined organic phases were washed with water (5 mL) and brine (5 mL), dried, concentrated and purified by MDAP (high pH) to give N-((1r,4r)-4-(4-cyano-3-(trifluoromethyl)phenoxy)cyclohexyl)-6-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)pyridazine-3-carboxamide (24 mg, 0.030 mmol, 27.9% yield). LCMS (high pH A): Rt=1.20 min, MH⁺=800.3

### Example AR211

### N-((1r,4r)-4-(3-chloro-4-cyanophenoxy)cyclohexyl)-2-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyrimidine-5-carboxamide hydrochloride (EAR211)

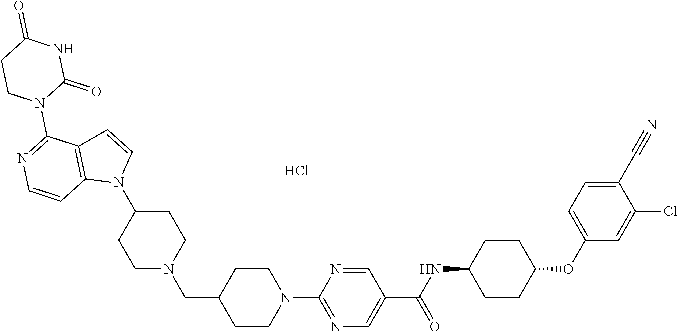 [SMILES: N#Cc1ccc(O[C@H]2CC[C@H](NC(=O)c3cnc(N4CCC(CN5CCC(n6ccc7c(N8CCC(=O)NC8=O)nccc76)CC5)CC4)nc3)CC2)cc1Cl ; Cl]

N-((1r,4r)-4-(3-Chloro-4-cyanophenoxy)cyclohexyl)-2-(4-formylpiperidin-1-yl)pyrimidine-5-carboxamide (may be prepared as described in Description 103; 100 mg, 0.214 mmol) and 1-(1-(piperidin-4-yl)-1H-pyrrolo[3,2-c]pyridin-4-yl)dihydropyrimidine-2,4(1H,3H)-dione (may be prepared as described in Example ECB25; 67 mg, 0.214 mmol) in DCM (2 mL) were stirred for 35 min. Sodium triacetoxyborohydride (97 mg, 0.458 mmol) was added, and after 1.5 h the mixture was partitioned between DCM (8 mL), brine (1 mL) and water (10 mL). The organic phase was separated, and the aqueous phase was extracted with DCM (10 mL×2). The combined organic phases were passed through a hydrophobic frit, dried, concentrated and purified by MDAP (formic) to give was material that was diluted with MeCN:water (1:1, 2 ml), cooled in an ice-water bath, and treated with HCl (4 M in 1,4-dioxane, 27.8 μL, 1.5 eq). After 45 minutes the reaction was concentrated to give N-((1r,4r)-4-(3-chloro-4-cyanophenoxy)cyclohexyl)-2-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyrimidine-5-carboxamide hydrochloride (65.3 mg, 0.081 mmol, 38.1% yield). LCMS (formic A): Rt=0.70 min, MH⁺=765.3, 767.2

### Example AR212

### N-((1r,4r)-4-(4-Cyano-3-(trifluoromethyl)phenoxy)cyclohexyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyridazine-3-carboxamide (EAR212)

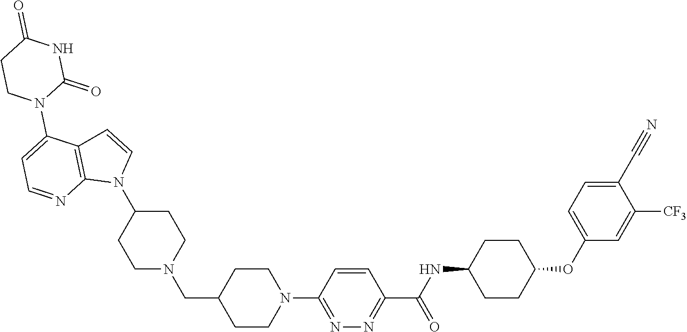 [SMILES: N#Cc1ccc(O[C@H]2CC[C@H](NC(=O)c3ccc(N4CCC(CN5CCC(n6ccc7c(N8CCC(=O)NC8=O)ccnc76)CC5)CC4)nn3)CC2)cc1C(F)(F)F]

N-((1r,4r)-4-(4-Cyano-3-(trifluoromethyl)phenoxy)cyclohexyl)-6-(4-formylpiperidin-1-yl)pyridazine-3-carboxamide (may be prepared as described in Description 97; 54 mg, 0.108 mmol) and 1-(1-(piperidin-4-yl)-1H-pyrrolo[2,3-b]pyridin-4-yl)dihydropyrimidine-2,4(1H,3H)-dione (may be prepared as described in Example ECB26; 34 mg, 0.109 mmol) were stirred in DCM (3 mL). After 2 h h, sodium triacetoxyborohydride (84 mg, 0.396 mmol) was added. After 1 h 50 minutes the mixture was partitioned between DCM (10 mL) and saturated sodium hydrogen carbonate (10 mL). The organic phase was separated, and the aqueous phase was extracted with DCM (5 mL×2). The combined organic phases were washed with water (5 mL) and brine (5 mL), dried, concentrated and purified by MDAP (high pH) to give N-((1r,4r)-4-(4-cyano-3-(trifluoromethyl)phenoxy)cyclohexyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyridazine-3-carboxamide (14.5 mg, 0.018 mmol, 16.86% yield). LCMS (high pH A): Rt=1.22 min, MH⁺=799.2

### Example AR213

### N-((1r,4r)-4-(3-Chloro-4-cyanophenoxy)cyclohexyl)-6-(4-((4-(4-(2,4-dioxotetrahydropyrimidin-1(2H)-yl)-1H-indol-1-yl)piperidin-1-yl)methyl)-4-methylpiperidin-1-yl)pyridazine-3-carboxamide (EAR213)

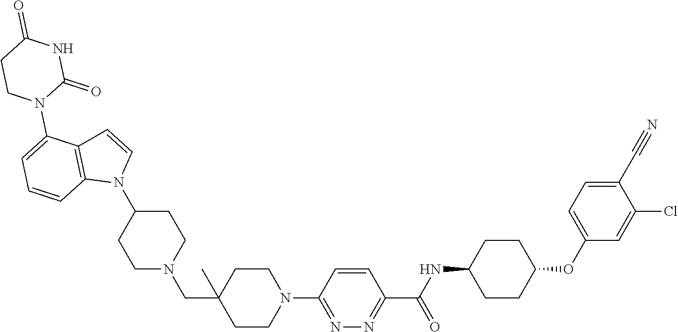 [SMILES: CC1(CN2CCC(n3ccc4c(N5CCC(=O)NC5=O)cccc43)CC2)CCN(c2ccc(C(=O)N[C@H]3CC[C@H](Oc4ccc(C#N)c(Cl)c4)CC3)nn2)CC1]

N-((1r,4r)-4-(3-Chloro-4-cyanophenoxy)cyclohexyl)-6-(4-formyl-4-methylpiperidin-1-yl)pyridazine-3-carboxamide (may be prepared as described in Description 327; 45 mg, 0.093 mmol) and 1-(1-(piperidin-4-yl)-1H-indol-4-yl)dihydropyrimidine-2,4(1H,3H)-dione (may be prepared as described in Example ECB1; 30 mg, 0.096 mmol) were stirred in DCM (3 mL). After 2 hours and 35 minutes 2-methylpyridine borane complex (Picoline Borane) (21 mg, 0.196 mmol) was added. After 1.5 h, ethanol (3 mL) was added to the reaction, and the solvent DCM was evaporated. Additional 2-methylpyridine borane complex (Picoline Borane) (23 mg, 0.215 mmol) was added, and the reaction was heated to 60° C. After 17 h, another portion of 2-methylpyridine borane complex (Picoline Borane) (46 mg, 0.430 mmol) was added. After 2 h, the reaction was concentrated and purified by MDAP (high pH) to give N-((1r,4r)-4-(3-chloro-4-cyanophenoxy)cyclohexyl)-6-(4-((4-(4-(2,4-dioxotetrahydropyrimidin-1(2H)-yl)-1H-indol-1-yl)piperidin-1-yl)methyl)-4-methylpiperidin-1-yl)pyridazine-3-carboxamide (5.2 mg, 6.68 μmol, 7.16% yield). LCMS (high pH A): Rt=1.38 min, MH⁺=778.3

### Example AR214

### N-((1r,4r)-4-((3-Chloro-4-cyanophenyl)(methyl)amino)cyclohexyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide hydrochloride (EAR214)

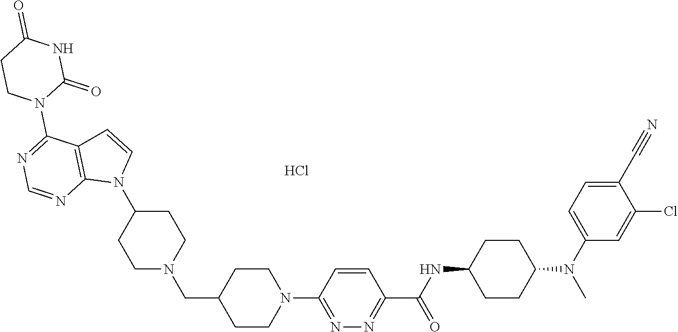 [SMILES: CN(c1ccc(C#N)c(Cl)c1)[C@H]1CC[C@H](NC(=O)c2ccc(N3CCC(CN4CCC(n5ccc6c(N7CCC(=O)NC7=O)ncnc65)CC4)CC3)nn2)CC1 ; Cl]

1-(7-(Piperidin-4-yl)-7H-pyrrolo[2,3-d]pyrimidin-4-yl)dihydropyrimidine-2,4(1H,3H)-dione (may be prepared as described in Example ECB17; 66.1 mg, 0.210 mmol) and N-((1r,4r)-4-((3-chloro-4-cyanophenyl)(methyl)amino)cyclohexyl)-6-(4-formylpiperidin-1-yl)pyridazine-3-carboxamide (may be prepared as described in Description 317; 100 mg, 0.208 mmol) were stirred in DCM (2 ml). After 15 minutes sodium triacetoxyhydroborate (88 mg, 0.416 mmol) was added. After 1.5 h, the mixture was diluted with DCM (10 mL) and water (12 mL), and the layers were separated. The aqueous layer was extracted with DCM (2×12 mL), and the combined organic layers were passed through a hydrophobic frit, concentrated and purified by MDAP (formic) to give material that was dissolved in DCM (0.9 ml) and treated with HCl (4 M in 1,4-dioxane, 19.2 μL, 1.5 eq). After 35 minutes the reaction was concentrated to give N-((1r,4r)-4-((3-chloro-4-cyanophenyl)(methyl)amino)cyclohexyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide hydrochloride (38.2 mg, 0.047 mmol, 22.52% yield). LCMS (formic A): Rt=0.77 min, MH⁺=779.3

### Example AR215

### N-((1r,4r)-4-(3-Chloro-4-cyanophenoxy)cyclohexyl)-6-(4-(((1r,4r)-4-(4-(2,4-dioxotetrahydropyrimidin-1(2H)-yl)-1H-indol-1-yl)cyclohexyl)methyl)piperazin-1-yl)pyridazine-3-carboxamide hydrochloride (AR215)

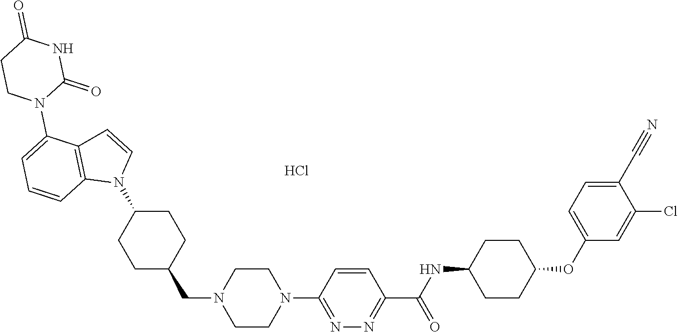 [SMILES: N#Cc1ccc(O[C@H]2CC[C@H](NC(=O)c3ccc(N4CCN(C[C@H]5CC[C@H](n6ccc7c(N8CCC(=O)NC8=O)cccc76)CC5)CC4)nn3)CC2)cc1Cl ; Cl]

To ((1r,4r)-4-(4-(2,4-dioxo-3-((2-(trimethylsilyl)ethoxy)methyl)tetrahydropyrimidin-1(2H)-yl)-1H-indol-1-yl)cyclohexyl)methyl 4-methylbenzenesulfonate (may be prepared as described in Description 328; 269 mg, 0.430 mmol) and sodium bicarbonate (108 mg, 1.289 mmol) in DMF (3 mL) was added N-((1r,4r)-4-(3-chloro-4-cyanophenoxy)cyclohexyl)-6-(piperazin-1-yl)pyridazine-3-carboxamide trifluoroacetic acid salt (may be prepared as described Description 121; 190 mg, 0.430 mmol). The reaction was heated to 80° C. for 24 h then to 90° C. for 4 h. A further portion of sodium bicarbonate (108 mg, 1.289 mmol) was added, and the vial was sealed and stirred at 80° C. for a further 65 h. The mixture was cooled to room temperature and diluted with DCM (10 mL), sat. aq. sodium bicarbonate solution (10 mL) and water (5 mL), and the layers were separated. The aqueous layer was extracted with DCM (6 mL), and the combined organic layers were filtered through a hydrophobic frit, concentrated, dissolved in DCM (3 mL), and treated with TFA (0.993 mL, 12.89 mmol). After 2 h, the reaction was concentrated and redissolved in MeOH (3 mL) and DCM (3 mL), followed by the addition of ammonia (7 N in methanol) (1.842 mL, 12.89 mmol). After 1 h, the reaction was further diluted with DCM (8 mL), and saturated aqueous sodium hydrogen carbamate solution (8 mL) and water (2 mL). The layers were separated, and the aqueous layer was extracted with DCM (6 mL). The combined organic layers were filtered through a hydrophobic frit, concentrated, dissolved in DMSO (1 mL) and purified using an XSelect C18 OBD column, eluting with 50-100% MeCN in 10 mM ammonium bicarbonate solution to give material that was dissolved in DCM (1 mL) and treated with HCl (4 M in 1,4-dioxane, 15 μL, 1.5 eq). After 1 h the reaction was concentrated to give N-((1r,4r)-4-(3-chloro-4-cyanophenoxy)cyclohexyl)-6-(4-(((1r,4r)-4-(4-(2,4-dioxotetrahydropyrimidin-1(2H)-yl)-1H-indol-1-yl)cyclohexyl)methyl)piperazin-1-yl)pyridazine-3-carboxamide, Hydrochloride (31.7 mg, 0.040 mmol, 9.21% yield). LCMS (high pH A): Rt=1.30 min, MH⁺=764.3

### Example AR216

### N-((1r,4r)-4-(3-chloro-4-cyanophenoxy)cyclohexyl)-2-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyrimidine-5-carboxamide (AR216)

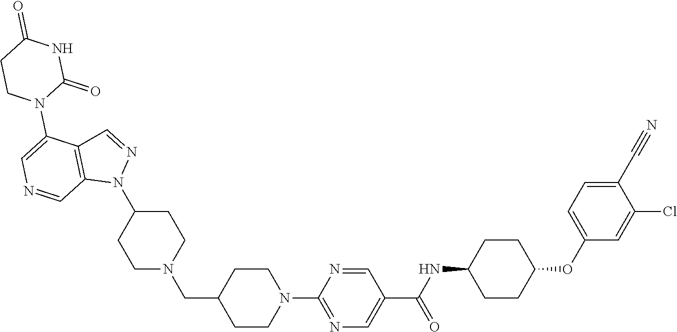 [SMILES: N#Cc1ccc(O[C@H]2CC[C@H](NC(=O)c3cnc(N4CCC(CN5CCC(n6ncc7c(N8CCC(=O)NC8=O)cncc76)CC5)CC4)nc3)CC2)cc1Cl]

N-((1r,4r)-4-(3-Chloro-4-cyanophenoxy)cyclohexyl)-2-(4-formylpiperidin-1-yl)pyrimidine-5-carboxamide (may be prepared as described in Description 103; 50 mg, 0.107 mmol) and 1-(1-(piperidin-4-yl)-1H-pyrazolo[3,4-c]pyridin-4-yl)dihydropyrimidine-2,4(1H,3H)-dione (may be prepared as described in Example ECB8; 41 mg, 0.130 mmol) in dichloromethane (3 mL) were stirred for 2 hours. Sodium triacetoxyborohydride (68 mg, 0.3221 mmol) was added, and the reaction was stirred for 2 hours, and diluted with dichloromethane (5 mL) and saturated sodium bicarbonate solution (5 mL). After 5 minutes, the organic phase was separated. The aqueous phase was extracted with dichloromethane (2×5 mL), and the combined organics were dried, concentrated suspended in methanol (3 mL) and heated to 40° C. After 30 minutes the solid was collected by filtration to give N-((1r,4r)-4-(3-chloro-4-cyanophenoxy)cyclohexyl)-2-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyrimidine-5-carboxamide (61 mg, 0.080 mmol, 74.5% yield). LCMS (high pH A): Rt=1.12 min, MH⁺=766.3

### Example AR217

### N-((1r,4r)-4-(3-chloro-4-cyanophenoxy)cyclohexyl)-5-(4-((4-(4-(2,4-dioxotetrahydropyrimidin-1(2H)-yl)-6-fluoro-1H-indazol-1-yl)piperidin-1-yl)methyl)piperidin-1-yl)pyrazine-2-carboxamide hydrochloride (EAR217)

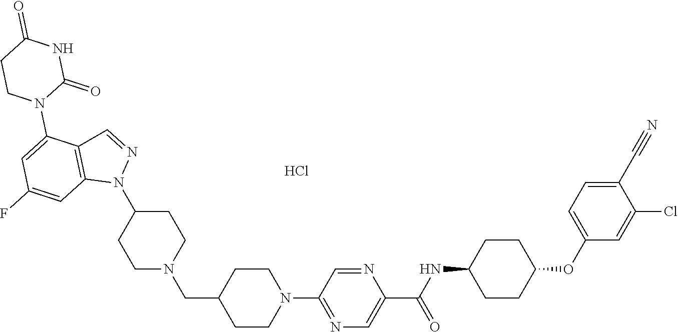 [SMILES: N#Cc1ccc(O[C@H]2CC[C@H](NC(=O)c3cnc(N4CCC(CN5CCC(n6ncc7c(N8CCC(=O)NC8=O)cc(F)cc76)CC5)CC4)cn3)CC2)cc1Cl ; Cl]

N-((1r,4r)-4-(3-Chloro-4-cyanophenoxy)cyclohexyl)-5-(4-formylpiperidin-1-yl)pyrazine-2-carboxamide (may be prepared as described in Description 99; 93 mg, 0.199 mmol) and 1-(6-fluoro-1-(piperidin-4-yl)-1H-indazol-4-yl)dihydropyrimidine-2,4(1H,3H)-dione (may be prepared as described in Example ECB10; 60 mg, 0.181 mmol) were suspended in DCM (2.5 mL). After 10 min, sodium triacetoxyborohydride (115 mg, 0.543 mmol) was added. After 16 h the mixture was diluted with DCM (20 mL), NaHCO₃ (15 mL) and brine (5 mL) and shaken vigorously. The layers were separated, and the aqueous was extracted with DCM (10 mL). The organics were combined, dried by passing through a hydrophobic frit, concentrated and purified to give material that was taken up in 1,4-dioxane (500 μL) and treated with HCl (4 M in dioxane) (0.072 mL, 0.288 mmol). After 2 h the reaction was concentrated to give N-((1r,4r)-4-(3-chloro-4-cyanophenoxy)cyclohexyl)-5-(4-((4-(4-(2,4-dioxotetrahydropyrimidin-1(2H)-yl)-6-fluoro-1H-indazol-1-yl)piperidin-1-yl)methyl)piperidin-1-yl)pyrazine-2-carboxamide hydrochloride (67.8 mg, 0.083 mmol, 45.7% yield). LCMS (high pH A): Rt=1.31 min, MH⁺=781.5, 783.2

### Examples EAR218-EAR228

In eleven separate reactions, to the amines (0.155 mmol) listed in the table below were added 6-chloro-N-((1r,4r)-4-(3-chloro-4-cyanophenoxy)cyclohexyl)pyridazine-3-carboxamide (may be prepared as described in Description 90; 0.031 g, 0.08 mmol) and DIPEA (0.135 mL, 0.775 mmol) dissolved in DMSO (1.15 mL). The reactions were left to stir 60° C. for 20 h, then at 80° C. for 18 h. An additional 5 eq of DIPEA (70 μL) was added, and the reactions were heated to 80° C. for 34 h, then concentrated under a stream of nitrogen at 40° C. Reactions were purified by MDAP (high pH or formic, as indicated below) to give the following compounds:

| | | | Puri- |
|---|---|---|---|
| | | | fication |
| | | | and |
| | Product structure and name | | Analyt- |
| Example | Mass, yield | Amine | ical |
| EAR218 | 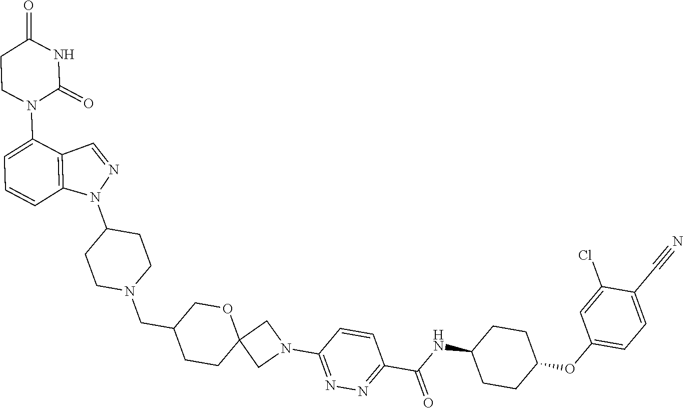 [SMILES: N#Cc1ccc(O[C@H]2CC[C@H](NC(=O)c3ccc(N4CC5(CCC(CN6CCC(n7ncc8c(N9CCC(=O)NC9=O)cccc87)CC6)CO5)C4)nn3)CC2)cc1Cl] | Example CBL13 | MDAP: formic LCMS (formic A): Rt = 1.17 min, MH⁺ = 807 |
| | N-((1r,4r)-4-(3-Chloro-4-cyanophenoxy)cyclohexyl)-6-(7-((4-(4- | | |
| | (2,4-dioxotetrahydropyrimidin-1(2H)-yl)-1H-indazol-1- | | |
| | yl)piperidin-1-yl)methyl)-5-oxa-2-azaspiro[3.5]nonan-2- | | |
| | yl)pyridazine-3-carboxamide | | |
| | 14.8 mg, 21.77% | | |
| EAR219 | 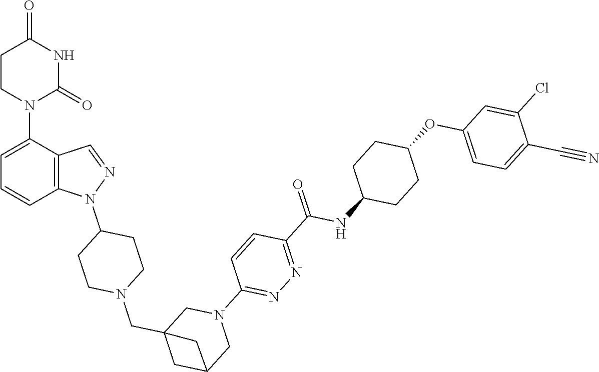 [SMILES: N#Cc1ccc(O[C@H]2CC[C@H](NC(=O)c3ccc(N4CC5CC(CN6CCC(n7ncc8c(N9CCC(=O)NC9=O)cccc87)CC6)(C5)C4)nn3)CC2)cc1Cl] | Example CBL14 | MDAP: high pH LCMS (high pH A): Rt = 1.26 min, MH⁺ = 777 |
| | N-((1r,4r)-4-(3-chloro-4-cyanophenoxy)cyclohexyl)-6-(1-((4-(4- | | |
| | (2,4-dioxotetrahydropyrimidin-1(2H)-yl)-1H-indazol-1- | | |
| | yl)piperidin-1-yl)methyl)-3-azabicyclo[3.1.1]heptan-3- | | |
| | yl)pyridazine-3-carboxamide | | |
| | 15.5 mg, 23.68% | | |
| EAR220 | 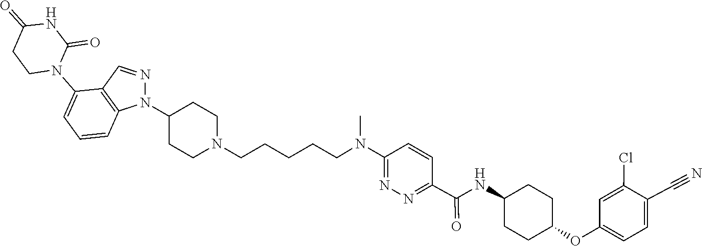 [SMILES: CN(CCCCCN1CCC(n2ncc3c(N4CCC(=O)NC4=O)cccc32)CC1)c1ccc(C(=O)N[C@H]2CC[C@H](Oc3ccc(C#N)c(Cl)c3)CC2)nn1] | Example CBL15 | MDAP: formic LCMS (formic A): Rt = 1.2 min, MH⁺ = 767 |
| | N-((1r,4r)-4-(3-chloro-4-cyanophenoxy)cyclohexyl)-6-((5-(4-(4- | | |
| | (2,4-dioxotetrahydropyrimidin-1(2H)-yl)-1H-indazol-1- | | |
| | yl)piperidin-1-yl)pentyl)(methyl)amino)pyridazine-3-carboxamide | | |
| | 12.9 mg, 19.96% | | |
| EAR221 | 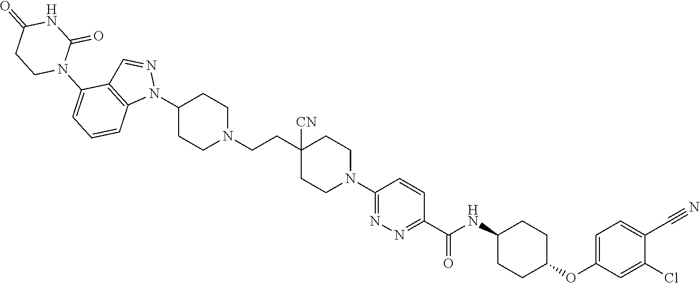 [SMILES: N#Cc1ccc(O[C@H]2CC[C@H](NC(=O)c3ccc(N4CCC(C#N)(CCN5CCC(n6ncc7c(N8CCC(=O)NC8=O)cccc76)CC5)CC4)nn3)CC2)cc1Cl] | Example CBL16 | MDAP: formic LCMS (formic A): Rt = 1.15 min, MH⁺ = 804 |
| | N-((1r,4r)-4-(3-chloro-4-cyanophenoxy)cyclohexyl)-6-(4-cyano- | | |
| | 4-(2-(4-(4-(2,4-dioxotetrahydropyrimidin-1(2H)-yl)-1H-indazol-1- | | |
| | yl)piperidin-1-yl)ethyl)piperidin-1-yl)pyridazine-3-carboxamide | | |
| | 33.1 mg, 48.9% | | |
| EAR222 | 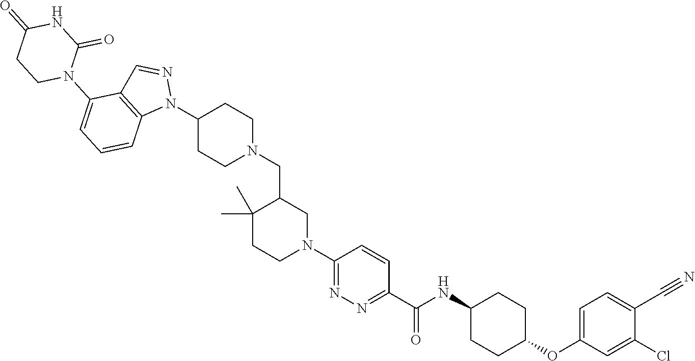 [SMILES: CC1(C)CCN(c2ccc(C(=O)N[C@H]3CC[C@H](Oc4ccc(C#N)c(Cl)c4)CC3)nn2)CC1CN1CCC(n2ncc3c(N4CCC(=O)NC4=O)cccc32)CC1] | Example CBL17 | MDAP: high pH LCMS (high pH A): Rt = 1.35 min, MH⁺ = 793 |
| | N-((1r,4r)-4-(3-chloro-4-cyanophenoxy)cyclohexyl)-6-(3-((4-(4- | | |
| | (2,4-dioxotetrahydropyrimidin-1(2H)-yl)-1H-indazol-1- | | |
| | yl)piperidin-1-yl)methyl)-4,4-dimethylpiperidin-1-yl)pyridazine-3- | | |
| | carboxamide | | |
| | 15.9 mg, 23.80% | | |
| EAR223 | 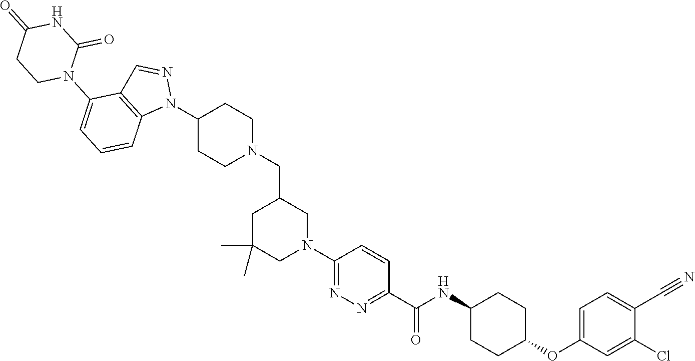 [SMILES: CC1(C)CC(CN2CCC(n3ncc4c(N5CCC(=O)NC5=O)cccc43)CC2)CN(c2ccc(C(=O)N[C@H]3CC[C@H](Oc4ccc(C#N)c(Cl)c4)CC3)nn2)C1] | Example CBL18 | MDAP: high pH LCMS (high pH A): Rt = 1.33 min, MH⁺ = 793 |
| | N-((1r,4r)-4-(3-chloro-4-cyanophenoxy)cyclohexyl)-6-(5-((4-(4- | | |
| | (2,4-dioxotetrahydropyrimidin-1(2H)-yl)-1H-indazol-1- | | |
| | yl)piperidin-1-yl)methyl)-3,3-dimethylpiperidin-1-yl)pyridazine-3- | | |
| | carboxamide | | |
| | 19.4 mg, 29.0% | | |
| EAR224 | 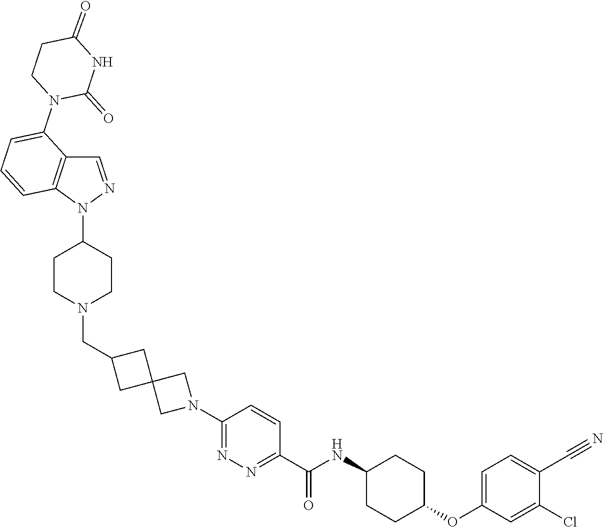 [SMILES: N#Cc1ccc(O[C@H]2CC[C@H](NC(=O)c3ccc(N4CC5(CC(CN6CCC(n7ncc8c(N9CCC(=O)NC9=O)cccc87)CC6)C5)C4)nn3)CC2)cc1Cl] | Example CBL19 | MDAP: formic LCMS (formic A): Rt = 1.17 min, MH⁺ = 777 |
| | N-((1r,4r)-4-(3-chloro-4-cyanophenoxy)cyclohexyl)-6-(6-((4-(4- | | |
| | (2,4-dioxotetrahydropyrimidin-1(2H)-yl)-1H-indazol-1- | | |
| | yl)piperidin-1-yl)methyl)-2-azaspiro[3.3]heptan-2-yl)pyridazine- | | |
| | 3-carboxamide | | |
| | 13.4 mg, 20.47% | | |
| EAR225 | 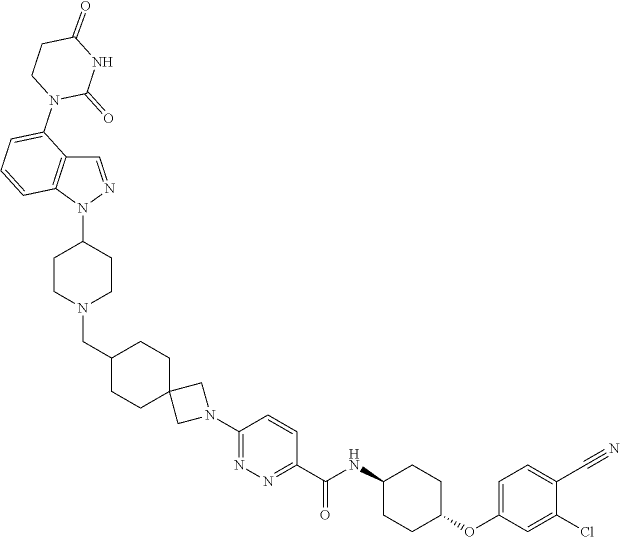 [SMILES: N#Cc1ccc(O[C@H]2CC[C@H](NC(=O)c3ccc(N4CC5(CCC(CN6CCC(n7ncc8c(N9CCC(=O)NC9=O)cccc87)CC6)CC5)C4)nn3)CC2)cc1Cl] | Example CBL20 | MDAP: high pH LCMS (high pH A): Rt = 1.27 min, MH⁺ = 805 |
| | N-((1r,4r)-4-(3-chloro-4-cyanophenoxy)cyclohexyl)-6-(7-((4-(4- | | |
| | (2,4-dioxotetrahydropyrimidin-1(2H)-yl)-1H-indazol-1- | | |
| | yl)piperidin-1-yl)methyl)-2-azaspiro[3.5]nonan-2-yl)pyridazine-3- | | |
| | carboxamide | | |
| | 21.4 mg, 31.6% | | |
| EAR226 | 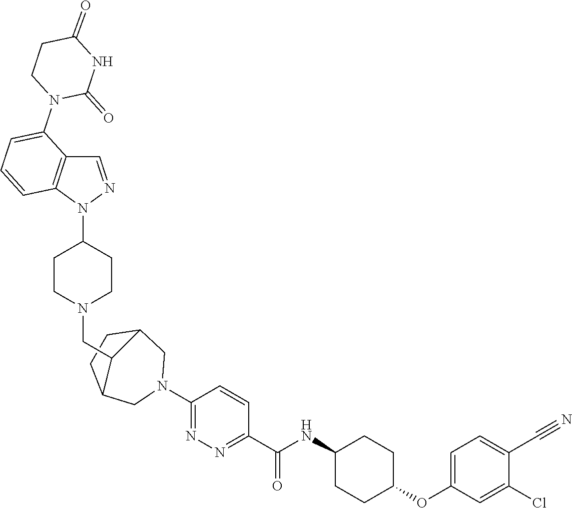 [SMILES: N#Cc1ccc(O[C@H]2CC[C@H](NC(=O)c3ccc(N4CC5CCC(C4)C5CN4CCC(n5ncc6c(N7CCC(=O)NC7=O)cccc65)CC4)nn3)CC2)cc1Cl] | Example CBL21 | MDAP: high pH LCMS (high pH A): Rt = 1.25 min, MH⁺ = 791 |
| | N-((1r,4r)-4-(3-chloro-4-cyanophenoxy)cyclohexyl)-6-(8-((4-(4- | | |
| | (2,4-dioxotetrahydropyrimidin-1(2H)-yl)-1H-indazol-1- | | |
| | yl)piperidin-1-yl)methyl)-3-azabicyclo[3.2.1]octan-3- | | |
| | yl)pyridazine-3-carboxamide | | |
| | 22.2 mg, 33.3% | | |
| EAR227 | 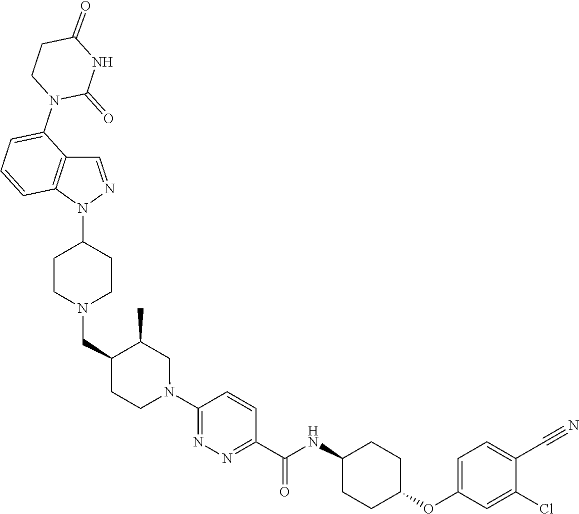 [SMILES: C[C@H]1CN(c2ccc(C(=O)N[C@H]3CC[C@H](Oc4ccc(C#N)c(Cl)c4)CC3)nn2)CC[C@H]1CN1CCC(n2ncc3c(N4CCC(=O)NC4=O)cccc32)CC1] | Example CBL22 | MDAP: high pH LCMS (high pH A): Rt = 1.25 min, MH⁺ = 779 |
| | N-((1r,4R)-4-(3-chloro-4-cyanophenoxy)cyclohexyl)-6-((3R,4R)- | | |
| | 4-((4-(4-(2,4-dioxotetrahydropyrimidin-1(2H)-yl)-1H-indazol-1- | | |
| | yl)piperidin-1-yl)methyl)-3-methylpiperidin-1-yl)pyridazine-3- | | |
| | carboxamide | | |
| | 21.6 mg, 32.9% | | |
| EAR228 | 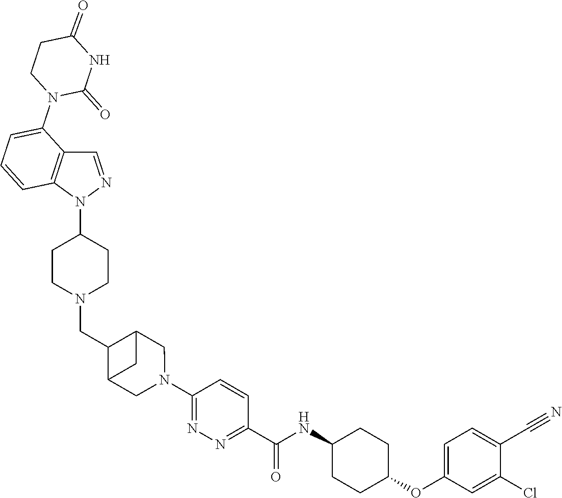 [SMILES: N#Cc1ccc(O[C@H]2CC[C@H](NC(=O)c3ccc(N4CC5CC(C4)C5CN4CCC(n5ncc6c(N7CCC(=O)NC7=O)cccc65)CC4)nn3)CC2)cc1Cl] | Example CBL23 | MDAP: formic LCMS (formic A): Rt = 1.16 min, MH⁺ = 777 |
| | N-((1r,4r)-4-(3-chloro-4-cyanophenoxy)cyclohexyl)-6-(6-((4-(4- | | |
| | (2,4-dioxotetrahydropyrimidin-1(2H)-yl)-1H-indazol-1- | | |
| | yl)piperidin-1-yl)methyl)-3-azabicyclo[3.1.1]heptan-3- | | |
| | yl)pyridazine-3-carboxamide | | |
| | 28.3 mg, 43.2% | | |

### Examples AR229-AR244

In sixteen separate reactions, to the amines (0.155 mmol) listed in the table below were added 6-chloro-N-((1r,4r)-4-(3-chloro-4-cyanophenoxy)cyclohexyl)pyridazine-3-carboxamide (may be prepared as described in Description 90; 0.039 g, 0.1 mmol) and DIPEA (0.122 mL, 0.7 mmol) dissolved in DMSO (1.25 mL). The reactions were left to stir 65° C. for 18 h, then at 80° C. for 30 min. Those reactions that were incomplete were heated to 100° C. for 1 h, then 140° C. for 45 min. Reactions were purified by MDAP (high pH) (those reactions not sufficiently pure were re-purified by MDAP [formic acid]) to give the following compounds:

| | Product structure and name | | Mass, |
|---|---|---|---|
| Example | Analytical | Amine | yield |
| EAR229 | 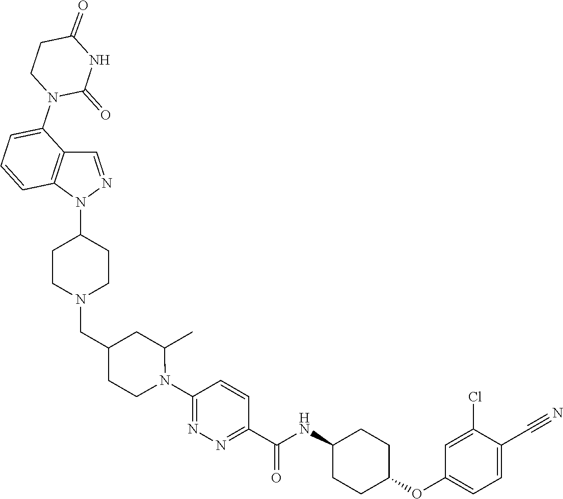 [SMILES: CC1CC(CN2CCC(n3ncc4c(N5CCC(=O)NC5=O)cccc43)CC2)CCN1c1ccc(C(=O)N[C@H]2CC[C@H](Oc3ccc(C#N)c(Cl)c3)CC2)nn1] | Example CBL24 | 7.3 mg, 8.43% |
| | N-((1r,4r)-4-(3-chloro-5-cyanophenoxy)cyclohexyl)-6-(4-((4-(4- | | |
| | (2,4-dioxotetrahydropyrimidin-1(2H)-yl)-1H-indazol-1- | | |
| | yl)piperidin-1-yl)methyl)-2-methylpiperidin-1-yl)pyridazine-3- | | |
| | carboxamide | | |
| | LCMS (formic A): Rt = 0.84 min, MH⁺ = 779 | | |
| EAR230 | 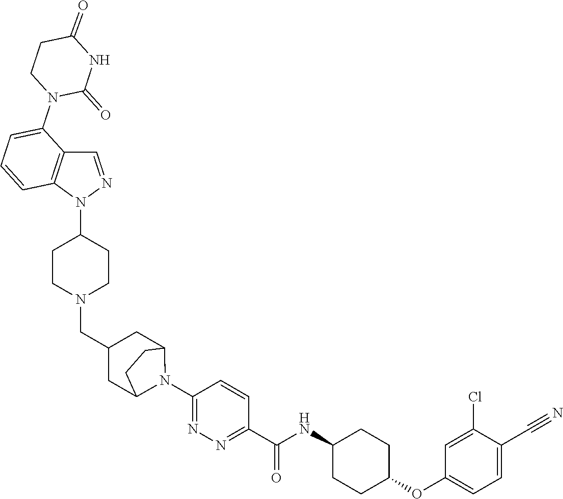 [SMILES: N#Cc1ccc(O[C@H]2CC[C@H](NC(=O)c3ccc(N4C5CCC4CC(CN4CCC(n6ncc7c(N8CCC(=O)NC8=O)cccc76)CC4)C5)nn3)CC2)cc1Cl] | Example CBL25 | 19.2 mg, 21.84% |
| | N-((1r,4r)-4-(3-chloro-4-cyanophenoxy)cyclohexyl)-6-(3-((4-(4- | | |
| | (2,4-dioxotetrahydropyrimidin-1(2H)-yl)-1H-indazol-1- | | |
| | yl)piperidin-1-yl)methyl)-8-azabicyclo[3.2.1]octan-8- | | |
| | yl)pyridazine-3-carboxamide | | |
| | LCMS (formic A): Rt = 0.79 min, MH⁺ = 791 | | |
| EAR231 | 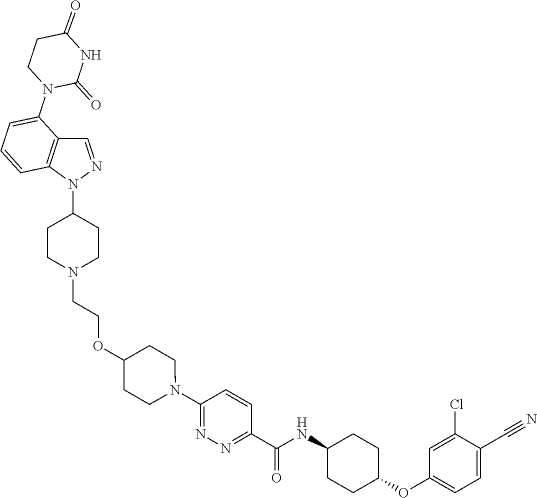 [SMILES: N#Cc1ccc(O[C@H]2CC[C@H](NC(=O)c3ccc(N4CCC(OCCN5CCC(n6ncc7c(N8CCC(=O)NC8=O)cccc76)CC5)CC4)nn3)CC2)cc1Cl] | Example CBL26 | 35 mg, 39.6% |
| | N-((1r,4r)-4-(3-chloro-4-cyanophenoxy)cyclohexyl)-6-(4-(2-(4- | | |
| | (4-(2,4-dioxotetrahydropyrimidin-1(2H)-yl)-1H-indazol-1- | | |
| | yl)piperidin-1-yl)ethoxy)piperidin-1-yl)pyridazine-3-carboxamide | | |
| | LCMS (formic A): Rt = 0.83 min, MH⁺ = 795 | | |
| EAR232 | 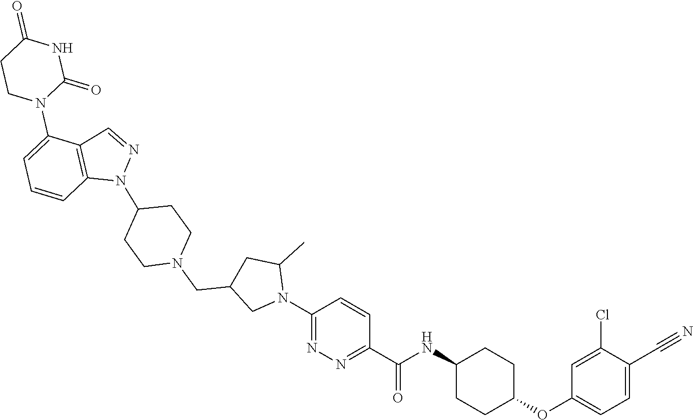 [SMILES: CC1CC(CN2CCC(n3ncc4c(N5CCC(=O)NC5=O)cccc43)CC2)CN1c1ccc(C(=O)N[C@H]2CC[C@H](Oc3ccc(C#N)c(Cl)c3)CC2)nn1] | Example CBL27 | 35.7 mg, 42% |
| | N-((1r,4r)-4-(3-chloro-4-cyanophenoxy)cyclohexyl)-6-(4-((4-(4- | | |
| | (2,4-dioxotetrahydropyrimidin-1(2H)-yl)-1H-indazol-1- | | |
| | yl)piperidin-1-yl)methyl)-2-methylpyrrolidin-1-yl)pyridazine-3- | | |
| | carboxamide | | |
| | LCMS (formic A): Rt = 0.81 min, MH⁺ = 765 | | |
| EAR233 | 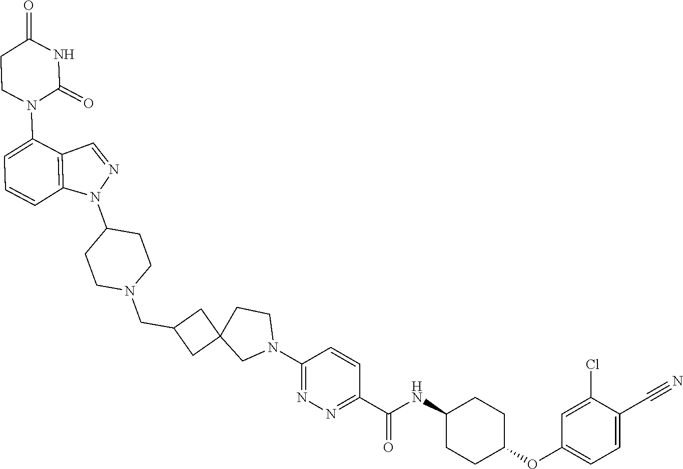 [SMILES: N#Cc1ccc(O[C@H]2CC[C@H](NC(=O)c3ccc(N4CCC5(CC(CN6CCC(n7ncc8c(N9CCC(=O)NC9=O)cccc87)CC6)C5)C4)nn3)CC2)cc1Cl] | Example CBL28 | 20 mg, 22.75% |
| | N-((1r,4r)-4-(3-chloro-4-cyanophenoxy)cyclohexyl)-6-(2-((4-(4- | | |
| | (2,4-dioxotetrahydropyrimidin-1(2H)-yl)-1H-indazol-1- | | |
| | yl)piperidin-1-yl)methyl)-6-azaspiro[3.4]octan-6-yl)pyridazine-3- | | |
| | carboxamide | | |
| | LCMS (formic A): Rt = 0.80 min, MH⁺ = 791 | | |
| EAR234 | 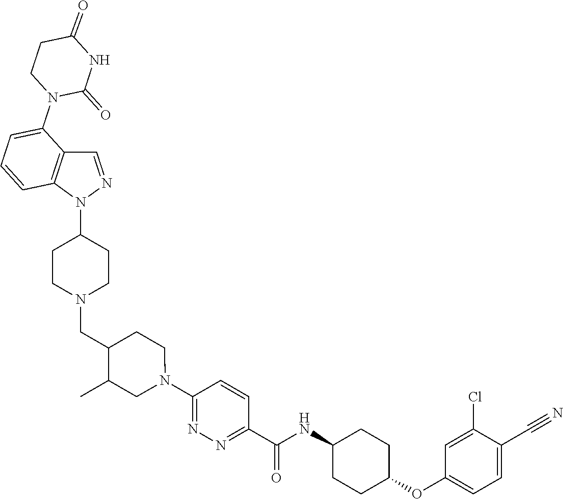 [SMILES: CC1CN(c2ccc(C(=O)N[C@H]3CC[C@H](Oc4ccc(C#N)c(Cl)c4)CC3)nn2)CCC1CN1CCC(n2ncc3c(N4CCC(=O)NC4=O)cccc32)CC1] | Example CBL29 | 36.4 mg, 42.0% |
| | N-((1r,4r)-4-(3-chloro-4-cyanophenoxy)cyclohexyl)-6-(4-((4-(4- | | |
| | (2,4-dioxotetrahydropyrimidin-1(2H)-yl)-1H-indazol-1- | | |
| | yl)piperidin-1-yl)methyl)-3-methylpiperidin-1-yl)pyridazine-3- | | |
| | carboxamide | | |
| | LCMS (formic A): Rt = 0.79 min, MH⁺ = 779 | | |
| EAR235 | 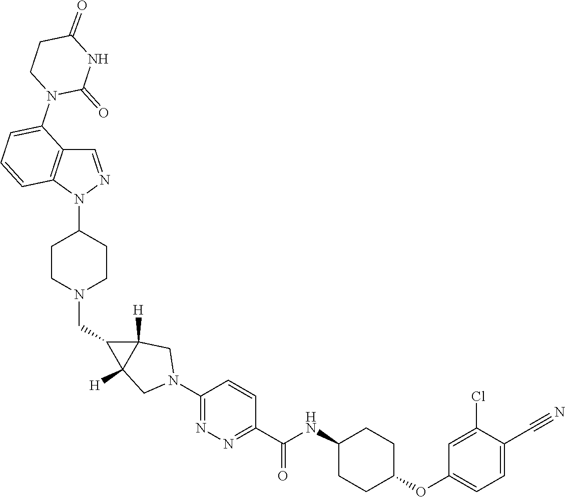 [SMILES: N#Cc1ccc(O[C@H]2CC[C@H](NC(=O)c3ccc(N4C[C@@H]5[C@H](CN6CCC(n7ncc8c(N9CCC(=O)NC9=O)cccc87)CC6)[C@@H]5C4)nn3)CC2)cc1Cl] | Example CBL30 | 30 mg, 35.4% |
| | N-((1r,4R)-4-(3-chloro-4-cyanophenoxy)cyclohexyl)-6- | | |
| | ((1R,5S,6R)-6-((4-(4-(2,4-dioxotetrahydropyrimidin-1(2H)-yl)- | | |
| | 1H-indazol-1-yl)piperidin-1-yl)methyl)-3-azabicyclo[3.1.0]hexan- | | |
| | 3-yl)pyridazine-3-carboxamide | | |
| | LCMS (formic A): Rt = 0.80 min, MH⁺ = 763 | | |
| EAR236 | 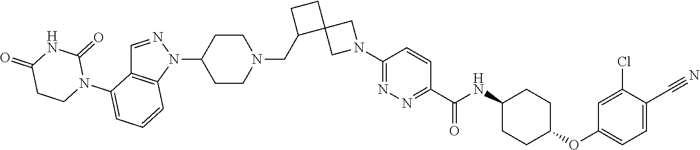 [SMILES: N#Cc1ccc(O[C@H]2CC[C@H](NC(=O)c3ccc(N4CC5(CCC5CN5CCC(n6ncc7c(N8CCC(=O)NC8=O)cccc76)CC5)C4)nn3)CC2)cc1Cl] | Example CBL31 | 13.9 mg, 16.09% |
| | N-((1r,4r)-4-(3-chloro-4-cyanophenoxy)cyclohexyl)-6-(5-((4-(4- | | |
| | (2,4-dioxotetrahydropyrimidin-1(2H)-yl)-1H-indazol-1- | | |
| | yl)piperidin-1-yl)methyl)-2-azaspiro[3.3]heptan-2-yl)pyridazine- | | |
| | 3-carboxamide | | |
| | LCMS (formic A): Rt = 0.83 min, MH⁺ = 777 | | |
| EAR237 | 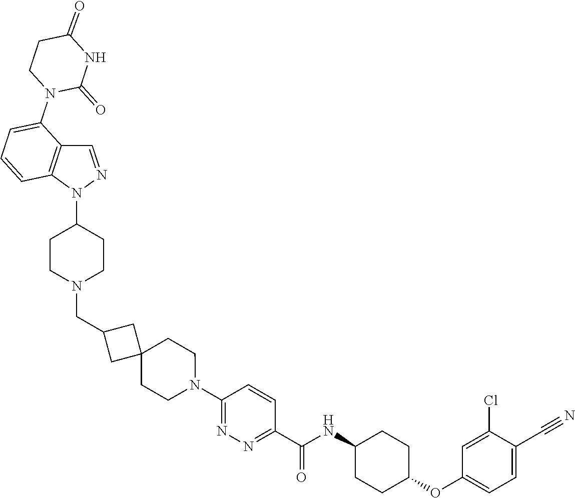 [SMILES: N#Cc1ccc(O[C@H]2CC[C@H](NC(=O)c3ccc(N4CCC5(CC4)CC(CN4CCC(n6ncc7c(N8CCC(=O)NC8=O)cccc76)CC4)C5)nn3)CC2)cc1Cl] | Example CBL32 | 4.9 mg, 5.48% |
| | N-((1r,4r)-4-(3-chloro-4-cyanophenoxy)cyclohexyl)-6-(2-((4-(4- | | |
| | (2,4-dioxotetrahydropyrimidin-1(2H)-yl)-1H-indazol-1- | | |
| | yl)piperidin-1-yl)methyl)-7-azaspiro[3.5]nonan-7-yl)pyridazine-3- | | |
| | carboxamide | | |
| | LCMS (formic A): Rt = 0.81 min, MH⁺ = 805 | | |
| EAR238 | 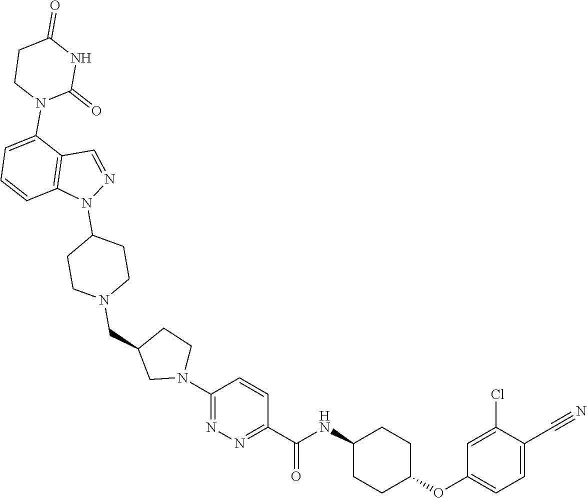 [SMILES: N#Cc1ccc(O[C@H]2CC[C@H](NC(=O)c3ccc(N4CC[C@H](CN5CCC(n6ncc7c(N8CCC(=O)NC8=O)cccc76)CC5)C4)nn3)CC2)cc1Cl] | Example CBL33 | 37 mg, 44.3% |
| | N-((1r,4R)-4-(3-chloro-4-cyanophenoxy)cyclohexyl)-6-((R)-3-((4- | | |
| | (4-(2,4-dioxotetrahydropyrimidin-1(2H)-yl)-1H-indazol-1- | | |
| | yl)piperidin-1-yl)methyl)pyrrolidin-1-yl)pyridazine-3-carboxamide | | |
| | LCMS (formic A): Rt = 0.73 min, MH⁺ = 751 | | |
| EAR239 | 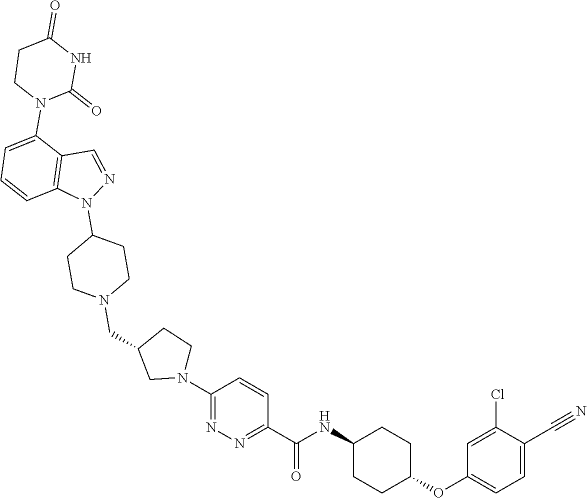 [SMILES: N#Cc1ccc(O[C@H]2CC[C@H](NC(=O)c3ccc(N4CC[C@@H](CN5CCC(n6ncc7c(N8CCC(=O)NC8=O)cccc76)CC5)C4)nn3)CC2)cc1Cl] | Example CBL34 | 39 mg, 46.7% |
| | N-((1r,4R)-4-(3-chloro-4-cyanophenoxy)cyclohexyl)-6-((S)-3-((4- | | |
| | (4-(2,4-dioxotetrahydropyrimidin-1(2H)-yl)-1H-indazol-1- | | |
| | yl)piperidin-1-yl)methyl)pyrrolidin-1-yl)pyridazine-3-carboxamide | | |
| | LCMS (formic A): Rt = 0.73 min, MH⁺ = 751 | | |
| EAR240 | 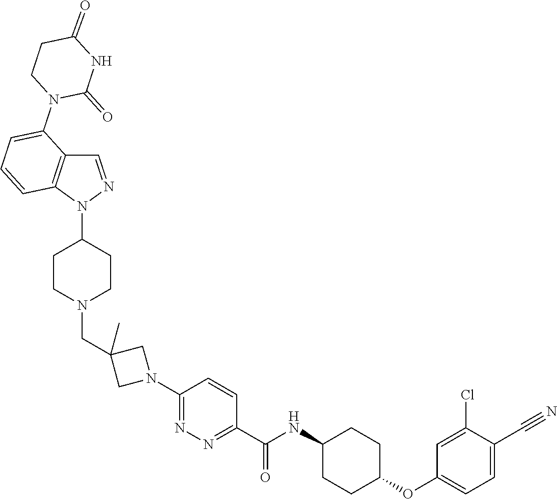 [SMILES: CC1(CN2CCC(n3ncc4c(N5CCC(=O)NC5=O)cccc43)CC2)CN(c2ccc(C(=O)N[C@H]3CC[C@H](Oc4ccc(C#N)c(Cl)c4)CC3)nn2)C1] | Example CBL35 | 26.9 mg, 32.2% |
| | N-((1r,4r)-4-(3-chloro-4-cyanophenoxy)cyclohexyl)-6-(3-((4-(4- | | |
| | (2,4-dioxotetrahydropyrimidin-1(2H)-yl)-1H-indazol-1- | | |
| | yl)piperidin-1-yl)methyl)-3-methylazetidin-1-yl)pyridazine-3- | | |
| | carboxamide | | |
| | LCMS (formic A): Rt = 0.74 min, MH⁺ = 751 | | |
| EAR241 | 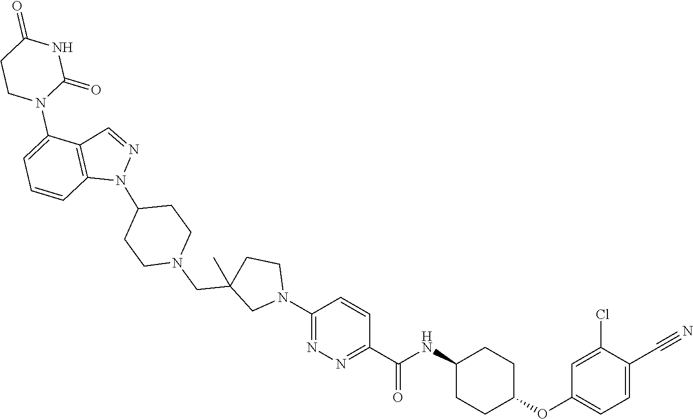 [SMILES: CC1(CN2CCC(n3ncc4c(N5CCC(=O)NC5=O)cccc43)CC2)CCN(c2ccc(C(=O)N[C@H]3CC[C@H](Oc4ccc(C#N)c(Cl)c4)CC3)nn2)C1] | Example CBL36 | 17.6 mg, 20.70% |
| | N-((1r,4r)-4-(3-chloro-4-cyanophenoxy)cyclohexyl)-6-(3-((4-(4- | | |
| | (2,4-dioxotetrahydropyrimidin-1(2H)-yl)-1H-indazol-1- | | |
| | yl)piperidin-1-yl)methyl)-3-methylpyrrolidin-1-yl)pyridazine-3- | | |
| | carboxamide | | |
| | LCMS (formic A): Rt = 0.75 min, MH⁺ = 765 | | |
| EAR242 | 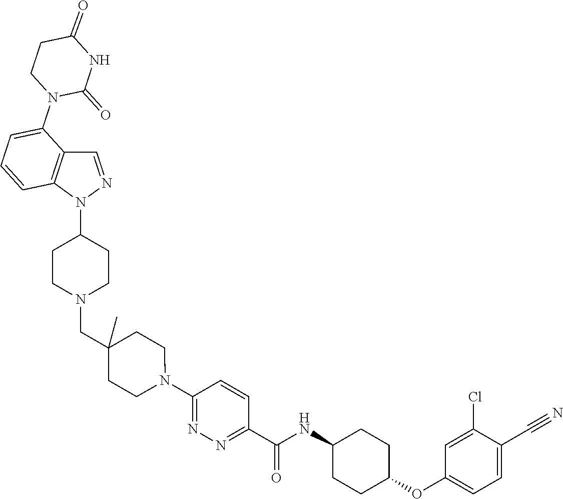 [SMILES: CC1(CN2CCC(n3ncc4c(N5CCC(=O)NC5=O)cccc43)CC2)CCN(c2ccc(C(=O)N[C@H]3CC[C@H](Oc4ccc(C#N)c(Cl)c4)CC3)nn2)CC1] | Example CBL37 | 6.7 mg, 7.74% |
| | N-((1r,4r)-4-(3-chloro-4-cyanophenoxy)cyclohexyl)-6-(4-((4-(4- | | |
| | (2,4-dioxotetrahydropyrimidin-1(2H)-yl)-1H-indazol-1- | | |
| | yl)piperidin-1-yl)methyl)-4-methylpiperidin-1-yl)pyridazine-3- | | |
| | carboxamide | | |
| | LCMS (formic A): Rt = 0.78 min, MH⁺ = 779 | | |
| EAR243 | 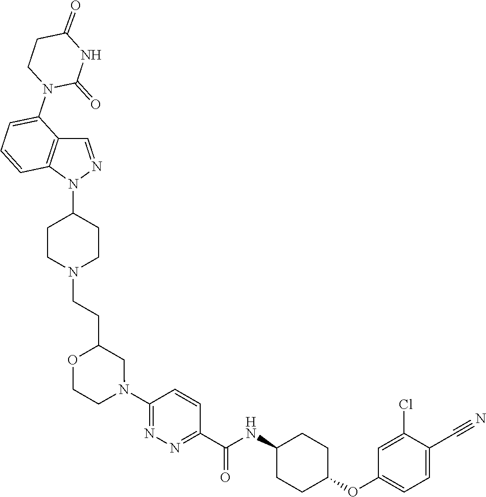 [SMILES: N#Cc1ccc(O[C@H]2CC[C@H](NC(=O)c3ccc(N4CCOC(CCN5CCC(n6ncc7c(N8CCC(=O)NC8=O)cccc76)CC5)C4)nn3)CC2)cc1Cl] | Example CBL38 | 38.7 mg, 44.6% |
| | N-((1r,4r)-4-(3-chloro-4-cyanophenoxy)cyclohexyl)-6-(2-(2-(4- | | |
| | (4-(2,4-dioxotetrahydropyrimidin-1(2H)-yl)-1H-indazol-1- | | |
| | yl)piperidin-1-yl)ethyl)morpholino)pyridazine-3-carboxamide | | |
| | LCMS (formic A): Rt = 0.77 min, MH⁺ = 781 | | |
| EAR244 | 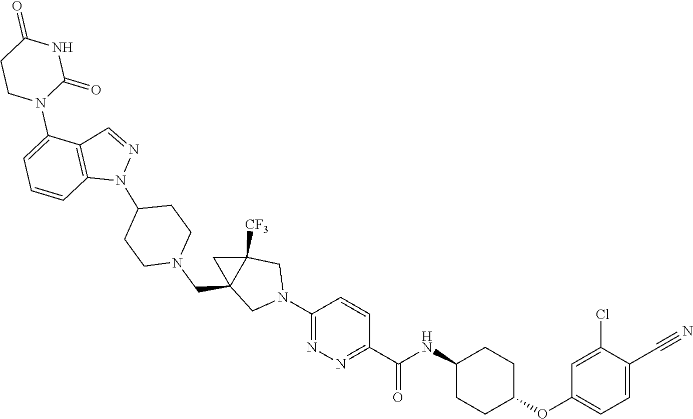 [SMILES: N#Cc1ccc(O[C@H]2CC[C@H](NC(=O)c3ccc(N4C[C@]5(CN6CCC(n7ncc8c(N9CCC(=O)NC9=O)cccc87)CC6)C[C@]5(C(F)(F)F)C4)nn3)CC2)cc1Cl] | Example CBL39 | 13.3 mg, 14.40% |
| | N-((1r,4R)-4-(3-chloro-4-cyanophenoxy)cyclohexyl)-6-((1R,5R)- | | |
| | 1-((4-(4-(2,4-dioxotetrahydropyrimidin-1(2H)-yl)-1H-indazol-1- | | |
| | yl)piperidin-1-yl)methyl)-5-(trifluoromethyl)-3- | | |
| | azabicyclo[3.1.0]hexan-3-yl)pyridazine-3-carboxamide | | |
| | LCMS (formic A): Rt = 0.83 min, MH⁺ = 831 | | |

### Example 245

### (2S,5R)-4-(3-Chloro-4-cyanophenyl)-N-(6-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyridin-3-yl)-2,5-dimethylpiperazine-1-carboxamide (EAR245)

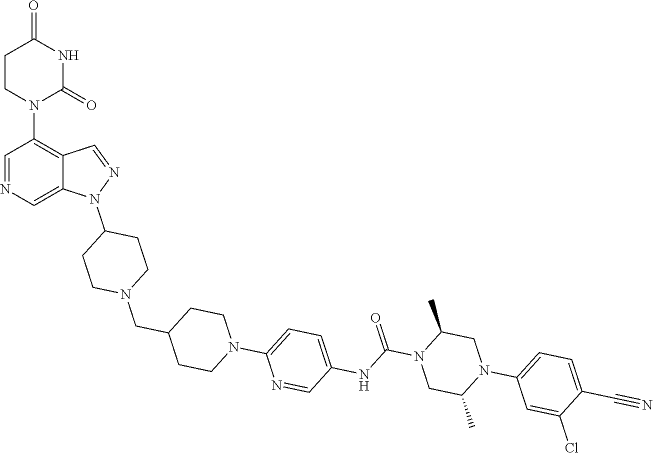 [SMILES: C[C@@H]1CN(C(=O)Nc2ccc(N3CCC(CN4CCC(n5ncc6c(N7CCC(=O)NC7=O)cncc65)CC4)CC3)nc2)[C@@H](C)CN1c1ccc(C#N)c(Cl)c1]

1-(1-(Piperidin-4-yl)-1H-pyrazolo[3,4-c]pyridin-4-yl)dihydropyrimidine-2,4(1H,3H)-dione (may be prepared as described in Example ECB8; 35 mg, 0.111 mmol) and (2S,5R)-4-(3-chloro-4-cyanophenyl)-N-(6-(4-formylpiperidin-1-yl)pyridin-3-yl)-2,5-dimethylpiperazine-1-carboxamide (may be prepared as described in Description 209; 78 mg, 0.162 mmol) were stirred in DCM (6 mL) at room temperature for 0.5 h. Sodium triacetoxyborohydride (451 mg, 2.128 mmol) was added, and after 2.5 h the reaction was partitioned between DCM (25 mL) and saturated sodium hydrogen carbonate (25 mL). The organic phase was separated, and the aqueous phase was extracted with DCM (10 mL×2). The combined organic phases were washed with water (30 mL) and brine (30 mL), dried, concentrated and purified by MDAP (high pH) to give (2S,5R)-4-(3-chloro-4-cyanophenyl)-N-(6-(4-((4-(4-(2,4-dioxotetrahydropyrimidin-1(2H)-yl)-1H-pyrazolo[3,4-c]pyridin-1-yl)piperidin-1-yl)methyl)piperidin-1-yl)pyridin-3-yl)-2,5-dimethylpiperazine-1-carboxamide (15.3 mg, 0.020 mmol, 17.63% yield). LCMS (high pH A): Rt=1.07 min, MH⁺=779.5

### Example AR246

### N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-indazol-1-yl)piperidin-1-yl)methyl)piperidin-1-yl)pyridazine-3-carboxamide (EAR246)

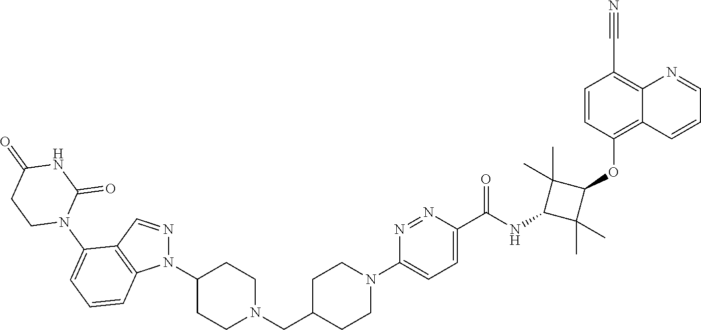 [SMILES: CC1(C)[C@H](NC(=O)c2ccc(N3CCC(CN4CCC(n5ncc6c(N7CCC(=O)NC7=O)cccc65)CC4)CC3)nn2)C(C)(C)[C@H]1Oc1ccc(C#N)c2ncccc12]

A solution of 1-(1-(piperidin-4-yl)-1H-indazol-4-yl)dihydropyrimidine-2,4(1H,3H)-dione (may be prepared as described in Example ECB4, 76.4 mg, 1 equivalent, 244 μmol) and N-((1r,3r)-3-((8-cyanoquinolin-5-yl)oxy)-2,2,4,4-tetramethylcyclobutyl)-6-(4-formylpiperidin-1-yl)pyridazine-3-carboxamide (may be prepared as described in Description 345; 125 mg, 244 μmol) in DCM (6 mL) and methanol (1 mL) was stirred at room temperature for 30 min. Sodium triacetoxyborohydride (155 mg, 732 μmol) was added and the mixture was stirred at room temperature for 16 h. The mixture was diluted with DCM and washed sequentially with water and brine. The organic layer was dried over Na₂SO₄, filtered and concentrated in vacuo. The sample triturated with methanol, then the resulting solid was dried to give the desired product (145 mg, 70% yield) as a white solid. LCMS (formic A): Rt=0.92 min, MH⁺=810.

### Example AR247

### N-((1r,3r)-3-(3-Chloro-4-cyanophenoxy)-2,2,4,4-tetramethylcyclobutyl)-6-(4-((4-(4-(2,4-dioxotetrahydropyrimidin-1(2H)-yl)-7H-pyrrolo[2,3-d]pyrimidin-7-yl)piperidin-1-yl)methyl)piperidin-1-yl)pyridazine-3-carboxamide (EAR247)

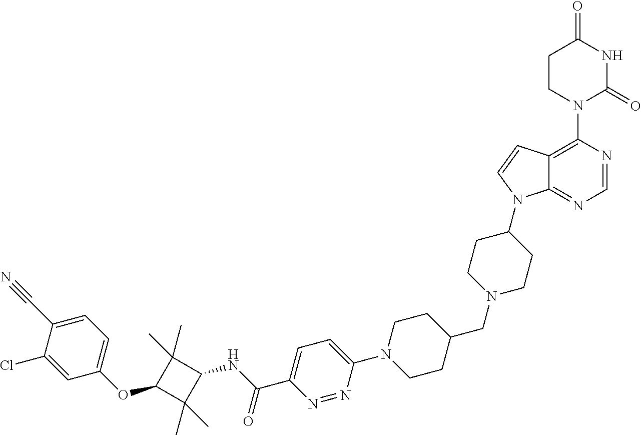 [SMILES: CC1(C)[C@H](NC(=O)c2ccc(N3CCC(CN4CCC(n5ccc6c(N7CCC(=O)NC7=O)ncnc65)CC4)CC3)nn2)C(C)(C)[C@H]1Oc1ccc(C#N)c(Cl)c1]

To a stirred solution of N-((1r,3r)-3-(3-chloro-4-cyanophenoxy)-2,2,4,4-tetramethylcyclobutyl)-6-(4-formylpiperidin-1-yl)pyridazine-3-carboxamide (may be prepared as described in Description 347; 226 mg, 456 μmol) in THF (5.00 mL) and methanol (1.00 mL) was added 1-(7-(piperidin-4-yl)-7H-pyrrolo[2,3-d]pyrimidin-4-yl)dihydropyrimidine-2,4(1H,3H)-dione (143 mg, 1 equivalents, 456 μmol) and sodium triacetoxyborohydride (290 mg, 3 equivalents, 1.37 mmol) then the mixture stirred at rt for 16 h. Further THF (5.00 mL) and sodium triacetoxyborohydride (290 mg, 3 equivalents, 1.37 mmol) were added then the mixture was stirred at room temperature for 24 h. The mixture was diluted with ethyl acetate (25 mL) and water (50 mL), then the phases were separated. The aqueous layer was back-extracted with ethyl acetate (2×25 mL), then the organic layers were combined, dried using a hydrophobic frit and evaporated in vacuo. The sample was purified by MDAP (high pH), then the appropriate fractions were combined and dried under a stream of nitrogen to give the desired product (68.1 mg, 19% yield) as a white solid. LCMS (high pH A): Rt=1.40 min, MH⁺=794.

### Example AR248

### N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-indazol-1-yl)piperidin-1-yl)methyl)piperidin-1-yl)pyridazine-3-carboxamide (EAR248)

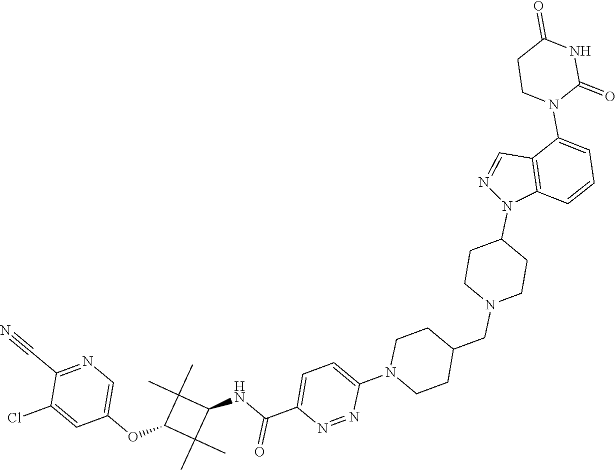 [SMILES: CC1(C)[C@H](NC(=O)c2ccc(N3CCC(CN4CCC(n5ncc6c(N7CCC(=O)NC7=O)cccc65)CC4)CC3)nn2)C(C)(C)[C@H]1Oc1cnc(C#N)c(Cl)c1]

PCC (77.8 mg, 361 μmol) and silica (technical grade, pore size 60 Å, 230-400 mesh particle size, 40-63 μm particle size 86.7 mg, 1.44 mmol) were suspended in DCM (5.0 mL) under nitrogen. N-((1r,3r)-3-((5-Chloro-6-cyanopyridin-3-yl)oxy)-2,2,4,4-tetramethylcyclobutyl)-6-(4-(hydroxymethyl)piperidin-1-yl)pyridazine-3-carboxamide (may be prepared as described in Description 350; 90.0 mg, 180 μmol) was added in one then the mixture was stirred at room temperature for 2 h. The mixture was filtered through a pad of celite and concentrated under reduced pressure. The mixture was dissolved in DCM (5.0 mL), then 1-(1-(piperidin-4-yl)-1H-indazol-4-yl)dihydropyrimidine-2,4(1H,3H)-dione (may be prepared as described in Example CB18; 56.5 mg, 180 μmol) was added. Sodium triacetoxyborohydride (76.5 mg, 361 μmol) was added then the mixture was stirred at room temperature for 18 hours. The reaction was treated with saturated NaHCO₃ (saturated aqueous) and extracted with DCM. The organic layers were combined, dried over anhydrous Na₂SO₄, filtered and concentrated in vacuo. The sample was dissolved in methanol:DMSO 3:1 and purified by preparative HPLC (XBridge 30 mm×100 mm) using a 40-100% acetonitrile-water (+0.1% ammonium bicarbonate modifier) gradient over 15 min (flow rate 40 mL/min). The appropriate fractions were combined and evaporated in vacuo to give the desired product (32.4 mg, 21% yield) as a white solid. LCMS (formic A) Rt=0.98 min, MH⁺=794.

### Example AR249

### N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-indazol-1-yl)piperidin-1-yl)methyl)piperidin-1-yl)pyridazine-3-carboxamide hydrochloride (EAR249)

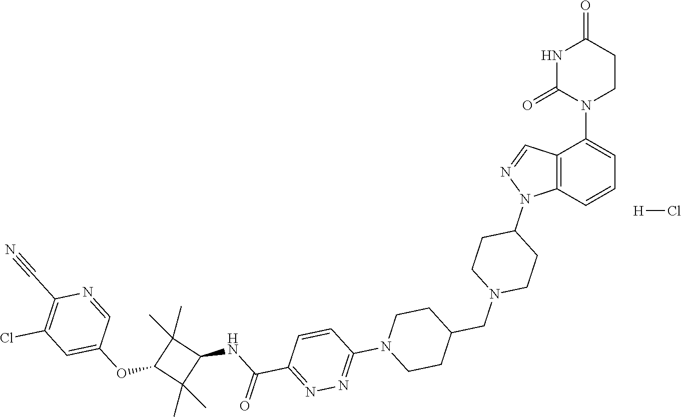

To a solution of N-((1r,3r)-3-((5-chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-indazol-1-yl)piperidin-1-yl)methyl)piperidin-1-yl)pyridazine-3-carboxamide (may be prepared as described in Example AR248; 31.0 mg, 39.0 μmol) in DCM (1.00 mL) and methanol (0.50 mL) was added HCl (3M in CPME, 13.0 μL, 39.0 μmol) then the mixture was stirred at room temperature for 1 hour. The reaction mixture was concentrated under a stream of nitrogen to give the desired product (32 mg, 94% yield) as a white solid. LCMS (formic A) Rt=0.98 min, MH⁺=794.

### Example AR250

### N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyridazine-3-carboxamide (EAR250)

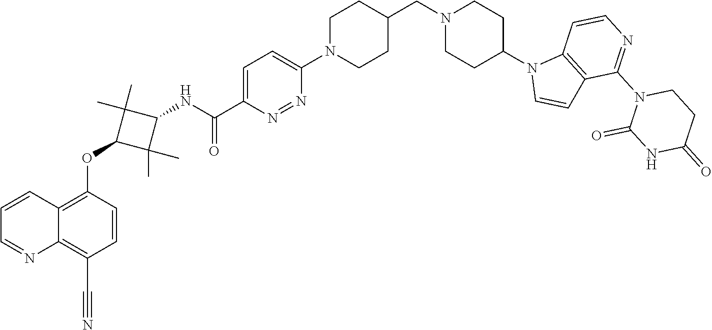 [SMILES: CC1(C)[C@H](NC(=O)c2ccc(N3CCC(CN4CCC(n5ccc6c(N7CCC(=O)NC7=O)nccc65)CC4)CC3)nn2)C(C)(C)[C@H]1Oc1ccc(C#N)c2ncccc12]

To a suspension of N-((1r,3r)-3-((8-cyanoquinolin-5-yl)oxy)-2,2,4,4-tetramethylcyclobutyl)-6-(4-formylpiperidin-1-yl)pyridazine-3-carboxamide (may be prepared as described in Description 345, 93.0 mg, 181 μmol) and 1-(1-(piperidin-4-yl)-1H-pyrrolo[3,2-c]pyridin-4-yl)dihydropyrimidine-2,4(1H,3H)-dione (may be prepared as described in Example CB25, 56.9 mg, 181 μmol) in DCM (4.00 mL) and methanol (1.0 mL) was added sodium triacetoxyborohydride (115 mg, 544 μmol) and the mixture was stirred at room temperature for 1.5 h. The reaction mixture was diluted with DCM and the organic layer was washed sequentially with water and brine. The organic layer was dried over Na₂SO₄, filtered and concentrated in vacuo. The sample was purified by preparative HPLC (XBridge 30 mm×100 mm) using a 40-100% acetonitrile-water (+0.05% ammonium bicarbonate modifier) gradient over 15 min (flow rate 40 mL/min). The appropriate fractions were combined and evaporated in vacuo to give the desired product (58 mg, 38% yield) as a white solid. LCMS (formic A) Rt=0.86 min, MH⁺=810.

### Example AR251

### N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyridazine-3-carboxamide hydrochloride (EAR251)

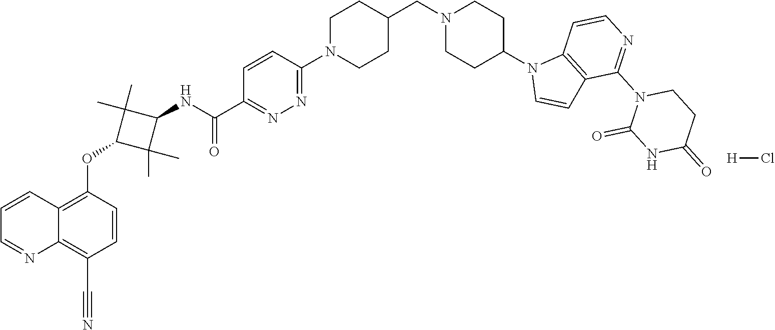

To a solution of N-((1r,3r)-3-((8-cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)pyridazine-3-carboxamide (may be prepared as described in Example AR250; 56.0 mg, 69.1 μmol) in DCM (1.00 mL) and methanol (0.50 mL) was added HCl (3M in CPME, 23.0 μL, 69.1 μmol) and it was stirred at room temperature for 1 h. The reaction mixture was concentrated under a stream of nitrogen to give the desired product (58 mg, 94% yield) as a white solid. LCMS (formic A) Rt=0.86 min, MH⁺=810.

### Example AR252

### N-((1r,3r)-3-((5-Chloro-6-cyanopyridin-3-yl)oxy)-2,2,4,4-tetramethylcyclobutyl)-2-(4-((4-(4-(2,4-dioxotetrahydropyrimidin-1(2H)-yl)-1H-indol-1-yl)piperidin-1-yl)methyl)piperidin-1-yl)pyrimidine-5-carboxamide (EAR252)

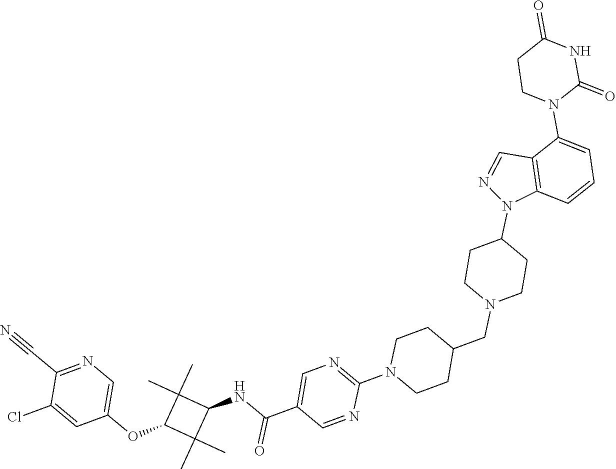 [SMILES: CC1(C)[C@H](NC(=O)c2cnc(N3CCC(CN4CCC(n5ncc6c(N7CCC(=O)NC7=O)cccc65)CC4)CC3)nc2)C(C)(C)[C@H]1Oc1cnc(C#N)c(Cl)c1]

PCC (17.3 mg, 80.2 μmol) and silica (technical grade, 20.0 mg, 333 μmol) were suspended in DCM (1.0 mL), then N-((1r,3r)-3-((5-chloro-6-cyanopyridin-3-yl)oxy)-2,2,4,4-tetramethylcyclobutyl)-2-(4-(hydroxymethyl)piperidin-1-yl)pyrimidine-5-carboxamide (may be prepared as described in Description 351; 20.0 mg, 40.1 μmol) then the mixture was stirred at room temperature for 90 min. The reaction mixture was filtered through a pad of Celite, washing with DCM, then the resulting solution was concentrated in vacuo. The sample was dissolved in MeCN (1.0 mL), then 1-(1-(piperidin-4-yl)-1H-indol-4-yl)dihydropyrimidine-2,4(1H,3H)-dione (may be prepared as described in Example CB1, 12.5 mg, 40.1 μmol) then sodium triacetoxyborohydride (17.0 mg, 80.2 μmol) was added then the mixture was stirred at room temperature for 2 h. The mixture was treated with NaHCO₃ (5% aq., 5 mL) and extracted with 9:1 DCM:methanol (2×20 mL). The combined organic layers were dried over anhydrous Na₂SO₄, filtered and concentrated in vacuo. The sample was purified by preparative HPLC (XBridge 19 mm×150 mm) using a 50-80% acetonitrile-water (+0.05% ammonium bicarbonate modifier) gradient over 15 min (flow rate 20 mL/min). The appropriate fractions were combined and evaporated in vacuo to give the desired product (12 mg, 36% yield) as a white solid. LCMS (formic A): Rt=0.98 min, MH⁺=794.

### Example AR253

### N-((1r,3r)-3-((5-Chloro-6-cyanopyridin-3-yl)oxy)-2,2,4,4-tetramethylcyclobutyl)-2-(4-((4-(4-(2,4-dioxotetrahydropyrimidin-1(2H)-yl)-1H-indazol-1-yl)piperidin-1-yl)methyl)piperidin-1-yl)pyrimidine-5-carboxamide hydrochloride (EAR253)

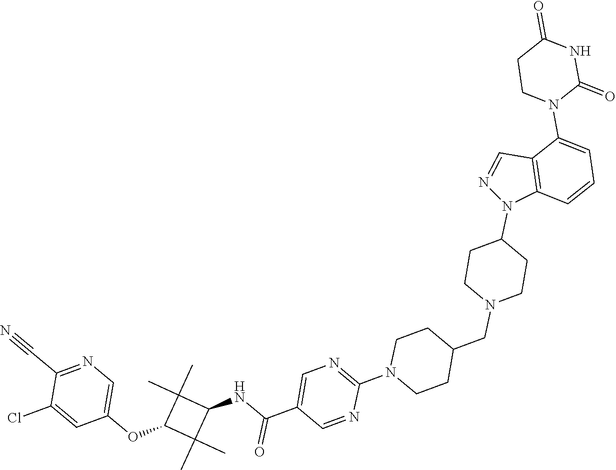 [SMILES: CC1(C)[C@H](NC(=O)c2cnc(N3CCC(CN4CCC(n5ncc6c(N7CCC(=O)NC7=O)cccc65)CC4)CC3)nc2)C(C)(C)[C@H]1Oc1cnc(C#N)c(Cl)c1]

N-((1r,3r)-3-((5-Chloro-6-cyanopyridin-3-yl)oxy)-2,2,4,4-tetramethylcyclobutyl)-2-(4-(hydroxymethyl)piperidin-1-yl)pyrimidine-5-carboxamide (may be prepared as described in Description 351, 114 mg, 0.228 mmol) was dissolved in DCM (5 mL). PCC (98.5 mg, 457 μmol) and SiO₂ (100 mg, 1.66 mmol) were added and the mixture was stirred at room temperature for 4 h. The mixture was diluted with DCM (5 mL) and directly purified by chromatography on Si (10 g) 95:5 DCM:methanol. The appropriate fractions were combined and concentrated in vacuo, then the sample was directly dissolved in MeCN (2 mL). 1-(1-(Piperidin-4-yl)-1H-indazol-4-yl)dihydropyrimidine-2,4(1H,3H)-dione, trifluoroacetic acid salt (may be prepared as described in Example CB4, 57.7 mg, 0.1350 mmol) was followed by sodium triacetoxyborohydride (96.8 mg, 457 μmol) then the mixture was stirred at room temperature for 4 h. The mixture was treated with Na₂CO₃ (5% aq., 5 mL) and the resulting mixture was extracted with DCM:methanol 9:1 (3×20 mL). The organic layers were combined, dried over anhydrous Na₂SO₄, filtered and concentrated under reduced pressure. The sample was purified by preparative HPLC (XBridge 30 mm×100 mm) using a 50-80% acetonitrile-water (modified with 0.05% ammonium bicarbonate and aqueous ammonia to pH 10) gradient over 15 min (flow rate 40 ml/min). The appropriate fractions were combined and evaporated in vacuo, then the sample was was dissolved in DCM:methanol (9:1, 15 mL), treated with HCl in isopropanol (0.6M, 126 μL, 0.0756 mmol) and stirred for 1 h. The solution was concentrated in vacuo to give the desired product (52.5 mg, 26% yield) as a white solid. LCMS (formic A): Rt=0.98 min, MH⁺=794.

### Examples AR254-AR259

### General Procedure

A mixture of N-((1r,3r)-3-((5-chloro-6-cyanopyridin-3-yl)oxy)-2,2,4,4-tetramethylcyclobutyl)-6-(4-formylpiperidin-1-yl)nicotinamide (may be prepared as described in Description 353, 99.2 mg, 0.200 mmol), the appropriate amine (200 μmol) in DCM (3 mL) and methanol (0.5 mL) was stirred at room temperature for 1 h. Sodium triacetoxyborohydride (600 μmol) was added and the mixture was stirred at room temperature for 16 h. The mixture was diluted with DCM and washed sequentially with water and brine. The organic layer was dried over Na₂SO₄, filtered and concentrated. The sample was purified by preparative HPLC (acetonitrile-water (+0.05% ammonium bicarbonate adjusted to pH 10 with aqueous ammonia)), then the appropriate fractions were combined and evaporated in vacuo to give the desired product.

| | Name | | Mass, |
|---|---|---|---|
| Example | Analytical | Amine | yield |
| EAR254 | 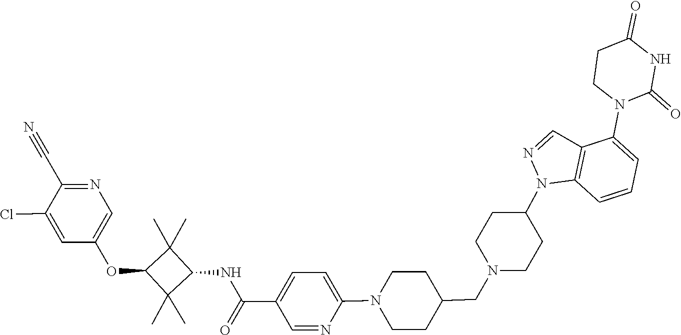 [SMILES: CC1(C)[C@H](NC(=O)c2ccc(N3CCC(CN4CCC(n5ncc6c(N7CCC(=O)NC7=O)cccc65)CC4)CC3)nc2)C(C)(C)[C@H]1Oc1cnc(C#N)c(Cl)c1] | CB4 | 94 mg, 70% yield |
| | N-((1r,3r)-3-((5-Chloro-6-cyanopyridin-3-yl)oxy)-2,2,4,4- | | |
| | tetramethylcyclobutyl)-6-(4-((4-(4-(2,4- | | |
| | dioxotetrahydropyrimidin-1(2H)-yl)-1H-indazol-1-yl)piperidin- | | |
| | 1-yl)methyl)piperidin-1-yl)nicotinamide | | |
| | LCMS (formic A): Rt = 0.94 min, MH⁺ = 793. | | |
| EAR255 | 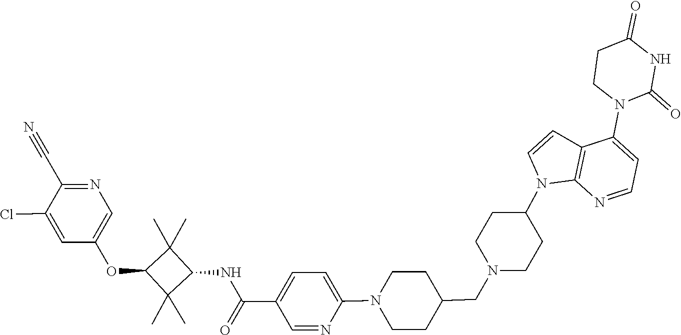 [SMILES: CC1(C)[C@H](NC(=O)c2ccc(N3CCC(CN4CCC(n5ccc6c(N7CCC(=O)NC7=O)ccnc65)CC4)CC3)nc2)C(C)(C)[C@H]1Oc1cnc(C#N)c(Cl)c1] | CB26 | 59.5 mg, 71% yield |
| | N-((1r,3r)-3-((5-Chloro-6-cyanopyridin-3-yl)oxy)-2,2,4,4- | | |
| | tetramethylcyclobutyl)-6-(4-((4-(4-(2,4- | | |
| | dioxotetrahydropyrimidin-1(2H)-yl)-1H-pyrrolo[2,3-b]pyridin- | | |
| | 1-yl)piperidin-1-yl)methyl)piperidin-1-yl)nicotinamide | | |
| | LCMS (formic A): Rt = 0.93 min, MH⁺ = 793 | | |
| EAR256 | 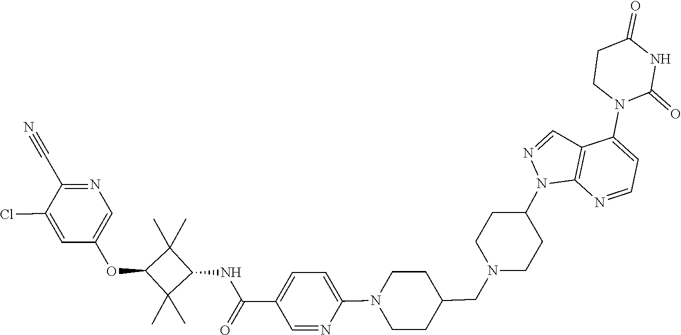 [SMILES: CC1(C)[C@H](NC(=O)c2ccc(N3CCC(CN4CCC(n5ncc6c(N7CCC(=O)NC7=O)ccnc65)CC4)CC3)nc2)C(C)(C)[C@H]1Oc1cnc(C#N)c(Cl)c1] | CB19 | 6.8 mg, 5% yield |
| | N-((1r,3r)-3-((5-Chloro-6-cyanopyridin-3-yl)oxy)-2,2,4,4- | | |
| | tetramethylcyclobutyl)-6-(4-((4-(4-(2,4- | | |
| | dioxotetrahydropyrimidin-1(2H)-yl)-1H-pyrazolo[3,4- | | |
| | b]pyridin-1-yl)piperidin-1-yl)methyl)piperidin-1- | | |
| | yl)nicotinamide | | |
| | LCMS (formic A): Rt = 0.93 min, MH⁺ = 794. | | |
| EAR257 | 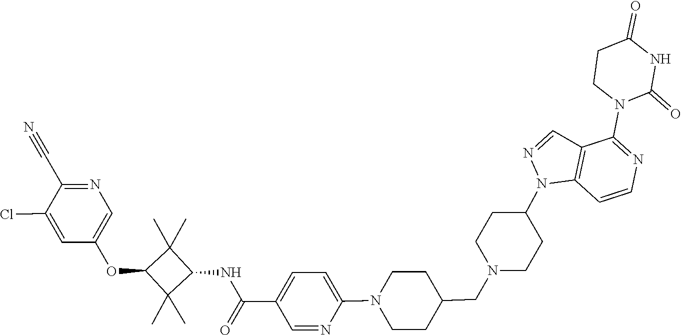 [SMILES: CC1(C)[C@H](NC(=O)c2ccc(N3CCC(CN4CCC(n5ncc6c(N7CCC(=O)NC7=O)nccc65)CC4)CC3)nc2)C(C)(C)[C@H]1Oc1cnc(C#N)c(Cl)c1] | CB7 | 109 mg, 81% yield |
| | N-((1r,3r)-3-((5-Chloro-6-cyanopyridin-3-yl)oxy)-2,2,4,4- | | |
| | tetramethylcyclobutyl)-6-(4-((4-(4-(2,4- | | |
| | dioxotetrahydropyrimidin-1(2H)-yl)-1H-pyrazolo[4,3- | | |
| | c]pyridin-1-yl)piperidin-1-yl)methyl)piperidin-1- | | |
| | yl)nicotinamide | | |
| | LCMS (formic A): Rt = 0.92 min, MH⁺ = 794 | | |
| EAR258 | 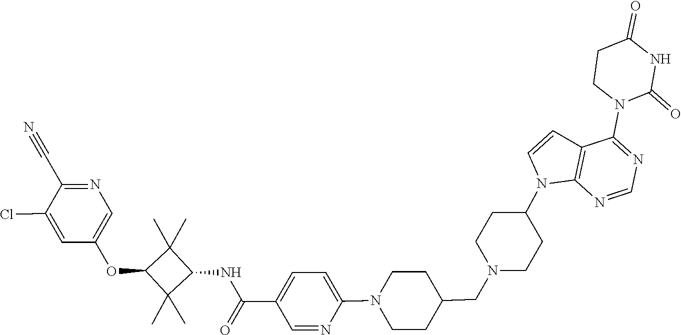 [SMILES: CC1(C)[C@H](NC(=O)c2ccc(N3CCC(CN4CCC(n5ccc6c(N7CCC(=O)NC7=O)ncnc65)CC4)CC3)nc2)C(C)(C)[C@H]1Oc1cnc(C#N)c(Cl)c1] | CB17 | 37 mg, 26% yield |
| | N-((1r,3r)-3-((5-Chloro-6-cyanopyridin-3-yl)oxy)-2,2,4,4- | | |
| | tetramethylcyclobutyl)-6-(4-((4-(4-(2,4- | | |
| | dioxotetrahydropyrimidin-1(2H)-yl)-7H-pyrrolo[2,3- | | |
| | d]pyrimidin-7-yl)piperidin-1-yl)methyl)piperidin-1- | | |
| | yl)nicotinamide | | |
| | LCMS (formic A): Rt = 0.93 min, MH⁺ = 794. | | |
| EAR259 | 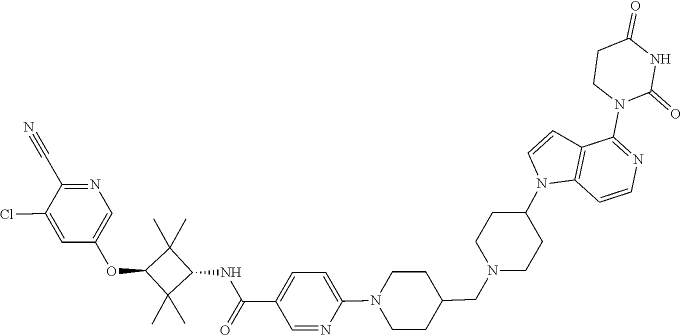 [SMILES: CC1(C)[C@H](NC(=O)c2ccc(N3CCC(CN4CCC(n5ccc6c(N7CCC(=O)NC7=O)nccc65)CC4)CC3)nc2)C(C)(C)[C@H]1Oc1cnc(C#N)c(Cl)c1] | CB25 | 79 mg, 59% yield |
| | N-((1r,3r)-3-((5-Chloro-6-cyanopyridin-3-yl)oxy)-2,2,4,4- | | |
| | tetramethylcyclobutyl)-6-(4-((4-(4-(2,4- | | |
| | dioxotetrahydropyrimidin-1(2H)-yl)-1H-pyrrolo[3,2-c]pyridin- | | |
| | 1-yl)piperidin-1-yl)methyl)piperidin-1-yl)nicotinamide | | |
| | LCMS (formic A): Rt = 0.88 min, MH⁺ = 793 | | |

### Example AR260

### N-((1r,3r)-3-((8-Cyanoquinolin-5-yl)oxy)-2,2,4,4-tetramethylcyclobutyl)-5-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)picolinamide hydrochloride (EAR260)

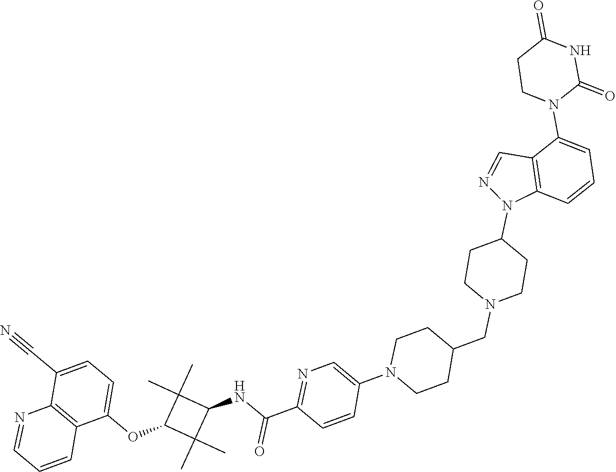 [SMILES: CC1(C)[C@H](NC(=O)c2ccc(N3CCC(CN4CCC(n5ncc6c(N7CCC(=O)NC7=O)cccc65)CC4)CC3)cn2)C(C)(C)[C@H]1Oc1ccc(C#N)c2ncccc12]

To a stirred solution of N-((1r,3r)-3-((8-cyanoquinolin-5-yl)oxy)-2,2,4,4-tetramethylcyclobutyl)-5-(4-(hydroxymethyl)piperidin-1-yl)picolinamide (may be prepared as described in Description 365; 104 mg, 202 μmol) in DCM (5 mL) was added DMP (111 mg, 263 μmol), then the mixture was stirred at room temperature for 3 h. The mixture was treated with Na₂S₂O₃ (10% aqueous, 5 mL), then the solution was extracted with DCM:methanol (9:1, 2×25 mL). The organic layers were combined, washed with brine (10 mL), dried over Na₂SO₄, filtered and concentrated under reduced pressure. The sample was purified by chromatography on Si (10 g) eluting with 95:5 DCM:methanol. The appropriate fractions were combined and evaporated in vacuo, then the sample was dissolved in DCM (4 mL), 1-(piperidin-4-yl)-1H-pyrazolo[3,4-b]pyridin-4-yl)dihydropyrimidine-2,4(1H,3H)-dione (may be prepared as described in Example ECB19, 48.7 mg, 0.155 mmol) and sodium triacetoxyborohydride (85.7 mg, 404 μmol) and the resulting mixture was stirred at room temperature for 14 h. The mixture was diluted with water (5 mL), and extracted with DCM:methanol (9:1, 2×25 mL). The organic layers were combined, washed with brine (10 mL), dried over Na₂SO₄, filtered and concentrated under reduced pressure. The sample was purified by preparative HPLC (XBridge 30 mm×100 mm) using a 40-100% acetonitrile-water (modified with 0.1% ammonium bicarbonate and adjusted to pH 10 with aqueous ammonia) gradient over 15 min (flow rate 40 mL/min). The appropriate fractions were combined and evaporated in vacuo, then the sample was was dissolved in DCM:methanol (3:1, 1 mL), treated with HCl in isopropanol (0.6M, 100 μL, 0.06 mmol) and stirred for 1 h. The solution was concentrated in vacuo to give the desired product (46.2 mg, 26% yield) as a white solid. LCMS (formic A): Rt=0.93 min, MH⁺=810.

### Example AR261

### N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-indazol-1-yl)piperidin-1-yl)methyl)piperidin-1-yl)nicotinamide hydrochloride (EAR261)

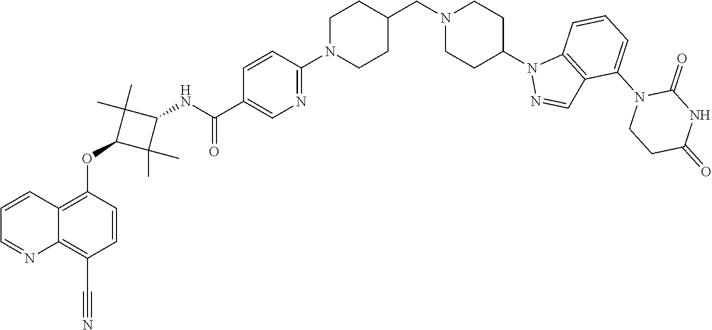 [SMILES: CC1(C)[C@H](NC(=O)c2ccc(N3CCC(CN4CCC(n5ncc6c(N7CCC(=O)NC7=O)cccc65)CC4)CC3)nc2)C(C)(C)[C@H]1Oc1ccc(C#N)c2ncccc12]

To a stirred solution of N-((1r,3r)-3-((8-cyanoquinolin-5-yl)oxy)-2,2,4,4-tetramethylcyclobutyl)-6-(4-(hydroxymethyl)piperidin-1-yl)nicotinamide (may be prepared as described in Description 355; 100 mg, 195 μmol) in DCM (2 mL) was added DMP (99.1 mg, 234 μmol) then the mixture was stirred at room temperature for 3 h. The mixture was diluted with a 1:1 mixture of NaHCO₃ (saturated aqueous) and Na₂CO₃ (10% aqueous) and DCM, then the phases were separated. The aqueous layer was back-extracted with DCM twice, then the organic layers were combined dried over anhydrous Na₂SO₄ and concentrated in vacuo. The sample was dissolved in DCE (3 mL), then 1-(1-(piperidin-4-yl)-1H-indazol-4-yl)dihydropyrimidine-2,4(1H,3H)-dione, trifluoroacetic acid salt (may be prepared as described in Example CB4, 83.5 mg, 195 μmol) and TEA (81.6 μL, 586 μmol) were added followed by sodium triacetoxyborohydride (57.9 mg, 273 μmol) then the mixture was stirred at room temperature for 16 h. The mixture was diluted with NaHCO₃ (saturated aqueous) and ethyl acetate then the phases were separated. The aqueous layer was back-extracted with ethyl acetate twice, then the organic layers were combined, dried over anhydrous sodium sulfate and concentrated in vacuo. The residue was triturated with ethanol, then the resulting solid was separated by filtration, and washed with additional ethanol. The solid was suspended in DCM (2 mL) and treated with HCl (0.6 M in isopropanol, 234 μmol) and stirred for 4 h. The sample was concentrated in vacuo to give the desired product (78 mg, 46% yield) as a white solid. LCMS (formic A): Rt=0.89 min, MH⁺=809.

### Example AR262

### N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyridazine-3-carboxamide (EAR262)

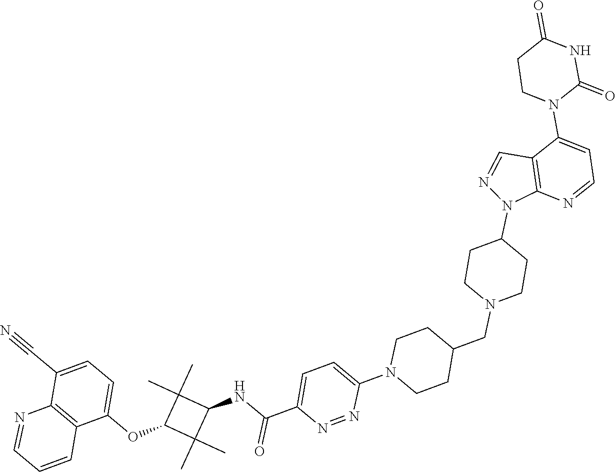 [SMILES: CC1(C)[C@H](NC(=O)c2ccc(N3CCC(CN4CCC(n5ncc6c(N7CCC(=O)NC7=O)ccnc65)CC4)CC3)nn2)C(C)(C)[C@H]1Oc1ccc(C#N)c2ncccc12]

To a suspension of N-((1r,3r)-3-((8-cyanoquinolin-5-yl)oxy)-2,2,4,4-tetramethylcyclobutyl)-6-(4-formylpiperidin-1-yl)pyridazine-3-carboxamide (may be prepared as described in Description 345, 75.0 mg, 146 μmol) and 1-(1-(piperidin-4-yl)-1H-pyrrolo[2,3-b]pyridin-4-yl)dihydropyrimidine-2,4(1H,3H)-dione (may be prepared as described in Example CB26, 45.8 mg, 146 μmol) in DCM (4 mL) and methanol (1 mL) was added sodium triacetoxyborohydride (93.0 mg, 439 μmol) and the mixture was stirred at room temperature for 18 h. The mixture was diluted with DCM and it was washed sequentially with water and brine. The organic layer was dried over Na₂SO₄, filtered and concentrated in vacuo. The sample was purified by preparative HPLC (XBridge 30 mm×100 mm) using a 40-100% acetonitrile-water (modified with 0.05% ammonium bicarbonate and aqueous ammonia to pH 10) gradient over 15 min (flow rate 40 mL/min). The appropriate fractions were combined and evaporated in vacuo to give the desired product (79.3 mg, 64% yield) as a white solid. LCMS (formic A): Rt=0.91 min, MH⁺=810.

### Example AR263

### N-((1r,3r)-3-((5-Chloro-6-cyanopyridin-3-yl)oxy)-2,2,4,4-tetramethylcyclobutyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyridazine-3-carboxamide (EAR263)

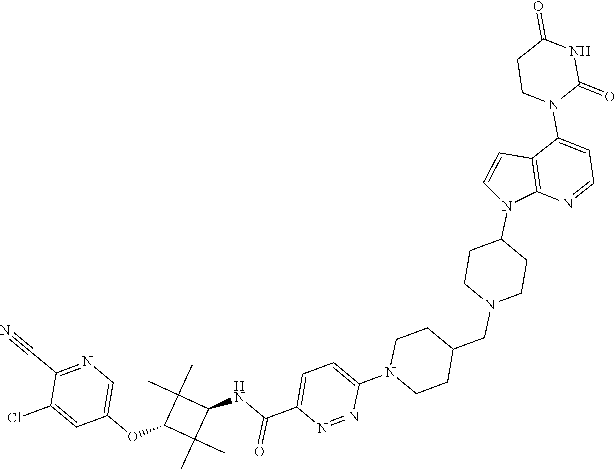 [SMILES: CC1(C)[C@H](NC(=O)c2ccc(N3CCC(CN4CCC(n5ccc6c(N7CCC(=O)NC7=O)ccnc65)CC4)CC3)nn2)C(C)(C)[C@H]1Oc1cnc(C#N)c(Cl)c1]

To a suspension of N-((1r,3r)-3-((5-chloro-6-cyanopyridin-3-yl)oxy)-2,2,4,4-tetramethylcyclobutyl)-6-(4-formylpiperidin-1-yl)pyridazine-3-carboxamide (prepared as an intermediate (not isolated) in Example EAR248; 125 mg, 252 μmol) and 1-(1-(piperidin-4-yl)-1H-pyrrolo[2,3-b]pyridin-4-yl)dihydropyrimidine-2,4(1H,3H)-dione (may be prepared as described in Example CB26, 87.6 mg, 252 μmol) in DCM (4 mL) and methanol (1 mL) was added sodium triacetoxyborohydride (160 mg, 755 μmol) and the mixture was stirred at room temperature for 18 h. The mixture was diluted with DCM and it was washed sequentially with water and brine. The organic layer was dried over Na₂SO₄, filtered and concentrated in vacuo. The sample was purified by preparative HPLC (XBridge 30 mm×100 mm) using a 40-100% acetonitrile-water (modified with 0.05% ammonium bicarbonate and aqueous ammonia to pH 10) gradient over 15 min (flow rate 40 ml/min). The appropriate fractions were combined and evaporated in vacuo to give the desired product (77 mg, 37% yield) as a white solid. LCMS (formic A): Rt=0.97 min, MH⁺=794.

### Example AR264

### N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[3,2-c]pyridin-1-yl)piperidin-1-yl)methyl)piperidin-1-yl)nicotinamide hydrochloride (EAR264)

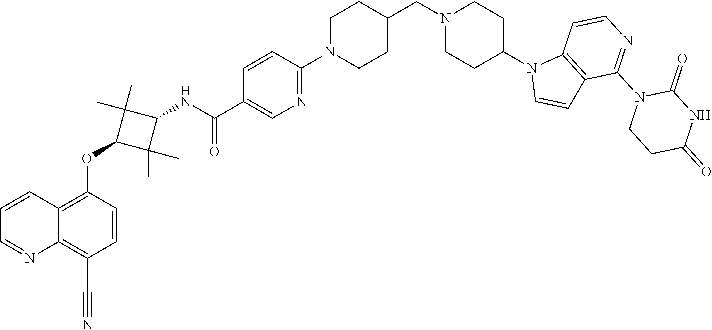 [SMILES: CC1(C)[C@H](NC(=O)c2ccc(N3CCC(CN4CCC(n5ccc6c(N7CCC(=O)NC7=O)nccc65)CC4)CC3)nc2)C(C)(C)[C@H]1Oc1ccc(C#N)c2ncccc12]

Sodium triacetoxyborohydride (58.7 mg, 277 μmol) was added to a stirred solution of N-((1r,3r)-3-((8-cyanoquinolin-5-yl)oxy)-2,2,4,4-tetramethylcyclobutyl)-6-(4-formylpiperidin-1-yl)nicotinamide (may be prepared as described in Description 362, 101 mg, 198 μmol), and 1-(1-(piperidin-4-yl)-1H-pyrrolo[3,2-c]pyridin-4-yl)dihydropyrimidine-2,4(1H,3H)-dione (may be prepared as described in Example ECB25, 62.0 mg, 198 μmol) in DCE (2 mL) and DMA (0.5 mL) then the mixture was stirred at room temperature for 16 h. The mixture was diluted with ethyl acetate and NaHCO₃ (saturated aqueous) then the phases were separated. The aqueous layer was extracted twice with EtOAc, then the organic layers were combined, dried over anhydrous sodium sulfate, filtered and evaporated in vacuo. The resulting sample was washed with water to give a white solid. The solid was suspended in DCM (2 mL) and treated with HCl (0.5M in MeOH, 435 μL, 218 μmol) then the mixture was stirred at room temperature for 4 h. The mixture was concentrated in vacuo to give the desired product (76.6 mg, 44% yield) as a white solid. LCMS (formic A): Rt=0.82 min, MH⁺=809.

### Example AR265

### N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)nicotinamide hydrochloride (EAR265)

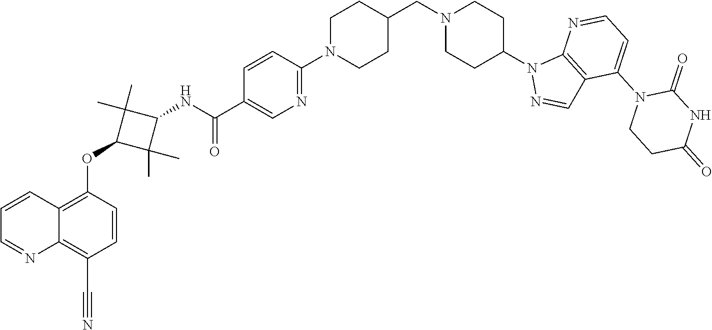 [SMILES: CC1(C)[C@H](NC(=O)c2ccc(N3CCC(CN4CCC(n5ncc6c(N7CCC(=O)NC7=O)ccnc65)CC4)CC3)nc2)C(C)(C)[C@H]1Oc1ccc(C#N)c2ncccc12]

Sodium triacetoxyborohydride (49.0 mg, 231 μmol) was added to a stirred solution of N-((1r,3r)-3-((8-cyanoquinolin-5-yl)oxy)-2,2,4,4-tetramethylcyclobutyl)-6-(4-formylpiperidin-1-yl)nicotinamide (may be prepared as described in Description 362, 84.5 mg, 165 μmol), and 1-(1-(piperidin-4-yl)-1H-pyrazolo[3,4-b]pyridin-4-yl)dihydropyrimidine-2,4(1H,3H)-dione (may be prepared as described in Example ECB19, 51.9 mg, 165 μmol) in DCE (2 mL) and DMA (0.5 mL) then the mixture was stirred at room temperature for 16 h. The mixture was diluted with ethyl acetate and NaHCO₃ (saturated aqueous) then the phases were separated. The aqueous layer was extracted twice with ethyl acetate, then the organic layers were combined, dried over anhydrous sodium sulfate, filtered and evaporated in vacuo. The residue was triturated sequentially with diethyl ether and ethanol to give a white solid. The solid was suspended in DCM (2 mL) and treated with HCl (0.5M in isopropanol, 363 182 μmol) then stirred for 4 h. The mixture was concentrated in vacuo to give the desired product (103 mg, 73% yield) as a white solid. LCMS (formic A): Rt=0.87 min, MH⁺=810.

### Example AR266

### N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)nicotinamide hydrochloride (EAR266)

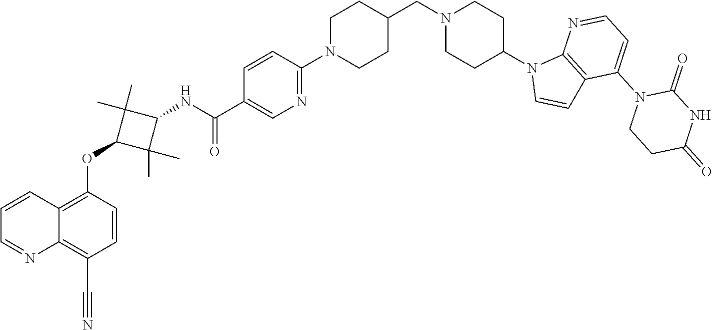 [SMILES: CC1(C)[C@H](NC(=O)c2ccc(N3CCC(CN4CCC(n5ccc6c(N7CCC(=O)NC7=O)ccnc65)CC4)CC3)nc2)C(C)(C)[C@H]1Oc1ccc(C#N)c2ncccc12]

Sodium triacetoxyborohydride (56.2 mg, 265 μmol) was added to a stirred solution of N-((1r,3r)-3-((8-cyanoquinolin-5-yl)oxy)-2,2,4,4-tetramethylcyclobutyl)-6-(4-formylpiperidin-1-yl)nicotinamide (may be prepared as described in Description 362, 97.0 mg, 190 μmol), 1-(1-(piperidin-4-yl)-1H-pyrrolo[2,3-b]pyridin-4-yl)dihydropyrimidine-2,4(1H,3H)-dione trifluoroacetic acid salt (may be prepared as described in Example ECB26, 90.0 mg, 90% w/w, 190 μmol) and TEA (79.2 μL, 569 μmol) in DCE (2 mL) and DMA (0.5 mL) then the mixture was stirred at room temperature for 16 h. The mixture was diluted with ethyl acetate and NaHCO₃ (saturated aqueous) then the phases were separated. The aqueous layer was extracted twice with ethyl acetate, then the organic layers were combined, dried over anhydrous sodium sulfate, filtered and evaporated in vacuo. The residue was triturated with ethanol, then the resulting solid was separated by filtration and collected. The filtrate was separately concentrated in vacuo and combined with the triturated solid. The combined sample was triturated with water to give a white solid, which was suspended in DCM (2 mL) and treated with HCl (0.5 M in methanol, 417 μL, 208 μmol) then the mixture was stirred for 4 h. The mixture was concentrated in vacuo to give the desired product (114 mg, 69% yield) as a white solid. LCMS (formic A): Rt=0.87 min, MH⁺=809.

### Example AR267

### N-((1r,3r)-3-((8-Cyanoquinolin-5-yl)oxy)-2,2,4,4-tetramethylcyclobutyl)-6-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)nicotinamide hydrochloride (EAR267)

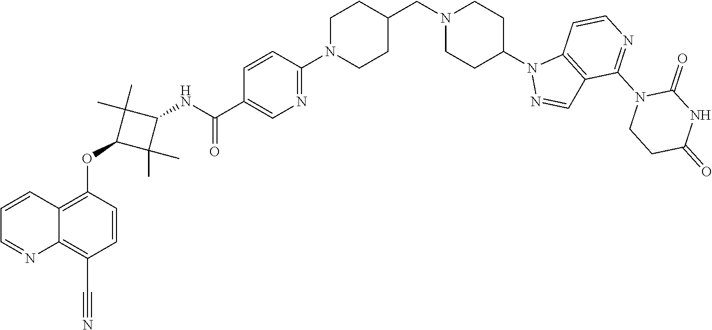 [SMILES: CC1(C)[C@H](NC(=O)c2ccc(N3CCC(CN4CCC(n5ncc6c(N7CCC(=O)NC7=O)nccc65)CC4)CC3)nc2)C(C)(C)[C@H]1Oc1ccc(C#N)c2ncccc12]

Sodium triacetoxyborohydride (51.9 mg, 245 μmol) was added to a stirred solution of N-((1r,3r)-3-((8-cyanoquinolin-5-yl)oxy)-2,2,4,4-tetramethylcyclobutyl)-6-(4-formylpiperidin-1-yl)nicotinamide (may be prepared as described in Description 362, 89.5 mg, 175 μmol), and 1-(1-(piperidin-4-yl)-1H-pyrazolo[4,3-c]pyridin-4-yl)dihydropyrimidine-2,4(1H,3H)-dione (may be prepared as described in Example ECB7, 55.0 mg, 175 μmol) in DCE (2.0 mL) and DMA (0.5 mL), then the mixture was stirred at room temperature for 16 h. The mixture was diluted with ethyl acetate and NaHCO₃ (saturated aqueous) then the phases were separated. The aqueous layer was extracted twice with ethyl acetate, then the organic layers were combined, dried over anhydrous sodium sulfate, filtered and evaporated in vacuo. The residue was triturated sequentially with diethyl ether and ethanol to give a white solid. The solid was suspended in DCM (2 mL) and treated with HCl (0.5M in isopropanol, 385 μL, 192 μmol) then the mixture stirred for 4 h. The mixture was concentrated in vacuo to give the desired product (104 mg, 70% yield) as a white solid. LCMS (formic A): Rt=0.86 min, MH⁺=810.

### Example AR268

### N-((1r,3r)-3-((5-Chloro-6-cyanopyridin-3-yl)oxy)-2,2,4,4-tetramethylcyclobutyl)-5-(4-((4-(4-(2,4-dioxotetrahydropyrimidin-1(2H)-yl)-1H-indazol-1-yl)piperidin-1-yl)methyl)piperidin-1-yl)pyrimidine-2-carboxamide (EAR268)

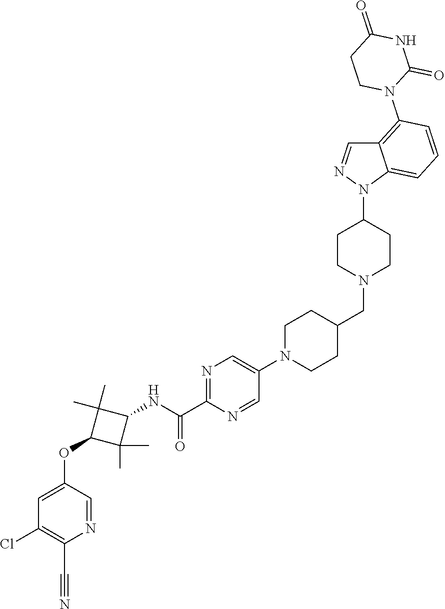 [SMILES: CC1(C)[C@H](NC(=O)c2ncc(N3CCC(CN4CCC(n5ncc6c(N7CCC(=O)NC7=O)cccc65)CC4)CC3)cn2)C(C)(C)[C@H]1Oc1cnc(C#N)c(Cl)c1]

To a suspension of N-((1r,3r)-3-((5-chloro-6-cyanopyridin-3-yl)oxy)-2,2,4,4-tetramethylcyclobutyl)-5-(4-formylpiperidin-1-yl)pyrimidine-2-carboxamide (may be prepared as described in Description 369; 74.5 mg, 150 μmol) and 1-(1-(piperidin-4-yl)-1H-indazol-4-yl)dihydropyrimidine-2,4(1H,3H)-dione (may be prepared as described in Example ECB4, 47 mg, 150 μmol) in DCE (2.4 mL) and DMA (0.6 mL) was added sodium triacetoxyborohydride (95.4 mg, 450 μmol), then the resulting mixture was stirred at room temperature for 18 h. The mixture was diluted with DCM and water, then the phases were separated. The aqueous layer was back-extracted with DCM twice, then the organic layers were combined, dried over Na₂SO₄, filtered and the solvent removed under reduced pressure. The sample was purified by preparative HPLC (XBridge 30 mm×100 mm) using a 40-100% acetonitrile-water (+0.05% ammonium bicarbonate modifier) gradient over 15 min (flow rate 40 mL/min). The appropriate fractions were combined and evaporated in vacuo to give the desired product (77 mg, 61% yield) as a white solid. LCMS (formic A): Rt=0.94 min, MH⁺=794.

### Example AR269

### N-((1r,3r)-3-((5-Chloro-6-cyanopyridin-3-yl)oxy)-2,2,4,4-tetramethylcyclobutyl)-5-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyrimidine-2-carboxamide (EAR269)

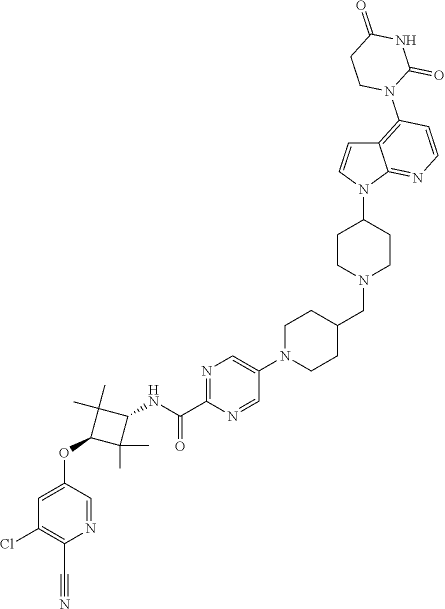 [SMILES: CC1(C)[C@H](NC(=O)c2ncc(N3CCC(CN4CCC(n5ccc6c(N7CCC(=O)NC7=O)ccnc65)CC4)CC3)cn2)C(C)(C)[C@H]1Oc1cnc(C#N)c(Cl)c1]

To a suspension of N-((1r,3r)-3-((5-chloro-6-cyanopyridin-3-yl)oxy)-2,2,4,4-tetramethylcyclobutyl)-5-(4-formylpiperidin-1-yl)pyrimidine-2-carboxamide (may be prepared as described in Description 369; 74.5 mg, 150 μmol) and 1-(1-(piperidin-4-yl)-1H-pyrrolo[2,3-b]pyridin-4-yl)dihydropyrimidine-2,4(1H,3H)-dione (may be prepared as described in Example ECB26, 47 mg, 150 μmol) in DCE (2.4 mL) and DMA (0.6 mL) was added sodium triacetoxyborohydride (95.4 mg, 450 μmol), then the resulting mixture was stirred at room temperature for 18 h. The mixture was diluted with DCM and water, then the phases were separated. The aqueous layer was back-extracted with DCM twice, then the organic layers were combined, dried over Na₂SO₄, filtered and the solvent removed under reduced pressure. The sample was purified by preparative HPLC (XBridge 30 mm×100 mm) using a 40-100% acetonitrile-water (+0.05% ammonium bicarbonate modifier) gradient over 15 min (flow rate 40 mL/min), then the appropriate fractions were combined and evaporated in vacuo to give the desired product (65.3 mg, 52% yield) as a white solid. LCMS (formic A): Rt=0.94 min, MH⁺=794.

### Example AR270

### N-((1r,3r)-3-((6-Cyano-5-(trifluoromethyl)pyridin-3-yl)oxy)-2,2,4,4-tetramethylcyclobutyl)-5-(4-((4-(4-(2,4-dioxotetrahydropyrimidin-1(2H)-yl)-1H-indazol-1-yl)piperidin-1-yl)methyl)piperidin-1-yl)pyrimidine-2-carboxamide (EAR270)

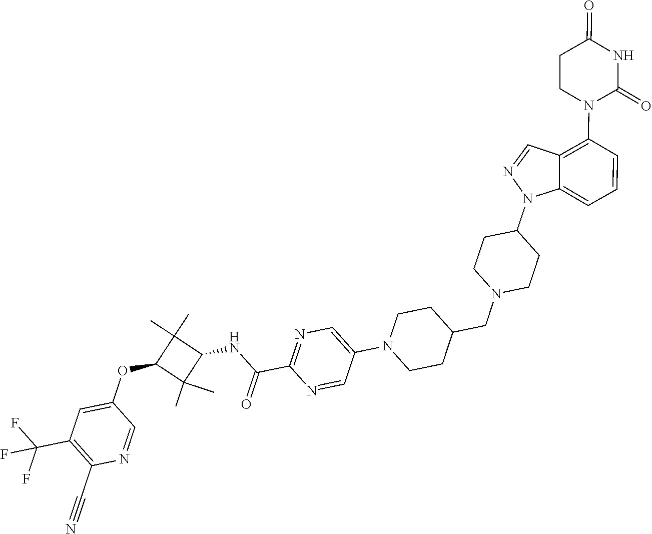 [SMILES: CC1(C)[C@H](NC(=O)c2ncc(N3CCC(CN4CCC(n5ncc6c(N7CCC(=O)NC7=O)cccc65)CC4)CC3)cn2)C(C)(C)[C@H]1Oc1cnc(C#N)c(C(F)(F)F)c1]

To a suspension of N-((1r,3r)-3-((6-cyano-5-(trifluoromethyl)pyridin-3-yl)oxy)-2,2,4,4-tetramethylcyclobutyl)-5-(4-formylpiperidin-1-yl)pyrimidine-2-carboxamide (may be prepared as described in Description 373; 102 mg, 191 μmol) and 1-(1-(piperidin-4-yl)-1H-indazol-4-yl)dihydropyrimidine-2,4(1H,3H)-dione (may be prepared as described in Example ECB4, 60 mg, 191 μmol) in DCE (2.5 mL) and DMA (0.625 mL) was added sodium triacetoxyborohydride (122 mg, 574 μmol), then the resulting mixture was stirred at room temperature for 18 h. The mixture was diluted with DCM and water, then the phases were separated. The aqueous layer was back-extracted with DCM twice, then the organic layers were combined, dried over Na₂SO₄, filtered and the solvent removed under reduced pressure. The sample was purified by preparative HPLC (XBridge 30 mm×100 mm) using a 40-100% acetonitrile-water (+0.05% ammonium bicarbonate modifier) gradient over 15 min (flow rate 40 mL/min), then the appropriate fractions were combined and evaporated in vacuo to give the desired product (85 mg, 51% yield) as a white solid. LCMS (formic A): Rt=0.98 min, MH+=828.

### Example AR271

### N-((1r,3r)-3-((6-Cyano-5-(trifluoromethyl)pyridin-3-yl)oxy)-2,2,4,4-tetramethylcyclobutyl)-5-(4-((4-(4-(2,4-dioxotetrahydropyrimidin-1(2H)-yl)-1H-pyrrolo[2,3-b]pyridin-1-yl)piperidin-1-yl)methyl)piperidin-1-yl)pyrimidine-2-carboxamide (EAR271)

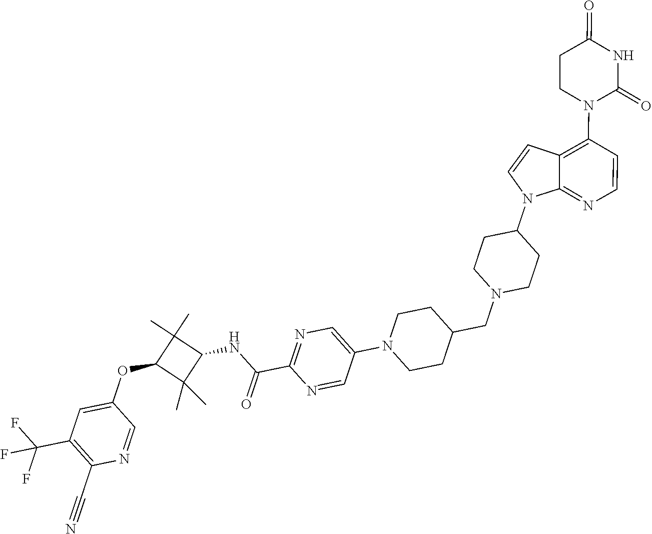 [SMILES: CC1(C)[C@H](NC(=O)c2ncc(N3CCC(CN4CCC(n5ccc6c(N7CCC(=O)NC7=O)ccnc65)CC4)CC3)cn2)C(C)(C)[C@H]1Oc1cnc(C#N)c(C(F)(F)F)c1]

To a suspension of N-((1r,3r)-3-((6-cyano-5-(trifluoromethyl)pyridin-3-yl)oxy)-2,2,4,4-tetramethylcyclobutyl)-5-(4-formylpiperidin-1-yl)pyrimidine-2-carboxamide (may be prepared as described in Description 373; 102 mg, 191 μmol) and 1-(1-(piperidin-4-yl)-1H-pyrrolo[2,3-b]pyridin-4-yl)dihydropyrimidine-2,4(1H,3H)-dione (may be prepared as described in Example ECB26, 60 mg, 191 μmol) in DCE (3 mL) and DMA (0.75 mL) was added sodium triacetoxyborohydride (122 mg, 574 μmol), then the resulting mixture was stirred at room temperature for 18 h. The mixture was diluted with DCM and water, then the phases were separated. The aqueous layer was back-extracted with DCM twice, then the organic layers were combined, dried over Na₂SO₄, filtered and the solvent removed under reduced pressure. The sample was purified by preparative HPLC (XBridge 30 mm×100 mm) using a 40-100% acetonitrile-water (+0.1% ammonium bicarbonate modifier adjusted to pH 10 with aqueous ammonia) gradient over 15 min (flow rate 40 ml/min), then the appropriate fractions were combined and evaporated in vacuo to give the desired product (106 mg, 64% yield) as a white solid. LCMS (formic A): Rt=0.97 min, MH+=828.

### Example AR272

### N-((1r,3r)-3-(3-Chloro-4-cyanophenoxy)-2,2,4,4-tetramethylcyclobutyl)-5-(4-((4-(4-(2,4-dioxotetrahydropyrimidin-1(2H)-yl)-1H-pyrazolo[3,4-b]pyridin-1-yl)piperidin-1-yl)methyl)piperidin-1-yl)pyrimidine-2-carboxamide (EAR272)

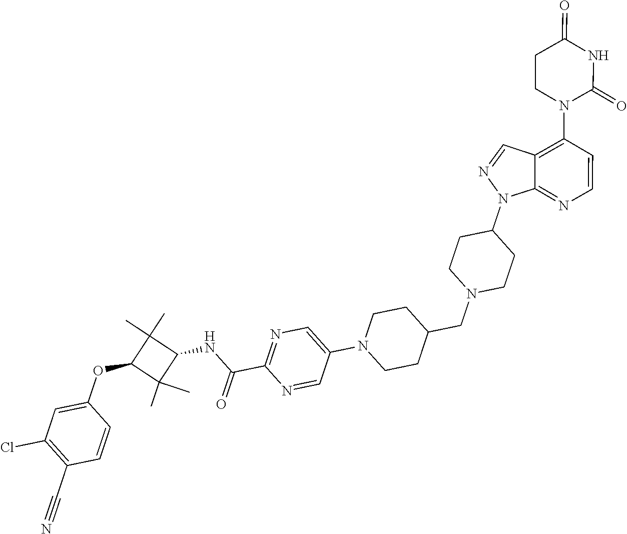 [SMILES: CC1(C)[C@H](NC(=O)c2ncc(N3CCC(CN4CCC(n5ncc6c(N7CCC(=O)NC7=O)ccnc65)CC4)CC3)cn2)C(C)(C)[C@H]1Oc1ccc(C#N)c(Cl)c1]

To a suspension of N-((1r,3r)-3-(3-chloro-4-cyanophenoxy)-2,2,4,4-tetramethylcyclobutyl)-5-(4-formylpiperidin-1-yl)pyrimidine-2-carboxamide (may be prepared as described in Description 375; 63.1 mg, 127 μmol) and 1-(1-(piperidin-4-yl)-1H-pyrazolo[3,4-b]pyridin-4-yl)dihydropyrimidine-2,4(1H,3H)-dione (may be prepared as described in Example ECB19; 40 mg, 127 μmol) in DCE (2 mL) and DMA (0.5 mL) was added sodium triacetoxyborohydride (80.9 mg, 381 μmol), then the resulting mixture was stirred at room temperature for 18 h. The mixture was diluted with DCM and water, then the phases were separated. The aqueous layer was back-extracted with DCM twice, then the organic layers were combined, dried over Na₂SO₄, filtered and the solvent removed under reduced pressure. The sample was purified by preparative HPLC (XBridge 30 mm×100 mm) using a 40-100% acetonitrile-water (+0.05% ammonium bicarbonate modifier) gradient over 15 min (flow rate 40 mL/min), then the appropriate fractions were combined and evaporated in vacuo to give the desired product (54.1 mg, 51% yield) as a white solid. LCMS (formic A): Rt=0.98 min, MH⁺=794.

### Example AR273

### N-((1r,3r)-3-(3-Chloro-4-cyanophenoxy)-2,2,4,4-tetramethylcyclobutyl)-5-(4-((4-(4-(2,4-dioxotetrahydropyrimidin-1(2H)-yl)-1H-pyrazolo[4,3-c]pyridin-1-yl)piperidin-1-yl)methyl)piperidin-1-yl)pyrimidine-2-carboxamide (EAR273)

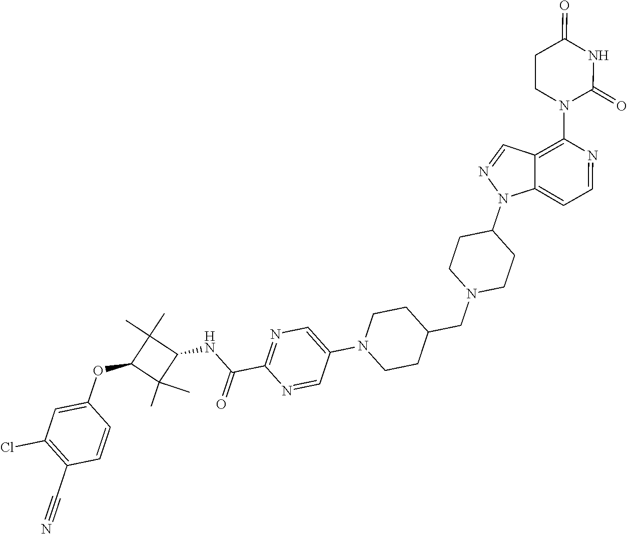 [SMILES: CC1(C)[C@H](NC(=O)c2ncc(N3CCC(CN4CCC(n5ncc6c(N7CCC(=O)NC7=O)nccc65)CC4)CC3)cn2)C(C)(C)[C@H]1Oc1ccc(C#N)c(Cl)c1]

To a suspension of N-((1r,3r)-3-(3-chloro-4-cyanophenoxy)-2,2,4,4-tetramethylcyclobutyl)-5-(4-formylpiperidin-1-yl)pyrimidine-2-carboxamide (may be prepared as described in Description 375; 63.1 mg, 127 μmol) and 1-(1-(piperidin-4-yl)-1H-pyrazolo[4,3-c]pyridin-4-yl)dihydropyrimidine-2,4(1H,3H)-dione (may be prepared as described in Example ECB7; 40.0 mg, 127 μmol) in DCE (2 mL) and DMA (0.5 mL) was added sodium triacetoxyborobydride (80.9 mg, 382 μmol), then the resulting mixture was stirred at room temperature for 18 h. The mixture was diluted with DCM and water, then the phases were separated. The aqueous layer was back-extracted with DCM twice, then the organic layers were combined, dried over Na₂SO₄, filtered and the solvent removed under reduced pressure. The sample was purified by preparative HPLC (XBridge 30 mm×100 mm) using a 40-100% acetonitrile-water (+0.1% ammonium bicarbonate modifier modified to pH 10 with aqueous ammonia) gradient over 15 min (flow rate 40 mL/min), then the appropriate fractions were combined and evaporated in vacuo to give the desired product (56.8 mg, 53% yield) as a white solid. LCMS (formic A): Rt=0.97 min, MH⁺=794.

### RIPK2 PROTAC Examples

The following compounds are compounds of formula (I), (Ia) or (Ib) where p and q are both 1 and the TBM is a RIPK2 binding moiety.

### Example R1

### 1-(1-(1-(8-((4-(Benzo[d]thiazol-5-ylamino)-6-(tert-butylsulfonyl)quinolin-7-yl)oxy)octanoyl)piperidin-4-yl)-1H-indol-4-yl)dihydropyrimidine-2,4(1H,3H)-dione (ER1)

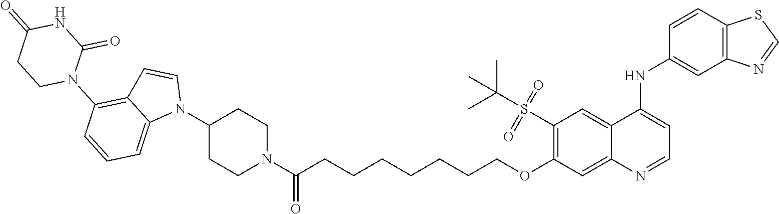 [SMILES: CC(C)(C)S(=O)(=O)c1cc2c(Nc3ccc4scnc4c3)ccnc2cc1OCCCCCCCC(=O)N1CCC(n2ccc3c(N4CCC(=O)NC4=O)cccc32)CC1]

A mixture of 8-((4-(benzo[d]thiazol-5-ylamino)-6-(tert-butylsulfonyl)quinolin-7-yl)oxy)octanoic acid (may be prepared as described in Description 175; 34 mg, 0.061 mmol) and HATU (46 mg, 0.121 mmol) in anhydrous DMF (0.6 mL) was treated with DIPEA (0.053 mL, 0.304 mmol) and the solution stirred at room temperature in a stoppered vessel for 20 min. 1-(1-(Piperidin-4-yl)-1H-indol-4-yl)dihydropyrimidine-2,4(1H,3H)-dione (may be prepared as described in Example CB1; 19 mg, 0.061 mmol) was added to the reaction mixture which was stirred at room temperature in a stoppered vessel for 64 h. The solution was diluted with DMSO (1.5 mL) and purified by MDAP (high pH). The appropriate fraction was evaporated in vacuo. The residue was dissolved in 1:1 acetonitrile:methanol (4 mL), transferred to a vial and the solvent evaporated under a stream of nitrogen. The solid was dried in a vacuum oven to give the title compound as a yellow solid (25 mg, 0.029 mmol, 48% yield). LCMS (high pH): Rt=1.15 min, MH+=850

### Example R2

### 1-(1-(1-(5-(1-(3-((4-(Benzo[d]thiazol-5-ylamino)-6-(tert-butylsulfonyl)quinolin-7-yl)oxy)propyl)piperidin-4-yl)pyrazine-2-carbonyl)piperidin-4-yl)-1H-indol-4-yl)dihydropyrimidine-2,4(1H,3H)-dione formate (ER2)

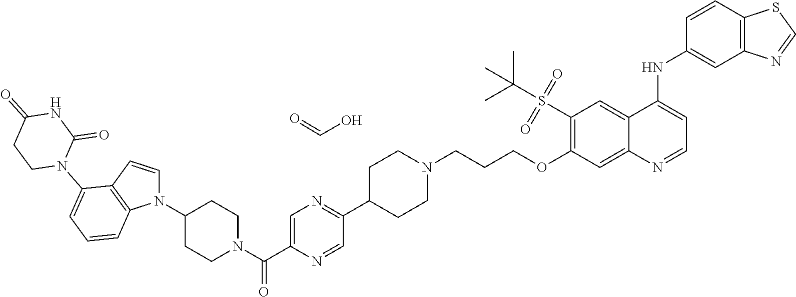 [SMILES: CC(C)(C)S(=O)(=O)c1cc2c(Nc3ccc4scnc4c3)ccnc2cc1OCCCN1CCC(c2cnc(C(=O)N3CCC(n4ccc5c(N6CCC(=O)NC6=O)cccc54)CC3)cn2)CC1 ; O=CO]

A mixture of 5-(1-(3-((4-(benzo[d]thiazol-5-ylamino)-6-(tert-butylsulfonyl)quinolin-7-yl)oxy)propyl)piperidin-4-yl)pyrazine-2-carboxylic acid dihydrochloride (may be prepared as described in Description 179; 47.0 mg, 0.064 mmol) and HATU (32 mg, 0.084 mmol) in anhydrous DMF (0.6 mL) was treated with DIPEA (0.045 mL, 0.256 mmol) and the solution left to stand at room temperature in a stoppered vessel for 20 min. 1-(1-(piperidin-4-yl)-1H-indol-4-yl)dihydropyrimidine-2,4(1H,3H)-dione (may be prepared as described in Example CB1; 20 mg, 0.064 mmol) was added to the reaction mixture which was left to stand at room temperature in a stoppered vessel for 2 h. Further HATU (16 mg) was added and the mixture left to stand at room temperature in a stoppered vessel for 22 h. The solution was diluted with DMSO (0.2 mL) and purified by MDAP (formic). The appropriate fractions were combined and the solvent evaporated under a nitrogen stream to give the title compound as a yellow solid (34 mg, 0.034 mmol, 53% yield). LCMS (formic A): Rt=0.56 min, MH+=378

### Example R3

### N-(2-(2-(2-((4-(Benzo[d]thiazol-5-ylamino)-6-(tert-butylsulfonyl)quinolin-7-yl)oxy)ethoxy)ethoxy)ethyl)-4-(2,4-dioxotetrahydropyrimidin-1(2H)-yl)-1-isopropyl-1H-indole-6-carboxamide (ER3)

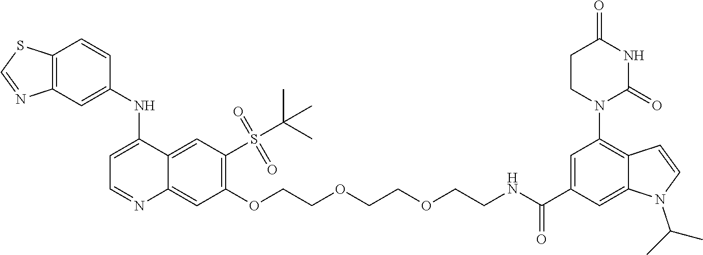 [SMILES: CC(C)n1ccc2c(N3CCC(=O)NC3=O)cc(C(=O)NCCOCCOCCOc3cc4nccc(Nc5ccc6scnc6c5)c4cc3S(=O)(=O)C(C)(C)C)cc21]

A solution of N-(7-(2-(2-(2-aminoethoxy)ethoxy)ethoxy)-6-(tert-butylsulfonyl)quinolin-4-yl)benzo[d]thiazol-5-amine (may be prepared as described in Description 181; 34.5 mg, 0.063 mmol), 4-(2,4-dioxotetrahydropyrimidin-1(2H)-yl)-1-isopropyl-1H-indole-6-carboxylic acid (may be prepared as described in Description 35; 20 mg, 0.063 mmol) and triethylamine (0.035 mL, 0.254 mmol) in DMF (0.8 mL) was treated with HATU (33.8 mg, 0.089 mmol) and stirred at ambient temperature for 30 min. The mixture was purified by MDAP (high pH) to afford the title compound (38 mg, 0.045 mmol, 71% yield). LCMS (high pH A): Rt=1.02 min, MH⁺=842

### IRAK4 PROTAC Examples

The following compounds are compounds of formula (I), (Ia) or (Ib) where p and q are both 1 and the TBM is a IRAK4 binding moiety.

### Example I1

### 2-(2-((Cyclopropylmethyl)amino)pyridin-4-yl)-N-(3-(difluoromethyl)-1-(4-((2-(2-(2-(4-(2,4-dioxotetrahydropyrimidin-1(2H)-yl)-1-isopropyl-1H-indole-6-carboxamido)ethoxy)ethoxy)ethyl)(methyl)carbamoyl)phenyl)-1H-pyrazol-4-yl)oxazole-4-carboxamide (EI1)

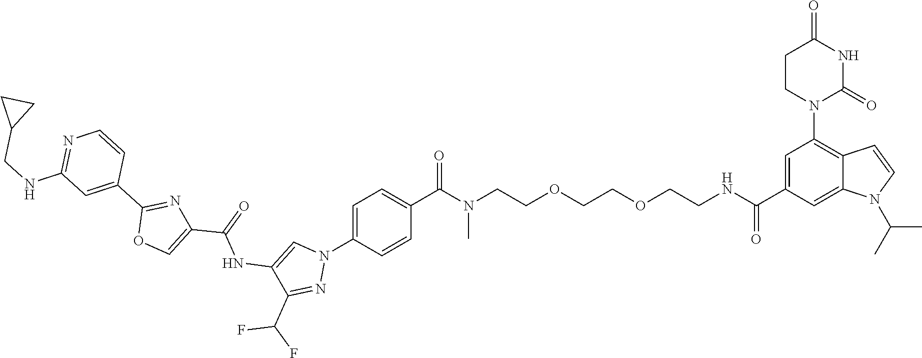 [SMILES: CC(C)n1ccc2c(N3CCC(=O)NC3=O)cc(C(=O)NCCOCCOCCN(C)C(=O)c3ccc(-n4cc(NC(=O)c5coc(-c6ccnc(NCC7CC7)c6)n5)c(C(F)F)n4)cc3)cc21]

tert-Butyl (cyclopropylmethyl) (4-(4-((3-(difluoromethyl)-1-(4-((2-(2-(2-(4-(2,4-dioxotetrahydropyrimidin-1(2H)-yl)-1-isopropyl-1H-indole-6-carboxamido)ethoxy)ethoxy)ethyl)(methyl)carbamoyl)phenyl)-1H-pyrazol-4-yl)carbamoyl)oxazol-2-yl)pyridin-2-yl)carbamate (may be prepared as described in Description 187; 20 mg, 0.019 mmol) and 4.0 M HCl in dioxane (241 μl, 0.965 mmol) were mixed and stirred for 4 h. The mixture was blown down and purified by MDAP (high pH) and freeze-dried overnight (approximately 20 h) to give the title compound (10 mg, 10.15 μmol, 53% yield). LCMS (high pH A): Rt=1.15 min, MH+=936

### BIOLOGICAL DATA

### Cereblon Binding Assay Protocol

Full length Cereblon protein (6His tev Cereblon/6His tev DDB1ΔBPB) was expressed and purified from E. coli to monitor compound binding. An AlexaFluor 647 labeled compound (lenalidomide labeled with Alexa647 fluorescent dye) was synthesised in house by solution synthesis using AlexaFluor 647 succinimidyl ester as labelling reagent. Compounds were titrated 1:3 from 10 mM in 100% DMSO and 100 nL transferred to a low volume black 384 well microtitre plate using a Labcyte Echo 555. A Thermo Scientific Multidrop Combi was used to dispense 5 μL of 10 nM protein in assay buffer of 50 mM

HEPES, 150 mM NaCl, 5% glycerol, 1 mM DTT and 1 mM CHAPS, pH 7.4, in the presence of 100 nM fluorescent ligand (Final assay concentrations of 5 nM protein and 50 nM ligand). After equilibrating for 15 min in the dark at room temperature a 5 μL addition was made of 2 nM europium chelate labelled anti-6His antibody (Perkin Elmer, W1024, AD0111) in assay buffer (1 nM FAC). Time resolved fluorescence (TRF) was then detected after a further 30 min incubation in the dark at room temperature on a TRF laser equipped Perkin Elmer Envision multimode plate reader (excitation=337 nm; emission 1=615 nm; emission 2=665 nm; dual wavelength bias dichroic=400 nm, 630 nm). TR-FRET ratio was calculated using the following equation: Ratio=((Acceptor fluorescence at 665 nm)/(Donor fluorescence at 615 nm)). TR-FRET ratio data was normalised to high (DMSO) and low (reaction in the absence of protein) controls and pIC₅₀ values determined for each of the compounds tested by fitting the fluorescence ratio data to a four parameter model:

$$y = A + \frac{B - A}{1 + {( \frac{{10}^{c}}{x} )}^{D}}$$

where ‘a’ is the minimum, ‘b’ is the maximum, ‘c’ is the pIC₅₀ and ‘d’ is the Hill slope.

### Results

ECB1, ECB6, ECB7, ECB8, ECB9, ECB10, ECB11, ECB15, ECB16, ECB17, ECB18, ECB19, ECB25, ECB29, ECB30, ECB37, ECB44, ECB45, ECB46, ECB47, ECB48, ECB49, ECB50, ECB51 were tested in the Cereblon Binding Assay. Each of these compounds had a mean pIC50≥6.0. Data for individual compounds is set out below:

| Example | Mean pIC50 |
|---|---|
| ECB1 | 8.0 |
| ECB6 | 7.6 |
| ECB7 | 7.1 |
| ECB8 | 6.6 |
| ECB9 | 8.1 |
| ECB10 | 6.7 |
| ECB11 | 8.6 |
| ECB15 | 7.6 |
| ECB16 | 7.5 |
| ECB17 | 6.5 |
| ECB18 | 8.0 |
| ECB19 | 6.42 |
| ECB25 | 6.64 |
| ECB29 | 6.92 |
| ECB30 | 6.61 |
| ECB37 | 7.26 |
| ECB44 | 7.48 |
| ECB45 | 7.00 |
| ECB46 | 6.51 |
| ECB47 | 6.24 |
| ECB48 | 7.45 |
| ECB49 | 7.70 |
| ECB50 | 7.62 |
| ECB51 | 7.60 |

Examples EAR2-3, EAR5-12, EAR14-20, EAR22-29, EAR31-46, EAR48-65, EAR67-70, EAR73, EAR75-98, EAR100-104, EAR106-125, EAR127-138, EAR140-160, EAR162, EAR164-175 and EAR177-247, EAR249, EAR2521-273 were tested in the Cereblon Binding Assay. Each of these compounds had a mean pIC50≥6.0.

### Androgen Receptor Degradation Assay Protocol

Degradation of Androgen Receptor protein in human LnCaP cells treated with PROTACs was quantified using the Nano-Glo® HiBiT Lytic Detection System (Promega) in 384 well assay plate format. A clonal LnCaP derived cell line was first established in which the AR gene was modified, using CRISPR/Cas9 editing, so the expressed AR protein included an 11 amino acid HiBiT tag at its amino-terminus. 10 mM DMSO stock solutions of PROTACs were prepared and diluted across an 11 concentration, 3 fold increment range, and 25 nL dispensed into a white opaque bottomed 384 well assay plate (Thermofisher) using an acoustic ECHO dispenser (Labcyte). Cells were growing in cell culture medium (RPMI 1640 supplemented with 15% FBS, penicillin 50 U/mL and streptomycin 50 ug/mL (Thermofisher)). For the assay, cells were detached from flask and resuspended with assay medium (FluoroBrite™ DMEM supplemented with 10% heat inactivated FBS, penicillin 50 U/mL and streptomycin 50 ug/mL (Thermofisher)). The cell suspension was centrifuged at 400 g for 5 min and the pellet resuspended in assay medium. 25 μL of cell suspension containing 10,000 cells was dispensed into each well of the assay plate containing the test compounds, which was then incubated for 18 h at 37° C./5% CO₂. Control wells were included on each assay plate, with assay medium without cells being the no AR remaining 100% effect control, and cells treated with DMSO vehicle only, without PROTAC, the 0% effect control. 25 μL of Nano-Glo® HiBiT lysis buffer supplemented with LgBiT protein and Nano-Glo® substrate (Promega) was added to each well and the plate shaken at 500 rpm for 10 min at room temperature. The intensity of luminescence was measured using a PHERAstar microplate reader (BMG Labtech) and the % AR protein remaining in each well calculated by normalising the raw luminescence value to the above control wells. Parameters corresponding to the potency and efficacy estimates of the PROTACs were obtained from these normalised luminescence values using software (IDBS ActivityBase). These included the DC50 and the Dmax estimates. Two versions of the DC50, called relative DC50 and absolute DC50, were typically generated. These are the inflection point of a fitted 4 parameter sigmoidal response curve and the interpolated concentration of PROTAC corresponding to 50% of the protein being degraded, respectively. The Dmax parameter was the maximum experimentally observed degradation and the asymptote max parameter the fitted response curve asymptote maximum.

The effects of PROTACs on cell viability were estimated by measuring cellular ATP levels using the CellTiter-Glo® Luminescent Cell Viability Assay (Promega). ATP assays were conducted in parallel with AR HiBiT assays on a separate assay plate on the same day, following the same general procedure detailed above.

### Results

Examples EAR1-3, EAR5-20, EAR22-46, EAR48, EAR50-51, EAR53-125, EAR127-138, EAR140-247, EAR249, EAR251-254, EAR257, EAR259-260, EAR268-270, EAR272-273 were tested in the Androgen Receptor Degradation Assay.

Examples EAR1-3, EAR5-20, EAR22-46, EAR48, EAR50-51, EAR53-117, EAR119-125, EAR127-132, EAR134-138, EAR140-247, EAR249, EAR251-254, EAR257, EAR259-260, EAR269-270 and EAR272 exhibited a mean relative pDC50 of ≥6.0.

EAR 118 exhibited a mean pDC50 of 5.72, EAR133 exhibited a mean relative pDC50 of 5.94, EAR268 exhibited a mean relative pDC50 of 5.54 and EAR273 exhibited a mean relative pDC50 of 5.67.

Examples EAR1-3, EAR5-20, EAR22-27, EAR29-46, EAR48, EAR50-51, EAR53-67, EAR70-74, EAR76, EAR78, EAR82-83, EAR86, EAR89, EAR91, EAR93-106, EAR108-117, EAR119-125, EAR127-131, EAR134-137, EAR140-145, EAR147-159, EAR161-163, EAR166-173, EAR175-179, EAR181-221, EAR223-243, EAR246-247, EAR249, EAR251-254, EAR257 and EAR259-260 exhibited a mean relative pDC₅₀ of ≥7.0.

Examples EAR9, EAR11, EAR13, EAR15-16, EAR18, EAR20, EAR22-23, EAR25-27, EAR29-36, EAR38-39, EAR41-44, EAR46, EAR48, EAR50-51, EAR53, EAR56-58, EAR61-62, EAR64-67, EAR70-72, EAR83, EAR97-98, EAR104-106, EAR108-111, EAR114-117, EAR119-120, EAR122-125, EAR127-131, EAR135-136, EAR140-142, EAR144-145, EAR147-149, EAR152, EAR154-157, EAR161-163, EAR166, EAR168, EAR170-173, EAR175, EAR177, EAR179, EAR181-182, EAR185, EAR190-193, EAR196, EAR201, EAR203-205, EAR208-209, EAR211, EAR213-214, EAR216-221, EAR224-227, EAR229-234, EAR236-243, EAR251, EAR253 and EAR259 exhibited a mean relative pDC₅₀ of ≥8.0.

Examples EAR1-3, EAR5-20, EAR22-29, EAR31-46, EAR48, EAR50-51, EAR53-57, EAR59, EAR61-62, EAR64-69, EAR72-78, EAR80-81, EAR83-89, EAR91-104, EAR106-111, EAR114-125, EAR127, EAR130-138, EAR140-159, EAR161-182, EAR184-244, EAR246-247, EAR249, EAR251-253, EAR257, EAR259-260, EAR268-269 and EAR272-273 exhibited a mean asymptote max of ≥50%.

Examples EAR1-3, EAR5-16, EAR18, EAR20, EAR22-26, EAR29, EAR31-35, EAR37, EAR39, EAR43-46, EAR48, EAR50-51, EAR53-57, EAR61-62, EAR65-69, EAR72, EAR75, EAR81, EAR84-87, EAR95, EAR98-102, EAR104, EAR109-111, EAR117-120, EAR122-123, EAR125, EAR127, EAR134-135, EAR140, EAR142, EAR148-149, EAR151-159, EAR161, EAR166-168, EAR170-173, EAR175-176, EAR178-179, EAR181-182, EAR184-191, EAR 193-194, EAR196, EAR198, EAR200-207, EAR209-210, EAR212-217, EAR220-221, EAR225-227, EAR229-231, EAR233-234, EAR237-238, EAR240, EAR242, EAR252-253, EAR268-269 and EAR273 exhibited a mean asymptote max of >70%.

### Dual Mutant (T878A/L702H) Androgen Receptor Degradation Assay Protocol

Degradation of Androgen Receptor dual T878A/L702H mutant protein in human MDA PCa 2b (ATCC, #CRL-2422) cells treated with PROTACs was quantified using the HTRF Human Androgen Receptor Detection Kit (PerkinElmer, #64ANDRPEH) in 384 well assay plate format. 10 mM DMSO(Sigma-Aldrich, #472301) stock solutions of PROTACs were prepared and diluted across an 11 concentration, 3-fold increment range, and 25 nL dispensed into a white opaque bottomed 384 well assay plate (Thermofisher, #164610) using an acoustic ECHO dispenser (Labcyte). Cells were cultured in growth medium (BRFF-HPC-1 (Athena ES, #0403) supplemented with 20% FBS (Gibco, #10099-141), penicillin 50 U/mL and streptomycin 50 ug/mL (Gibco, #15140-122)) in collagen I or poly-D-lysine coated T175 flasks (Corning, #356487 or #356539, respectively). For the assay, cells were harvested from the flask, centrifuged at 200 g for 5 min and then resuspended in the growth medium. 25 μl of cell suspension containing 10,000 cells was dispensed into each well of the assay plate containing the test compounds, which was then incubated for 18 h at 37° C./5% CO₂. Control wells were included on each assay plate, with assay medium without cells being the no AR remaining 100% effect control, and cells treated with DMSO vehicle only, without PROTAC, the 0% effect control. After 18 h incubation 8 μl of supplemented lysis buffer from HTRF kit was added to each well and the plate shaken at 500 rpm for 1 h at room temperature. 8 μl of antibody mix in Detection buffer from HTRF kit was added to each well and the plate was incubated for 2 h in dark at room temperature. The fluorescence was measured at both 620 nm and 665 nm using an excitation wavelength of 337 nm with a PHERAstar microplate reader (BMG Labtech) and the % AR protein remaining in each well was calculated by normalising the HTRF ratio (665 nm signal/620 nm signal) value to the control wells mentioned above. Parameters corresponding to the potency and efficacy estimates of the PROTACs were obtained from these normalised values using curve fitting software (IDBS ActivityBase). These included the DC50 and the Dmax estimates. Two versions of the DC50, called relative DC50 and absolute DC50, were typically generated. These are the inflection point of a fitted 4 parameter sigmoidal response curve and the interpolated concentration of PROTAC corresponding to 50% of the protein being degraded, respectively. The Dmax parameter was the maximum experimentally observed degradation and the asymptote max parameter the fitted response curve asymptote maximum.

The effects of PROTACs on cell viability were estimated by measuring cellular ATP levels using the CellTiter-Glo® Luminescent Cell Viability Assay (Promega, #G7573). ATP assays were conducted in parallel with AR HTRF assays on a separate assay plate on the same day, following manufacturer's instructions and the same general procedure detailed above.

### Results

Examples AR14, AR18, AR29, AR37, AR41, AR67, AR75, AR246-247, AR249, AR251-253 and AR261 were tested in the dual mutant (T878A/L702H) Androgen Receptor Degradation Assay.

Examples AR14, AR18, AR29, AR37, AR41, AR67, AR75, AR246, AR251, AR253 and AR261 exhibited a mean relative pDC50 of ≥6.5. Examples AR18, AR29, AR41, AR67 and AR261 exhibited a mean relative pDC₅₀ of ≥7.0.

Examples AR14, AR18, AR29, AR37, AR41, AR67, AR75, AR246-AR247, AR251-253 and AR261 exhibited a mean asymptote max of ≥55%. Examples AR18, AR29, AR37, AR41, AR75 and AR252-253 exhibited a mean asymptote max of ≥70%.

### RIPK2 Degradation Assay Protocol

Degradation of RIPK2 protein in human embryonic kidney (HEK 293) cells treated with PROTACs was quantified using the Nano-Glo® HiBiT Lytic Detection System (Promega) in 384 well assay plate format. A clonal HEK 293 derived cell line was first established in which the RIPK2 gene was modified, using CRISPR/Cas9 editing, so the expressed RIPK2 protein included an 11 amino acid HiBiT tag at it's amino-terminus. The cell lines were heterozygotic for the HiBIT modification. Cells were cryopreserved in aliquots in 90% foetal bovine serum (FBS), 10% DMSO and stored at −150° C. 10 mM DMSO stock solutions of PROTACs were prepared and diluted across an 11 concentration, 3 fold increment range, and 25 nL dispensed into a white opaque bottomed 384 well assay plate (Thermofisher) using an acoustic ECHO dispenser (Labcyte). For the assay, a cryopreserved vials of cells was rapidly thawed and suspended in 30 mL of assay medium (FluoroBrite™ DMEM supplemented with 10% heat inactivated FBS, penicillin 50 U/mL and streptomycin 50 ug/mL (Thermofisher)). The cell suspension was centrifuged at 400 g for 5 min and the pellet resuspended in assay medium. 25 μL of cell suspension containing 10,000 cells was dispensed into each well of the assay plate containing the test compounds, which was then incubated for 18 h at 37° C./5% CO₂. Control wells were included on each assay plate, with assay medium without cells being the no RIPK2 remaining 100% effect control, and cells treated with DMSO vehicle only, without PROTAC, the 0% effect control. 25 μL of Nano-Glo® HiBiT lysis buffer supplemented with LgBiT protein and Nano-Glo® substrate (Promega) was added to each well and the plate shaken at 500 rpm for 10 min at room temperature. The intensity of luminescence was measured using a PHERAstar microplate reader (BMG Labtech) and the % RIPK2 protein remaining in each well calculated by normalising the raw luminescence value to the above control wells. Parameters corresponding to the potency and efficacy estimates of the PROTACs were obtained from these normalised luminescence values using software (IDBS ActivityBase). These included the DC50 and the Dmax estimates. Two versions of the DC50, called relative DC50 and absolute DC50, were typically generated. These are the inflection point of a fitted 4 parameter sigmoidal response curve and the interpolated concentration of PROTAC corresponding to 50% of the protein being degraded, respectively. The Dmax parameter was the maximum experimentally observed degradation and the asymptote max parameter the fitted response curve asymptote maximum.

### Results

Examples R1-3 were tested in the RIPK2 Degradation Assay. Data for individual compounds is set out below:

| | Mean | Mean |
|---|---|---|
| | asymptote | relative |
| Example | max (%) | pDC₅₀ |
| R1 | 96 | 8.5 |
| R2 | 88 | 8.5 |
| R3 | 92 | 8.3 |

### IRAK4 Degradation Assay Protocol

Degradation of IRAK4 (Interleukin 1 Receptor Associated Kinase 4) protein in Hela cells treated with PROTACs was quantified using the Nano-Glo® HiBiT Lytic Detection System (Promega) in 384 well assay plate format. A clonal HeLa cell line was first established in which the IRAK4 gene was modified, using CRISPR/Cas9 editing, so the expressed IRAK4 protein included an 11 amino acid HiBiT tag at it's amino-terminus. The cell lines were heterozygotic for the HiBiT modification. Cells were cryopreserved in aliquots in 90% foetal bovine serum (FBS), 10% DMSO and stored at −150° C. 10 mM DMSO stock solutions of PROTACs were prepared and diluted across an 11 concentration, 3 fold increment range, and 25 nL dispensed into a white opaque bottomed 384 well assay plate (Thermofisher) using an acoustic ECHO dispenser (Labcyte). For the assay, a cryopreserved vials of cells was rapidly thawed and suspended in 30 ml of assay medium (FluoroBrite™ DMEM supplemented with 10% heat inactivated FBS, penicillin 50 U/mL and streptomycin 50 ug/mL (Thermofisher)). The cell suspension was centrifuged at 400 g for 5 min and the pellet resuspended in assay medium. 25 μL of cell suspension containing 10,000 cells was dispensed into each well of the assay plate containing the test compounds, which was then incubated for 18 h at 37° C./5% CO₂. Control wells were included on each assay plate, with assay medium without cells being the no IRAK4 remaining 100% effect control, and cells treated with DMSO vehicle only, without PROTAC, the 0% effect control. 25 uL of Nano-Glo® HiBiT lysis buffer supplemented with LgBiT protein and Nano-Glo® substrate (Promega) was added to each well and the plate shaken at 500 rpm for 10 min at room temperature. The intensity of luminescence was measured using a PHERAstar microplate reader (BMG Labtech) and the % IRAK4 protein remaining in each well calculated by normalising the raw luminescence value to the above control wells. Parameters corresponding to the potency and efficacy estimates of the PROTACs were obtained from these normalised luminescence values using software (IDBS ActivityBase). These included the DC50 and the Dmax estimates. Two versions of the DC50, called relative DC50 and absolute DC50, were typically generated. These are the inflection point of a fitted 4 parameter sigmoidal response curve and the interpolated concentration of PROTAC corresponding to 50% of the protein being degraded, respectively. The Dmax parameter was the maximum experimentally observed degradation and the asymptote max parameter the fitted response curve asymptote maximum.

### Results

Example I1 was tested in the IRAK4 Degradation Assay. It exhibited a mean relative pDC50 of 8.1 and a mean asymptote max of 96%.

What is claimed is:

1-46. (canceled)

47. A compound of formula (I), or a tautomer of a compound of formula (I), or a salt thereof: 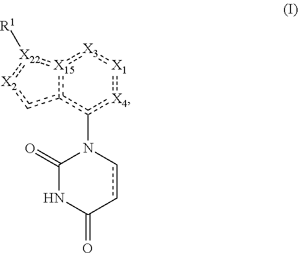
wherein:
X₁ is N or C—R² wherein R² is selected from the group consisting of hydrogen, halogen, C₁₋₄alkyl, C₁₋₄haloalkyl, C₁₋₄alkoxy, —CONR⁵R⁶ and —(CONR³R⁴)m(L)p(TBM)q;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
n is 0 or 1;
R⁵ and R⁶ are independently selected from hydrogen or C₁₋₄alkyl;
R¹ is C₁₋₄alkyl, C₁₋₄haloalkyl, C₁₋₄alkoxy or a group of formula (II), wherein * represents the position of attachment to the compound of formula (I): 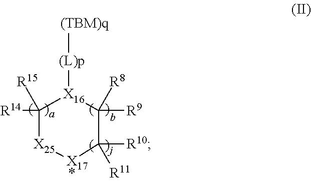
a and b are independently 0 or 1;
X₁₆ is N or CH;
X₁₇ is CR³⁴R³⁵ wherein either R³⁴ and R³⁵ together with the carbon atom to which they are attached, join together to form a cyclobutyl ring, or wherein R³⁴ is —(CHR³⁶)r-, or a bond to the compound of formula (I) and R³⁵ is hydrogen or halogen;
X₂₅ is CR¹²R¹³ or O;
r is 0, 1 or 2;
a is 0, 1 or 2 and b and j are independently 0 or 1 with the proviso that b and j cannot both be 0;
R³⁶ is hydrogen or methyl;
R⁸, R⁹, R¹⁰, R¹¹, R¹², R¹³, R¹⁴ and R¹⁵ are independently selected from hydrogen or halogen;
L is a chemical linker;
TBM is a target binding moiety;
m, p and q are independently 0 and 1;
wherein when X₁₅ is N, then X₂₂ is C; and
wherein when X₁ is —(CONR³R⁴)m(L)p(TBM)q, then R¹ is C₁₋₄alkyl, C₁₋₄haloalkyl or C₁₋₄alkoxy.

48. A compound of formula (I), or a tautomer or a salt thereof, according to claim 47, which has formula (Ia): 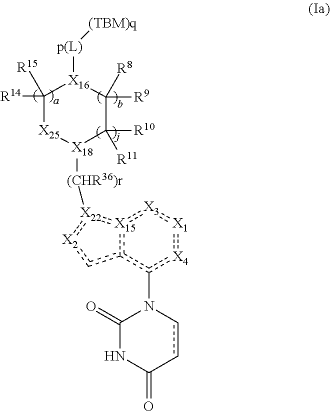
wherein:
X₁ is N or C—R² wherein R² is selected from the group consisting of hydrogen, halogen, C₁₋₄alkyl, C₁₋₄haloalkyl, C₁₋₄alkoxy and —CONR⁵R⁶;
X₄ is C—R⁷ or N wherein R⁷ is selected from the group consisting of hydrogen, halogen, C₁₋₃alkyl, C₁₋₃haloalkyl and C₁₋₃alkoxy;
X₂ and X₃ are independently selected from N or CH;
X₁₅ and X₂₂ are independently selected from N or C;
r is 0, 1 or 2;
a is 0, 1 or 2 and b and j are independently 0 or 1 with the proviso that b and j cannot both be 0;
X₁₆ is N or CH;
X₁₈ is CR³⁵, wherein R³⁵ is hydrogen or halogen;
X₂₅ is CR¹²R¹³ or O;
R⁵ and R⁶ are independently selected from hydrogen or C₁₋₄alkyl;
R⁸, R⁹, R¹⁰, R¹¹, R¹², R¹³, R¹⁴, R¹⁵ and R³⁵ are independently selected from hydrogen or halogen;
R³⁶ is hydrogen or methyl;
L is a chemical linker;
TBM is a target binding moiety;
p and q are independently 0 and 1; and
wherein when X₁₅ is N, then X₂₂ is CH.

49. A compound of formula (Ia), or a tautomer or a salt, thereof according to claim 48, wherein X₁ is N or CH.

50. A compound of formula (Ia), a tautomer or a salt thereof, according to claim 48, wherein X₄ is C—R7.

51. A compound of formula (Ia), or a tautomer or a salt thereof, according to claim 48, wherein X₄ is N.

52. A compound of formula (Ia), or a tautomer or a salt thereof, according to claim 48, wherein X₂ is CH.

53. A compound of formula (Ia), or a tautomer or a salt thereof, according to claim 48, wherein X₃ is CH.

54. A compound of formula (Ia), or a tautomer or a salt thereof, according to claim 48, wherein X₁₅ is C.

55. A compound of formula (Ia), or a tautomer or a salt thereof, according to claim 48, wherein X₂₂ is N.

56. A compound of formula (Ia), or a tautomer or a salt thereof, according to claim 48, wherein X₁₆ is N, X₂₅ is CR¹²R¹³, j is 1 and a and b are independently 0 or 1.

57. A compound of formula (Ia), or a tautomer or a salt thereof, according to claim 48, wherein r is 0.

58. A compound of formula (Ia), or a tautomer or a salt thereof, according to claim 48, wherein:
R⁸, R⁹ and R³⁵ are independently selected from hydrogen or halogen; and
R¹⁰, R¹¹, R¹², R¹³, R¹⁴ and R¹⁵ are each hydrogen.

59. A compound of formula (Ia), or a tautomer or a salt thereof, according to claim 48, wherein q is 1.

60. A pharmaceutical composition comprising the compound, or a tautomer or a pharmaceutically acceptable salt thereof, according to claim 59 and a pharmaceutically acceptable excipient.

61. A compound of formula (Ia), or a tautomer or a salt thereof, according to claim 48, which has the formula (Iaaaa):

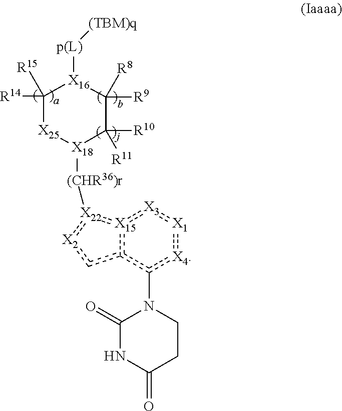

62. A compound of formula (Iaaaa), or a tautomer or a salt thereof, according to claim 61, wherein q is 1.

63. A pharmaceutical composition comprising the compound, tautomer or a pharmaceutically acceptable salt thereof, according to claim 62 and a pharmaceutically acceptable excipient.

64. A compound of formula (I), or a tautomer or a salt thereof, according to claim 47, which has formula (Ib) 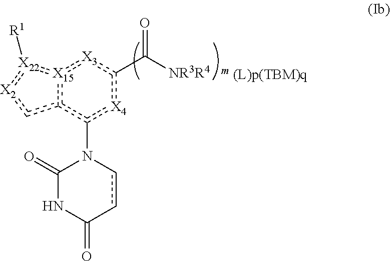
wherein:
R¹ is C₁₋₄alkyl, C₁₋₄haloalkyl or C₁₋₄alkoxy, wherein said C₁₋₄alkyl group is optionally substituted by one C₁₋₄alkoxy group;
X₂ and X₃ are independently selected from N or CH;
X₁₅ and X₂₂ are independently selected from N or C;
X₄ is C—R⁷ or N wherein R⁷ is selected from the group consisting of hydrogen, halogen, C₁₋₃alkyl, C₁₋₃haloalkyl and C₁₋₃alkoxy;
either R³ and R⁴ together with the nitrogen atom to which they are attached, join together to form a monocyclic or spiro nitrogen containing heterocyclic ring, or R³ is H or C₁₋₄alkyl and R⁴ is —(CH₂)ₙR²⁸, wherein R²⁸ is a monocyclic or spiro nitrogen containing heterocyclic ring;
m is 0 or 1;
n is 0 or 1;
L is a chemical linker;
TBM is a target binding moiety; and
p and q are independently 0 and 1; and
wherein when X₁₅ is N, then X₂₂ is CH.

65. A compound of formula (Ib), or a tautomer or a salt thereof, according to claim 64, wherein X₄ is N or CH.

66. A compound of formula (Ib), or a tautomer or a salt thereof, according to claim 65, wherein X₄ is CH.

67. A compound of formula (Ib), or a tautomer or a salt thereof, according to claim 64, wherein X₂ is CH.

68. A compound of formula (Ib), or a tautomer or a salt thereof, according to claim 64, wherein X₃ is CH.

69. A compound of formula (Ib), or a tautomer or a salt thereof, according to claim 64, wherein X₁₅ is C.

70. A compound of formula (Ib), or a tautomer or a salt thereof, according to claim 64, wherein X₂₂ is N.

71. A compound of formula (Ib), or a tautomer or a salt thereof, according to claim 64, wherein m is 1.

72. A compound of formula (Ib), or a tautomer or a salt thereof, according to claim 64, wherein R³ is H or C₁-4alkyl and R⁴ is —(CH2)nR28, wherein R²⁸ is a monocyclic nitrogen containing heterocyclic ring.

73. A compound of formula (Ib), or a tautomer or a salt thereof, according to claim 64, wherein the group NR³R⁴, has a structure selected from the following: 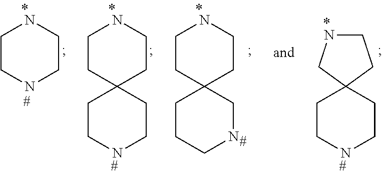
wherein the asterisk represents the position of attachment to the carbonyl group and the # represents the point of attachment to L.

74. A compound of formula (Ib), or a tautomer or salt thereof, according to claim 64, wherein R1 is C1-4alkyl.

75. A compound of formula (Ib), or a tautomer or salt thereof, according to claim 74, wherein R1 is isopropyl.

76. A compound of formula (Ib), or a tautomer or salt thereof, according to claim 64, wherein q is 1.

77. A pharmaceutical composition comprising the compound, a tautomer or a pharmaceutically acceptable salt thereof, according to claim 76 and a pharmaceutically acceptable excipient.

78. A compound of formula (Ib), or a tautomer or salt thereof, according to claim 64, which has the formula (Ibbbb):

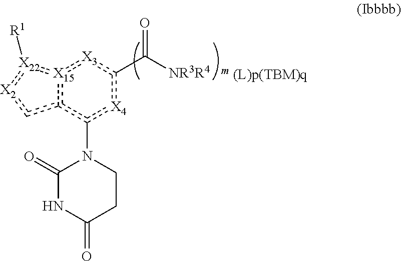

79. A compound of formula (Ibbbb), or a tautomer or salt thereof, according to claim 68, wherein q is 1.

80. A pharmaceutical composition comprising the compound, a tautomer or pharmaceutically acceptable salt thereof, according to claim 79 and a pharmaceutically acceptable excipient.

81. A compound of formula (Iaaaa), or a tautomer or salt thereof, according to claim 62, wherein the TBM is an androgen receptor binding moiety.

82. A pharmaceutical composition comprising the compound, tautomer or a pharmaceutically acceptable salt thereof, according to claim 81 and a pharmaceutically acceptable excipient.

83. A method of treating a disorder selected from the group consisting of cancer, benign prostatic hyperplasia, ovarian cysts, polycystic ovary syndrome and Kennedy's Disease, comprising administering to a human in need thereof, a therapeutically effective amount of the compound, tautomer or a pharmaceutically acceptable salt thereof, as defined in claim 81 or the pharmaceutical composition according to claim 82.
